(19) United States
(12) Patent Application Publication
(10) Pub. No.: US 2024/0043395 A1
(43) Pub. Date: Feb. 8, 2024

(54) COMPOUNDS AND COMPOSITIONS AS MODULATORS OF TLR SIGNALING

(71) Applicant: Neuropore Therapies, Inc., San Diego, CA (US)

(72) Inventors: Srinivasa Reddy NATALA, San Diego, CA (US); Wolfgang J. WRASIDLO, La Jolla, CA (US); Emily M. STOCKING, Encinitas, CA (US)

(21) Appl. No.: 18/027,005

(22) PCT Filed: Sep. 24, 2021
(86) PCT No.: PCT/US2021/052074

§ 371 Date: Mar. 17, 2023

Related U.S. Application Data
(60) Provisional application No. 63/083,685, filed on Sep. 25, 2020.

(51) Int. Cl. C07D 285/135; C07D 277/46; C07D 277/56; C07D 295/112; C07D 263/48; C07D 277/24; C07D 213/75; C07D 211/26; C07D 295/16; C07D 207/09; C07D 205/04; C07D 237/20; C07D 231/12; C07D 417/14; C07D 417/04; C07F 9/6558; C07D 213/74; C07D 295/135; C07D 265/30; C07D 279/12; C07C 235/62; C07C 243/38; C07D 211/96; C07D 417/12; C07D 417/10; C07D 285/08; C07D 241/04; C07D 221/20; C07D 401/04

(52) U.S. Cl. CPC C07D 285/135; C07D 401/04; C07D 277/56; C07D 295/112; C07D 263/48; C07D 277/24; C07D 213/75; C07D 211/26; C07D 295/16; C07D 207/09; C07D 205/04; C07D 237/20; C07D 231/12; C07D 417/14; C07D 417/04; C07F 9/65583; C07D 213/74; C07D 295/135; C07D 265/30; C07D 279/12; C07C 235/62; C07C 243/38; C07D 211/96; C07D 417/12; C07D 417/10; C07D 285/08; C07D 241/04; C07D 221/20; C07D 277/46

(57) ABSTRACT

The present disclosure relates to compounds, pharmaceutical compositions comprising such compounds, and use of such compounds in methods of treatment or in medicaments for treatment of inflammatory diseases and certain neurological disorders that are related to inflammatory signaling processes, including but not limited to misfolded proteins.

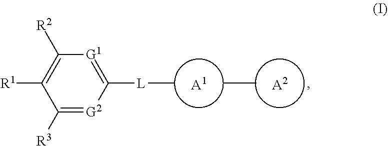

### CROSS-REFERENCE TO RELATED APPLICATIONS

This application claims priority to U.S. Provisional Application No. 63/083,685, filed Sep. 25, 2020, entitled “COMPOUNDS AND COMPOSITIONS AS MODULATORS OF TLR SIGNALING” the contents of which are hereby incorporated by reference in their entirety for all purposes.

### TECHNICAL FIELD

The present disclosure relates to compounds, pharmaceutical compositions comprising such compounds, and use of such compounds in methods of treatment or in medicaments for treatment of inflammatory diseases and certain neurological disorders that are related to inflammatory signaling processes, including but not limited to misfolded proteins.

### BACKGROUND

Toll-like receptors (TLRs) are sentinel receptors of the immune system. When these receptors are activated on cell surfaces, they initiate recruitment of a family of TIR-domain containing adapter proteins, which induce a signaling cascade that ultimately results in cell-type specific inflammatory responses, resulting in the elevation of pro-inflammatory mediators such as IL1, IL6, IL8 and TNFα. Of the different TLR receptors expressed on mammalian cells, TLR2 forms heterodimers with either TLR1 or TLR6 to initiate inflammatory responses with various microbial derived ligands. Among the various bacterial ligands are lipopolysaccharides (LPS), acylated lipopeptides, lipoglycans, peptidoglycans, porins, glycosylphosphatidyl-inosol anchors, and other bacterial cell wall components such as lipoteichoic acid (LTA) from streptococous pneumonia. In addition to the microbial activation of TLR2, it has also been found that abnormal aggregation of neuron released oligomeric proteins such as alpha-synuclein (aSyn) can induce similar inflammatory responses in animal models of neurodegenerative diseases, including Parkinson's disease (PD), dementia with Lewy bodies, multiple system atrophy (MSA) and Alzheimer's disease (AD). See, e.g., Kim et al., Nat. Commun. 2013, 4, 1562.

The ability of TLR2 to induce signaling via heterodimers allows discrimination between various recognition patterns, which allows for the design of ligands with specific inhibition patterns. Kajava et al., J. Biol. Chem. 2010, 285, 6227. Inhibitors that compete primarily with a specific pathological agonist, such as oligomeric pathogenic alpha-synuclein, but do not affect other ligands involved in pro-inflammatory signaling of bacterial or viral infections or non-competitive TIR-Myd88 inhibitors, such as compounds that function indirectly as non-competitive inhibitors of TLR2 though intracellualar TIR-Myd88 inhibition, would therefore be useful as potential therapeutic agents.

The function of Toll-like receptors has been linked to various protein folding, protein dimerization, and inflammatory processes and to related diseases such as Alzheimer's disease (Gambuzza, M. et al., “Toll-like receptors in Alzheimer's disease: a therapeutic perspective,” CNS Neurol. Disord. Durg Targets 2014, 13(9), 1542-58), Parkinson's disease and Parkinson's disease with dementia (Beraud, D. et al., “Misfolded α-synuclein and Toll-like receptors: therapuetic targets for Parkinson's disease,” Parkinsonism Relat. Disord. 2012, 18 (Suppl. 1), S17-20), fronto-temporal dementia, dementia with Lewy bodies (Lewy body disease), multiple system atrophy (Vieira, B. et al., “Neuroinflammation in multiple system atrophy: Response to and cause of α-synuclein aggregation,” Front. Cell Neurosci. 2015, 9, 437), amyotrophic lateral sclerosis (Casula, M. et al., “Toll-like receptor signaling in amyotrophic lateral sclerosis spinal cord tissue,” Neuroscience 2011, 179, 233-43), Huntington's disease (Kalathur, R. K. R. et al., “Huntington's disease and its therapeutic target genes: a global functional profile based on the HD Research Crossroads database,” BMC Neurology 2012, 12, 47), inflammatory diseases, asthma and chronic obstructive pulmonary disease (COPD) (Zuo, L. et al., “Molecular regulation of Toll-like receptors in asthma and COPD,” Front. Physiol. 2016, 6, 312), chronic peptic ulcers (Smith, S., “Roll of Toll-like receptors in Helicobacter pylori infection and immunity,” World J. Gastrointest. Pathophysiol. 2014, 5(3), 133-146), tuberculosis (Harding, C. V. et al., “Regulation of antigen presentation by Mycobacterium tuberculosis: a role for Toll-like receptors,” Nat. Rev. Microbiol. 2010, 8(4), 296-307), rheumatoid arthritis (Huang, Q.-Q. et al., “Roll of Toll like receptors in rheumatoid arthritis,” Curr. Rheumatol. Rep. 2009, 11(5), 357-364), chronic sinusitis (Zhang, Q. et al., “Differential expression of Toll-like receptor pathway genes in chronic rhinosinusitis with or without nasal polyps,” Acta Otolaryngol. 2013, 133(2), 165-173), hepatitis (including hepatitis B and C) (Zhang, E. et al., “Toll-like receptor (TLR)-mediated innate immune responses in control of hepatitis B virus (HBV) infection,” Med. Microbiol. Immunol. 2015, 204(1), 11-20; Howell, J. et al., “Toll-like receptors in hepatitis C infection: implications for pathogenesis and treatment,” J. Gastroenterol. Hepatol. 2013, 28(5), 766-776), gout, lupus, psoriasis, psoriatic arthritis (Santegoets, K. C. M. et al., “Toll-like receptors in rheumatic diseases: are we paying a high price for our defense against bugs?” FEBS Letters 2011, 585(23), 3660-3666), vasculitis, laryngitis, pleurisy (Chen, X. et al., “Engagement of Toll-like receptor 2 on CD4(+) T cells facilitates local immune responses in patients with tuberculous pleurisy,” J. Infect. Dis. 2009, 200(3), 399-408), eczema (Miller, L. S., “Toll-like receptors in skin,” Adv. Dermatol. 2008, 24, 71-87), gastritis (Schmausser, B. et al., “Toll-like receptors TLR4, TLR5 and TLR9 on gastric carcinoma cells: an implication for interaction with Helicobacter pylori,” Int. J. Med. Microbiol. 2005, 295(3), 179-85), vasculitis (Song, G. G. et al., “Toll-like receptor polymorphisms and vasculitis susceptibility: meta-analysis and systematic review,” Mol. Biol. Rep. 2013, 40(2), 1315-23), laryngitis (King, S. N. et al., “Characterization of the Leukocyte Response in Acute Vocal Fold Injury,” PLoS One, 2015; 10(10): e0139260), allergic reactions (Gangloff, S. C. et al., “Toll-like receptors and immune response in allergic disease,” Clin. Rev. Allergy Immunol. 2004, 26(2), 115-25), multiple sclerosis (Miranda-Hernandez, S. et al., “Role of toll-like receptors in multiple sclerosis,” Am. J. Clin. Exp. Immunol. 2013, 2(1), 75-93), Crohn's disease (Cario, E., “Toll-like receptors in inflammatory bowel diseases: A decade later,” Inflamm. Bowel Dis. 2010, 16(9), 1583-1597), and traumatic brain injury (Hua, F. et al., “Genomic profile of Toll-like receptor pathways in traumatically brain-injured mice: effect of exogenous progesterone,” J. Neuroinflammation 2011, 8, 42).

The signal transduction path of TLR2 can be activated either through the external domain (agonist pocket) or by mechanisms involving the cytoplasmic TIR domain that mediates homotypic and heterotypic interactions during signaling. The proteins MyD88 and TIRAP (Mal) are involved in this type of signaling.

Importantly, a conserved proline P681 in TLR2 within the BB loop is (Brown V. et. al. (2006) European Journal of immunology 36, 742-753) is involved in the dimerization mechanism. A mutation in this loop from P681H abolishes recruitment of MyD88 and signaling. Thus compounds that bind in the vicinity of this loop and restrict its movement during the dimerization process would be useful as inhibitors of the activation of TLR2.

TLR9 is a pattern recognition receptor involved in host defense mechanisms. The persistent or inappropriate activation of TLR9 has been implicated in a number of different central nervous system (CNS) and peripheral disorders. Thus inhibition of TLR9, either alone or in combination with TLR2 blockade may provide therapeutic benefit. CNS disorders where TLR9 has been implicated include Parkinson's disease (Maatouk et al., Nat Commun. 2018, Jun. 22; 9(1):2450); Amyotrophic lateral sclerosis (O'Rourke et al., Science. 2016, Mar. 18; 351(6279):1324-9); Guillain-Barre syndrome (Wang et al., Immunol Invest. 2011, 2012; 41(2):171-82); spinal cord injury (Li et al., Brain Behav Immun. 2019 August; 80:328-343; Li et al., J. Neuroinflammation. 2020 Feb. 25; 17(1):73; David et al., Neurobiol Dis. 2013 June; 54:194-205; Pallottie et al., Sci Rep. 2018 Jun. 7; 8(1):8723) and; multiple sclerosis (Prinz et al., J Clin Invest. 2006 February; 116(2):456-64). Peripheral disorders where TLR9 has been implicated are wide ranging and include multiple forms of tissue injury (mcAlpine et al., Proc Natl Acad Sci USA. 2018 Dec. 4; 115(49):E11523-E11531), chronic pain (David et al., Neurobiol Dis. 2013 June; 54:194-205), and psoriasis (Balak et al., Clin Immunol. 2017 January; 174:63-72).

Described herein are compounds that serve as antagonists of TLR2 and/or inhibitors of TLR9 with high potency and selectivity.

### SUMMARY

In one aspect, provided are compounds of Formula (I):

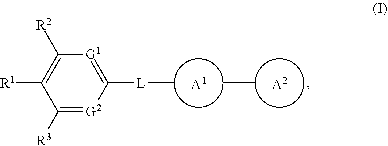

or a tautomer thereof, or a pharmaceutically acceptable salt of any of the foregoing,
wherein
R¹ is R^{1A} and R² is R^{2A}, or R¹ is R^{2A} and R² is R^{1A},
   wherein R^{1A} is —OH, —OPO₃H₂, —OCH₂OPO₃H₂, —OC(O)R^{1A1}, —OC(O)OR^{1A1}, —OC(O)NHR^{1A1}, —OC(O)NR^{1A1}R^{1A2}, or —OR^{1A3},
      wherein R^{1A1} and R^{1A2} are each independently hydrogen, optionally substituted alkyl, optionally substituted alkenyl, optionally substituted alkynyl, optionally substituted cycloalkyl, optionally substituted cycloalkenyl, optionally substituted aryl, optionally substituted heterocyclyl, optionally substituted heteroaryl or —O₀₋₁(CH₂)ₘO(CH₂)ₙOH, wherein m and n are each independently 1 or 2, and;
      R^{1A3} is optionally substituted heteroaryl;
   R^{2A} is —CHO or —CH═NR^{2A1},
      wherein R^{2A1} is optionally substituted heterocyclyl, optionally substituted alkyl, optionally substituted alkenyl, optionally substituted alkynyl, —NR^{2A1A}C(O)R^{2A1B}, —NR^{2A1A}S(O)₂R^{2A1B}, —NR^{2A1A}R^{2A1B}, —OR^{2A1A} or —NR^{2A1A}C(NR^{2A1B})NR^{2A1C}R^{2A1D}, and
      wherein R^{2A1A}, R^{2A1B}, R^{2A1C}, and R^{2A1D} are each independently hydrogen, optionally substituted alkyl, optionally substituted cycloalkyl, optionally substituted heteroaryl, optionally substituted aryl, or optionally substituted amino; or
   R^{1A} and R^{2A} taken together with

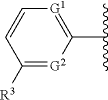

to which they are attached form optionally substituted

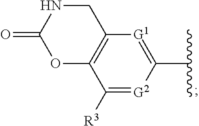

R³ is halo, hydrogen, optionally substituted alkyl, or optionally substituted alkoxy;
G¹ and G² are each independently CH or N;
L is a bond, —C(O)NH—*, —NHC(O)—*, —C(R^{4A})(R^{4B})NHC(O)—*, —C(O)—, —S(O)₂—, —S(O)₂NH—*,

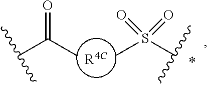

—C(O)N(R^{4D})(CH₂)₂₋₃—*, —C(O)N(CH₃)—*, —(CH₂)OC(O)NH—*, —C(O)NHNH—*, —C(O)NHNHC(O)—*, —CH(R^{4E})NHC(O)O—*, or —C(O)NHO—*,
   wherein R^{4A}, R^{4B}, R^{4D}, and R^{4E} are each independently hydrogen or optionally substituted alkyl,
   R^{4C} is optionally substituted aryl, optionally substituted cycloalkyl, optionally substituted cycloalkenyl, optionally substituted heteroaryl, or optionally substituted heterocyclyl, and
   represents the point of attachment to A¹; and
A¹ and A² are each independently optionally substituted aryl, optionally substituted cycloalkyl, optionally substituted cycloalkenyl, optionally substituted heteroaryl, or optionally substituted heterocyclyl.

In some embodiments, when R¹ is —OH, R³ is fluoro, L is a bond, and A¹ is optionally substituted 5-membered heteroaryl, then A² is not optionally substituted phenyl, optionally substituted pyridinyl, optionally substituted pyrazinyl, or 2,3-dihydrobenzo[b][1,4]dioxin-6-yl; when R¹ is —CHO, R² is —OH, R³ is hydrogen, and L is —C(O), then A¹ is not optionally substituted indolinyl; when L is —C(O)NH—*, then A¹ is not optionally substituted phenyl, optionally substituted pyridinyl, or pyrimidinyl; when R³ is hydrogen, C₁₋₄ alkyl, —CHO, or methoxy, then L is not a bond; and the compound of Formula (I) is not 3-fluoro-5-formyl-4-hydroxy-N-(4-(pyrrolidin-1-yl)phenyl)benzenesulfonamide, 5-(4-(5-fluoropyridin-2-yl)piperazine-1-carbonyl)-2-hydroxy-3-methylbenzaldehyde, 5-(3-(1λ⁴,2λ²,4-triazol-1-yl)azetidine-1-carbonyl)-2-hydroxy-3-methylbenzaldehyde, tert-butyl (3-(1-(3-formyl-4-hydroxybenzoyl)piperidin-4-yl)benzyl)carbamate, 5-(4-cyclopropyl-3-oxopiperazine-1-carbonyl)-2-hydroxy-3-methylbenzaldehyde, 2-(5-((((4-formyl-3-hydroxybenzyl)oxy)carbonyl)amino)benzol[d]thiazol-2-yl)-4,5-dihydrothiazole-4-carboxylic acid, 4-formyl-3-hydroxybenzyl (6-(benzo[d]oxazol-2-yl)naphthalen-2-yl)carbamate, 5-[2-(3,4-diethoxyphenyl)-4-thiazolyl]-3-formyl-2-hydroxy-benzoic acid methyl ester, or a salt of any of the foregoing.

In a further aspect, provided herein are pharmaceutical compositions comprising at least one compound of Formula (I), such as a compound of Table 1, or a tautomer thereof, or a pharmaceutically acceptable salt of any of the foregoing, optionally further comprising a pharmaceutically acceptable excipient.

In another aspect, provided herein is a method of treating a disease or condition associated with TLR2, comprising administering to a subject in need of such treatment an effective amount of at least one compound of Formula (I), such as a compound of Table 1, or a tautomer thereof, or a pharmaceutically acceptable salt of any of the foregoing, and/or a pharmaceutical composition comprising at least one compound of Formula (I), such as a compound of Table 1. In some embodiments of any of the methods described herein, the disease or condition is selected from the group consisting of: Alzheimer's disease, Parkinson's disease, fronto-temporal dementia, dementia with Lewy bodies (Lewy body disease), Parkinson's disease with dementia, multiple system atrophy, amyotrophic lateral sclerosis, Huntington's disease, Progressive Supranuclear Palsy (PSP), Niemann-Pick disease type C, Guillain-Barré syndrome (GBS), Barrett's esophagus, inflammatory diseases, asthma, chronic obstructive pulmonary disease (COPD), chronic peptic ulcers, irritable bowel disease, tuberculosis, rheumatoid arthritis, osteoarthritis, chronic sinusitis, hepatitis, hepatitis B, hepatitis C, gout, lupus, pleurisy, eczema, gastritis, psoriasis, psoriatic arthritis, vasculitis, laryngitis, allergic reactions, multiple sclerosis, Crohn's disease, traumatic brain injury, CIDP (chronic inflammatory demyelinating polyneuropathy), stroke, ischemic heart disease, atopic dermatitis, acne vulgaris, rosacea, non-alcoholic fatty liver disease, non-alcoholic steatohepatisis, corneal wounds, corneal disorders, corneal HSV, Stargardt disease (Juvenile macular degeneration), age-related macular degeneration, sepsis, diabetic wounds, herpes simplex virus, and anti-fungal, anti-bacterial, anitviral and antitumor diseases or conditions.

In yet another aspect, provided herein is a method of interfering with the heterodimerization of TLR2 in a cell, or modulating, preventing, slowing, reversing, or inhibiting TLR2 heterodimerization in a cell, comprising contacting the cell with an effective amount of at least one compound of Formula (I), such as a compound of Table 1, or a tautomer thereof, or a pharmaceutically acceptable salt of any of the foregoing, and/or with at least one pharmaceutical composition comprising at least one compound of Formula (I), such as a compound of Table 1, wherein the contacting is in vitro, ex vivo, or in vivo.

In another aspect, provided herein is a method of treating a disease or condition associated with inhibition of TLR9, comprising administering to a subject in need of such treatment an effective amount of at least one compound of Formula (I), such as a compound of Table 1, or a tautomer thereof, or a pharmaceutically acceptable salt of any of the foregoing, and/or a pharmaceutical composition comprising at least one compound of Formula (I), such as a compound of Table 1. In some embodiments of any of the methods described herein, the disease or condition is a central nervous sytem (CNS) or peripheral disorder. In some embodiments, the CNS disorder is Parkinson's disease, Amyotrophic lateral sclerosis, Guillain-Barre syndrome, spinal cord injury, or multiple sclerosis. In some embodiments, the peripheral disorders include multiple forms of tissue injury, chronic pain, and psoriasis.

Additional embodiments, features, and advantages of the present disclosure will be apparent from the following detailed description and through practice of the present disclosure.

For the sake of brevity, the disclosures of publications cited in this specification, including patents, are herein incorporated by reference.

### DETAILED DESCRIPTION

The present disclosure relates to compounds, pharmaceutical compositions comprising such compounds, and use of such compounds in methods of treatment or in medicaments for treatment of inflammatory diseases and certain neurological disorders that are related to inflammatory signaling processes, including but not limited to misfolded proteins.

It is to be understood that this disclosure is not limited to particular embodiments described, as such may, of course, vary. It is also to be understood that the terminology used herein is for the purpose of describing particular embodiments only, and is not intended to be limiting.

Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

Throughout this application, unless the context indicates otherwise, references to a compound of Formula (I) includes all subgroups of Formula (I) defined herein, such as Formula (I-1), (Ia-1), (Ia-2), (Ia-3), (Ib-1), (Ib-2), (Ib-3), (Ic-1), (Ic-2), (Id-1), or (Id-2), including all substructures, subgenera, preferences, embodiments, examples and particular compounds defined and/or described herein. In some embodiments, references to a compound of Formula (I) and subgroups thereof, such as Formula (I-1), (Ia-1), (Ia-2), (Ia-3), (Ib-1), (Ib-2), (Ib-3), (Ic-1), (Ic-2), (Id-1), or (Id-2), include ionic forms, polymorphs, pseudopolymorphs, amorphous forms, solvates, co-crystals, chelates, isomers, tautomers, oxides (e.g., N-oxides, S-oxides), esters, prodrugs, isotopes and/or protected forms thereof. In some embodiments, references to a compound of Formula (I) and subgroups thereof, such as Formula (I-1), (Ia-1), (Ia-2), (Ia-3), (Ib-1), (Ib-2), (Ib-3), (Ic-1), (Ic-2), (Id-1), or (Id-2), include polymorphs, solvates, co-crystals, isomers, tautomers and/or oxides thereof. In some embodiments, references to a compound of Formula (I) and subgroups thereof, such as Formula (I-1), (Ia-1), (Ia-2), (Ia-3), (Ib-1), (Ib-2), (Ib-3), (Ic-1), (Ic-2), (Id-1), or (Id-2), include polymorphs, solvates, and/or co-crystals thereof. In some embodiments, references to a compound of Formula (I) and subgroups thereof, such as Formula (I-1), (Ia-1), (Ia-2), (Ia-3), (Ib-1), (Ib-2), (Ib-3), (Ic-1), (Ic-2), (Id-1), or (Id-2), include isomers, tautomers and/or oxides thereof. In some embodiments, references to a compound of Formula (I) and subgroups thereof, such as Formula (I-1), (Ia-1), (Ia-2), (Ia-3), (Ib-1), (Ib-2), (Ib-3), (Ic-1), (Ic-2), (Id-1), or (Id-2), include solvates thereof.

As used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As used herein, the terms “including,” “containing,” and “comprising” are used in their open, non-limiting sense.

To provide a more concise description, some of the quantitative expressions given herein are not qualified with the term “about”. It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. Whenever a yield is given as a percentage, such yield refers to a mass of the entity for which the yield is given with respect to the maximum amount of the same entity that could be obtained under the particular stoichiometric conditions. Concentrations that are given as percentages refer to mass ratios, unless indicated differently.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure. All publications mentioned herein are incorporated herein by reference to disclose and describe the methods and/or materials in connection with which the publications are cited.

Except as otherwise noted, the methods and techniques of the present embodiments are generally performed according to conventional methods well known in the art and as described in various general and more specific references that are cited and discussed throughout the present specification. See, e.g., Loudon, Organic Chemistry, Fourth Edition, New York: Oxford University Press, 2002, pp. 360-361, 1084-1085; Smith and March, March's Advanced Organic Chemistry: Reactions, Mechanisms, and Structure, Fifth Edition, Wiley-Interscience, 2001.

The nomenclature used herein to name the subject compounds is illustrated in the Examples herein. This nomenclature has generally been derived using the commercially-available ChemBioDraw Ultra software, Version 14.0.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination. All combinations of the embodiments pertaining to the chemical groups represented by the variables are specifically embraced by the present disclosure and are disclosed herein just as if each and every combination was individually and explicitly disclosed, to the extent that such combinations embrace compounds that are stable compounds (i.e., compounds that can be isolated, characterized, and tested for biological activity). In addition, all subcombinations of the chemical groups listed in the embodiments describing such variables are also specifically embraced by the present disclosure and are disclosed herein just as if each and every such sub-combination of chemical groups was individually and explicitly disclosed herein.

### Compounds

Compounds and salts thereof (such as pharmaceutically acceptable salts) are detailed herein, including in the Summary and in the appended claims. Also provided are the use of all of the compounds described herein, including any and all stereoisomers, including geometric isomers (e.g., cis/trans isomers or E/Z isomers), enantiomers, diastereomers, and mixtures thereof in any ratio including racemic mixtures, salts and solvates of the compounds described herein, as well as methods of making such compounds. Any compound described herein may also be referred to as a drug.

In one aspect, provided are compounds of Formula (I):

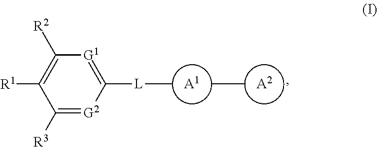

or a tautomer thereof, or a pharmaceutically acceptable salt of any of the foregoing,
wherein
R¹ is R^{1A} and R² is R^{2A}, or R¹ is R^{2A} and R² is R^{1A},
   wherein R^{1A} is —OH, —OPO₃H₂, —OCH₂OPO₃H₂, —OC(O)R^{1A1}, —OC(O)OR^{1A1}, —OC(O)NHR^{1A1}, —OC(O)NR^{1A1}R^{1A2}, or —OR^{1A3},
      wherein R^{1A1} and R^{1A2} are each independently hydrogen, optionally substituted alkyl, optionally substituted alkenyl, optionally substituted alkynyl, optionally substituted cycloalkyl, optionally substituted cycloalkenyl, optionally substituted aryl, optionally substituted heterocyclyl, optionally substituted heteroaryl or —O₀₋₁(CH₂)ₘO(CH₂)ₙOH, wherein m and n are each independently 1 or 2, and;
      R^{1A3} is optionally substituted heteroaryl;
   R^{2A} is —CHO or —CH═NR^{2A1},
      wherein R^{2A1} is optionally substituted heterocyclyl, optionally substituted alkyl, optionally substituted alkenyl, optionally substituted alkynyl, —NR^{2A1A}C(O)R^{2A1B}, —NR^{2A1A}S(O)₂R^{2A1B}, —NR^{2A1A}R^{2A1B}, —OR^{2A1A}, or —NR^{2A1A}C(NR^{2A1B})NR^{2A1C}R^{2A1D}, and
      wherein R^{2A1A}, R^{2A1B}, R^{2A1C}, and R^{2A1D} are each independently hydrogen, optionally substituted alkyl, optionally substituted cycloalkyl, optionally substituted heteroaryl, optionally substituted aryl, or optionally substituted amino; or
   R^{1A} and R^{2A} taken together with

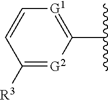

to which they are attached form optionally substituted

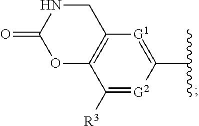

R³ is halo, hydrogen, optionally substituted alkyl, or optionally substituted alkoxy;
G¹ and G² are each independently CH or N;
L is a bond, —C(O)NH—*, —NHC(O)—*, —C(R^{4A})(R^{4B})NHC(O)—*, —C(O)—, —S(O)₂—, —S(O)₂NH—*,

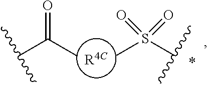

—C(O)N(R^{4D})(CH₂)₂₋₃—*, —C(O)N(CH₃)—*, —(CH₂)OC(O)NH—*, —C(O)NHNH—*, —C(O)NHNHC(O)—*, —CH(R^{4E})NHC(O)O—*, or —C(O)NHO—*,
   wherein R^{4A}, R^{4B}, R^{4D}, and R^{4E} are each independently hydrogen or optionally substituted alkyl,
   R^{4C} is optionally substituted aryl, optionally substituted cycloalkyl, optionally substituted cycloalkenyl, optionally substituted heteroaryl, or optionally substituted heterocyclyl, and
   represents the point of attachment to A¹; and
A¹ and A² are each independently optionally substituted aryl, optionally substituted cycloalkyl, optionally substituted cycloalkenyl, optionally substituted heteroaryl, or optionally substituted heterocyclyl.

In some embodiments, when R¹ is —OH, R³ is fluoro, L is a bond, and A¹ is optionally substituted 5-membered heteroaryl, then A² is not optionally substituted phenyl, optionally substituted pyridinyl, optionally substituted pyrazinyl, or 2,3-dihydrobenzo[b][1,4]dioxin-6-yl; when R¹ is —CHO, R² is —OH, R³ is hydrogen, and L is —C(O), then A¹ is not optionally substituted indolinyl; when L is —C(O)NH—*, then A¹ is not optionally substituted phenyl, optionally substituted pyridinyl, or pyrimidinyl; when R³ is hydrogen, C₁₋₄ alkyl, —CHO, or methoxy, then L is not a bond; and the compound of Formula (I) is not 3-fluoro-5-formyl-4-hydroxy-N-(4-(pyrrolidin-1-yl)phenyl)benzenesulfonamide, 5-(4-(5-fluoropyridin-2-yl)piperazine-1-carbonyl)-2-hydroxy-3-methylbenzaldehyde, 5-(3-(1λ⁴,2λ²,4-triazol-1-yl)azetidine-1-carbonyl)-2-hydroxy-3-methylbenzaldehyde, tert-butyl (3-(1-(3-formyl-4-hydroxybenzoyl)piperidin-4-yl)benzyl)carbamate, 5-(4-cyclopropyl-3-oxopiperazine-1-carbonyl)-2-hydroxy-3-methylbenzaldehyde, 2-(5-((((4-formyl-3-hydroxybenzyl)oxy)carbonyl)amino)benzo[d]thiazol-2-yl)-4,5-dihydrothiazole-4-carboxylic acid, 4-formyl-3-hydroxybenzyl (6-(benzo[d]oxazol-2-yl)naphthalen-2-yl)carbamate, 5-[2-(3,4-diethoxyphenyl)-4-thiazolyl]-3-formyl-2-hydroxy-benzoic acid methyl ester, or a salt of any of the foregoing.

In one aspect, provided are compounds of Formula (I-1):

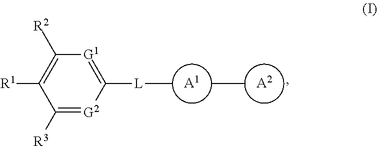

or a tautomer thereof, or a pharmaceutically acceptable salt of any of the foregoing,
wherein
R¹ is R^{1A} and R² is R^{2A}, or R¹ is R^{2A} and R² is R^{1A},
   wherein R^{1A} is —OH, —OPO₃H₂, —OCH₂OPO₃H₂, —OC(O)R^{1A1}, —OC(O)OR^{1A1}, —OC(O)NHR^{1A1}, or —OC(O)NR^{1A1}R^{1A2},
      wherein R^{1A1} and R^{1A2} are each independently hydrogen, optionally substituted alkyl, optionally substituted alkenyl, optionally substituted alkynyl, optionally substituted cycloalkyl, optionally substituted cycloalkenyl, optionally substituted aryl, optionally substituted heterocyclyl, optionally substituted heteroaryl, or —O₀₋₁(CH₂)ₘO(CH₂)ₙOH, wherein m and n are each independently 1 or 2, and
   R^{2A} is —CHO or —CH═NR^{2A1},
      wherein R^{2A1} is optionally substituted heterocyclyl, optionally substituted alkyl, optionally substituted alkenyl, optionally substituted alkynyl, —NR^{2A1A}C(O)R^{2A1B}, —NR^{2A1A}S(O)₂R^{2A1B}, —NR^{2A1A}R^{2A1B}, —OR^{2A1A} or —NR^{2A1A}C(NR^{2A1B})NR^{2A1C}R^{2A1D}, and
      wherein R^{2A1A}, R^{2A1B}, R^{2A1C}, and R^{2A1D} are each independently hydrogen, optionally substituted alkyl, optionally substituted cycloalkyl, or optionally substituted heteroaryl;
R³ is halo, hydrogen, optionally substituted alkyl, or optionally substituted alkoxy;
G¹ and G² are each independently CH or N;
L is a bond, —C(O)NH—*, —NHC(O)—*, —C(R^{4A})(R^{4B})NHC(O)—*, —C(O)—, —S(O)₂—, —S(O)₂NH—*, or

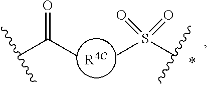

wherein R^{4A} and R^{4B} are each independently hydrogen or optionally substituted alkyl,
R^{4C} is optionally substituted aryl, optionally substituted cycloalkyl, optionally substituted cycloalkenyl, optionally substituted heteroaryl, or optionally substituted heterocyclyl, and
represents the point of attachment to A¹; and
A¹ and A² are each independently optionally substituted aryl, optionally substituted cycloalkyl, optionally substituted cycloalkenyl, optionally substituted heteroaryl, or optionally substituted heterocyclyl.

In some embodiments of Formula (I), or a tautomer thereof, or a pharmaceutically acceptable salt of any of the foregoing, when R¹ is —OH, R³ is fluoro, L is a bond, and A¹ is optionally substituted 5-membered heteroaryl, then A² is not optionally substituted phenyl, optionally substituted pyridinyl, optionally substituted pyrazinyl, or 2,3-dihydrobenzo[b][1,4]dioxin-6-yl. In some variations, when L is a bond, and A¹ is pyrazolyl, then A² is not optionally substituted phenyl, tetrahydropyranyl, 2,3-dihydrobenzo[b][1,4]dioxin-6-yl, or pyrazinyl. In some variations, when L is a bond, and A¹ is optionally substituted thiazolyl, then A² is not optionally substituted phenyl or optionally substituted pyridinyl. In some variations, when L is a bond, and A¹ is 1,2,3-triazolyl, 1,2,4-triazolyl, thiophenyl, oxazolyl, isoxazolyl, isothiazolyl, 1,2,4-oxadiazolyl, 1,3,4-oxadiazolyl, 1,2,4-thiadiazolyl, 1,3,4-thiadiazolyl, or tetrazolyl, then A² is not optionally substituted phenyl. In some variations, when L is a bond, and A¹ is 5-membered heteroaryl, then A² is not optionally substituted phenyl.

In some embodiments of Formula (I), or a tautomer thereof, or a pharmaceutically acceptable salt of any of the foregoing, when L is —C(O)NH—*, then A¹ is not optionally substituted phenyl, optionally substituted pyridinyl, or pyrimidinyl. In some variations, when L is —S(O)₂NH—*, then A¹ is not phenyl. In some variations, the compound of Formula (I) is not 3-fluoro-5-formyl-4-hydroxy-N-(4-(pyrrolidin-1-yl)phenyl)benzenesulfonamide or a salt thereof.

In some embodiments of Formula (I), or a tautomer thereof, or a pharmaceutically acceptable salt of any of the foregoing, when R³ is hydrogen, methyl, isobutyl, or methoxy, then L is not a bond. In some variations, when R³ is hydrogen, optionally substituted alkyl, or optionally substituted alkoxy, then L is not a bond. In some variations, when R³ is hydrogen, C₁₋₄ alkyl, —CHO, or methoxy, then L is not a bond. In some variations, R³ is not —CHO. In some variations, when R³ is alkyl substituted with oxo, then L is not a bond. In some variations, when R³ is substituted with oxo, then L is not a bond. In some variations, R³ is not alkyl substituted with oxo. In some variations, R³ is not substituted with oxo.

In some embodiments of Formula (I), or a tautomer thereof, or a pharmaceutically acceptable salt of any of the foregoing, the compound of Formula (I) is not 5-(4-(5-fluoropyridin-2-yl)piperazine-1-carbonyl)-2-hydroxy-3-methylbenzaldehyde, 5-(3-(1λ⁴,2λ²,4-triazol-1-yl)azetidine-1-carbonyl)-2-hydroxy-3-methylbenzaldehyde, tert-butyl (3-(1-(3-formyl-4-hydroxybenzoyl)piperidin-4-yl)benzyl)carbamate, or 5-(4-cyclopropyl-3-oxopiperazine-1-carbonyl)-2-hydroxy-3-methylbenzaldehyde, or a salt of any of the foregoing. In some variations, when R¹ is —OH, R² is —CHO, R³ is hydrogen or methyl, and L is —C(O)—, then A¹ is not azetidinyl, piperidinyl, or optionally substituted piperazinyl. In some variations, when R¹ is —OH, R² is —CHO, R³ is hydrogen, alkyl, or alkoxy, and L is —C(O)—, then A¹ is not optionally substituted heterocyclyl. In some variations, when L is —C(O)—, then A¹ is not azetidinyl, piperidinyl, or optionally substituted piperazinyl. In some variations, when L is —C(O)—, then A¹ is not optionally substituted heterocyclyl.

In some embodiments of Formula (I), or a tautomer thereof, or a pharmaceutically acceptable salt of any of the foregoing, the compound of Formula (I) is not 2-(5-((((4-formyl-3-hydroxybenzyl)oxy)carbonyl)amino)benzo[d]thiazol-2-yl)-4,5-dihydrothiazole-4-carboxylic acid, 4-formyl-3-hydroxybenzyl (6-(benzo[d]oxazol-2-yl)naphthalen-2-yl)carbamate, or a salt of any of the foregoing. In some variations, when R¹ is —CHO and R³ is H, then L is not —CH₂OC(O)NH—*. In some variations, when L is CH₂OC(O)NH—*, then R¹ is not —CHO or R³ is not H. In some variations, when R¹ is —CHO, then L is not —CH₂OC(O)NH—*. In some variations, when R³ is H, then L is not —CH₂OC(O)NH—*.

In some embodiments of Formula (I), or a tautomer thereof, or a pharmaceutically acceptable salt of any of the foregoing, the compound of Formula (I) is not 5-[2-(3,4-diethoxyphenyl)-4-thiazolyl]-3-formyl-2-hydroxy-benzoic acid methyl ester or a salt thereof. In some variations, when R³ is alkyl substituted with oxo, then L is not a bond. In some variations, when R³ is substituted with oxo, then L is not a bond. In some variations, R³ is not alkyl substituted with oxo. In some variations, R³ is not substituted with oxo.

In some embodiments of Formula (I), or a tautomer thereof, or a pharmaceutically acceptable salt of any of the foregoing, the compound of Formula (I) is not 3-fluoro-5-formyl-4-hydroxy-N-(4-(pyrrolidin-1-yl)phenyl)benzenesulfonamide, 5-(4-(5-fluoropyridin-2-yl)piperazine-1-carbonyl)-2-hydroxy-3-methylbenzaldehyde, 5-(3-(1λ⁴,2λ²,4-triazol-1-yl)azetidine-1-carbonyl)-2-hydroxy-3-methylbenzaldehyde, tert-butyl (3-(1-(3-formyl-4-hydroxybenzoyl)piperidin-4-yl)benzyl)carbamate, 5-(4-cyclopropyl-3-oxopiperazine-1-carbonyl)-2-hydroxy-3-methylbenzaldehyde, 2-(5-((((4-formyl-3-hydroxybenzyl)oxy)carbonyl)amino)benzo[d]thiazol-2-yl)-4,5-dihydrothiazole-4-carboxylic acid, 4-formyl-3-hydroxybenzyl (6-(benzo[d]oxazol-2-yl)naphthalen-2-yl)carbamate, 5-[2-(3,4-diethoxyphenyl)-4-thiazolyl]-3-formyl-2-hydroxy-benzoic acid methyl ester, or a salt of any of the foregoing.

In some embodiments of Formula (I), or a tautomer thereof, or a pharmaceutically acceptable salt of any of the foregoing, the compound of Formula (I) is not 4,4′-[(2,2′,3,3′-tetrahydro[4,4′-bi-1H-indole]-1,1′-diyl)dicarbonyl]bis[2-hydroxy-benzaldehyde], 4-[[4-(2,3-dihydro-1,4-benzodioxin-6-yl)-2,3-dihydro-1H-indol-1-yl]carbonyl]-2-hydroxy-benzaldehyde, or a salt of any of the foregoing. In some variations, when R¹ is —CHO, R² is —OH, R³ is hydrogen, and L is —C(O), then A¹ is not optionally substituted indolinyl.

In some embodiments of Formula (I), or a tautomer thereof, or a pharmaceutically acceptable salt of any of the foregoing, R¹ is R^{1A} and R² is R^{2A}. In some variations, R¹ is R^{2A} and R² is R^{1A}.

In some embodiments of Formula (I), or a tautomer thereof, or a pharmaceutically acceptable salt of any of the foregoing, R¹ is —OH. In some variations, R¹ is —OPO₃H₂. In some variations, R¹ is —OCH₂OPO₃H₂. In some variations, R¹ is —OC(O)R^{1A1} or

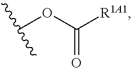

In some variations, R¹ is —OC(O)OR^{1A1} or

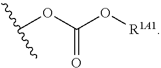

In some variations, R¹ is —OC(O)NHR^{1A1} or

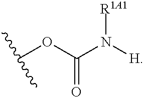

In some variations, R¹ is —OC(O)NR^{1A1}R^{1A2} or

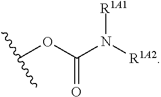

In some variations, R¹ is —OR^{1A3} or

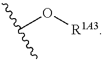

In some embodiments of Formula (I), or a tautomer thereof, or a pharmaceutically acceptable salt of any of the foregoing, R² is —OH. In some variations, R² is —OPO₃H₂. In some variations, R² is —OCH₂OPO₃H₂. In some variations, R² is —OC(O)R^{1A1} or

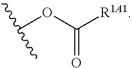

In some variations, R² is —OC(O)OR^{1A1} or

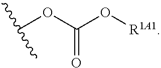

In some variations, R² is —OC(O)NHR^{1A2} or

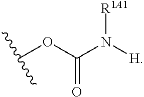

In some variations, R² is —OC(O)NR^{1A1}R^{1A2} or

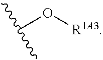

In some variations, R² is —OR^{1A3} or

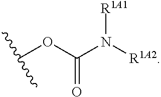

In some embodiments of Formula (I), or a tautomer thereof, or a pharmaceutically acceptable salt of any of the foregoing, R^{1A1} is hydrogen. In some variations, R^{1A1} is optionally substituted alkyl. In some variations, R^{1A1} is optionally substituted C₁-C₁₀ alkyl. In some variations, R^{1A1} is methyl, ethyl, propyl, butyl, pentyl, or hexyl, each of which is optionally substituted. In some variations, R^{1A1} is optionally substituted alkenyl. In some variations, R^{1A1} is optionally substituted alkynyl. In some variations, R^{1A1} is optionally substituted cycloalkyl. In some variations, R^{1A1} is C₃-C₁₀ cycloalkyl. In some variations, R^{1A1} is cyclopentyl or cyclohexyl. In some variations, R^{1A1} is optionally substituted cycloalkenyl. In some variations, R^{1A1} is optionally substituted aryl. In some variations, R^{1A1} is C₆-C₁₀ aryl. In some variations, R^{1A1} is phenyl. In some variations, R^{1A1} is optionally substituted heterocyclyl. In some variations, R^{1A1} is optionally substituted heteroaryl. In some variations, R^{1A1} is -(optionally substituted alkyl)(optionally substituted cycloalkyl). In some variations, R^{1A1} is -(optionally substituted alkyl)(optionally substituted aryl). In some variations, R^{1A} is -(optionally substituted alkyl)(optionally substituted heterocyclyl). In some variations, R^{1A1} is -(optionally substituted alkyl)(optionally substituted heteroaryl). In some variations, R^{1A1} is —O₀₋₁(CH₂)ₘO(CH₂)ₙOH, wherein m and n are each independently 1 or 2. In some variations, R^{1A1} is

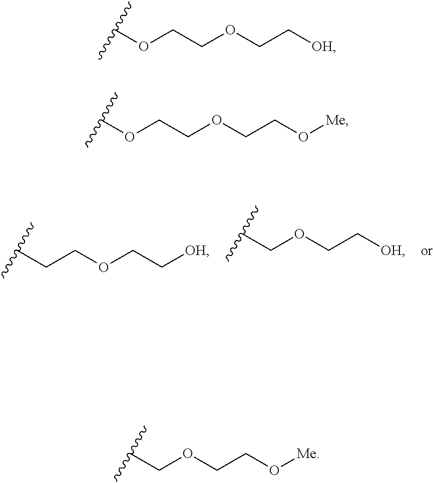 [SMILES: *OCCOCCO ; *OCCOCCOC ; *COCCO ; *CCOCCO ; "or" ; *COCCOC]

In some variations, R^{1A1} is —(C₁₋₄ alkyl)(optionally substituted heterocyclyl). In some variations, R^{1A1} is —(CH₂)₀₋₃O(CH₂)₀₋₃O(CH₂)₀₋₃. In some variations, R^{1A1} is —P(O)(OR^{1a1})(OR^{1a2}). In some variations, R^{1a1} and R^{1a2} are each independently hydrogen or C₁₋₆ alkyl.

In some embodiments of Formula (I), or a tautomer thereof, or a pharmaceutically acceptable salt of any of the foregoing, R^{1A1} is optionally substituted with one or more substituents independently selected from the group consisting of —OH, halo, and optionally substituted C₁₋₆ alkyl. In some variations, R^{1A1} is C₁₋₆ alkyl optionally substituted with one or more substituents independently selected from the group consisting of C₁₋₆ alkoxy optionally substituted with one or more substituents independently selected from the group consisting of —OH and C₁₋₄ alkoxy; —COOH; amino; —OH; C₆₋₁₄ aryl optionally substituted with one or more —OH; 4- to 14-membered heterocyclyl optionally substituted with one or more C₁₋₆ alkyl; —C(O)NH(C₁₋₆ alkyl) optionally substituted with one or more substituents independently selected from the group consisting of —COOH and amino; —NHC(O)(C₁₋₆ alkyl) optionally substituted with one or more substituents independently selected from the group consisting of —COOH and amino; and —P(O)(OC₁₋₆ alkyl)(OC₁₋₆ alkyl).

In some embodiments of Formula (I), or a tautomer thereof, or a pharmaceutically acceptable salt of any of the foregoing, R^{1A2} is hydrogen. In some variations, R^{1A2} is optionally substituted alkyl. In some variations, R^{1A2} is optionally substituted C₁-C₁₀ alkyl. In some variations, R^{1A2} is methyl, ethyl, propyl, butyl, pentyl, or hexyl, each of which is optionally substituted. In some variations, R^{1A2} is optionally substituted alkenyl. In some variations, R^{1A2} is optionally substituted alkynyl. In some variations, R^{1A2} is optionally substituted cycloalkyl. In some variations, R^{1A2} is C₃-C₁₀ cycloalkyl. In some variations, R^{1A2} is cyclopentyl or cyclohexyl. In some variations, R^{1A2} is optionally substituted cycloalkenyl. In some variations, R^{1A2} is optionally substituted aryl. In some variations, R^{1A2} is C₆-C₁₀ aryl. In some variations, R^{1A2} is phenyl. In some variations, R^{1A2} is optionally substituted heterocyclyl. In some variations, R^{1A2} is optionally substituted heteroaryl. In some variations, R^{1B} is -(optionally substituted alkyl)(optionally substituted cycloalkyl). In some variations, R^{1A2} is -(optionally substituted alkyl)(optionally substituted aryl). In some variations, R^{1A2} is -(optionally substituted alkyl)(optionally substituted heterocyclyl). In some variations, R^{1A2} is -(optionally substituted alkyl)(optionally substituted heteroaryl). In some variations, R^{1A2} is —O₀₋₁(CH₂)ₘO(CH₂)ₙOH, wherein m and n are each independently 1 or 2. In some variations, R^{1A2} is

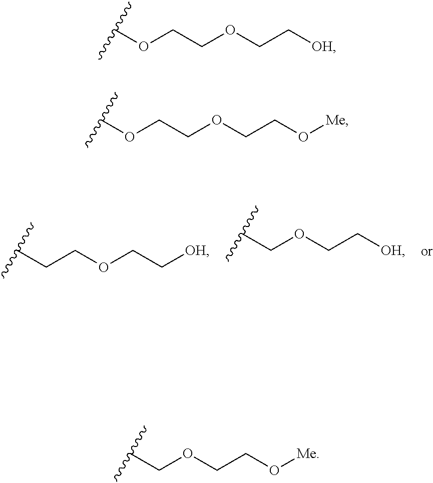 [SMILES: *OCCOCCO ; *OCCOCCOC ; *COCCO ; *CCOCCO ; "or" ; *COCCOC]

In some variations, R^{1A1} is —(C₁₋₄ alkyl)(optionally substituted heterocyclyl). In some variations, R^{1A1} is —(CH₂)₀₋₃O(CH₂)₀₋₃O(CH₂)₀₋₃. In some variations, R^{1A1} is —P(O)(OR^{1a1})(OR^{1a2}). In some variations, R^{1a1} and R^{1a2} are each independently hydrogen or C₁₋₆ alkyl.

In some embodiments of Formula (I), or a tautomer thereof, or a pharmaceutically acceptable salt of any of the foregoing, R^{1A2} is optionally substituted with one or more substituents independently selected from the group consisting of —OH, halo, and optionally substituted C₁₋₆ alkyl. In some variations, R^{1A2} is C₁₋₆ alkyl optionally substituted with one or more substituents independently selected from the group consisting of C₁₋₆ alkoxy optionally substituted with one or more substituents independently selected from the group consisting of —OH and C₁₋₄ alkoxy; —COOH; amino; —OH; C₆₋₁₄ aryl optionally substituted with one or more —OH; —C(O)NH(C₁₋₆ alkyl) optionally substituted with one or more substituents independently selected from the group consisting of —COOH and amino; —NHC(O)(C₁₋₆ alkyl) optionally substituted with one or more substituents independently selected from the group consisting of —COOH and amino; and —P(O)(OC₁₋₆ alkyl)(OC₁₋₆ alkyl).

In some embodiments of Formula (I), or a tautomer thereof, or a pharmaceutically acceptable salt of any of the foregoing, R^{1A3} is optionally substituted heteroaryl. In some variations, R^{1A3} is heteroaryl optionally substituted with C₁₋₆ alkyl. In some variations, R^{1A3} is

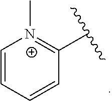 [SMILES: *c1cccc[n+]1C]

In some variations, R^{1A3} is —PO₃H₂, —P(O)H(OC₁₋₆ alkyl), or —P(O)(OC₁₋₆ alkyl)(OC₁₋₆ alkyl).

In some embodiments of Formula (I), or a tautomer thereof, or a pharmaceutically acceptable salt of any of the foregoing, R¹ is

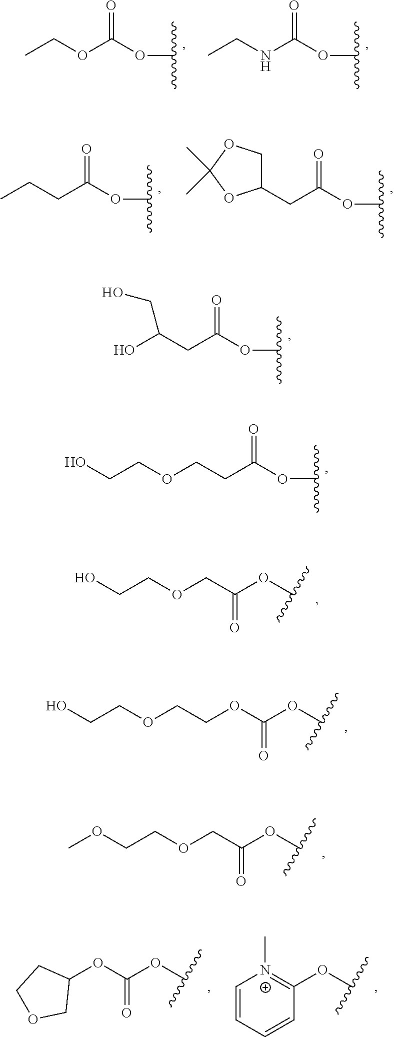

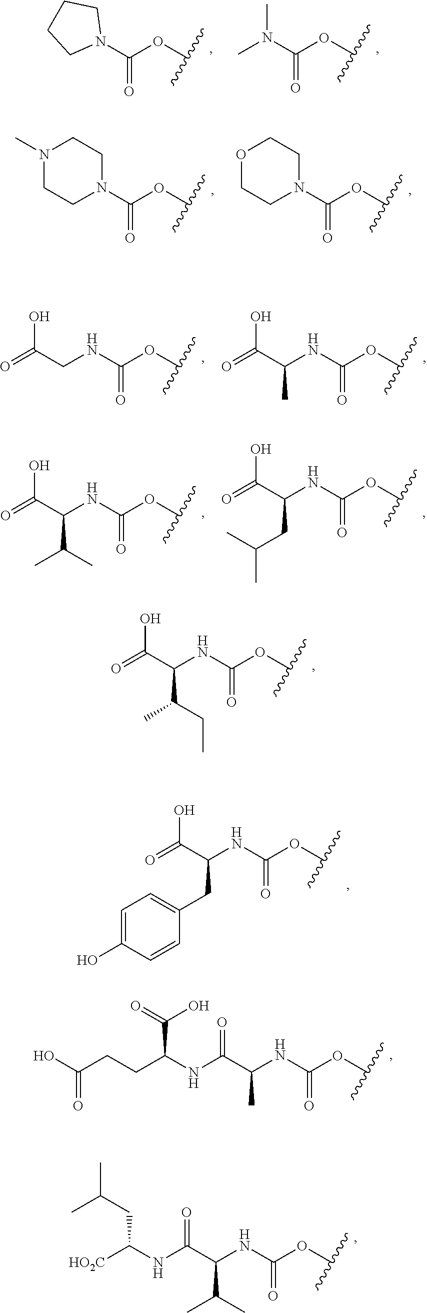

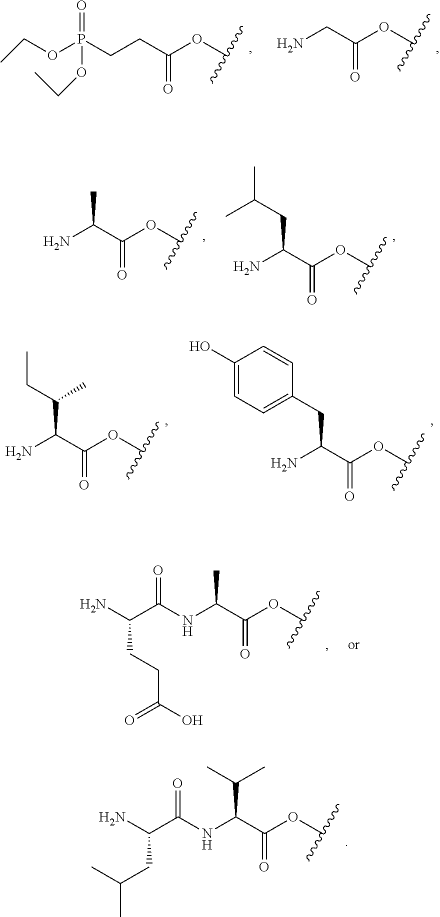

In some embodiments of Formula (I), or a tautomer thereof, or a pharmaceutically acceptable salt of any of the foregoing, R² is

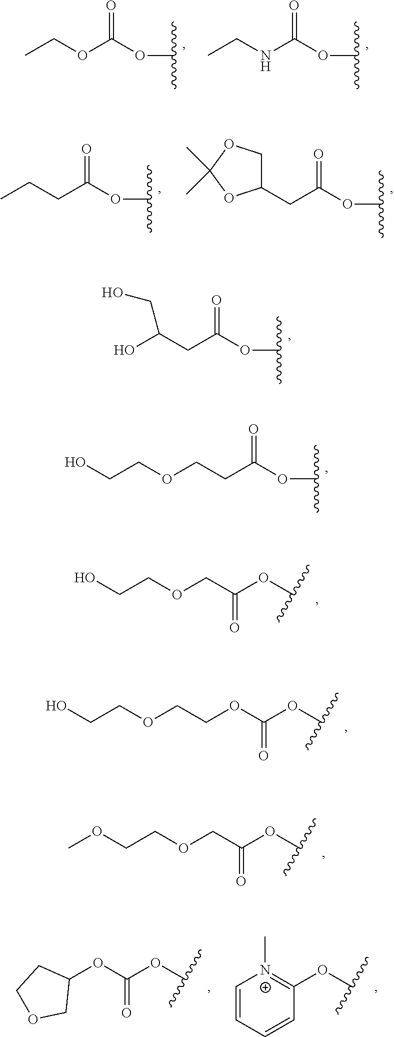

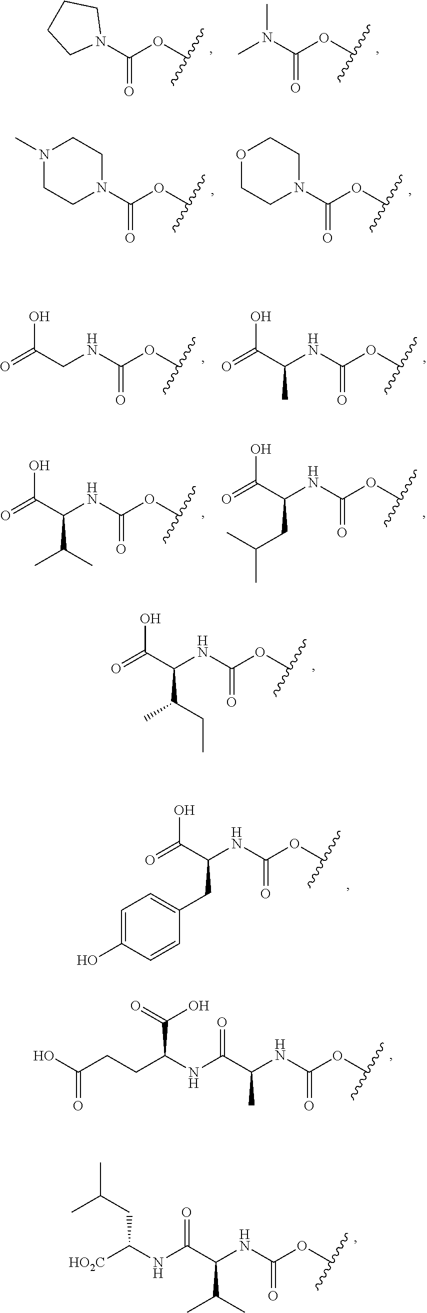

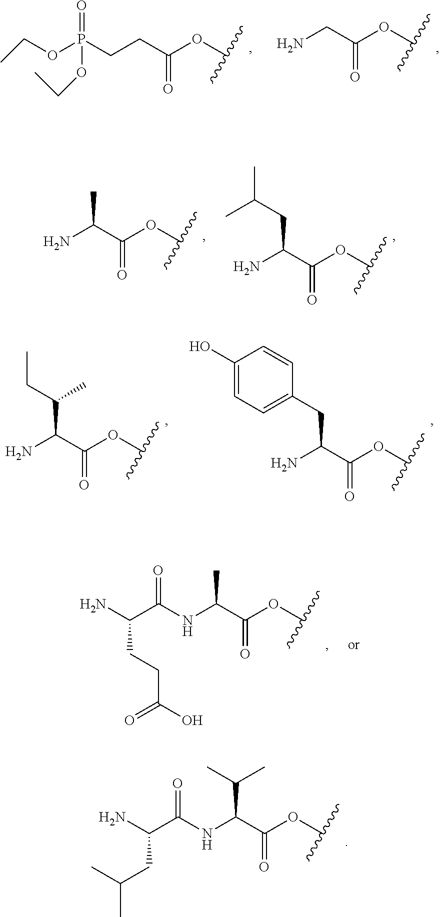

In some embodiments of Formula (I), or a tautomer thereof, or a pharmaceutically acceptable salt of any of the foregoing, R² is —CHO. In some variations, R¹ is —CHO. In some variations, R² is —CH═NR^{2A1}. In some variations, R¹ is —CH═NR^{2A1}. In some variations, R^{2A1} is optionally substituted heterocyclyl. In some variations, R^{2A1} is optionally substituted alkyl. In some variations, R^{2A1} is optionally substituted alkenyl. In some variations, R^{2A1} is optionally substituted alkynyl. In some variations, R^{2A1} is —NR^{2A1A}C(O)R^{2A1}B. In some variations, R^{2A1} is —NR^{2A1A}S(O)₂R^{2A1B}. In some variations, R^{2A1} is —NR^{2A1A}R^{2A1B}. In some variations, R^{2A1} is —OR^{2A1A}. In some variations, R^{2A1} is —NR^{2A1A}C(NR^{2A1B})NR^{2A1C}R^{2A1D}. In some variations, R^{2A1A}, R^{2A1B}, R^{2A1C}, and R^{2A1D} are each independently hydrogen, optionally substituted alkyl, optionally substituted cycloalkyl, optionally substituted heteroaryl, optionally substituted aryl, or optionally substituted amino. In some variations, R^{2A1} is

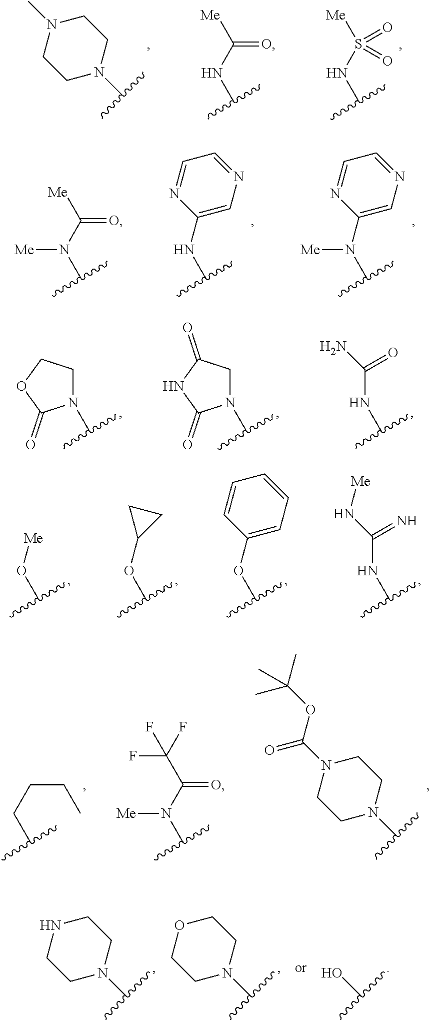

In some embodiments of Formula (I), or a tautomer thereof, or a pharmaceutically acceptable salt of any of the foregoing, R^{2A1} is optionally substituted with one or more substituents independently selected from the group consisting of C₁₋₆ alkyl and oxo.

In some embodiments of Formula (I), or a tautomer thereof, or a pharmaceutically acceptable salt of any of the foregoing, R^{2A1A}, R^{2A1B}, R^{2A1C}, and R^{2A1D} are each independently and optionally substituted with one or more halo.

In some embodiments of Formula (I), or a tautomer thereof, or a pharmaceutically acceptable salt of any of the foregoing, R^{1A} and R^{2A} taken together with

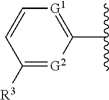

to which they are attached form optionally substituted

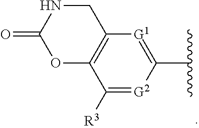

In some variations,

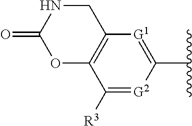

is optionally substituted with one or more substituents independently selected from the group consisting of fluoro, —OH, and C₁₋₆ alkyl optionally substituted with one or more substituents independently selected from the group consisting of C₁₋₆ alkoxy and oxo. In some variations, R^{1A} and R^{2A} taken together with

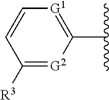

to which they are attached form

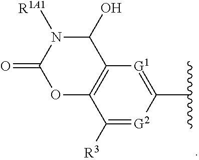

In some embodiments of Formula (I), or a tautomer thereof, or a pharmaceutically acceptable salt of any of the foregoing, R³ is halo. In some variations, R³ is fluoro. In some variations, R³ is chloro. In some variations, R³ is bromo. In some variations, R³ is iodo. In some variations, R³ is hydrogen. In some variations, R³ is optionally substituted alkyl. In some variations, R³ is methyl. In some variations, R³ is optionally substituted alkoxy. In some variations, R³ is methoxy. In some variations, R³ is halo, optionaly substituted C₁₋₆ alkyl, or optionally substituted C₁₋₆ alkoxy.

In some embodiments of Formula (I), or a tautomer thereof, or a pharmaceutically acceptable salt of any of the foregoing, G¹ and G² are both CH. In some variations, G¹ and G² are both N. In some variations, one of G¹ and G² is CH and the other is N. In some variations, G¹ is CH and G² is N. In some variations, G¹ is N and G² is CH.

In some embodiments of Formula (I), or a tautomer thereof, or a pharmaceutically acceptable salt of any of the foregoing, L is a bond. In some variations, L is —C(O)NH—*. In some variations, L is —NHC(O)—*. In some variations, L is —C(R^{4A})(R^{4B})NHC(O)—*. In some variations, L is —C(O)N(R^{4D})(CH₂)₂₋₃—*. In some variations, L is —C(O)N(R^{4D})(CH₂)₀₋₃—*. In some variations, L is —C(O)N(CH₃)—*. In some variations, L is —(CH₂)OC(O)NH—*. In some variations, L is —C(O)NHNH—*. In some variations, L is —C(O)NHNHC(O)—*. In some variations, L is —CH(R^{4E})NHC(O)O—*. In some variations, L is —C(O)NHO—*.

In some embodiments, R^{4A}, R^{4B}, R^{4D}, and R^{4E} are each independently hydrogen or optionally substituted alkyl. In some variations, R^{4A}, R^{4B}, R^{4D}, and R^{4E} are each independently hydrogen or optionally substituted C₁₋₆ alkyl. In some variations, R^{4A} and R^{4B} are both hydrogen. In some variations, R^{4A} and R^{4B} are both optionally substituted C₁-C₁₀ alkyl. In some variations, R^{4A} and R^{4B} are both methyl. In some variations, one of R^{4A} and R^{4B} is hydrogen and the other is optionally substituted C₁-C₁₀ alkyl.

In some embodiments of Formula (I), or a tautomer thereof, or a pharmaceutically acceptable salt of any of the foregoing, L is —CH(R^{4F})OC(O)NH—*, wherein R^{4F} is hydrogen, C₁₋₆ alkyl, or C₁₋₆ haloalkyl.

In some variations, L is —C(O)—. In some variations, L is —S(O)₂—. In some variations, L is —S(O)₂NH—*. In some variations, L is

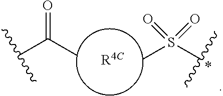

In some variations, R^{4C} is optionally substituted aryl. In some variations, R^{4C} is optionally substituted cycloalkyl. In some variations, R^{4C} is optionally substituted cycloalkenyl. In some variations, R^{4C} is optionally substituted heteroaryl. In some variations, R^{4C} is optionally substituted heterocyclyl. In some variations, R^{4C} is azetidinyl or piperidinyl.

It will be understood that * used in the variations of L denotes the point of attachment to A¹. For example, when L is —C(O)NH—*, then the corresponding structure of Formula (I) is

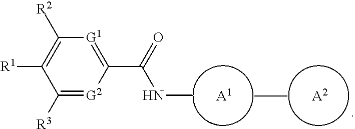

In some embodiments of Formula (I), or a tautomer thereof, or a pharmaceutically acceptable salt of any of the foregoing, L is

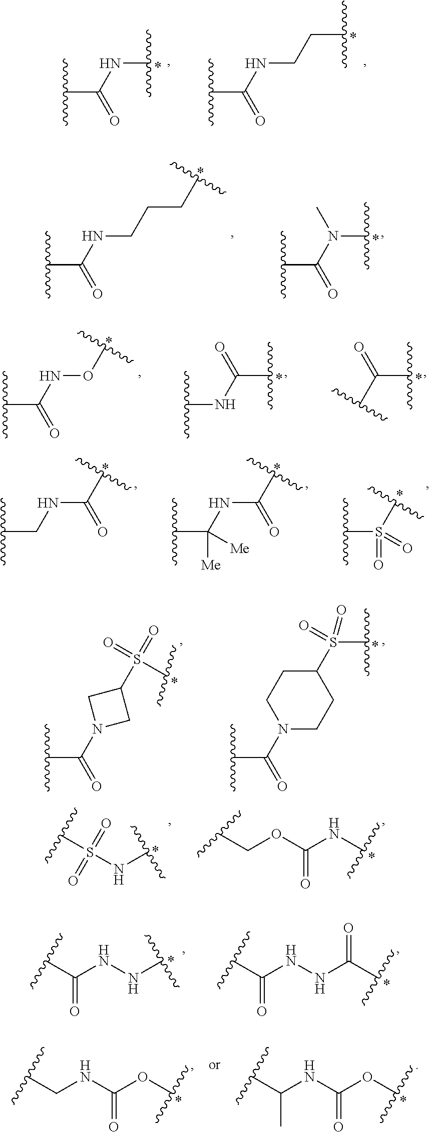

In some embodiments of Formula (I), or a tautomer thereof, or a pharmaceutically acceptable salt of any of the foregoing, A¹ is optionally substituted aryl. In some variations, A¹ is optionally substituted phenyl. In some variations, A¹ is optionally substituted cycloalkyl. In some variations, A¹ is cyclobutyl. In some variations, A¹ is optionally substituted cycloalkenyl. In some variations, A¹ is optionally substituted heteroaryl. In some variations, A¹ is thiazolyl, oxazolyl, pyrazolyl, thiadiazolyl, oxadiazolyl, pyridinyl, pyridazinyl, pyrimidinyl, pyrazinyl, or benzothiazolyl, each of which is optionally substituted. In some variations, A¹ is optionally substituted heterocyclyl. In some variations, A¹ is azetidinyl, pyrrolidinyl, piperidinyl, piperazinyl, diazepane, or tetrahydrothienopyridine, each of which is optionally substituted. In some variations, A¹ is optionally substituted thiazolyl. In some variations, A¹ is optionally substituted thiadiazolyl. In some variations, A¹ is optionally substituted pyrazolyl. In some variations, A¹ is optionally substituted piperidinyl. In some variations, A¹ is

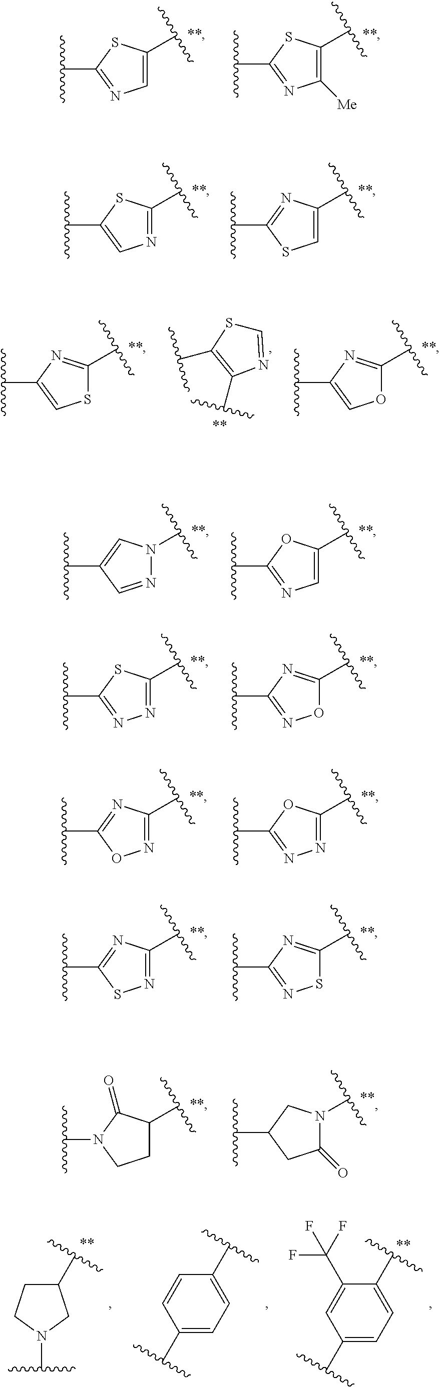

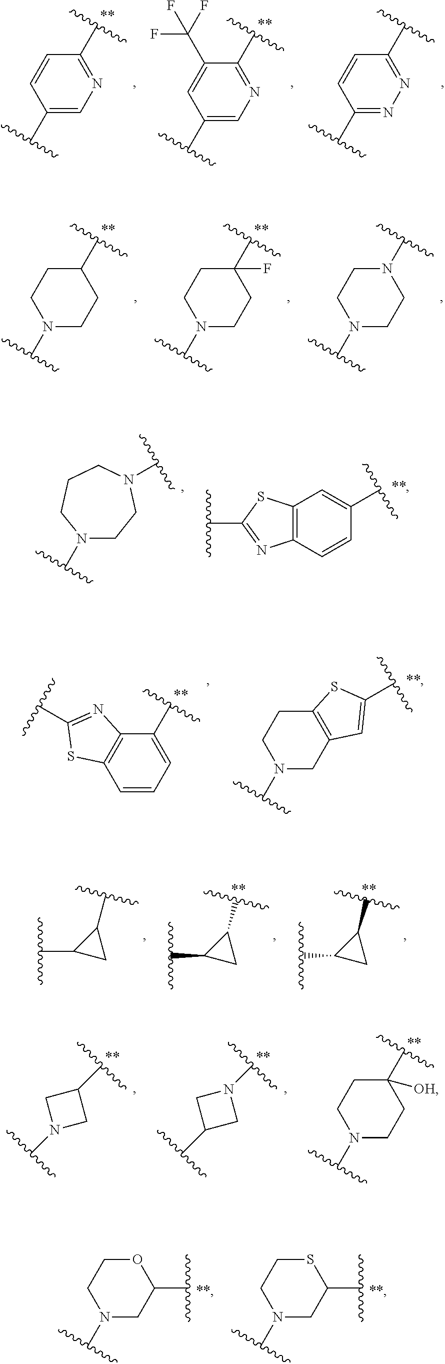

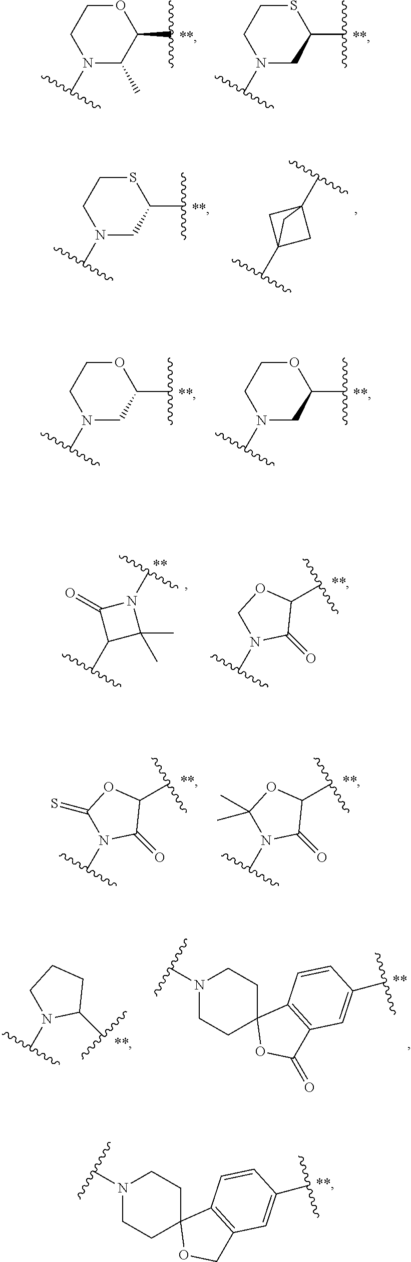

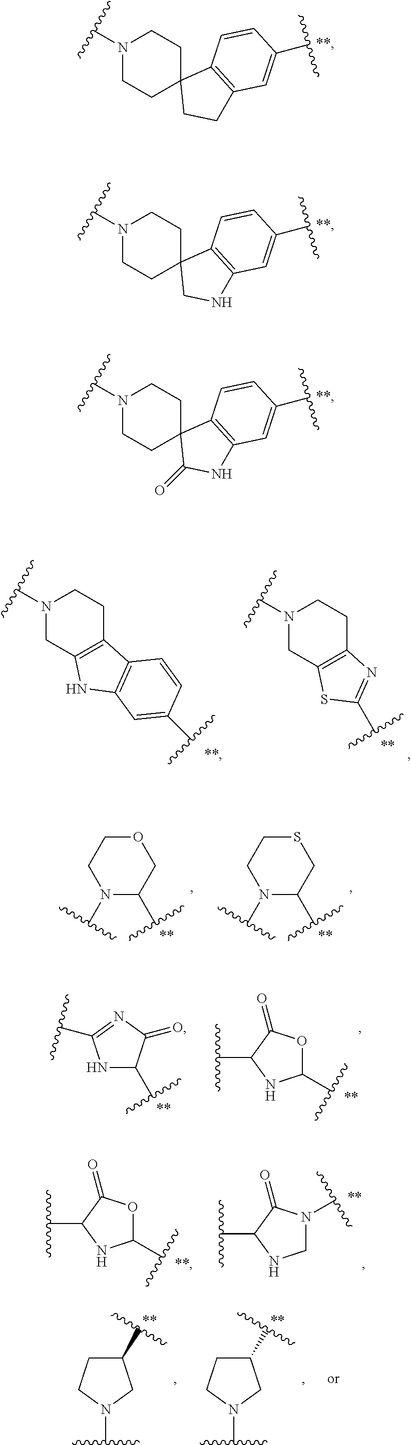

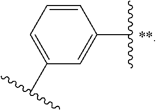 [SMILES: *c1cccc(*)c1]

In some embodiments, A¹ is optionally substituted with one or more substituents independently selected from the group consisting of halo, oxo, ═S, cyano, —OH, —SO₂(C₁₋₆ alkyl), —SO₂NH(C₁₋₆ alkyl), —SO₂NH₂, —NHSO₂(C₁₋₆ alkyl), C₃₋₁₀ cycloalkyl, C₁₋₆ alkoxy, C₁₋₆ alkyl optionally substituted with one or more halo, 4- to 14-membered heterocyclyl optionally substituted with one or more halo, 4- to 14-membered heteroaryl optionally substituted with one or more C₁₋₆ alkyl or C₃₋₁₀ cycloalkyl. In some variations, A¹ is optionally substituted with C₁₋₆ alkyl, C₁₋₆ alkoxy, C₃₋₁₀ cycloalkyl, C₆₋₁₄ aryl, 4- to 14-membered heterocyclyl, or 4- to 14-membered heteroaryl, each optionally substituted.

It will be understood that ** used in the variations of A¹ denotes the point of attachment to A². For example, when A¹ is

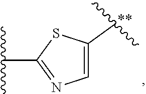 [SMILES: *c1cnc(*)s1]

then the corresponding structure of Formula (I) is

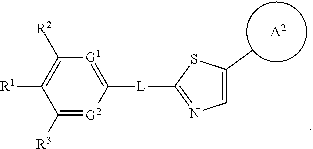

In some variations, A² is optionally substituted aryl. In some variations, A² is optionally substituted cycloalkyl. In some variations, A² is optionally substituted cycloalkenyl. In some variations, A² is optionally substituted heteroaryl. In some variations, A² is optionally substituted heterocyclyl. In some variations, A² is pyrrolidinyl, piperidinyl, piperazinyl, phenyl, pyridinyl, pyridazinyl, pyrimidinyl, or pyrazinyl, each of which is optionally substituted. In some variations, A² is optionally substituted piperidinyl. In some variations, A² is optionally substituted phenyl. In some variations, A² is optionally substituted piperazinyl. In some variations, A² is optionally substituted pyrrolidinyl. In some variations, A² is optionally substituted pyridinyl. In some variations, A² is

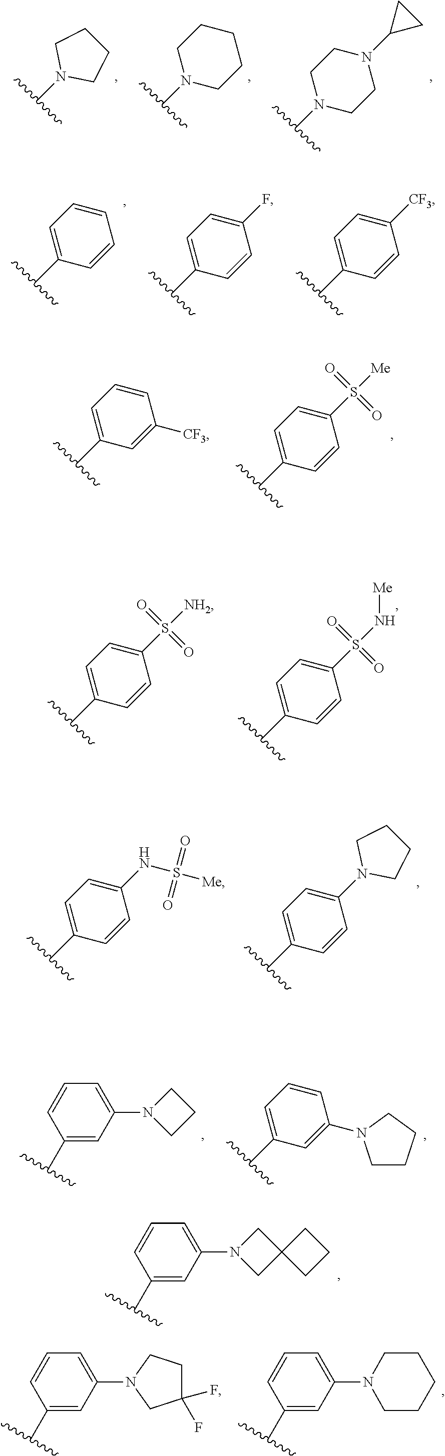

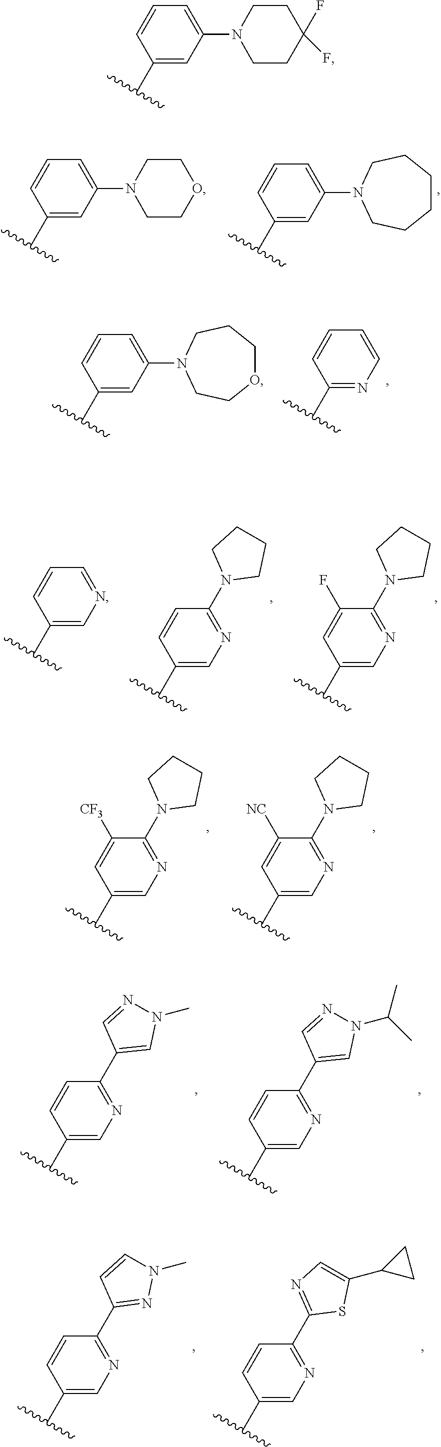

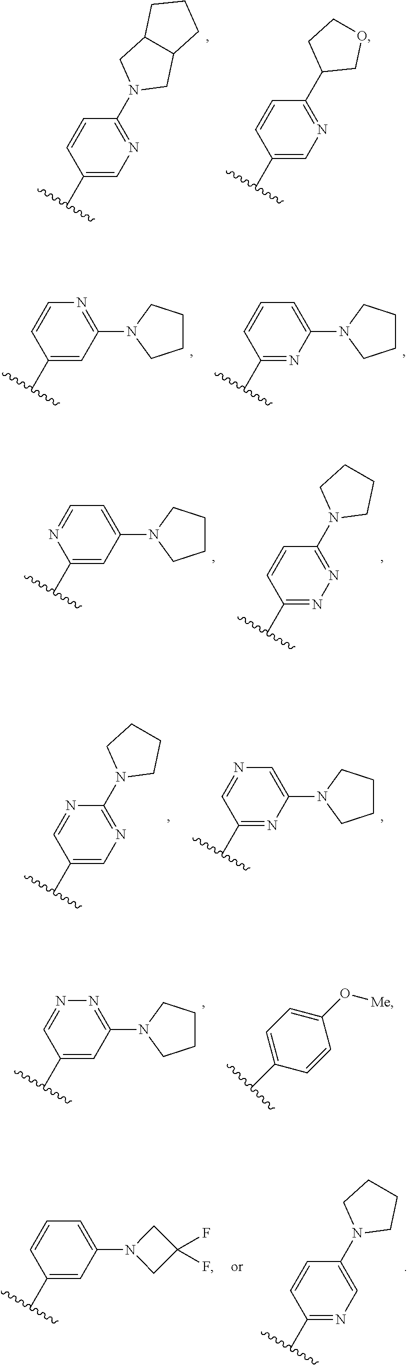

In some embodiments, A² is optionally substituted with one or more substituents independently selected from the group consisting of halo, oxo, ═S, cyano, —OH, —SO₂(C₁₋₆ alkyl), —SO₂NH(C₁₋₆ alkyl), —SO₂NH₂, —NHSO₂(C₁₋₆ alkyl), C₃₋₁₀ cycloalkyl, C₁₋₆ alkoxy, C₁₋₆ alkyl optionally substituted with one or more halo, 4- to 14-membered heterocyclyl optionally substituted with one or more halo, 4- to 14-membered heteroaryl optionally substituted with one or more C₁₋₆ alkyl or C₃₋₁₀ cycloalkyl. In some variations, A² is optionally substituted with C₁_6 alkyl, C₁₋₆ alkoxy, C₃₋₁₀ cycloalkyl, C₆₋₁₄ aryl, 4- to 14-membered heterocyclyl, or 4- to 14-membered heteroaryl, each optionally substituted.

In some embodiments of Formula (I), or a tautomer thereof, or a pharmaceutically acceptable salt of any of the foregoing,

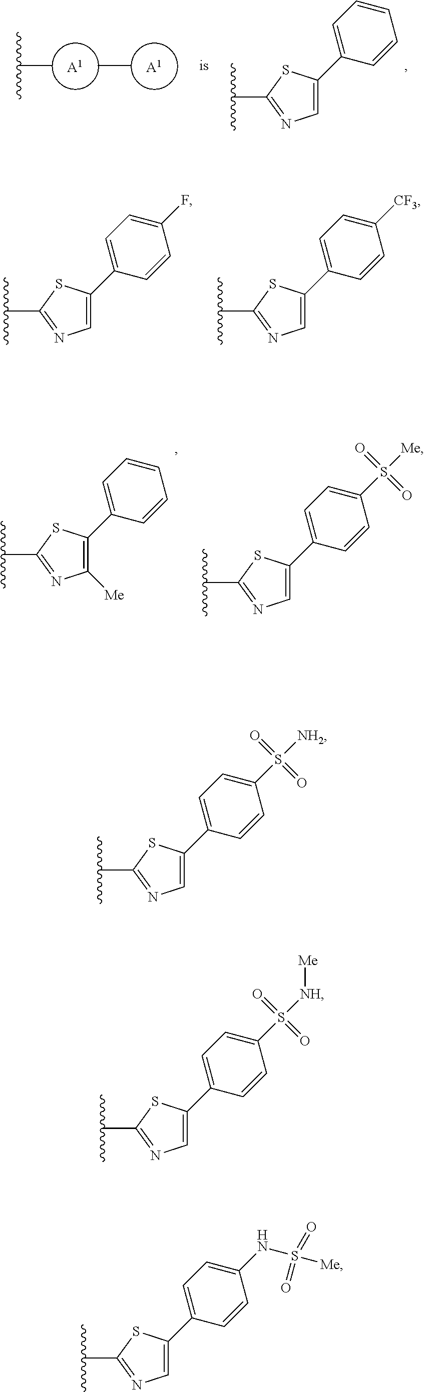

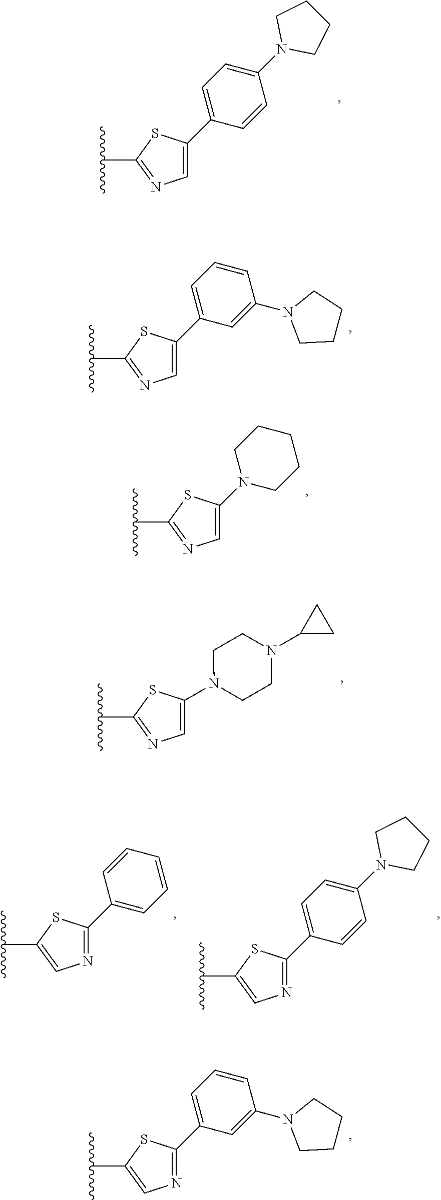

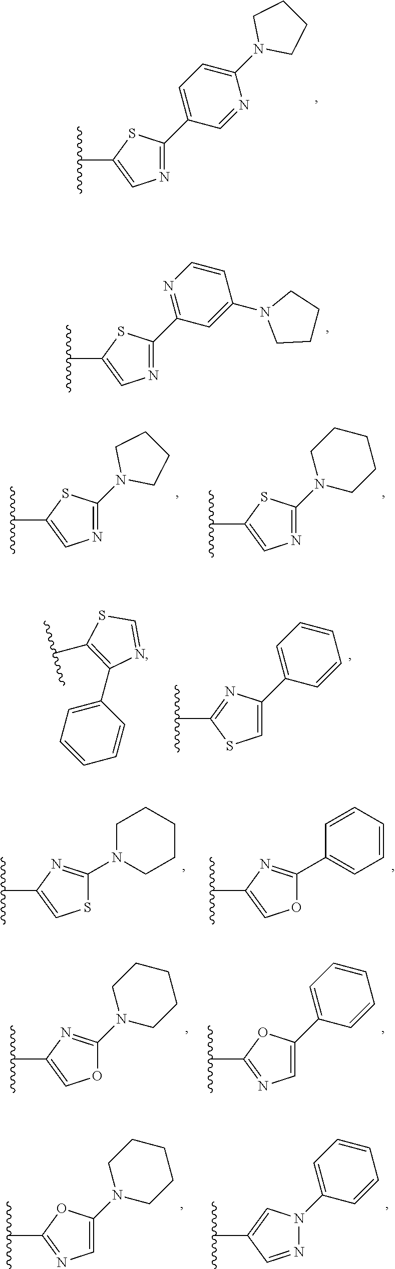

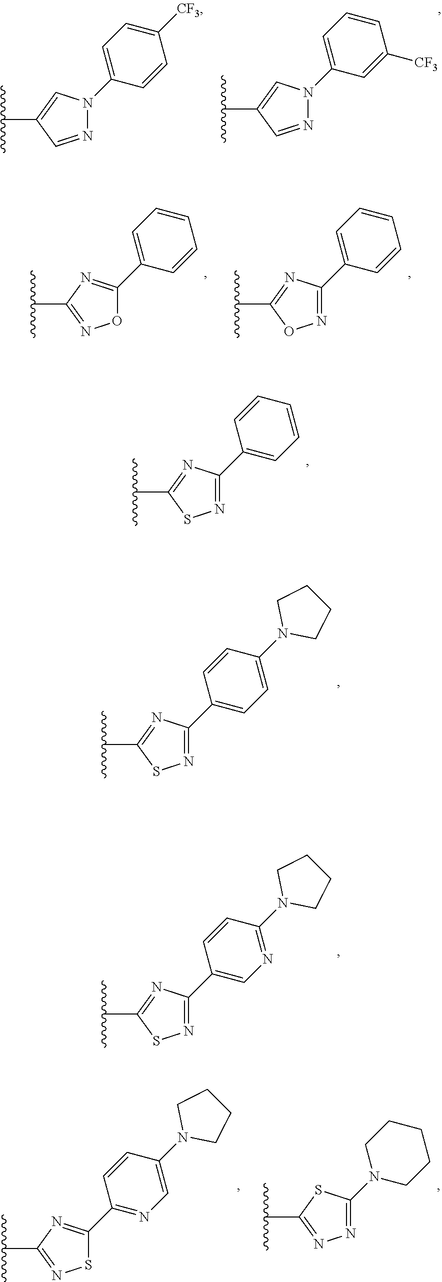

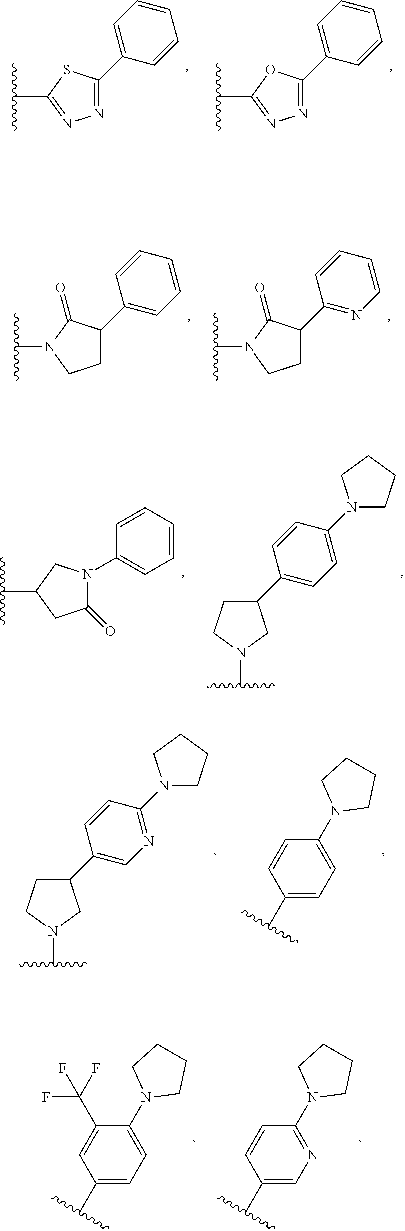

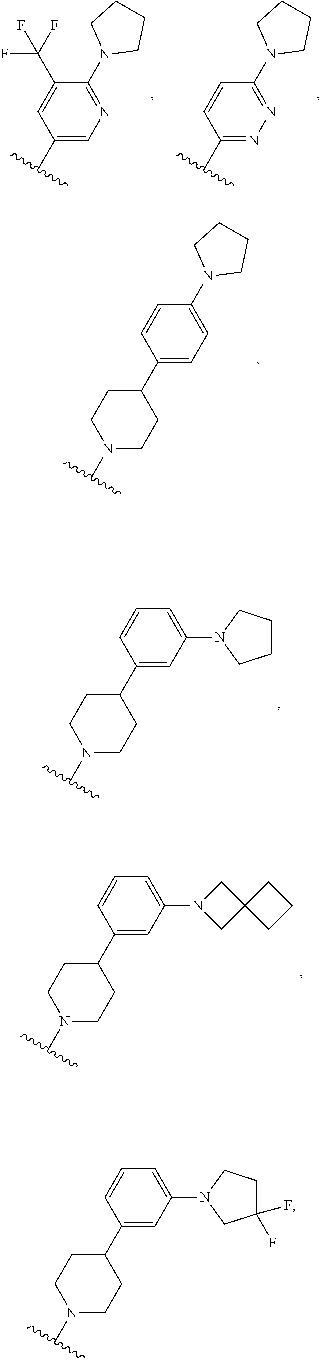 [SMILES: *c1cnc(N2CCCC2)c(C(F)(F)F)c1 ; *c1ccc(N2CCCC2)nn1 ; *N1CCC(c2ccc(N3CCCC3)cc2)CC1 ; *N1CCC(c2cccc(N3CCCC3)c2)CC1 ; *N1CCC(c2cccc(N3CC4(CCC4)C3)c2)CC1 ; *N1CCC(c2cccc(N3CCC(F)(F)C3)c2)CC1]

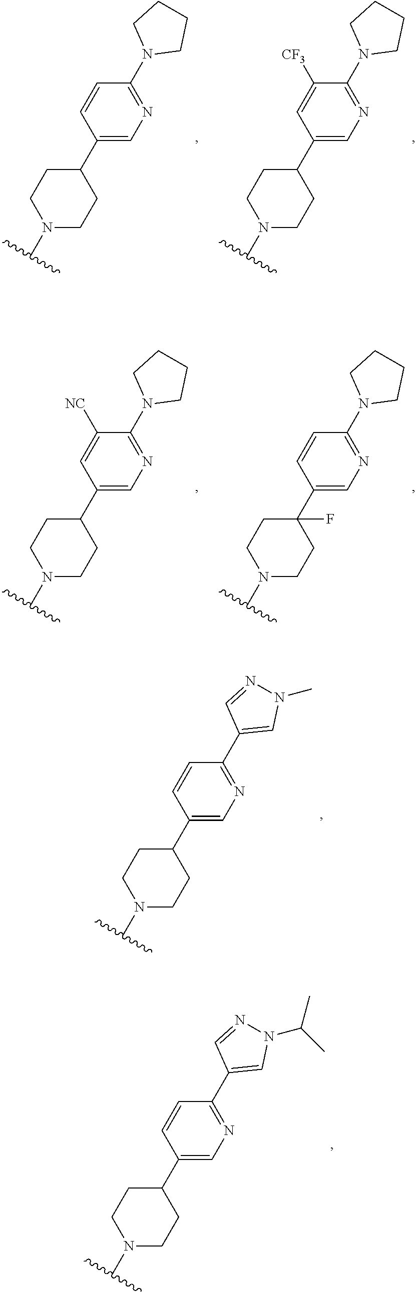 [SMILES: *N1CCC(c2ccc(N3CCCC3)nc2)CC1 ; *N1CCC(c2cnc(N3CCCC3)c(C(F)(F)F)c2)CC1 ; *N1CCC(c2cnc(N3CCCC3)c(C#N)c2)CC1 ; *N1CCC(F)(c2ccc(N3CCCC3)nc2)CC1 ; *N1CCC(c2ccc(-c3cnn(C)c3)nc2)CC1 ; *N1CCC(c2ccc(-c3cnn(C(C)C)c3)nc2)CC1]

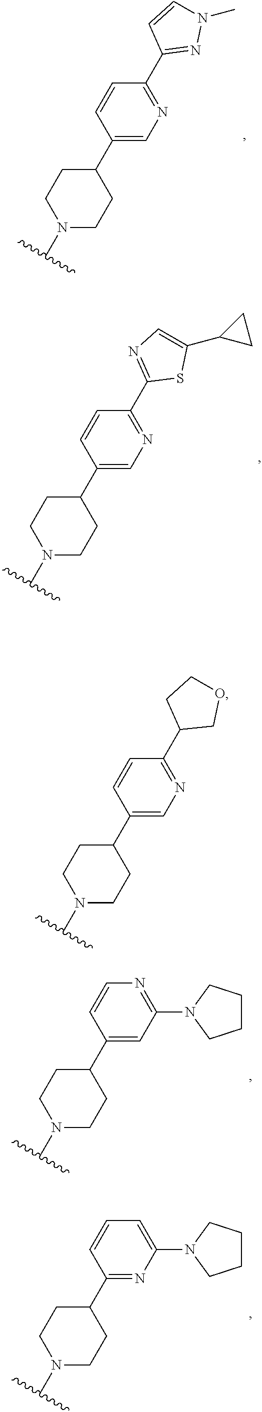 [SMILES: *N1CCC(c2ccc(-c3ccn(C)n3)nc2)CC1 ; *N1CCC(c2ccc(-c3ncc(C4CC4)s3)nc2)CC1 ; *N1CCC(c2ccc(C3CCOC3)nc2)CC1 ; *N1CCC(c2ccnc(N3CCCC3)c2)CC1 ; *N1CCC(c2cccc(N3CCCC3)n2)CC1]

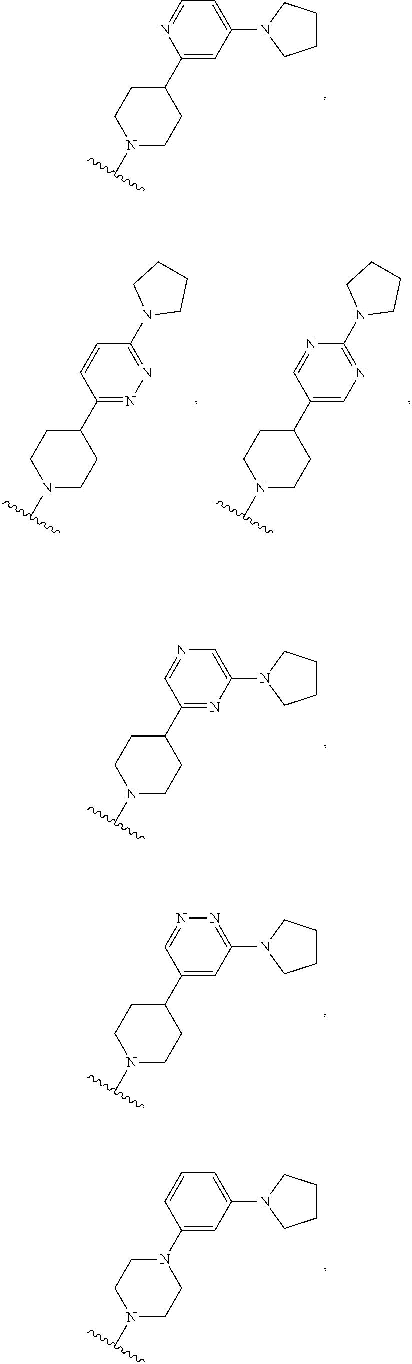 [SMILES: *N1CCC(c2cc(N3CCCC3)ccn2)CC1 ; *N1CCC(c2ccc(N3CCCC3)nn2)CC1 ; *N1CCC(c2cnc(N3CCCC3)nc2)CC1 ; *N1CCC(c2cncc(N3CCCC3)n2)CC1 ; *N1CCC(c2cnnc(N3CCCC3)c2)CC1 ; *N1CCN(c2cccc(N3CCCC3)c2)CC1]

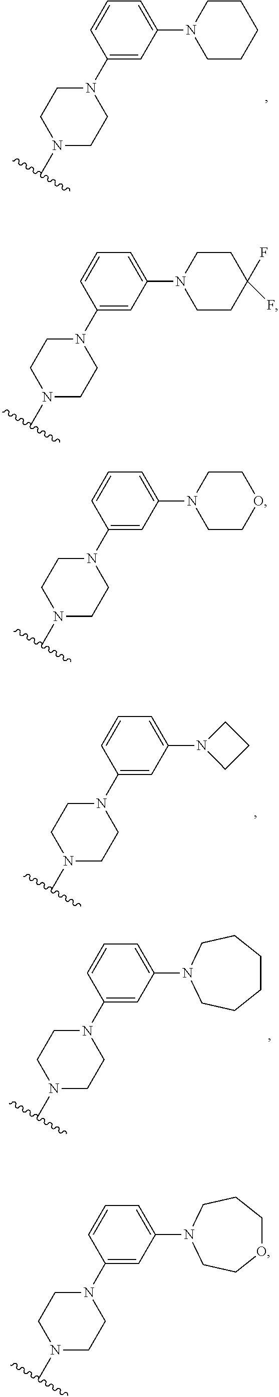 [SMILES: *N1CCN(c2cccc(N3CCCCC3)c2)CC1 ; *N1CCN(c2cccc(N3CCC(F)(F)CC3)c2)CC1 ; *N1CCN(c2cccc(N3CCOCC3)c2)CC1 ; *N1CCN(c2cccc(N3CCC3)c2)CC1 ; *N1CCN(c2cccc(N3CCCCCC3)c2)CC1 ; *N1CCN(c2cccc(N3CCCOCC3)c2)CC1]

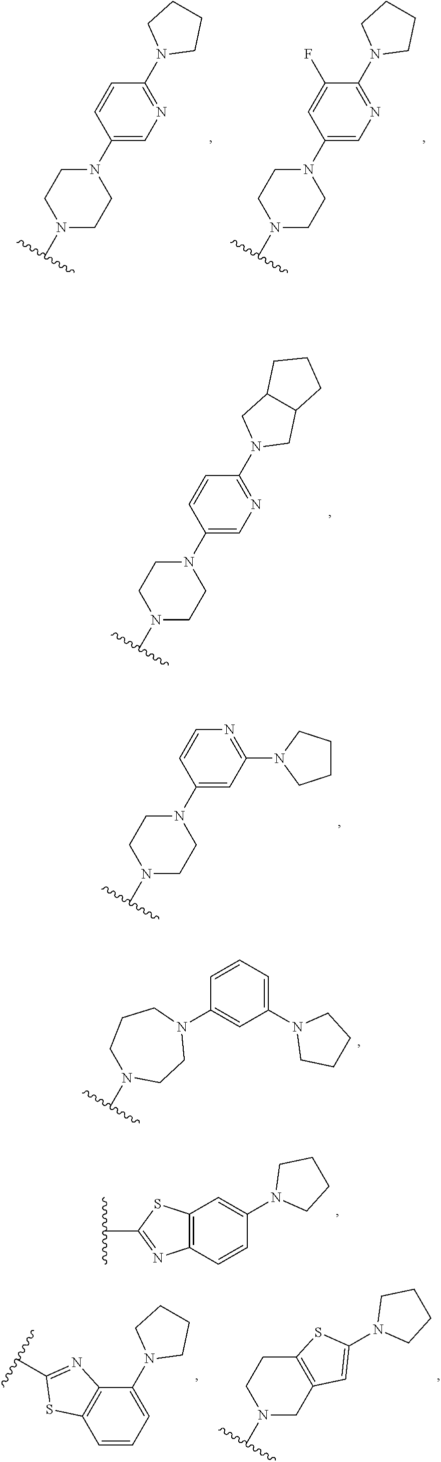

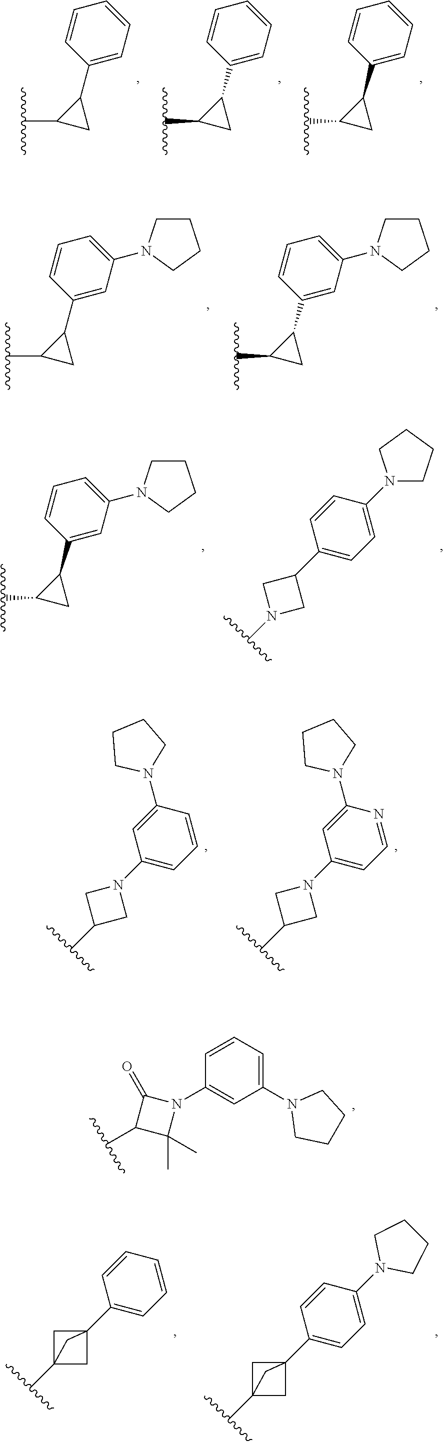

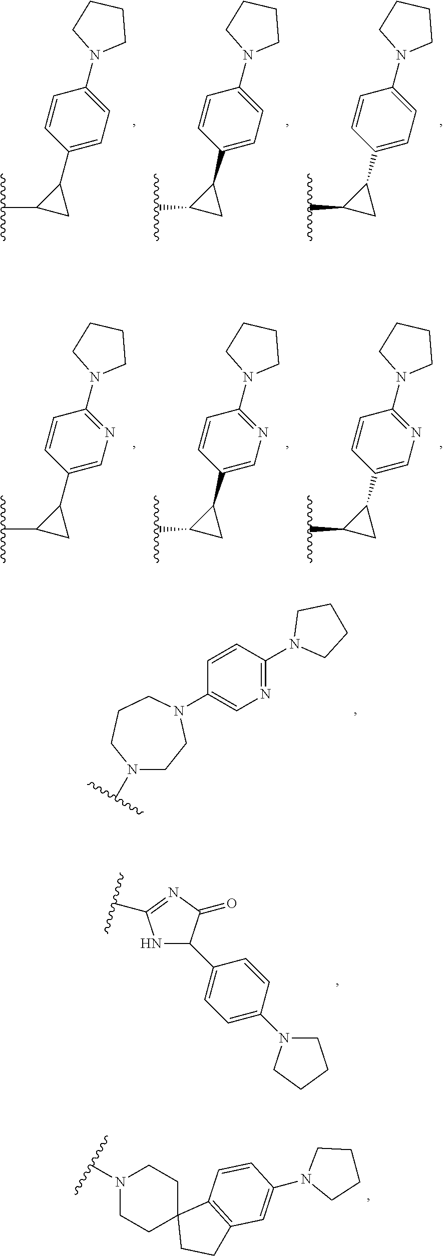

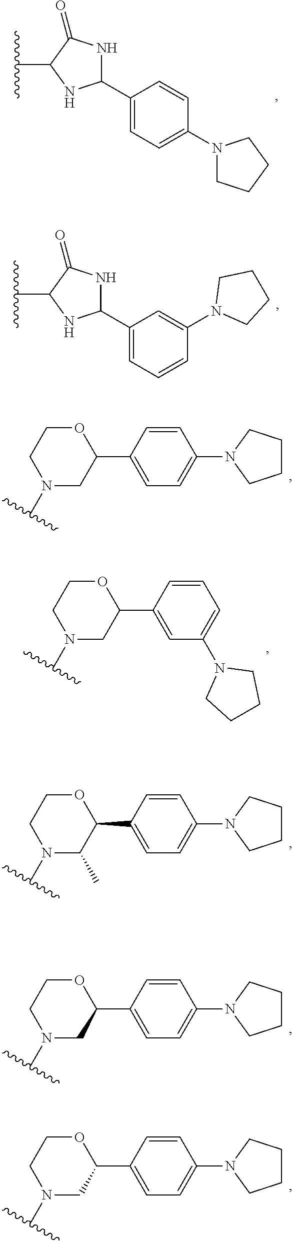

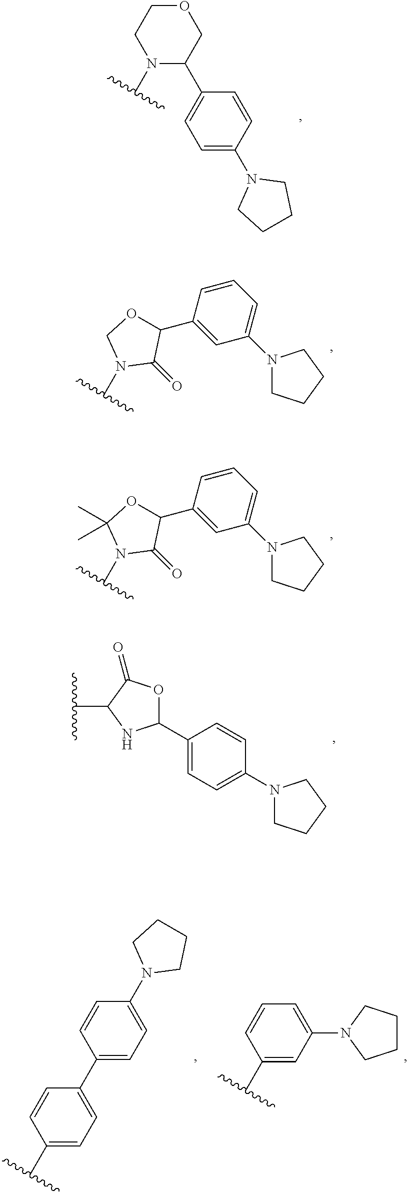 [SMILES: *N1CCOCC1c1ccc(N2CCCC2)cc1 ; *N1COC(c2cccc(N3CCCC3)c2)C1=O ; *N1C(=O)C(c2cccc(N3CCCC3)c2)OC1(C)C ; *C1NC(c2ccc(N3CCCC3)cc2)OC1=O ; *c1ccc(-c2ccc(N3CCCC3)cc2)cc1 ; *c1cccc(N2CCCC2)c1]

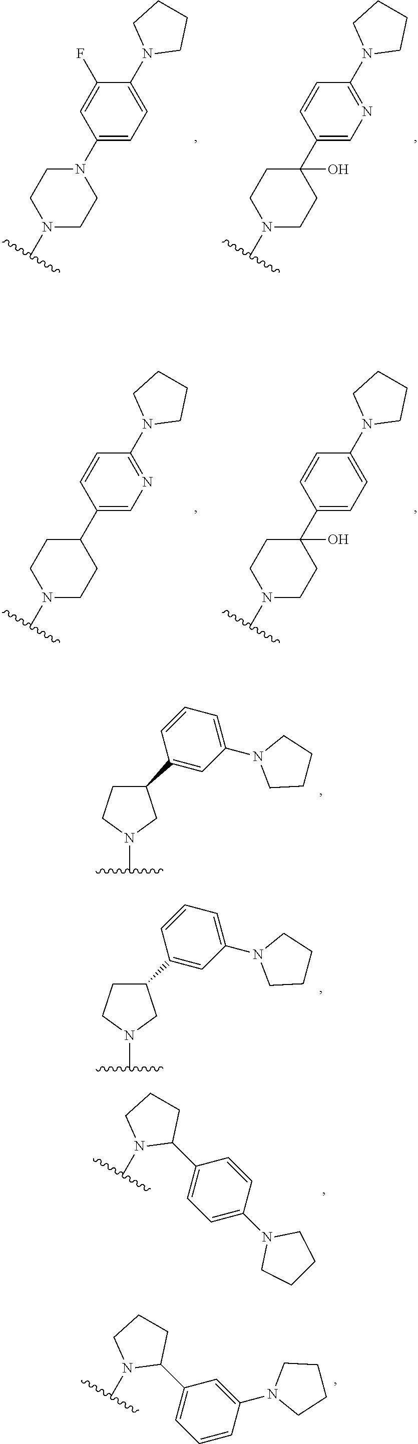

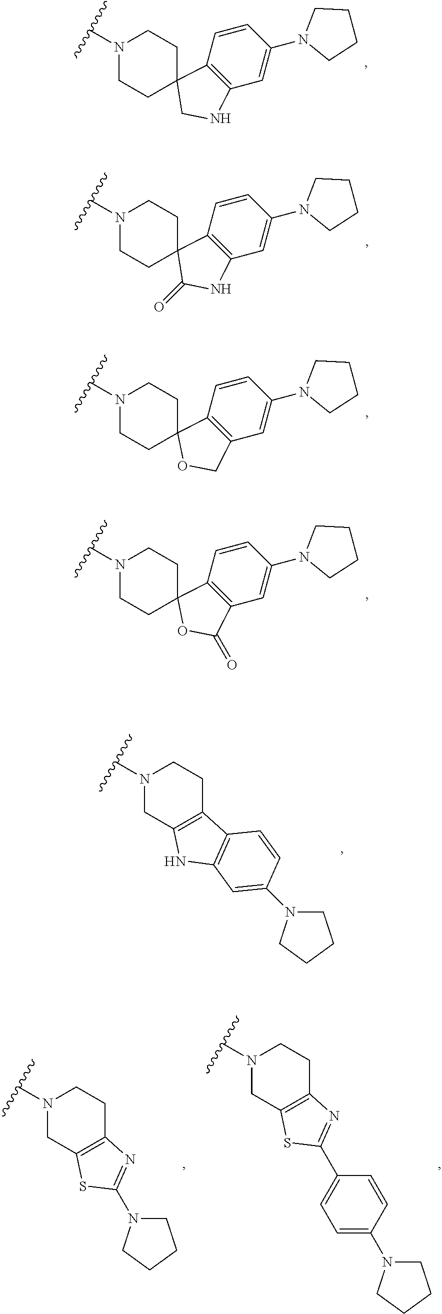

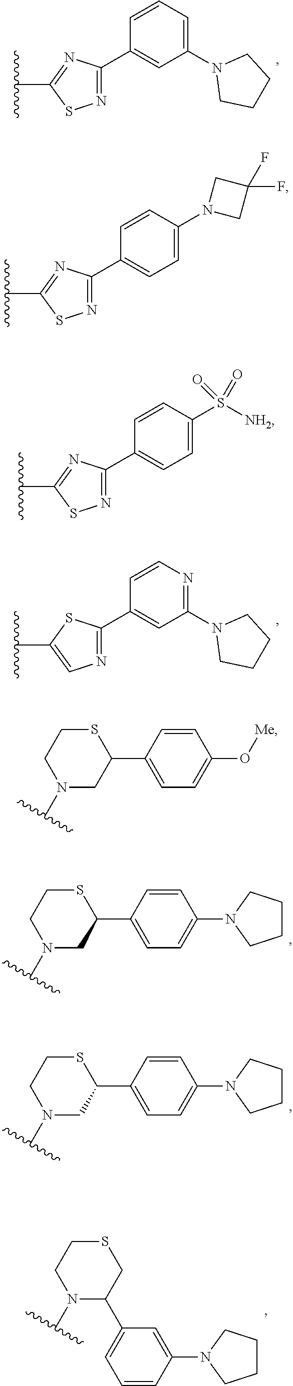

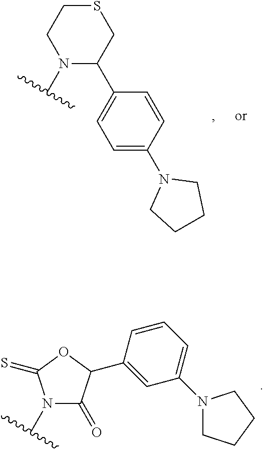 [SMILES: *N1CCSCC1c1ccc(N2CCCC2)cc1 ; *N1C(=O)C(c2cccc(N3CCCC3)c2)OC1=S]

In some variations,

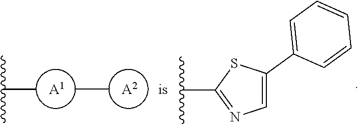

In some variations,

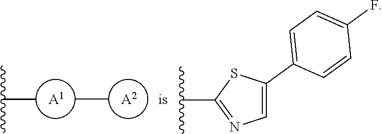

In some variations,

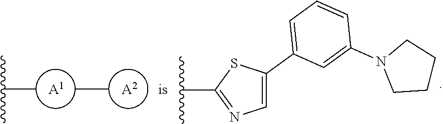

In some embodiments of Formula (I), or a tautomer thereof, or a pharmaceutically acceptable salt of any of the foregoing, the

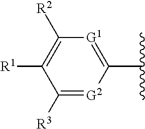

portion of the compound is

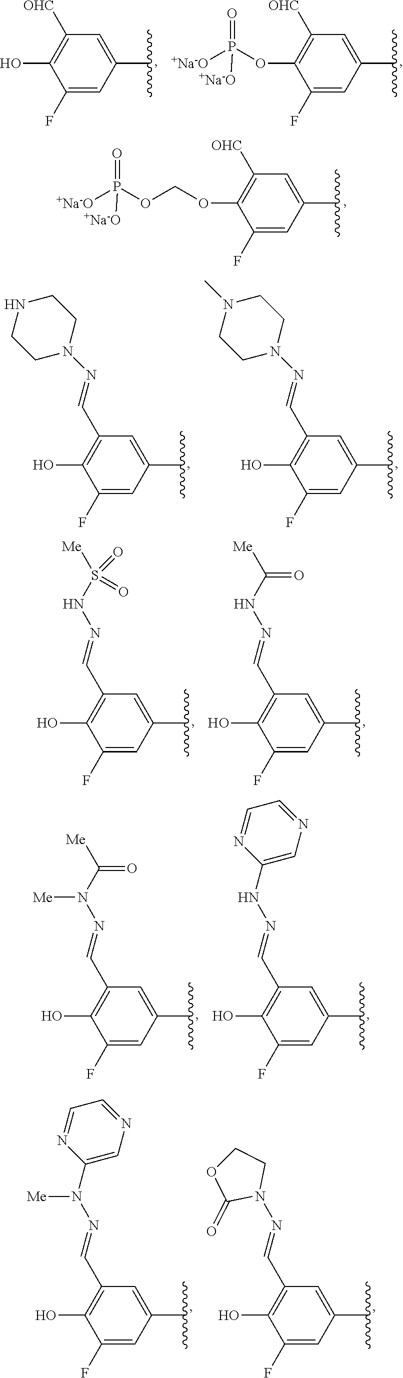

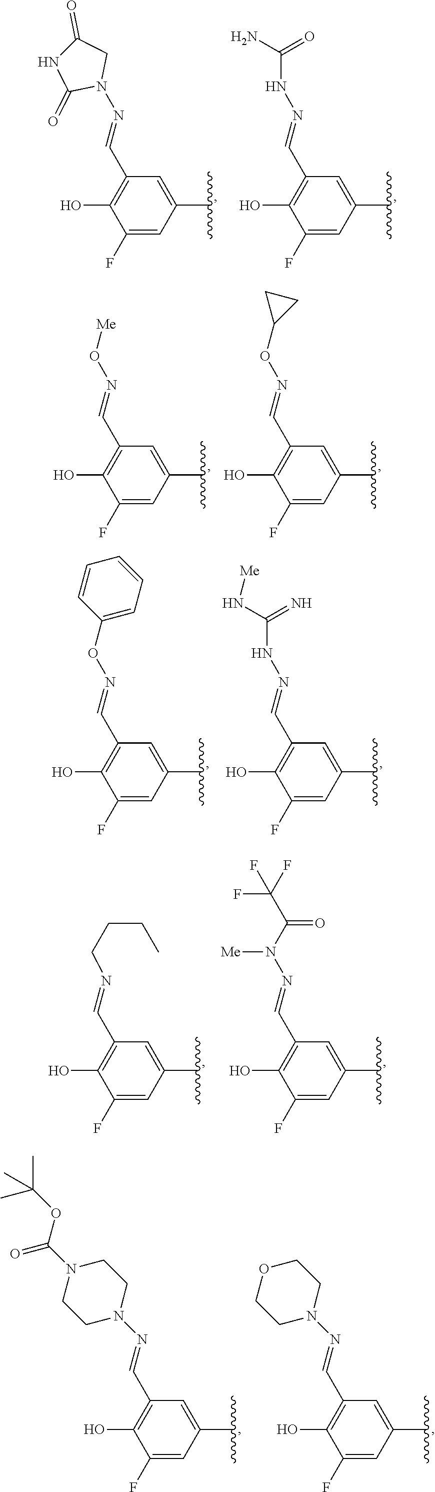

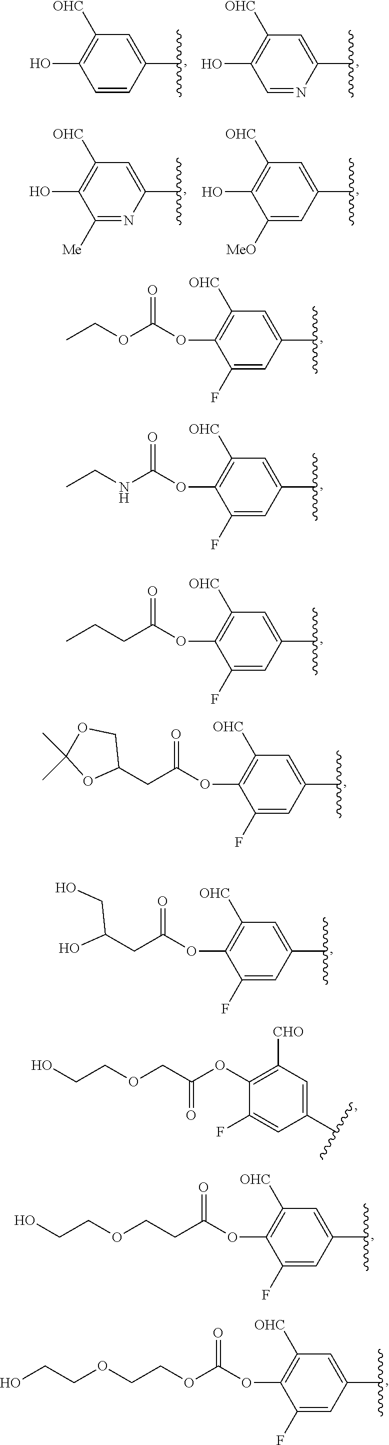

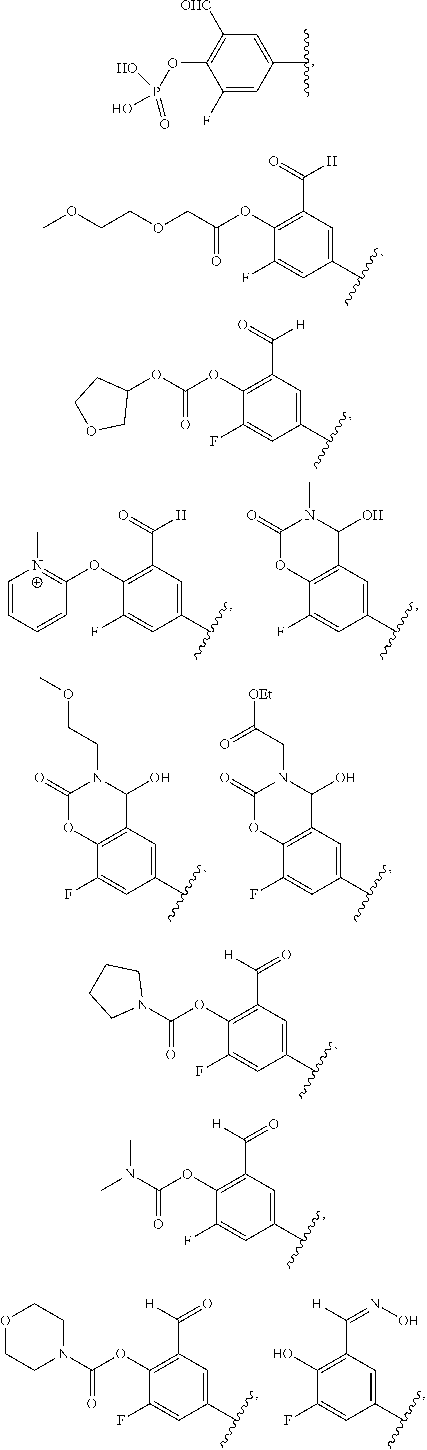

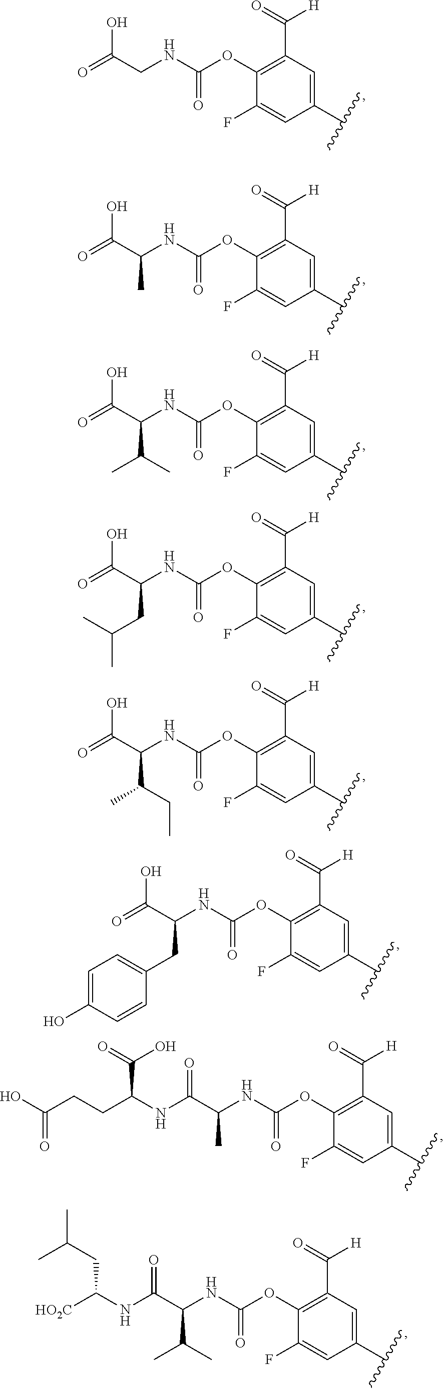

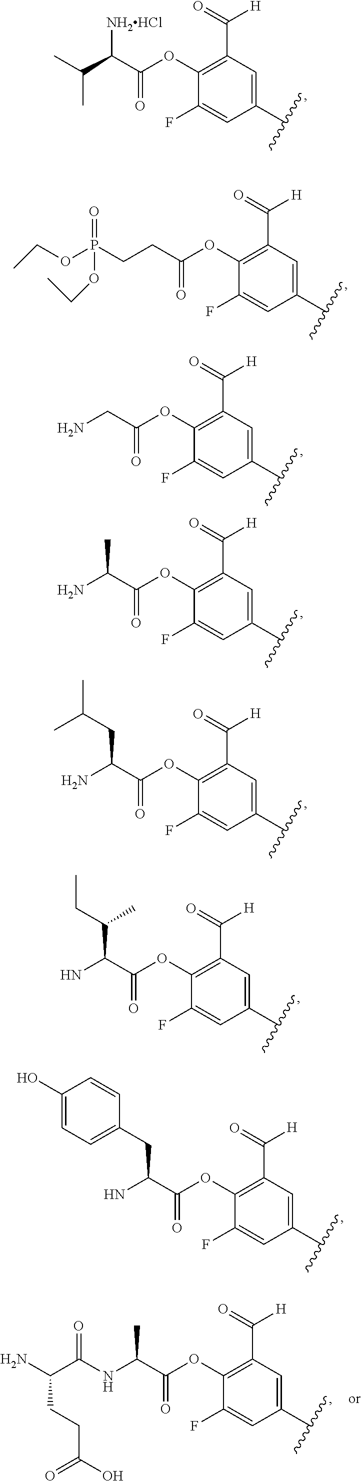

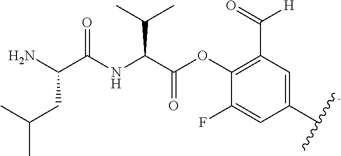 [SMILES: *c1cc(F)c(OC(=O)[C@@H](NC(=O)[C@@H](N)CC(C)C)C(C)C)c(C=O)c1]

In some variations, the

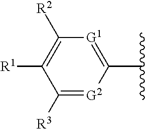

portion of the compound is

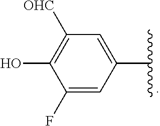 [SMILES: *c1cc(F)c(O)c(C=O)c1]

In some embodiments, the compound of Formula (I) is a compound of Formula (Ia-1):

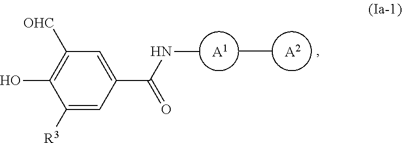

or a tautomer thereof, or a pharmaceutically acceptable salt of any of the foregoing, wherein R³, A¹, and A² are as defined for Formula (I).

In some embodiments, the compound of Formula (I) is a compound of Formula (Ia-2):

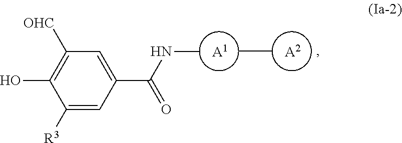

or a tautomer thereof, or a pharmaceutically acceptable salt of any of the foregoing, wherein R³ and A² are as defined for Formula (I); and A¹ is a 5-membered heteroaryl.

In some embodiments, the compound of Formula (I) is a compound of Formula (Ia-3):

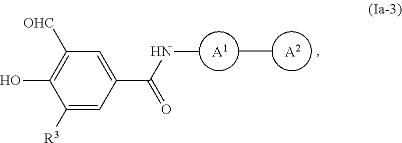

or a tautomer thereof, or a pharmaceutically acceptable salt of any of the foregoing, wherein A² is as defined for Formula (I); A¹ is a 5-membered heteroaryl; and R³ is halo.

In some embodiments, the compound of Formula (I) is a compound of Formula (Ib-1):

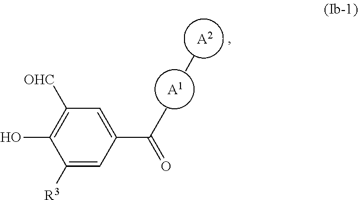

or a tautomer thereof, or a pharmaceutically acceptable salt of any of the foregoing, wherein R³, A¹, and A² are as defined for Formula (I).

In some embodiments, the compound of Formula (I) is a compound of Formula (Ib-2):

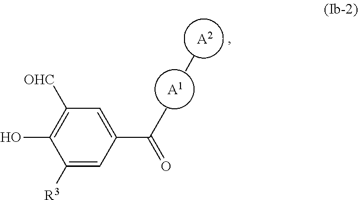

or a tautomer thereof, or a pharmaceutically acceptable salt of any of the foregoing, wherein A¹ and A² are as defined for Formula (I); and R³ is halo.

In some embodiments, the compound of Formula (I) is a compound of Formula (Ib-3):

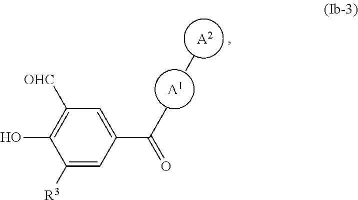

or a tautomer thereof, or a pharmaceutically acceptable salt of any of the foregoing, wherein A¹ is optionally substituted heterocyclyl; A² is phenyl substituted with heterocyclyl; and R³ is halo. In some variations, A¹ is piperidinyl. In some variations, A² is phenyl substituted with pyrrolidinyl. In some variations, R³ is fluoro.

In some embodiments, the compound of Formula (I) is a compound of Formula (Ic-1):

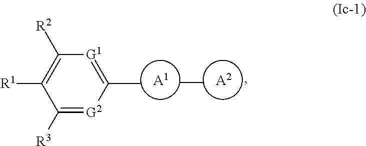

or a tautomer thereof, or a pharmaceutically acceptable salt of any of the foregoing, wherein R², R³, G¹, G², A¹, and A² are as defined for Formula (I); and R¹ is —OPO₃H₂, —OCH₂OPO₃H₂, —OC(O)R^{1A1}, —OC(O)OR^{1A1}, or —OC(O)NR^{1A1}R^{1A2}, wherein R^{1A1} and R^{1A2} are each independently hydrogen, optionally substituted alkyl, optionally substituted alkenyl, optionally substituted alkynyl, optionally substituted cycloalkyl, optionally substituted cycloalkenyl, optionally substituted aryl, optionally substituted heterocyclyl, optionally substituted heteroaryl, or —O₀₋₁(CH₂)ₘO(CH₂)ₙOH, and wherein m and n are each independently 1 or 2.

In some embodiments, the compound of Formula (I) is a compound of Formula (Ic-2):

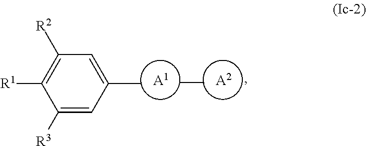

or a tautomer thereof, or a pharmaceutically acceptable salt of any of the foregoing, wherein R¹ is —OC(O)R^{1A1} or —OC(O)NR^{1A1}R^{1A2}; R² is —CHO; R³ is halo; A¹ is optionally substituted 5-membered heteroaryl; A² is phenyl substituted with heterocyclyl; and R^{1A1} and R^{1A2} are as defined for Formula (I). In some variations, A¹ is 1,2,4-thiadiazolyl. In some variations, A² is phenyl substituted with pyrrolidinyl. In some variations, R³ is fluoro.

In some embodiments, the compound of Formula (I) is a compound of Formula (Id-1):

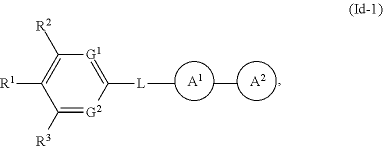

or a tautomer thereof, or a pharmaceutically acceptable salt of any of the foregoing, wherein R¹ is R^{1A}; R² is R^{2A}; and R^{1A}, R^{2A}, R³, G¹, G², L, A¹, and A² are as defined for Formula (I).

In some embodiments, the compound of Formula (I) is a compound of Formula (Id-1):

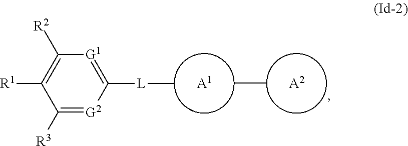

or a tautomer thereof, or a pharmaceutically acceptable salt of any of the foregoing, wherein R¹ is R^{2A}; R² is R^{1A}; and R^{1A}, R^{2A}, R³, G¹, G², L, A¹, and A² are as defined for Formula (I).

In some embodiments, when any particular group is substituted, the indicated group is substituted by one or more substituents independently selected from the group consisting of oxo, C₁-C₆ alkyl, C₂-C₆ alkenyl, C₂-C₆ alkynyl, halogen, —CN, —OR^{A1}, —SR^{A1}, —NR^{A2}R^{A3}, —NO₂, —C═NH(OR^{A1}), —C(O)R^{A1}, —OC(O)R^{A1}, —C(O)OR^{A1}, —C(O)NR^{A2}R^{A3}, —OC(O)NR^{A2}R^{A3}, —NR^{A1}C(O)R^{A2}, —NR^{A1}C(O)OR^{A2}, —NR^{A1}C(O)NR^{A2}R^{A3}, —S(O)R^{A1}, —S(O)₂R^{A1}, —NR^{A1}S(O)R^{A2}, —C(O)NR^{A1}S(O)R^{A2}, —NR^{A1}S(O)₂R^{A2}, —C(O)NR^{A1}S(O)₂R^{A2}, —S(O)NR^{A2}R^{A3}, —S(O)₂NR^{A2}R^{A3}, —P(O)(OR^{A2})(OR^{A3}), C₃-C₈ cycloalkyl, 3- to 12-membered heterocyclyl, 5- to 10-membered heteroaryl, C₆-C₁₄ aryl, —(C₁-C₃ alkylene)CN, —(C₁-C₃ alkylene)OR^{A1}, —(C₁-C₃ alkylene)SR^{A1}, —(C₁-C₃ alkylene)NR^{A2}R^{A3}, —(C₁-C₃ alkylene)CF₃, —(C₁-C₃ alkylene)NO₂, —C═NH(OR^{A1}), —(C₁-C₃ alkylene)C(O)R^{A1}, —(C₁-C₃ alkylene)OC(O)R^{A1}, —(C₁-C₃ alkylene)C(O)OR^{A1}, —(C₁-C₃ alkylene)C(O)NR^{A2}R^{A3}, —(C₁-C₃ alkylene)OC(O)NR^{A2}R^{A}, —(C₁-C₃ alkylene)NR^{A1}C(O)R^{A2}, —(C₁-C₃ alkylene)NR^{A1}C(O)OR^{A2}, —(C₁-C₃ alkylene)NR^{A1}C(O)NR^{A2}R^{A3}, —(C₁-C₃ alkylene)S(O)R^{A1}, —(C₁-C₃ alkylene)S(O)₂R^{A1}, —(C₁-C₃ alkylene)NR^{A1}S(O)R^{A2}, —C(O)(C₁-C₃ alkylene)NR^{A1}S(O)R^{A2}, —(C₁-C₃ alkylene)NR^{A1}S(O)₂R^{A2}, —(C₁-C₃ alkylene)C(O)NR^{A1}S(O)₂R^{A2}, —(C₁-C₃ alkylene)S(O)NR^{A2}R^{A3}, —(C₁-C₃ alkylene)S(O)₂NR^{A2}R^{A3}, —(C₁-C₃ alkylene)P(O)(OR^{A2})(OR^{A3}), —(C₁-C₃ alkylene)(C₃-C₈ cycloalkyl), —(C₁-C₃ alkylene)(3- to 12-membered heterocyclyl), —(C₁-C₃ alkylene)(5- to 10-membered heteroaryl) and —(C₁-C₃ alkylene)(C₆-C₁₄ aryl), wherein the one or more substituents are each independently unsubstituted or substituted with one or more further substituents independently selected from the group consisting of halogen, oxo, —OR^{A4}, —NR^{A4}R^{A5}, —C(O)R^{A4}, —CN, —S(O)R^{A4}, —S(O)₂R^{A4}, —P(O)(OR^{A4})(OR^{A5}), —(C₁-C₃ alkylene)OR^{A4}, —(C₁-C₃ alkylene)NR^{A4}R^{A5}, —(C₁-C₃ alkylene)C(O)R^{A4}, —(C₁-C₃ alkylene)S(O)R^{A4}, —(C₁-C₃ alkylene)S(O)₂R^{A4}, —(C₁-C₃ alkylene)P(O)(OR^{A4})(OR^{A5}), C₃-C₈ cycloalkyl, C₁-C₆ alkyl, and C₁-C₆ alkyl substituted by oxo, —OH or halogen; wherein each R^{A1} is independently hydrogen, C₁-C₆ alkyl, C₂-C₆ alkenyl, C₂-C₆ alkynyl, C₃-C₈ cycloalkyl, C₆-C₁₄ aryl, 5- to 6-membered heteroaryl or 3- to 6-membered heterocyclyl, wherein the C₁-C₆ alkyl, C₂-C₆ alkenyl, C₂-C₆ alkynyl, C₃-C₈ cycloalkyl, C₆-C₁₄ aryl, 5- to 6-membered heteroaryl and 3- to 6-membered heterocyclyl are independently unsubstituted or substituted by halogen, oxo, —CN, —OR^{A6}, —NR^{A6}R^{A7}, —P(O)(OR^{A6})(OR^{A6}), phenyl, phenyl substituted by halogen, C₁-C₆ alkyl, or C₁-C₆ alkyl substituted by halogen, —OH or oxo; R^{A2} and R^{A3} are each independently hydrogen, C₁-C₆ alkyl, C₂-C₆ alkenyl, C₂-C₆ alkynyl, C₃-C₈ cycloalkyl, C₆-C₁₄ aryl, 5- to 6-membered heteroaryl or 3- to 6-membered heterocyclyl, wherein the C₁-C₆ alkyl, C₂-C₆ alkenyl, C₂-C₆ alkynyl, C₃-C₈ cycloalkyl, C₆-C₁₄ aryl, 5- to 6-membered heteroaryl and 3- to 6-membered heterocyclyl are each independently unsubstituted or substituted by halogen, oxo, —CN, —OR^{A6}, —NR^{A6}R^{A7}, C₁-C₆ alkyl, or C₁-C₆ alkyl substituted by halogen, —OH or oxo; and R^{A4}, R^{A5}, R^{A6} and R^{A7} are each independently hydrogen, C₁-C₆ alkyl, C₂-C₆ alkenyl, C₂-C₆ alkynyl, C₁-C₆ alkyl substituted by one or more halogen, C₂-C₆ alkenyl substituted by one or more halogen, or C₂-C₆ alkynyl substituted by one or more halogen.

In some embodiments, provided herein are compounds and salts thereof described in Table 1.

TABLE 1

| Compound | | |
|---|---|---|
| No. | Chemical Structure | Chemical Name |
| 1 | 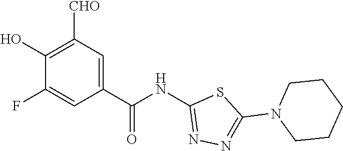 [SMILES: O=Cc1cc(C(=O)Nc2nnc(N3CCCCC3)s2)cc(F)c1O] | 3-fluoro-5-formyl-4-hydroxy- N-(5-(piperidin-1-yl)-1,3,4- thiadiazol-2-yl)benzamide |
| 2 | 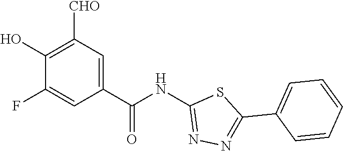 [SMILES: O=Cc1cc(C(=O)Nc2nnc(-c3ccccc3)s2)cc(F)c1O] | 3-fluoro-5-formyl-4-hydroxy- N-(5-phenyl-1,3,4-thiadiazol- 2-yl)benzamide |
| 3 | 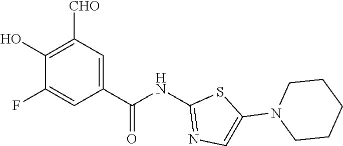 [SMILES: O=Cc1cc(C(=O)Nc2ncc(N3CCCCC3)s2)cc(F)c1O] | 3-fluoro-5-formyl-4-hydroxy- N-(5-(piperidin-1-yl)thiazol-2- yl)benzamide |
| 4 | 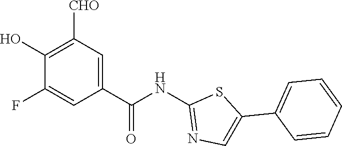 [SMILES: O=Cc1cc(C(=O)Nc2ncc(-c3ccccc3)s2)cc(F)c1O] | 3-fluoro-5-formyl-4-hydroxy- N-(5-phenylthiazol-2- yl)benzamide |
| 5 | 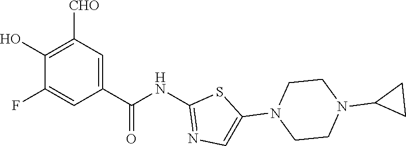 [SMILES: O=Cc1cc(C(=O)Nc2ncc(N3CCN(C4CC4)CC3)s2)cc(F)c1O] | N-(5-(4-cyclopropylpiperazin- 1-yl)thiazol-2-yl)-3-fluoro-5- formyl-4-hydroxybenzamide |
| 6 | 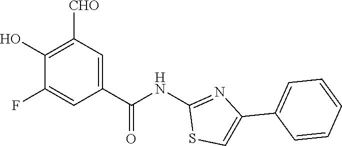 [SMILES: O=Cc1cc(C(=O)Nc2nc(-c3ccccc3)cs2)cc(F)c1O] | 3-fluoro-5-formyl-4-hydroxy- N-(4-phenylthiazol-2- yl)benzamide |
| 7 | 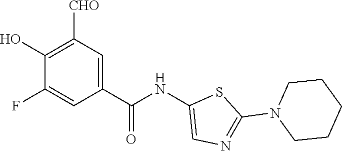 [SMILES: O=Cc1cc(C(=O)Nc2cnc(N3CCCCC3)s2)cc(F)c1O] | 3-fluoro-5-formyl-4-hydroxy- N-(2-(piperidin-1-yl)thiazol-5- yl)benzamide |
| 8 | 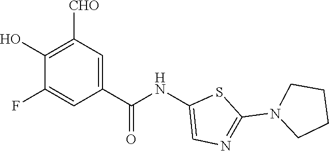 [SMILES: O=Cc1cc(C(=O)Nc2cnc(N3CCCC3)s2)cc(F)c1O] | 3-fluoro-5-formyl-4-hydroxy- N-(2-(pyrrolidin-1-yl)thiazol- 5-yl)benzamide |
| 9 | 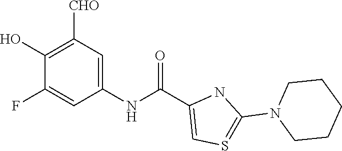 | N-(3-fluoro-5-formyl-4- hydroxyphenyl)-2-(piperidin- 1-yl)thiazole-4-carboxamide |
| 10 | 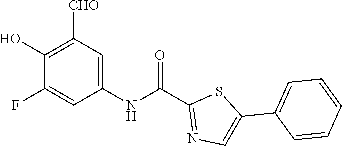 [SMILES: O=Cc1cc(NC(=O)c2ncc(-c3ccccc3)s2)cc(F)c1O] | N-(3-fluoro-5-formyl-4- hydroxyphenyl)-5- phenylthiazole-2-carboxamide |
| 11 | 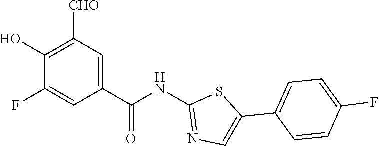 [SMILES: O=Cc1cc(C(=O)Nc2ncc(-c3ccc(F)cc3)s2)cc(F)c1O] | 3-fluoro-N-(5-(4- fluorophenyl)thiazol-2-yl)-5- formyl-4-hydroxybenzamide |
| 12 | 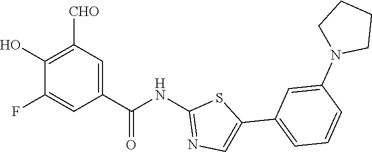 [SMILES: O=Cc1cc(C(=O)Nc2ncc(-c3cccc(N4CCCC4)c3)s2)cc(F)c1O] | 3-fluoro-5-formyl-4-hydroxy- N-(5-(3-(pyrrolidin-1- yl)phenyl)thiazol-2- yl)benzamide |
| 13 | 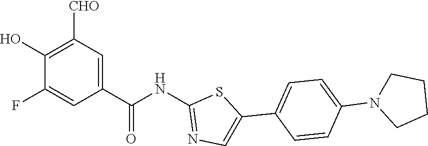 [SMILES: O=Cc1cc(C(=O)Nc2ncc(-c3ccc(N4CCCC4)cc3)s2)cc(F)c1O] | 3-fluoro-5-formyl-4-hydroxy- N-(5-(4-(pyrrolidin-1- yl)phenyl)thiazol-2- yl)benzamide |
| 14 | 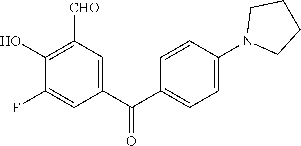 [SMILES: O=Cc1cc(C(=O)c2ccc(N3CCCC3)cc2)cc(F)c1O] | 3-fluoro-2-hydroxy-5-(4- (pyrrolidin-1- yl)benzoyl)benzaldehyde |
| 15 | 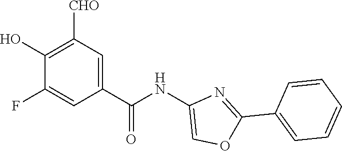 [SMILES: O=Cc1cc(C(=O)Nc2coc(-c3ccccc3)n2)cc(F)c1O] | 3-fluoro-5-formyl-4-hydroxy- N-(2-phenyloxazol-4- yl)benzamide |
| 16 | 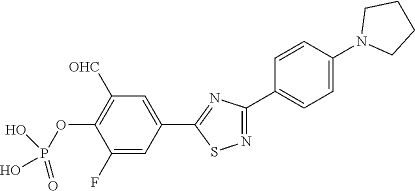 [SMILES: O=Cc1cc(-c2nc(-c3ccc(N4CCCC4)cc3)ns2)cc(F)c1OP(=O)(O)O] | 2-fluoro-6-formyl-4-(3-(4- (pyrrolidin-1-yl)phenyl)-1,2,4- thiadiazol-5-yl)phenyl dihydrogen phosphate |
| 17 | 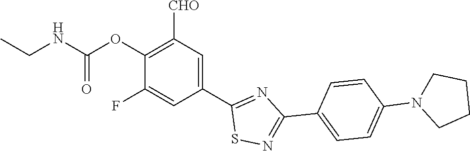 [SMILES: CCNC(=O)Oc1c(F)cc(-c2nc(-c3ccc(N4CCCC4)cc3)ns2)cc1C=O] | 2-fluoro-6-formyl-4-(3-(4- (pyrrolidin-1-yl)phenyl)-1,2,4- thiadiazol-5-yl)phenyl ethylcarbamate |
| 18 | 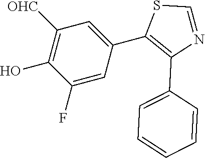 [SMILES: O=Cc1cc(-c2scnc2-c2ccccc2)cc(F)c1O] | 3-fluoro-2-hydroxy-5-(4- phenylthiazol-5- yl)benzaldehyde |
| 19 | 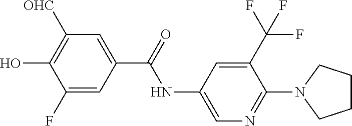 [SMILES: O=Cc1cc(C(=O)Nc2cnc(N3CCCC3)c(C(F)(F)F)c2)cc(F)c1O] | 3-fluoro-5-formyl-4-hydroxy- N-(6-(pyrrolidin-1-yl)-5- (trifluoromethyl)pyridin-3- yl)benzamide |
| 20 | 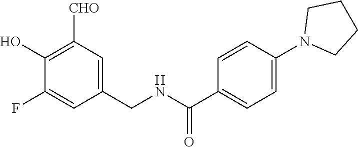 [SMILES: O=Cc1cc(CNC(=O)c2ccc(N3CCCC3)cc2)cc(F)c1O] | N-(3-fluoro-5-formyl-4- hydroxybenzyl)-4-(pyrrolidin- 1-yl)benzamide |
| 21 | 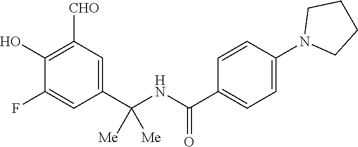 [SMILES: CC(C)(NC(=O)c1ccc(N2CCCC2)cc1)c1cc(F)c(O)c(C=O)c1] | N-(2-(3-fluoro-5-formyl-4- hydroxyphenyl)propan-2-yl)- 4-(pyrrolidin-1-yl)benzamide |
| 22 | 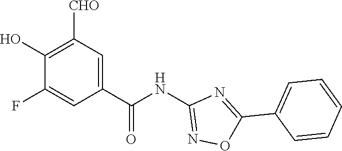 [SMILES: O=Cc1cc(C(=O)Nc2noc(-c3ccccc3)n2)cc(F)c1O] | 3-fluoro-5-formyl-4-hydroxy- N-(5-phenyl-1,2,4-oxadiazol- 3-yl)benzamide |
| 23 | 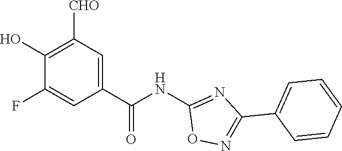 [SMILES: O=Cc1cc(C(=O)Nc2nc(-c3ccccc3)no2)cc(F)c1O] | 3-fluoro-5-formyl-4-hydroxy- N-(3-phenyl-1,2,4-oxadiazol- 5-yl)benzamide |
| 24 | 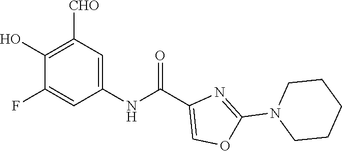 [SMILES: O=Cc1cc(NC(=O)c2coc(N3CCCCC3)n2)cc(F)c1O] | N-(3-fluoro-5-formyl-4- hydroxyphenyl)-2-(piperidin- 1-yl)oxazole-4-carboxamide |
| 25 | 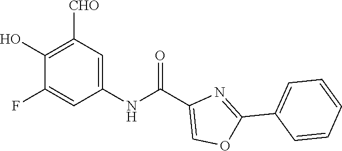 [SMILES: O=Cc1cc(NC(=O)c2coc(-c3ccccc3)n2)cc(F)c1O] | N-(3-fluoro-5-formyl-4- hydroxyphenyl)-2- phenyloxazole-4-carboxamide |
| 26 | 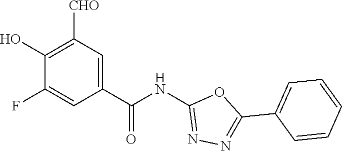 [SMILES: O=Cc1cc(C(=O)Nc2nnc(-c3ccccc3)o2)cc(F)c1O] | 3-fluoro-5-formyl-4-hydroxy- N-(5-phenyl-1,3,4-oxadiazol- 2-yl)benzamide |
| 27 | 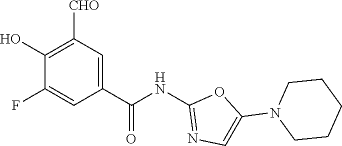 [SMILES: O=Cc1cc(C(=O)Nc2ncc(N3CCCCC3)o2)cc(F)c1O] | 3-fluoro-5-formyl-4-hydroxy- N-(5-(piperidin-1-yl)oxazol-2- yl)benzamide |
| 28 | 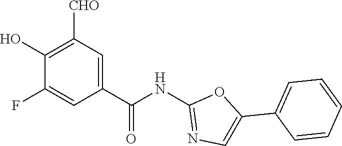 [SMILES: O=Cc1cc(C(=O)Nc2ncc(-c3ccccc3)o2)cc(F)c1O] | 3-fluoro-5-formyl-4-hydroxy- N-(5-phenyloxazol-2- yl)benzamide |
| 29 | 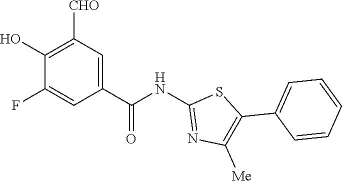 [SMILES: Cc1nc(NC(=O)c2cc(F)c(O)c(C=O)c2)sc1-c1ccccc1] | 3-fluoro-5-formyl-4-hydroxy- N-(4-methyl-5-phenylthiazol- 2-yl)benzamide |
| 30 | 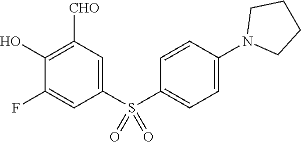 [SMILES: O=Cc1cc(S(=O)(=O)c2ccc(N3CCCC3)cc2)cc(F)c1O] | 3-fluoro-2-hydroxy-5-((4- (pyrrolidin-1- yl)phenyl)sulfonyl) benzaldehyde |
| 31 | 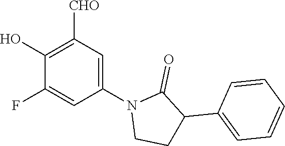 [SMILES: O=Cc1cc(N2CCC(c3ccccc3)C2=O)cc(F)c1O] | 3-fluoro-2-hydroxy-5-(2-oxo- 3-phenylpyrrolidin-1- yl)benzaldehyde |
| 32 | 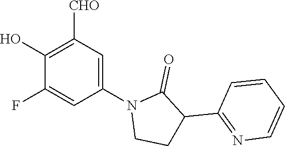 [SMILES: O=Cc1cc(N2CCC(c3ccccn3)C2=O)cc(F)c1O] | 3-fluoro-2-hydroxy-5-(2-oxo- 3-(pyridin-2-yl)pyrrolidin-1- yl)benzaldehyde |
| 33 | 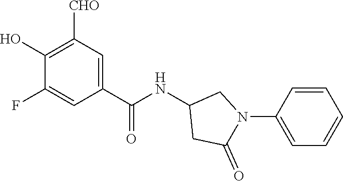 [SMILES: O=Cc1cc(C(=O)NC2CC(=O)N(c3ccccc3)C2)cc(F)c1O] | 3-fluoro-5-formyl-4-hydroxy- N-(5-oxo-1-phenylpyrrolidin- 3-yl)benzamide |
| 34 | 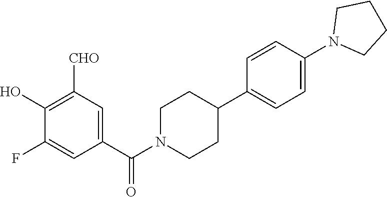 [SMILES: O=Cc1cc(C(=O)N2CCC(c3ccc(N4CCCC4)cc3)CC2)cc(F)c1O] | 3-fluoro-2-hydroxy-5-(4-(4- (pyrrolidin-1- yl)phenyl)piperidine-1- carbonyl)benzaldehyde |
| 35 | 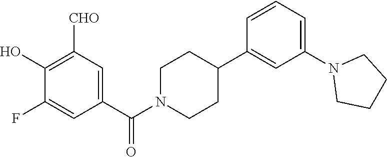 [SMILES: O=Cc1cc(C(=O)N2CCC(c3cccc(N4CCCC4)c3)CC2)cc(F)c1O] | 3-fluoro-2-hydroxy-5-(4-(3- (pyrrolidin-1- yl)phenyl)piperidine-1- carbonyl)benzaldehyde |
| 36 | 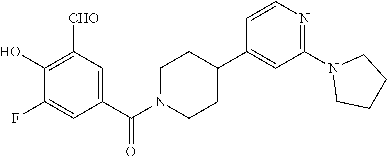 [SMILES: O=Cc1cc(C(=O)N2CCC(c3ccnc(N4CCCC4)c3)CC2)cc(F)c1O] | 3-fluoro-2-hydroxy-5-(4-(2- (pyrrolidin-1-yl)pyridin-4- yl)piperidine-1- carbonyl)benzaldehyde |
| 37 | 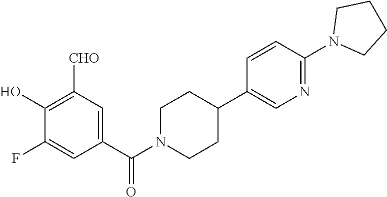 [SMILES: O=Cc1cc(C(=O)N2CCC(c3ccc(N4CCCC4)nc3)CC2)cc(F)c1O] | 3-fluoro-2-hydroxy-5-(4-(6- (pyrrolidin-1-yl)pyridin-3- yl)piperidine-1- carbonyl)benzaldehyde |
| 38 | 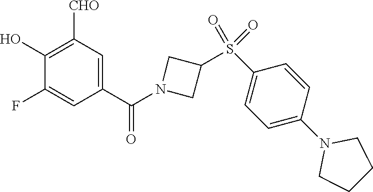 [SMILES: O=Cc1cc(C(=O)N2CC(S(=O)(=O)c3ccc(N4CCCC4)cc3)C2)cc(F)c1O] | 3-fluoro-2-hydroxy-5-(3-((4- (pyrrolidin-1- yl)phenyl)sulfonyl)azetidine- 1-carbonyl)benzaldehyde |
| 39 | 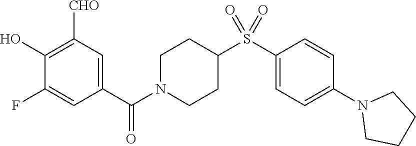 [SMILES: O=Cc1cc(C(=O)N2CCC(S(=O)(=O)c3ccc(N4CCCC4)cc3)CC2)cc(F)c1O] | 3-fluoro-2-hydroxy-5-(4-((4- (pyrrolidin-1- yl)phenyl)sulfonyl)piperidine- 1-carbonyl)benzaldehyde |
| 40 | 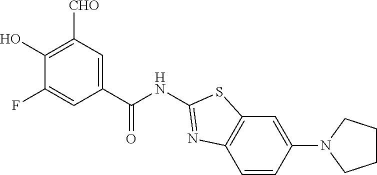 [SMILES: O=Cc1cc(C(=O)Nc2nc3ccc(N4CCCC4)cc3s2)cc(F)c1O] | 3-fluoro-5-formyl-4-hydroxy- N-(6-(pyrrolidin-1- yl)benzo[d]thiazol-2- yl)benzamide |
| 41 | 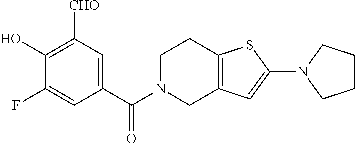 [SMILES: O=Cc1cc(C(=O)N2CCc3sc(N4CCCC4)cc3C2)cc(F)c1O] | 3-fluoro-2-hydroxy-5-(2- (pyrrolidin-1-yl)-4,5,6,7- tetrahydrothieno[3,2- c]pyridine-5- carbonyl)benzaldehyde |
| 42 | 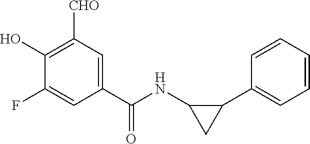 [SMILES: O=Cc1cc(C(=O)NC2CC2c2ccccc2)cc(F)c1O] | 3-fluoro-5-formyl-4-hydroxy- N-(2- phenylcyclopropyl)benzamide |
| 43 | 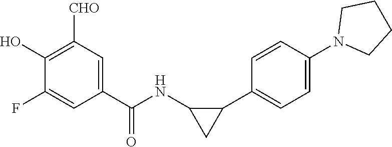 [SMILES: O=Cc1cc(C(=O)NC2CC2c2ccc(N3CCCC3)cc2)cc(F)c1O] | 3-fluoro-5-formyl-4-hydroxy- N-(2-(4-(pyrrolidin-1- yl)phenyl)cyclopropyl) benzamide |
| 44 | 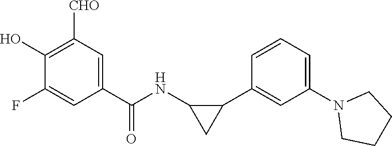 [SMILES: O=Cc1cc(C(=O)NC2CC2c2cccc(N3CCCC3)c2)cc(F)c1O] | 3-fluoro-5-formyl-4-hydroxy- N-(2-(3-(pyrrolidin-1- yl)phenyl)cyclopropyl) benzamide |
| 45 | 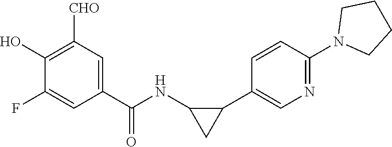 [SMILES: O=Cc1cc(C(=O)NC2CC2c2ccc(N3CCCC3)nc2)cc(F)c1O] | 3-fluoro-5-formyl-4-hydroxy- N-(2-(6-(pyrrolidin-1- yl)pyridin-3- yl)cyclopropyl)benzamide |
| 46 | 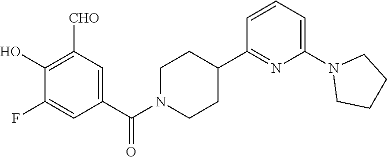 [SMILES: O=Cc1cc(C(=O)N2CCC(c3cccc(N4CCCC4)n3)CC2)cc(F)c1O] | 3-fluoro-2-hydroxy-5-(4-(6- (pyrrolidin-1-yl)pyridin-2- yl)piperidine-1- carbonyl)benzaldehyde |
| 47 | 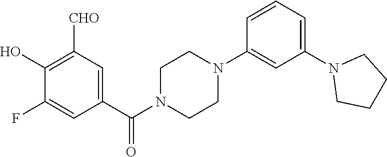 [SMILES: O=Cc1cc(C(=O)N2CCN(c3cccc(N4CCCC4)c3)CC2)cc(F)c1O] | 3-fluoro-2-hydroxy-5-(4-(3- (pyrrolidin-1- yl)phenyl)piperazine-1- carbonyl)benzaldehyde |
| 48 | 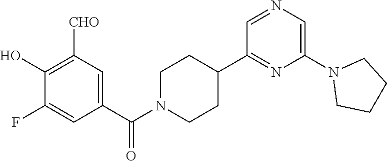 [SMILES: O=Cc1cc(C(=O)N2CCC(c3cncc(N4CCCC4)n3)CC2)cc(F)c1O] | 3-fluoro-2-hydroxy-5-(4-(6- (pyrrolidin-1-yl)pyrazin-2- yl)piperidine-1- carbonyl)benzaldehyde |
| 49 | 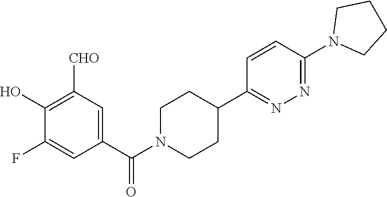 [SMILES: O=Cc1cc(C(=O)N2CCC(c3ccc(N4CCCC4)nn3)CC2)cc(F)c1O] | 3-fluoro-2-hydroxy-5-(4-(6- (pyrrolidin-1-yl)pyridazin-3- yl)piperidine-1- carbonyl)benzaldehyde |
| 50 | 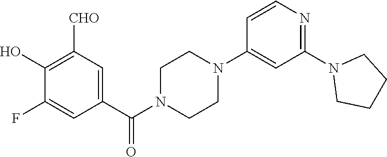 [SMILES: O=Cc1cc(C(=O)N2CCN(c3ccnc(N4CCCC4)c3)CC2)cc(F)c1O] | 3-fluoro-2-hydroxy-5-(4-(2- (pyrrolidin-1-yl)pyridin-4- yl)piperazine-1- carbonyl)benzaldehyde |
| 51 | 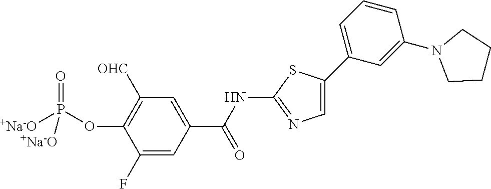 | sodium 2-((11-oxidanyl)-13- methyl)-6-fluoro-4-((5-(3- (pyrrolidin-1- yl)phenyl)thiazol-2- yl)carbamoyl)phenyl phosphate |
| 52 | 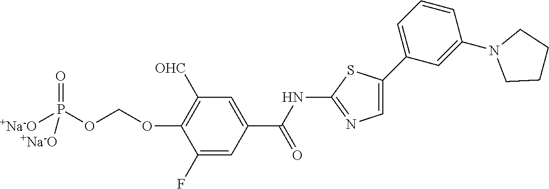 | sodium (2-fluoro-6-formyl-4- ((5-(3-(pyrrolidin-1- yl)phenyl)thiazol-2- yl)carbamoyl)phenoxy)methyl phosphate |
| 53 | 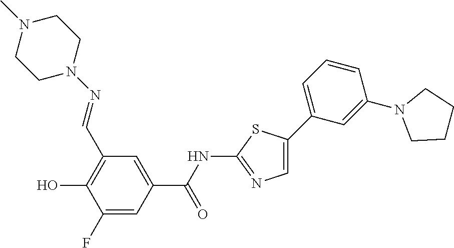 [SMILES: CN1CCN(/N=C/c2cc(C(=O)Nc3ncc(-c4cccc(N5CCCC5)c4)s3)cc(F)c2O)CC1] | (E)-3-fluoro-4-hydroxy-5-(((4- methylpiperazin-1- yl)imino)methyl)-N-(5-(3- (pyrrolidin-1- yl)phenyl)thiazol-2- yl)benzamide |
| 54 | 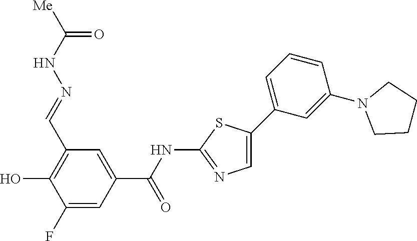 [SMILES: CC(=O)N/N=C/c1cc(C(=O)Nc2ncc(-c3cccc(N4CCCC4)c3)s2)cc(F)c1O] | (E)-3-((2- acetylhydrazono)methyl)-5- fluoro-4-hydroxy-N-(5-(3- (pyrrolidin-1- yl)phenyl)thiazol-2- yl)benzamide |
| 55 | 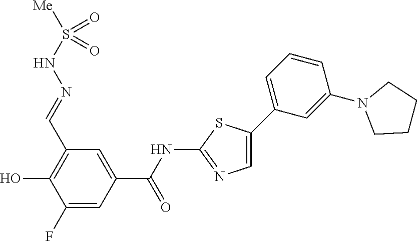 [SMILES: CS(=O)(=O)N/N=C/c1cc(C(=O)Nc2ncc(-c3cccc(N4CCCC4)c3)s2)cc(F)c1O] | (E)-3-fluoro-4-hydroxy-5-((2- (methylsulfonyl)hydrazono) methyl)-N-(5-(3-(pyrrolidin-1- yl)phenyl)thiazol-2- yl)benzamide |
| 56 | 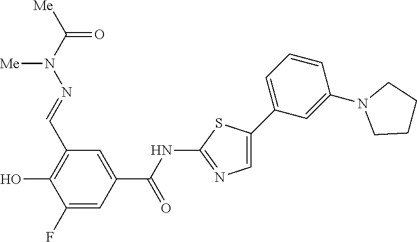 [SMILES: CC(=O)N(C)/N=C/c1cc(C(=O)Nc2ncc(-c3cccc(N4CCCC4)c3)s2)cc(F)c1O] | (E)-3-((2-acetyl-2- methylhydrazono)methyl)-5- fluoro-4-hydroxy-N-(5-(3- (pyrrolidin-1- yl)phenyl)thiazol-2- yl)benzamide |
| 57 | 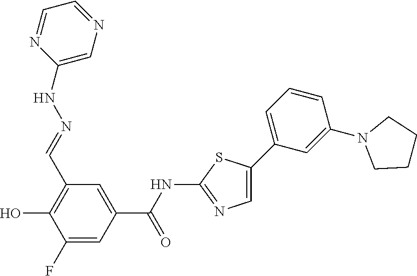 [SMILES: O=C(Nc1ncc(-c2cccc(N3CCCC3)c2)s1)c1cc(F)c(O)c(/C=N/Nc2cnccn2)c1] | (E)-3-fluoro-4-hydroxy-5-((2- (pyrazin-2- yl)hydrazono)methyl)-N-(5- (3-(pyrrolidin-1- yl)phenyl)thiazol-2- yl)benzamide |
| 58 | 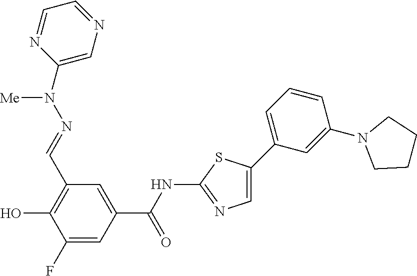 [SMILES: CN(/N=C/c1cc(C(=O)Nc2ncc(-c3cccc(N4CCCC4)c3)s2)cc(F)c1O)c1cnccn1] | (E)-3-fluoro-4-hydroxy-5-((2- methyl-2-(pyrazin-2- yl)hydrazono)methyl)-N-(5- (3-(pyrrolidin-1- yl)phenyl)thiazol-2- yl)benzamide |
| 59 | 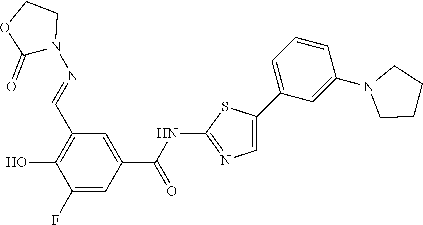 [SMILES: O=C(Nc1ncc(-c2cccc(N3CCCC3)c2)s1)c1cc(F)c(O)c(/C=N/N2CCOC2=O)c1] | (E)-3-fluoro-4-hydroxy-5-(((2- oxooxazolidin-3- yl)imino)methyl)-N-(5-(3- (pyrrolidin-1- yl)phenyl)thiazol-2- yl)benzamide |
| 60 | 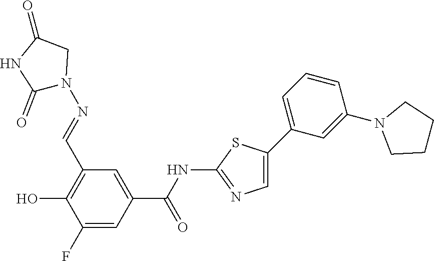 [SMILES: O=C1CN(/N=C/c2cc(C(=O)Nc3ncc(-c4cccc(N5CCCC5)c4)s3)cc(F)c2O)C(=O)N1] | (E)-3-(((2,4- dioxoimidazolidin-1- yl)imino)methyl)-5-fluoro-4- hydroxy-N-(5-(3-(pyrrolidin- 1-yl)phenyl)thiazol-2- yl)benzamide |
| 61 | 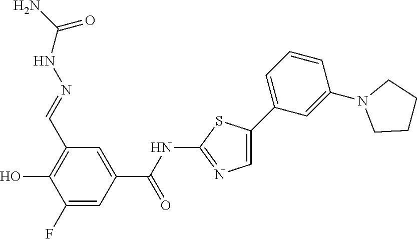 [SMILES: NC(=O)N/N=C/c1cc(C(=O)Nc2ncc(-c3cccc(N4CCCC4)c3)s2)cc(F)c1O] | (E)-2-(3-fluoro-2-hydroxy-5- ((5-(3-(pyrrolidin-1- yl)phenyl)thiazol-2- yl)carbamoyl)benzylidene) hydrazine-1-carboxamide |
| 62 | 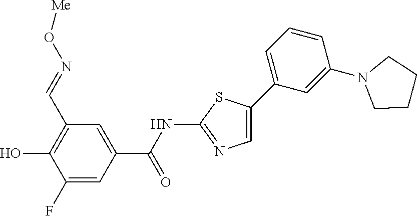 [SMILES: CO/N=C/c1cc(C(=O)Nc2ncc(-c3cccc(N4CCCC4)c3)s2)cc(F)c1O] | (E)-3-fluoro-4-hydroxy-5- ((methoxyimino)methyl)-N- (5-(3-(pyrrolidin-1- yl)phenyl)thiazol-2- yl)benzamide |
| 63 | 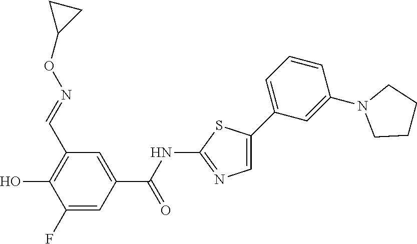 [SMILES: O=C(Nc1ncc(-c2cccc(N3CCCC3)c2)s1)c1cc(F)c(O)c(/C=N/OC2CC2)c1] | (E)-3- ((cyclopropoxyimino)methyl)- 5-fluoro-4-hydroxy-N-(5-(3- (pyrrolidin-1- yl)phenyl)thiazol-2- yl)benzamide |
| 64 | 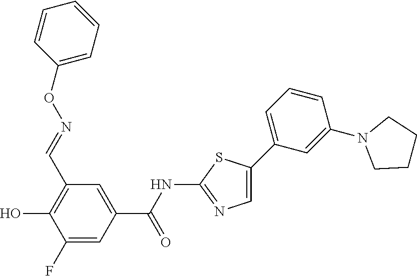 [SMILES: O=C(Nc1ncc(-c2cccc(N3CCCC3)c2)s1)c1cc(F)c(O)c(/C=N/Oc2ccccc2)c1] | (E)-3-fluoro-4-hydroxy-5- ((phenoxyimino)methyl)-N-(5- (3-(pyrrolidin-1- yl)phenyl)thiazol-2- yl)benzamide |
| 65 | 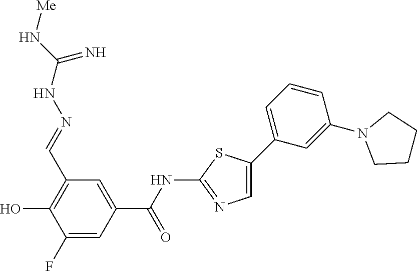 [SMILES: CNC(=N)N/N=C/c1cc(C(=O)Nc2ncc(-c3cccc(N4CCCC4)c3)s2)cc(F)c1O] | (E)-3-fluoro-4-hydroxy-5-((2- (N-methylcarbamimidoyl) hydrazono) methyl)-N-(5-(3- (pyrrolidin-1- yl)phenyl)thiazol-2- yl)benzamide |
| 66 | 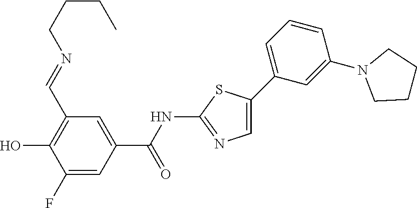 [SMILES: CCCC/N=C/c1cc(C(=O)Nc2ncc(-c3cccc(N4CCCC4)c3)s2)cc(F)c1O] | (E)-3-((butylimino)methyl)-5- fluoro-4-hydroxy-N-(5-(3- (pyrrolidin-1- yl)phenyl)thiazol-2- yl)benzamide |
| 67 | 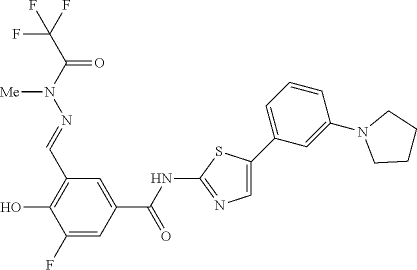 [SMILES: CN(/N=C/c1cc(C(=O)Nc2ncc(-c3cccc(N4CCCC4)c3)s2)cc(F)c1O)C(=O)C(F)(F)F] | (E)-3-fluoro-4-hydroxy-5-((2- methyl-2-(2,2,2-trifluoroacetyl) hydrazono)methyl)- N-(5-(3-(pyrrolidin-1- yl)phenyl)thiazol-2- yl)benzamide |
| 68 | 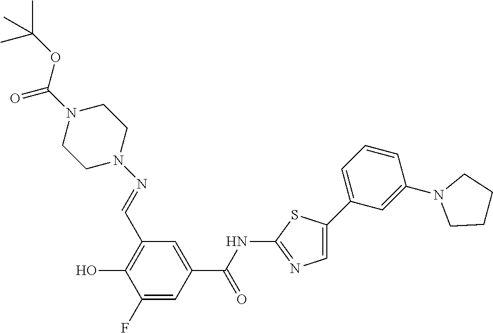 [SMILES: CC(C)(C)OC(=O)N1CCN(/N=C/c2cc(C(=O)Nc3ncc(-c4cccc(N5CCCC5)c4)s3)cc(F)c2O)CC1] | tert-butyl (E)-4-((3-fluoro-2- hydroxy-5-((5-(3-(pyrrolidin- 1-yl)phenyl)thiazol-2- yl)carbamoyl)benzylidene) amino)piperazine-1-carboxylate |
| 69 | 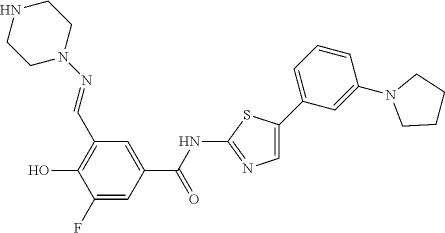 [SMILES: O=C(Nc1ncc(-c2cccc(N3CCCC3)c2)s1)c1cc(F)c(O)c(/C=N/N2CCNCC2)c1] | (E)-3-fluoro-4-hydroxy-5- ((piperazin-1-ylimino)methyl)- N-(5-(3-(pyrrolidin-1- yl)phenyl)thiazol-2- yl)benzamide |
| 70 | 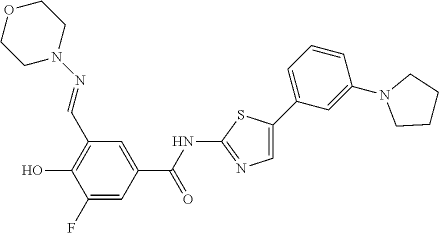 [SMILES: O=C(Nc1ncc(-c2cccc(N3CCCC3)c2)s1)c1cc(F)c(O)c(/C=N/N2CCOCC2)c1] | (E)-3-fluoro-4-hydroxy-5- ((morpholinoimino)methyl)- N-(5-(3-(pyrrolidin-1- yl)phenyl)thiazol-2- yl)benzamide |
| 71 | 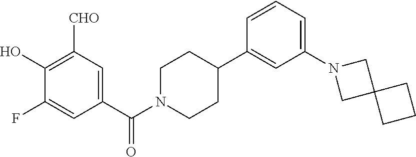 [SMILES: O=Cc1cc(C(=O)N2CCC(c3cccc(N4CC5(CCC5)C4)c3)CC2)cc(F)c1O] | 5-(4-(3-(2- azaspiro[3.3]heptan-2- yl)phenyl)piperidine-1- carbonyl)-3-fluoro-2- hydroxybenzaldehyde |
| 72 | 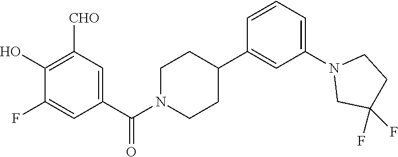 [SMILES: O=Cc1cc(C(=O)N2CCC(c3cccc(N4CCC(F)(F)C4)c3)CC2)cc(F)c1O] | 5-(4-(3-(3,3- difluoropyrrolidin-1- yl)phenyl)piperidine-1- carbonyl)-3-fluoro-2- hydroxybenzaldehyde |
| 73 | 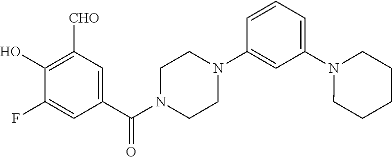 [SMILES: O=Cc1cc(C(=O)N2CCN(c3cccc(N4CCCCC4)c3)CC2)cc(F)c1O] | 3-fluoro-2-hydroxy-5-(4-(3- (piperidin-1- yl)phenyl)piperazine-1- carbonyl)benzaldehyde |
| 74 | 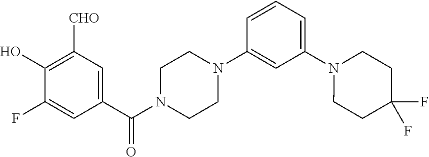 [SMILES: O=Cc1cc(C(=O)N2CCN(c3cccc(N4CCC(F)(F)CC4)c3)CC2)cc(F)c1O] | 5-(4-(3-(4,4-difluoropiperidin- 1-yl)phenyl)piperazine-1- carbonyl)-3-fluoro-2- hydroxybenzaldehyde |
| 75 | 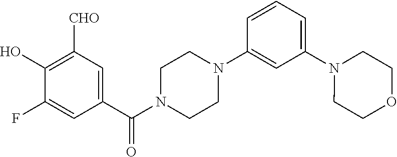 [SMILES: O=Cc1cc(C(=O)N2CCN(c3cccc(N4CCOCC4)c3)CC2)cc(F)c1O] | 3-fluoro-2-hydroxy-5-(4-(3- morpholinophenyl)piperazine- 1-carbonyl)benzaldehyde |
| 76 | 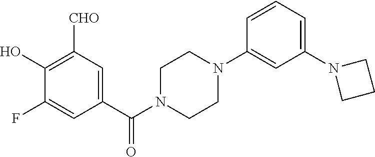 [SMILES: O=Cc1cc(C(=O)N2CCN(c3cccc(N4CCC4)c3)CC2)cc(F)c1O] | 5-(4-(3-(azetidin-1- yl)phenyl)piperazine-1- carbonyl)-3-fluoro-2- hydroxybenzaldehyde |
| 77 | 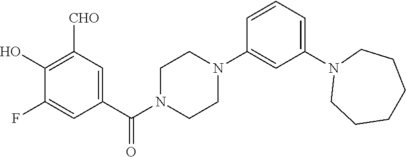 [SMILES: O=Cc1cc(C(=O)N2CCN(c3cccc(N4CCCCCC4)c3)CC2)cc(F)c1O] | 5-(4-(3-(azepan-1- yl)phenyl)piperazine-1- carbonyl)-3-fluoro-2- hydroxybenzaldehyde |
| 78 | 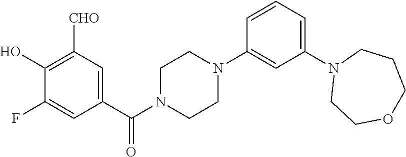 [SMILES: O=Cc1cc(C(=O)N2CCN(c3cccc(N4CCCOCC4)c3)CC2)cc(F)c1O] | 5-(4-(3-(1,4-oxazepan-4- yl)phenyl)piperazine-1- carbonyl)-3-fluoro-2- hydroxybenzaldehyde |
| 79 | 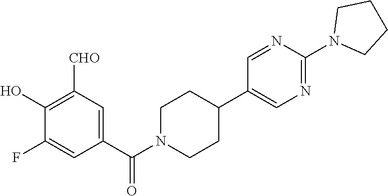 [SMILES: O=Cc1cc(C(=O)N2CCC(c3cnc(N4CCCC4)nc3)CC2)cc(F)c1O] | 3-fluoro-2-hydroxy-5-(4-(2- (pyrrolidin-1-yl)pyrimidin-5- yl)piperidine-1- carbonyl)benzaldehyde |
| 80 | 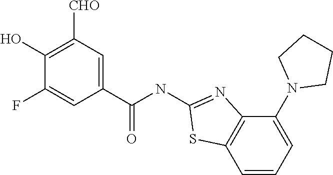 [SMILES: O=Cc1cc(C(=O)Nc2nc3c(N4CCCC4)cccc3s2)cc(F)c1O] | 3-fluoro-5-formyl-4-hydroxy- N-(4-(pyrrolidin-1- yl)benzo[d]thiazol-2- yl)benzamide |
| 81 | 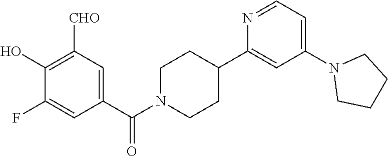 [SMILES: O=Cc1cc(C(=O)N2CCC(c3cc(N4CCCC4)ccn3)CC2)cc(F)c1O] | 3-fluoro-2-hydroxy-5-(4-(4- (pyrrolidin-1-yl)pyridin-2- yl)piperidine-1- carbonyl)benzaldehyde |
| 82 | 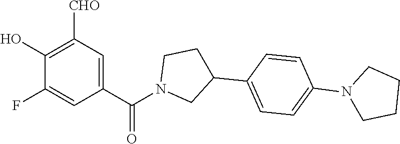 [SMILES: O=Cc1cc(C(=O)N2CCC(c3ccc(N4CCCC4)cc3)C2)cc(F)c1O] | 3-fluoro-2-hydroxy-5-(3-(4- (pyrrolidin-1- yl)phenyl)pyrrolidine-1- carbonyl)benzaldehyde |
| 83 | 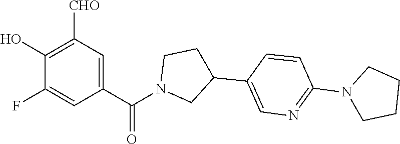 [SMILES: O=Cc1cc(C(=O)N2CCC(c3ccc(N4CCCC4)nc3)C2)cc(F)c1O] | 3-fluoro-2-hydroxy-5-(3-(6- (pyrrolidin-1-yl)pyridin-3- yl)pyrrolidine-1- carbonyl)benzaldehyde |
| 84 | 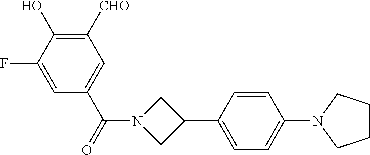 [SMILES: O=Cc1cc(C(=O)N2CC(c3ccc(N4CCCC4)cc3)C2)cc(F)c1O] | 3-fluoro-2-hydroxy-5-(3-(4- (pyrrolidin-1- yl)phenyl)azetidine-1- carbonyl)benzaldehyde |
| 85 | 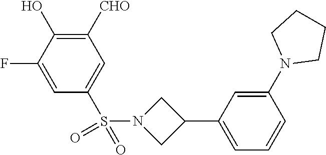 [SMILES: O=Cc1cc(S(=O)(=O)N2CC(c3cccc(N4CCCC4)c3)C2)cc(F)c1O] | 3-fluoro-2-hydroxy-5-((1-(3- (pyrrolidin-1- yl)phenyl)azetidin-3- yl)sulfonyl)benzaldehyde |
| 86 | 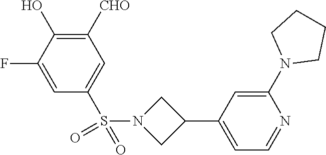 [SMILES: O=Cc1cc(S(=O)(=O)N2CC(c3ccnc(N4CCCC4)c3)C2)cc(F)c1O] | 3-fluoro-2-hydroxy-5-((1-(2- (pyrrolidin-1-yl)pyridin-4- yl)azetidin-3- yl)sulfonyl)benzaldehyde |
| 87 | 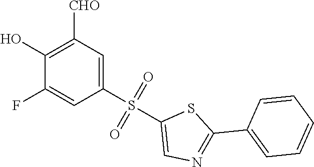 [SMILES: O=Cc1cc(S(=O)(=O)c2cnc(-c3ccccc3)s2)cc(F)c1O] | 3-fluoro-2-hydroxy-5-((2- phenylthiazol-5- yl)sulfonyl)benzaldehyde |
| 88 | 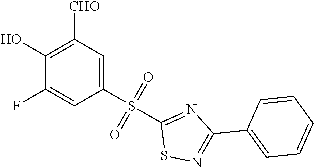 [SMILES: O=Cc1cc(S(=O)(=O)c2nc(-c3ccccc3)ns2)cc(F)c1O] | -fluoro-2-hydroxy-5-((3- phenyl-1,2,4-thiadiazol-5- yl)sulfonyl)benzaldehyde |
| 89 | 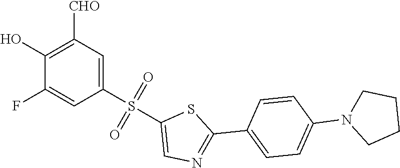 [SMILES: O=Cc1cc(S(=O)(=O)c2cnc(-c3ccc(N4CCCC4)cc3)s2)cc(F)c1O] | 3-fluoro-2-hydroxy-5-((2-(4- (pyrrolidin-1- yl)phenyl)thiazol-5- yl)sulfonyl)benzaldehyde |
| 90 | 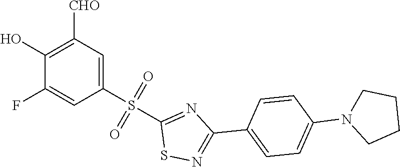 [SMILES: O=Cc1cc(S(=O)(=O)c2nc(-c3ccc(N4CCCC4)cc3)ns2)cc(F)c1O] | 3-fluoro-2-hydroxy-5-((3-(4- (pyrrolidin-1-yl)phenyl)-1,2,4- thiadiazol-5- yl)sulfonyl)benzaldehyde |
| 91 | 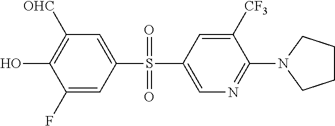 [SMILES: O=Cc1cc(S(=O)(=O)c2cnc(N3CCCC3)c(C(F)(F)F)c2)cc(F)c1O] | 3-fluoro-2-hydroxy-5-((6- (pyrrolidin-1-yl)-5- (trifluoromethyl)pyridin-3- yl)sulfonyl)benzaldehyde |
| 92 | 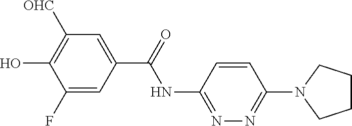 [SMILES: O=Cc1cc(C(=O)Nc2ccc(N3CCCC3)nn2)cc(F)c1O] | 3-fluoro-5-formyl-4-hydroxy- N-(6-(pyrrolidin-1- yl)pyridazin-3-yl)benzamide |
| 93 | 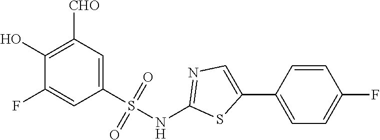 [SMILES: O=Cc1cc(S(=O)(=O)Nc2ncc(-c3ccc(F)cc3)s2)cc(F)c1O] | 3-fluoro-N-(5-(4- fluorophenyl)thiazol-2-yl)-5- formyl-4- hydroxybenzenesulfonamide |
| 94 | 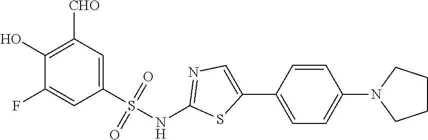 [SMILES: O=Cc1cc(S(=O)(=O)Nc2ncc(-c3ccc(N4CCCC4)cc3)s2)cc(F)c1O] | 3-fluoro-5-formyl-4-hydroxy- N-(5-(4-(pyrrolidin-1- yl)phenyl)thiazol-2- yl)benzenesulfonamide |
| 95 | 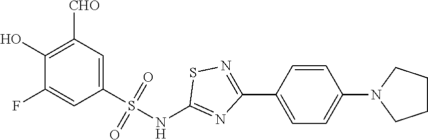 [SMILES: O=Cc1cc(S(=O)(=O)Nc2nc(-c3ccc(N4CCCC4)cc3)ns2)cc(F)c1O] | 3-fluoro-5-formyl-4-hydroxy- N-(3-(4-(pyrrolidin-1- yl)phenyl)-1,2,4-thiadiazol-5- yl)benzenesulfonamide |
| 96 | 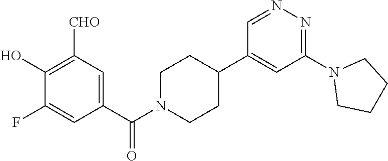 [SMILES: O=Cc1cc(C(=O)N2CCC(c3cnnc(N4CCCC4)c3)CC2)cc(F)c1O] | 3-fluoro-2-hydroxy-5-(4-(6- (pyrrolidin-1-yl)pyridazin-4- yl)piperidine-1- carbonyl)benzaldehyde |
| 97 | 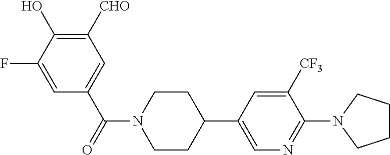 [SMILES: O=Cc1cc(C(=O)N2CCC(c3cnc(N4CCCC4)c(C(F)(F)F)c3)CC2)cc(F)c1O] | 3-fluoro-2-hydroxy-5-(4-(6- (pyrrolidin-1-yl)-5- (trifluoromethyl)pyridin-3- yl)piperidine-1- carbonyl)benzaldehyde |
| 98 | 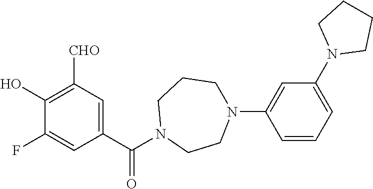 [SMILES: O=Cc1cc(C(=O)N2CCCN(c3cccc(N4CCCC4)c3)CC2)cc(F)c1O] | 3-fluoro-2-hydroxy-5-(4-(3- (pyrrolidin-1-yl)phenyl)-1,4- diazepane-1- carbonyl)benzaldehyde |
| 99 | 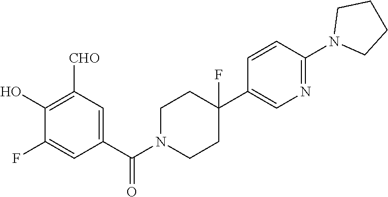 [SMILES: O=Cc1cc(C(=O)N2CCC(F)(c3ccc(N4CCCC4)nc3)CC2)cc(F)c1O] | 3-fluoro-5-(4-fluoro-4-(6- (pyrrolidin-1-yl)pyridin-3- yl)piperidine-1-carbonyl)-2- hydroxybenzaldehyde |
| 100 | 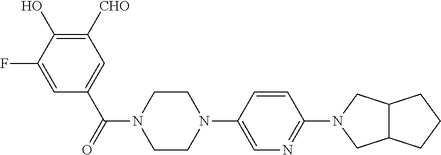 [SMILES: O=Cc1cc(C(=O)N2CCN(c3ccc(N4CC5CCCC5C4)nc3)CC2)cc(F)c1O] | 3-fluoro-5-(4-(6- (hexahydrocyclopenta[c]pyrrol- 2(1H)-yl)pyridin-3- yl)piperazine-1-carbonyl)-2- hydroxybenzaldehyde |
| 101 | 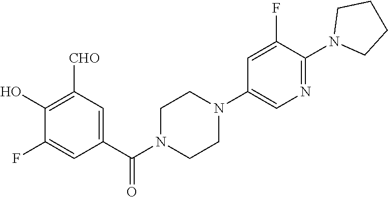 [SMILES: O=Cc1cc(C(=O)N2CCN(c3cnc(N4CCCC4)c(F)c3)CC2)cc(F)c1O] | 3-fluoro-5-(4-(5-fluoro-6- (pyrrolidin-1-yl)pyridin-3- yl)piperazine-1-carbonyl)-2- hydroxybenzaldehyde |
| 102 | 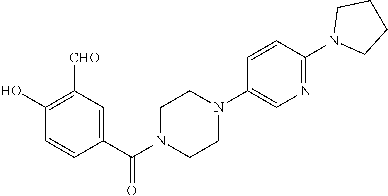 [SMILES: O=Cc1cc(C(=O)N2CCN(c3ccc(N4CCCC4)nc3)CC2)ccc1O] | 2-hydroxy-5-(4-(6-(pyrrolidin- 1-yl)pyridin-3-yl)piperazine-1- carbonyl)benzaldehyde |
| 103 | 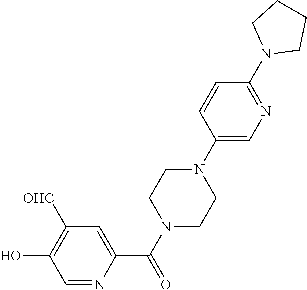 [SMILES: O=Cc1cc(C(=O)N2CCN(c3ccc(N4CCCC4)nc3)CC2)ncc1O] | 5-hydroxy-2-(4-(6-(pyrrolidin- 1-yl)pyridin-3-yl)piperazine-1- carbonyl)isonicotinaldehyde |
| 104 | 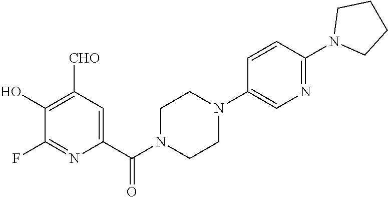 [SMILES: O=Cc1cc(C(=O)N2CCN(c3ccc(N4CCCC4)nc3)CC2)nc(F)c1O] | 3-hydroxy-2-methyl-6-(4-(6- (pyrrolidin-1-yl)pyridin-3- yl)piperazine-1- carbonyl)isonicotinaldehyde |
| 105 | 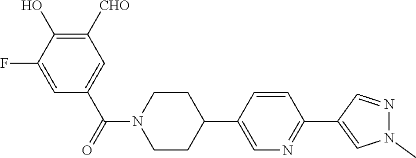 [SMILES: Cn1cc(-c2ccc(C3CCN(C(=O)c4cc(F)c(O)c(C=O)c4)CC3)cn2)cn1] | 3-fluoro-2-hydroxy-5-(4-(6-(1- methyl-1H-pyrazol-4- yl)pyridin-3-yl)piperidine-1- carbonyl)benzaldehyde |
| 106 | 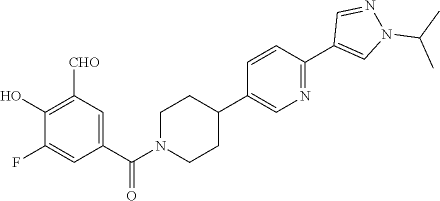 [SMILES: CC(C)n1cc(-c2ccc(C3CCN(C(=O)c4cc(F)c(O)c(C=O)c4)CC3)cn2)cn1] | 3-fluoro-2-hydroxy-5-(4-(6-(1- isopropyl-1H-pyrazol-4- yl)pyridin-3-yl)piperidine-1- carbonyl)benzaldehyde |
| 107 | 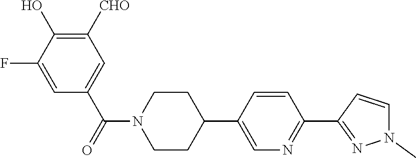 [SMILES: Cn1ccc(-c2ccc(C3CCN(C(=O)c4cc(F)c(O)c(C=O)c4)CC3)cn2)n1] | 3-fluoro-2-hydroxy-5-(4-(6-(1- methyl-1H-pyrazol-3- yl)pyridin-3-yl)piperidine-1- carbonyl)benzaldehyde |
| 108 | 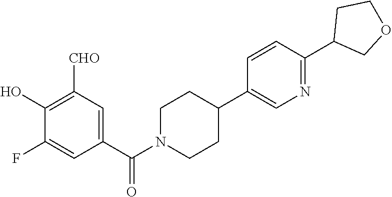 [SMILES: O=Cc1cc(C(=O)N2CCC(c3ccc(C4CCOC4)nc3)CC2)cc(F)c1O] | 3-fluoro-2-hydroxy-5-(4-(6- (tetrahydrofuran-3-yl)pyridin- 3-yl)piperidine-1- carbonyl)benzaldehyde |
| 109 | 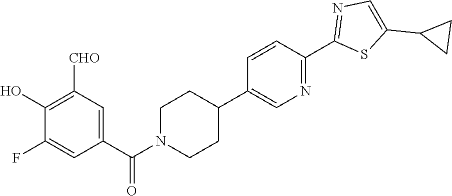 [SMILES: O=Cc1cc(C(=O)N2CCC(c3ccc(-c4ncc(C5CC5)s4)nc3)CC2)cc(F)c1O] | 5-(4-(6-(5-cyclopropylthiazol- 2-yl)pyridin-3-yl)piperidine-1- carbonyl)-3-fluoro-2- hydroxybenzaldehyde |
| 110 | 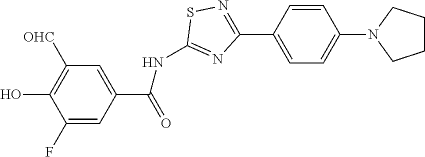 [SMILES: O=Cc1cc(C(=O)Nc2nc(-c3ccc(N4CCCC4)cc3)ns2)cc(F)c1O] | 3-fluoro-5-formyl-4-hydroxy- N-(3-(4-(pyrrolidin-1- yl)phenyl)-1,2,4-thiadiazol-5- yl)benzamide |
| 111 | 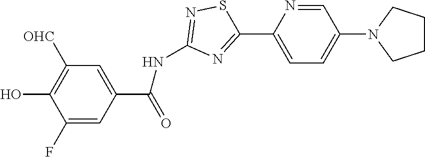 [SMILES: O=Cc1cc(C(=O)Nc2nsc(-c3ccc(N4CCCC4)cn3)n2)cc(F)c1O] | 3-fluoro-5-formyl-4-hydroxy- N-(5-(5-(pyrrolidin-1- yl)pyridin-2-yl)-1,2,4- thiadiazol-3-yl)benzamide |
| 112 | 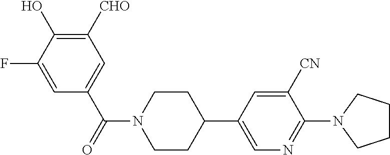 [SMILES: N#Cc1cc(C2CCN(C(=O)c3cc(F)c(O)c(C=O)c3)CC2)cnc1N1CCCC1] | 5-(1-(3-fluoro-5-formyl-4- hydroxybenzoyl)piperidin-4- yl)-2-(pyrrolidin-1- yl)nicotinonitrile |
| 113 | 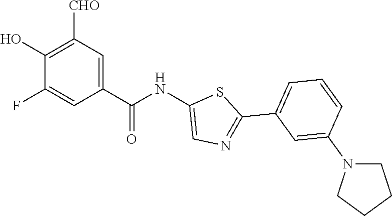 [SMILES: O=Cc1cc(C(=O)Nc2cnc(-c3cccc(N4CCCC4)c3)s2)cc(F)c1O] | 3-fluoro-5-formyl-4-hydroxy- N-(2-(3-(pyrrolidin-1- yl)phenyl)thiazol-5- yl)benzamide |
| 114 | 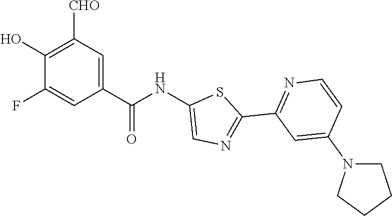 [SMILES: O=Cc1cc(C(=O)Nc2cnc(-c3cc(N4CCCC4)ccn3)s2)cc(F)c1O] | 3-fluoro-5-formyl-4-hydroxy- N-(2-(4-(pyrrolidin-1- yl)pyridin-2-yl)thiazol-5- yl)benzamide |
| 115 | 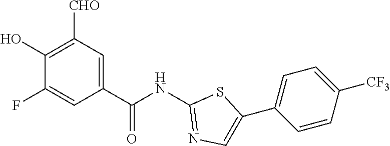 [SMILES: O=Cc1cc(C(=O)Nc2ncc(-c3ccc(C(F)(F)F)cc3)s2)cc(F)c1O] | 3-fluoro-5-formyl-4-hydroxy- N-(5-(4- (trifluoromethyl)phenyl) thiazol-2-yl)benzamide |
| 116 | 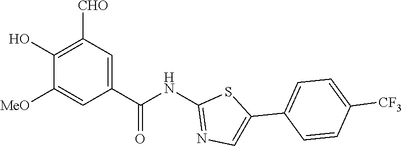 [SMILES: COc1cc(C(=O)Nc2ncc(-c3ccc(C(F)(F)F)cc3)s2)cc(C=O)c1O] | 3-formyl-4-hydroxy-5- methoxy-N-(5-(4- (trifluoromethyl)phenyl) thiazol-2-yl)benzamide |
| 117 | 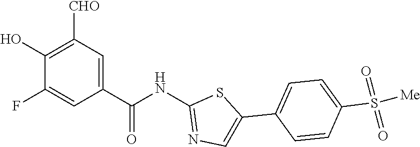 [SMILES: CS(=O)(=O)c1ccc(-c2cnc(NC(=O)c3cc(F)c(O)c(C=O)c3)s2)cc1] | 3-fluoro-5-formyl-4-hydroxy- N-(5-(4- (methylsulfonyl)phenyl) thiazol-2-yl)benzamide |
| 118 | 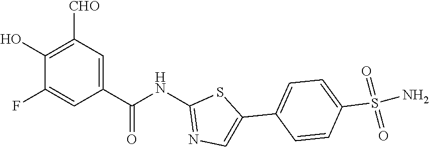 [SMILES: NS(=O)(=O)c1ccc(-c2cnc(NC(=O)c3cc(F)c(O)c(C=O)c3)s2)cc1] | 3-fluoro-5-formyl-4-hydroxy- N-(5-(4- sulfamoylphenyl)thiazol-2- yl)benzamide |
| 119 | 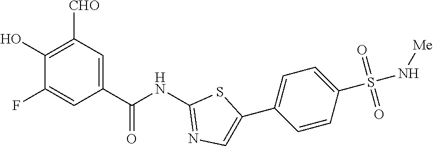 [SMILES: CNS(=O)(=O)c1ccc(-c2cnc(NC(=O)c3cc(F)c(O)c(C=O)c3)s2)cc1] | 3-fluoro-5-formyl-4-hydroxy- N-(5-(4-(N- methylsulfamoyl)phenyl) thiazol-2-yl)benzamide |
| 120 | 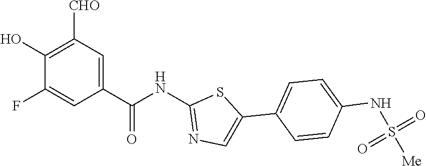 [SMILES: CS(=O)(=O)Nc1ccc(-c2cnc(NC(=O)c3cc(F)c(O)c(C=O)c3)s2)cc1] | 3-fluoro-5-formyl-4-hydroxy- N-(5-(4- (methylsulfonamido)phenyl) thiazol-2-yl)benzamide |
| 121 | 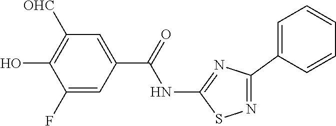 [SMILES: O=Cc1cc(C(=O)Nc2nc(-c3ccccc3)ns2)cc(F)c1O] | 3-fluoro-5-formyl-4-hydroxy- N-(3-phenyl-1,2,4-thiadiazol- 5-yl)benzamide |
| 122 | 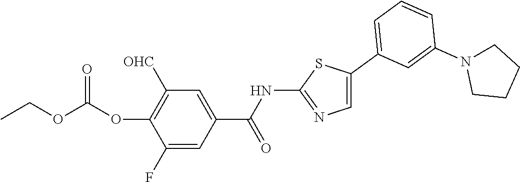 [SMILES: CCOC(=O)Oc1c(F)cc(C(=O)Nc2ncc(-c3cccc(N4CCCC4)c3)s2)cc1C=O] | ethyl (2-fluoro-6-formyl-4-((5- (3-(pyrrolidin-1- yl)phenyl)thiazol-2- yl)carbamoyl)phenyl) carbonate |
| 123 | 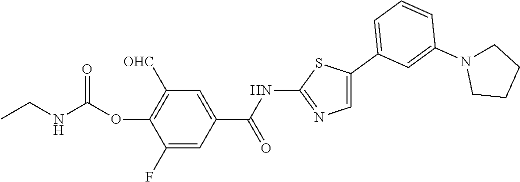 [SMILES: CCNC(=O)Oc1c(F)cc(C(=O)Nc2ncc(-c3cccc(N4CCCC4)c3)s2)cc1C=O] | 2-fluoro-6-formyl-4-((5-(3- (pyrrolidin-1- yl)phenyl)thiazol-2- yl)carbamoyl)phenyl ethylcarbamate |
| 124 | 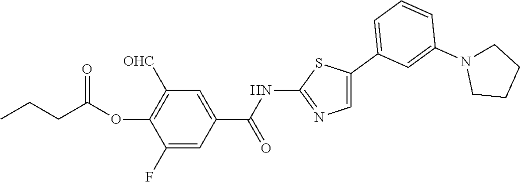 [SMILES: CCCC(=O)Oc1c(F)cc(C(=O)Nc2ncc(-c3cccc(N4CCCC4)c3)s2)cc1C=O] | 2-fluoro-6-formyl-4-((5-(3- (pyrrolidin-1- yl)phenyl)thiazol-2- yl)carbamoyl)phenyl butyrate |
| 125 | 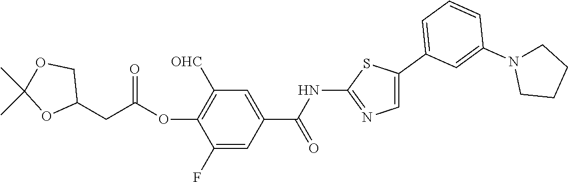 [SMILES: CC1(C)OCC(CC(=O)Oc2c(F)cc(C(=O)Nc3ncc(-c4cccc(N5CCCC5)c4)s3)cc2C=O)O1] | 2-fluoro-6-formyl-4-((5-(3- (pyrrolidin-1- yl)phenyl)thiazol-2- yl)carbamoyl)phenyl 2-(2,2- dimethyl-1,3-dioxolan-4- yl)acetate |
| 126 | 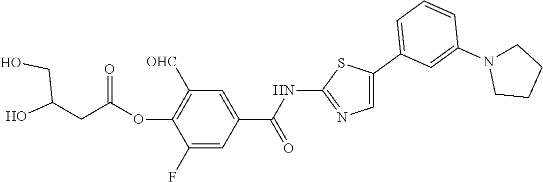 [SMILES: O=Cc1cc(C(=O)Nc2ncc(-c3cccc(N4CCCC4)c3)s2)cc(F)c1OC(=O)CC(O)CO] | 2-fluoro-6-formyl-4-((5-(3- (pyrrolidin-1- yl)phenyl)thiazol-2- yl)carbamoyl)phenyl 3,4- dihydroxybutanoate |
| 127 | 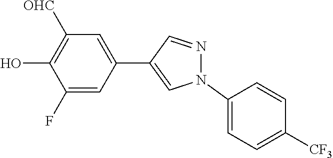 [SMILES: O=Cc1cc(-c2cnn(-c3ccc(C(F)(F)F)cc3)c2)cc(F)c1O] | 3-fluoro-2-hydroxy-5-(1-(4- (trifluoromethyl)phenyl)-1H- pyrazol-4-yl)benzaldehyde |
| 128 | 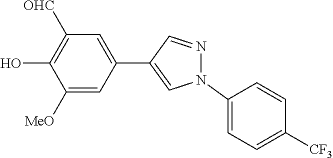 [SMILES: COc1cc(-c2cnn(-c3ccc(C(F)(F)F)cc3)c2)cc(C=O)c1O] | 2-hydroxy-3-methoxy-5-(1-(4- (trifluoromethyl)phenyl)-1H- pyrazol-4-yl)benzaldehyde |
| 129 | 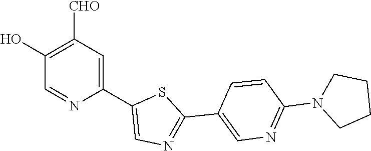 [SMILES: O=Cc1cc(-c2cnc(-c3ccc(N4CCCC4)nc3)s2)ncc1O] | 5-hydroxy-2-(2-(6-(pyrrolidin- 1-yl)pyridin-3-yl)thiazol-5- yl)isonicotinaldehyde |
| 130 | 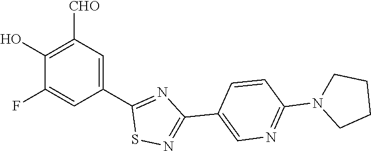 [SMILES: O=Cc1cc(-c2nc(-c3ccc(N4CCCC4)nc3)ns2)cc(F)c1O] | 3-fluoro-2-hydroxy-5-(3-(6- (pyrrolidin-1-yl)pyridin-3-yl)- 1,2,4-thiadiazol-5- yl)benzaldehyde |
| 131 | 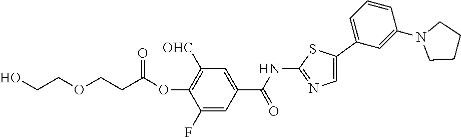 [SMILES: O=Cc1cc(C(=O)Nc2ncc(-c3cccc(N4CCCC4)c3)s2)cc(F)c1OC(=O)CCOCCO] | 2-fluoro-6-formyl-4-((5-(3- (pyrrolidin-1- yl)phenyl)thiazol-2- yl)carbamoyl)phenyl 3-(2- hydroxyethoxy)propanoate |
| 132 | 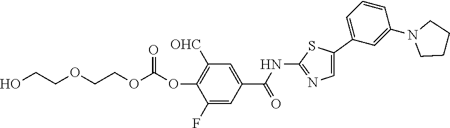 [SMILES: O=Cc1cc(C(=O)Nc2ncc(-c3cccc(N4CCCC4)c3)s2)cc(F)c1OC(=O)OCCOCCO] | 2-fluoro-6-formyl-4-((5-(3- (pyrrolidin-1- yl)phenyl)thiazol-2- yl)carbamoyl)phenyl (2-(2- hydroxyethoxy)ethyl) carbonate |
| 133 | 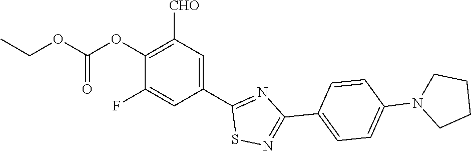 [SMILES: CCOC(=O)Oc1c(F)cc(-c2nc(-c3ccc(N4CCCC4)cc3)ns2)cc1C=O] | ethyl (2-fluoro-6-formyl-4-(3- (4-(pyrrolidin-1-yl)phenyl)- 1,2,4-thiadiazol-5-yl)phenyl) carbonate |
| 134 | 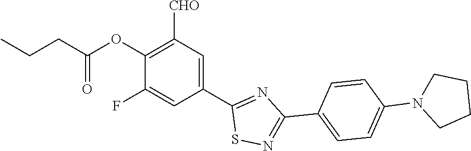 [SMILES: CCCC(=O)Oc1c(F)cc(-c2nc(-c3ccc(N4CCCC4)cc3)ns2)cc1C=O] | 2-fluoro-6-formyl-4-(3-(4- (pyrrolidin-1-yl)phenyl)-1,2,4- thiadiazol-5-yl)phenyl butyrate |
| 135 | 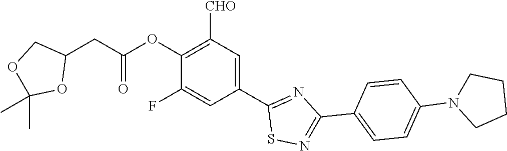 [SMILES: CC1(C)OCC(CC(=O)Oc2c(F)cc(-c3nc(-c4ccc(N5CCCC5)cc4)ns3)cc2C=O)O1] | 2-fluoro-6-formyl-4-(3-(4- (pyrrolidin-1-yl)phenyl)-1,2,4- thiadiazol-5-yl)phenyl 2-(2,2- dimethyl-1,3-dioxolan-4- yl)acetate |
| 136 | 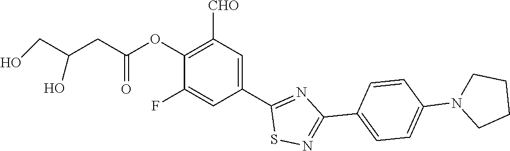 [SMILES: O=Cc1cc(-c2nc(-c3ccc(N4CCCC4)cc3)ns2)cc(F)c1OC(=O)CC(O)CO] | 2-fluoro-6-formyl-4-(3-(4- (pyrrolidin-1-yl)phenyl)-1,2,4- thiadiazol-5-yl)phenyl 3,4- dihydroxybutanoate |
| 137 | 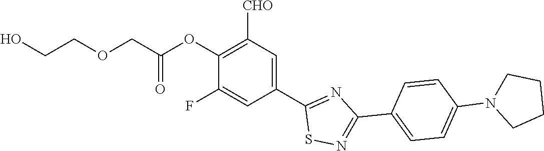 [SMILES: O=Cc1cc(-c2nc(-c3ccc(N4CCCC4)cc3)ns2)cc(F)c1OC(=O)COCCO] | 2-fluoro-6-formyl-4-(3-(4- (pyrrolidin-1-yl)phenyl)-1,2,4- thiadiazol-5-yl)phenyl 2-(2- hydroxyethoxy)acetate |
| 138 | 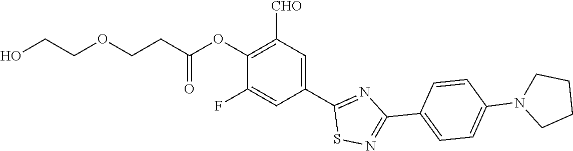 [SMILES: O=Cc1cc(-c2nc(-c3ccc(N4CCCC4)cc3)ns2)cc(F)c1OC(=O)CCOCCO] | 2-fluoro-6-formyl-4-(3-(4- (pyrrolidin-1-yl)phenyl)-1,2,4- thiadiazol-5-yl)phenyl 3-(2- hydroxyethoxy)propanoate |
| 139 | 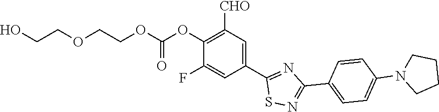 [SMILES: O=Cc1cc(-c2nc(-c3ccc(N4CCCC4)cc3)ns2)cc(F)c1OC(=O)OCCOCCO] | 2-fluoro-6-formyl-4-(3-(4- (pyrrolidin-1-yl)phenyl)-1,2,4- thiadiazol-5-yl)phenyl (2-(2- hydroxyethoxy)ethyl) carbonate |
| 140 | 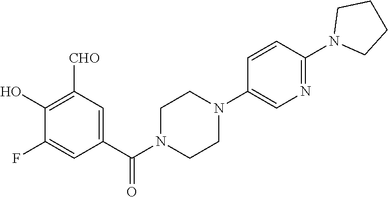 [SMILES: O=Cc1cc(C(=O)N2CCN(c3ccc(N4CCCC4)nc3)CC2)cc(F)c1O] | 3-fluoro-2-hydroxy-5-(4-(6- (pyrrolidin-1-yl)pyridin-3- yl)piperazine-1- carbonyl)benzaldehyde |
| 141 | 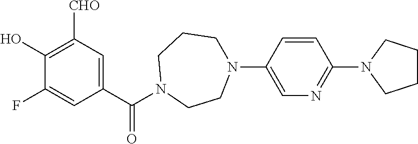 [SMILES: O=Cc1cc(C(=O)N2CCCN(c3ccc(N4CCCC4)nc3)CC2)cc(F)c1O] | 3-fluoro-2-hydroxy-5-(4-(6- (pyrrolidin-1-yl)pyridin-3-yl)- 1,4-diazepane-1- carbonyl)benzaldehyde trifluoroacetate salt |
| 142 | 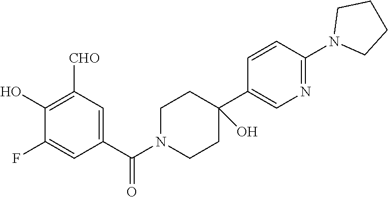 [SMILES: O=Cc1cc(C(=O)N2CCC(O)(c3ccc(N4CCCC4)nc3)CC2)cc(F)c1O] | 3-fluoro-2-hydroxy-5-(4- hydroxy-4-(6-(pyrrolidin-1- yl)pyridin-3-yl)piperidine-1- carbonyl)benzaldehyde |
| 143 | 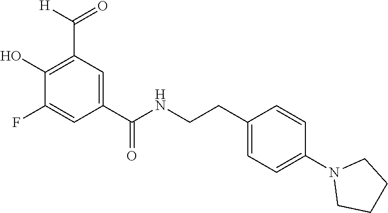 [SMILES: O=Cc1cc(C(=O)NCCc2ccc(N3CCCC3)cc2)cc(F)c1O] | 3-fluoro-5-formyl-4-hydroxy- N-(4-(pyrrolidin-1- yl)phenethyl)benzamide |
| 144 | 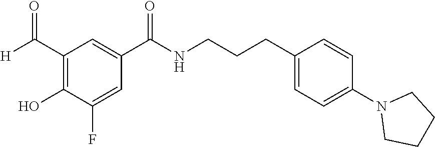 [SMILES: O=Cc1cc(C(=O)NCCCc2ccc(N3CCCC3)cc2)cc(F)c1O] | 3-fluoro-5-formyl-4-hydroxy- N-(3-(4-(pyrrolidin-1- yl)phenyl)propyl)benzamide |
| 145 | 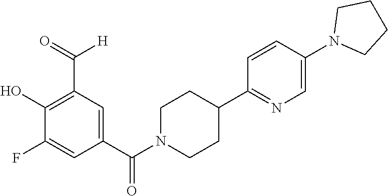 [SMILES: O=Cc1cc(C(=O)N2CCC(c3ccc(N4CCCC4)cn3)CC2)cc(F)c1O] | 3-fluoro-2-hydroxy-5-(4-(5- (pyrrolidin-1-yl)pyridin-2- yl)piperidine-1- carbonyl)benzaldehyde |
| 146 | 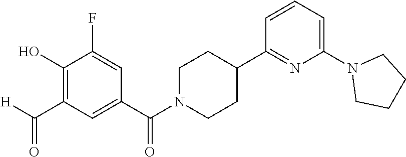 [SMILES: O=Cc1cc(C(=O)N2CCC(c3cccc(N4CCCC4)n3)CC2)cc(F)c1O] | 3-fluoro-2-hydroxy-5-(4-(6- (pyrrolidin-1-yl)pyridin-2- yl)piperidine-1- carbonyl)benzaldehyde |
| 147 | 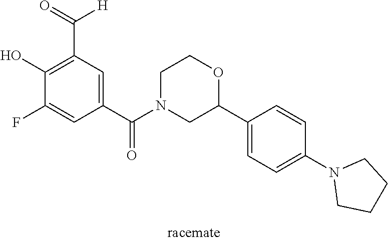 [SMILES: O=Cc1cc(C(=O)N2CCOC(c3ccc(N4CCCC4)cc3)C2)cc(F)c1O] | rac-3-fluoro-2-hydroxy-5-(2- (4-(pyrrolidin-1- yl)phenyl)morpholine-4- carbonyl)benzaldehyde |
| 148 | 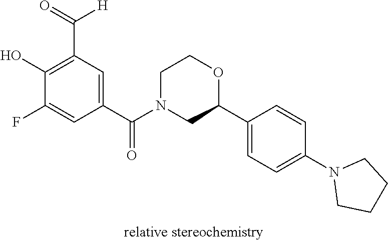 [SMILES: O=Cc1cc(C(=O)N2CCO[C@@H](c3ccc(N4CCCC4)cc3)C2)cc(F)c1O] | relative-(S)-3-fluoro-2- hydroxy-5-(2-(4-(pyrrolidin-1- yl)phenyl)morpholine-4- carbonyl)benzaldehyde |
| 149 | 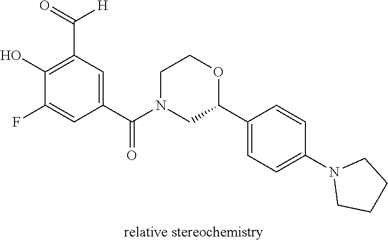 [SMILES: O=Cc1cc(C(=O)N2CCO[C@H](c3ccc(N4CCCC4)cc3)C2)cc(F)c1O] | relative-(R)-3-fluoro-2- hydroxy-5-(2-(4-(pyrrolidin-1- yl)phenyl)morpholine-4- carbonyl)benzaldehyde |
| 150 | 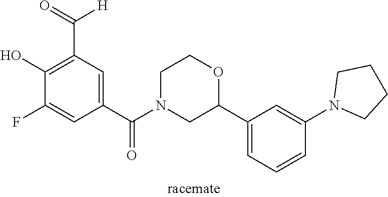 [SMILES: O=Cc1cc(C(=O)N2CCOC(c3cccc(N4CCCC4)c3)C2)cc(F)c1O] | rac-3-fluoro-2-hydroxy-5-(2- (3-(pyrrolidin-1- yl)phenyl)morpholine-4- carbonyl)benzaldehyde |
| 151 | 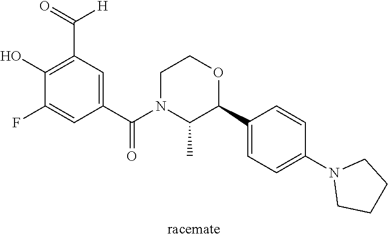 [SMILES: C[C@H]1[C@H](c2ccc(N3CCCC3)cc2)OCCN1C(=O)c1cc(F)c(O)c(C=O)c1] | rac-3-fluoro-2-hydroxy-5- ((2S,3S)-3-methyl-2-(4- (pyrrolidin-1- yl)phenyl)morpholine-4- carbonyl)benzaldehyde |
| 152 | 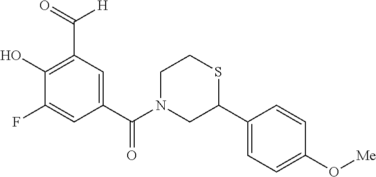 [SMILES: COc1ccc(C2CN(C(=O)c3cc(F)c(O)c(C=O)c3)CCS2)cc1] | rac-3-fluoro-2-hydroxy-5-(2- (4- methoxyphenyl)thiomorpholine- 4-carbonyl)benzaldehyde |
| 153 | 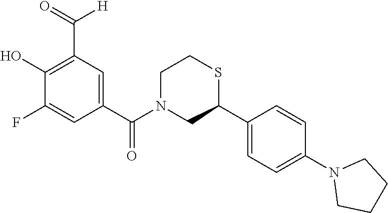 [SMILES: O=Cc1cc(C(=O)N2CCS[C@@H](c3ccc(N4CCCC4)cc3)C2)cc(F)c1O] | relative-(S)-3-fluoro-2- hydroxy-5-(2-(4-(pyrrolidin-1- yl)phenyl)thiomorpholine-4- carbonyl)benzaldehyde |
| 154 | 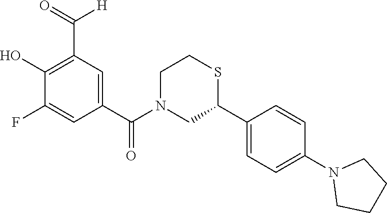 [SMILES: O=Cc1cc(C(=O)N2CCS[C@H](c3ccc(N4CCCC4)cc3)C2)cc(F)c1O] | relative-(R)-3-fluoro-2- hydroxy-5-(2-(4-(pyrrolidin-1- yl)phenyl)thiomorpholine-4- carbonyl)benzaldehyde |
| 155 | 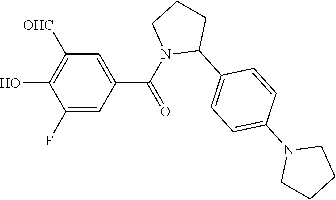 [SMILES: O=Cc1cc(C(=O)N2CCCC2c2ccc(N3CCCC3)cc2)cc(F)c1O] | 3-fluoro-2-hydroxy-5-(2-(4- (pyrrolidin-1- yl)phenyl)pyrrolidine-1- carbonyl)benzaldehyde |
| 156 | 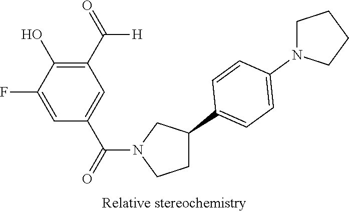 [SMILES: O=Cc1cc(C(=O)N2CC[C@H](c3ccc(N4CCCC4)cc3)C2)cc(F)c1O] | relative-(R)-3-fluoro-2- hydroxy-5-(3-(4-(pyrrolidin-1- yl)phenyl)pyrrolidine-1- carbonyl)benzaldehyde |
| 157 | 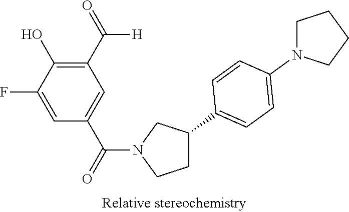 [SMILES: O=Cc1cc(C(=O)N2CC[C@@H](c3ccc(N4CCCC4)cc3)C2)cc(F)c1O] | relative-(S)-3-fluoro-2- hydroxy-5-(3-(4-(pyrrolidin-1- yl)phenyl)pyrrolidine-1- carbonyl)benzaldehyde |
| 158 | 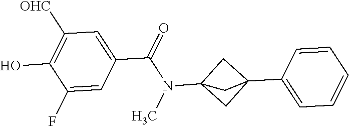 [SMILES: CN(C(=O)c1cc(F)c(O)c(C=O)c1)C12CC(c3ccccc3)(C1)C2] | 3-fluoro-5-formyl-4-hydroxy- N-methyl-N-(3- phenylbicyclo[1.1.1]pentan-1- yl)benzamide |
| 159 | 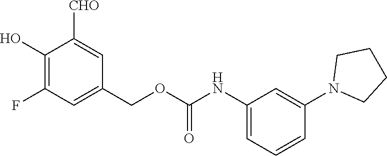 [SMILES: O=Cc1cc(COC(=O)Nc2cccc(N3CCCC3)c2)cc(F)c1O] | 3-fluoro-5-formyl-4- hydroxybenzyl (3-(pyrrolidin- 1-yl)phenyl)carbamate |
| 160 | 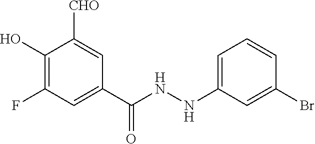 [SMILES: O=Cc1cc(C(=O)NNc2cccc(Br)c2)cc(F)c1O] | N′-(3-bromophenyl)-3-fluoro- 5-formyl-4- hydroxybenzohydrazide |
| 161 | 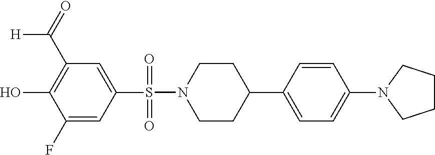 [SMILES: O=Cc1cc(S(=O)(=O)N2CCC(c3ccc(N4CCCC4)cc3)CC2)cc(F)c1O] | 3-fluoro-2-hydroxy-5-((4-(4- (pyrrolidin-1- yl)phenyl)piperidin-1- yl)sulfonyl)benzaldehyde |
| 162 | 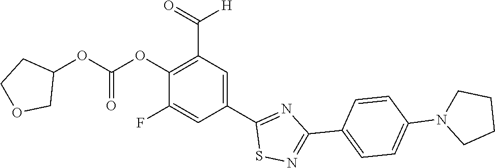 [SMILES: O=Cc1cc(-c2nc(-c3ccc(N4CCCC4)cc3)ns2)cc(F)c1OC(=O)OC1CCOC1] | 2-fluoro-6-formyl-4-(3-(4- (pyrrolidin-1-yl)phenyl)-1,2,4- thiadiazol-5-yl)phenyl (tetrahydrofuran-3-yl) carbonate |
| 163 | 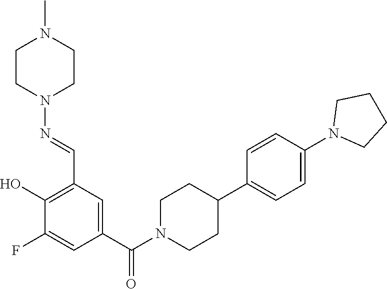 [SMILES: CN1CCN(/N=C/c2cc(C(=O)N3CCC(c4ccc(N5CCCC5)cc4)CC3)cc(F)c2O)CC1] | (3-fluoro-4-hydroxy-5-(((4- methylpiperazin-1- yl)imino)methyl)phenyl)(4-(4- (pyrrolidin-1- yl)phenyl)piperidin-1- yl)methanone |
| 164 | 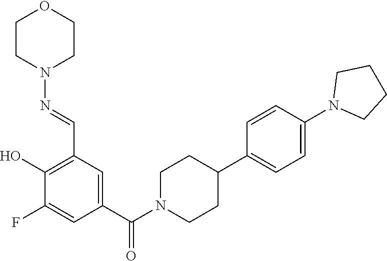 [SMILES: O=C(c1cc(F)c(O)c(/C=N/N2CCOCC2)c1)N1CCC(c2ccc(N3CCCC3)cc2)CC1] | (3-fluoro-4-hydroxy-5- ((morpholinoimino)methyl) phenyl)(4-(4-(pyrrolidin-1- yl)phenyl)piperidin-1- yl)methanone |
| 165 | 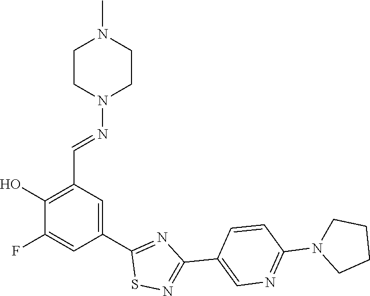 [SMILES: CN1CCN(/N=C/c2cc(-c3nc(-c4ccc(N5CCCC5)nc4)ns3)cc(F)c2O)CC1] | (E)-2-fluoro-6-(((4- methylpiperazin-1- yl)imino)methyl)-4-(3-(6- (pyrrolidin-1-yl)pyridin-3-yl)- 1,2,4-thiadiazol-5-yl)phenol |
| 166 | 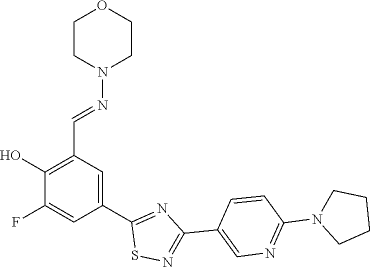 [SMILES: Oc1c(F)cc(-c2nc(-c3ccc(N4CCCC4)nc3)ns2)cc1/C=N/N1CCOCC1] | 2-fluoro-6- ((morpholinoimino)methyl)-4- (3-(4-(pyrrolidin-1-yl)phenyl)- 1,2,4-thiadiazol-5-yl)phenol |
| 167 | 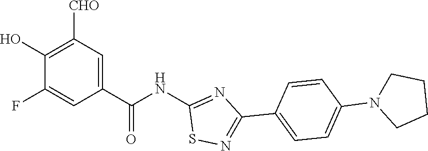 [SMILES: O=Cc1cc(C(=O)Nc2nc(-c3ccc(N4CCCC4)cc3)ns2)cc(F)c1O] | 3-fluoro-5-formyl-4-hydroxy- N-(3-(4-(pyrrolidin-1- yl)phenyl)-1,2,4-thiadiazol-5- yl)benzamide |
| 168 | 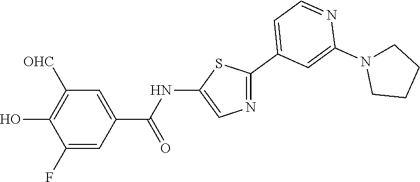 [SMILES: O=Cc1cc(C(=O)Nc2cnc(-c3ccnc(N4CCCC4)c3)s2)cc(F)c1O] | 3-fluoro-5-formyl-4-hydroxy- N-(2-(2-(pyrrolidin-1- yl)pyridin-4-yl)thiazol-5- yl)benzamide |
| 169 | 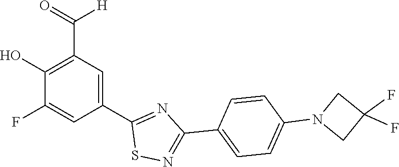 [SMILES: O=Cc1cc(-c2nc(-c3ccc(N4CC(F)(F)C4)cc3)ns2)cc(F)c1O] | 5-(3-(4-(3,3-difluoroazetidin- 1-yl)phenyl)-1,2,4-thiadiazol- 5-yl)-3-fluoro-2- hydroxybenzaldehyde |
| 170 | 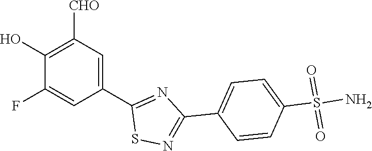 [SMILES: NS(=O)(=O)c1ccc(-c2nsc(-c3cc(F)c(O)c(C=O)c3)n2)cc1] | 4-(5-(3-fluoro-5-formyl-4- hydroxyphenyl)-1,2,4- thiadiazol-3- yl)benzenesulfonamide |
| 171 | 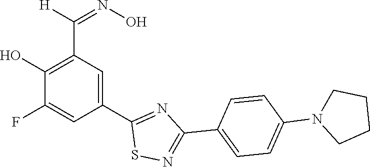 [SMILES: O/N=C\c1cc(-c2nc(-c3ccc(N4CCCC4)cc3)ns2)cc(F)c1O] | (Z)-3-fluoro-2-hydroxy-5-(3- (4-(pyrrolidin-1-yl)phenyl)- 1,2,4-thiadiazol-5- yl)benzaldehyde oxime |
| 172 | 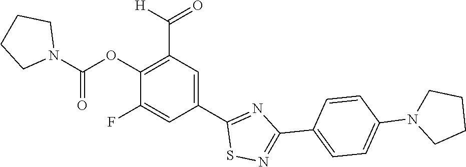 [SMILES: O=Cc1cc(-c2nc(-c3ccc(N4CCCC4)cc3)ns2)cc(F)c1OC(=O)N1CCCC1] | 2-fluoro-6-formyl-4-(3-(4- (pyrrolidin-1-yl)phenyl)-1,2,4- thiadiazol-5-yl)phenyl pyrrolidine-1-carboxylate |
| 173 | 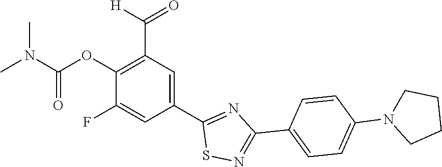 [SMILES: CN(C)C(=O)Oc1c(F)cc(-c2nc(-c3ccc(N4CCCC4)cc3)ns2)cc1C=O] | 2-fluoro-6-formyl-4-(3-(4- (pyrrolidin-1-yl)phenyl)-1,2,4- thiadiazol-5-yl)phenyl dimethylcarbamate |
| 174 | 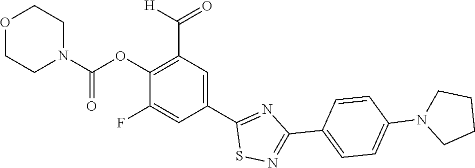 [SMILES: O=Cc1cc(-c2nc(-c3ccc(N4CCCC4)cc3)ns2)cc(F)c1OC(=O)N1CCOCC1] | 2-fluoro-6-formyl-4-(3-(4- (pyrrolidin-1-yl)phenyl)-1,2,4- thiadiazol-5-yl)phenyl morpholine-4-carboxylate |
| 175 | 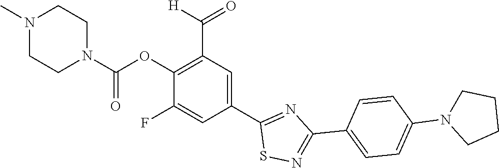 [SMILES: CN1CCN(C(=O)Oc2c(F)cc(-c3nc(-c4ccc(N5CCCC5)cc4)ns3)cc2C=O)CC1] | 2-fluoro-6-formyl-4-(3-(4- (pyrrolidin-1-yl)phenyl)-1,2,4- thiadiazol-5-yl)phenyl 4- methylpiperazine-1- carboxylate |
| 176 | 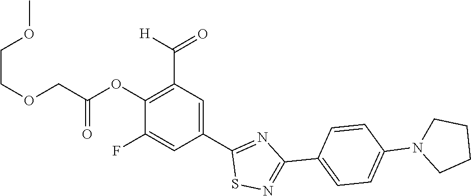 [SMILES: COCCOCC(=O)Oc1c(F)cc(-c2nc(-c3ccc(N4CCCC4)cc3)ns2)cc1C=O] | 2-fluoro-6-formyl-4-(3-(4- (pyrrolidin-1-yl)phenyl)-1,2,4- thiadiazol-5-yl)phenyl 2-(2- methoxyethoxy)acetate |
| 177 | 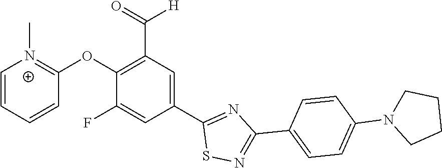 [SMILES: C[n+]1ccccc1Oc1c(F)cc(-c2nc(-c3ccc(N4CCCC4)cc3)ns2)cc1C=O] | 2-(2-fluoro-6-formyl-4-(3-(4- (pyrrolidin-1-yl)phenyl)-1,2,4- thiadiazol-5-yl)phenoxy)-1- methylpyridin-1-ium |
| 178 | 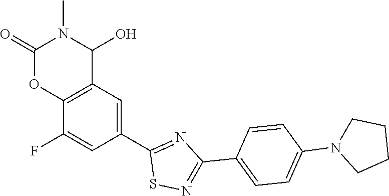 [SMILES: CN1C(=O)Oc2c(F)cc(-c3nc(-c4ccc(N5CCCC5)cc4)ns3)cc2C1O] | 8-fluoro-4-hydroxy-3-methyl- 6-(3-(4-(pyrrolidin-1- yl)phenyl)-1,2,4-thiadiazol-5- yl)-3,4-dihydro-2H- benzo[e][1,3]oxazin-2-one |
| 179 | 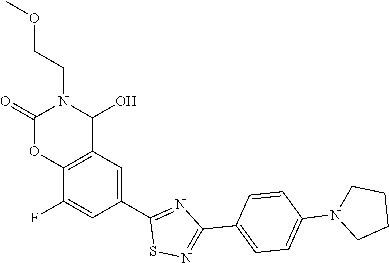 [SMILES: COCCN1C(=O)Oc2c(F)cc(-c3nc(-c4ccc(N5CCCC5)cc4)ns3)cc2C1O] | 8-fluoro-4-hydroxy-3-(2- methoxyethyl)-6-(3-(4- (pyrrolidin-1-yl)phenyl)-1,2,4- thiadiazol-5-yl)-3,4-dihydro- 2H-benzo[e][1,3]oxazin-2-one |
| 180 | 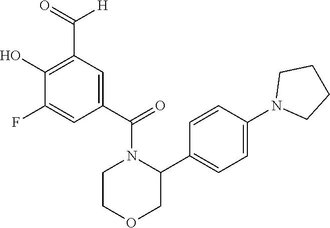 [SMILES: O=Cc1cc(C(=O)N2CCOCC2c2ccc(N3CCCC3)cc2)cc(F)c1O] | 3-fluoro-2-hydroxy-5-(3-(4- (pyrrolidin-1- yl)phenyl)morpholine-4- carbonyl)benzaldehyde |
| 181 | 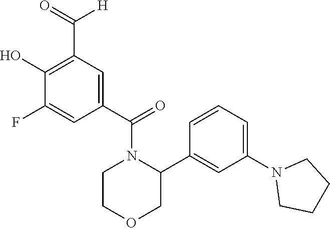 [SMILES: O=Cc1cc(C(=O)N2CCOCC2c2cccc(N3CCCC3)c2)cc(F)c1O] | 3-fluoro-2-hydroxy-5-(3-(3- (pyrrolidin-1- yl)phenyl)morpholine-4- carbonyl)benzaldehyde |
| 182 | 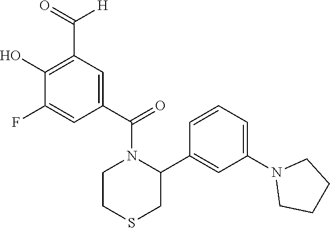 [SMILES: O=Cc1cc(C(=O)N2CCSCC2c2cccc(N3CCCC3)c2)cc(F)c1O] | 3-fluoro-2-hydroxy-5-(3-(3- (pyrrolidin-1- yl)phenyl)thiomorpholine-4- carbonyl)benzaldehyde |
| 183 | 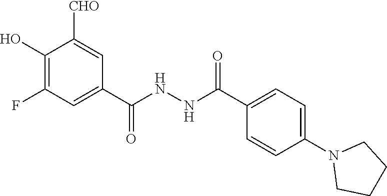 [SMILES: O=Cc1cc(C(=O)NNC(=O)c2ccc(N3CCCC3)cc2)cc(F)c1O] | 3-fluoro-5-formyl-4-hydroxy- N′-(4-(pyrrolidin-1- yl)benzoyl)benzohydrazide |
| 184 | 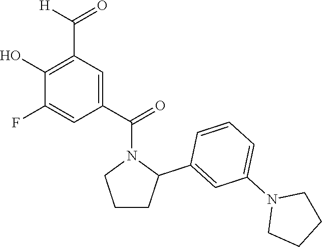 [SMILES: O=Cc1cc(C(=O)N2CCCC2c2cccc(N3CCCC3)c2)cc(F)c1O] | 3-fluoro-2-hydroxy-5-(2-(3- (pyrrolidin-1- yl)phenyl)pyrrolidine-1- carbonyl)benzaldehyde |
| 185 | 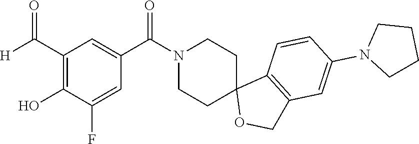 [SMILES: O=Cc1cc(C(=O)N2CCC3(CC2)OCc2cc(N4CCCC4)ccc23)cc(F)c1O] | 3-fluoro-2-hydroxy-5-(5- (pyrrolidin-1-yl)-3H- spiro[isobenzofuran-1,4′- piperidine]-1′- carbonyl)benzaldehyde |
| 186 | 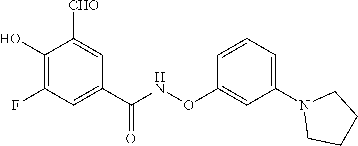 [SMILES: O=Cc1cc(C(=O)NOc2cccc(N3CCCC3)c2)cc(F)c1O] | 3-fluoro-5-formyl-4-hydroxy- N-(3-(pyrrolidin-1- yl)phenoxy)benzamide |
| 187 | 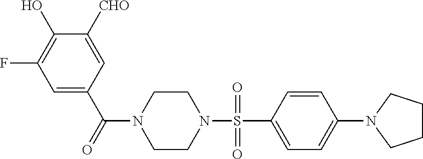 [SMILES: O=Cc1cc(C(=O)N2CCN(S(=O)(=O)c3ccc(N4CCCC4)cc3)CC2)cc(F)c1O] | 3-fluoro-2-hydroxy-5-(4-((4- (pyrrolidin-1- yl)phenyl)sulfonyl)piperazine- 1-carbonyl)benzaldehyde |
| 188 | 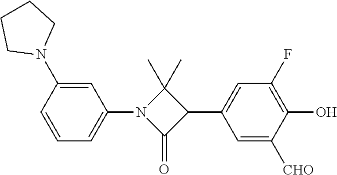 [SMILES: CC1(C)C(c2cc(F)c(O)c(C=O)c2)C(=O)N1c1cccc(N2CCCC2)c1] | 5-(2,2-dimethyl-4-oxo-1-(3- (pyrrolidin-1- yl)phenyl)azetidin-3-yl)-3- fluoro-2-hydroxybenzaldehyde |
| 189 | 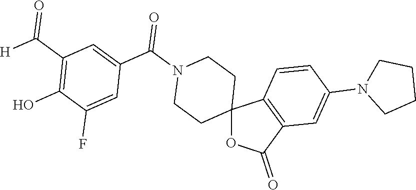 [SMILES: O=Cc1cc(C(=O)N2CCC3(CC2)OC(=O)c2cc(N4CCCC4)ccc23)cc(F)c1O] | 3-fluoro-2-hydroxy-5-(3-oxo- 5-(pyrrolidin-1-yl)-3H- spiro[isobenzofuran-1,4′- piperidine]-1′- carbonyl)benzaldehyde |
| 190 | 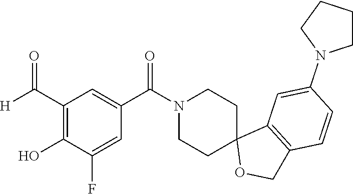 [SMILES: O=Cc1cc(C(=O)N2CCC3(CC2)OCc2ccc(N4CCCC4)cc23)cc(F)c1O] | 3-fluoro-2-hydroxy-5-(6- (pyrrolidin-1-yl)-3H- spiro[isobenzofuran-1,4′- piperidine]-1′- carbonyl)benzaldehyde |
| 191 | 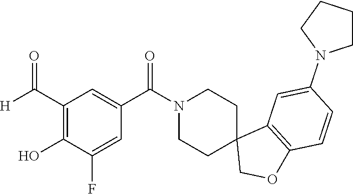 [SMILES: O=Cc1cc(C(=O)N2CCC3(CC2)COc2ccc(N4CCCC4)cc23)cc(F)c1O] | 3-fluoro-2-hydroxy-5-(5- (pyrrolidin-1-yl)-2H- spiro[benzofuran-3,4′- piperidine]-1′- carbonyl)benzaldehyde |
| 192 | 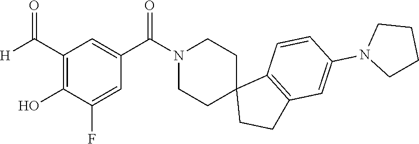 [SMILES: O=Cc1cc(C(=O)N2CCC3(CCc4cc(N5CCCC5)ccc43)CC2)cc(F)c1O] | 3-fluoro-2-hydroxy-5-(5- (pyrrolidin-1-yl)-2,3- dihydrospiro[indene-1,4′- piperidine]-1′- carbonyl)benzaldehyde |
| 193 | 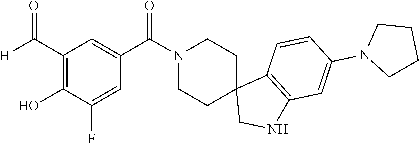 [SMILES: O=Cc1cc(C(=O)N2CCC3(CC2)CNc2cc(N4CCCC4)ccc23)cc(F)c1O] | 3-fluoro-2-hydroxy-5-(6- (pyrrolidin-1- yl)spiro[indoline-3,4′- piperidine]-1′- carbonyl)benzaldehyde |
| 194 | 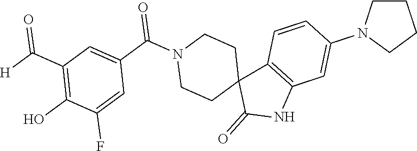 [SMILES: O=Cc1cc(C(=O)N2CCC3(CC2)C(=O)Nc2cc(N4CCCC4)ccc23)cc(F)c1O] | 3-fluoro-2-hydroxy-5-(2-oxo- 6-(pyrrolidin-1- yl)spiro[indoline-3,4′- piperidine]-1′- carbonyl)benzaldehyde |
| 195 | 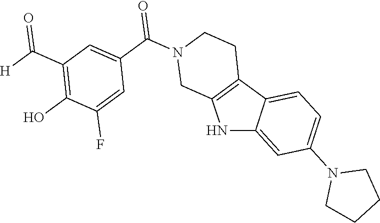 [SMILES: O=Cc1cc(C(=O)N2CCc3c([nH]c4cc(N5CCCC5)ccc34)C2)cc(F)c1O] | 3-fluoro-2-hydroxy-5-(7- (pyrrolidin-1-yl)-2,3,4,9- tetrahydro-1H-pyrido[3,4- b]indole-2- carbonyl)benzaldehyde |
| 196 | 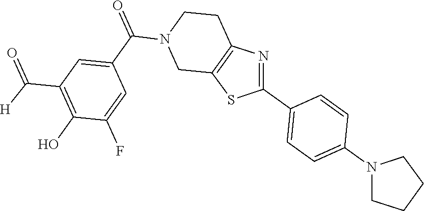 [SMILES: O=Cc1cc(C(=O)N2CCc3nc(-c4ccc(N5CCCC5)cc4)sc3C2)cc(F)c1O] | 3-fluoro-2-hydroxy-5-(2-(4- (pyrrolidin-1-yl)phenyl)- 4,5,6,7-tetrahydrothiazolo[5,4- c]pyridine-5- carbonyl)benzaldehyde |
| 197 | 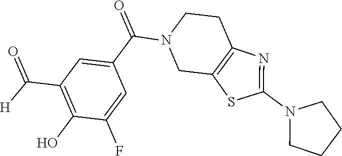 [SMILES: O=Cc1cc(C(=O)N2CCc3nc(N4CCCC4)sc3C2)cc(F)c1O] | 3-fluoro-2-hydroxy-5-(2- (pyrrolidin-1-yl)-4,5,6,7- tetrahydrothiazolo[5,4- c]pyridine-5- carbonyl)benzaldehyde |
| 198 | 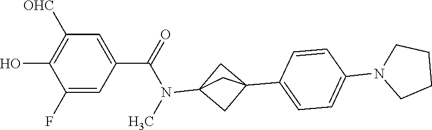 [SMILES: CN(C(=O)c1cc(F)c(O)c(C=O)c1)C12CC(c3ccc(N4CCCC4)cc3)(C1)C2] | 3-fluoro-5-formyl-4-hydroxy- N-methyl-N-(3-(4-(pyrrolidin- 1- yl)phenyl)bicyclo[1.1.1]pentan- 1-yl)benzamide |
| 199 | 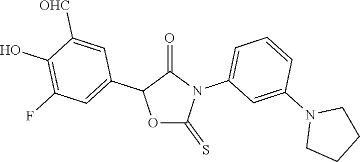 [SMILES: O=Cc1cc(C2OC(=S)N(c3cccc(N4CCCC4)c3)C2=O)cc(F)c1O] | 3-fluoro-2-hydroxy-5-(4-oxo- 3-(3-(pyrrolidin-1-yl)phenyl)- 2-thioxooxazolidin-5- yl)benzaldehyde |
| 200 | 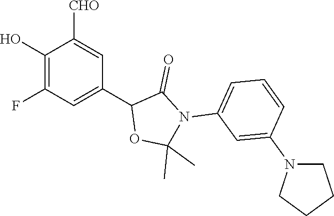 [SMILES: CC1(C)OC(c2cc(F)c(O)c(C=O)c2)C(=O)N1c1cccc(N2CCCC2)c1] | 5-(2,2-dimethyl-4-oxo-3-(3- (pyrrolidin-1- yl)phenyl)oxazolidin-5-yl)-3- fluoro-2-hydroxybenzaldehyde |
| 201 | 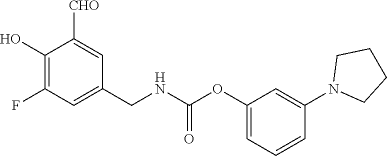 [SMILES: O=Cc1cc(CNC(=O)Oc2cccc(N3CCCC3)c2)cc(F)c1O] | 3-(pyrrolidin-1-yl)phenyl (3- fluoro-5-formyl-4- hydroxybenzyl)carbamate |
| 202 | 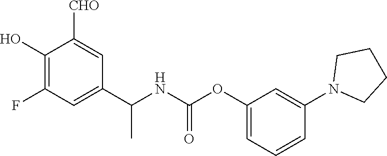 [SMILES: CC(NC(=O)Oc1cccc(N2CCCC2)c1)c1cc(F)c(O)c(C=O)c1] | 3-(pyrrolidin-1-yl)phenyl (1- (3-fluoro-5-formyl-4- hydroxyphenyl)ethyl) carbamate |
| 203 | 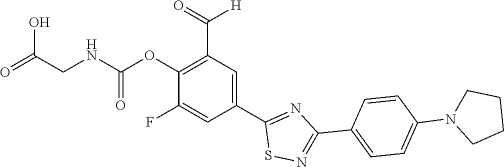 [SMILES: O=Cc1cc(-c2nc(-c3ccc(N4CCCC4)cc3)ns2)cc(F)c1OC(=O)NCC(=O)O] | ((2-fluoro-6-formyl-4-(3-(4- (pyrrolidin-1-yl)phenyl)-1,2,4- thiadiazol-5- yl)phenoxy)carbonyl)glycine |
| 204 | 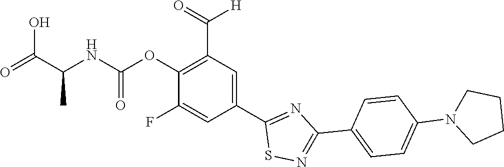 [SMILES: C[C@H](NC(=O)Oc1c(F)cc(-c2nc(-c3ccc(N4CCCC4)cc3)ns2)cc1C=O)C(=O)O] | ((2-fluoro-6-formyl-4-(3-(4- (pyrrolidin-1-yl)phenyl)-1,2,4- thiadiazol-5- yl)phenoxy)carbonyl)-L- alanine |
| 205 | 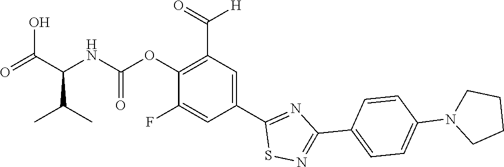 [SMILES: CC(C)[C@H](NC(=O)Oc1c(F)cc(-c2nc(-c3ccc(N4CCCC4)cc3)ns2)cc1C=O)C(=O)O] | ((2-fluoro-6-formyl-4-(3-(4- (pyrrolidin-1-yl)phenyl)-1,2,4- thiadiazol-5- yl)phenoxy)carbonyl)-L-valine |
| 206 | 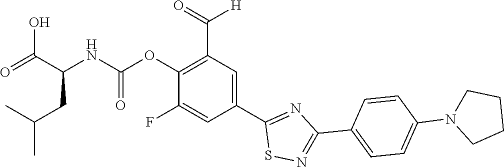 [SMILES: CC(C)C[C@H](NC(=O)Oc1c(F)cc(-c2nc(-c3ccc(N4CCCC4)cc3)ns2)cc1C=O)C(=O)O] | ((2-fluoro-6-formyl-4-(3-(4- (pyrrolidin-1-yl)phenyl)-1,2,4- thiadiazol-5- yl)phenoxy)carbonyl)-L- leucine |
| 207 | 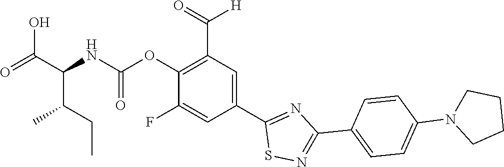 [SMILES: CC[C@H](C)[C@H](NC(=O)Oc1c(F)cc(-c2nc(-c3ccc(N4CCCC4)cc3)ns2)cc1C=O)C(=O)O] | ((2-fluoro-6-formyl-4-(3-(4- (pyrrolidin-1-yl)phenyl)-1,2,4- thiadiazol-5- yl)phenoxy)carbonyl)-L- isoleucine |
| 208 | 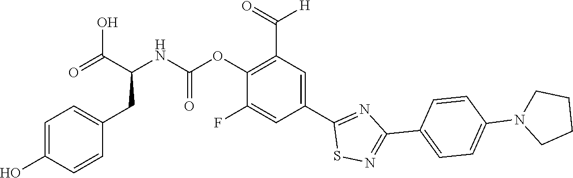 [SMILES: O=Cc1cc(-c2nc(-c3ccc(N4CCCC4)cc3)ns2)cc(F)c1OC(=O)N[C@@H](Cc1ccc(O)cc1)C(=O)O] | ((2-fluoro-6-formyl-4-(3-(4- (pyrrolidin-1-yl)phenyl)-1,2,4- thiadiazol-5- yl)phenoxy)carbonyl)-L- tyrosine |
| 209 | 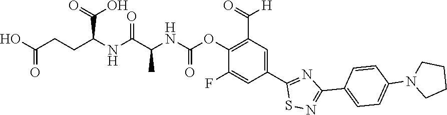 [SMILES: C[C@H](NC(=O)Oc1c(F)cc(-c2nc(-c3ccc(N4CCCC4)cc3)ns2)cc1C=O)C(=O)N[C@@H](CCC(=O)O)C(=O)O] | ((2-fluoro-6-formyl-4-(3-(4- (pyrrolidin-1-yl)phenyl)-1,2,4- thiadiazol-5- yl)phenoxy)carbonyl)-L- tyrosine |
| 210 | 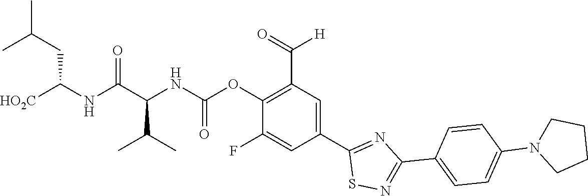 [SMILES: CC(C)C[C@H](NC(=O)[C@@H](NC(=O)Oc1c(F)cc(-c2nc(-c3ccc(N4CCCC4)cc3)ns2)cc1C=O)C(C)C)C(=O)O] | ((2-fluoro-6-formyl-4-(3-(4- (pyrrolidin-1-yl)phenyl)-1,2,4- thiadiazol-5- yl)phenoxy)carbonyl)-L-valyl- L-leucine |
| 211 | 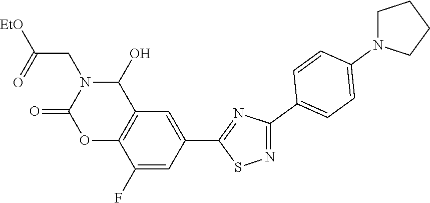 [SMILES: CCOC(=O)CN1C(=O)Oc2c(F)cc(-c3nc(-c4ccc(N5CCCC5)cc4)ns3)cc2C1O] | ethyl 2-(8-fluoro-4-hydroxy-2- oxo-6-(3-(4-(pyrrolidin-1- yl)phenyl)-1,2,4-thiadiazol-5- yl)-2H-benzo[e][1,3]oxazin- 3(4H)-yl)acetate |
| 212 | 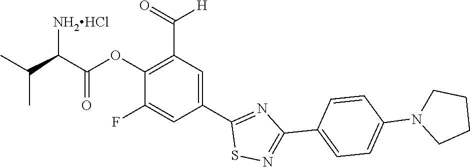 | 2-fluoro-6-formyl-4-(3-(4- (pyrrolidin-1-yl)phenyl)-1,2,4- thiadiazol-5-yl)phenyl D- valinate hydrochloride |
| 213 | 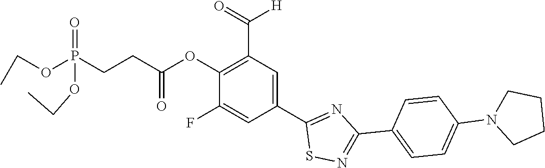 [SMILES: CCOP(=O)(CCC(=O)Oc1c(F)cc(-c2nc(-c3ccc(N4CCCC4)cc3)ns2)cc1C=O)OCC] | 2-fluoro-6-formyl-4-(3-(4- (pyrrolidin-1-yl)phenyl)-1,2,4- thiadiazol-5-yl)phenyl 3- (diethoxyphosphoryl) propanoate |
| 214 | 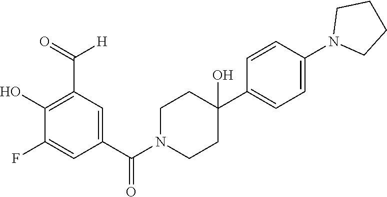 [SMILES: O=Cc1cc(C(=O)N2CCC(O)(c3ccc(N4CCCC4)cc3)CC2)cc(F)c1O] | 3-fluoro-2-hydroxy-5-(4- hydroxy-4-(4-(pyrrolidin-1- yl)phenyl)piperidine-1- carbonyl)benzaldehyde |
| 215 | 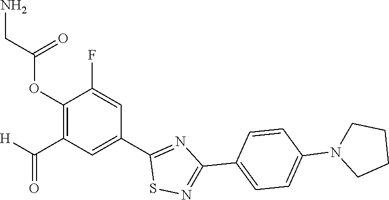 [SMILES: NCC(=O)Oc1c(F)cc(-c2nc(-c3ccc(N4CCCC4)cc3)ns2)cc1C=O] | 2-fluoro-6-formyl-4-(3-(4- (pyrrolidin-1-yl)phenyl)-1,2,4- thiadiazol-5-yl)phenyl glycinate |
| 216 | 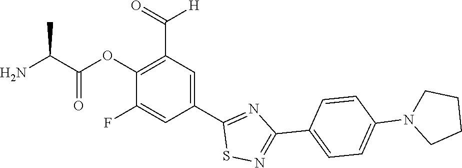 [SMILES: C[C@H](N)C(=O)Oc1c(F)cc(-c2nc(-c3ccc(N4CCCC4)cc3)ns2)cc1C=O] | 2-fluoro-6-formyl-4-(3-(4- (pyrrolidin-1-yl)phenyl)-1,2,4- thiadiazol-5-yl)phenyl L- alaninate |
| 217 | 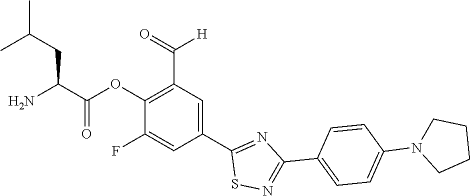 [SMILES: CC(C)C[C@H](N)C(=O)Oc1c(F)cc(-c2nc(-c3ccc(N4CCCC4)cc3)ns2)cc1C=O] | 2-fluoro-6-formyl-4-(3-(4- (pyrrolidin-1-yl)phenyl)-1,2,4- thiadiazol-5-yl)phenyl L- leucinate |
| 218 | 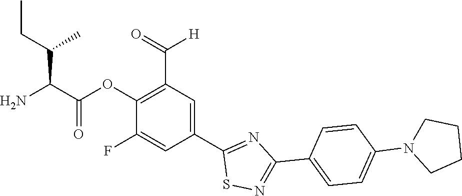 [SMILES: CC[C@H](C)[C@H](N)C(=O)Oc1c(F)cc(-c2nc(-c3ccc(N4CCCC4)cc3)ns2)cc1C=O] | 2-fluoro-6-formyl-4-(3-(4- (pyrrolidin-1-yl)phenyl)-1,2,4- thiadiazol-5-yl)phenyl (2S,3S)-2-(12-azanyl)-3- methylpentanoate |
| 219 | 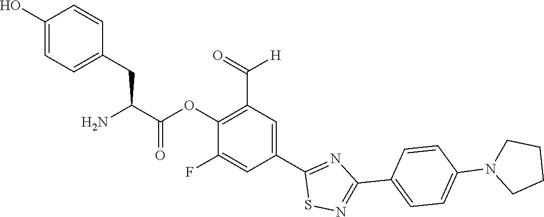 [SMILES: N[C@@H](Cc1ccc(O)cc1)C(=O)Oc1c(F)cc(-c2nc(-c3ccc(N4CCCC4)cc3)ns2)cc1C=O] | 2-fluoro-6-formyl-4-(3-(4- (pyrrolidin-1-yl)phenyl)-1,2,4- thiadiazol-5-yl)phenyl (S)-2- (12-azanyl)-3-(4- hydroxyphenyl)propanoate |
| 220 | 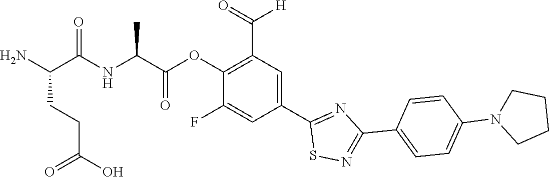 [SMILES: C[C@H](NC(=O)[C@@H](N)CCC(=O)O)C(=O)Oc1c(F)cc(-c2nc(-c3ccc(N4CCCC4)cc3)ns2)cc1C=O] | (S)-4-amino-5-(((S)-1-(2- fluoro-6-formyl-4-(3-(4- (pyrrolidin-1-yl)phenyl)-1,2,4- thiadiazol-5-yl)phenoxy)-1- oxopropan-2-yl)amino)-5- oxopentanoic acid |
| 221 | 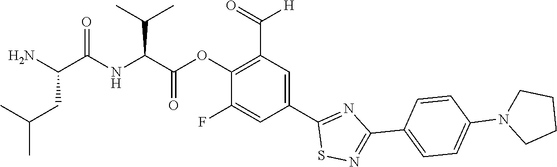 [SMILES: CC(C)C[C@H](N)C(=O)N[C@H](C(=O)Oc1c(F)cc(-c2nc(-c3ccc(N4CCCC4)cc3)ns2)cc1C=O)C(C)C] | 2-fluoro-6-formyl-4-(3-(4- (pyrrolidin-1-yl)phenyl)-1,2,4- thiadiazol-5-yl)phenyl L- leucyl-L-valinate |
| 222 | 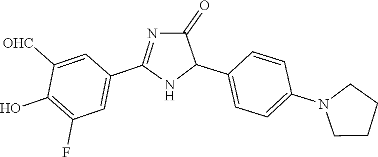 [SMILES: O=Cc1cc(C2=NC(=O)C(c3ccc(N4CCCC4)cc3)N2)cc(F)c1O] | 3-fluoro-2-hydroxy-5-(4-oxo- 5-(4-(pyrrolidin-1-yl)phenyl)- 4,5-dihydro-1H-imidazol-2- yl)benzaldehyde |
| 223 | 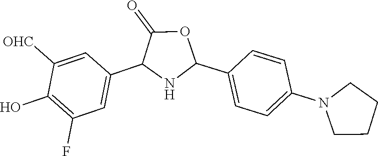 [SMILES: O=Cc1cc(C2NC(c3ccc(N4CCCC4)cc3)OC2=O)cc(F)c1O] | 3-fluoro-2-hydroxy-5-(5-oxo- 2-(4-(pyrrolidin-1- yl)phenyl)oxazolidin-4- yl)benzaldehyde |
| 224 | 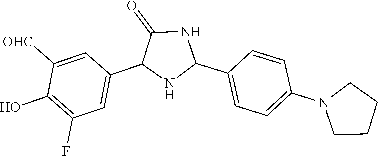 [SMILES: O=Cc1cc(C2NC(c3ccc(N4CCCC4)cc3)NC2=O)cc(F)c1O] | 3-fluoro-2-hydroxy-5-(5-oxo- 2-(4-(pyrrolidin-1- yl)phenyl)imidazolidin-4- yl)benzaldehyde |
| 225 | 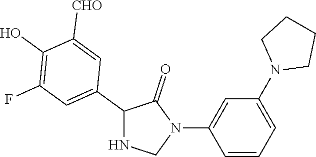 [SMILES: O=Cc1cc(C2NCN(c3cccc(N4CCCC4)c3)C2=O)cc(F)c1O] | 3-fluoro-2-hydroxy-5-(5-oxo- 1-(3-(pyrrolidin-1- yl)phenyl)imidazolidin-4- yl)benzaldehyde |
| 226 | 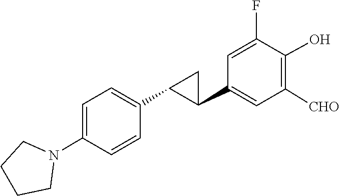 [SMILES: O=Cc1cc([C@@H]2C[C@H]2c2ccc(N3CCCC3)cc2)cc(F)c1O] | 3-fluoro-2-hydroxy-5- ((1R,2R)-2-(4-(pyrrolidin-1- yl)phenyl)cyclopropyl) benzaldehyde |
| 227 | 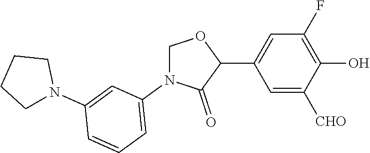 [SMILES: O=Cc1cc(C2OCN(c3cccc(N4CCCC4)c3)C2=O)cc(F)c1O] | 3-fluoro-2-hydroxy-5-(4-oxo- 3-(3-(pyrrolidin-1- yl)phenyl)oxazolidin-5- yl)benzaldehyde |
| 228 | 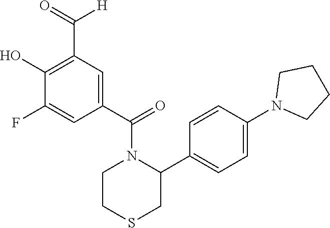 [SMILES: O=Cc1cc(C(=O)N2CCSCC2c2ccc(N3CCCC3)cc2)cc(F)c1O] | 3-fluoro-2-hydroxy-5-(3-(4- (pyrrolidin-1- yl)phenyl)thiomorpholine-4- carbonyl)benzaldehyde |

Any formula or compound given herein, such as Formula (I) or compounds of Table 1 is intended to represent compounds having structures depicted by the structural formula as well as certain variations or forms. In particular, compounds of any formula given herein may contain bonds with restricted rotation and therefore exist in different geometric confirgurations. Additionally, compounds of any formula provided herein may have asymmetric centers and therefore exist in different enantiomeric or diastereomeric forms. All optical isomers and stereoisomers of the compounds of the general formula, and mixtures thereof in any ratio, are considered within the scope of the formula. Thus, any formula given herein is intended to represent a racemate, one or more enantiomeric forms, one or more diastereomeric forms, one or more atropisomeric forms (e.g., geoisomeric forms), and mixtures thereof in any ratio. Where a compound of Table 1 is depicted with a particular stereochemical configuration, also provided herein is any alternative stereochemical configuration of the compound, as well as a mixture of stereoisomers of the compound in any ratio. Any compound of Table 1 is intended to represent a racemate, one or more enantiomeric forms, one or more diastereomeric forms, one or more atropisomeric forms (e.g., geoisomeric forms), and mixtures thereof in any ratio. Furthermore, certain structures may exist as tautomers or as atropisomers. Additionally, any formula given herein is intended to refer to hydrates, solvates, and amorphous forms of such compounds, and mixtures thereof, even if such forms are not listed explicitly. In some embodiments, the solvent is water and the solvates are hydrates.

The compounds of Formula (I), or Table 1 may be prepared and/or formulated as pharmaceutically acceptable salts. In some embodiments, pharmaceutically acceptable salts include acid addition salts, formed with inorganic acids such as hydrochloric acid, hydrobromic acid, sulfuric acid, nitric acid, phosphoric acid, and the like; or formed with organic acids such as acetic acid, oxalic acid, propionic acid, succinic acid, maleic acid, tartaric acid and the like. These salts may be derived from inorganic or organic acids. Non-limiting examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, 7-hydroxybutyrates, glycolates, tartrates, and mandelates. In some embodiments, pharmaceutically acceptable salts are formed when an acidic proton present in the parent compound either is replaced by a metal ion, e.g., an alkali metal ion, an alkaline earth ion, or an aluminum ion; or coordinates with an organic base. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, tromethamine, trimetharnine, dicyclohexylamine, caffeine, procaine, hydrabamine, choline, betaine, ethylenediamine, glucosamine, N-ethylglucamine, N-methylglucamine, theobromine, purines, piperazine, piperidine, N-ethylpiperidine, polyamine resins, amino acids such as lysine, arginine, histidine, and the like. Examples of pharmaceutically acceptable base addition salts include those derived from inorganic bases such as sodium, potassium, lithium, ammonium, calcium, magnesium, iron, zinc, copper, manganese, aluminum salts and the like. In some embodiments, the organic non-toxic bases are L-amino acids, such as L-lysine and L-arginine, tromethamine, N-ethylglucamine and N-methylglucamine. Acceptable inorganic bases include aluminum hydroxide, calcium hydroxide, potassium hydroxide, sodium carbonate, sodium hydroxide, and the like. Lists of other suitable pharmaceutically acceptable salts are found in Remington's Pharmaceutical Sciences, 17th Edition, Mack Publishing Company, Easton, Pa., 1985.

For a compound described herein that contains a basic nitrogen, a pharmaceutically acceptable salt may be prepared by any suitable method available in the art, for example, treatment of the free base with an inorganic acid, such as hydrochloric acid, hydrobromic acid, sulfuric acid, sulfamic acid, nitric acid, boric acid, phosphoric acid, and the like, or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid or glutamic acid, an aromatic acid, such as benzoic acid, 2-acetoxybenzoic acid, naphthoic acid, or cinnamic acid, a sulfonic acid, such as laurylsulfonic acid, p-toluenesulfonic acid, methanesulfonic acid, benzenesulfonic acid, or ethanesulfonic acid, or any compatible mixture of acids such as those given as examples herein, and any other acid and mixture thereof that are regarded as equivalents or acceptable substitutes in light of the ordinary level of skill in this technology.

The compounds depicted herein may be present as salts even if salts are not depicted, and it is understood that the compositions and methods provided herein embrace all salts and solvates of the compounds depicted here, as well as the non-salt and non-solvate form of the compound, as is well understood by the skilled artisan. In some embodiments, the salts of the compounds provided herein are pharmaceutically acceptable salts.

Representative examples of compounds detailed herein, including intermediates and final compounds, are depicted in the tables and elsewhere herein. It is understood that in one aspect, any of the compounds may be used in the methods detailed herein, including, where applicable, intermediate compounds that may be isolated and administered to an individual.

In one variation, the compounds herein are synthetic compounds prepared for administration to an individual. In another variation, compositions are provided containing a compound in substantially pure form. In another variation, provided are pharmaceutical compositions comprising a compound detailed herein and a pharmaceutically acceptable carrier. In another variation, methods of administering a compound are provided. The purified forms, pharmaceutical compositions and methods of administering the compounds are suitable for any compound or form thereof detailed herein.

Any variation or embodiment of R¹, R^{1A}, R², R^{2A}, R^{1A1}, R^{1A2}, R^{1A3}, m, n, R^{2A1}, R^{2A1A}, R^{2A1B}, R^{2A1C}, R^{2A1D}, R³, G¹, G², L, R^{4A}, R^{4B}, R^{4C}, R^{4D}, R^{4E}, R^{4F}, A¹, A², R^{1a1}, R^{1a2}, Y¹, Y², n¹, n², X, R^{A}, R^{A2}, R^{A3}, R^{A4}, R^{A5}, R^{A6}, and R^{A7} provided herein can be combined with every other variation or embodiment of R, R^{1A}, R², R^{2A}, R^{1A1}, R^{1A2}, R^{1A3}, m, n, R^{2A1}, R^{2A1A}, R^{2A1B}, R^{2A1C}, R^{2A1D}, R³, G¹, G², L, R^{4A}, R^{4B}, R^{4C}, R^{4D}, R^{4E}, R^{4F}, A¹, A², R^{1a1}, R^{1a2}, Y¹, Y², n₁, n², X, R^{A}, R^{A2}, R^{A3}, R^{A4}, R^{A5}, R^{A6}, and R^{A7} as if each combination had been individually and specifically described.

The embodiments also relate to pharmaceutically acceptable prodrugs of the compounds described herein, and treatment methods employing such pharmaceutically acceptable prodrugs. The term “prodrug” means a precursor of a designated compound that, following administration to a subject, yields the compound in vivo via a chemical or physiological process such as solvolysis or enzymatic cleavage, or under physiological conditions (e.g., a prodrug on being brought to physiological pH is converted to the compound of Formula (I)). A “pharmaceutically acceptable prodrug” is a prodrug that is non-toxic, biologically tolerable, and otherwise biologically suitable for administration to the subject. Illustrative procedures for the selection and preparation of suitable prodrug derivatives are described, for example, in “Design of Prodrugs”, ed. H. Bundgaard, Elsevier, 1985.

The embodiments also relate to pharmaceutically active metabolites of compounds described herein, and uses of such metabolites in the methods provided herein. A “pharmaceutically active metabolite” means a pharmacologically active product of metabolism in the body of a compound described herein or salt thereof. Prodrugs and active metabolites of a compound may be determined using routine techniques known or available in the art. See, e.g., Bertolini et al., J. Med. Chem. 1997, 40, 2011-2016; Shan et al., J. Pharm. Sci. 1997, 86 (7), 765-767; Bagshawe, Drug Dev. Res. 1995, 34, 220-230; Bodor, Adv. Drug Res. 1984, 13, 255-331; Bundgaard, Design of Prodrugs (Elsevier Press, 1985); and Larsen, Design and Application of Prodrugs, Drug Design and Development (Krogsgaard-Larsen et al., eds., Harwood Academic Publishers, 1991).

### Chemical Definitions

The following terms have the following meanings unless otherwise indicated. Any undefined terms have their art recognized meanings.

The term “alkyl” refers to a straight- or branched-chain univalent saturated hydrocarbon group, or combination thereof, having the number of carbon atoms designated (i.e., C₁-C₁₀ means one to ten carbon atoms). Examples of alkyl groups include, but are not limited to, methyl (Me), ethyl (Et), n-propyl, isopropyl, butyl, isobutyl, sec-butyl, tert-butyl (tBu), pentyl, isopentyl, tert-pentyl, hexyl, isohexyl, and groups that in light of the ordinary skill in the art and the teachings provided herein would be considered equivalent to any one of the foregoing examples.

The term “alkoxy” refers to an —O-alkyl. Examples of alkoxy include, but are not limited to, methoxy, ethoxy, n-propoxy, isopropoxy, n-butoxy, iso-butoxy, sec-butoxy, and tert-butoxy.

The term “alkenyl” refers to an unsaturated straight- or branched-chain hydrocarbon group, or combination thereof, having the indicated number of carbon atoms, and having one or more double bonds. Examples of alkenyl groups include, but are not limited to, ethenyl (or vinyl), allyl, and but-3-en-1-yl. Included within this term are cis and trans isomers and mixtures thereof.

The term “alkynyl” refers to an unsaturated straight- or branched-chain hydrocarbon group having the indicated number of carbon atoms (e.g., 2 to 8 or 2 to 6 carbon atoms) and at least one carbon-carbon triple bond. Examples of alkynyl groups include, but are not limited to, acetylenyl (—C≡CH) and propargyl (—CH2C≡CH).

The term “alkylene” refers to a divalent group that is a radical of an alkane. The alkylene can be a straight- or branched-chain divalent alkyl radical. “C₁₋₄ alkylene” refers to alkylene groups with 1 to 4 carbon atoms.

The term “aryl” refers to a monovalent aromatic carbocyclic group of from 6 to 18 annular carbon atoms having a single ring (a phenyl group) or a multiple condensed ring (such as napthyl, anthracenyl, or indanyl), in which condensed rings are optionally aromatic, provided that the point of attachment of the aryl group to the parent structure is through an atom of an aromatic ring. “Aryl” as defined herein encompasses groups such as phenyl and fluorenyl.

The term “cycloalkyl” refers to cyclic hydrocarbon groups of from 3 to 10 annular carbon atoms having single or multiple cyclic rings including fused, bridged, and spiro ring systems. Examples of suitable cycloalkyl groups include, for instance, adamantyl, cyclopropyl, cyclobutyl, cyclopentyl, cyclooctyl and the like. Such cycloalkyl groups include, by way of example, single ring structures such as cyclopropyl, cyclobutyl, cyclopentyl, cyclooctyl, and the like, or multiple ring structures such as adamantanyl, and the like. In some instances, the cycloalkyl is a monocyclic ring. In some instances, cycloalkyl is a 3- to 6-membered ring.

The term “cycloalkenyl” refers to a cyclic alkenyl group of from 4 to 10 annular carbon atoms having a single cyclic ring and at least one point of internal unsaturation which can be optionally substituted with from 1 to 3 alkyl groups. Examples of suitable cycloalkenyl groups include, for instance, cyclopent-3-enyl, cyclohex-2-enyl, cyclooct-3-enyl and the like.

The term “haloalkyl” refers to an alkyl group as described above, wherein one or more hydrogen atoms on the alkyl group have been replaced with a halo group. Examples of such groups include, without limitation, fluoroalkyl groups, such as fluoroethyl, trifluoromethyl, difluoromethyl, trifluoroethyl, and the like.

The term “heteroaryl” refers to a monocyclic, fused bicyclic, or fused polycyclic aromatic heterocycle (ring structure having ring atoms selected from carbon atoms and up to four heteroatoms selected from nitrogen, oxygen, and sulfur) having from 3 to 12 ring atoms per heterocycle. Illustrative examples of heteroaryl groups include the following entities, in the form of properly bonded moieties:

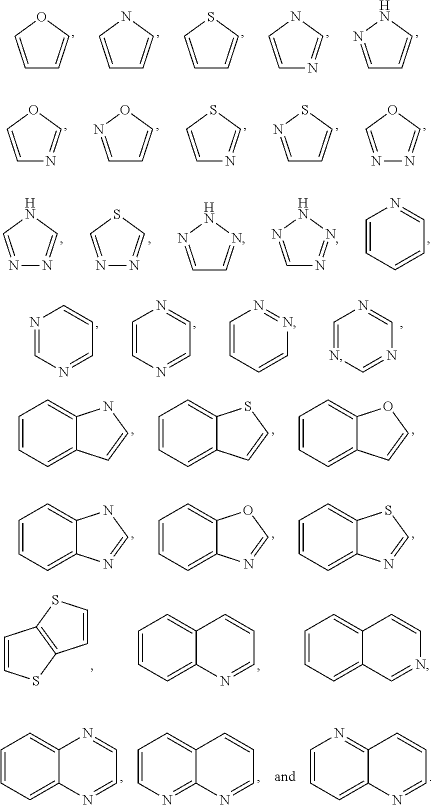

The terms “heterocyclyl” or “heterocycloalkyl” refer to a saturated or partially unsaturated group having a single ring or multiple condensed rings, including fused, bridged, or spiro ring systems, and having from 3 to 20 ring atoms, including 1 to 10 heteroatoms. These ring atoms are selected from the group consisting of carbon, nitrogen, sulfur, or oxygen. In certain embodiments, the nitrogen and/or sulfur atom(s) of the heterocyclic group are optionally oxidized to provide for N-oxide, —S(O)—, or —SO₂— moieties. Illustrative examples of heterocyclic groups include the following entities, in the form of properly bonded moieties:

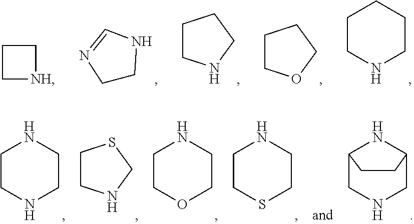

The term “halogen” represents chlorine, fluorine, bromine, or iodine. The term “halo” represents chloro, fluoro, bromo, or iodo.

The term “oxo” represents a carbonyl oxygen. For example, a cyclopentyl substituted with oxo is cyclopentanone.

Those skilled in the art will recognize that the species listed or illustrated above are not exhaustive, and that additional species within the scope of these defined terms may also be selected.

The term “substituted” means that the specified group or moiety bears one or more substituents including, but not limited to, substituents such as alkoxy, acyl, acyloxy, carbonylalkoxy, acylamino, amino, aminoacyl, aminocarbonylamino, aminocarbonyloxy, cycloalkyl, cycloalkenyl, aryl, heteroaryl, aryloxy, cyano, azido, halo, hydroxyl, nitro, carboxyl, thiol, thioalkyl, cycloalkyl, cycloalkenyl, alkyl, alkenyl, alkynyl, heterocyclyl, aralkyl, aminosulfonyl, sulfonylamino, sulfonyl, oxo, carbonylalkylenealkoxy and the like. The term “unsubstituted” means that the specified group bears no substituents. The term “optionally substituted” means that the specified group is unsubstituted or substituted by one or more substituents. Where the term “substituted” is used to describe a structural system, the substitution is meant to occur at any valency-allowed position on the system. When a group or moiety bears more than one substituent, it is understood that the substituents may be the same or different from one another. In some embodiments, a substituted group or moiety bears from one to five substituents. In some embodiments, a substituted group or moiety bears one substituent. In some embodiments, a substituted group or moiety bears two substituents. In some embodiments, a substituted group or moiety bears three substituents. In some embodiments, a substituted group or moiety bears four substituents. In some embodiments, a substituted group or moiety bears five substituents.

Any formula depicted herein is intended to represent a compound of that structural formula as well as certain variations or forms. For example, a formula given herein is intended to include a racemic form, or one or more enantiomeric, diastereomeric, or geometric isomers, or a mixture thereof. Additionally, any formula given herein is intended to refer also to a hydrate, solvate, or polymorph of such a compound, or a mixture thereof.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the present disclosure include isotopes of hydrogen, carbon, nitrogen, oxygen, phosphorous, fluorine, chlorine, and iodine, such as ²H, ³H, ¹¹C, ¹³C, ¹⁴C, ¹⁵N, ¹⁸O, ¹⁷O, ³¹P, ³²P, ³⁵S, ¹⁸F, ³⁶Cl, and ¹²⁵I, respectively. Such isotopically labelled compounds are useful in metabolic studies (preferably with ¹⁴C), reaction kinetic studies (with, for example ²H or ³H), detection or imaging techniques [such as positron emission tomography (PET) or single-photon emission computed tomography (SPECT)] including drug or substrate tissue distribution assays, or in radioactive treatment of patients. In particular, an ¹⁸F or ¹¹C labeled compound may be particularly preferred for PET or SPECT studies. PET and SPECT studies may be performed as described, for example, by Brooks, D. J., “Positron Emission Tomography and Single-Photon Emission Computed Tomography in Central Nervous System Drug Development,” NeuroRx 2005, 2(2), 226-236, and references cited therein. Further, substitution with heavier isotopes such as deuterium (i.e., ²H) may afford certain therapeutic advantages resulting from greater metabolic stability, for example increased in vivo half-life or reduced dosage requirements. Isotopically labeled compounds of the present disclosure and prodrugs thereof can generally be prepared by carrying out the procedures disclosed in the schemes or in the examples and preparations described below by substituting a readily available isotopically labeled reagent for a non-isotopically labeled reagent.

The nomenclature “Cᵢ₋ⱼ” with j>i, when applied herein to a class of substituents, is meant to refer to embodiments of the present disclosure for which each and every one of the number of carbon members, from i to j including i and j, is independently realized. By way of example, the term C₁₋₃ refers independently to embodiments that have one carbon member (C₁), embodiments that have two carbon members (C₂), and embodiments that have three carbon members (C₃).

The present disclosure also includes pharmaceutically acceptable salts of the compounds represented by Formula (I), or the compounds of Table 1, and pharmaceutical compositions comprising such salts, and methods of using such salts.

A “pharmaceutically acceptable salt” is intended to mean a salt of a free acid or base of a compound represented herein that is non-toxic, biologically tolerable, or otherwise biologically suitable for administration to the subject. See, generally, S. M. Berge, et al., “Pharmaceutical Salts,” J. Pharm. Sci., 1977, 66, 1-19. Particular pharmaceutically acceptable salts are those that are pharmacologically effective and suitable for contact with the tissues of subjects without undue toxicity, irritation, or allergic response. A compound described herein may possess a sufficiently acidic group, a sufficiently basic group, both types of functional groups, or more than one of each type, and accordingly react with a number of inorganic or organic bases, and inorganic and organic acids, to form a pharmaceutically acceptable salt.

Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, 7-hydroxybutyrates, glycolates, tartrates, and mandelates. Lists of other suitable pharmaceutically acceptable salts are found in Remington's Pharmaceutical Sciences, 17th Edition, Mack Publishing Company, Easton, Pa., 1985.

For a compound of Formula (I), or a compound of Table 1 that contains a basic nitrogen, a pharmaceutically acceptable salt may be prepared by any suitable method available in the art, for example, treatment of the free base with an inorganic acid, such as hydrochloric acid, hydrobromic acid, sulfuric acid, sulfamic acid, nitric acid, boric acid, phosphoric acid, and the like, or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid or glutamic acid, an aromatic acid, such as benzoic acid, 2-acetoxybenzoic acid, naphthoic acid, or cinnamic acid, a sulfonic acid, such as laurylsulfonic acid, p-toluenesulfonic acid, methanesulfonic acid, or ethanesulfonic acid, or any compatible mixture of acids such as those given as examples herein, and any other acid and mixture thereof that are regarded as equivalents or acceptable substitutes in light of the ordinary level of skill in this technology.

The present disclsoure also relates to pharmaceutically acceptable prodrugs of the compounds of Formula (I), or the compounds of Table 1, and treatment methods employing such pharmaceutically acceptable prodrugs. The term “prodrug” means a precursor of a designated compound that, following administration to a subject, yields the compound in vivo via a chemical or physiological process such as solvolysis or enzymatic cleavage, or under physiological conditions (e.g., a prodrug on being brought to physiological pH is converted to the formula compound). A “pharmaceutically acceptable prodrug” is a prodrug that is non-toxic, biologically tolerable, and otherwise biologically suitable for administration to the subject. Illustrative procedures for the selection and preparation of suitable prodrug derivatives are described, for example, in “Design of Prodrugs”, ed. H. Bundgaard, Elsevier, 1985.

The present disclosure also relates to pharmaceutically active metabolites of compounds of Formula (I), or the compounds of Table 1, and uses of such metabolites in the methods provided herein. A “pharmaceutically active metabolite” means a pharmacologically active product of metabolism in the body of a compound of Formula (I), or the compounds of Table 1, or a salt of any of the foregoing. Prodrugs and active metabolites of a compound may be determined using routine techniques known or available in the art. See, e.g., Bertolini et al., J. Med. Chem. 1997, 40, 2011-2016; Shan et al., J. Pharm. Sci. 1997, 86 (7), 765-767; Bagshawe, Drug Dev. Res. 1995, 34, 220-230; Bodor, Adv. Drug Res. 1984, 13, 255-331; Bundgaard, Design of Prodrugs (Elsevier Press, 1985); and Larsen, Design and Application of Prodrugs, Drug Design and Development (Krogsgaard-Larsen et al., eds., Harwood Academic Publishers, 1991).

Pharmaceutical Compositions

For treatment purposes, pharmaceutical compositions comprising the compounds described herein may further comprise one or more pharmaceutically-acceptable excipients. A pharmaceutically-acceptable excipient is a substance that is non-toxic and otherwise biologically suitable for administration to a subject. Such excipients facilitate administration of the compounds described herein and are compatible with the active ingredient. Examples of pharmaceutically-acceptable excipients include stabilizers, lubricants, surfactants, diluents, anti-oxidants, binders, coloring agents, bulking agents, emulsifiers, or taste-modifying agents. In particular embodiments, pharmaceutical compositions according to the present disclosure are sterile compositions. Pharmaceutical compositions may be prepared using compounding techniques known or that become available to those skilled in the art.

Sterile compositions are also contemplated by the present disclosure, including compositions that are in accord with national and local regulations governing such compositions.

The pharmaceutical compositions and compounds described herein may be formulated as solutions, emulsions, suspensions, or dispersions in suitable pharmaceutical solvents or carriers, or as pills, tablets, lozenges, suppositories, sachets, dragees, granules, powders, powders for reconstitution, or capsules along with solid carriers according to conventional methods known in the art for preparation of various dosage forms. Pharmaceutical compositions of the present disclosure may be administered by a suitable route of delivery, such as oral, parenteral, rectal, nasal, topical, or ocular routes, or by inhalation. In some embodiments, the compositions are formulated for intravenous or oral administration.

For oral administration, the compounds of the present disclosure may be provided in a solid form, such as a tablet or capsule, or as a solution, emulsion, or suspension. To prepare the oral compositions, the compounds of the present disclosure may be formulated to yield a dosage of, e.g., from about 0.01 to about 50 mg/kg daily, or from about 0.05 to about 20 mg/kg daily, or from about 0.1 to about 10 mg/kg daily. Additional dosages include from about 0.1 mg to 1 g daily, from about 1 mg to about 10 mg daily, from about 10 mg to about 50 mg daily, from about 50 mg to about 250 mg daily, or from about 250 mg to 1 g daily. Oral tablets may include the active ingredient(s) mixed with compatible pharmaceutically acceptable excipients such as diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are exemplary disintegrating agents. Binding agents may include starch and gelatin. The lubricating agent, if present, may be magnesium stearate, stearic acid, or talc. If desired, the tablets may be coated with a material such as glyceryl monostearate or glyceryl distearate to delay absorption in the gastrointestinal tract, or may be coated with an enteric coating.

Capsules for oral administration include hard and soft gelatin capsules. To prepare hard gelatin capsules, active ingredient(s) may be mixed with a solid, semi-solid, or liquid diluent. Soft gelatin capsules may be prepared by mixing the active ingredient with water, an oil such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions, or syrups, or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

The inventive compositions may be formulated for rectal administration as a suppository. For parenteral use, including intravenous, intramuscular, intraperitoneal, intranasal, or subcutaneous routes, the agents of the present disclosure may be provided in sterile aqueous solutions or suspensions, buffered to an appropriate pH and isotonicity or in parenterally acceptable oil. Suitable aqueous vehicles include Ringer's solution and isotonic sodium chloride. Such forms may be presented in unit-dose form such as ampoules or disposable injection devices, in multi-dose forms such as vials from which the appropriate dose may be withdrawn, or in a solid form or pre-concentrate that can be used to prepare an injectable formulation. Illustrative infusion doses range from about 1 to 1000 μg/kg/minute of agent admixed with a pharmaceutical carrier over a period ranging from several minutes to several days.

For nasal, inhaled, or oral administration, the inventive pharmaceutical compositions may be administered using, for example, a spray formulation also containing a suitable carrier.

For topical applications, the compounds of the present disclosure may be formulated as creams or ointments or a similar vehicle suitable for topical administration. For topical administration, the inventive compounds may be mixed with a pharmaceutical carrier at a concentration of about 0.1% to about 10% of drug to vehicle. Another mode of administering the agents of the present disclosure may utilize a patch formulation to effect transdermal delivery.

As used herein, “treatment” or “treating” is an approach for obtaining a beneficial or desired result, including clinical results. For purposes of this disclosure, beneficial or desired results include, but are not limited to: reducing the severity of or suppressing the worsening of a disease, symptom, or condition, alleviating a symptom and/or diminishing the extent of a symptom and/or preventing a worsening of a symptom associated with a condition, arresting the development of a disease, symptom, or condition, relieving the disease, symptom, or condition, causing regression of the disease, disorder, or symptom (in terms of severity or frequency of negative symptoms), or stopping the symptoms of the disease or condition. Beneficial or desired results can also be slowing, halting, or reversing the progressive course of a disease or condition. For example, beneficial effects may include slowing the progression of Parkinson's disease from an earlier stage (e.g., prodromal stage or stage 1, 2 or 3) to a later stage (e.g., stage 4 or 5), or halting Parkinson's disease at a prodromal or early stage.

As used herein, “delaying” development of a disease or condition means to defer, hinder, slow, retard, stabilize and/or postpone development of the disease or condition. This delay can be of varying lengths of time, depending on the history of the disease and/or individual being treated. As is evident to one skilled in the art, a sufficient or significant delay can, in effect, encompass prevention, in that the individual does not develop the disease or condition. For example, a method that “delays” development of Parkinson's disease (e.g., in a prodromal individual) is a method that reduces probability of disease development in a given time frame and/or reduces extent of the disease in a given time frame, when compared to not using the method.

The term “subject” refers to a mammalian patient in need of such treatment, such as a human. A “subject” may be a human, or may be a cat, dog, cow, rat, mouse, horse, rabbit, or other domesticated mammal.

Exemplary diseases that are characterized by protein aggregation include Alzheimer's disease, Parkinson's disease, fronto-temporal dementia, dementia with Lewy bodies (Lewy body disease), Parkinson's disease with dementia, multiple system atrophy, amyotrophic lateral sclerosis, Huntington's disease, Progressive Supranuclear Palsy (PSP), and Niemann-Pick disease type C, Guillain-Barré syndrome (GBS), Barrett's esophagus, as well inflammatory diseases, such as asthma, chronic obstructive pulmonary disease (COPD), chronic peptic ulcers, irritable bowel disease, tuberculosis, rheumatoid arthritis, osteoarthritis, chronic sinusitis, hepatitis (such as hepatitis B or C), gout, lupus, pleurisy, eczema, gastritis, psoriasis, psoriatic arthritis, vasculitis, laryngitis, allergic reactions, multiple sclerosis, Crohn's disease, traumatic brain injury, CIDP (chronic inflammatory demyelinating polyneuropathy), stroke, ischemic heart disease, atopic dermatitis, acne vulgaris, rosacea, non-alcoholic fatty liver disease, non-alcoholic steatohepatisis, corneal wounds, corneal disorders, corneal HSV, Stargardt disease (Juvenile macular degeneration), age-related macular degeneration, sepsis, diabetic wounds, herpes simplex virus, and anti-fungal, anti-bacterial, anitviral and antitumor diseases or conditions.

In one aspect, the compounds and pharmaceutical compositions of the present disclosure specifically target TLR2 protein dimers. Thus, these compounds and pharmaceutical compositions can be used to prevent, reverse, slow, or inhibit dimerization of TLR2 proteins with other natural protein ligands, and are used in methods of the present disclosure to treat neurological and inflammatory diseases related to or caused by such dimerization. In some embodiments, methods of treatment target Parkinson's disease, Alzheimer's disease, Lewy body disease, multiple system atrophy, atopic dermatitis, traumatic brain injury, or multiple sclerosis. The compounds, compositions, and method of the present disclosure are also used to mitigate deleterious effects that are secondary to protein dimerization and/or misfolding, such as neuronal cell death. In another aspect, the compounds and pharmaceutical compositions of the present disclosure are inhibitors of TLR9. In some embodiments, the compounds and pharmaceutical compositions of the present disclosure are used in methods of the present disclosure to treat central nervous system (CNS) and peripheral disorders. In some embodiments, methods of treatment target Parkinson's disease, Amyotrophic lateral sclerosis, Guillain-Barre syndrome, spinal cord injury, multiple sclerosis, multiple forms of tissue injury, chronic pain, or psoriasis.

In some aspects, the compounds, compositions, and methods of the present disclosure are used to inhibit TLR2 dimerization. In alternative aspects, the compounds, compositions, and methods of the present disclosure are used to inhibit TLR2 dimerization with TLR1, or with TLR6, or both.

In the inhibitory methods of the present disclosure, an “effective amount” means an amount sufficient to reduce, slow the progression of, or reverse TLR2 dimerization. Measuring the amount of dimerization may be performed by routine analytical methods such as those described below. Such modulation is useful in a variety of settings, including in vitro assays. In some embodiments of such methods, the cell is a nerve cell or an HEK or THP cell.

In treatment methods according to the present disclosure, an “effective amount” means an amount or dose sufficient to generally bring about the desired therapeutic benefit in subjects needing such treatment. Effective amounts or doses of the compounds of the present disclosure may be ascertained by routine methods, such as modeling, dose escalation, or clinical trials, taking into account routine factors, e.g., the mode or route of administration or drug delivery, the pharmacokinetics of the agent, the severity and course of the infection, the subject's health status, condition, and weight, and the judgment of the treating physician. An exemplary dose is in the range of about 1 μg to 2 mg of active agent per kilogram of subject's body weight per day, such as about 0.05 to 100 mg/kg/day, or about 1 to 35 mg/kg/day, or about 0.1 to 10 mg/kg/day. In alternative embodiments an exemplary dose is in the range of about 1 mg to about 1 g per day, or about 1-500, 1-250, 1-100, 1-50, 50-500, or 250-500 mg per day. The total dosage may be given in single or divided dosage units (e.g., BID, TID, QID).

Once improvement of the patient's disease has occurred, the dose may be adjusted for preventative or maintenance treatment. For example, the dosage or the frequency of administration, or both, may be reduced as a function of the symptoms, to a level at which the desired therapeutic or prophylactic effect is maintained. Of course, if symptoms have been alleviated to an appropriate level, treatment may cease. Patients may, however, require intermittent treatment on a long-term basis upon any recurrence of symptoms. Patients may also require chronic treatment on a long-term basis.

Drug Combinations

The inventive compounds described herein may be used in pharmaceutical compositions or methods in combination with one or more additional active ingredients in the treatment of neurodegenerative disorders. Further additional active ingredients for cancer applications include other cancer therapeutics or agents that mitigate adverse effects of cancer chemotherapeutic agents. Such combinations may serve to increase efficacy, ameliorate other disease symptoms, decrease one or more side effects, or decrease the required dose of an inventive compound. The additional active ingredients may be administered in a separate pharmaceutical composition from a compound of the present disclosure or may be included with a compound of the present disclosure in a single pharmaceutical composition. The additional active ingredients may be administered simultaneously with, prior to, or after administration of a compound of the present disclosure.

Combination agents include additional active ingredients are those that are known or discovered to be effective in treating the diseases, disorders, conditions, and symptoms discussed herein, including those active against another target associated with the disease, disorder, or symptom such as but not limited to, a) compounds that address protein misfolding (such as drugs which reduce the production of these proteins, which increase their clearance or which alter their aggregation and/or propagation); b) compounds that treat symptoms of such disorders (e.g., dopamine replacement therapies); and c) drugs that act as neuroprotectants by complementary mechanisms (e.g., those targeting autophagy, those that are anti-oxidants, and those acting by other mechanisms such as adenosine A2A antagonists).

For example, compositions and formulations of the present disclosure, as well as methods of treatment, can further comprise other drugs or pharmaceuticals, e.g., other active agents useful for treating or palliative for a neurological or inflammatory diseases related to or caused by TLR2 dimerization, e.g., Parkinson's disease, Alzheimer's Disease (AD), Lewy body disease (LBD) and multiple system atrophy (MSA), or related symptoms or conditions. For example, the pharmaceutical compositions of the present disclosure may additional comprise one or more of such active agents, and methods of treatment may additionally comprise administering an effective amount of one or more of such active agents. In certain embodiments, additional active agents may be antibiotics (e.g., antibacterial or bacteriostatic peptides or proteins), e.g., those effective against gram positive or negative bacteria, fluids, cytokines, immunoregulatory agents, anti-inflammatory agents, complement activating agents, such as peptides or proteins comprising collagen-like domains or fibrinogen-like domains (e.g., a ficolin), carbohydrate-binding domains, and the like and combinations thereof. Additional active agents include those useful in such compositions and methods include dopamine therapy drugs, catechol-O-methyl transferase (COMT) inhibitors, monamine oxidase inhibitors, cognition enhancers (such as acetylcholinesterase inhibitors or memantine), adenosine 2A receptor antagonists, beta-secretase inhibitors, or gamma-secretase inhibitors. In particular embodiments, at least one compound of the present disclosure may be combined in a pharmaceutical composition or a method of treatment with one or more drugs selected from the group consisting of: tacrine (Cognex), donepezil (Aricept), rivastigmine (Exelon) galantamine (Reminyl), physostigmine, neostigmine, Icopezil (CP-118954, 5,7-dihydro-3-[2-[1-(phenylmethyl)-4-piperidinyl]ethyl]-6H-pyrrolo-[4,5-f-]-1,2-benzisoxazol-6-one maleate), ER-127528 (4-[(5,6-dimethoxy-2-fluoro-1-indanon)-2-yl]methyl-1-(3-fluorobenzyl)piperidine hydrochloride), zanapezil (TAK-147; 3-[1-(phenylmethyl)piperidin-4-yl]-1-(2,3,4,5-tetrahydro-1H-1-benzazepin-8-yl)-1-propane fumarate), Metrifonate (T-588; (−)-R-.alpha.-[[2-(dimethylamino)ethoxy]methyl]benzo[b]thiophene-5-methanol hydrochloride), FK-960 (N-(4-acetyl-1-piperazinyl)-p-fluorobenzamide-hydrate), TCH-346 (N-methyl-N-2-pyropinyldibenz[b,f]oxepine-10-methanamine), SDZ-220-581 ((S)-alpha-amino-5-(phosphonomethyl)-[1,1′-biphenyl]-3-propionic acid), memantine (Namenda/Exiba) and 1,3,3,5,5-pentamethylcyclohexan-1-amine (Neramexane), tarenflurbil (Flurizan), tramiprosate (Alzhemed), clioquinol, PBT-2 (an 8-hydroxyquinilone derivative), 1-(2-(2-Naphthyl)ethyl)-4-(3-trifluoromethylphenyl)-1,2,3,6-tetrahydropyridine, Huperzine A, posatirelin, leuprolide or derivatives thereof, ispronicline, (3-aminopropyl)(n-butyl)phosphinic acid (SGS-742), N-methyl-5-(3-(5-isopropoxypyridinyl))-4-penten-2-amine (ispronicline), 1-decanaminium, N-(2-hydroxy-3-sulfopropyl)-N-methyl-N-octyl-, inner salt (zt-1), salicylates, aspirin, amoxiprin, benorilate, choline magnesium salicylate, diflunisal, faislamine, methyl salicylate, magnesium salicylate, salicyl salicylate, diclofenac, aceclofenac, acemetacin, bromfenac, etodolac, indometacin, nabumetone, sulindac, tolmetin, ibuprofen, carprofen, fenbufen, fenoprofen, flurbiprofen, ketoprofen, ketorolac, loxoprofen, naproxen, tiaprofenic acid, suprofen, mefenamic acid, meclofenamic acid, phenylbutazone, azapropazone, metamizole, oxyphenbutazone, sulfinprazone, piroxicam, lornoxicam, meloxicam, tenoxicam, celecoxib, etoricoxib, lumiracoxib, parecoxib, rofecoxib, valdecoxib, nimesulide, arylalkanoic acids, 2-arylpropionic acids (profens), N-arylanthranilic acids (fenamic acids), pyrazolidine derivatives, oxicams, COX-2 inhibitors, sulphonanilides, essential fatty acids, and Minozac (2-(4-(4-methyl-6-phenylpyridazin-3-yl)piperazin-1-yl)pyrimidine dihydrochloride hydrate), or a combination thereof.

### Methods of Use

The compounds and pharmaceutical compositions herein may be used to treat or prevent a disease or condition in an individual. In some embodiments, provided are methods of treating a disease or condition associated with TLR2 or TLR2 heterodimerization, comprising administering to the individual in need thereof a compound of Formula (I), or a compound of Table 1, or a tautomer thereof, or a pharmaceutically acceptable salt of any of the foregoing. In some embodiments, provided are methods of treating a disease or condition associated with TLR2 or TLR2 heterodimerization comprising administering to the subject a therapeutically effective amount of at least one chemical entity as described herein.

In some embodiments, provided are compositions containing one or more compounds of Formula (I), or a compound of Table 1, or a tautomer thereof, or a pharmaceutically acceptable salt of any of the foregoing, for use in the treatment of a disease or condition associated with TLR2 or TLR2 heterodimerization. In some embodiments, provided are compositions containing at least one chemical entity as described herein for use in the treatment of a disease or condition associated with TLR2 or TLR2 heterodimerization.

Also provided herein is the use of a compound of Formula (I), or a compound of Table 1, or a tautomer thereof, or a pharmaceutically acceptable salt of any of the foregoing, in the manufacture of a medicament for treatment of a disease or condition associated with TLR2 or TLR2 heterodimerization. In some embodiments, provided is the use of at least one chemical entity as described herein in the manufacture of a medicament for treatment of a disease or condition associated with TLR2 or TLR2 heterodimerization.

In some embodiments, the disease or condition is selected from Alzheimer's disease, Parkinson's disease, fronto-temporal dementia, dementia with Lewy bodies (Lewy body disease), Parkinson's disease with dementia, multiple system atrophy, amyotrophic lateral sclerosis, Huntington's disease, Progressive Supranuclear Palsy (PSP), Niemann-Pick disease type C, Guillain-Barré syndrome (GBS), Barrett's esophagus, inflammatory diseases, asthma, chronic obstructive pulmonary disease (COPD), chronic peptic ulcers, irritable bowel disease, tuberculosis, rheumatoid arthritis, osteoarthritis, chronic sinusitis, hepatitis, hepatitis B, hepatitis C, gout, lupus, pleurisy, eczema, gastritis, psoriasis, psoriatic arthritis, vasculitis, laryngitis, allergic reactions, multiple sclerosis, Crohn's disease, traumatic brain injury, CIDP (chronic inflammatory demyelinating polyneuropathy), stroke, ischemic heart disease, atopic dermatitis, acne vulgaris, rosacea, non-alcoholic fatty liver disease, non-alcoholic steatohepatisis, corneal wounds, corneal disorders, corneal HSV, Stargardt disease (Juvenile macular degeneration), age-related macular degeneration, sepsis, diabetic wounds, herpes simplex virus, and anti-fungal, anti-bacterial, anitviral and antitumor diseases or conditions. In some embodiments, the disease or condition is selected from Alzheimer's disease, Parkinson's disease, fronto-temporal dementia, dementia with Lewy bodies (Lewy body disease), Parkinson's disease with dementia, multiple system atrophy, amyotrophic lateral sclerosis, Huntington's disease, inflammatory diseases, asthma, chronic obstructive pulmonary disease (COPD), chronic peptic ulcers, tuberculosis, rheumatoid arthritis, chronic sinusitis, hepatitis, hepatitis B, hepatitis C, gout, lupus, pleurisy, eczema, gastritis, psoriasis, psoriatic arthritis, vasculitis, laryngitis, allergic reactions, multiple sclerosis, Crohn's disease, and traumatic brain injury.

Also provided are methods for interfering with the heterodimerization of TLR2 in a cell, or modulating, preventing, slowing, reversing, or inhibiting TLR2 heterodimerization in a cell which involves contacting the cell with an effective amount of at least one compound of Formula (I), or a compound of Table 1, or a tautomer thereof, or a pharmaceutically acceptable salt of any of the foregoing. In some embodiments, provided are methods for interfering with the heterodimerization of TLR2 in a cell, or modulating, preventing, slowing, reversing, or inhibiting TLR2 heterodimerization in a cell which involves contacting the cell with an effective amount of at least one chemical entity as described herein. In some embodiments, provided are methods of inhibiting TLR2 activation in a cell, comprising contacting the cell with an effective amount of at least one compound of Formula (I), or a compound of Table 1, or a tautomer thereof, or a pharmaceutically acceptable salt of any of the foregoing, or a pharmaceutical composition comprising any of the foregoing, wherein the contacting is in vitro, ex vivo, or in vivo.

Also provided herein are compositions containing one or more compounds of Formula (I), or a compound of Table 1, or a tautomer thereof, or a pharmaceutically acceptable salt of any of the foregoing, for use in interfering with the heterodimerization of TLR2 in a cell, or modulating, preventing, slowing, reversing, or inhibiting TLR2 heterodimerization in a cell. In some embodiments, provided are compositions containing at least one chemical entity as described herein for use in interfering with the heterodimerization of TLR2 in a cell, or modulating, preventing, slowing, reversing, or inhibiting TLR2 heterodimerization in a cell.

Additionally provided herein is the use of at least one chemical entity as described herein, such as a compound of Formula (I), or a compound of Table 1, or a tautomer thereof, or a pharmaceutically acceptable salt of any of the foregoing, in the manufacture of a medicament for interfering with the heterodimerization of TLR2, or modulating, preventing, slowing, reversing, or inhibiting TLR2 heterodimerization.

In some embodiments, provided are methods of treating a disease or condition associated with inhibition of TLR9, comprising administering to the individual in need thereof a compound of Formula (I), or a compound of Table 1, or a tautomer thereof, or a pharmaceutically acceptable salt of any of the foregoing.

In some embodiments, provided are compositions containing one or more compounds of Formula (I), or a compound of Table 1, or a tautomer thereof, or a pharmaceutically acceptable salt of any of the foregoing, for use in the treatment of a disease or condition associated with inhibition of TLR9. In some embodiments, provided are compositions containing at least one chemical entity as described herein for use in the treatment of a disease or condition associated with inhibition of TLR9.

Also provided herein is the use of a compound of Formula (I), or a compound of Table 1, or a tautomer thereof, or a pharmaceutically acceptable salt of any of the foregoing, in the manufacture of a medicament for treatment of a disease or condition associated with inhibition of TLR9. In some embodiments, provided is the use of at least one chemical entity as described herein in the manufacture of a medicament for treatment of a disease or condition associated with inhibition of TLR9.

In some embodiments, the disease or condition is central nervous sytem (CNS) or peripheral disorder. In some embodiments, the disease or condition is Parkinson's disease, Amyotrophic lateral sclerosis, Guillain-Barre syndrome, spinal cord injury, multiple sclerosis, multiple forms of tissue injury, chronic pain, or psoriasis.

Also provided are methods of inhibiting TLR9 in a cell, which involves contacting the cell with an effective amount of at least one compound of Formula (I), or a compound of Table 1, or a tautomer thereof, or a pharmaceutically acceptable salt of any of the foregoing. In some embodiments, provided are methods for inhibiting TLR9 in a cell, which involves contacting the cell with an effective amount of at least one chemical entity as described herein. In some embodiments, provided are methods of inhibiting TLR9 activation in a cell, comprising contacting the cell with an effective amount of at least one compound of Formula (I), or a compound of Table 1, or a tautomer thereof, or a pharmaceutically acceptable salt of any of the foregoing, or a pharmaceutical composition comprising any of the foregoing, wherein the contacting is in vitro, ex vivo, or in vivo.

Also provided herein are compositions containing one or more compounds of Formula (I), or a compound of Table 1, or a tautomer thereof, or a pharmaceutically acceptable salt of any of the foregoing, for use in inhibiting TLR9 in a cell. In some embodiments, provided are compositions containing at least one chemical entity as described herein for use in inhibiting TLR9 in a cell.

Additionally provided herein is the use of at least one chemical entity as described herein, such as a compound of Formula (I), or a compound of Table 1, or a tautomer thereof, or a pharmaceutically acceptable salt of any of the foregoing, in the manufacture of a medicament for inhibiting TLR9.

In some embodiments, compounds described herein, such as a compound of Formula (I), or a compound of Table 1, or a tautomer thereof, or a pharmaceutically acceptable salt of any of the foregoing, inhibit both TLR2 and TLR9. In some embodiments, provided are methods of treating a disease or condition associated with TLR2 or TLR2 heterodimerization and/or inhibition of TLR9, comprising administering to the individual in need thereof a compound of Formula (I), or a compound of Table 1, or a tautomer thereof, or a pharmaceutically acceptable salt of any of the foregoing. In some embodiments, provided are methods of treating a disease or condition associated with TLR2 or TLR2 heterodimerization and/or inhibition of TLR9 comprising administering to the subject a therapeutically effective amount of at least one chemical entity as described herein.

In some embodiments, provided are compositions containing one or more compounds of Formula (I), or a compound of Table 1, or a tautomer thereof, or a pharmaceutically acceptable salt of any of the foregoing, for use in the treatment of a disease or condition associated with TLR2, TLR2 heterodimerization and/or inhibition of TLR9. In some embodiments, provided are compositions containing at least one chemical entity as described herein for use in the treatment of a disease or condition associated with TLR2, TLR2 heterodimerization and/or inhibition of TLR9.

Also provided herein is the use of a compound of Formula (I), or a compound of Table 1, or a tautomer thereof, or a pharmaceutically acceptable salt of any of the foregoing, in the manufacture of a medicament for treatment of a disease or condition associated with TLR2, TLR2 heterodimerization and/or inhibition of TLR9. In some embodiments, provided is the use of at least one chemical entity as described herein in the manufacture of a medicament for treatment of a disease or condition associated with TLR2, TLR2 heterodimerization and/or inhibition of TLR9.

### Kits

Also provided are articles of manufacture and kits containing any of the compounds or pharmaceutical compositions provided herein. The article of manufacture may comprise a container with a label. Suitable containers include, for example, bottles, vials, and test tubes. The containers may be formed from a variety of materials such as glass or plastic. The container may hold a pharmaceutical composition provided herein. The label on the container may indicate that the pharmaceutical composition is used for preventing, treating or suppressing a condition described herein, and may also indicate directions for either in vivo or in vitro use.

In one aspect, provided herein are kits containing a compound or composition described herein and instructions for use. The kits may contain instructions for use in the treatment of a disease or condition associated with TLR2 or TLR2 heterodimerization in an individual in need thereof and/or instructions for use in the treatment of a disease or condition associated with inhibition of TLR9 in an individual in need thereof. A kit may additionally contain any materials or equipment that may be used in the administration of the compound or composition, such as vials, syringes, or IV bags. A kit may also contain sterile packaging.

### General Synthetic Methods

The compounds of the present disclosure may be prepared by a number of processes as generally described below and more specifically in the Examples hereinafter (such as the schemes provided in the Examples below). In the following process descriptions, the symbols when used in the formulae depicted are to be understood to represent those groups described above in relation to the formulae herein.

Where it is desired to obtain a particular enantiomer of a compound, this may be accomplished from a corresponding mixture of enantiomers using any suitable conventional procedure for separating or resolving enantiomers. Thus, for example, diastereomeric derivatives may be produced by reaction of a mixture of enantiomers, e.g., a racemate, and an appropriate chiral compound. The diastereomers may then be separated by any convenient means, for example by crystallization and the desired enantiomer recovered. In another resolution process, a racemate may be separated using chiral High Performance Liquid Chromatography. Alternatively, if desired a particular enantiomer may be obtained by using an appropriate chiral intermediate in one of the processes described.

Chromatography, recrystallization and other conventional separation procedures may also be used with intermediates or final products where it is desired to obtain a particular isomer of a compound or to otherwise purify a product of a reaction.

Solvates of a compound provided herein or a pharmaceutically acceptable salt thereof are also contemplated. Solvates contain either stoichiometric or non-stoichiometric amounts of a solvent, and are often formed during the process of crystallization. Hydrates are formed when the solvent is water, or alcoholates are formed when the solvent is alcohol.

In some embodiments, compounds of Formula (I) may be synthesized according to Scheme 1.

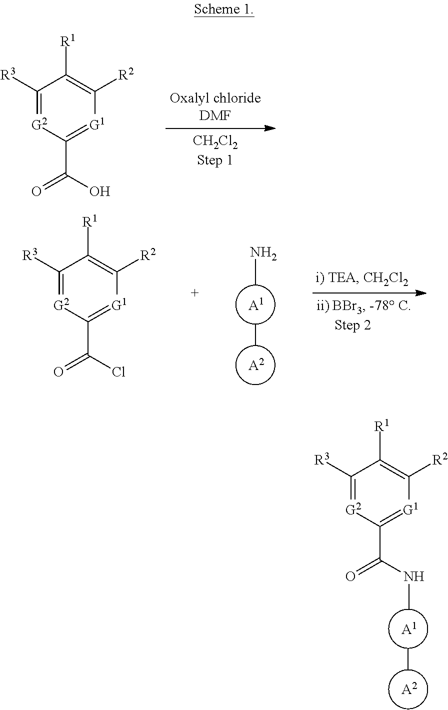

wherein R¹, R², R³, G¹, G², A¹, and A² are as defined for Formula (I), or any variation thereof detailed herein.

In some embodiments of the foregoing Scheme 1, compounds of Formula (I) may be synthesized according to Scheme 1-1.

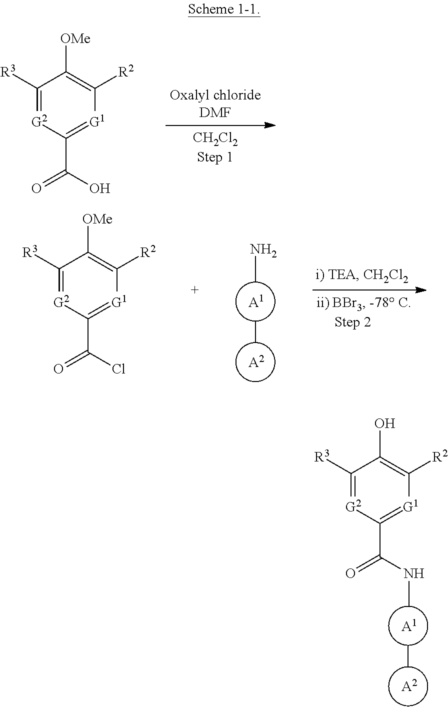

wherein R², R³, G¹, G², A¹, and A² are as defined for Formula (I), or any variation thereof detailed herein.

In some variations of the foregoing Scheme 1, compounds of Formula (I) may be synthesized according to Scheme 1-2.

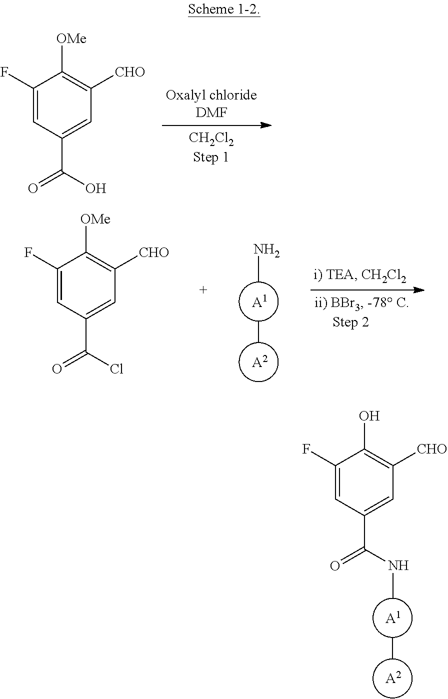

wherein A¹ is optionally substituted 5-membered heteroaryl and A² is optionally substituted 6-membered heterocyclyl or optionally substituted phenyl.

In some embodiments, compounds of Formula (I) may be synthesized according to Scheme 2.

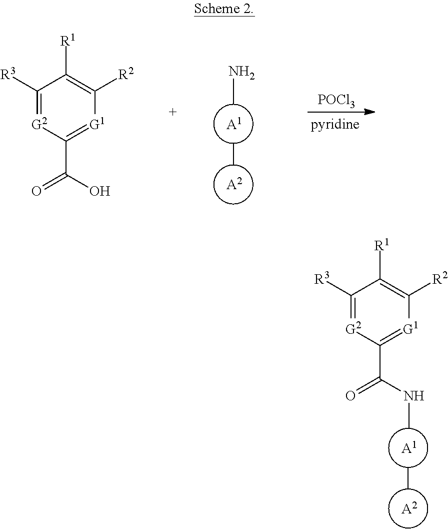

wherein R¹, R², R³, G¹, G², A¹, and A² are as defined for Formula (I), or any variation thereof detailed herein.

In some variations of the foregoing Scheme 2, compounds of Formula (I) may be synthesized according to Scheme 2-1.

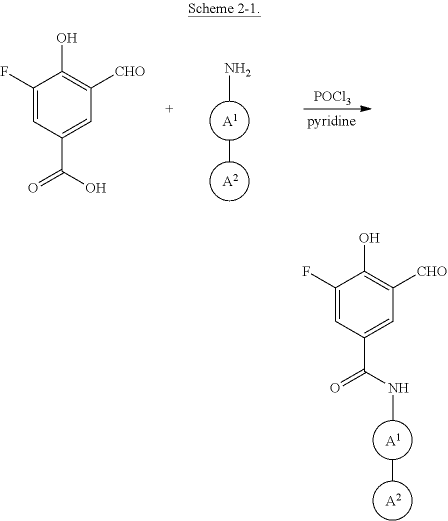

wherein A¹ is optionally substituted 5-membered heteroaryl and A² is optionally substituted 6-membered heterocyclyl or optionally substituted phenyl.

In some embodiments, compounds of Formula (I) may be synthesized according to Scheme 3.

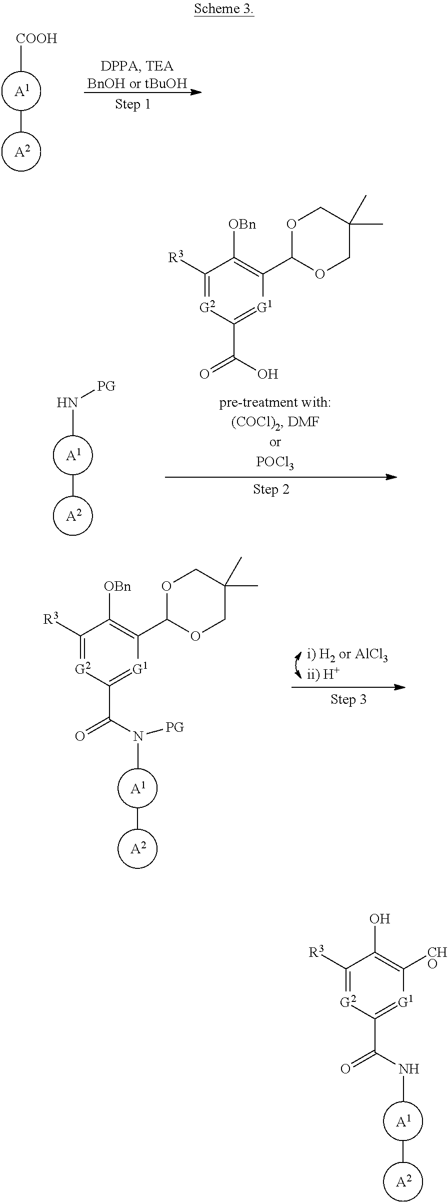

wherein R³, G¹, G², A¹, and A² are as defined for Formula (I), or any variation thereof detailed herein; and PG is is Boc (C(O)OtBu) or CBZ (C(O)OBn).

In some variations of the foregoing Scheme 3, compounds of Formula (I) may be synthesized according to Scheme 3-1.

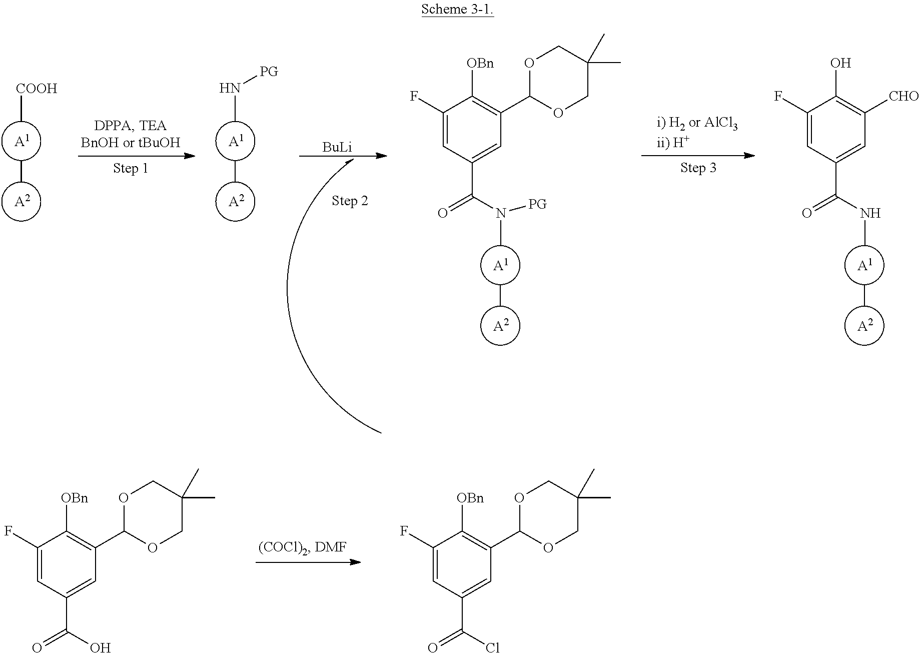

wherein A¹ is optionally substituted 5-membered heteroaryl and A² is optionally substituted heterocyclyl or optionally substituted aryl.

In some embodiments, compounds of Formula (I) may be synthesized according to Scheme 4.

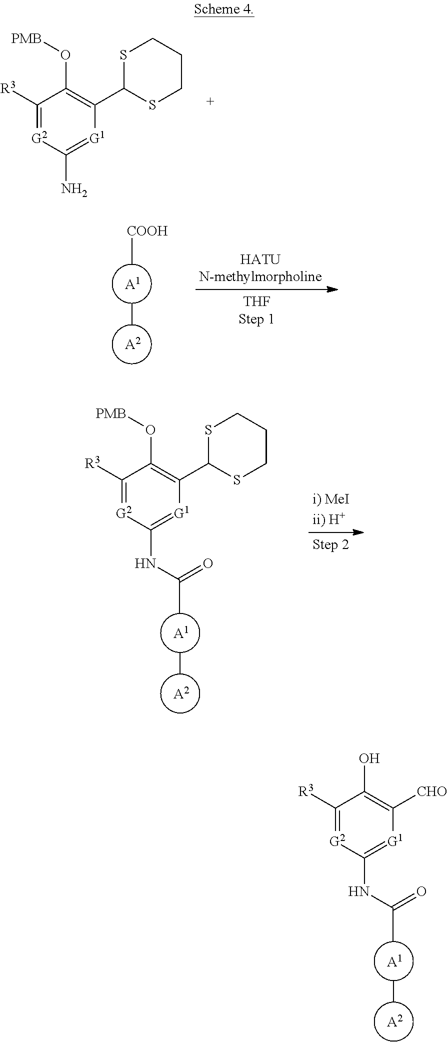

wherein R³, G¹, G², A¹, and A² are as defined for Formula (I), or any variation thereof detailed herein.

In some variations of the foregoing Scheme 4, compounds of Formula (I) may be synthesized according to Scheme 4-1.

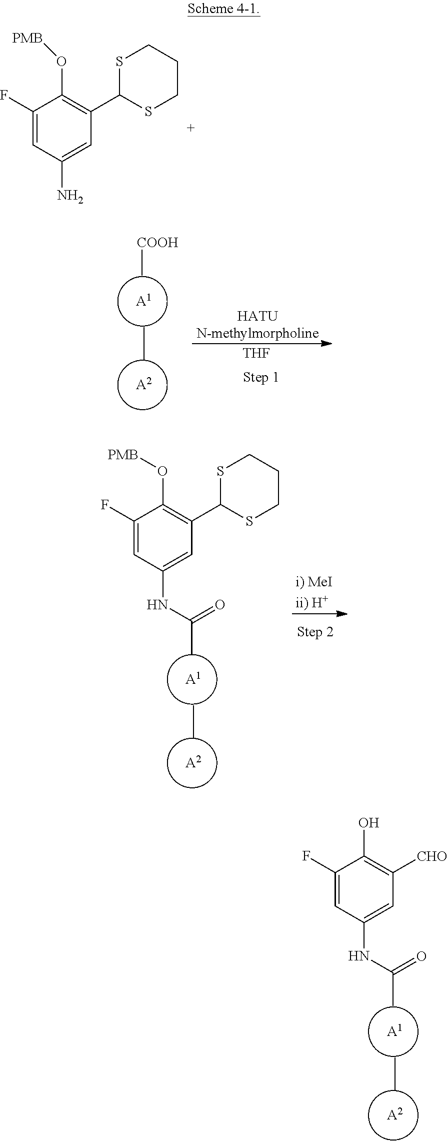

wherein A¹ is optionally substituted 5-membered heteroaryl and A² is optionally substituted 6-membered heterocyclyl or optionally substituted phenyl.

In some embodiments, compounds of Formula (I) may be synthesized according to Scheme 5.

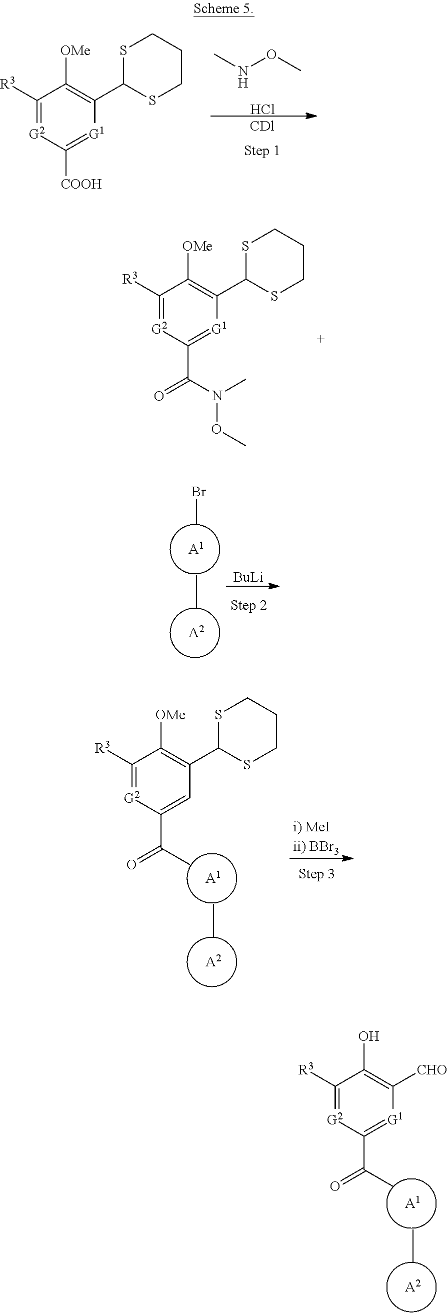

wherein R³, G¹, G², A¹, and A² are as defined for Formula (I), or any variation thereof detailed herein.

In some variations of the foregoing Scheme 5, compounds of Formula (I) may be synthesized according to Scheme 5-1.

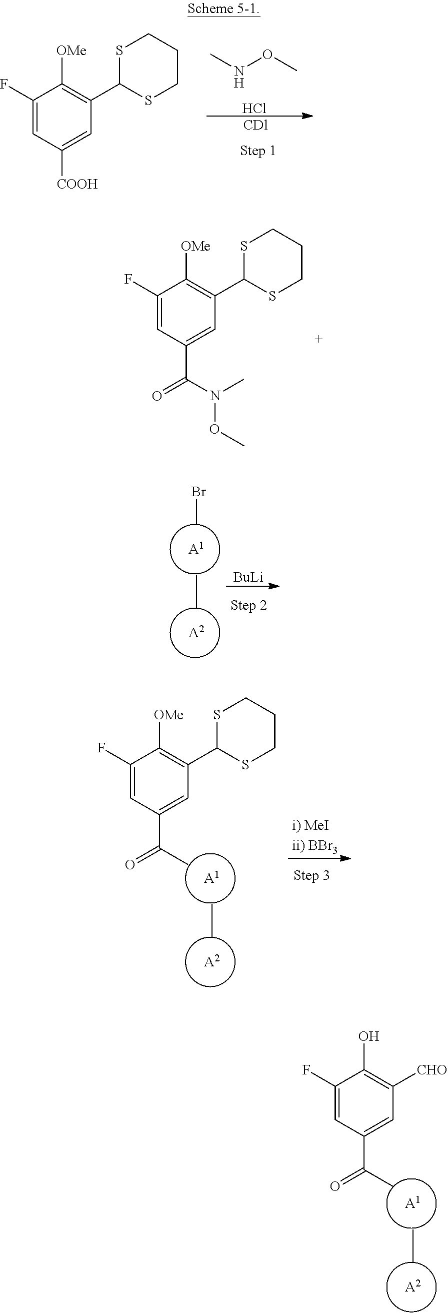

wherein A¹ is optionally substituted phenyl and A² is optionally substituted heterocyclyl.

In some embodiments, compounds of Formula (I) may be synthesized according to Scheme 6.

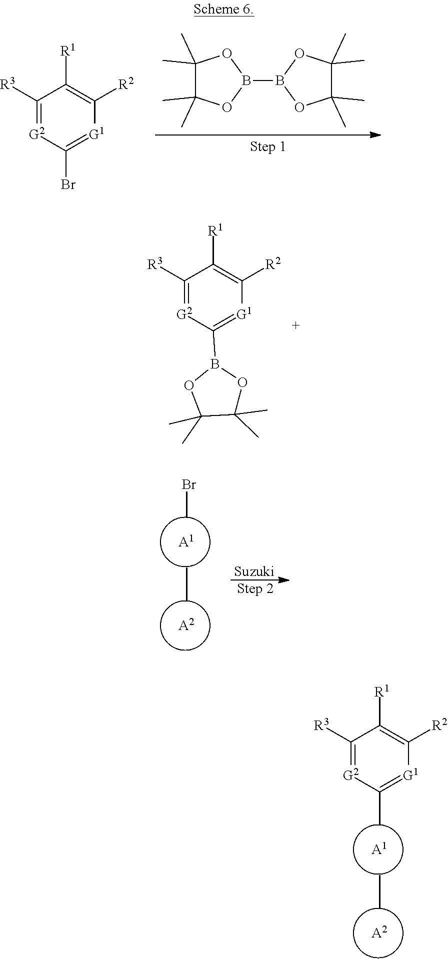

wherein R¹, R², R³, G¹, G², A¹, and A² are as defined for Formula (I), or any variation thereof detailed herein.

In some variations of the foregoing Scheme 6, compounds of Formula (I) may be synthesized according to Scheme 6-1.

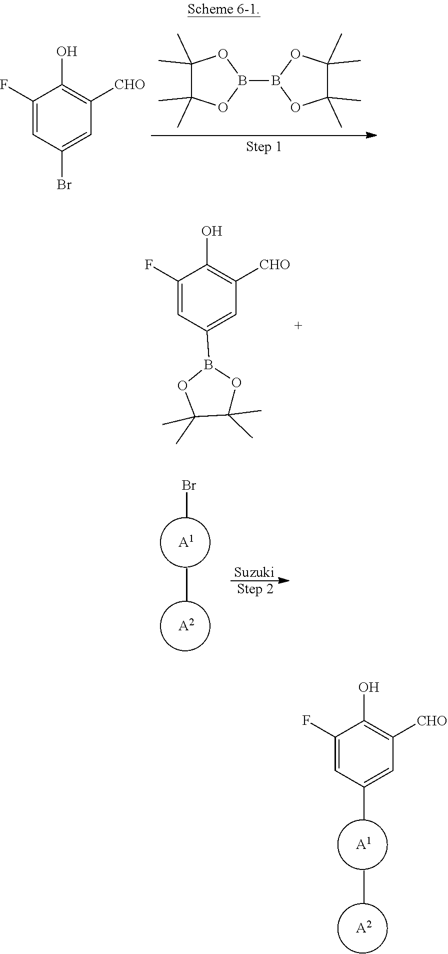

wherein A¹ is optionally substituted 5-membered heteroaryl and A² is optionally substituted 6-membered heterocyclyl or optionally substituted phenyl.

In some embodiments, compounds of Formula (I) may be synthesized according to Scheme 7a.

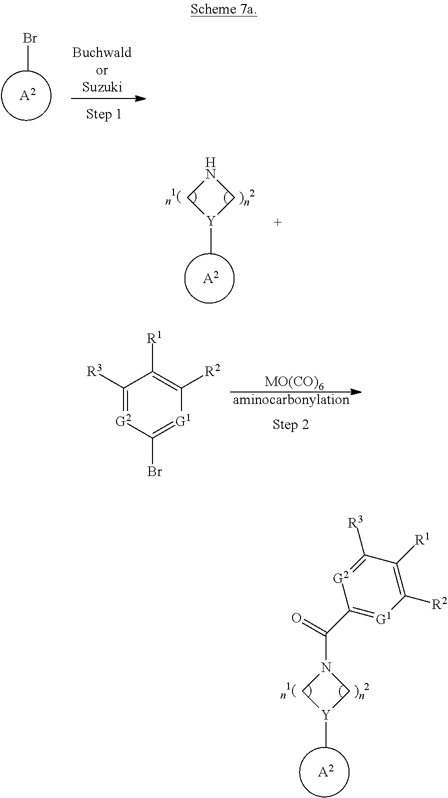

wherein R¹, R², R³, G¹, G², and A² are as defined for Formula (I), or any variation thereof detailed herein; Y¹ is CH or N; n¹ and n² are each independently 1 or 2.

In some variations of the foregoing Scheme 7a, compounds of Formula (I) may be synthesized according to Scheme 7a-1.

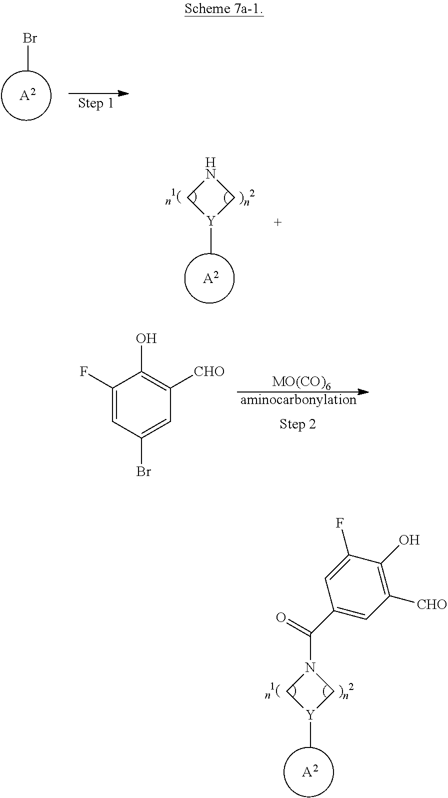

wherein A² is optionally substituted phenyl; Y¹ is CH or N; n¹ and n² are each independently 1 or 2.

In some embodiments, compounds of Formula (I) may be synthesized according to Scheme 7b.

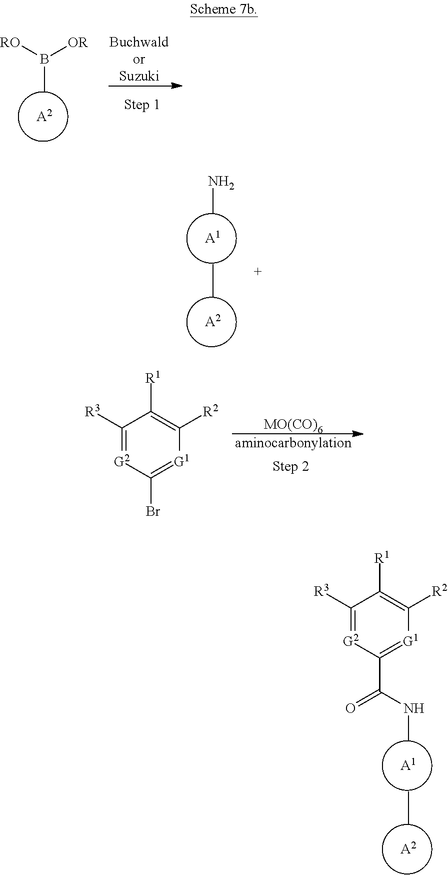

wherein R¹, R², R³, G¹, G², A¹ and A² are as defined for Formula (I), or any variation thereof detailed herein.

In some variations of the foregoing Scheme 7b, compounds of Formula (I) may be synthesized according to Scheme 7b-1.

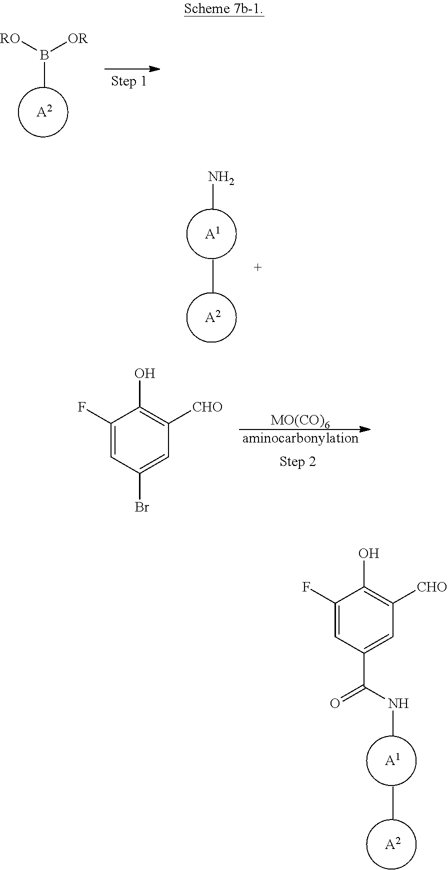

wherein A¹ and A² are as defined for Formula (I), or any variation thereof detailed herein.

In some embodiments, compounds of Formula (I) may be synthesized according to Scheme 8.

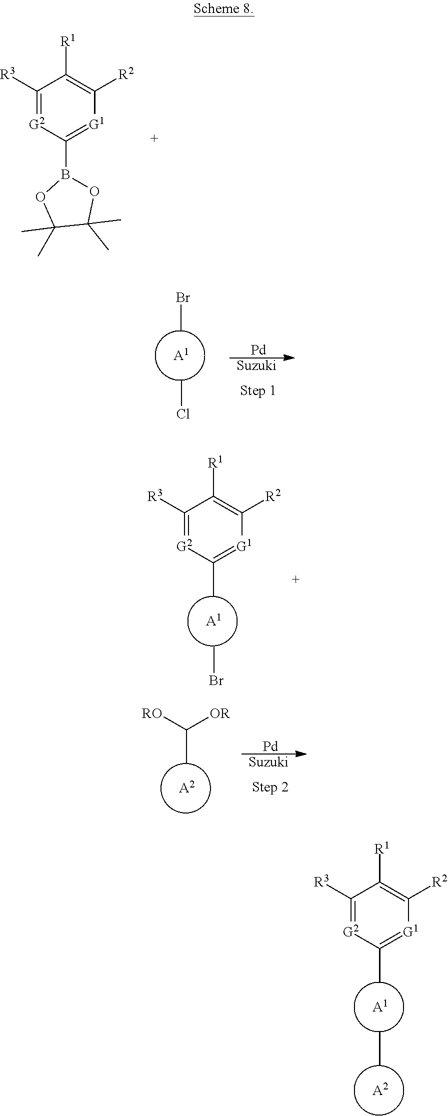

wherein R¹, R², R³, G¹, G², A¹, and A² are as defined for Formula (I), or any variation thereof detailed herein.

In some variations of the foregoing Scheme 8, compounds of Formula (I) may be synthesized according to Scheme 8-1.

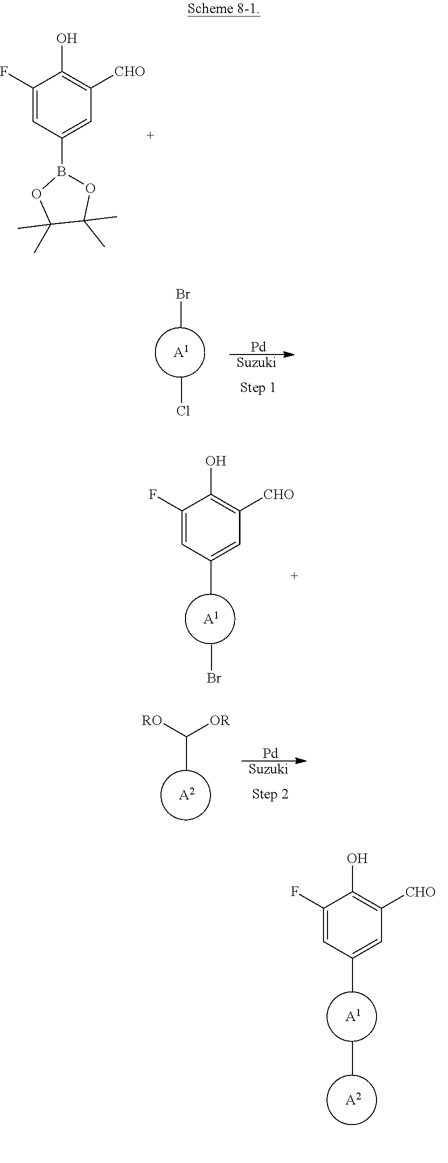

wherein A¹ is optionally substituted 5-membered heteroaryl; and A² is optionally substituted 6-membered heterocyclyl or optionally substituted phenyl.

In some embodiments, compounds of Formula (I) may be synthesized according to Scheme 9.

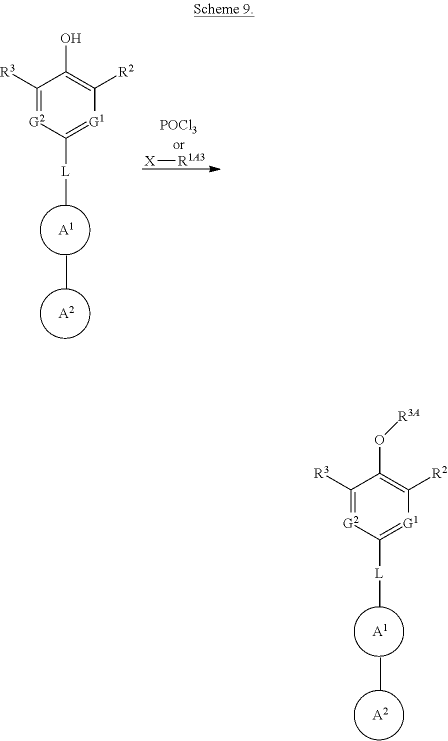

wherein R², R³, G¹, G², L, A¹, and A² are as defined for Formula (I), or any variation thereof detailed herein; X is fluoro, bromo, chloro, —SMe, or trifluoromethanesulfonate (or —OTf); and R^{1A3} is optionally substituted heteroaryl, —PO₃H₂, —P(O)H(OC₁₋₆ alkyl), or —P(O)(OC₁₋₆ alkyl)(OC₁₋₆ alkyl).

In some embodiments of the foregoing Scheme 9, compounds of Formula (I) may be synthesized according to Scheme 9-1.

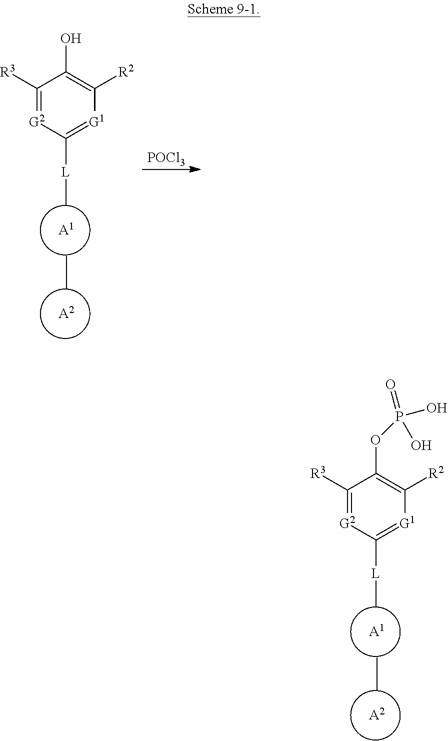

wherein R², R³, G¹, G², L, A¹, and A² are as defined for Formula (I), or any variation thereof detailed herein.

In some variations of the foregoing Scheme 9, compounds of Formula (I) may be synthesized according to Scheme 9-2.

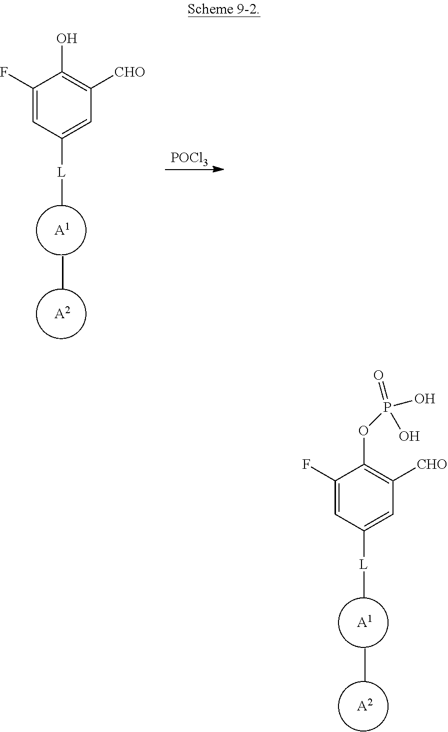

wherein L is as defined for Formula (I), or any variation thereof detailed herein; A¹ is optionally substituted 5-membered heteroaryl; and A² is optionally substituted 6-membered heterocyclyl or phenyl.

In some embodiments of the foregoing Scheme 9, compounds of Formula (I) may be synthesized according to Scheme 9-3.

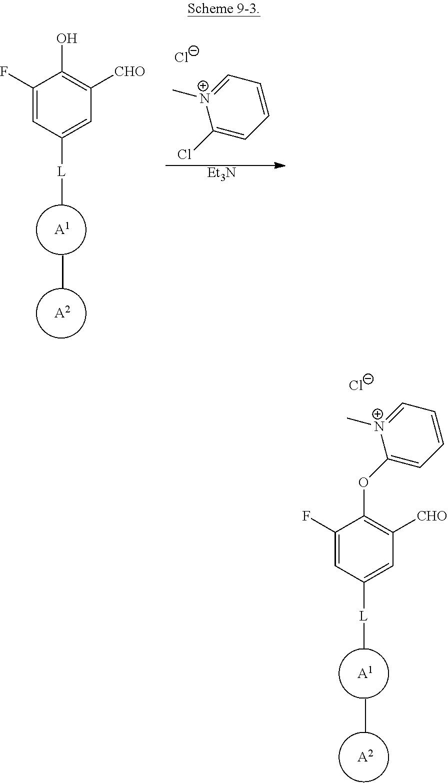

wherein L, A¹, and A² are as defined for Formula (I), or any variation thereof detailed herein.

In some embodiments, compounds of Formula (I) may be synthesized according to Scheme 10.

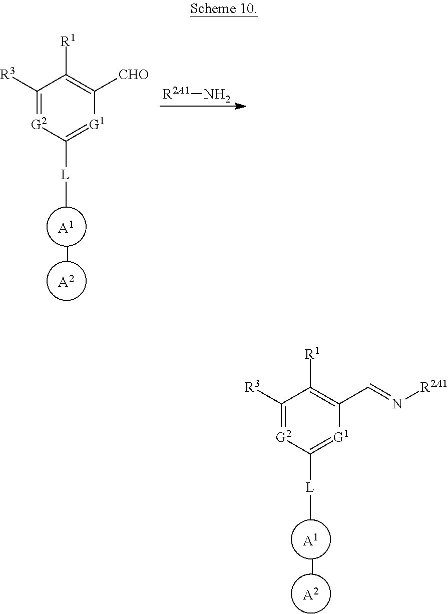

wherein R¹, R³, G¹, G², L, A¹, A², and R^{2A1} are as defined for Formula (I), or any variation thereof detailed herein.

In some variations of the foregoing Scheme 10, compounds of Formula (I) may be synthesized according to Scheme 10-1.

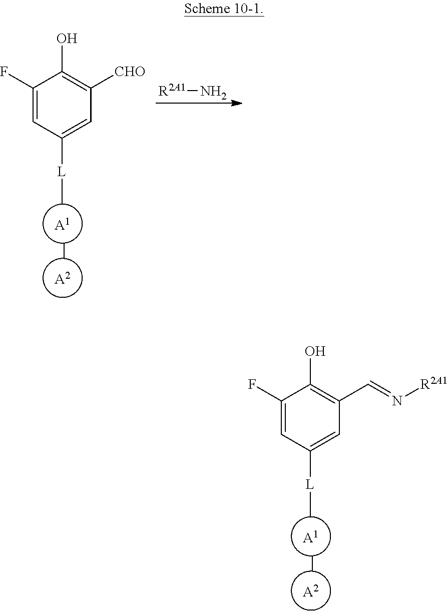

wherein L, A¹, A², and R^{2A1} are as defined for Formula (I), or any variation thereof detailed herein.

In some embodiments, compounds of Formula (I) may be synthesized according to Scheme 11.

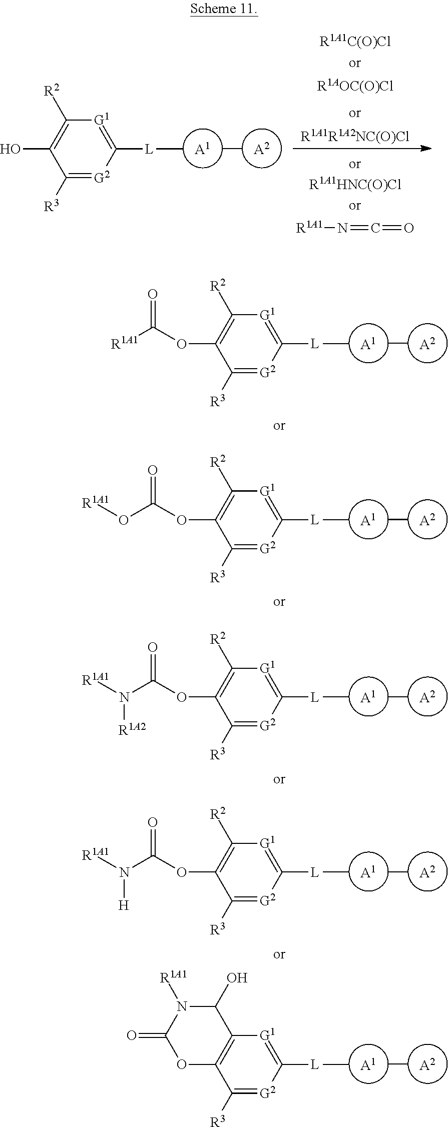

wherein R², R³, G¹, G², L, A¹, A², R^{1A1}, and R^{1A2} are as defined for Formula (I), or any variation thereof detailed herein.

In some variations of the foregoing Scheme 11, compounds of Formula (I) may be synthesized according to Scheme 11-1.

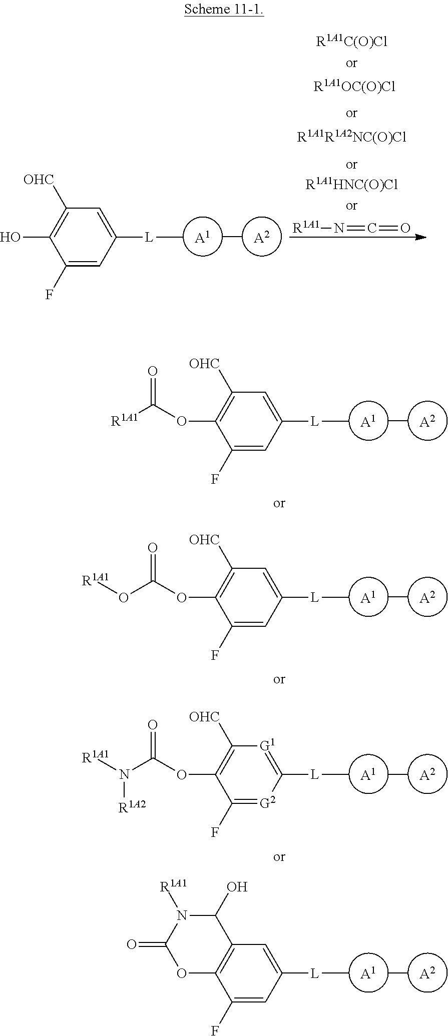

wherein L, A¹, A², R^{1A}1, and R^{1A2} are as defined for Formula (I), or any variation thereof detailed herein.

In some embodiments, compounds of Formula (I) may be synthesized according to Scheme 12.

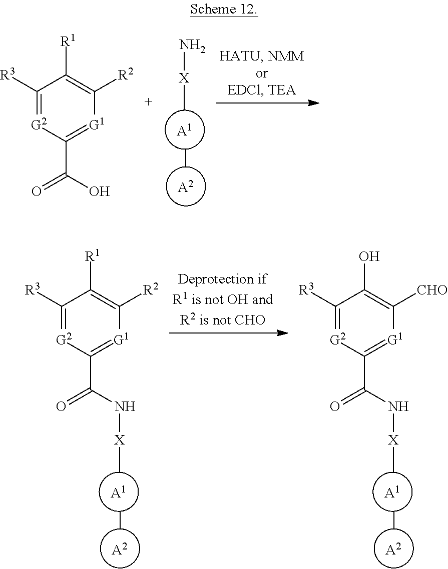

wherein R³, A¹, A², G¹, and G² are as defined for Formula (I), or any variation thereof detailed herein; R¹ is —OH, —O(p-methoxybenzyl) (same as —OPMB), —O(benzyl) (same as —OBn), or —O(methyl) (same as —OMe); R² is —CHO or

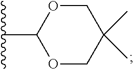 [SMILES: *C1OCC(C)(C)CO1]

and X is a bond, —NH—, —NHC(O)—*, or —(CH₂)₁₋₃—.

In some variations of the foregoing Scheme 12, compounds of Formula (I) may be synthesized according to Scheme 12-1.

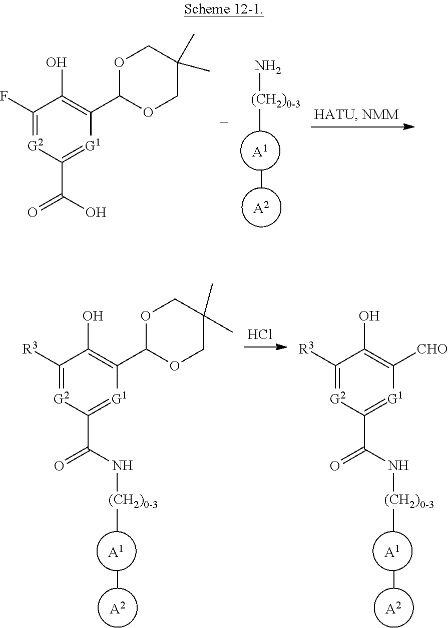

wherein G¹, G², A¹, A², and R³ are as defined for Formula (I), or any variation thereof detailed herein.

In some variations of the foregoing Scheme 12, compounds of Formula (I) may be synthesized according to Scheme 12-2.

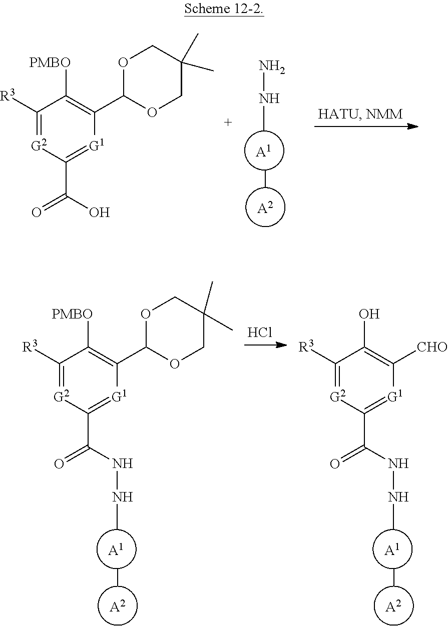

wherein G¹, G², A¹, A², and R³ are as defined for Formula (I), or any variation thereof detailed herein.

In some variations of the foregoing Scheme 12, compounds of Formula (I) may be synthesized according to Scheme 12-3.

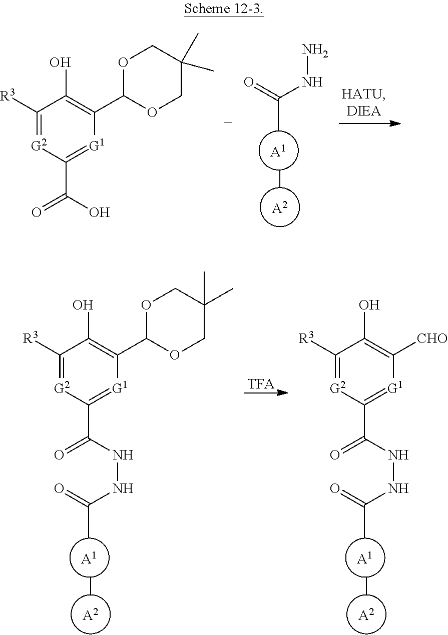

wherein G¹, G², A¹, A², and R³ are as defined for Formula (I), or any variation thereof detailed herein.

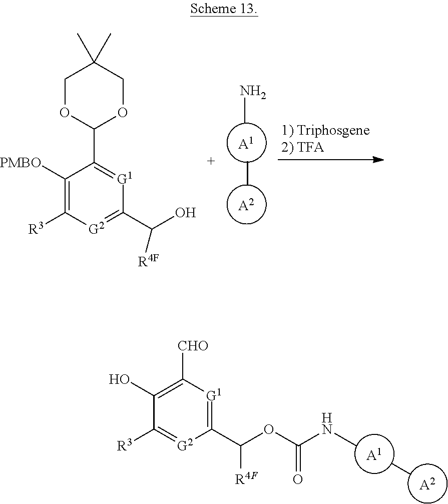

wherein R³, A¹, A², G¹, and G² are as defined for Formula (I), or any variation thereof detailed herein; and R^{4F} is H, C₁₋₆ alkyl, or C₁₋₆ haloalkyl.

In some embodiments, compounds of Formula (I) may be synthesized according to Scheme 14.

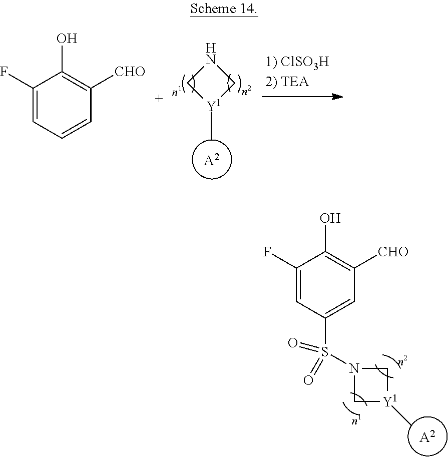

wherein A² is as defined for Formula (I), or any variation thereof detailed herein; and n¹ and n² are each independently 0, 1, 2, or 3; and Y¹ is CH or N.

In some embodiments, compounds of Formula (I) may be synthesized according to Scheme 15.

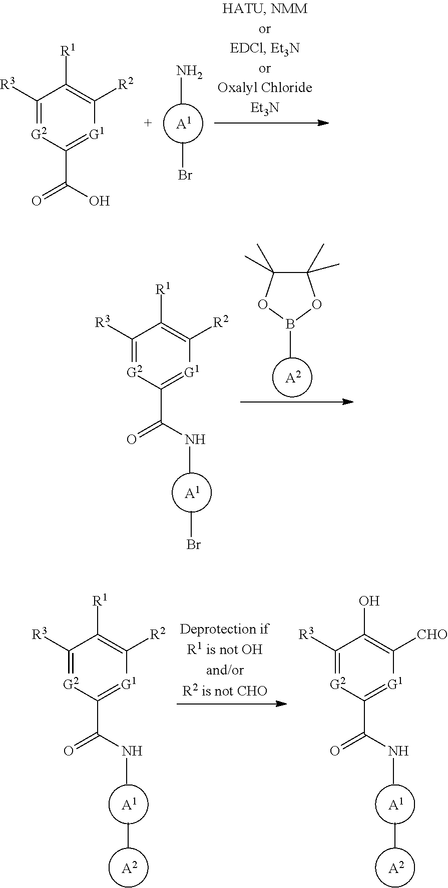

wherein R¹, R², R³, G¹, G², A¹, and A², are as defined for Formula (I), or any variation thereof detailed herein; and n¹ and n² are each independently 0, 1, 2, or 3. In some variations, R¹ is —OH or —OBn. In some variations, R² is —CHO or

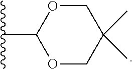 [SMILES: *C1OCC(C)(C)CO1]

In some variations of the foregoing Scheme 15, compounds of Formula (I) may be synthesized according to Scheme 15-1.

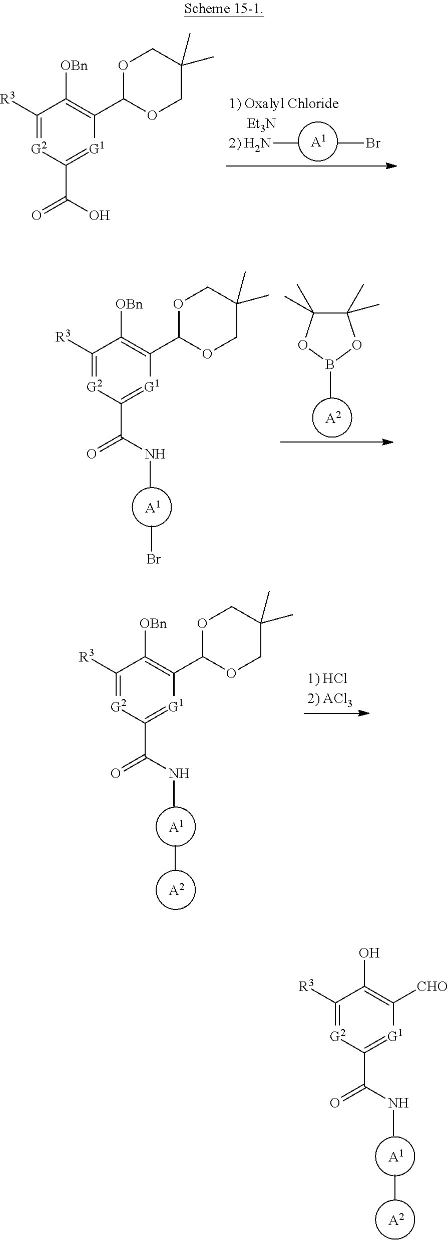

wherein R³, G¹, G², A¹, and A² are as defined for Formula (I), or any variation thereof detailed herein.

In some embodiments, compounds of Formula (I) may be synthesized according to Scheme 16.

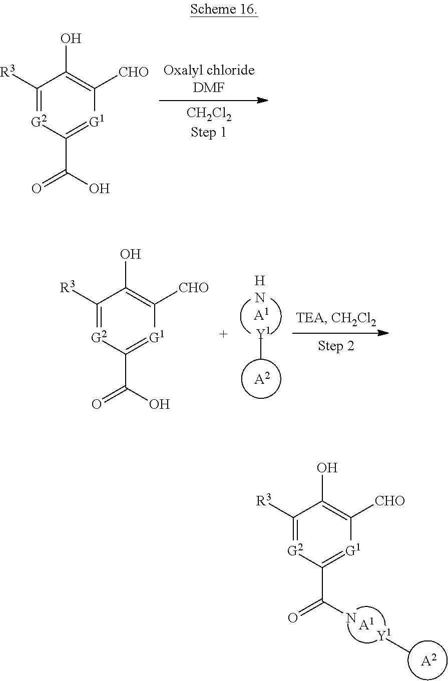

wherein R³, G¹, G², and A², are as defined for Formula (I), or any variation thereof detailed herein; A¹ is optionally substituted heterocyclyl containing N and Y¹; and Y¹ is CH or N. In some variations, Y¹ is CH.

In some embodiments, compounds of Formula (I) may be synthesized according to Scheme 17.

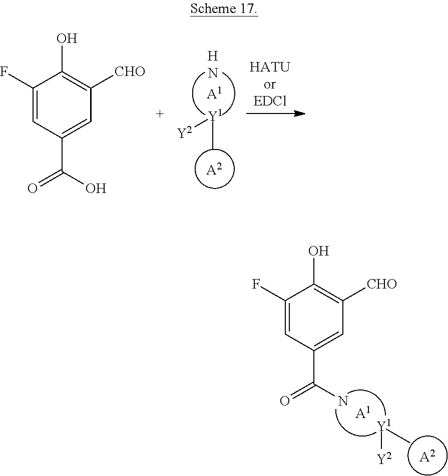

wherein A² is as defined for Formula (I), or any variation thereof detailed herein; A¹ is optionally substituted heterocyclyl containing N and Y¹; Y¹ is C or N; and Y² is H or —OH.

Chemical Synthesis

Exemplary chemical entities useful in methods of the present disclosure will now be described by reference to the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Furthermore, one of skill in the art will recognize that the transformations shown in the schemes below may be performed in any order that is compatible with the functionality of the particular pendant groups. Each of the reactions depicted in the general schemes may be run at a temperature from about 0° C. to the reflux temperature of the organic solvent used. Isotopically labeled compounds as described herein are prepared according to the methods described below, using suitably labeled starting materials. Such materials are generally available from commercial suppliers of radiolabeled chemical reagents.

### EXAMPLES

The following examples are offered to illustrate but not to limit the present disclosure. One of skill in the art will recognize that the following synthetic reactions and schemes may be modified by choice of suitable starting materials and reagents in order to access other compounds of Formula (I). The compounds are prepared using the general methods described above.

The following abbreviations are used throughout the Examples: ACN or MeCN (acetonitrile), AIBN (azobisisobutyronitrile), BINAP (2,2′-bis(diphenylphosphino)-1,1′-binaphthyl), BuLi (butyl lithium), BuOH (butanol), DBU (1,8-diazabicyclo[5.4.0]undec-7-ene), DCM (dichloromethane), DIEA or DIPEA (N,N-diisopropylethylamine), DMF (N,N-dimethylformamide), DMSO (dimethyl sulfoxide), DPPA (diphenylphosphoryl azide), EA or EtOAc (Ethyl acetate), EDCl (1-Ethyl-3-(3-dimethylaminopropyl)carbodiimide), HATU (1-[Bis(dimethylamino)methylene]-1H-1,2,3-triazolo[4,5-b]pyridinium 3-oxide hexafluorophosphate), MeOH (methanol), MsCl (methanesulfonyl chloride), NBS (N-bromosuccinimide), NMM (N-methylmorpholine), PdCl₂(dppf) ((1,1′-bis(diphenylphosphino)ferrocene))palladium(II) dichloride), dppf (1,1′-bis(diphenylphosphino)ferrocene), Pd(PPh₃)₂Cl₂ (bis(triphenylphosphine)palladium(II) dichloride), Pd(PPh₃)₄(tetrakis(triphenylphosphine)palladium(0)), PE (petroleum ether), PMB (4-methoxybenzyl), PPh₃ (triphenylphosphane), rt (room temperature), Ruphos (2-Dicyclohexylphosphino-2′,6′-diisopropoxybiphenyl), RuPhos Pd G3 ((2-Dicyclohexylphosphino-2′,6′-diisopropoxy-1,1′-biphenyl)[2-(2′-amino-1,1′-biphenyl)]palladium(II) methanesulfonate), TBAF (tetrabutylammonium fluoride), TEA (triethylamine), TFA (trifluoroacetic acid), THF (tetrahydrofuran), TMS-diazomethane (tetramethylsilyldiazomethane), TLC (thin layer chromatography), XantPhos (4,5-Bis(diphenylphosphino)-9,9-dimethylxanthene), XantPhos Pd G3 ([(4,5-Bis(diphenylphosphino)-9,9-dimethylxanthene)-2-(2′-amino-1,1′-biphenyl)]palladium(II) methanesulfonate), Xphos (2-Dicyclohexylphosphino-2′,4′,6′-triisopropylbiphenyl), and XPhos Pd G3 ((2-Dicyclohexylphosphino-2′,4′,6′-triisopropyl-1,1′-biphenyl)[2-(2′-amino-1,1′-biphenyl)]palladium(II) methanesulfonate).

### Example 1: 3-fluoro-5-formyl-4-hydroxy-N-(5-(piperidin-1-yl)-1,3,4-thiadiazol-2-yl)benzamide (Compound 1)

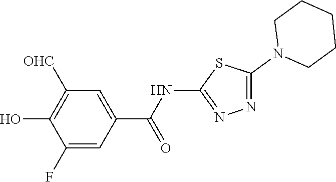 [SMILES: O=Cc1cc(C(=O)Nc2nnc(N3CCCCC3)s2)cc(F)c1O]

In a 50 mL glass vial, oxalyl chloride (256 mg, 2.02 mmol, 2.0 eq.) and DMF (1 drop) were added to a solution of 3-fluoro-5-formyl-4-methoxybenzoic acid (200 mg, 1.01 mmol, 1.0 eq.) in CH₂Cl₂ (10 mL). The reaction was stirred for 1 hour at room temperature. The solvent was removed in vacuo and the residue was co-evaporated with CH₂Cl₂ for two times. The residue was then dissolved in CH₂Cl₂ (10 mL). 5-(Piperidin-1-yl)-1,3,4-thiadiazol-2-amine (280 mg, 1.52 mmol, 1.5 eq.) and TEA (306 mg, 3.03 mmol, 3.0 eq.) were added. The reaction was stirred overnight at room temperature. The mixture was poured into water and extracted with CH₂Cl₂ for three times. The organic extracts were combined, washed with brine, dried over anhydrous sodium sulfate, and concentrated. The residue was purified by silica gel column chromatography to give intermediate (158 mg, 43% yield) as a yellow solid. The intermediate (158 mg, 0.43 mmol, 1.0 eq.) was dissolved in CH₂Cl₂ (5 mL) and cooled to −78° C. BBr₃ (1.07 g, 4.3 mmol, 10 eq.) was added dropwise. The reaction was stirred overnight at room temperature. The reaction mixture was poured into ice-cold sat. sodium bicarbonate and extracted with ethyl acetate for three times. The organic extracts were combined, washed with brine, dried over anhydrous sodium sulfate, and concentrated. The residue was purified by silica gel column chromatography and prep-TLC to give 3-fluoro-5-formyl-4-hydroxy-N-(5-(piperidin-1-yl)-1,3,4-thiadiazol-2-yl)benzamide (33 mg, 0.09 mmol, 9% yield) as an off-white solid. ¹H NMR (DMSO-d6, 400 MHz) δ: 12.00 (br, 1H), 10.30 (s, 1H), 8.25 (d, J=1.6 Hz, 1H), 8.10 (dd, J=11.6 Hz, 1.6 Hz, 1H), 3.41 (m, 4H), 1.60 (m, 6H). LC-MS m/z [M+H]⁺ calc'd for C₁₅H₁₅FN₄O₃S, 351; found, 351.

### Example 2: 3-fluoro-5-formyl-4-hydroxy-N-(5-phenyl-1,3,4-thiadiazol-2-yl)benzamide (Compound 2)

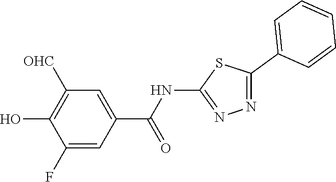 [SMILES: O=Cc1cc(C(=O)Nc2nnc(-c3ccccc3)s2)cc(F)c1O]

The title compound was prepared from 3-fluoro-5-formyl-4-methoxybenzoic acid (200 mg, 1.01 mmol, 1.0 eq.) and 5-phenyl-1,3,4-thiadiazol-2-amine (268 mg, 1.52 mmol, 1.5 eq.) using a method similar to that as described in Example 1 to give the title compound (70 mg, 0.20 mmol, 20% yield) as a light yellow solid. ¹H NMR (DMSO-d6, 400 MHz) δ: 12.96 (br, 1H), 10.28 (s, 1H), 8.29 (d, J=2.0 Hz, 1H), 8.05 (dd, J=12.4 Hz, 2.0 Hz, 1H), 7.98 (m, 2H), 7.55 (m, 3H). LC-MS m/z [M+H]⁺ calc'd for C₁₆H₁₀FN₃O₃S, 344; found, 344.

### Example 3: 3-fluoro-5-formyl-4-hydroxy-N-(5-(piperidin-1-yl)thiazol-2-yl)benzamide (Compound 3)

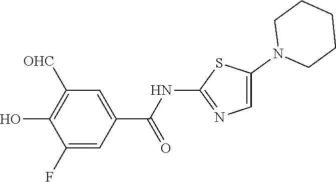 [SMILES: O=Cc1cc(C(=O)Nc2ncc(N3CCCCC3)s2)cc(F)c1O]

The title compound was prepared from 3-fluoro-5-formyl-4-methoxybenzoic acid (200 mg, 1.01 mmol, 1.0 eq.) and 5-(Piperidin-1-yl)thiazol-2-amine (277 mg, 1.52 mmol, 1.5 eq.) using a method similar to that as described in Example 1 to give the title compound (8 mg, 0.02 mmol, 2% yield) as a yellow solid. ¹H NMR (DMSO-d6, 400 MHz) δ: 12.26 (br, 1H), 10.31 (s, 1H), 8.26 (d, J=2.0 Hz, 1H), 8.16 (dd, J=13.6 Hz, 2.0 Hz, 1H), 6.68 (s, 1H), 3.02 (m, 4H), 1.62 (m, 4H), 1.52 (m, 2H). LC-MS m/z [M+H]⁺ calc'd for C₁₆H₁₆FN₃O₃S, 350; found, 350.

### Example 4: 3-fluoro-5-formyl-4-hydroxy-N-(5-phenylthiazol-2-yl)benzamide (Compound 4)

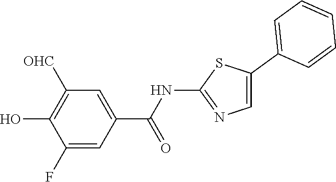 [SMILES: O=Cc1cc(C(=O)Nc2ncc(-c3ccccc3)s2)cc(F)c1O]

The title compound was prepared from 3-fluoro-5-formyl-4-methoxybenzoic acid (200 mg, 1.01 mmol, 1.0 eq.) and 5-Phenylthiazol-2-amine (268 mg, 1.52 mmol, 1.5 eq.) using a method similar to that as described in Example 1 to give the title compound (38 mg, 0.11 mmol, 11% yield) as a light yellow solid. ¹H NMR (DMSO-d6, 400 MHz) δ: 12.82 (br, 1H), 11.86 (br, 1H), 10.34 (s, 1H), 8.35 (d, J=1.6 Hz, 1H), 8.25 (dd, J=13.2 Hz, 1.6 Hz, 1H), 7.99 (s, 1H), 7.66 (d, J=8.4 Hz, 2H), 7.43 (m, 2H), 7.31 (m, 1H). LC-MS m/z [M+H]⁺ calc'd for C₁₇H₁₁FN₂O₃S, 343; found, 343.

### Example 5: N-(5-(4-cyclopropylpiperazin-1-yl)thiazol-2-yl)-3-fluoro-5-formyl-4-hydroxybenzamide (Compound 5)

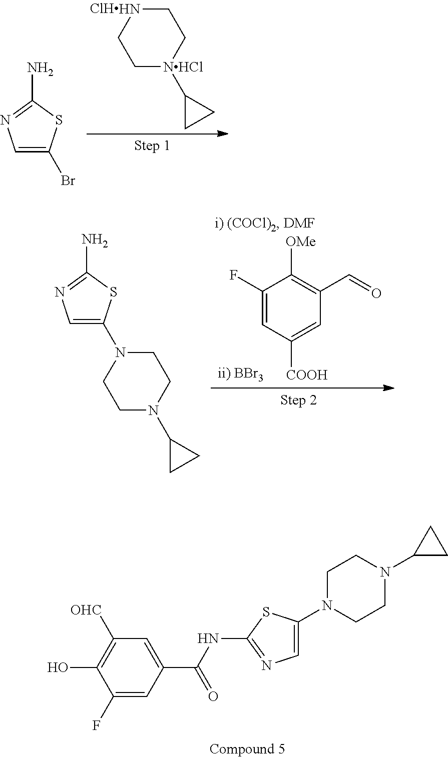

Step 1: Synthesis of 5-(4-cyclopropylpiperazin-1-yl)thiazol-2-amine: In a 50 mL glass vial, K₂CO₃ (2.56 g, 18.6 mmol, 3.0 eq.) was added to a mixture of 5-bromothiazol-2-amine (1.34 g, 7.4 mmol, 1.2 eq.) and 1-cyclopropylpiperazine dihydrochloride (1.2 g, 6.2 mmol, 1.0 eq.) in DMF (10 mL). The reaction was stirred for 2 hours at 80° C. The reaction mixture was poured into water (100 mL) and extracted with ethyl acetate for three times. The organic extracts were combined, washed with brine, dried over anhydrous sodium sulfate, and concentrated. The residue was purified by silica gel column chromatography (CH₂Cl₂/MeOH=200:1 to 150:1) to give 5-(4-cyclopropylpiperazin-1-yl)thiazol-2-amine (590 mg, 2.63 mmol, 42% yield). ¹H NMR (CDCl3, 400 MHz) δ: 6.44 (s, 1H), 4.64 (br, 2H), 2.93 (t, J=4.8 Hz, 4H), 2.74 (t, J=5.0 Hz, 4H), 1.66 (m, 1H), 0.47 (m, 2H), 0.44 (m, 2H).

Step 2: The title compound was prepared from 3-fluoro-5-formyl-4-methoxybenzoic acid (410 mg, 2.07 mmol, 1.0 eq.) and 5-(4-Cyclopropylpiperazin-1-yl)thiazol-2-amine (510 mg, 2.28 mmol, 1.1 eq.) using a method similar to that as described in Example 1 to give the final title compound (16 mg, 0.04 mmol, 14% yield) as a yellow solid. ¹H NMR (DMSO-d6, 400 MHz) δ: 12.22 (br, 1H), 10.30 (s, 1H), 8.23 (d, J=1.6 Hz, 1H), 8.11 (dd, J=12.0 Hz, 2.0 Hz, 1H), 6.70 (s, 1H), 3.00 (m, 4H), 2.71 (m, 4H), 1.74 (m, 1H), 0.47 (m, 2H), 0.38 (m, 2H). LC-MS m/z [M+H]⁺ calc'd for C₁₈H₁₉FN₄O₃S, 391; found, 391.

### Example 6: 3-fluoro-5-formyl-4-hydroxy-N-(4-phenylthiazol-2-yl)benzamide (Compound 6)

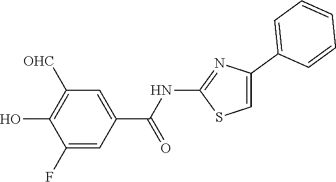 [SMILES: O=Cc1cc(C(=O)Nc2nc(-c3ccccc3)cs2)cc(F)c1O]

In a 50 mL glass vial, POCl₃ (840 mg, 5.44 mmol, 2.0 eq.) was added dropwise to a solution of 3-fluoro-5-formyl-4-hydroxybenzoic acid (500 mg, 2.72 mmol, 1.0 eq.) and 4-phenylthiazol-2-amine (574 mg, 3.26 mmol, 1.2 eq.) in pyridine (10 mL) at 0° C. The reaction was then stirred overnight at room temperature. The solution was poured into water and pH was adjusted to 4-5 with 5% KHSO₄. The resulting mixture was then extracted with ethyl acetate for three times. The organic extracts were combined, washed with brine, dried over anhydrous sodium sulfate, and concentrated. The residue was purified by silica gel column chromatography and prep-TLC for several times to give 3-fluoro-5-formyl-4-hydroxy-N-(4-phenylthiazol-2-yl)benzamide (7 mg, 0.02 mmol, 0.8% yield) as a yellow solid. ¹H NMR (DMSO-d6, 400 MHz) δ: 12.86 (br, 1H), 11.86 (br, 1H), 10.34 (s, 1H), 8.37 (s, 1H), 8.27 (dd, J=11.6 Hz, 2.0 Hz, 1H), 7.96 (d, J=7.2 Hz, 2H), 7.71 (s, 1H), 7.45 (m, 2H), 7.34 (m, 1H). LC-MS m/z [M+H]⁺ calc'd for C₁₇H₁₁FN₂O₃S, 343; found, 343.

### Example 7: 3-fluoro-5-formyl-4-hydroxy-N-(2-(piperidin-1-yl)thiazol-5-yl)benzamide (Compound 7)

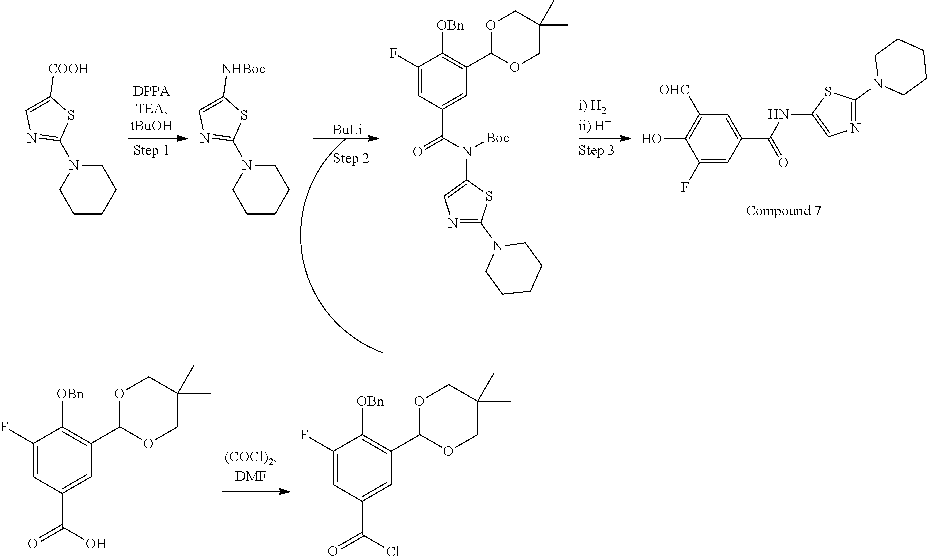

Step 1: Synthesis of tert-butyl 2-(piperidin-1-yl)thiazol-5-ylcarbamate: In a 100 mL glass vial, a solution of 2-(piperidin-1-yl)thiazole-5-carboxylic acid (2.7 g, 12.7 mmol, 1.0 eq.), DPPA (4.54 g, 16.5 mmol, 1.3 eq.), and TEA (2.05 g, 20.32 mmol, 1.6 eq.) in tBuOH (30 mL) was heated for 6 h at 85° C. The solvent was removed in vacuo and the residue was dissolved in water/EtOAc. The mixture was then extracted with ethyl acetate for three times. The organic extracts were combined, washed with sat. sodium bicarbonate and brine, dried over anhydrous sodium sulfate, and concentrated. The residue was purified by silica gel column chromatography (petroleum ether/EtOAc/CH₂Cl₂=20:1:1 to 5:1:1) to give tert-butyl 2-(piperidin-1-yl)thiazol-5-ylcarbamate (2.47 g, 8.73 mmol, 69% yield) as a brown powder. LC-MS m/z [M+H]⁺ calc'd for C₁₃H₂₁N₃O₂S, 284; found, 284.

Step 2: Synthesis of tert-butyl 4-(benzyloxy)-3-(5,5-dimethyl-1,3-dioxan-2-yl)-5-fluorobenzoyl(2-(piperidin-1-yl)thiazol-5-yl)carbamate: In a 50 mL glass vial, oxalyl chloride (282 mg, 2.22 mmol, 2.0 eq.) was added to a solution of 4-(benzyloxy)-3-(5,5-dimethyl-1,3-dioxan-2-yl)-5-fluorobenzoic acid (400 mg, 1.11 mmol, 1.0 eq.) in CH₂Cl₂ (10 mL). A drop of DMF was added and the reaction was stirred for 2 hours at room temperature. The solvent was removed in vacuo and the residue was co-evaporated with CH₂Cl₂ for two times. In a 100 mL three-neck glass bottle, tert-butyl 2-(piperidin-1-yl)thiazol-5-ylcarbamate (220 mg, 0.78 mmol, 0.7 eq.) was dissolved in THF (10 mL) under nitrogen protection. The solution was cooled to −78° C. and BuLi (0.98 mL, 1.6 M in THF/hexane, 1.56 mmol, 1.4 eq.) was added. The reaction was stirred for 1.5 hours at −40° C. Then a solution of the intermediate in vial 1 in THF (5 mL) was added dropwise. The reaction was then stirred overnight at room temperature. The mixture was poured into sat. NH₄Cl and extracted with ethyl acetate for three times. The organic extracts were combined, washed with brine, dried over anhydrous sodium sulfate, and concentrated. The residue was purified by silica gel column chromatography (Petroleum ether/EtOAc=100:1 to 30:1) to give tert-butyl 4-(benzyloxy)-3-(5,5-dimethyl-1,3-dioxan-2-yl)-5-fluorobenzoyl(2-(piperidin-1-yl)thiazol-5-yl)carbamate (190 mg, 39% yield) as a yellow oil. LC-MS m/z [M+H]⁺ calc'd for C₃₃H₄₀FN₃O₆S, 626; found, 626.

Step 3: In a 100 mL glass vial, a mixture of tert-butyl 4-(benzyloxy)-3-(5,5-dimethyl-1,3-dioxan-2-yl)-5-fluorobenzoyl(2-(piperidin-1-yl)thiazol-5-yl)carbamate (190 mg, 0.30 mmol, 1.0 eq.) and Pd/C (20 mg) in MeOH (10 mL) was hydrogenated for 2 hours at room temperature. Pd/C was filtered off and the filtrate was concentrated in vacuo. The residue was purified by prep-TLC to give intermediate (62 mg, 0.12 mmol, 39% yield) as a yellow powder. The intermediate (62 mg, 0.12 mmol) was dissolved in THF (5 mL) and 4 N HCl (5 mL) was added. The reaction was stirred overnight at room temperature. The mixture was extracted with ethyl acetate for three times. The organic extracts were combined, washed with brine, dried over anhydrous sodium sulfate, and concentrated. The residue was purified by prep-TLC to give 3-fluoro-5-formyl-4-hydroxy-N-(2-(piperidin-1-yl)thiazol-5-yl)benzamide (30 mg, 0.09 mmol, 74% yield) as a yellow solid. LC-MS m/z [M+H]⁺ calc'd for C₁₆H₁₆FN₃O₃S, 350; found, 350.

### Example 8: 3-fluoro-5-formyl-4-hydroxy-N-(2-(pyrrolidin-1-yl)thiazol-5-yl)benzamide (Compound 8)

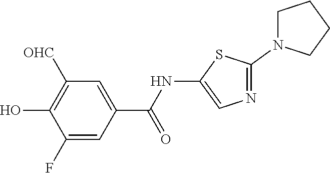 [SMILES: O=Cc1cc(C(=O)Nc2cnc(N3CCCC3)s2)cc(F)c1O]

The title compound was prepared from tert-butyl 4-(benzyloxy)-3-(5,5-dimethyl-1,3-dioxan-2-yl)-5-fluorobenzoyl(2-(pyrrolidin-1-yl)thiazol-5-yl)carbamate (0.93 g, 1.52 mmol, 1.0 eq.) (prepared as in Example 7) using a method similar to that as described in Example 7 to give the final title compound (45 mg, 0.13 mmol, 28% yield) as a yellow solid. ¹H NMR (DMSO-d6, 400 MHz) δ: 11.16 (br, 1H), 10.33 (s, 1H), 8.16 (d, J=1.6 Hz, 1H), 8.01 (dd, J=12.0 Hz, 2.0 Hz, 1H), 7.06 (s, 1H), 3.34 (m, 4H), 1.97 (m, 4H). LC-MS m/z [M+H]⁺ calc'd for C₁₅H₁₄FN₃O₃S, 336; found, 336.

### Example 9: N-(3-fluoro-5-formyl-4-hydroxyphenyl)-2-(piperidin-1-yl)thiazole-4-carboxamide (Compound 9)

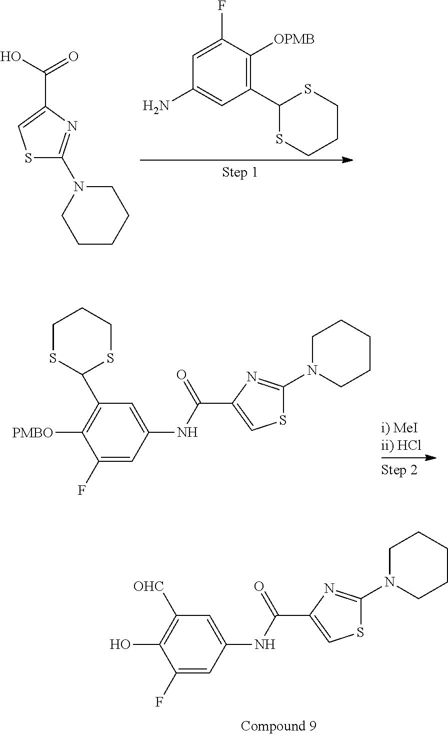

Step 1: Synthesis of N-(3-(1,3-dithian-2-yl)-5-fluoro-4-(4-methoxybenzyloxy)phenyl)-2-(piperidin-1-yl)thiazole-4-carboxamide: In a 100 mL glass vial, HATU (1.04 g, 2.74 mmol, 2.0 eq.) and N-methyl morpholine (553 mg, 5.48 mmol, 4.0 eq.) were added to a mixture of 3-(1,3-dithian-2-yl)-5-fluoro-4-(4-methoxybenzyloxy)aniline (500 mg, 1.37 mmol, 1.0 eq.) and 2-(piperidin-1-yl)thiazole-4-carboxylic acid (436 mg, 2.06 mmol, 1.5 eq.) in THF (20 mL). The reaction was heated at 80° C. for 5 hours. The solution was poured into water and extracted with ethyl acetate for three times. The organic extracts were combined, washed with brine, dried over anhydrous sodium sulfate, and concentrated. The solid was stirred in CH₂Cl₂ (10 mL) for 10 min and filtered. The filtrate was concentrated, dissolved in CH₂Cl₂ (5 mL), and petroleum ether was added slowly. The residue was purified by silica gel column chromatography (petroleum ether/acetone=40:1 to 10:1) to give N-(3-(1,3-dithian-2-yl)-5-fluoro-4-(4-methoxybenzyloxy)phenyl)-2-(piperidin-1-yl)thiazole-4-carboxamide (610 mg, 1.09 mmol, 80% yield). LC-MS m/z [M+H]⁺ calc'd for C₂₇H₃₀FN₃O₃S₃, 560; found, 560.

Step 2: In a 100 mL glass vial, MeI (15.6 g, 0.11 mol, 100 eq.) was added to a mixture of N-(3-(1,3-dithian-2-yl)-5-fluoro-4-(4-methoxybenzyloxy)phenyl)-2-(piperidin-1-yl)thiazole-4-carboxamide (610 mg, 1.09 mmol, 1.0 eq.) and NaHCO₃ (1.83 g, 21.82 mmol, 20 eq.) in CH₃CN/water (30 mL/6 mL). The reaction was heated for 3 hours at 40° C. The reaction mixture was cooled to room temperature, poured into water, and extracted with ethyl acetate for three times. The organic extracts were combined, washed with brine, dried over anhydrous sodium sulfate, and concentrated. The residue was purified by silica gel column chromatography (petroleum ether/acetone/CH₂Cl₂=10:1:1 to 20:1:1) to give the intermediate (330 mg, 0.70 mmol, 65% yield) as a white solid. The intermediate (150 mg, 0.32 mmol) was dissolved in dioxane (2 mL) and 6 N HCl/dioxane (3 mL) was added. The reaction was stirred for 1 hour at room temperature. The resulting precipitate was collected by filtration and dried in vacuo to give N-(3-fluoro-5-formyl-4-hydroxyphenyl)-2-(piperidin-1-yl)thiazole-4-carboxamide hydrochloride (80 mg, 0.21 mmol, 66% yield) as an off-white solid. ¹H NMR (DMSO-d6, 400 MHz) δ: 10.31 (s, 1H), 9.99 (s, 1H), 8.06 (dd, J=13.2 Hz, 2.8 Hz, 1H), 7.97 (s, 1H), 7.56 (s, 1H), 3.53 (m, 4H), 1.62 (m, 6H). LC-MS m/z [M+H]⁺ calc'd for C₁₆H₁₆FN₃O₃S, 350; found, 350.

### Example 10: N-(3-fluoro-5-formyl-4-hydroxyphenyl)-5-phenylthiazole-2-carboxamide (Compound 10)

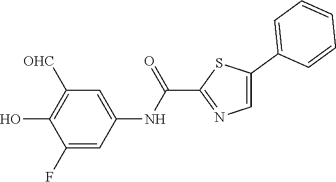 [SMILES: O=Cc1cc(NC(=O)c2ncc(-c3ccccc3)s2)cc(F)c1O]

The title compound was prepared from N-(3-(1,3-dithian-2-yl)-5-fluoro-4-(4-methoxybenzyloxy)phenyl)-5-phenylthiazole-2-carboxamide (480 mg, 0.87 mmol, 1.0 eq.) (prepared as in Example 9) using a method similar to that as described in Example 9 to give the final title compound hydrochloride salt (90 mg, 0.24 mmol, 58% yield) as a light yellow solid. ¹H NMR (DMSO-d6, 400 MHz) δ: 11.03 (s, 1H), 10.87 (br, 1H), 10.31 (s, 1H), 8.54 (s, 1H), 8.10 (m, 1H), 8.02 (dd, J=12.8 Hz, 2.8 Hz, 1H), 7.84 (m, 2H), 7.50 (m, 3H). LC-MS m/z [M+H]⁺ calc'd for C₁₇H₁₁FN₂O₃S, 343; found, 343.

### Example 11: 3-fluoro-N-(5-(4-fluorophenyl)thiazol-2-yl)-5-formyl-4-hydroxybenzamide (Compound 11)

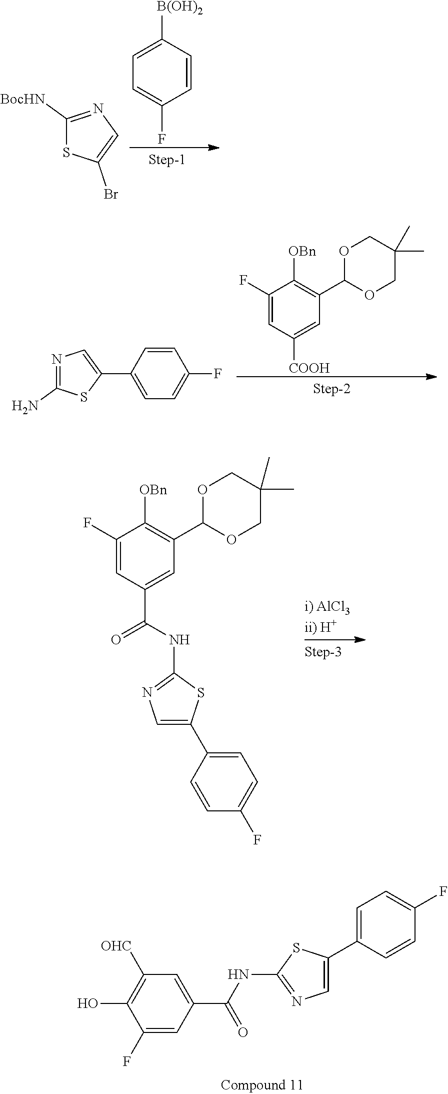

Step 1: Synthesis of 5-(4-fluorophenyl)thiazol-2-amine: A mixture of tert-butyl 5-bromothiazol-2-ylcarbamate (1 g, 3.6 mmol, 1.0 eq.), 4-fluorophenylboronic acid (550 mg, 3.9 mmol, 1.1 eq.), potassium carbonate (1.49 g, 10.8 mmol, 3 eq.), PdCl₂(dppf) (292 mg, 0.36 mmol, 0.1 eq.) in dioxane/water (15 mL/5 mL) was heated for 3 hours at 95° C. under N₂ protection. The mixture was cooled to room temperature and poured into water. The mixture was extracted with ethyl acetate for three times. The organic extracts were combined, washed with brine, dried over anhydrous sodium sulfate. The residue was purified by silica gel column chromatography (petroleum ether/EtOAc=10:1 to 2:1) to give tert-butyl 5-(4-fluorophenyl)thiazol-2-ylcarbamate (220 mg, 0.75 mmol, 21% yield). LC-MS m/z [M-tBu+H]⁺ calc'd for C₉H₇FN₂S, 239; found, 239. The resulted tert-Butyl 5-(4-fluorophenyl)thiazol-2-ylcarbamate (220 mg, 0.75 mmol, 1.0 eq.) was treated with 6 N HCl (gas) in dioxane for 30 min. The resulting mixture was concentrated, suspended in sat. sodium bicarbonate, and then extracted with ethyl acetate for three times. The organic extracts were combined, dried over anhydrous sodium sulfate, and concentrated to give 5-(4-fluorophenyl)thiazol-2-amine (140 mg, 0.72 mmol, 97% yield) used in next step.

Step 2: Synthesis of 4-(benzyloxy)-3-(5,5-dimethyl-1,3-dioxan-2-yl)-5-fluoro-N-(5-(4-fluorophenyl)thiazol-2-yl)benzamide: In a 50 mL glass vial, a solution of 4-(benzyloxy)-3-(5,5-dimethyl-1,3-dioxan-2-yl)-5-fluorobenzoic acid (260 mg, 0.72 mmol, 1.0 eq.) in dichloromethane (10 mL) was cooled to 0° C. Oxalyl chloride (183 mg, 1.44 mmol, 2.0 eq.) and 1 drop of DMF was added. The reaction was stirred for 2 hours at room temperature. The solvent was removed in vacuo and the residue was co-evaporated with dichloromethane for two times. The residue was re-dissolved in dichloromethane (2 mL) and added to a solution of 5-(4-fluorophenyl)thiazol-2-amine (140 mg, 0.72 mmol, 1.0 eq.) in THF (5 mL). Triethylamine (219 mg, 2.16 mmol, 3.0 eq.) was added and the reaction was stirred for 2 hours at room temperature. The solution was poured into water and extracted with ethyl acetate for three times. The organic extracts were combined, washed with brine, dried over anhydrous sodium sulfate, and concentrated. The residue was purified by silica gel column chromatography (petroleum ether/EtOAc=10:1 to 5:1) to give 4-(benzyloxy)-3-(5,5-dimethyl-1,3-dioxan-2-yl)-5-fluoro-N-(5-(4-fluorophenyl)thiazol-2-yl)benzamide (220 mg, 0.41 mmol, 57% yield). No LCMS was taken.

Step 3: 4-(Benzyloxy)-3-(5,5-dimethyl-1,3-dioxan-2-yl)-5-fluoro-N-(5-(4-fluorophenyl)thiazol-2-yl)benzamide (220 mg, 0.41 mmol, 1.0 eq.) was dissolved in dichloromethane (10 mL). Then AlCl₃ (110 mg, 0.82 mmol, 2.0 eq.) was added. The reaction was stirred for 2 hours at room temperature. The mixture was poured into water and extracted with ethyl acetate for three times. The organic extracts were combined, dried over anhydrous sodium sulfate, and concentrated. The residue was purified by silica gel column chromatography (petroleum ether/EtOAc=10:1 to 2:1) to give intermediate (72 mg, 0.16 mmol, 39% yield). The intermediate was dissolved in 4 N HCl aqueous solution/THF (10 mL/10 mL) and the reaction was stirred overnight at room temperature. Then pH of the system was adjusted to −7 and the mixture was extracted with ethyl acetate for three times. The organic extracts were combined, washed with brine, dried over anhydrous sodium sulfate, and concentrated. The residue was purified by silica gel column chromatography (petroleum ether/EtOAc=10:1 to 2:1) and slurried in PE/DCM (5 mL, 10:1) to give 3-fluoro-N-(5-(4-fluorophenyl)thiazol-2-yl)-5-formyl-4-hydroxybenzamide (11 mg, 0.03 mmol, 19% yield) as a yellow solid. ¹H NMR (DMSO-d6, 400 MHz) δ: 10.27 (br, 1H), 9.46 (br, 1H), 8.28 (s, 1H), 7.99 (m, 1H), 7.91 (s, 1H), 7.69 (m, 2H), 7.28 (m, 2H). LC-MS m/z [M+H]⁺ calc'd for C₁₇H₁₀F₂N₂O₃S, 361; found, 361.

### Example 12: 3-fluoro-5-formyl-4-hydroxy-N-(5-(3-(pyrrolidin-1-yl)phenyl)thiazol-2-yl)benzamide (Compound 12)

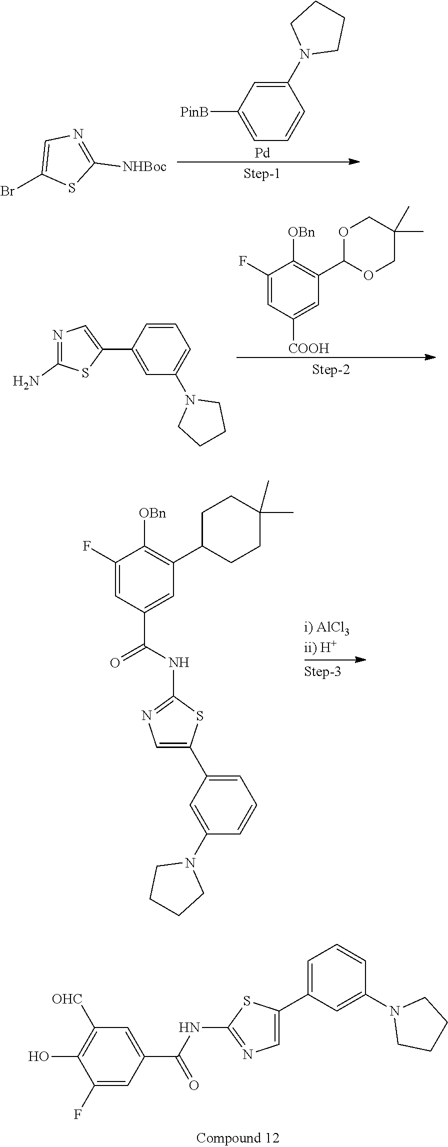

Step 1: Synthesis of 5-(3-(pyrrolidin-1-yl)phenyl)thiazol-2-amine: A mixture of tert-butyl 5-bromothiazol-2-ylcarbamate (1 g, 3.6 mmol, 1.0 eq.), 1-(3-(4,4,5,5-tetramethyl-1,3,2-dioxaborolan-2-yl)phenyl)pyrrolidine (1.07 g, 3.9 mmol, 1.1 eq.), potassium carbonate (1.49 g, 10.8 mmol, 3 eq.), PdCl₂(dppf) (584 mg, 0.72 mmol, 0.2 eq.) in dioxane/water (15 mL/5 mL) was heated overnight at 95° C. under N2 atmosphere. The mixture was cooled to room temperature and poured into water. The mixture was extracted with ethyl acetate for three times. The organic extracts were combined, washed with brine, dried over anhydrous sodium sulfate. The residue was purified by silica gel column chromatography (petroleum ether/EtOAc=10:1 to 2:1) to give tert-butyl 5-(3-(pyrrolidin-1-yl)phenyl)thiazol-2-ylcarbamate (280 mg, 0.8 mmol, 23% yield). LC-MS m/z [M-tBu+H]⁺ calc'd for C₁₈H₂₃N₃O₂S, 290; found, 290. Resulted tert-Butyl 5-(3-(pyrrolidin-1-yl)phenyl)thiazol-2-ylcarbamate (280 mg, 0.8 mmol, 1.0 eq.) was treated with 6 N HCl (gas) in dioxane for 30 min. The resulting mixture was concentrated, suspended in sat. sodium bicarbonate, and then extracted with ethyl acetate for three times. The organic extracts were combined, dried over anhydrous sodium sulfate, and concentrated to give 5-(3-(pyrrolidin-1-yl)phenyl)thiazol-2-amine (153 mg, 0.62 mmol, 77% yield).

Step 2: Synthesis of 4-(benzyloxy)-3-(5,5-dimethyl-1,3-dioxan-2-yl)-5-fluoro-N-(5-(3-(pyrrolidin-1-yl)phenyl)thiazol-2-yl)benzamide: In a 50 mL glass vial, a solution of 4-(benzyloxy)-3-(5,5-dimethyl-1,3-dioxan-2-yl)-5-fluorobenzoic acid (352 mg, 0.98 mmol, 3.0 eq.) in dichloromethane (10 mL) was cooled to 0° C. Oxalyl chloride (746 mg, 1.96 mmol, 6.0 eq.) and 1 drop of DMF was added. The reaction was stirred for 2 hours at room tempeature. The solvent was removed in vacuo and the residue was co-evaporated with dichloromethane for two times. The residue was re-dissolved in dichloromethane (2 mL) and added to a solution of 5-(3-(pyrrolidin-1-yl)phenyl)thiazol-2-amine (80 mg, 0.32 mmol, 1.0 eq.) in THF (5 mL). Triethylamine (165 mg, 1.63 mmol, 5 eq.) was added and the reaction was stirred for 2 hours at room temperature. The solution was poured into water, acidified to pH=3, and extracted with ethyl acetate for three times. The organic extracts were combined, washed with brine, dried over anhydrous sodium sulfate, and concentrated. The residue was slurried in petroleum ether/dichloromethane (5 mL, 10:1) to give 4-(benzyloxy)-3-(5,5-dimethyl-1,3-dioxan-2-yl)-5-fluoro-N-(5-(3-(pyrrolidin-1-yl)phenyl)thiazol-2-yl)benzamide (100 mg, 0.17 mmol, 53% yield). LC-MS m/z [M-tBu+H]⁺ calc'd for C₃₃H₃₄FN₃O₄S, 588; found, 588.

Step 3: 4-(Benzyloxy)-3-(5,5-dimethyl-1,3-dioxan-2-yl)-5-fluoro-N-(5-(3-(pyrrolidin-1-yl)phenyl)thiazol-2-yl)benzamide (90 mg, 0.15 mmol, 1.0 eq.) was dissolved in dichloromethane (10 mL). Then AlCl₃ (82 mg, 0.61 mmol, 4.0 eq.) was added. The reaction was stirred for 2 hours at room temperature. The mixture was poured into water and extracted with ethyl acetate for three times. The organic extracts were combined, dried over anhydrous sodium sulfate, and concentrated. The residue (73 mg) was dissolved in 4 N HCl aqueous solution/THF (10 mL/10 mL) and the reaction was stirred for 2 hours at room temperature. Then pH of the system was adjusted to −7 and the mixture was extracted with ethyl acetate for three times. The organic extracts were combined, washed with brine, dried over anhydrous sodium sulfate, and concentrated. The residue was slurried in petroleum ether/dichloromethane (5 mL, 10:1) to give 3-fluoro-5-formyl-4-hydroxy-N-(5-(3-(pyrrolidin-1-yl)phenyl)thiazol-2-yl)benzamide (20 mg, 0.05 mmol, 32% yield) as a yellow solid. ¹H NMR (DMSO-d6, 400 MHz) δ: 12.48 (br, 1H), 10.30 (s, 1H), 8.28 (s, 1H), 8.11 (d, J=12.4 Hz, 1H), 7.90 (s, 1H), 7.20 (d, J=8.0 Hz, 1H), 6.85 (d, J=7.2 Hz, 1H), 6.72 (s, 1H), 6.50 (d, J=8.0 Hz, 1H), 3.28 (m, 4H), 1.97 (m, 4H). LC-MS m/z [M+H]⁺ calc'd for C₂₁H₁₈FN₃O₃S, 412; found, 412.

### Example 13: 3-fluoro-5-formyl-4-hydroxy-N-(5-(4-(pyrrolidin-1-yl)phenyl)thiazol-2-yl)benzamide (Compound 13)

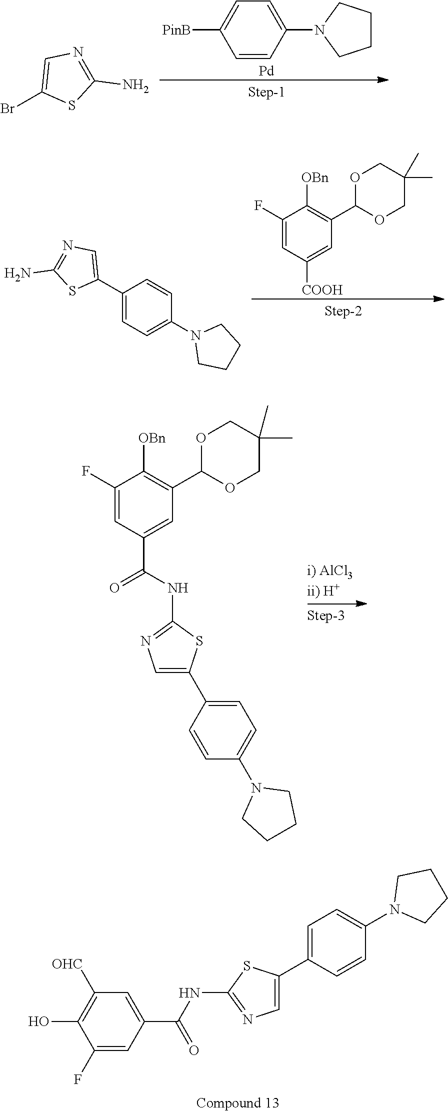

Step 1: Synthesis of 5-(4-(pyrrolidin-1-yl)phenyl)thiazol-2-amine: A mixture of 5-bromothiazol-2-amine (500 mg, 2.8 mmol, 1.0 eq.), 1-(4-(4,4,5,5-tetramethyl-1,3,2-dioxaborolan-2-yl)phenyl)pyrrolidine (837 mg, 3.1 mmol, 1.1 eq.), potassium carbonate (1.15 g, 8.3 mmol, 3.0 eq.), PdCl₂(dppf) (205 mg, 0.28 mmol, 0.1 eq.) in dioxane/water (12 mL/4 mL) was heated at 95° C. for 1.5 hours under N2 protection. The mixture was cooled to room temperature and poured into water. Then pH of the system was adjusted to 4-5 and extracted with ethyl acetate for three times. The organic extracts were combined, washed with brine, dried over anhydrous sodium sulfate. The residue was purified by silica gel column chromatography (petroleum ether/EtOAc=10:1 to 3:1) to give 5-(4-(pyrrolidin-1-yl)phenyl)thiazol-2-amine (310 mg, 1.3 mmol, 46% yield). LC-MS m/z [M+H]⁺ calc'd for C₁₃H₁₅N₃S, 246; found, 246.

Step 2: Synthesis of 4-(benzyloxy)-3-(5,5-dimethyl-1,3-dioxan-2-yl)-5-fluoro-N-(5-(4-(pyrrolidin-1-yl)phenyl)thiazol-2-yl)benzamide: The title compound was prepared from 4-(benzyloxy)-3-(5,5-dimethyl-1,3-dioxan-2-yl)-5-fluorobenzoic acid (220 mg, 0.6 mmol, 3.0 eq.) and 5-(4-(pyrrolidin-1-yl)phenyl)thiazol-2-amine (50 mg, 0.2 mmol, 1.0 eq.) using a method similar to that as described in Step 2 of Example 12 to give the 4-(benzyloxy)-3-(5,5-dimethyl-1,3-dioxan-2-yl)-5-fluoro-N-(5-(4-(pyrrolidin-1-yl)phenyl)thiazol-2-yl)benzamide (120 mg, 0.2 mmol, quantitative yield). LC-MS m/z [M+H]⁺ calc'd for C₃₃H₃₄FN₃O₄S, 588; found, 588.

Step 3: 4-(Benzyloxy)-3-(5,5-dimethyl-1,3-dioxan-2-yl)-5-fluoro-N-(5-(4-(pyrrolidin-1-yl)phenyl)thiazol-2-yl)benzamide (330 mg, 0.56 mmol, 1.0 eq.) was dissolved in dichloromethane (10 mL). Then AlCl₃ (742 mg, 5.62 mmol, 10 eq.) was added. The reaction was stirred for 2 hours at room temperature. The mixture was poured into water and extracted with ethyl acetate for three times. The organic extracts were combined, dried over anhydrous sodium sulfate, and concentrated. The residue (270 mg) was dissolved in 4 N HCl aqueous solution/THF (10 mL/10 mL) and the reaction was stirred overnight at room temperature. The pH of the system was adjusted to −7 and the mixture was extracted with ethyl acetate for three times. The organic extracts were combined, washed with brine, dried over anhydrous sodium sulfate, and concentrated. The residue was slurried in acetonitrile (3 mL) and petroleum ether/dichloromethane (5 mL, 10:1) to give 3-fluoro-5-formyl-4-hydroxy-N-(5-(4-(pyrrolidin-1-yl)phenyl)thiazol-2-yl)benzamide (85 mg, 0.21 mmol, 37% yield) as a red solid. ¹H NMR (DMSO-d6, 400 MHz) δ: 12.40 (br, 1H), 10.30 (s, 1H), 8.27 (s, 1H), 8.11 (d, J=12.4 Hz, 1H), 7.68 (s, 1H), 7.43 (d, J=8.4 Hz, 2H), 6.58 (d, J=8.4 Hz, 2H), 3.26 (m, 4H), 2.00 (m, 4H). LC-MS m/z [M+H]⁺ calc'd for C₂₁H₁₈FN₃O₃S, 412; found, 412.

### Example 14: 3-fluoro-2-hydroxy-5-(4-(pyrrolidin-1-yl)benzoyl)benzaldehyde (Compound 14)

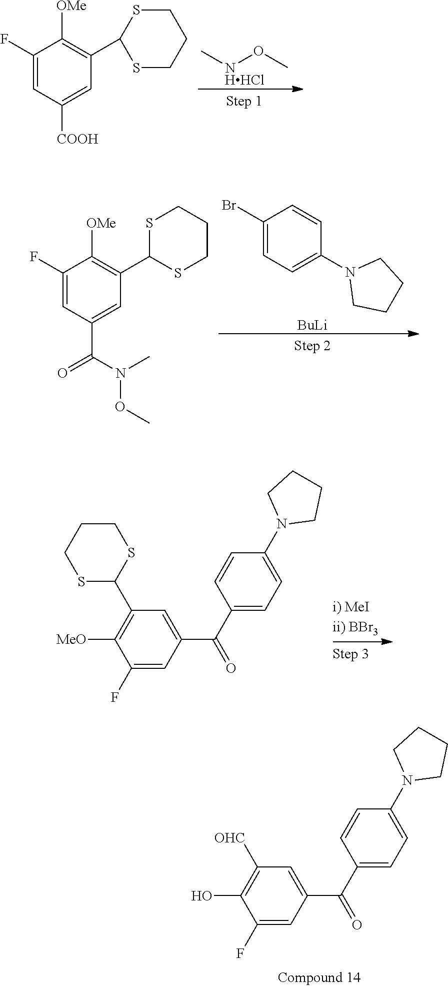

Step 1: Synthesis of 3-(1,3-dithian-2-yl)-5-fluoro-N,4-dimethoxy-N-methylbenzamide: In a 100 mL glass vial, CDI (1.88 g, 11.6 mmol, 1.2 eq.) was added to a solution of 3-(1,3-dithian-2-yl)-5-fluoro-4-methoxybenzoic acid (2.78 g, 9.65 mmol, 1.0 eq.) in CH₂Cl₂ (20 mL). The reaction was stirred for 2 hours at 0° C. Then N,O-dimethylhydroxylamine hydrochloride (1.4 g, 14.4 mmol, 1.5 eq.) was added. The reaction was stirred overnight at room temperature. The mixture was poured into water and extracted with CH₂Cl₂ for three times. The organic extracts were combined, washed with brine, dried over anhydrous sodium sulfate, and concentrated. The residue was purified by silica gel column chromatography (petroleum ether/EtOAc=25:1 to 12:1) to give 3-(1,3-dithian-2-yl)-5-fluoro-N,4-dimethoxy-N-methylbenzamide (1.57 g, 4.74 mmol, 49% yield). LC-MS m/z [M+H]⁺ calc'd for C₁₄H₁₈FNO₃S₂, 332; found, 332.

Step 2: Synthesis of (3-(1,3-dithian-2-yl)-5-fluoro-4-methoxyphenyl)(4-(pyrrolidin-1-yl)phenyl)methanone: In a 100 mL three-neck glass vial, BuLi (3.96 mL, 6.34 mmol, 1.6 M in hexane, 3.0 eq.) was added to a solution of 1-(4-bromophenyl)pyrrolidine (714 mg, 3.17 mmol, 1.5 eq.) in THF (20 mL) at −78° C. The reaction was stirred for 1.5 hours at −78° C. The a solution of 3-(1,3-dithian-2-yl)-5-fluoro-N,4-dimethoxy-N-methylbenzamide (700 mg, 2.11 mmol, 1.0 eq.) in THF (5 mL) was added. The reaction was stirred for 5 hours at room temperature. The mixture was poured into ice-cold sat. NH₄Cl and extracted with ethyl acetate for three times. The organic extracts were combined, washed with brine, dried over anhydrous sodium sulfate, and concentrated. The residue was purified by silica gel column chromatography (petroleum ether/EtOAc=40:1 to 30:1) to give (3-(1,3-dithian-2-yl)-5-fluoro-4-methoxyphenyl)(4-(pyrrolidin-1-yl)phenyl)methanone (380 mg, 0.91 mmol, 43% yield). LC-MS m/z [M+H]⁺ calc'd for C₂₂H₂₄FNO₂S2, 418; found, 418.

Step 3: In a 100 mL glass vial, MeI (11.9 g, 83.8 mol, 100.0 eq.) was added to a mixture of (3-(1,3-dithian-2-yl)-5-fluoro-4-methoxyphenyl)(4-(pyrrolidin-1-yl)phenyl)methanone (350 mg, 0.84 mmol, 1.0 eq.) and NaHCO₃ (1.41 g, 16.8 mmol, 20.0 eq.) in CH₃CN/water (30 mL/6 mL). The reaction was heated overnight at 40° C. The reaction mixture was cooled to room temperature, poured into water, and extracted with ethyl acetate for three times. The organic extracts were combined, washed with brine, dried over anhydrous sodium sulfate, and concentrated. The residue was purified by silica gel column chromatography (petroleum ether/EtOAc/CH₂Cl₂=35:1:1 to 10:1:1) to give the intermediate (205 mg, 0.63 mmol, 75% yield). The intermediate (150 mg, 0.46 mmol) was dissolved in was dissolved in CH₂Cl₂ (5 mL) and cooled to −78° C. Then BBr₃ (1.15 g, 4.6 mmol, 10 eq.) was added dropwise. The reaction was stirred overnight at rt. The reaction mixture was poured into ice-cold sat. sodium bicarbonate and extracted with CH₂Cl₂ for three times. The organic extracts were combined, washed with brine, dried over anhydrous sodium sulfate, and concentrated. The residue was purified by prep-TLC to give 3-fluoro-2-hydroxy-5-(4-(pyrrolidin-1-yl)benzoyl)benzaldehyde (100 mg, 0.32 mmol, 69% yield) as a green solid. ¹H NMR (DMSO-d6, 400 MHz) δ: 11.69 (br, 1H), 10.32 (s, 1H), 7.78 (m, 2H), 7.64 (d, J=8.8 Hz, 2H), 6.63 (d, J=8.8 Hz, 2H), 3.34 (m, 4H), 1.99 (m, 4H). LC-MS m/z [M+H]⁺ calc'd for C₁₈H₁₆FNO₃, 314; found, 314.

### Example 15: 3-fluoro-5-formyl-4-hydroxy-N-(2-phenyloxazol-4-yl)benzamide (Compound 15)

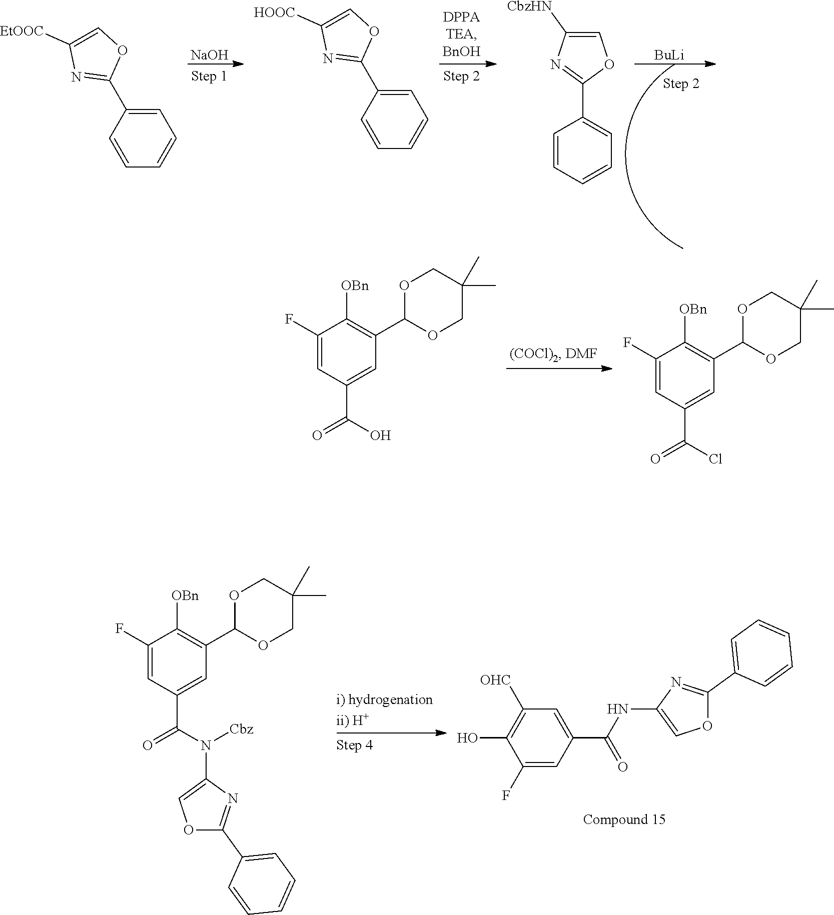

Step 1: Synthesis of 2-phenyloxazole-4-carboxylic acid: In a 100 mL glass vial, a solution of ethyl 2-phenyloxazole-4-carboxylate (2.17 g, 10.0 mmol, 1.0 eq.) in THF (10 mL) was treated with LiOH·H₂O (2.10 g, 50.0 mmol, 5.0 eq.) in water (10 mL). The reaction was heated for 3 hours at 80° C. Then pH of the system was adjusted to 4-5 with 5% KHSO₄ solution. The resulting mixture was then extracted with ethyl acetate for three times. The organic extracts were combined, washed with brine, dried over anhydrous sodium sulfate, and concentrated to give crude 2-phenyloxazole-4-carboxylic acid (1.93 g, quantitative yield), which was used for next reaction without further purification. LC-MS m/z [M−H]⁻ calc'd for C₁₀H₇NO₃, 188; found, 188.

Step 2: Synthesis of benzyl 2-phenyloxazol-4-ylcarbamate: In a 100 mL glass vial, a solution of 2-phenyloxazole-4-carboxylic acid (1.8 g, 9.5 mmol, 1.0 eq.), DPPA (3.41 g, 12.4 mmol, 1.3 eq.), and TEA (1.53 g, 15.2 mmol, 1.6 eq.) in BnOH (20 mL) was heated for 6 hours at 85° C. The solvent was removed in vacuo and the residue was dissolved in water/EtOAc. The mixture was then extracted with ethyl acetate for three times. The organic extracts were combined, washed with sat. sodium bicarbonate and brine, dried over anhydrous sodium sulfate, and concentrated. The residue was purified by silica gel column chromatography (petroleum ether/EtOAc=20:1 to 5:1) to give benzyl 2-phenyloxazol-4-ylcarbamate (2.31 g, 8.25 mmol, 75% yield). LC-MS m/z [M+H]⁺ calc'd for C₂₇H₂₁N₃O₆, 295; found, 295.

Step 3: Synthesis of benzyl (4-(benzyloxy)-3-(5,5-dimethyl-1,3-dioxan-2-yl)-5-fluorobenzoyl)(2-phenyloxazol-4-yl)carbamate: In a 50 mL glass vial, oxalyl chloride (441 mg, 3.47 mmol, 2.0 eq.) was added to a solution of 4-(benzyloxy)-3-(5,5-dimethyl-1,3-dioxan-2-yl)-5-fluorobenzoic acid (625 mg, 1.74 mmol, 1.0 eq.) in dichloromethane (15 mL). A drop of DMF was added and the reaction was stirred for 2 hours at room temperature. The solvent was removed in vacuo and the residue was co-evaporated with dichloromethane for two times. In a 100 mL three-neck glass bottle, benzyl 2-phenyloxazol-4-ylcarbamate (340 mg, 1.16 mmol, 0.67 eq.) was dissolved in THF (10 mL) under nitrogen atmosphere. The solution was cooled to −78° C. and BuLi (1.45 mL, 1.6 M in THF/hexane, 2.32 mmol, 1.33 eq.) was added. The reaction was stirred for 1.5 hours at −40° C. Then, a solution of the intermediate in vial 1 in THF (5 mL) was added dropwise. The reaction was then stirred overnight at room temperature. The mixture was poured into sat. NH₄Cl and extracted with ethyl acetate for three times. The organic extracts were combined, washed with brine, dried over anhydrous sodium sulfate, and concentrated. The residue was purified by silica gel column chromatography (PE/EA=100:1 to 20:1) to give benzyl 4-(benzyloxy)-3-(5,5-dimethyl-1,3-dioxan-2-yl)-5-fluorobenzoyl(2-phenyloxazol-4-yl)carbamate (210 mg, 29% yield) as a yellow oil. LC-MS m/z [M+H]⁺ calc'd for C₃₇H₃₃FN₂O₇, 637; found, 637.

Step 4: In a 100 mL glass vial, a mixture of benzyl 4-(benzyloxy)-3-(5,5-dimethyl-1,3-dioxan-2-yl)-5-fluorobenzoyl(2-phenyloxazol-4-yl)carbamate (210 mg, 0.33 mmol, 1.0 eq.) and Pd/C (100 mg) in THF (10 mL) was hydrogenated for 2 hours at room temperature. Then, Pd/C was filtered off and the filtrate was concentrated in vacuo to give intermediate (200 mg, quantitative yield). The intermediate (crude 200 mg, 0.33 mmol) was dissolved in THF (5 mL) and 4 N HCl (5 mL) was added. The reaction was stirred overnight at room temperature. The mixture was neutralized with sodium bicarbonate, extracted with ethyl acetate for three times. The organic extracts were combined, washed with brine, dried over anhydrous sodium sulfate, and concentrated. The residue was purified by prep-TLC and slurried in petroleum ether/dichloromethane to give 3-fluoro-5-formyl-4-hydroxy-N-(2-phenyloxazol-4-yl)benzamide (2 mg, 0.006 mmol, 2% yield) as a yellow solid. ¹H NMR (DMSO-d6, 400 MHz) δ: 11.42 (br, 1H), 10.32 (br, 1H), 8.38 (s, 1H), 8.28 (s, 1H), 8.19 (d, J=11.2 Hz, 1H), 7.99 (dd, J=7.6 Hz, 2.8 Hz, 2H), 7.57 (m, 3H). LC-MS m/z [M+H]⁺ calc'd for C₁₇H₁₁FN₂O₄, 327; found, 327.

### Example 16: 3-fluoro-2-hydroxy-5-(4-phenylthiazol-5-yl)benzaldehyde (Compound 18)

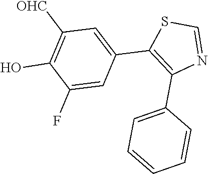 [SMILES: O=Cc1cc(-c2scnc2-c2ccccc2)cc(F)c1O]

The title compound was prepared using a method similar to that as described for Example 16, using commercially available 5-bromo-4-phenylthiazole. Yield: 52%. ¹H NMR (500 MHz, DMSO-d6) δ 11.29 (s, 1H), 10.25 (s, 1H), 9.19 (s, 1H), 7.52-7.40 (m, 4H), 7.41-7.29 (m, 3H). LC-MS m/z [M+H]‘ calc’d for C₁₆H₁₀FNO₂S, 300; found, 300.

### Example 17: 3-fluoro-5-formyl-4-hydroxy-N-(6-(pyrrolidin-1-yl)-5-(trifluoromethyl)pyridin-3-yl)benzamide (Compound 19)

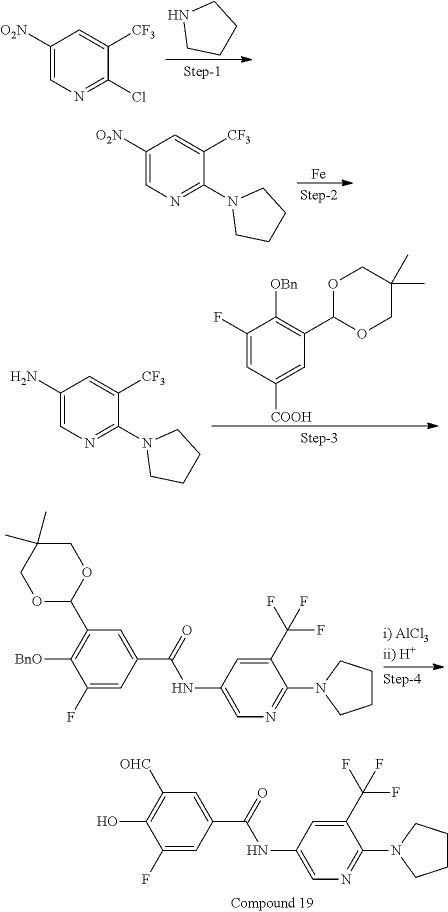

Step 1: Synthesis of 5-nitro-2-(pyrrolidin-1-yl)-3-(trifluoromethyl)pyridine: In a 100 mL glass vial, a mixture of 2-chloro-5-nitro-3-(trifluoromethyl)pyridine (1.0 g, 4.4 mmol, 1.0 eq.) pyrrolidine (317 mg, 4.4 mmol, 1.0 eq.), and K₂CO₃ (1.83 g, 13.2 mmol, 3.0 eq.) in DMF (10 mL) was heated for 4 hours at 85° C. The reaction mixture was cooled to room temperature, poured into water, and extracted with ethyl acetate for three times. The organic extracts were combined, washed with brine, dried over anhydrous sodium sulfate, and concentrated to give crude 5-nitro-2-(pyrrolidin-1-yl)-3-(trifluoromethyl)pyridine (1.1 g, 4.2 mmol, 96% yield), which was used for next reaction without further purification. No LCMS was taken for this compound.

Step 2: Synthesis of 6-(pyrrolidin-1-yl)-5-(trifluoromethyl)pyridin-3-amine: In a 100 mL glass vial, iron powder (2.36 g, 42.1 mmol, 10 eq.) was added to a solution of 5-nitro-2-(pyrrolidin-1-yl)-3-(trifluoromethyl)pyridine (1.1 g, 4.2 mmol, 1.0 eq.) in AcOH (30 mL). The reaction was heated for 30 min at 60° C. The reaction mixture was cooled to room temperature, poured into sat. NaHCO₃ and extracted with ethyl acetate for three times. The organic extracts were combined, washed with brine, dried over anhydrous sodium sulfate, and concentrated to give crude 6-(pyrrolidin-1-yl)-5-(trifluoromethyl)pyridin-3-amine (810 mg, 3.5 mmol, 83% yield), which was used for next reaction without further purification. LC-MS m/z [M+H]⁺ calc'd for C₁₀H₁₂F₃N₃, 232; found, 232.

Step 3: Synthesis of 4-(benzyloxy)-3-(5,5-dimethyl-1,3-dioxan-2-yl)-5-fluoro-N-(6-(pyrrolidin-1-yl)-5-(trifluoromethyl)pyridin-3-yl)benzamide: In a 50 mL glass vial, a solution of 4-(benzyloxy)-3-(5,5-dimethyl-1,3-dioxan-2-yl)-5-fluorobenzoic acid (297 mg, 0.83 mmol, 1.0 eq.) in dichloromethane (10 mL) was cooled to 0° C. Oxalyl chloride (210 mg, 1.66 mmol, 2.0 eq.) and 1 drop of DMF was added. The reaction was stirred for 2 hours at room temperature. The solvent was removed in vacuo and the residue was co-evaporated with dichloromethane for two times. The residue was re-dissolved in dichloromethane (2 mL) and added to a solution of 6-(pyrrolidin-1-yl)-5-(trifluoromethyl)pyridin-3-amine (200 mg, 0.83 mmol, 1.0 eq.) in THF (5 mL) and the solution was cooled to 0° C. Triethylamine (250 mg, 2.5 mmol, 3 eq.) was added and the reaction was stirred for 30 min at room temperature. The solution was poured into water and extracted with ethyl acetate for three times. The organic extracts were combined, washed with brine, dried over anhydrous sodium sulfate, and concentrated. The residue was purified by silica gel column chromatography (petroleum ether/EtOAc=10:1 to 2:1) to give 4-(benzyloxy)-3-(5,5-dimethyl-1,3-dioxan-2-yl)-5-fluoro-N-(6-(pyrrolidin-1-yl)-5-(trifluoromethyl)pyridin-3-yl)benzamide (370 mg, 0.65 mmol, 78% yield).

Step 4: 4-(Benzyloxy)-3-(5,5-dimethyl-1,3-dioxan-2-yl)-5-fluoro-N-(6-(pyrrolidin-1-yl)-5-(trifluoromethyl)pyridin-3-yl)benzamide (360 mg, 0.63 mmol, 1.0 eq.) was dissolved in dichloromethane (10 mL). Then, AlCl₃ (334 mg, 2.51 mmol, 4 eq.) was added. The reaction was stirred for 2 hours at room temperature. The mixture was poured into water and extracted with ethyl acetate for three times. The organic extracts were combined, dried over anhydrous sodium sulfate, and concentrated. The residue was purified by silica gel column chromatography (petroleum ether/EtOAc=10:1 to 2:1) to give intermediate (130 mg, 0.27 mmol, 42% yield). The intermediate (130 mg, 0.27 mmol) was dissolved in 4 N HCl aqueous solution/THF (10 mL/10 mL) and the reaction was stirred overnight at room temperature. Then, pH of the system was adjusted to −7 and the mixture was extracted with ethyl acetate for three times. The organic extracts were combined, washed with brine, dried over anhydrous sodium sulfate, and concentrated. The residue was purified by silica gel column chromatography (petroleum ether/EtOAc=10:1 to 1:1) to give 3-fluoro-5-formyl-4-hydroxy-N-(6-(pyrrolidin-1-yl)-5-(trifluoromethyl)pyridin-3-yl)benzamide (30 mg, 0.08 mmol, 28% yield) as a yellow solid. ¹H NMR (DMSO-d6, 400 MHz) δ: 11.74 (br, 1H), 10.39 (s, 1H), 10.36 (s, 1H), 8.67 (s, 1H), 8.21 (s, 1H), 8.08 (dd, J=12.0 Hz, 2.0 Hz, 1H), 3.54 (m, 4H), 1.89 (m, 4H). LC-MS m/z [M+H]⁺ calc'd for C₁₈H₁₅F₄N₃O₃, 398; found, 398.

### Example 18: 3-fluoro-2-hydroxy-5-(4-(4-(pyrrolidin-1-yl)phenyl)piperidine-1-carbonyl)benzaldehyde (Compound 34)

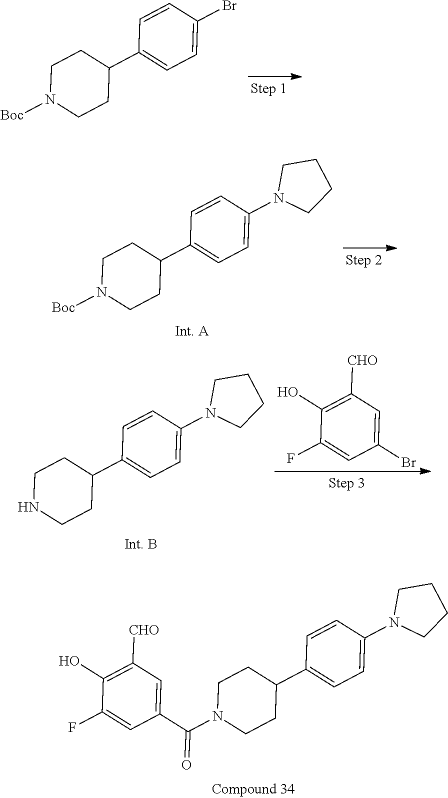

Step 1: Synthesis of tert-butyl 4-(4-(pyrrolidin-1-yl)phenyl)piperidine-1-carboxylate: The procedure used to make tert-butyl 4-(4-(pyrrolidin-1-yl)phenyl)piperidine-1-carboxylate was similar to that used by Watanabe et al. (PTC Int. Appl. 2004085405 07 October, 2004.) A suspension of 4-(4-Bromophenyl)-piperidine-1-carboxylic acid tert-butyl ester (255 mg, 0.75 mmol), palladium acetate (8.42 mg), Xphos (35.8 mg), and potassium t-butoxide (126 mg), pyrrolidine (86 mL, 1.05 mmol) in toluene (5 mL) was heated at 90° C. for 4 hours under nitrogen atmosphere. The resulting suspension was passed through a celite column and partitioned between ethyl acetate and water. The aqueous layer was extracted with ethyl acetate. The combined organic layer was washed with brine, dried over sodium sulfate, and concentrated in vacuo. Purification of the residue flash column chromatography (0-3% MeOH/DCM) furnished 4-(4-Pyrrolidin-1-yl-phenyl)-piperidine-1-carboxylic acid tert-butyl ester (100 mg, 40% yield). ¹H NMR (499 MHz, Chloroform-d) δ 7.15-6.91 (m, 2H), 6.54 (s, 2H), 4.22 (s, 2H), 3.26 (d, J=6.2 Hz, 4H), 2.78 (s, 2H), 2.56-2.40 (m, 1H), 2.09-1.90 (m, 4H), 1.79 (d, J=13.4 Hz, 2H), 1.68-1.49 (m, 2H), 1.48 (s, 9H).

Step 2: Synthesis of 4-(4-(pyrrolidin-1-yl)phenyl)piperidine: 4-(4-(pyrrolidin-1-yl)phenyl)piperidine-1-carboxylate (100 mg, 0.3 mmol) was dissolved in 0.3 mL dioxane and cooled to 0° C. before adding 0.6 mL of 4N HCl in Dioxane. The reaction mixture was allowed to come to room temperature and stirred 1 hour until no starting material was visible by TLC. The solvent was evaporated and the residue was taken up in water and washed with ethyl acetate. The organic layer was discarded and the aqueous layer was basified to pH=10 with 1M NaOH. The aqueous layer was extracted 3 times with EtOAc. The combined organic extracts were dried over Na₂SO₄, filtered and concentrated. The 4-(4-(pyrrolidin-1-yl)phenyl)piperidine was used as is in the next reaction.

Step 3: The aminocrbonylation was accomplished in the same manner described by Nordeman et al. (J. Org. Chem. 2012, 77, 11393-11398.) using sealed 2 chamber COware gas reactor (Sigma Aldrich). To chamber one (C-1) was added Mo(CO)₆ (47 mg, 0.18 mmol). To chamber two (C-2) was added with 5-bromo-3fluoro-salicaldehyde (39 mg, 0.18 mmol), Pd(dppf)Cl₂ (8.3 mg), 4-(4-(pyrrolidin-1-yl)phenyl)piperidine (41 mg, 0.18 mmol), and DMAP (23 mg). Dioxane (0.6 mL each) was added to both C-1 and C-2. Triethylamine (74 μL) was added to C-2 before both chamber sides were capped. The reactor was flushed with nitrogen. DBU (80 μL, 0.54 mmol) was added to C-1. The reactor was heated in a heating block at 90° C. overnight with vigorous stirring. After carefully degassing the chambers, the crude mixture from C₂ was diluted with DCM, transferred to a separatory funnel, acidified to pH 3 with 10% citric acid and extracted three times with DCM. The combined organic extracts were dried over Na₂SO₄, filtered and evaporated to dryness. The residue was purified by column chromatography (0-10% MeOH/DCM). Some impurities remained, so the product was taken up in DCM and hexane was added until a precipitate began to form. The product remained in the supernatant. The mixture was centrifuged, the supernatant was transferred to a round bottom flask and the solvent removed in vacuo to provide 3-fluoro-2-hydroxy-5-(4-(4-(pyrrolidin-1-yl)phenyl)piperidine-1-carbonyl)benzaldehyde (16.5 mg, 23% yield). ¹H NMR (500 MHz, DMSO-d6) δ 11.32 (s, 1H), 10.29 (s, 1H), 7.63 (dd, J=11.1, 2.1 Hz, 1H), 7.57-7.45 (m, 1H), 7.17-7.00 (m, 2H), 6.52-6.38 (m, 2H), 3.33 (bs, 2H), 3.20-3.09 (m, 4H), 3.00 (bs, 2H), 2.76-2.61 (m, 1H), 1.95-1.84 (m, 4H), 1.73 (s, 2H), 1.59-1.42 (m, 2H). LC-MS m/z [M+H]⁺ calc'd for C₂₃H₂₅FN₂O₃, 397; Found, 397.

### Example 19: 3-fluoro-2-hydroxy-5-(4-(3-(pyrrolidin-1-yl)phenyl)piperidine-1-carbonyl)benzaldehyde (Compound 35

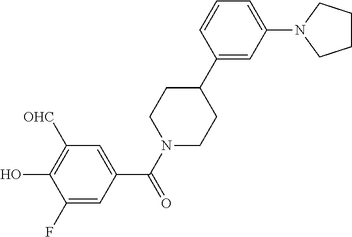 [SMILES: O=Cc1cc(C(=O)N2CCC(c3cccc(N4CCCC4)c3)CC2)cc(F)c1O]

3-fluoro-2-hydroxy-5-(4-(3-(pyrrolidin-1-yl)phenyl)piperidine-1-carbonyl)benzaldehyde was prepared using a method similar to that as described in Example 20 from tert-butyl 4-(3-(pyrrolidin-1-yl)phenyl)piperidine-1-carboxylate and 5-bromo-3fluoro-salicaldehyde to provide 32 mg of the desired product in an 9.5% yield over 3 steps. ¹H NMR (500 MHz, Chloroform-d) δ 11.13 (s, 1H), 9.95 (d, J=1.7 Hz, 1H), 7.57 (dd, J=2.1, 1.0 Hz, 1H), 7.48 (dd, J=10.6, 2.0 Hz, 1H), 7.18 (t, J=7.8 Hz, 1H), 6.52 (d, J=7.6 Hz, 1H), 6.45 (dd, J=8.2, 2.4 Hz, 1H), 6.39 (t, J=2.0 Hz, 1H), 3.32-3.05 (m, 8H), 2.80-2.69 (m, 1H), 2.05-1.86 (m, 8H). LC-MS m/z [M+H]⁺ calc'd for C₂₃H₂₅FN₂O₃, 397; found, 397.

### Example 20: 3-fluoro-2-hydroxy-5-(4-(3-(pyrrolidin-1-yl)phenyl)piperidine-1-carbonyl)benzaldehyde (Compound 47)

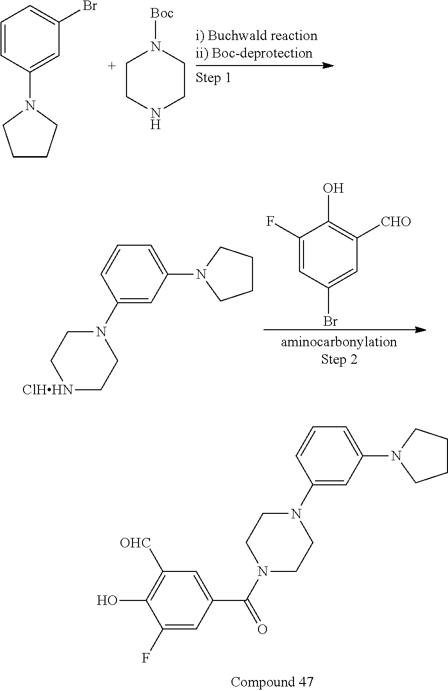

Step 1: Synthesis of 1-(3-(pyrrolidin-1-yl)phenyl)piperazine hydrochloride: In a 100 mL sealed cap glass vial, 1-(3-bromophenyl)pyrrolidine (904 mg, 4.0 mmol, 1.0 eq.), tert-butyl piperazine-1-carboxylate (744 mg, 4.0 mmol, 1.0 eq.), palladium acetate (88 mg, 0.4 mmol, 0.1 eq.), BINAP (496 mg, 0.8 mmol, 0.2 eq.) and t-BuOK (672 mg, 6.0 mmol, 1.5 eq.) were suspended in toluene (25 mL). Then, heated white stirring for 16 hours at 105° C. Cooled to room temperature and added water (50 mL) and extracted with EtOAc (2×60 mL) using separating funnel. Combined organic layer washed with brine, dried over Na₂SO₄ and evaporated. The resulting crude mixture purified by silica gel column chromatography using petroleum ether-EtOAc (0-80%). Pure product fractions combined and evaporated to give tert-butyl 4-(3-(pyrrolidin-1-yl)phenyl)piperazine-1-carboxylate as off-white solid (1.05 g, 3.17 mmol, 79% yield). The resulting product was dissolved in dichloromethane (15 mL) treated with 4N HCl in dioxane (15 mL) at room temperature to yield the desired hydrochloride intermediate in quantitative yield.

Step 2: The title compound was prepared from 5-bromo-3-fluoro-2-hydroxybenzaldehyde (110 mg, 0.5 mmol, 1.0 eq.) and 1-(3-(pyrrolidin-1-yl)phenyl)piperazine hydrochloride (134 mg, 0.5 mmol, 1.0 eq.) using a method similar to that as described in Step 3 of Example 20. Desired product obtained as light yellow solid (21 mg, 11% yield). ¹H NMR (500 MHz, Chloroform-d) δ 11.16 (s, 1H), 9.97 (s, 1H), 7.64-7.54 (m, 1H), 7.48 (dd, J=10.4, 2.0 Hz, 1H), 7.14 (t, J=8.1 Hz, 1H), 6.40-5.96 (m, 3H), 4.28-3.37 (m, 4H), 3.36-3.03 (m, 8H), 2.12-1.85 (m, 4H). LC-MS m/z [M+H]⁺ calc'd for C₂₂H₂₄FN₃O₃, 398; found, 398.

### Example 21: 3-fluoro-2-hydroxy-5-(3-(4-(pyrrolidin-1-yl)phenyl)pyrrolidine-1-carbonyl)benzaldehyde (Compound 82)

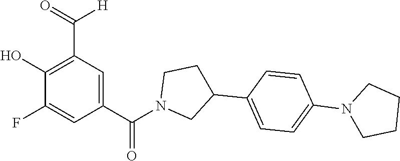 [SMILES: O=Cc1cc(C(=O)N2CCC(c3ccc(N4CCCC4)cc3)C2)cc(F)c1O]

The title compound was prepared in a similar manner to Example 20 starting from tert-Butyl 3 tert-Butyl 3-(4-bromophenyl)pyrrolidine-1-carboxylate and 5-bromo-3-fluoro-2-hydroxybenzaldehyde to provide the 40 mg of the desired compound in 32% yield over 3 steps. ¹H NMR (499 MHz, Chloroform-d) δ 11.15 (s, 1H), 10.05-9.84 (m, 1H), 7.69 (d, J=16.9 Hz, 1H), 7.66-7.56 (m, 1H), 7.16-7.02 (m, 2H), 6.54 (dd, J=15.3, 8.0 Hz, 2H), 4.10-3.30 (m, 4H), 3.30-3.23 (m, 5H), 2.41-2.19 (m, 1H), 2.18-1.91 (m, 5H). MS m/z [M+H]⁺ calc'd for C₂₂H₂₃FN₂O₃, 383; found, 383.

### Example 22: 3-fluoro-2-hydroxy-5-(3-(4-(pyrrolidin-1-yl)phenyl)azetidine-1-carbonyl)benzaldehyde (Compound 84)

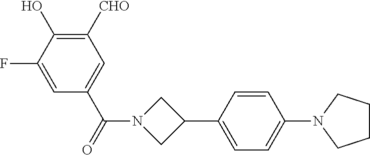 [SMILES: O=Cc1cc(C(=O)N2CC(c3ccc(N4CCCC4)cc3)C2)cc(F)c1O]

The title compound was prepared in a similar manner to Example 20 starting from tert-butyl 3-(4-(pyrrolidin-1-yl)phenyl)azetidine-1-carboxylate and 5-bromo-3-fluoro-2-hydroxybenzaldehyde to provide the desired 21 mg of compound in 12% overall yield over 3 steps. ¹H NMR (500 MHz, Chloroform-d) δ 11.22 (s, 1H), 10.06-9.72 (m, 1H), 7.88-7.77 (m, 1H), 7.71 (dd, J=10.9, 2.0 Hz, 1H), 7.23-7.13 (m, 2H), 6.72-6.25 (m, 2H), 4.65 (d, J=57.5 Hz, 2H), 4.30 (d, J=32.1 Hz, 2H), 3.84 (tt, J=9.0, 6.3 Hz, 1H), 3.38-3.18 (m, 4H), 2.09-1.94 (m, 4H). MS m/z [M+H]⁺ calc'd for C₂₁H₂₁FN₂O₃, 369; found, 369.

### Example 23: 3-fluoro-5-formyl-4-hydroxy-N-(6-(pyrrolidin-1-yl)pyridazin-3-yl)benzamide (Compound 92)

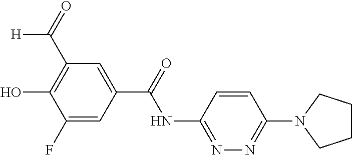 [SMILES: O=Cc1cc(C(=O)Nc2ccc(N3CCCC3)nn2)cc(F)c1O]

3-fluoro-5-formyl-4-hydroxy-N-(6-(pyrrolidin-1-yl)pyridazin-3-yl)benzamide was synthesized as described in Example 20. The equivalent of Int. B, 6-(pyrrolidin-1-yl)pyridazin-3-amine, was synthesized in a similar manner described by Finlay et al. (Journal of Medicinal Chemistry, 2019, 62(14), 6540-6560). 6-Chloropyridazin-3-amine (320 mg, 2.5 mmol), pyrrolidine (615 μL, 7.5 mmol), triethylamine (0.7 mL, 5 mmol) and n-BuOH (2 mL) were combined and heated in a microwave reactor to 165° C. for 100 minutes. The residue was concentrated in vacuo and purified by column chromatography (10% MeOH/DCM with 0.1% NH4OH), to give the intermediate, 6-(pyrrolidin-1-yl)pyridazin-3-amine, in 73% yield (302 mg). Aminocarbonylation of the resultant 6-(pyrrolidin-1-yl)pyridazin-3-amine (150 mg, 0.9 mmol) with 5-bromo-3fluoro-salicaldehyde (200 mg, 0.9 mmol) gave 3-fluoro-5-formyl-4-hydroxy-N-(6-(pyrrolidin-1-yl)pyridazin-3-yl)benzamide (20 mg, 6.5% yield). For this reaction, precipitation in DCM and Hexane resulted in product in the precipitate, not the supernatant. ¹H NMR (500 MHz, DMSO-d6) δ 10.99 (s, 1H), 10.32 (s, 1H), 8.25 (d, J=2.2 Hz, 1H), 8.17 (dd, J=11.8, 2.3 Hz, 1H), 7.94 (d, J=9.6 Hz, 1H), 6.99 (d, J=9.7 Hz, 1H), 3.51-3.43 (m, 4H), 2.03-1.93 (m, 4H). LC-MS m/z [M+H]⁺ calc'd for C₁₆H₁₅FN₄O₃, 331; found, 331.

### Example 24: 3-fluoro-5-formyl-4-hydroxy-N-(5-(4-(trifluoromethyl)phenyl)thiazol-2-yl)benzamide (Compound 115)

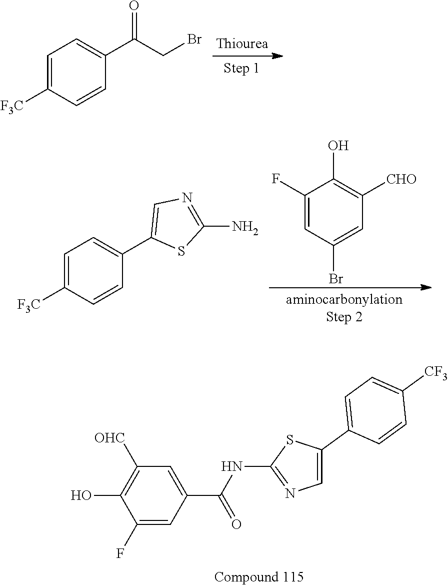 [SMILES: O=C(CBr)c1ccc(C(F)(F)F)cc1 ; Nc1ncc(-c2ccc(C(F)(F)F)cc2)s1 ; O=Cc1cc(Br)cc(F)c1O ; O=Cc1cc(C(=O)Nc2ncc(-c3ccc(C(F)(F)F)cc3)s2)cc(F)c1O]

Step 1: Synthesis of 5-(4-(trifluoromethyl)phenyl)thiazol-2-amine: In a 50 mL round bottom flask, 2-bromo-1-(4-(trifluoromethyl)phenyl)ethan-1-one (801 mg, 3.0 mmol, 1.0 eq.) and thiourea (297 mg, 3.9 mmol, 1.3 eq.) were suspended in EtOH (15 mL). Then refluxed at 90° C. overnight. Then cooled the reaction mixture to room temperature and added to aqueous sat. NaHCO₃ solution. Extracted the aqueous layer twice with ethyl acetate (2×50 mL). Combined organic layer washed with brine and dried over Na₂SO₄ and evaporated to get the desired product 5-(4-(trifluoromethyl)phenyl)thiazol-2-amine (732 mg, quantitative yield) as off-white solid. LC-MS m/z [M+H]⁺ calc'd for C₁₀H₇F₃N₂S, 245; found, 245.

Step 2: The title compound was prepared from 5-bromo-3-fluoro-2-hydroxybenzaldehyde (110 mg, 0.5 mmol, 1.0 eq.) and 5-(4-(trifluoromethyl)phenyl)thiazol-2-amine (123 mg, 0.5 mmol, 1.0 eq.) using a method similar to that as described in Step 3 of Example 20. Desired product obtained as yellow solid (25 mg, 12% yield). ¹H NMR (500 MHz, Chloroform-d) δ 11.19 (br s, 2H), 9.92 (s, 1H), 7.99 (s, 1H), 7.84 (m, 3H), 7.61 (d, J=7.7 Hz, 2H), 7.34 (s, 1H). LC-MS m/z [M+H]⁺ calc'd for C₁₈H₁₀F₄N₂O₃S, 411; found, 411.

### Example 25: 3-fluoro-2-hydroxy-5-(1-(4-(trifluoromethyl)phenyl)-1H-pyrazol-4-yl)benzaldehyde (Compound 127)

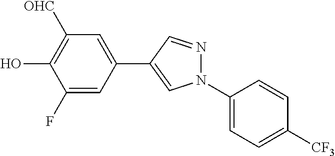 [SMILES: O=Cc1cc(-c2cnn(-c3ccc(C(F)(F)F)cc3)c2)cc(F)c1O]

In a 30 mL sealed cap glass vial, 5-bromo-3-fluoro-2-hydroxybenzaldehyde (219 mg, 1.0 mmol, 1.0 eq.), 4-(4,4,5,5-tetramethyl-1,3,2-dioxaborolan-2-yl)-1-(4-(trifluoromethyl)phenyl)-1H-pyrazole (372 mg, 1.1 mmol, 1.1 eq.) and Na₂CO₃ (666, 6.0 mmol, 6.0 eq.) were combined in dioxane-water (1:1) (20 mL). Then passed Argon gas for 2 min into the mixture, then added Pd(PPh₃)₄ (63 mg, 0.05 mmol, 0.05 eq) to the reaction mixture. Closed the reaction vial with sealed cap and heated at 105° C. for 16 hours. Then cooled to room temperature, added water (20 mL) to the reaction mixture, acidified with 10% citric acid and extracted into EtOAc twice (2×60 mL). Combined organic layer washed with brine, dried over sodium sulfate and evaporated to give a crude mixture. Then purified using silica gel column chromatography with hexane-EtOAc (0-80%) as a solvent system. Pure product fractions combined and evaporated to give the desired title product (135 mg, 0.38 mmol, 38% yield) as off-white solid. ¹H NMR (500 MHz, Chloroform-d) δ 10.92 (s, 1H), 10.00 (d, J=1.9 Hz, 1H), 8.19 (s, 1H), 7.99 (s, 1H), 7.87 (d, J=8.6 Hz, 2H), 7.76 (d, J=8.5 Hz, 2H), 7.59-7.49 (m, 2H). LC-MS m/z [M+H]⁺ calc'd for C₁₇H₁₀F₄N₂O₂, 351; found, 351.

### Example 26: 2-hydroxy-3-methoxy-5-(1-(4-(trifluoromethyl)phenyl)-1H-pyrazol-4-yl)benzaldehyde (Compound 128)

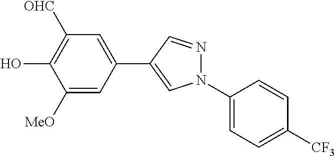 [SMILES: COc1cc(-c2cnn(-c3ccc(C(F)(F)F)cc3)c2)cc(C=O)c1O]

The title compound was prepared from 5-bromo-3-fluoro-2-hydroxybenzaldehyde (219 mg, 1.0 mmol, 1.0 eq.) and 4-(4,4,5,5-tetramethyl-1,3,2-dioxaborolan-2-yl)-1-(4-(trifluoromethyl)phenyl)-1H-pyrazole (372 mg, 1.1 mmol, 1.1 eq.) using a method similar to that as described in Example 27 to give 2-hydroxy-3-methoxy-5-(1-(4-(trifluoromethyl)phenyl)-1H-pyrazol-4-yl)benzaldehyde (165 mg, 0.46 mmol, 46% yield). ¹H NMR (500 MHz, Chloroform-d) δ 11.02 (s, 1H), 9.99 (s, 1H), 8.19 (s, 1H), 8.00 (s, 1H), 7.88 (d, J=8.5 Hz, 2H), 7.75 (d, J=8.5 Hz, 2H), 7.36 (d, J=1.9 Hz, 1H), 4.01 (s, 3H). LC-MS m/z [M+H]⁺ calc'd for C₁₈H₁₃F₃N₂O₃, 363; found, 363.

### Example 27: 5-hydroxy-2-(2-(6-(pyrrolidin-1-yl)pyridin-3-yl)thiazol-5-yl)isonicotinaldehyde (Compound 129)

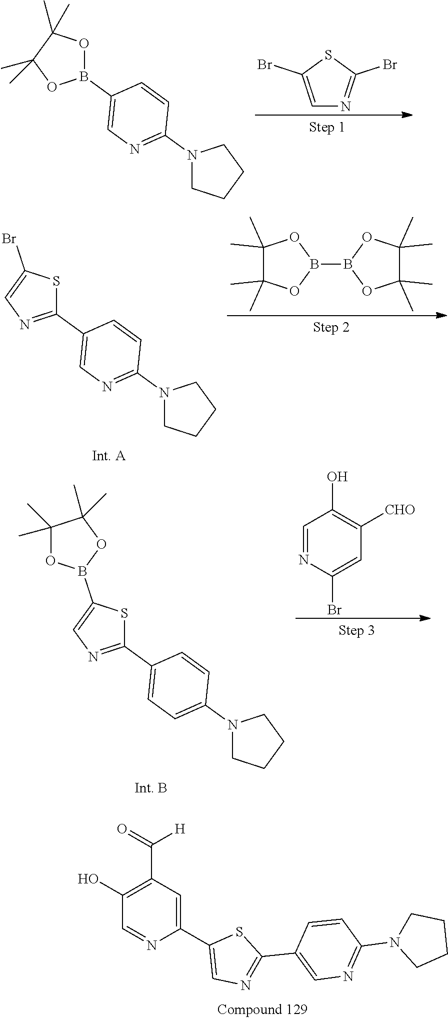

Step 1: Synthesis of 5-bromo-2-(6-(pyrrolidin-1-yl)pyridin-3-yl)thiazole: 5-bromo-2-(4-(pyrrolidin-1-yl)phenyl)thiazole, was accomplished by adding 2-(pyrrolidin-1-yl)-5-(3,3,4,4-tetramethylborolan-1-yl)pyridine (150 mg, 0.55 mmol), 2,5-dibromothiazole (199 mg, 0.82 mmol), G3-Pd Xantphos (25.9 mg, 0.03 mmol) and Xantphos (15.8 mg, 0.03 mmol) to a microwave vial. Dioxane (2.7 mL) and degassed 0.5M K₃PO₄ (2.7 mL) were added and the vial was degassed with argon. The sealed reaction vessel was heated to 140° C. in a microwave reactor for 1 hour. The mixture was cooled, diluted with dichloromethane (DCM) and extracted with saturated aqueous NaHCO₃. The combined aqueous layers were extracted 2 times more with DCM. The combined organic layers were washed with brine, dried over Na₂SO₄, filtered and evaporated. The residue was purified using 0-70% DCM/Hexanes to provide 5-bromo-2-(6-(pyrrolidin-1-yl)pyridin-3-yl)thiazole (69 mg, 41% yield). MS m/z [M+H]⁺ calc'd for C₁₂H₁₂BrN₃S, 310; found, 310.

Step 2: Synthesis of 2-(4-(pyrrolidin-1-yl)phenyl)-5-(4,4,5,5-tetramethyl-1,3,2-dioxaborolan-2-yl)thiazole: To a 5 mL microwave vial was added 5-bromo-2-(6-(pyrrolidin-1-yl)pyridin-3-yl)thiazole (329 mg, 1.5 mmol), bis(pinacolato)diboron (69 mg, 0.22 mmol), potassium acetate (65.5 mg, 0.67 mmol) and Pd(dppf)Cl₂ (8.1 mg) followed by 0.5 mL of dioxane. The mixture was purged with argon and heated in the microwave for 45 minutes at 140° C. The mixture was cooled, taken up in DCM filtered through celite and concentrated to dryness. The crude reaction mixture was used as is in the next reaction.

Step 3: To a 2 mL microwave vial was added 2-(4-(pyrrolidin-1-yl)phenyl)-5-(4,4,5,5-tetramethyl-1,3,2-dioxaborolan-2-yl)thiazole from Step 2, 2-bromo-5-hydroxyisonicotinaldehyde (45 mg, 0.22 mmol), XPhos Pd G3 (9.4 mg) and XPhos (5.3 mg) dioxane (0.9 mL) and degassed 0.5M K3PO4 (0.9 mL). The microwave vessel was purged with argon and heated to 130° C. in the microwave for 30 minutes. The reaction mixture was diluted with DCM, acidified to pH=3 with citric acid and extracted 3 times with DCM. The combined organic phases were washed with brine and dried over anhydrous Na₂SO₄, filtered and evaporated. The crude product was purified by PTLC 7% MeOH/DCM. Yield (19 mg, 24%). ¹H NMR (500 MHz, DMSO-d6) δ 11.31 (s, 1H), 10.39 (s, 1H), 8.68 (d, J=2.4 Hz, 1H), 8.45 (s, 1H), 8.37 (s, 1H), 8.02 (s, 1H), 8.01 (dd, J=8.9, 2.5 Hz, 1H), 6.56 (d, J=8.9 Hz, 1H), 3.47 (d, J=6.4 Hz, 4H), 2.08-1.90 (m, 4H). LC-MS m/z [M+H]⁺ calc'd for C₁₈H₁₆N₄O₂S, 353; found 353.

### Example 28: 3-fluoro-2-hydroxy-5-(3-(6-(pyrrolidin-1-yl)pyridin-3-yl)-1,2,4-thiadiazol-5-yl)benzaldehyde (Compound 130)

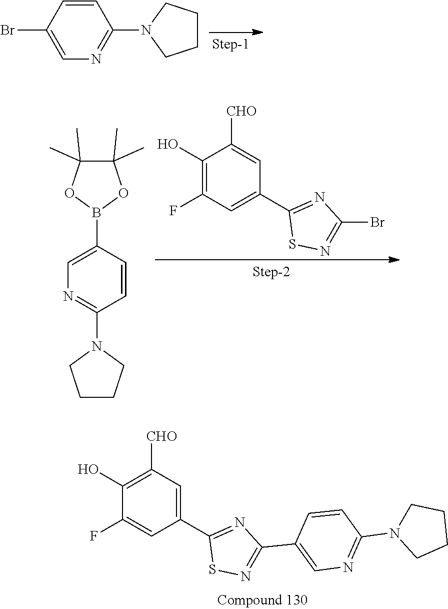

Step 1: Synthesis of 2-(pyrrolidin-1-yl)-5-(4,4,5,5-tetramethyl-1,3,2-dioxaborolan-2-yl)pyridine: 5-Bromo-2-(pyrrolidin-1-yl)pyridine (2 g, 8.8 mmol, 1.0 eq.) was dissolved in dioxane (30 mL). 4,4,4′,4′,5,5,5′,5′-Octamethyl-2,2′-bi(1,3,2-dioxaborolane) (3.37 g, 13.2 mmol, 1.5 eq.), potassium acetate (2.6 g, 26.4 mmol, 3.0 eq.), and PdCl₂(dppf) (430 mg, 0.5 mmol, 0.06 eq.) were added. The reaction was heated at 110° C. for 3 hours under N2 atmosphere. The solvent was removed in vacuo. The residue as suspended in water and extracted with ethyl acetate for three times. The organic extracts were combined, washed with brine, dried over anhydrous sodium sulfate, and concentrated. The residue was purified by silica gel column chromatography (petroleum ether/EtOAc=10:1 to 1:1) to give 2-(pyrrolidin-1-yl)-5-(4,4,5,5-tetramethyl-1,3,2-dioxaborolan-2-yl)pyridine (2.7 g, quantitative yield) as a yellow oil. LC-MS m/z [M+H]⁺ calc'd for C₁₅H₂₃BN₂O₂, 275; found, 275.

Step 2: 2-(Pyrrolidin-1-yl)-5-(4,4,5,5-tetramethyl-1,3,2-dioxaborolan-2-yl)pyridine (297 mg, 1.08 mmol, 1.5 eq) was mixed with 5-(3-bromo-1,2,4-thiadiazol-5-yl)-3-fluoro-2-hydroxybenzaldehyde (220 mg, 0.72 mmol, 1.0 eq.), potassium carbonate (300 mg, 2.17 mmol, 3.0 eq.), PdCl₂(dppf) (60 mg, 0.07 mmol, 0.1 eq.) in dioxane/water (10 mL/3 mL) was heated at 95° C. for 1 hour under N2 atmosphere. The mixture was cooled to room temperature and poured into water. Then pH of the system was adjusted to 3-4 and washed with ethyl acetate. The organic layer was discarded. The water phase was neutralized to pH 7-8 and extracted with ethyl acetate for three times. The organic extracts were combined, washed with brine, dried over anhydrous sodium sulfate, and concentrated. The residue was dissolve in THF and triturated with petroleum ether. The resulting precipitate was collected and treated with THF/petroleum ether again until clean. The precipitate was then collected and dried to give 3-fluoro-2-hydroxy-5-(3-(6-(pyrrolidin-1-yl)pyridin-3-yl)-1,2,4-thiadiazol-5-yl)benzaldehyde (60 mg, 0.16 mmol, 15% yield) as a yellow solid. ¹H NMR (DMSO-d6, 400 MHz) δ: 10.33 (s, 1H), 8.94 (s, 1H), 8.23 (dd, J=11.6 Hz, 2.4 Hz, 1H), 8.16 (dd, J=9.6 Hz, 2.0 Hz, 1H), 8.10 (s, 1H), 6.55 (d, J=9.2 Hz, 1H), 3.48 (m, 4H), 1.97 (m, 4H). LC-MS m/z [M+H]‘ calc’d for C₁₈H₁₅FN₄O₂S, 371; found, 371.

### Example 29: 2-fluoro-6-formyl-4-(3-(4-(pyrrolidin-1-yl)phenyl)-1,2,4-thiadiazol-5-yl)phenyl dihydrogen phosphate (Compound 16)

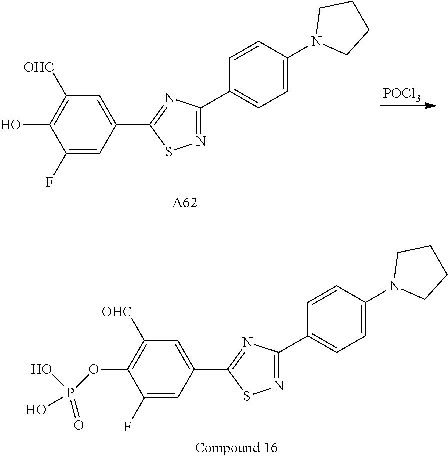

A solution of 3-fluoro-2-hydroxy-5-(3-(4-(pyrrolidin-1-yl)phenyl)-1,2,4-thiadiazol-5-yl)benzaldehyde [A62] (250 mg, 0.68 mmol, 1.0 eq.) and TEA (615 mg, 6.1 mmol, 9.0 eq.) in dichloromethane (10 mL) was cooled to 0° C. Then POCl₃ (228 mg, 1.5 mmol, 2.2 eq.) was added dropwise. The reaction was stirred for 30 min at room temperature. Then reaction mixture was poured into ice-water and pH of the system was adjusted 3-4 with KHSO₄. The mixture was extracted with dichloromethane for three times. The organic extracts were combined, dried over anhydrous sodium sulfate, and concentrated. The solid was dissolved in dichloromethane and 10% petroleum ether was added. Then the solution was concentrated until precipitation took place. The resulting precipitate was collected and dried to give 2-fluoro-6-formyl-4-(3-(4-(pyrrolidin-1-yl)phenyl)-1,2,4-thiadiazol-5-yl)phenyl dihydrogen phosphate (100 mg, 0.15 mmol, 56% yield) as a light yellow solid. ¹H NMR (DMSO-d6, 400 MHz) δ: 10.42 (br, 1H), 8.13 (m, 4H), 6.65 (m, 2H), 3.32 (m, 4H), 1.98 (m, 4H). ³¹P NMR (DMSO-d6, 162 MHz) δ: −9.58 (s, 1H).

### Example 30: 3-fluoro-5-formyl-4-hydroxy-N-(3-phenyl-1,2,4-thiadiazol-5-yl)benzamide (Compound 121)

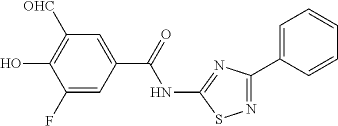 [SMILES: O=Cc1cc(C(=O)Nc2nc(-c3ccccc3)ns2)cc(F)c1O]

The title compound was prepared from 5-bromo-3-fluoro-2-hydroxybenzaldehyde (100 mg, 0.46 mmol, 1.6 eq.) and 3-phenyl-1,2,4-thiadiazol-5-amine (50 mg, 0.28 mmol, 1.0 eq.) using a method similar to that as described in Step 3 of Example 20. The desired product was obtained (14 mg, 8% yield). ¹H NMR (500 MHz, DMSO-d6) δ 15.56 (bs, 1H), 13.60 (bs, 1H), 10.53-10.19 (m, 1H), 8.41 (d, J=2.2 Hz, 1H), 8.25 (dd, J=11.8, 2.3 Hz, 1H), 8.23-8.19 (m, 2H), 7.57-7.50 (m, 3H). LC-MS m/z [M+H]⁺ calc'd for C₁₆H₁₀FN₃O₃S, 344. Found 344.

### Example 31: 3-fluoro-2-hydroxy-5-(4-(6-(pyrrolidin-1-yl)pyridin-3-yl)piperazine-1-carbonyl)benzaldehyde (Compound 140)

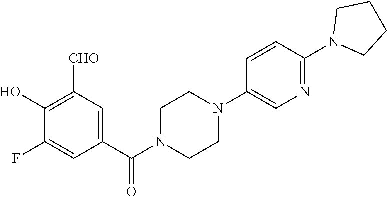 [SMILES: O=Cc1cc(C(=O)N2CCN(c3ccc(N4CCCC4)nc3)CC2)cc(F)c1O]

Step 1: In a 50 mL glass vial, Pd₂(dba)₃ (220 mg, 0.24 mmol, 0.1 eq.) was added to a mixture of 5-bromo-2-(pyrrolidin-1-yl)pyridine (550 mg, 2.43 mmol, 1 eq.), tert-butyl piperazine-1-carboxylate (905 mg, 4.87 mmol, 2 eq.), tBuONa (467 mg, 4.86 mmol, 2 eq.), and Xphos (139 mg, 0.24 mmol, 0.1 eq.) in toluene (10 mL). The reaction mixture was heated for 5 h at 100° C., then cooled to rt, poured into water, and extracted with ethyl acetate three times. The organic extracts were combined, washed with brine, dried over anhydrous sodium sulfate, and concentrated. The residue was purified by silica gel column chromatography (PE/EA=20:1 to 5:1) to give tert-butyl 4-(6-(pyrrolidin-1-yl)pyridin-3-yl)piperazine-1-carboxylate (210 mg, 0.63 mmol, 26% yield). LC-MS m/z [M+H]⁺ calc'd for C₁₈H₂₉N₄O₂ 333, Found 333.

Step 2: In a 50 mL glass vial, tert-butyl 4-(6-(pyrrolidin-1-yl)pyridin-3-yl)piperazine-1-carboxylate (210 mg, 0.63 mmol, 1 eq.) was treated with 8N HCl (gas)/Dioxane (5 mL) for 1 h at rt. The reaction mixture was concentrated in vacuo and co-evaporated with DCM two times to give crude 1-(6-(pyrrolidin-1-yl)pyridin-3-yl)piperazine dihydrochloride (161 mg, 0.53 mmol, 84% yield), which was used for next reaction without further purification. LC-MS m/z [M+H]⁺ calc'd for C₁₃H₂₁N₄ 233, found 233.

Step 3: In a 50 mL glass vial, HATU (309 mg, 0.81 mmol, 1.5 eq.) was added to a solution of 3-fluoro-5-formyl-4-hydroxybenzoic acid (146 mg, 0.54 mmol, 1 eq.), NMM (219 mg, 2.17 mmol, 4 eq.), and 1-(6-(pyrrolidin-1-yl)pyridin-3-yl)piperazine dihydrochloride (161 mg, 0.53 mmol, 1 eq.) in DCM (5 mL). The reaction was stirred overnight at rt. The solution was poured into water and extracted with ethyl acetate three times. The organic extracts were combined, washed with brine, dried over anhydrous sodium sulfate, and concentrated. The residue slurried in PE/DCM (10 mL, 5:1) and the filtrate was concentrated. The resulting residue was purified by silica gel column chromatography (PE/EA=5:1 to 1:1) and slurried in PE/DCM (5 mL, 10:1) to give 3-fluoro-2-hydroxy-5-(4-(6-(pyrrolidin-1-yl)pyridin-3-yl)piperazine-1-carbonyl)benzaldehyde (30 mg, 0.08 mmol, 14% yield) as a yellow solid. ¹H NMR (DMSO-d6, 400 MHz) δ: 10.29 (s, 1H), 7.79 (d, J=2.4 Hz, 1H), 7.63 (dd, J=10.8 Hz, 1.6 Hz, 1H), 7.55 (s, 1H), 7.32 (dd, J=8.4 Hz, 2.8 Hz, 1H), 6.42 (d, J=8.8 Hz, 1H), 3.63 (m, 4H), 3.31 (m, 4H), 2.96 (m, 4H), 1.91 (m, 4H). LC-MS m/z [M+H]⁺ calc'd for C₂₁H₂₄FN₄O₃ 399, found 399.

### Example 32: 3-fluoro-2-hydroxy-5-(4-(3-(pyrrolidin-1-yl)phenyl)-1,4-diazepane-1-carbonyl)benzaldehyde (Compound 98)

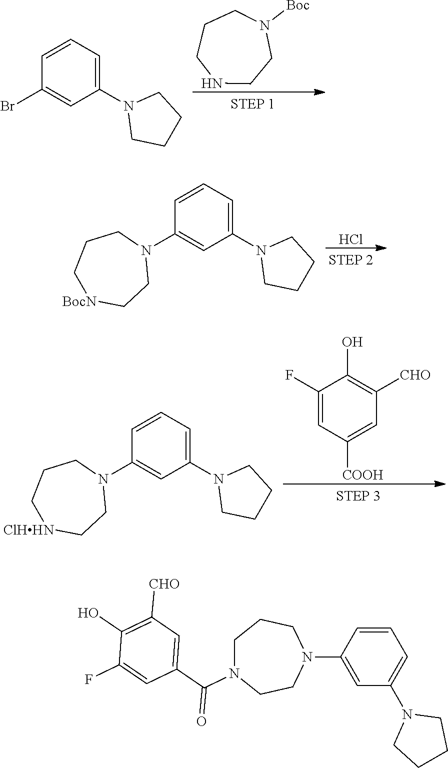

Step 1: In a 50 mL glass vial, Pd₂(dba)₃ (407 mg, 0.44 mmol, 0.1 eq.) was added to a mixture of 1-(3-bromophenyl)pyrrolidine (1 g, 4.44 mmol, 1 eq.), tert-butyl 1,4-diazepane-1-carboxylate (1.78 g, 8.88 mmol, 2 eq.), tBuONa (996 mg, 8.88 mmol, 2 eq.), and Xphos (257 mg, 0.44 mmol, 0.1 eq.) in toluene (50 mL). The reaction was heated for 5 h at 100° C. The reaction mixture was cooled to rt, poured into water, and extracted with ethyl acetate three times. The organic extracts were combined, washed with brine, dried over anhydrous sodium sulfate, and concentrated. The residue was purified by silica gel column chromatography (PE/EA=20:1 to 5:1) to give crude tert-butyl 4-(3-(pyrrolidin-1-yl)phenyl)-1,4-diazepane-1-carboxylate (1.3 g, 3.76 mmol, 85% yield), which was used for next reaction without further purification. LC-MS m/z [M+H]⁺ calc'd for C₂₀H₃₂N₃O₂, 346, found 346.

Step 2: In a 50 mL glass vial, tert-butyl 4-(3-(pyrrolidin-1-yl)phenyl)-1,4-diazepane-1-carboxylate (400 mg, 1.16 mmol, 1 eq.) was treated with 8N HCl (gas)/Dioxane (5 mL) for 1 h at rt. The reaction mixture was concentrated in vacuo and co-evaporated with DCM two times to give crude 1-(3-(pyrrolidin-1-yl)phenyl)-1,4-diazepane hydrochloride (327 mg, 1.16 mmol, quantitative yield), which was used for next reaction without further purification.

Step 3: In a 50 mL glass vial, HATU (661 mg, 1.74 mmol, 1.5 eq.) was added to a solution of 3-fluoro-5-formyl-4-hydroxybenzoic acid (235 mg, 1.28 mmol, 1.1 eq.), NMM (468 mg, 4.63 mmol, 4 eq.), and 1-(3-(pyrrolidin-1-yl)phenyl)-1,4-diazepane hydrochloride (327 mg, 1.16 mmol, 1 eq.) in DCM (5 mL). The reaction was stirred overnight at rt. The solution was poured into water and extracted with ethyl acetate three times. The organic extracts were combined, washed with brine, dried over anhydrous sodium sulfate, and concentrated. The residue was slurried in PE/DCM (10 mL, 5:1) and the filtrate was concentrated. The resulting residue was purified by silica gel column chromatography (PE/EA=5:1 to 1:1) and slurried in PE/DCM (5 mL, 10:1) to give 3-fluoro-2-hydroxy-5-(4-(3-(pyrrolidin-1-yl)phenyl)-1,4-diazepane-1-carbonyl)benzaldehyde (16 mg, 0.04 mmol, 3% yield) as a yellow solid. ¹H NMR (DMSO-d6, 400 MHz) δ: 11.17 (br, 1H), 10.10 (br, 1H), 6.93 (m, 2H), 5.93 (m, 1H), 5.82 (m, 1H), 5.43 (m, 1H), 3.62 (m, 8H), 2.92 (m, 4H), 1.84 (m, 6H). LC-MS m/z [M+H]⁺ calc'd for C₂₄H₂₆FN₃O₃ 412, found 412.

### Example 33: 3-fluoro-2-hydroxy-5-(4-(6-(pyrrolidin-1-yl)pyridin-3-yl)-1,4-diazepane-1-carbonyl)benzaldehyde (Compound 141)

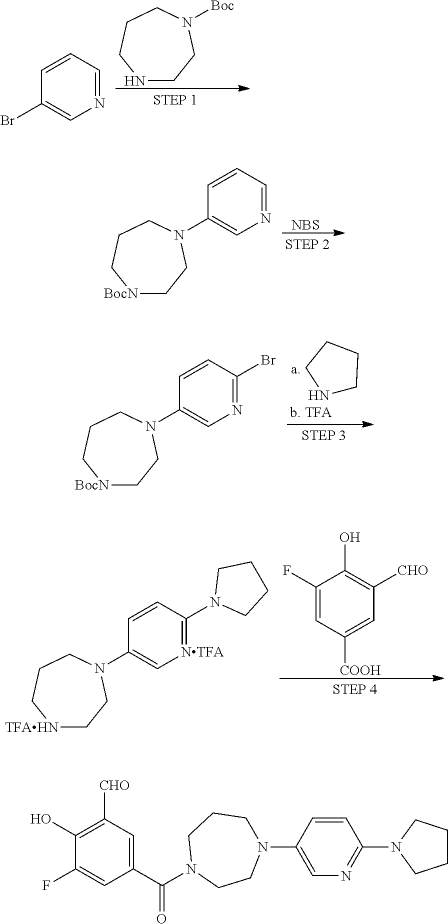

Step 1: In a 100 mL glass vial, Pd₂(dba)₃ (583 mg, 0.64 mmol, 0.1 eq.) was added to a mixture of 3-bromopyridine (1 g, 6.37 mmol, 1 eq.), tert-butyl 1,4-diazepane-1-carboxylate (1.27 g, 6.37 mmol, 1 eq.), tBuONa (1.2 g, 12.74 mmol, 2 eq.), and Xphos (367 mg, 0.64 mmol, 0.1 eq.) in toluene (50 mL). The reaction was heated for 5 h at 100° C. The reaction mixture was cooled to rt, acidified to pH 3-4, diluted with ethyl acetate, and filtered. The ethyl acetate phase was separated, neutralized to pH 7-8, and separated. The organic phase was dried over anhydrous sodium sulfate, and concentrated to give crude tert-butyl 4-(pyridin-3-yl)-1,4-diazepane-1-carboxylate (1.3 g, 4.69 mmol, 74% yield) as an oil, which was used for next reaction without further purification. LC-MS m/z [M+H]⁺ calc'd for C₁₅H₂₄N₃O₂278, found 278.

Step 2: NBS (752 mg, 4.22 mmol, 0.9 eq.) was added to a solution of tert-butyl 4-(pyridin-3-yl)-1,4-diazepane-1-carboxylate (1.3 g, 4.69 mmol, 1 eq.) in acetonitrile (20 mL). The reaction was stirred for 1 h at rt. The solution was poured into water and extracted with ethyl acetate three times. The organic extracts were combined, dried over anhydrous sodium sulfate, and concentrated. The residue was purified by silica gel column chromatography (PE/EA=10:1 to 5:1) to give tert-butyl 4-(6-bromopyridin-3-yl)-1,4-diazepane-1-carboxylate (1.6 g, 4.50 mmol, quantitative yield) as an oil, which was used for next reaction without further purification. LC-MS m/z [M+H]⁺ calc'd for C₁₅H₂₃BrN₃O₂ 356, Found 356.

Step 3a: In a 100 mL glass vial, Pd₂(dba)₃ (413 mg, 0.45 mmol, 0.1 eq.) was added to a mixture of tert-butyl 4-(6-bromopyridin-3-yl)-1,4-diazepane-1-carboxylate (1.6 g, 4.50 mmol, 1 eq.), pyrrolidine (640 mg, 9.00 mmol, 2 eq.), tBuONa (865 mg, 9.00 mmol, 2 eq.), and Xphos (260 mg, 0.45 mmol, 0.1 eq.) in toluene (50 mL). The reaction was heated for 5 h at 100° C. The reaction mixture was cooled to rt, acidified to pH 3-4, and washed with ethyl acetate. The water phase was separated, neutralized to pH 7-8, and extracted with ethyl acetate three times. The organic phases was combined, dried over anhydrous sodium sulfate, and concentrated. The residue was purified by silica gel column chromatography (PE/EA=10:1 to 2:1) to give tert-butyl 4-(6-(pyrrolidin-1-yl)pyridin-3-yl)-1,4-diazepane-1-carboxylate (1.1 g, 3.18 mmol, 71% yield) as a solid. LC-MS m/z [M+H]⁺ calc'd for C₁₉H₃₁N₄O₂ 347, found 347.

Step 3b: In a 50 mL glass vial, tert-butyl 4-(6-(pyrrolidin-1-yl)pyridin-3-yl)-1,4-diazepane-1-carboxylate (200 mg, 0.58 mmol, 1 eq.) was treated with TFA/DCM (1:3, 10 mL) for 1 h at rt. The reaction mixture was concentrated in vacuo and co-evaporated with DCM two times to give crude 1-(6-(pyrrolidin-1-yl)pyridin-3-yl)-1,4-diazepane·2TFA (283 mg, 0.58 mmol, quantitative yield), which was used for next reaction without further purification.

Step 4: In a 50 mL glass vial, EDCI (110 mg, 0.58 mmol, 1 eq.) was added to a solution of 3-fluoro-5-formyl-4-hydroxybenzoic acid (107 mg, 0.58 mmol, 1.1 eq.), TEA (176 mg, 1.74 mmol, 3 eq.), and 1-(6-(pyrrolidin-1-yl)pyridin-3-yl)-1,4-diazepane·2TFA (283 mg, 0.58 mmol, 1 eq.) in DCM (5 mL). The reaction was stirred overnight at rt. The solution was poured into water and extracted with ethyl acetate three times. The organic extracts were combined, washed with brine, dried over anhydrous sodium sulfate, and concentrated. The resulting residue was purified by prep HPLC to give 3-fluoro-2-hydroxy-5-(4-(6-(pyrrolidin-1-yl)pyridin-3-yl)-1,4-diazepane-1-carbonyl)benzaldehyde·TFA (35 mg, 0.07 mmol, 11% yield) as a yellow solid. ¹H NMR (DMSO-d6, 400 MHz) δ: 12.60 (br, 1H), 11.40 (br, 1H), 10.22 (s, 1H), 7.65 (d, J=8.4 Hz, 1H), 7.30 (d, J=10.8 Hz, 1H), 6.88 (m, 3H), 3.85-3.44 (m, 12H), 2.02 (m, 4H), 1.90 (m, 2H). LC-MS m/z [M+H]⁺ calc'd for C₂₄H₂₇F₄N₄O₅ 413, found 413.

### Example 34: 3-fluoro-2-hydroxy-5-(4-hydroxy-4-(6-(pyrrolidin-1-yl)pyridin-3-yl)piperidine-1-carbonyl)benzaldehyde (Compound 142)

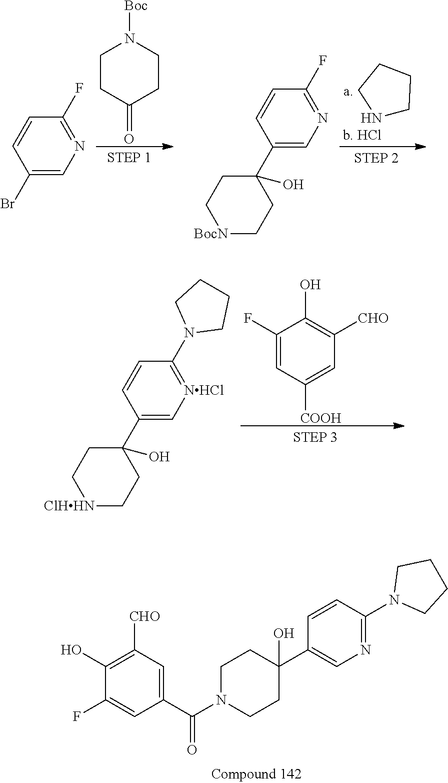

Step 1: BuLi (6.8 mL, 0.5 M in THF/hexane, 1.5 eq.) was added to a solution of 5-bromo-2-fluoropyridine (2 g, 11.4 mmol, 1 eq.) in diethyl ether (20 mL) at −78° C. under nitrogen protection. The reaction was stirred for 30 min at −78° C. and a solution of tert-butyl 4-oxopiperidine-1-carboxylate (2.26 g, 11.4 mmol, 1 eq.) in THF (5 mL) was added dropwise. The cooling system was removed and the reaction was stirred for another 40 min. The mixture was poured into sat. NH₄Cl solution and extracted with ethyl acetate three times. The organic extracts were combined, washed with brine, dried over anhydrous sodium sulfate, and concentrated. The residue was purified by silica gel column chromatography (PE/EA=20:1 to 5:1) to give tert-butyl 4-(6-fluoropyridin-3-yl)-4-hydroxypiperidine-1-carboxylate (1.3 g, 4.39 mmol, 39% yield) as a yellow oil. LC-MS m/z [M+H]⁺ calc'd for C₁₅H₂₂FN₂O₃ 297.3, found 297.

Step 2a: A solution of tert-butyl 4-(6-fluoropyridin-3-yl)-4-hydroxypiperidine-1-carboxylate (1.3 g, 4.39 mmol, 1 eq.) and pyrrolidine (1.25 g, 17.56 mmol, 4 eq.) in MeOH (30 mL) was refluxed overnight. The solvent was removed in vacuo. The residue was suspended in water and extracted with ethyl acetate three times. The organic extracts were combined, dried over anhydrous sodium sulfate, and concentrated. The residue was purified by silica gel column chromatography (PE/acetone=10:1 to 5:1) to give tert-butyl 4-hydroxy-4-(6-(pyrrolidin-1-yl)pyridin-3-yl)piperidine-1-carboxylate (380 mg, 1.10 mmol, 23% yield) as a yellow oil. LC-MS m/z [M+H]⁺ calc'd for C₁₉H₃₀N₃O₃ 348, found 348.

Step 2b: In a 50 mL glass vial, tert-butyl 4-hydroxy-4-(6-(pyrrolidin-1-yl)pyridin-3-yl)piperidine-1-carboxylate (200 mg, 0.58 mmol, 1 eq.) was treated with TFA/DCM (1:3, 10 mL) for 1 h at rt. The reaction mixture was concentrated in vacuo and co-evaporated with DCM two times to give crude 4-(6-(pyrrolidin-1-yl)pyridin-3-yl)piperidin-4-ol·2TFA (285 mg, 0.58 mmol, quantitative yield), which was used for next reaction without further purification.

Step 3: In a 50 mL glass vial, EDCI (110 mg, 0.58 mmol, 1 eq.) was added to a solution of 3-fluoro-5-formyl-4-hydroxybenzoic acid (107 mg, 0.58 mmol, 1.1 eq.), NMM (234 mg, 2.32 mmol, 4 eq.), and 4-(6-(pyrrolidin-1-yl)pyridin-3-yl)piperidin-4-ol·2TFA (285 mg, 0.58 mmol, 1 eq.) in DCM (5 mL). The reaction was stirred overnight at rt. The solution was poured into water and extracted with ethyl acetate three times. The organic extracts were combined, washed with brine, dried over anhydrous sodium sulfate, and concentrated. The resulting residue was purified by prep HPLC to give 3-fluoro-2-hydroxy-5-(4-hydroxy-4-(6-(pyrrolidin-1-yl)pyridin-3-yl)piperidine-1-carbonyl)benzaldehyde·TFA (6 mg, 0.01 mmol, 3% yield) as a white solid. ¹H NMR (DMSO-d6, 400 MHz) δ: 11.41 (br, 1H), 10.31 (s, 1H), 8.18 (d, J=8.8 Hz, 1H), 7.85 (s, 1H), 7.67 (dd, J=11.2 Hz, 2.0 Hz, 1H), 7.59 (m, 2H), 7.10 (d, J=9.6 Hz, 1H), 3.44 (m, 8H), 2.01 (m, 6H), 1.59 (m, 2H). LC-MS m/z [M+H]⁺ calc'd for C₂₂H₂₅FN₃O₄, 414.0, found 414.

### Example 35: 3-fluoro-5-formyl-4-hydroxy-N-(4-(pyrrolidin-1-yl)phenethyl)benzamide (Compound 143)

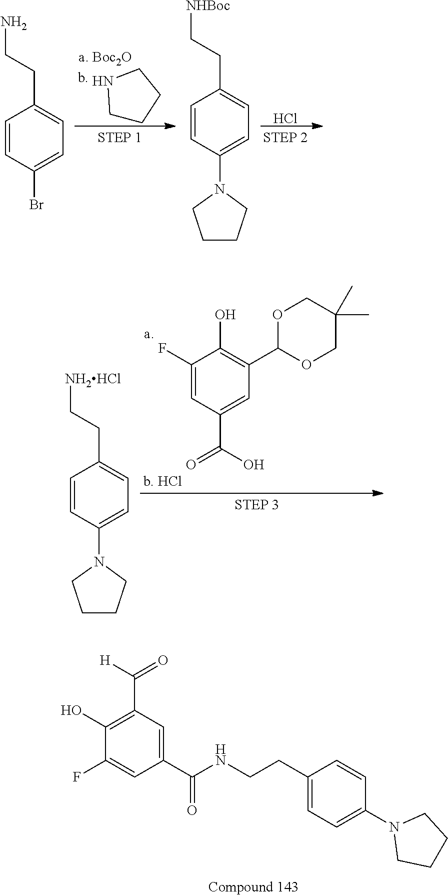

Step 1a: In a 100 mL glass vial, Boc₂O (1.2 g, 5.50 mmol, 1.1 eq.) was added to a mixture of 2-(4-bromophenyl)ethanamine (1 g, 5.03 mmol, 1 eq.) and NaHCO₃ (1.27 g, 15.0 mmol, 3 eq.), in THF/water (5 mL/5 mL). The reaction was stirred for 2 h at rt. The solution was extracted with ethyl acetate three times. The organic extracts were combined, washed with brine, dried over anhydrous sodium sulfate, and concentrated to give crude tert-butyl 4-bromophenethylcarbamate (1.53 g, 5.03 mmol, quantitative yield), which was used for next reaction without further purification.

Step 1b: In a 100 mL glass vial, Pd₂(dba)₃ (245 mg, 0.27 mmol, 0.1 eq.) was added to a mixture of tert-butyl 4-bromophenethylcarbamate (800 mg, 2.68 mmol, 1 eq.), pyrrolidine (380 mg, 5.36 mmol, 2 eq.), Cs₂CO₃ (1.74 g, 5.36 mmol, 2 eq.), and Xphos (155 mg, 0.27 mmol, 0.1 eq.) in toluene (30 mL) was heated for 2 h at 95° C. The reaction mixture was cooled to rt, poured into water, and extracted with ethyl acetate. The organic phase was dried over anhydrous sodium sulfate, and concentrated. The residue was purified by silica gel column chromatography (PE/EA=50:1 to 20:1) to give tert-butyl 4-(pyrrolidin-1-yl)phenethylcarbamate (210 mg, 0.72 mmol, 27% yield). Pos. LC-MS: 291.2 (M+H)+, C₁₇H₂₆N₂O₂.

Step 2: In a 50 mL glass vial, tert-butyl 3-(4-(pyrrolidin-1-yl)phenyl)propylcarbamate (200 mg, 0.69 mmol, 1 eq.) was treated with 8N HCl(gas)/dioxane (5 mL) for 1 h at rt. The reaction mixture was concentrated in vacuo and co-evaporated with DCM two times to give crude 2-(4-(pyrrolidin-1-yl)phenyl)ethanamine hydrochloride (163 mg, 0.69 mmol, quantitative yield), which was used for next reaction without further purification. No LCMS was taken for it.

Step 3a: In a 50 mL glass vial, HATU (262 mg, 0.69 mmol, 1.3 eq.) was added to a solution of 3-fluoro-5-formyl-4-hydroxybenzoic acid (171 mg, 0.63 mmol, 1.2 eq.), NMM (268 mg, 2.65 mmol, 5 eq.), and 2-(4-(pyrrolidin-1-yl)phenyl)ethanamine hydrochloride (120 mg, 0.53 mmol, 1 eq.) in DCM (5 mL). The reaction was stirred overnight at rt. The solution was poured into water and extracted with ethyl acetate three times. The organic extracts were combined, washed with brine, dried over anhydrous sodium sulfate, and concentrated. The resulting residue was purified by silica gel column chromatography (PE/EA/DCM=10:1 to 2:1:0.2) to give 3-(5,5-dimethyl-1,3-dioxan-2-yl)-5-fluoro-4-hydroxy-N-(4-(pyrrolidin-1-yl)phenethyl)benzamide (95 mg, 0.21 mmol, 41% yield). LC-MS m/z [M+H]⁺ calc'd for C₂₅H₃₂FN₂O₄, 443, found 443.

Step 3b: 3-(5,5-Dimethyl-1,3-dioxan-2-yl)-5-fluoro-4-hydroxy-N-(4-(pyrrolidin-1-yl)phenethyl)benzamide (95 mg, 0.21 mmol, 1 eq.) was treated with 4N HCl/THF (10 mL/10 mL) for 2 h. The solution was neutralized with sodium bicarbonate to pH 7-8 and extracted with ethyl acetate three times. The organic extracts were combined, washed with brine, dried over anhydrous sodium sulfate, and concentrated. The residue was purified by silica gel column chromatography and prep HPLC to give 3-fluoro-5-formyl-4-hydroxy-N-(4-(pyrrolidin-1-yl)phenethyl)benzamide (18 mg, 0.05 mmol, 24% yield) as a white solid. ¹H NMR (CDCl₃, 400 MHz) δ: 11.22 (s, 1H), 9.95 (d, J=1.6 Hz, 1H), 7.83 (s, 1H), 7.68 (dd, J=11.2 Hz, 2.0 Hz, 1H), 7.15 (d, J=8.4 Hz, 2H), 6.83 (m, 2H), 6.14 (m, 1H), 3.67 (m, 2H), 3.38 (m, 4H), 2.87 (m, 2H), 2.09 (m, 4H). LC-MS m/z [M+H]⁺ calc'd for C₂₀H₂₂FN₂O₃, 357, Found 357.

### Example 36: 3-fluoro-5-formyl-4-hydroxy-N-(3-(4-(pyrrolidin-1-yl)phenyl)propyl)benzamide (Compound 144)

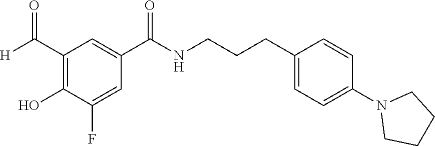 [SMILES: O=Cc1cc(C(=O)NCCCc2ccc(N3CCCC3)cc2)cc(F)c1O]

3-fluoro-5-formyl-4-hydroxy-N-(3-(4-(pyrrolidin-1-yl)phenyl)propyl)benzamide was prepared using a method similar to that as described in Example 35 starting from 3-(4-bromophenyl)propan-1-amine. ¹H NMR (DMSO-d6, 400 MHz) δ: 11.52 (br, 1H), 10.31 (s, 1H), 8.53 (s, 1H), 8.05 (s, 1H), 7.94 (d, J=11.6 Hz, 1H), 7.00 (d, J=7.6 Hz, 2H), 6.45 (d, J=7.6 Hz, 2H), 3.23 (m, 2H), 3.17 (m, 4H), 1.93 (m, 4H), 1.75 (m, 2H). LC-MS m/z [M+H]⁺ calc'd for C₂₁H₂₄FN₂O₃ 371, found 371.

### Example 37: 3-fluoro-2-hydroxy-5-(4-(6-(pyrrolidin-1-yl)pyridin-3-yl)piperidine-1-carbonyl)benzaldehyde (Compound 37)

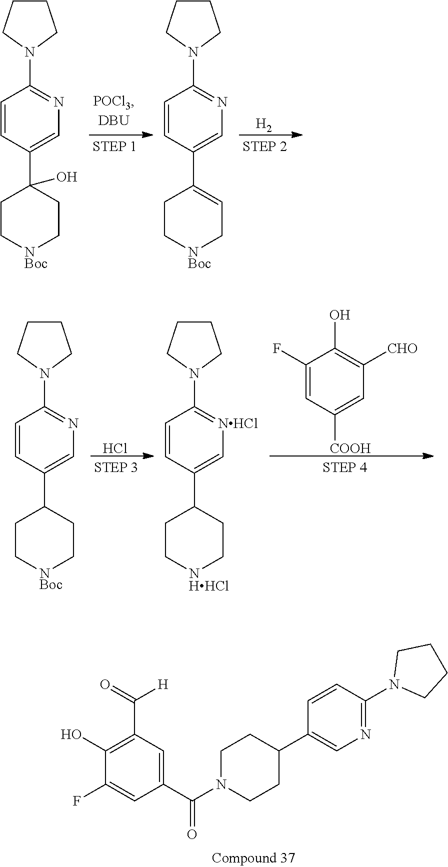

Step 1: POCl₃ (264 mg, 1.73 mmol, 2 eq.) was added dropwise to a solution of tert-butyl 4-hydroxy-4-(6-(pyrrolidin-1-yl)pyridin-3-yl)piperidine-1-carboxylate (300 mg, 0.86 mmol, 1 eq.) and DBU (263 mg, 1.73 mmol, 2 eq.) in pyridine (3 mL) at 0° C. The reaction was stirred at 80° C. for 2 h. The solution was cooled to rt, poured into water, and extracted with ethyl acetate three times. The organic extracts were combined, washed with brine, dried over anhydrous sodium sulfate, and concentrated. The residue was slurried in PE and filtered to give crude tert-butyl 4-(6-(pyrrolidin-1-yl)pyridin-3-yl)-5,6-dihydropyridine-1(2H)-carboxylate (250 mg, 0.76 mmol, 88% yield) as a solid.

Step 2: A solution of tert-butyl 4-(6-(pyrrolidin-1-yl)pyridin-3-yl)-5,6-dihydropyridine-1(2H)-carboxylate (250 mg, 0.76 mmol, 1 eq.) in MeOH (10 mL) was hydrogenated with Pd/C (100 mg) for 2 h at rt. Pd/C was filtered off and the filtration was concentrated in vacuo to give tert-butyl 4-(6-(pyrrolidin-1-yl)pyridin-3-yl)piperidine-1-carboxylate (230 mg, 0.69 mmol, 91% yield). Pos. LC-MS: 332.2 (M+H)+, C₁₉H₂₉N₃O₂.

Step 3: In a 50 mL glass vial, tert-butyl 4-(6-(pyrrolidin-1-yl)pyridin-3-yl)piperidine-1-carboxylate (230 mg, 0.69 mmol, 1 eq.) was treated with 8 N HCl(gas)/dioxane (10 mL) for 1 h at rt. The reaction mixture was concentrated in vacuo and co-evaporated with DCM two times to give crude 5-(piperidin-4-yl)-2-(pyrrolidin-1-yl)pyridine dihydrochloride (223 mg, 0.69 mmol, quantitative yield), which was used for next reaction without further purification. Pos. LC-MS: 231.9 (M+H)⁺, C₁₄H₂₃Cl₂N₃.

Step 4: In a 50 mL glass vial, EDCI (69 mg, 0.36 mmol, 1.2 eq.) was added to a solution of 3-fluoro-5-formyl-4-hydroxybenzoic acid (60 mg, 0.33 mmol, 1.1 eq.), TEA (91 mg, 0.90 mmol, 3 eq.), and 5-(piperidin-4-yl)-2-(pyrrolidin-1-yl)pyridine dihydrochloride (90 mg, 0.30 mmol, 1 eq.) in DCM (5 mL). The reaction was stirred overnight at 30° C. The solution was poured into water and extracted with DCM three times. The organic extracts were combined and concentrated in vacuo. The residue was suspended in water and pH was adjusted to 3-4. The mixture was washed with ethyl acetate three times. The water phase was neutralized to pH 7-8 and extracted with ethyl acetate three times and DCM three times. The organic extracts were combined, dried over anhydrous sodium sulfate, and filtered through a short pad of silica gel. The filtrate was concentrated to give 3-fluoro-2-hydroxy-5-(4-(6-(pyrrolidin-1-yl)pyridin-3-yl)piperidine-1-carbonyl)benzaldehyde (44 mg, 0.11 mmol, 37% yield) as an off-white solid. ¹H NMR (CDCl₃, 400 MHz) δ: 9.96 (s, 1H), 8.01 (d, J=1.6 Hz, 1H), 7.57 (s, 1H), 7.47 (dd, J=10.8 Hz, 2.0 Hz, 1H), 7.32 (dd, J=8.8 Hz, 2.4 Hz, 1H), 6.37 (d, J=8.8 Hz, 1H), 4.78 (m, 1H), 3.94 (m, 1H), 3.45 (m, 4H), 3.06 (m, 2H), 2.70 (m, 1H), 2.01 (m, 4H), 1.87 (m, 2H), 1.64 (m, 2H). LC-MS m/z [M+H]⁺ calc'd for C₂₂H₂₅FN₃O₃ 398, found 398.

### Example 38: 3-fluoro-2-hydroxy-5-(4-(2-(pyrrolidin-1-yl)pyridin-4-yl)piperidine-1-carbonyl)benzaldehyde (Compound 36)

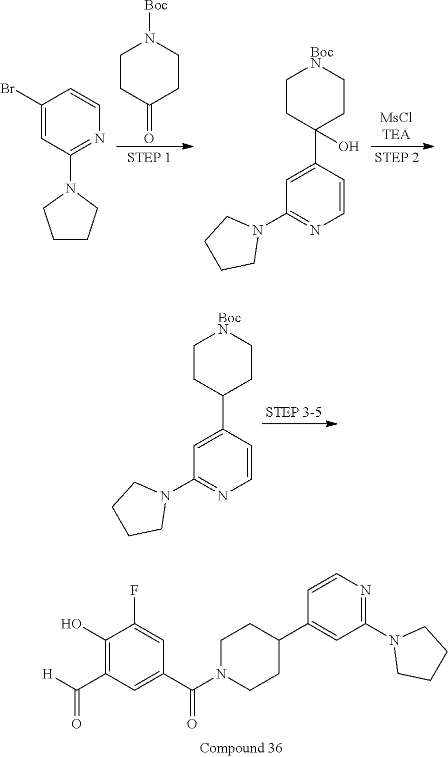

Step 1: BuLi (2.7 mL, 2.5 M in THF/hexane, 6.64 mmol, 1.5 eq.) and a solution of tert-butyl 4-oxopiperidine-1-carboxylate (880 mg, 4.42 mmol, 1 eq.) in THF (5 mL) were added dropwise. The cooling system was removed and the reaction was stirred for another 40 min. The mixture was poured into sat. NH₄Cl solution and extracted with ethyl acetate three times. The organic extracts were combined, washed with brine, dried over anhydrous sodium sulfate, and concentrated. The residue was purified by silica gel column chromatography (PE/EA=10:1 to 1:4) to give tert-butyl 4-hydroxy-4-(2-(pyrrolidin-1-yl)pyridin-4-yl)piperidine-1-carboxylate (1.02 g, 2.94 mmol, 67% yield).). LC-MS m/z [M+H]⁺ calc'd for ⁺C₁₉H₃₀N₃O₃ 348, found 348.

Step 2: MsCl (989 mg, 8.64 mmol, 3 eq.) was added dropwise to a solution of tert-butyl 4-hydroxy-4-(2-(pyrrolidin-1-yl)pyridin-4-yl)piperidine-1-carboxylate (1 g, 2.88 mmol, 1 eq.) and TEA (1.45 g, 14.41 mmol, 5 eq.) in DCM (20 mL) at 0° C. The reaction was stirred overnight at rt. The solution was poured into water and extracted with ethyl acetate three times. The organic extracts were combined, washed with brine, dried over anhydrous sodium sulfate, and concentrated. The residue was purified by silica gel column chromatography (PE/EA=10:1 to 3:1) to give tert-butyl 4-(2-(pyrrolidin-1-yl)pyridin-4-yl)-5,6-dihydropyridine-1(2H)-carboxylate (701 mg, 2.13 mmol, 74% yield).). LC-MS m/z [M+H]⁺ calc'd for C₁₉H₂₇N₃O₂ 330, found 330.

Steps 3-5: The a similar procedure was followed as described for Example 37 to give 3-fluoro-2-hydroxy-5-(4-(2-(pyrrolidin-1-yl)pyridin-4-yl)piperidine-1-carbonyl)benzaldehyde. ¹H NMR (CDCl₃, 400 MHz) δ: 9.97 (s, 1H), 8.08 (d, J=5.2 Hz, 1H), 7.57 (s, 1H), 7.47 (dd, J=10.4 Hz, 2.0 Hz, 1H), 4.80 (m, 1H), 4.07 (m, 1H), 3.47 (m, 4H), 3.09 (m, 2H), 2.72 (m, 1H), 2.01 (m, 4H), 1.93 (m, 2H), 1.70 (m, 2H).). LC-MS m/z [M+H]⁺ calc'd for C₂₂H₂₄FN₃O₃ 398, found 398.

### Example 39: 3-fluoro-2-hydroxy-5-(4-(5-(pyrrolidin-1-yl)pyridin-2-yl)piperidine-1-carbonyl)benzaldehyde (Compound 145)

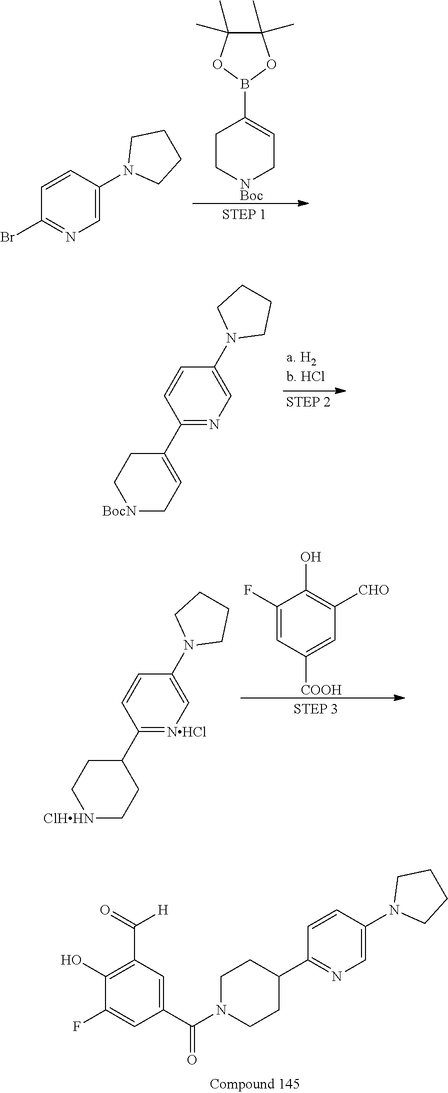

Step 1: In a 100 mL glass vial, Pd(dppf)₂Cl₂ (180 mg, 0.22 mmol, 0.1 eq.) was added to a mixture of 2-bromo-5-(pyrrolidin-1-yl)pyridine (500 mg, 2.21 mmol, 1 eq.), tert-butyl 4-(4,4,5,5-tetramethyl-1,3,2-dioxaborolan-2-yl)-5,6-dihydropyridine-1(2H)-carboxylate (750 mg, 2.43 mmol, 1.1 eq.), Na₂CO₃ (586 mg, 5.53 mmol, 2.5 eq.) in Dioxane/water (18 mL/6 mL). The reaction was heated overnight at 95° C. The reaction mixture was cooled to rt, poured into water, and extracted with ethyl acetate. The organic phase was dried over anhydrous sodium sulfate, and concentrated. The residue was purified by silica gel column chromatography (PE/EA/DCM=50:1:1 to 5:1:1) to give tert-butyl 4-(5-(pyrrolidin-1-yl)pyridin-2-yl)-5,6-dihydropyridine-1(2H)-carboxylate (650 mg, 1.98 mmol, 89% yield). Pos. LC-MS: 330.0 (M+H)⁺, C₁₉H₂₇N₃O₂.

Step 2a: A solution of tert-butyl 4-(5-(pyrrolidin-1-yl)pyridin-2-yl)-5,6-dihydropyridine-1(2H)-carboxylate (650 mg, 1.98 mmol, 1 eq.) in MeOH (10 mL) was hydrogenated with Pd/C (200 mg) for 2 h at rt. Pd/C was filtered off and the filtration was concentrated in vacuo to give tert-butyl 4-(5-(pyrrolidin-1-yl)pyridin-2-yl)piperidine-1-carboxylate (650 mg, 1.98 mmol, quantitative yield). LC-MS m/z [M+H]⁺ calc'd for C₁₉H₃₀N₃O₂ 332, found 332.

Step 2b: In a 50 mL glass vial, tert-butyl 4-(5-(pyrrolidin-1-yl)pyridin-2-yl)piperidine-1-carboxylate (650 mg, 1.98 mmol, 1 eq.) was treated with 8 N HCl(gas)/dioxane (15 mL) for 2 h at rt. The reaction mixture was concentrated in vacuo and co-evaporated with DCM two times to give crude 2-(piperidin-4-yl)-5-(pyrrolidin-1-yl)pyridine dihydrochloride (603 mg, 1.98 mmol, quantitative yield), which was used for next reaction without further purification.

Step 3: In a 50 mL glass vial, EDCI (128 mg, 0.67 mmol, 1.2 eq.) was added to a solution of 3-fluoro-5-formyl-4-hydroxybenzoic acid (114 mg, 0.62 mmol, 1.1 eq.), TEA (283 mg, 2.80 mmol, 5 eq.), and 5-(piperidin-4-yl)-2-(pyrrolidin-1-yl)pyridine dihydrochloride (171 mg, 0.56 mmol, 1 eq.) in DCM (5 mL). The reaction was stirred overnight at rt. The solution was poured into water and extracted with DCM three times. The organic extracts were combined and concentrated in vacuo. The residue was suspended in water and pH was adjusted to 3-4. The mixture was washed with ethyl acetate three times. The water phase was neutralized to pH 7-8 and extracted with ethyl acetate three times. The organic extracts were combined, dried over anhydrous sodium sulfate, and filtered through a short pad of silica gel. The filtrate was concentrated to give 3-fluoro-2-hydroxy-5-(4-(5-(pyrrolidin-1-yl)pyridin-2-yl)piperidine-1-carbonyl)benzaldehyde (40 mg, 0.10 mmol, 18% yield) as a white solid. ¹H NMR (CDCl₃, 400 MHz) δ: 9.96 (s, 1H), 7.92 (s, 1H), 7.57 (s, 1H), 7.48 (dd, J=10.8 Hz, 1.6 Hz, 1H), 7.01 (d, J=8.4 Hz, 1H), 6.82 (m, 1H), 4.74 (m, 1H), 3.92 (m, 1H), 3.29 (m, 4H), 3.17-2.86 (m, 3H), 2.02-1.93 (m, 6H), 1.81 (m, 2H). LC-MS m/z [M+H]⁺ calc'd for C₂₂H₂₄FN₃O₃ 398, found 398.

### Example 40: Synthesis of 3-fluoro-2-hydroxy-5-(4-(6-(pyrrolidin-1-yl)pyridin-2-yl)piperidine-1-carbonyl)benzaldehyde (Compound 146)

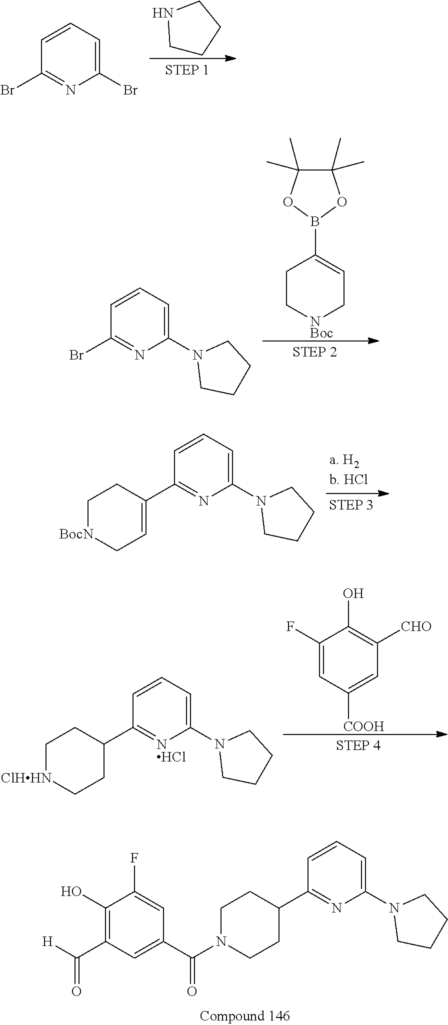

Step 1: A mixture of 2,6-dibromopyridine (500 mg, 2.11 mmol, 1 eq.), pyrrolidine (165 mg, 2.32 mmol, 1.1 eq.), and K₂CO₃ (915 mg, 6.63 mmol, 3 eq.) in DMF (10 mL) was heated overnight at 80° C. The reaction mixture was cooled to rt, poured into water, and extracted with ethyl acetate. The organic phase was washed with brine, dried over anhydrous sodium sulfate, and concentrated to give crude 2-bromo-6-(pyrrolidin-1-yl)pyridine (620 mg, 2.11 mmol, quantitative yield), which was used for next reaction without further purification. LC-MS m/z [M+H]⁺ calc'd for C₉H₁₂BrN₂ 227, found 227.

Step 2: In a 100 mL glass vial, Pd(dppf)₂Cl₂ (185 mg, 0.23 mmol, 0.1 eq.) was added to a mixture of 2-bromo-5-(pyrrolidin-1-yl)pyridine (512 mg, 2.27 mmol, 1 eq.), tert-butyl 4-(4,4,5,5-tetramethyl-1,3,2-dioxaborolan-2-yl)-5,6-dihydropyridine-1(2H)-carboxylate (840 mg, 2.72 mmol, 1.2 eq.), Na₂CO₃ (722 mg, 6.81 mmol, 3 eq.) in dioxane/water (18 mL/6 mL). The reaction was heated at 95° C. for 2 h. The reaction mixture was cooled to rt, poured into water, and extracted with ethyl acetate. The organic phase was dried over anhydrous sodium sulfate, and concentrated. The residue was purified by silica gel column chromatography (PE/EA/DCM=50:1:1 to 30:1:1) to give tert-butyl 4-(6-(pyrrolidin-1-yl)pyridin-2-yl)-5,6-dihydropyridine-1(2H)-carboxylate (503 mg, 1.53 mmol, 67% yield). LC-MS m/z [M+H]⁺ calc'd for C₁₉H₂₈N₃O₂ 330, found 330.

Step 3a: A solution of tert-butyl 4-(6-(pyrrolidin-1-yl)pyridin-2-yl)-5,6-dihydropyridine-1(2H)-carboxylate (503 mg, 1.53 mmol, 1 eq.) in MeOH (10 mL) was hydrogenated with Pd/C (200 mg) for 2 h at rt. Pd/C was filtered off and the filtration was concentrated in vacuo to give tert-butyl 4-(6-(pyrrolidin-1-yl)pyridin-2-yl)piperidine-1-carboxylate (510 mg, 1.53 mmol, quantitative yield). LC-MS m/z [M+H]⁺ calc'd for C₁₉H₃₀N₃O₂ 332, found 332.

Step 3b: In a 50 mL glass vial, tert-butyl 4-(6-(pyrrolidin-1-yl)pyridin-2-yl)piperidine-1-carboxylate (280 mg, 0.85 mmol, 1 eq.) was treated with 8 N HCl(gas)/dioxane (10 mL) for 2 h at rt. The reaction mixture was concentrated in vacuo and co-evaporated with DCM two times to give crude 2-(piperidin-4-yl)-6-(pyrrolidin-1-yl)pyridine dihydrochloride (263 mg, 0.85 mmol, quantitative yield), which was used for next reaction without further purification.

Step 4: In a 50 mL glass vial, EDCI (195 mg, 1.02 mmol, 1.2 eq.) was added to a solution of 3-fluoro-5-formyl-4-hydroxybenzoic acid (188 mg, 1.02 mmol, 1.2 eq.), TEA (429 mg, 4.25 mmol, 5 eq.), and 5-(piperidin-4-yl)-2-(pyrrolidin-1-yl)pyridine dihydrochloride (263 mg, 0.85 mmol, 1 eq.) in DCM (5 mL). The reaction was stirred overnight at rt. The solution was poured into water and extracted with DCM three times. The organic extracts were combined and concentrated in vacuo. The residue was suspended in water and pH was adjusted to 3-4. The mixture was washed with ethyl acetate three times. The water phase was neutralized to pH 7-8 and extracted with ethyl acetate three times. The organic extracts were combined, dried over anhydrous sodium sulfate, and filtered through a short pad of silica gel. The filtrate was concentrated to give 3-fluoro-2-hydroxy-5-(4-(6-(pyrrolidin-1-yl)pyridin-2-yl)piperidine-1-carbonyl)benzaldehyde (90 mg, 0.23 mmol, 27% yield) as a white solid. ¹H NMR (CDCl₃, 400 MHz) δ: 11.13 (br, 1H), 9.94 (s, 1H), 7.56 (s, 1H), 7.48 (dd, J=10.4 Hz, 2.0 Hz, 1H), 7.37 (m, 1H), 6.37 (d, J=7.2 Hz, 1H), 6.20 (d, J=8.4 Hz, 1H), 4.72 (m, 1H), 3.90 (m, 1H), 3.45 (m, 4H), 3.18 (m, 2H), 2.82 (m, 1H), 2.23-1.75 (m, 8H). LC-MS m/z [M+H]⁺ calc'd for C₂₂H₂₅FN₃O₃ 398, found 398.

### Example 41: 3-fluoro-2-hydroxy-5-(2-(4-(pyrrolidin-1-yl)phenyl)morpholine-4-carbonyl)benzaldehyde (Compound 147)

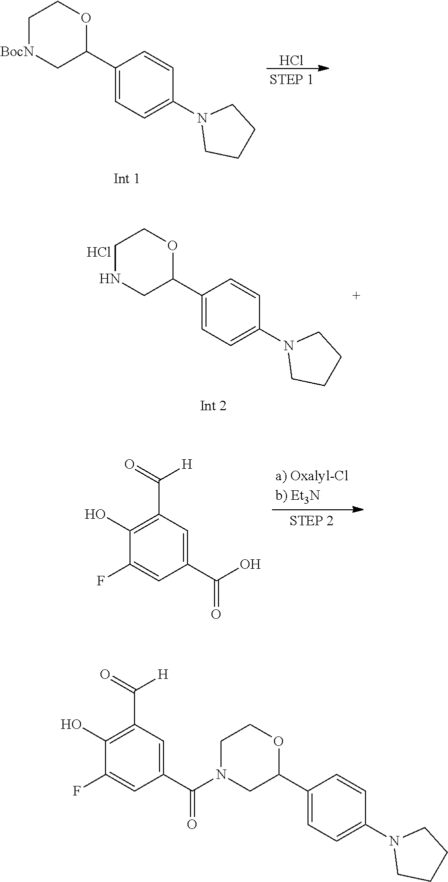 [SMILES: CC(C)(C)OC(=O)N1CCOC(c2ccc(N3CCCC3)cc2)C1 ; Cl ; Cl ; c1cc(N2CCCC2)ccc1C1CNCCO1 ; O=Cc1cc(C(=O)O)cc(F)c1O ; O=Cc1cc(C(=O)N2CCOC(c3ccc(N4CCCC4)cc3)C2)cc(F)c1O]

Step 1: Intermediate 1 was synthesized from 2-(4-bromophenyl)morpholine as described for Example 18. Tert-butyl 2-(4-bromophenyl)morpholine-4-carboxylate (79.4 mg, 0.24 mmol) was dissolved in 0.5 mL of DCM and 0.5 mL of 4N HCl in dioxane was added. The reaction mixture was stirred for 3 hours at room temperature. The solvent was removed in vacuo an the residue, Int 2, was used as is in the next reaction.

Step 2a: Oxalyl chloride (0.97 mL of 2M in DCM, 1.94 mmol, 1.2 eq.) and DMF (13.2 uL) were added to a solution of 3-fluoro-5-formyl-4-methoxybenzoic acid (307 mg, 1.62 mmol, 1.0 eq.) in DCM (3.2 mL) at 0° C. under argon. The reaction was allowed to warm to room temperature and stirred for 3 hours. The solvent was removed in vacuo and used as is in the next reaction.

Step 2b: Crude 3-fluoro-5-formyl-4-hydroxybenzoyl chloride, (53 mg, 0.26 mmol) was dissolved in DCM (0.5 mL). 2-(4-(pyrrolidin-1-yl)phenyl)morpholine hydrochloride (Int 2, 161 mg, 0.24 mmol) and triethylamine (147 □L) were added. The reaction was stirred overnight at room temperature. The mixture was poured into water and extracted with DCM three times. The organic extracts were combined, washed with brine, dried over anhydrous sodium sulfate, and concentrated. The residue was purified by silica gel column chromatography 0-6% MeOH in DCM. The impure mixture was precipitated from DCM and Hexane, filtered and dried to give rac-3-fluoro-2-hydroxy-5-(2-(4-(pyrrolidin-1-yl)phenyl)morpholine-4-carbonyl)benzaldehyde (41.2 mg, 39% yield). ¹H NMR (500 MHz, CDCl3) δ 11.15 (s, 1H), 9.93 (s, 1H), 7.55 (s, 1H), 7.49-7.45 (m, 1H), 7.26 (bs, 2H), 6.53 (bs, 2H), 4.75-4.42 (bs, 2H), 4.09 (bs, 1H), 3.86-3.60 (m, 2H), 3.34 (m, 6H), 1.50 (m, 4H). LC-MS m/z [M+H]⁺ calc'd for C₂₂H₂₄FN₂O₄ 399, found 399.

### Example 42: rel-(S)-3-fluoro-2-hydroxy-5-(2-(4-(pyrrolidin-1-yl)phenyl)morpholine-4-carbonyl)benzaldehyde (Compound 148)

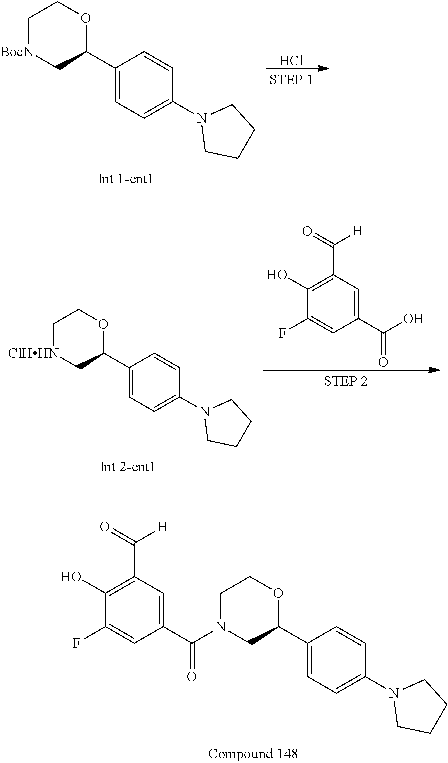

Step 1: Tert-Butyl 2-(4-(pyrrolidin-1-yl)phenyl)morpholine-4-carboxylate (1.2 g, 3.61 mmol) was purified by chiral prep-HPLC using a Superchiral R-AD column (Chiralway Biotech, 5 um, 2.1×25 cm) with an eluent of 50/50 MeOH/ACN at 15 ml/min, 35° C., and a wavelength of 220 nm. The first eluting peak was Int 1-ent1 and the second eluting peak was Int 1-ent2. (the two enantiomers were assigned randomly as rel-S and rel-R). In a 50 mL glass vial, Int1-ent1 (100 mg, 0.30 mmol, 1 eq.) was treated with 8 N HCl(gas)/dioxane (5 mL) for 2 h at rt. The reaction mixture was concentrated in vacuo and co-evaporated with DCM two times to give crude Int2-ent1 (85 mg, 0.30 mmol, quantitative yield), which was used for next reaction without further purification.

Step 2: In a 50 mL glass vial, HATU (148 mg, 0.39 mmol, 1.5 eq.) was added to a solution of 3-fluoro-5-formyl-4-hydroxybenzoic acid (48 mg, 0.26 mmol, 1 eq.), DIEA (101 mg, 0.78 mmol, 3 eq.), and Int2-ent1 (85 mg, 0.30 mmol, 1.15 eq.) in DMF (5 mL). The reaction was stirred overnight at rt. The solution was poured into water and extracted with ethyl acetate three times. The organic extracts were combined, washed with brine, dried over anhydrous sodium sulfate, and concentrated in vacuo. The residue was purified by silica gel column chromatography (PE/EA=10:1 to 3:1) and slurried in PE/DCM (10:1) for several times. Finally the cake was slurried in isopropyl ether for several times to give rel-(S)-3-fluoro-2-hydroxy-5-(2-(4-(pyrrolidin-1-yl)phenyl)morpholine-4-carbonyl)benzaldehyde (9.7 mg, 0.02 mmol, 9% yield) as a yellow solid. ¹H NMR (CDCl3, 400 MHz) δ: 11.16 (s, 1H), 9.94 (s, 1H), 7.55 (s, 1H), 7.47 (dd, J=10.4 Hz, 1.2 Hz, 1H), 7.21 (br, 2H), 6.61 (br, 2H), 4.75-4.27 (br, 2H), 4.09 (br, 1H), 3.73 (br, 2H), 3.30 (m, 6H), 2.02 (m, 4H). LC-MS m/z [M+H]⁺ calc'd for C₂₂H₂₄FN₂O₄ 399, found 399.

### Example 43: rel-(R)-3-fluoro-2-hydroxy-5-(2-(4-(pyrrolidin-1-yl)phenyl)morpholine-4-carbonyl)benzaldehyde (Compound 149)

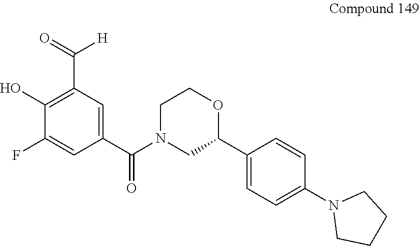 [SMILES: O=Cc1cc(C(=O)N2CCO[C@H](c3ccc(N4CCCC4)cc3)C2)cc(F)c1O]

The title compound was prepared using a method similar to that as described in Example 42 starting from Int 1-ent2 to give rel-(R)-3-fluoro-2-hydroxy-5-(2-(4-(pyrrolidin-1-yl)phenyl)morpholine-4-carbonyl)benzaldehyde (18.1 mg, 0.05 mmol, 17% yield) as a yellow solid. ¹H NMR (CDCl3, 400 MHz) δ: 11.17 (s, 1H), 9.94 (s, 1H), 7.56 (s, 1H), 7.47 (d, J=10.4 Hz, 1H), 7.26 (br, 2H), 6.71 (br, 2H), 4.75-4.27 (br, 2H), 4.09 (br, 1H), 3.68 (br, 2H), 3.34 (m, 6H), 2.07 (m, 4H). LC-MS m/z [M+H]⁺ calc'd for C₂₂H₂₄FN₂O₄ 399, found 399.

### Example 44: rac-3-fluoro-2-hydroxy-5-(2-(3-(pyrrolidin-1-yl)phenyl)morpholine-4-carbonyl)benzaldehyde (Compound 150)

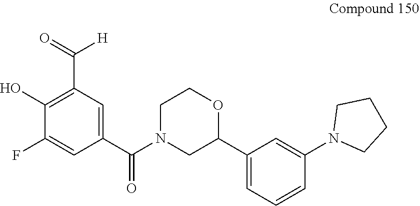 [SMILES: O=Cc1cc(C(=O)N2CCOC(c3cccc(N4CCCC4)c3)C2)cc(F)c1O]

The title compound was prepared using a method similar to that as described in Example 41 to give 32.3 mg of rac-3-fluoro-2-hydroxy-5-(2-(3-(pyrrolidin-1-yl)phenyl)morpholine-4-carbonyl)benzaldehyde in 28% yield for the final coupling step. ¹H NMR (500 MHz, CDCl3) δ 11.16 (s, 1H), 9.94 (dd, J=5.4, 1.7 Hz, 1H), 7.56 (d, J=7.5 Hz, 1H), 7.48 (dd, J=10.4, 2.0 Hz, 1H), 7.34 (d, J=17.2 Hz, 2H), 7.20 (t, J=7.9 Hz, 1H), 6.51 (d, J=8.5 Hz, 2H), 4.97-3.90 (m, 4H), 3.74 (s, 2H), 3.27 (d, J=6.2 Hz, 4H), 2.05-1.93 (m, 4H). LC-MS m/z [M+H]⁺ calc'd for C₂₂H₂₄FN₂O₄ 399, found 399.

### Example 45: rac-3-fluoro-2-hydroxy-5-((2S,3S)-3-methyl-2-(4-(pyrrolidin-1-yl)phenyl)morpholine-4-carbonyl)benzaldehyde (Compound 151)

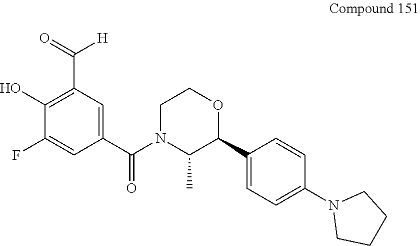 [SMILES: C[C@H]1[C@H](c2ccc(N3CCCC3)cc2)OCCN1C(=O)c1cc(F)c(O)c(C=O)c1]

The title compound was prepared using a method similar to that as described in Example 41 to give 10.4 mg of rac-3-fluoro-2-hydroxy-5-((2S,3S)-3-methyl-2-(4-(pyrrolidin-1-yl)phenyl)morpholine-4-carbonyl)benzaldehyde in 11% yield for the final coupling step. ¹H NMR (500 MHz, CDCl3) δ 11.12 (d, J=3.7 Hz, 1H), 9.90 (dd, J=2.6, 1.7 Hz, 1H), 7.45-7.29 (m, 4H), 7.24 (d, J=2.1 Hz, 1H), 6.58-6.53 (m, 1H), 4.97 (d, J=7.3 Hz, 1H), 4.85-4.53 (m, 1H), 3.81-3.39 (m, 4H), 3.36-3.02 (m, 4H), 2.20-1.83 (m, 4H), 1.55 (s, 3H). LC-MS m/z [M+H]⁺ calc'd for C₂₃H₂₆FN₂O₄, 413, Found 413.

### Example 46: rac-3-fluoro-2-hydroxy-5-(2-(4-methoxyphenyl)thiomorpholine-4-carbonyl)benzaldehyde (Compound 152)

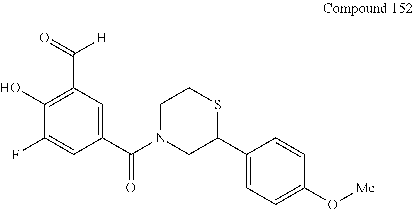 [SMILES: COc1ccc(C2CN(C(=O)c3cc(F)c(O)c(C=O)c3)CCS2)cc1]

The title compound was prepared using a method similar to that as described in Example 41 to give 27.2 mg of rac-3-fluoro-2-hydroxy-5-(2-(4-methoxyphenyl)thiomorpholine-4-carbonyl)benzaldehyde in 26% yield for the final coupling step. ¹H NMR (500 MHz, CDCl3) δ 11.13 (s, 1H), 9.92 (s, 1H), 7.48 (d, J=13.4 Hz, 1H), 7.45-7.37 (m, 1H), 7.33-7.16 (m, 2H), 6.93-6.76 (m, 2H), 4.73 (bs, 1H), 3.93 (bs, 1H), 3.79 (s, 4H), 3.35 (bs, 2H), 2.97 (bs, 1H), 2.67 (bs, 1H). LC-MS m/z [M+H]⁺ calc'd for C₁₉H₁₉FNO₄S 376, found 376.

### Example 47: (S)-3-fluoro-2-hydroxy-5-(2-(4-(pyrrolidin-1-yl)phenyl)thiomorpholine-4-carbonyl)benzaldehyde (Compound 153)

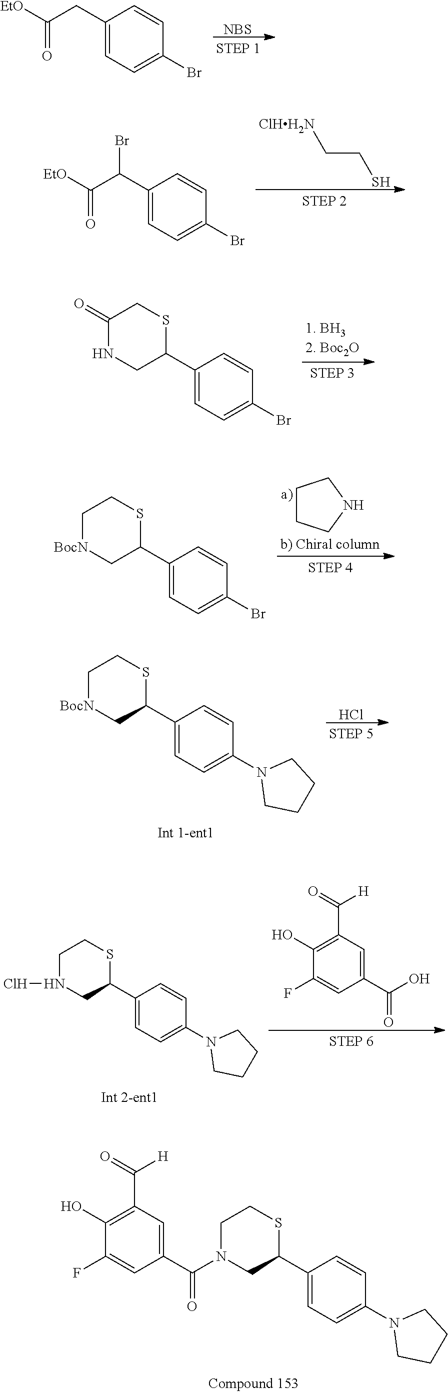

Step 1: NBS (8.0 g, 45.27 mmol, 1.1 eq.) and AIBN (675 mg, 4.12 mmol, 0.1 eq.) were added to a solution of ethyl 2-(4-bromophenyl)acetate (10 g, 41.15 mmol, 1 eq.) in CCl₄ (50 mL) at 0° C. under nitrogen protection. The reaction was stirred heated at 80° C. for 4 h. The mixture was poured into ice water and extracted with DCM three times. The organic extracts were combined, washed with water, dried over anhydrous sodium sulfate, and concentrated to give crude ethyl 2-bromo-2-(4-bromophenyl)acetate (14.9 g, 41.15 mmol, quantitative yield) as an oil.

Step 2: A mixture of 2-aminoethanethiol hydrochloride (5.26 g, 46.34 mmol, 1.1 eq.) and K₂CO₃ (12.8 g, 92.75 mmol, 2.3 eq.) in ethanol (50 mL) was stirred at 30° C. for 15 min. solution of tert-butyl ethyl 2-bromo-2-(4-bromophenyl)acetate (14.9 g, 41.15 mmol, 1 eq.) was added. The reaction was stirred overnight at 30° C. Ethanol was removed in vacuo. The residue was diluted with water and extracted with ethyl acetate three times. The organic extracts were combined, washed with brine, dried over anhydrous sodium sulfate, and concentrated. The residue was purified by silica gel column chromatography (PE/EA=30:1 to 10:1) to give 6-(4-bromophenyl)thiomorpholin-3-one (6.5 g, 23.81 mmol, 58% yield). LC-MS m/z [M+H]⁺ calc'd for C₁₀H₁₁BrNOS 273, found 273.7.

Step 3a: BH₃·Me₂S (22 mL, 1 M in THF, 21.98 mmol, 3 eq.) was added to a solution of 6-(4-bromophenyl)thiomorpholin-3-one (2 g, 7.33 mmol, 1 eq.) in THF (30 mL) at 0° C. The reaction was heated at 75° C. for 2 h. The system was cooled to rt, quenched with water, and extracted with ethyl acetate three times. The organic extracts were combined, washed with brine, dried over anhydrous sodium sulfate, and concentrated. The residue was heated in 4N HCl/THF (12 mL/12 mL) overnight at 50° C. The system was cooled to rt, neutralized to pH=8 with sodium carbonate, and extracted with ethyl acetate three times. The organic extracts were combined, washed with brine, dried over anhydrous sodium sulfate, and concentrated to give amine (1.7 g, 6.56 mmol, 90% yield). LC-MS m/z [M+H]⁺ calc'd for C₁₀H₁₃BrNS 359, found 359.7.

Step 3b: The amine ((1.7 g, 6.56 mmol, 1 eq.) was dissolved in THF/water (10 mL/10 mL). Sodium bicarbonate (1.38 g, 16.40 mmol, 2.5 eq.) and Boc2O (1.57 g, 7.20 mmol, 1.1 eq.) were added. The reaction was stirred overnight at rt. The solution was extracted with ethyl acetate three times. The organic extracts were combined, washed with brine, dried over anhydrous sodium sulfate, and concentrated to give crude tert-butyl 2-(4-bromophenyl)thiomorpholine-4-carboxylate (2.61 g, 7.27 mmol, quantitative yield), which was used for next reaction without further purification.

Step 4a: A mixture of 2-(4-bromophenyl)thiomorpholine-4-carboxylate (2.5 g, 6.96 mmol, 1 eq.), pyrrolidine (593 mg, 8.36 mmol, 1.2 eq.), tBuONa (1.47 g, 15.31 mmol, 2.2 eq.), BINAP (430 mg, 0.70 mmol, 0.1 eq.), and Pd₂(dba)₃ (320 mg, 0.70 mmol, 0.1 eq.) in toluene (30 mL) was heated at 85° C. for 5 h. The reaction mixture was cooled to rt, poured into water, and extracted with ethyl acetate. The organic phase was washed with brine, dried over anhydrous sodium sulfate, and concentrated. The residue was purified by silica gel column chromatography (PE/EA=200:1 to 50:1) to give tert-butyl 2-(4-(pyrrolidin-1-yl)phenyl)thiomorpholine-4-carboxylate (1.1 g, 3.16 mmol, 45% yield). LC-MS m/z [M+H]⁺ calc'd for C₁₉H₂₈N₂O₂S 349, found 349.

Step 4b: Tert-Butyl 2-(4-(pyrrolidin-1-yl)phenyl)thiomorpholine-4-carboxylate (1.1 g, 3.16 mmol, 1 eq.) was purified by chiral prep-HPLC using a Superchiral R-AD column (Chiralway Biotech, 5 um, 2.1×25 cm) with an eluent of 50/80 MeOH/ACN at 15 ml/min, 35° C., and a wavelength of 220 nm. The first eluting peak was Int 1-ent 1, (412 mg, 1.18 mmol) and the second eluting peak was Int 1-ent 2, (403 mg, 1.16 mmol), The two enantiomers were assigned randomly as rel-(S) and rel-(R).

Step 5: In a 50 mL glass vial, Int 1-ent 1 (100 mg, 0.29 mmol, 1 eq.) was treated with 8 N HCl(gas)/dioxane (5 mL) for 2 h at rt. The reaction mixture was concentrated in vacuo and co-evaporated with DCM two times to give crude Int 2-ent 1 (87 mg, 0.29 mmol, quantitative yield), which was used for next reaction without further purification.

Step 6: In a 50 mL glass vial, HATU (120 mg, 0.32 mmol, 1.2 eq.) was added to a solution of 3-fluoro-5-formyl-4-hydroxybenzoic acid (48 mg, 0.26 mmol, 1 eq.), TEA (79 mg, 0.78 mmol, 3 eq.), and Int 2-ent 1 (87 mg, 0.29 mmol, 1.1 eq.) in DCM (5 mL). The reaction was stirred overnight at rt. The solution was poured into water and pH was adjusted to 3-4. The mixture was extracted with ethyl acetate three times. The organic extracts were combined and concentrated in vacuo. The residue was purified by silica gel column chromatography (PE/EA/DCM=15:1:1 to 4:1:1) and prep-TLC to give the title compound, rel-(S)-3-fluoro-2-hydroxy-5-(2-(4-(pyrrolidin-1-yl)phenyl)thiomorpholine-4-carbonyl)benzaldehyde, (50 mg, 0.12 mmol, 46% yield) as a yellow solid. ¹H NMR (DMSO-d6, 400 MHz) δ: 11.37 (s, 1H), 10.28 (s, 1H), 7.64 (d, J=10.8 Hz, 1H), 7.55 (s, 1H), 7.12 (br, 2H), 6.47 (br, 2H), 4.70 (br, 1H), 3.99 (m, 2H), 3.38 (m, 1H), 3.18 (m, 4H), 2.92 (m, 2H), 2.65 (m, 1H), 1.93 (m, 4H). LC-MS m/z [M+H]⁺ calc'd for C₂₂H₂₄FN₂O₃S 415, found 415.

### Example 48: rel-(R)-3-fluoro-2-hydroxy-5-(2-(4-(pyrrolidin-1-yl)phenyl)thiomorpholine-4-carbonyl)benzaldehyde (Compound 154)

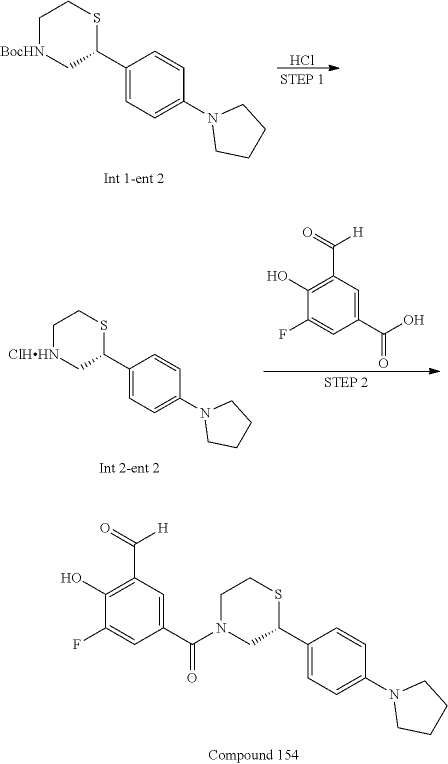

Step 1: In a 50 mL glass vial, Int1-ent2 (140 mg, 0.40 mmol, 1 eq.) was treated with 8 N HCl(gas)/dioxane (5 mL) for 2 h at rt. The reaction mixture was concentrated in vacuo and co-evaporated with DCM two times to give crude Int 2-ent 2 (123 mg, 0.40 mmol, quantitative yield), which was used for next reaction without further purification.

Step 2: In a 50 mL glass vial, HATU (166 mg, 0.44 mmol, 1.2 eq.) was added to a solution of 3-fluoro-5-formyl-4-hydroxybenzoic acid (67 mg, 0.36 mmol, 1 eq.), TEA (110 mg, 1.09 mmol, 3 eq.), and Int 2-ent 2 (123 mg, 0.40 mmol, 1.1 eq.) in DCM (5 mL). The reaction was stirred overnight at rt. The solution was poured into water and pH was adjusted to 3-4. The mixture was extracted with ethyl acetate three times. The organic extracts were combined and concentrated in vacuo. The residue was purified by silica gel column chromatography (PE/EA/DCM=15:1:1 to 4:1:1) and slurried in PE/DCM (10:1, 5 mL) to give the title compound, rel-(R)-3-fluoro-2-hydroxy-5-(2-(4-(pyrrolidin-1-yl)phenyl)thiomorpholine-4-carbonyl)benzaldehyde, (70 mg, 0.17 mmol, 47% yield) as a yellow solid. ¹H NMR (DMSO-d6, 400 MHz) δ: 11.37 (s, 1H), 10.28 (s, 1H), 7.64 (d, J=10.8 Hz, 1H), 7.55 (s, 1H), 7.14 (br, 2H), 6.47 (br, 2H), 4.70 (br, 1H), 3.99 (m, 2H), 3.38 (m, 1H), 3.18 (m, 4H), 2.92 (m, 2H), 2.68 (m, 1H), 1.93 (m, 4H).). LC-MS m/z [M+H]⁺ calc'd for C₂₂H₂₄FN₂O₃S 415, found 415.

### Example 49: rac-3-fluoro-2-hydroxy-5-(2-(4-(pyrrolidin-1-yl)phenyl)pyrrolidine-1-carbonyl)benzaldehyde (Compound 155)

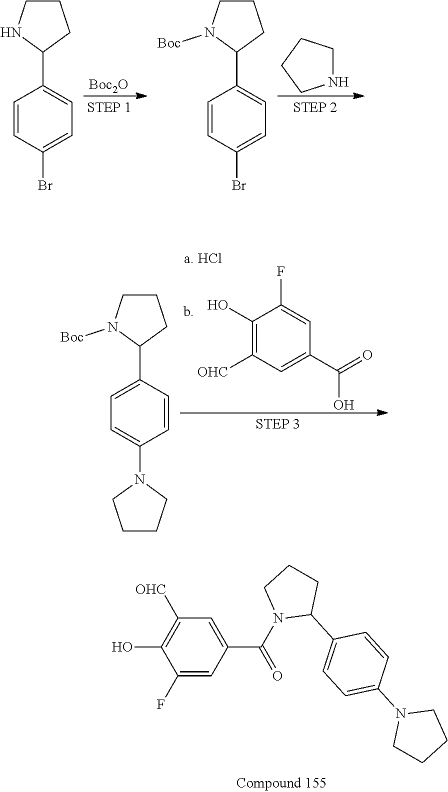

Step 1: 2-(4-Bromophenyl)pyrrolidine (400 mg, 1.77 mmol, 1 eq.) was dissolved in DCM (5 mL). TEA (537 mg, 5.31 mmol, 3 eq.) and Boc₂O (386 mg, 1.77 mmol, 1 eq.) were added. The reaction was stirred for 2 h at rt. The solution was extracted with DCM three times. The organic extracts were combined, washed with brine, dried over anhydrous sodium sulfate, and concentrated to give crude tert-butyl 2-(4-bromophenyl)pyrrolidine-1-carboxylate (790 mg, 1.77 mmol, quantitative yield), which was used for next reaction without further purification.

Step 2: A mixture of tert-butyl 2-(4-bromophenyl)pyrrolidine-1-carboxylate (350 mg, 1.07 mmol, 1 eq.), pyrrolidine (305 mg, 4.29 mmol, 4 eq.), Cs₂CO₃ (700 mg, 2.15 mmol, 2.0 eq.), X-Phos (63 mg, 0.11 mmol, 0.1 eq.), and Pd₂(dba)₃ (98 mg, 0.11 mmol, 0.1 eq.) in toluene (5 mL) was heated overnight at 95° C. The reaction mixture was cooled to rt, poured into water, and extracted with ethyl acetate. The organic phase was washed with brine, dried over anhydrous sodium sulfate, and concentrated. The residue was purified by silica gel column chromatography (PE/EA=200:1 to 100:1) to give tert-butyl 2-(4-(pyrrolidin-1-yl)phenyl)pyrrolidine-1-carboxylate (173 mg, 0.55 mmol, 51.1% yield). LC-MS m/z [M+H]⁺ calc'd for C₁₉H₂₉N₂O₂. 317, found 317

Step 3a: In a 50 mL glass vial, tert-butyl 2-(4-(pyrrolidin-1-yl)phenyl)pyrrolidine-1-carboxylate (173 mg, 0.55 mmol, 1 eq.) was treated with 8 N HCl(gas)/dioxane (5 mL) for 2 h at rt. The reaction mixture was concentrated in vacuo and co-evaporated with DCM two times to give crude 2-(4-(pyrrolidin-1-yl)phenyl)pyrrolidine hydrochloride (147 mg, 0.55 mmol, quantitative yield), which was used for next reaction without further purification.

Step 3b: In a 50 mL glass vial, HATU (263 mg, 0.69 mmol, 1.2 eq.) was added to a solution of 3-fluoro-5-formyl-4-hydroxybenzoic acid (106 mg, 0.58 mmol, 1.1 eq.), DIEA (223 mg, 1.73 mmol, 3 eq.), and 2-(4-(pyrrolidin-1-yl)phenyl)pyrrolidine hydrochloride (147 mg, 0.55 mmol, 1 eq.) in DMF (6 mL). The reaction was stirred for 3 h at rt. The solution was poured into water and extracted with ethyl acetate three times. The organic extracts were combined, washed with brine, dried over anhydrous sodium sulfate, and concentrated in vacuo. The residue was purified by silica gel column chromatography (PE/EA=10:1 to 5:1) and prep-TLC to give 3-fluoro-2-hydroxy-5-(2-(4-(pyrrolidin-1-yl)phenyl)pyrrolidine-1-carbonyl)benzaldehyde (22.6 mg, 0.06 mmol, 10.8% yield) as a yellow solid. ¹H NMR (CDCl₃, 400 MHz) δ: 11.19+11.00 (br, 1H), 9.96+9.52 (br, 1H), 7.72+7.62 (m, 1H), 7.52-7.25 (m, 3H), 7.01-6.90 (m, 1H), 6.55 (br, 1H), 5.24+4.78 (m, 1H), 3.95-3.71 (m, 2H), 3.29 (br, 4H), 2.41-1.83 (m, 8H). LC-MS m/z [M+H]⁺ calc'd for C₂₂H₂₃FN₂O₃ 383, found 383.

### Example 50: rel-(R)-3-fluoro-2-hydroxy-5-(3-(4-(pyrrolidin-1-yl)phenyl)pyrrolidine-1-carbonyl)benzaldehyde (Compound 156)

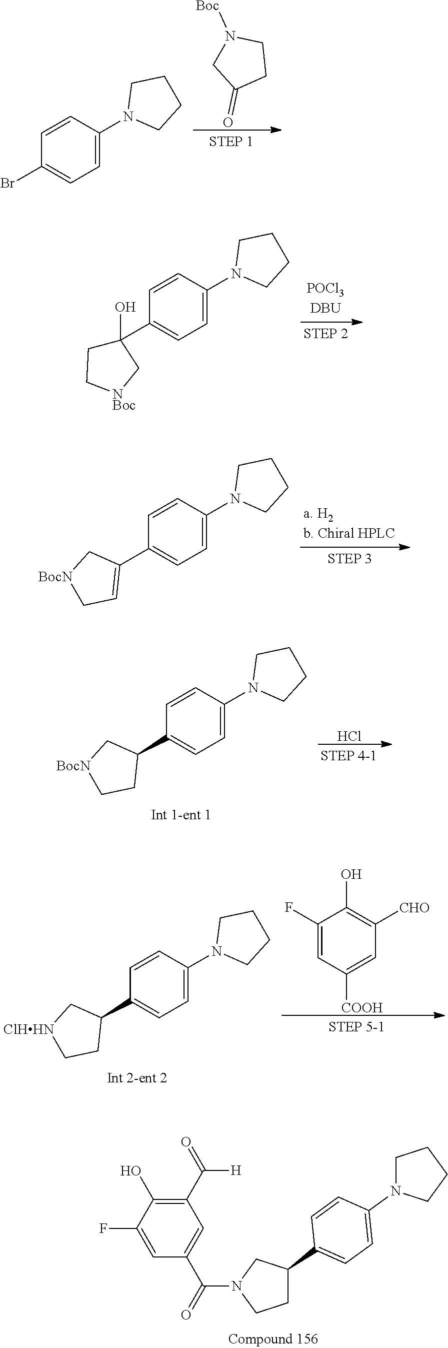

Step 1: BuLi (5.7 mL, 2.5 M in THF/hexane, 14.22 mmol, 1.6 eq.) was added to a solution of 1-(4-bromophenyl)pyrrolidine (2 g, 8.89 mmol, 1 eq.) in THF (20 mL) at −78° C. under nitrogen protection. The reaction was stirred for 30 min at −78° C. and a solution of tert-butyl 3-oxopyrrolidine-1-carboxylate (2.47 g, 13.33 mmol, 1.5 eq.) in THF (5 mL) was added dropwise. The cooling system was removed and the reaction was stirred for another 40 min. The mixture was poured into sat. NH₄Cl solution and extracted with ethyl acetate three times. The organic extracts were combined, washed with brine, dried over anhydrous sodium sulfate, and concentrated. The residue was slurried in PE/DCM (10:1, 10 mL) to give tert-butyl 3-hydroxy-3-(4-(pyrrolidin-1-yl)phenyl)pyrrolidine-1-carboxylate (0.85 g, 2.56 mmol, 29% yield) as a solid.). LC-MS m/z [M+H]⁺ calc'd for C₁₉H₂₉N₂O₃ 333, Found 333.

Step 2: POCl₃ (1.71 g, 11.20 mmol, 3 eq.) was added dropwise to a solution of tert-butyl 3-hydroxy-3-(4-(pyrrolidin-1-yl)phenyl)pyrrolidine-1-carboxylate (1.24 g, 3.73 mmol, 1 eq.) and DBU (1.70 g, 11.20 mmol, 5 eq.) in pyridine (5 mL) at 0° C. The reaction was heated at 80° C. for 2 h. The solution was cooled to rt, poured into water, and extracted with ethyl acetate three times. The organic extracts were combined, washed with brine, dried over anhydrous sodium sulfate, and concentrated. The residue was purified by silica gel column chromatography (PE/EA=20:1 to 5:1) to give tert-butyl 3-(4-(pyrrolidin-1-yl)phenyl)-2,5-dihydro-1H-pyrrole-1-carboxylate (1.01 g, 3.22 mmol, 86% yield). LC-MS m/z [M+H]⁺ calc'd for C₁₉H₂₇N₂O₂315, found 315

Step 3a: A solution of tert-butyl 3-(4-(pyrrolidin-1-yl)phenyl)-2,5-dihydro-1H-pyrrole-1-carboxylate (1.01 g, 3.22 mmol, 1 eq.) in MeOH (10 mL) was hydrogenated with Pd/C (200 mg) for 2 h at rt. Pd/C was filtered off and the filtration was concentrated in vacuo to give tert-butyl 3-(4-(pyrrolidin-1-yl)phenyl)pyrrolidine-1-carboxylate (1.02 g, 3.22 mmol, quantitative yield). LC-MS m/z [M+H]⁺ calc'd for C₁₉H₂₈N₂O₂ 317, Found 317.

Step 3b: Part of the crude was separated by chiral HPLC using a Superchiral R-AD column (Chiralway Biotech, 5 um, 2.1×25 cm) with an eluent of 50/50/0.05 MeOH/ACN/diethylamine at 15 ml/min, 35° C., and a wavelength of 220 nm. Peak 1 corresponds to Int 1-ent 1, rel-(R)-tert-butyl 3-(4-(pyrrolidin-1-yl)phenyl)pyrrolidine-1-carboxylate (134 mg, peak 1) and peak 2 corresponds to Int 1-ent 2, rel-(S)-tert-butyl 3-(4-(pyrrolidin-1-yl)phenyl)pyrrolidine-1-carboxylate (145 mg, peak 2). The rel-(R) and rel-(S)-enantiomers were assigned randomly.

Step 4-1: In a 50 mL glass vial, Int 1-ent 1 (134 mg, 0.42 mmol, 1 eq.) was treated with 8 N HCl(gas)/dioxane (5 mL) for 2 h at rt. The reaction mixture was concentrated in vacuo and co-evaporated with DCM two times to give crude Int 2-ent 1 (145 mg, 0.42 mmol, quantitative yield), which was used for next reaction without further purification.

Step 5-1: In a 50 mL glass vial, EDCI (96 mg, 0.50 mmol, 1.2 eq.) was added to a solution of 3-fluoro-5-formyl-4-hydroxybenzoic acid (93 mg, 0.50 mmol, 1.2 eq.), TEA (212 mg, 2.10 mmol, 5 eq.), and Int 2-ent 1 (145 mg, 0.42 mmol, 1 eq.) in DCM (5 mL). The reaction was stirred overnight at rt. The solution was poured into water and extracted with DCM three times. The organic extracts were combined and concentrated in vacuo. The residue was purified by silica gel column chromatography (DCM/acetone=50:1 to 15:1) to give rel-(R)-3-fluoro-2-hydroxy-5-(3-(4-(pyrrolidin-1-yl)phenyl)pyrrolidine-1-carbonyl)benzaldehyde (15 mg, 0.04 mmol, 9% yield) as a yellow solid. Pos. LC-MS: 383.0 (M+H)⁺, C₂₂H₂₃FN₂O₃. ¹H NMR (CDCl₃, 400 MHz) δ: 11.17-11.15 (m, 1H), 9.96-9.92 (m, 1H), 7.72-7.67 (m, 1H), 7.63-7.59 (m, 1H), 7.16 (m, 1H), 7.09 (m, 1H), 6.60 (br, 2H), 4.06-3.86 (m, 2H), 3.78-3.62 (m, 2H), 3.49-3.38 (m, 2H), 3.29 (m, 4H), 2.35 (m, 1H), 2.26-2.10 (m, 2H), 2.02 (m, 4H).

### Example 51: rel-(S)-3-fluoro-2-hydroxy-5-(3-(4-(pyrrolidin-1-yl)phenyl)pyrrolidine-1-carbonyl)benzaldehyde (Compound 157)

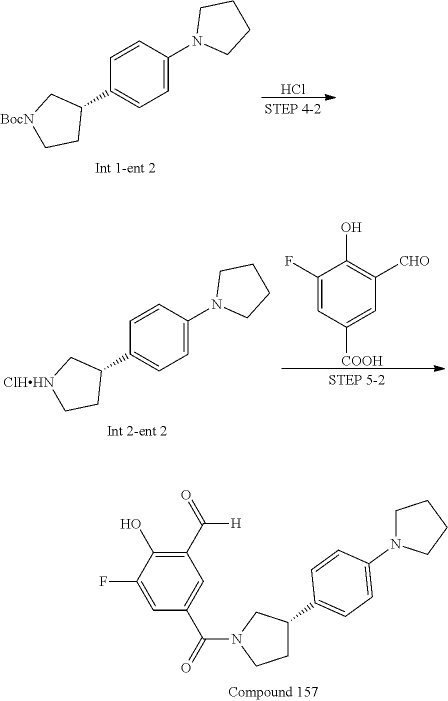

Step 4-2: In a 50 mL glass vial, Int 1-ent 2 (145 mg, 0.46 mmol, 1 eq.) was treated with 8 N HCl(gas)/dioxane (5 mL) for 2 h at rt. The reaction mixture was concentrated in vacuo and co-evaporated with DCM two times to give crude Int 2-ent 2 (153 mg, 0.46 mmol, quantitative yield), which was used for next reaction without further purification.

Step 5-2: In a 50 mL glass vial, EDCI (105 mg, 0.55 mmol, 1.2 eq.) was added to a solution of 3-fluoro-5-formyl-4-hydroxybenzoic acid (102 mg, 0.55 mmol, 1.2 eq.), TEA (212 mg, 2.10 mmol, 5 eq.), and Int 2-ent 2 (153 mg, 0.46 mmol, 1 eq.) in DCM (5 mL). The reaction was stirred overnight at rt. The solution was poured into water and extracted with DCM three times. The organic extracts were combined and concentrated in vacuo. The residue was purified by silica gel column chromatography (DCM/acetone=50:1 to 15:1) to give rel-(S)-3-fluoro-2-hydroxy-5-(3-(4-(pyrrolidin-1-yl)phenyl)pyrrolidine-1-carbonyl)benzaldehyde (17 mg, 0.04 mmol, 10% yield) as a yellow solid. ¹H NMR (CDCl₃, 400 MHz) δ: 11.17-11.15 (m, 1H), 9.96-9.92 (m, 1H), 7.72-7.63 (m, 1H), 7.57 (s, 1H), 7.62-7.59 (m, 1H), 7.18 (m, 1H), 7.13 (m, 1H), 6.67 (br, 2H), 4.08-3.82 (m, 2H), 3.82-3.59 (m, 2H), 3.53-3.38 (m, 2H), 3.32 (m, 4H), 2.33 (m, 1H), 2.23-2.10 (m, 2H), 2.05 (m, 4H). LC-MS m/z [M+H]⁺ calc'd for C₂₂H₂₃FN₂O₃ 383, found 383.

### Example 52: 3-fluoro-5-formyl-4-hydroxy-N-methyl-N-(3-phenylbicyclo[1.1.1]pentan-1-yl)benzamide (Compound 158)

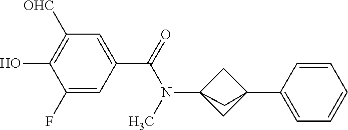 [SMILES: CN(C(=O)c1cc(F)c(O)c(C=O)c1)C12CC(c3ccccc3)(C1)C2]

The title compound was synthesized in a similar manner to Example 41 starting from N-methyl-3-phenylbicyclo[1.1.1]pentan-1-amine hydrobromide to give 3-fluoro-5-formyl-4-hydroxy-N-methyl-N-(3-phenylbicyclo[1.1.1]pentan-1-yl)benzamide (9 mg, 9% yield). LC-MS m/z [M+H]⁺ calc'd for C₂₀H₁₉FNO₃ 340, found 340.

### Example 53: 3-fluoro-5-formyl-4-hydroxybenzyl (3-(pyrrolidin-1-yl)phenyl)carbamate (Compound 159)

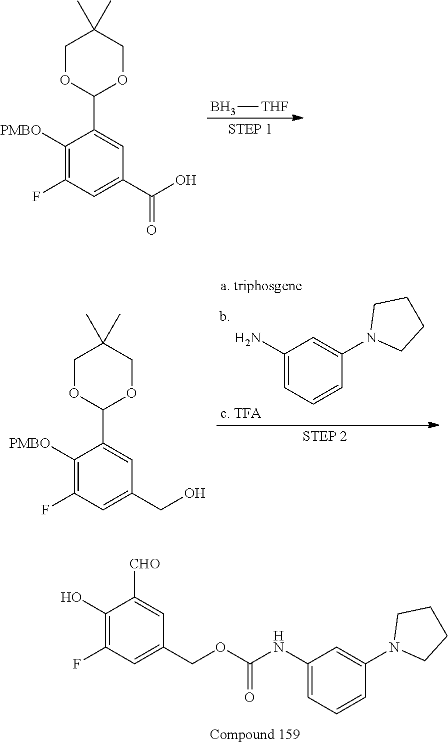

Step 1: A solution of 4-(benzyloxy)-3-(5,5-dimethyl-1,3-dioxan-2-yl)-5-fluorobenzoic acid (1.3 g, 3.33 mmol, 1 eq.) and BH₃·THF (10 mL, 10.0 mmol, 1 M in THF, 3 eq.) in THF (20 mL) was heated at 75° C. for 2 h. The solution was cooled to rt and quenched with ice-water. THF was removed in vacuo and the mixture was extracted with ethyl acetate three times. The organic extracts were combined, dried over anhydrous sodium sulfate, and concentrated. The residue was purified by silica gel column chromatography (PE/EA=20:1 to 5:1) to give (3-(5,5-dimethyl-1,3-dioxan-2-yl)-5-fluoro-4-(4-methoxybenzyloxy)phenyl)methanol (890 mg, 2.37 mmol, 71% yield). LC-MS m/z [M+H]⁺ calc'd for: C₂₁H₂₆FO₅ 399, found 399.

Step 2a: A solution of triphosgene (526 mg, 1.77 mmol, 0.75 eq.) was added dropwise to a solution of (3-(5,5-dimethyl-1,3-dioxan-2-yl)-5-fluoro-4-(4-methoxybenzyloxy)phenyl)methanol (890 mg, 2.37 mmol, 1 eq.) in THF (10 mL) at 0° C. The reaction was stirred for 1 h at 0° C.

Step 2b: A solution of 3-(pyrrolidin-1-yl)aniline (364 mg, 2.25 mmol, 0.95 eq.) in THF (2 mL) was added followed by TEA (720 mg, 7.11 mmol, 3 eq.). The reaction was stirred overnight at rt. The solution was poured into ice-water and extracted with ethyl acetate three times. The organic phase was dried over anhydrous sodium sulfate and concentrated. The residue was purified by silica gel column chromatography (PE/EA=20:1 to 5:1) to give 3-(5,5-dimethyl-1,3-dioxan-2-yl)-5-fluoro-4-(4-methoxybenzyloxy)benzyl 3-(pyrrolidin-1-yl)phenylcarbamate (434 mg, 0.77 mmol, 32% yield). LC-MS m/z [M+H]⁺ calc'd for: C₃₂H₃₈FN₂O₆ 565, found 565.

Step 2c: 3-(5,5-Dimethyl-1,3-dioxan-2-yl)-5-fluoro-4-(4-methoxybenzyloxy)benzyl 3-(pyrrolidin-1-yl)phenylcarbamate (150 mg, 0.27 mmol, 1 eq.) was added to a mixture of TFA (6 mL) in DCM/water (6 mL/3 mL). The reaction was stirred for 30 min at rt. The mixture was poured into ice-cold sodium bicarbonate slowly and extracted with DCM three times. The organic extracts were combined, washed with water, dried over anhydrous sodium sulfate, and concentrated in vacuo. The residue dissolved in PE/DCM (10:1) and concentrated. During concentration, a precipitate was formed. The precipitate was collected and re-treated with PE/DCM to give the title compound, 3-fluoro-5-formyl-4-hydroxybenzyl 3-(pyrrolidin-1-yl)phenylcarbamate, (27 mg, 0.08 mmol, 28% yield) as a yellow solid. ¹H NMR (DMSO-d6, 400 MHz) δ: 11.05 (s, 1H), 10.30 (s, 1H), 9.54 (br, 1H), 7.63-7.56 (m, 2H), 7.02 (t, J=8.0 Hz, 1H), 6.74 (s, 1H), 6.69 (d, J=7.6 Hz, 1H), 6.18 (dd, J=8.0 Hz, 2.0 Hz, 1H), 5.07 (s, 2H), 3.16 (m, 4H), 1.93 (m, 4H). LC-MS m/z [M+H]⁺ calc'd for: C₁₉H₂₀FN₂O₄ 359, found 359.

### Example 54: N′-(3-bromophenyl)-3-fluoro-5-formyl-4-hydroxybenzohydrazide (Compound 160)

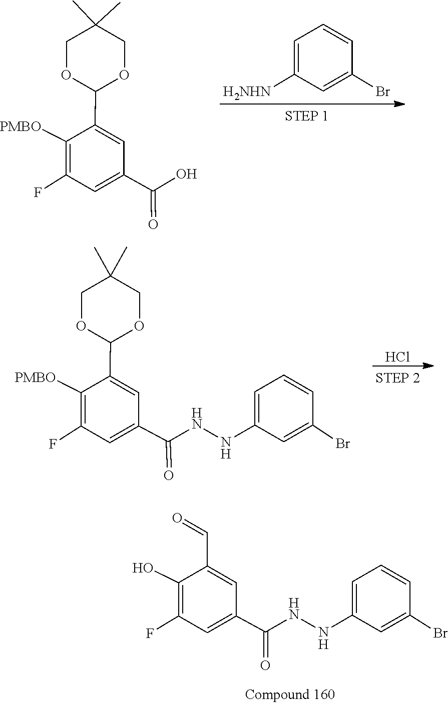

Step 1: In a 100 mL glass vial, a solution of 3-(5,5-dimethyl-1,3-dioxan-2-yl)-5-fluoro-4-(4-methoxybenzyloxy)benzoic acid (175 mg, 0.45 mmol, 1 eq.), (3-bromophenyl)hydrazine (88 mg, 0.47 mmol, 1.05 eq.), HATU (204 mg, 0.54 mmol, 1.2 eq.), and DIEA (174 mg, 1.35 mmol, 3 eq.) in DMF (5 mL) was stirred for 4 h at rt. The solution was poured into sat. NaHCO₃ and extracted with ethyl acetate three times. The organic extracts were combined, washed with brine, dried over anhydrous sodium sulfate, and concentrated. The residue was purified by silica gel column chromatography (PE/EA=20:1 to 5:1) and slurried in PE/DCM (10:1) to give N′-(3-bromophenyl)-3-(5,5-dimethyl-1,3-dioxan-2-yl)-5-fluoro-4-(4-methoxybenzyloxy)benzohydrazide (120 mg, 0.21 mmol, 47.7% yield).

Step 2: N′-(3-bromophenyl)-3-(5,5-dimethyl-1,3-dioxan-2-yl)-5-fluoro-4-(4-methoxybenzyloxy)benzohydrazide (110 mg, 0.20 mmol, 1 eq.) was dissolved in dioxane (2 mL) and 6 N HCl (gas)/dioxane solution (7 mL) was added. The reaction was stirred for 4 h at rt. The solvent was removed, and the residue was diluted with 10% sodium bicarbonate solution. The mixture was extracted with ethyl acetate three times. The organic extracts were combined, washed with brine, dried over anhydrous sodium sulfate, and concentrated. The residue was purified by prep-TLC to give N′-(3-bromophenyl)-3-fluoro-5-formyl-4-hydroxybenzohydrazide (130 mg, 0.37 mmol, 74% yield) as a brown solid. ¹H NMR (CDCl₃, 400 MHz) δ: 11.35 (s, 1H), 10.00 (s, 1H), 8.03 (br, 1H), 7.99 (s, 1H), 7.86 (d, J=10.8 Hz, 1H), 7.28 (m, 1H), 7.13-6.99 (m, 3H), 6.84 (d, J=8.0 Hz, 1H). Pos. LC-MS: 351.0 (M−H)⁻). LC-MS m/z [M−H]− calc'd for C₁₄H₉BrFN₂O₃ 351, found 351.

### Example 55: 3-fluoro-2-hydroxy-5-(4-(4-(pyrrolidin-1-yl)phenyl)piperidin-1-ylsulfonyl)benzaldehyde (Compound 161)

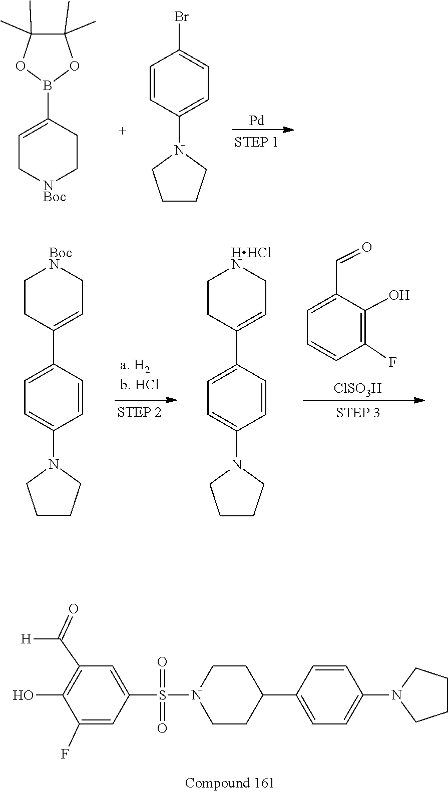

Step 1: A mixture of tert-butyl 4-(4,4,5,5-tetramethyl-1,3,2-dioxaborolan-2-yl)-5,6-dihydropyridine-1(2H)-carboxylate (7.52 g, 24.3 mmol, 1.1 eq.), 1-(4-bromophenyl)pyrrolidine (5 g, 22.1 mmol, 1 eq.), sodium carbonate (4.69 g, 44.2 mmol, 2 eq.), and PdCl₂(dppf) (1.62 g, 2.21 mmol, 0.1 eq.) in DMF/water (25 mL/10 mL) was heated at 80° C. for 2 h under N2 protection. The mixture was cooled to rt, diluted with ethyl acetate, and filtered with celite. The cake was washed with ethyl acetate. The filtrate and wash were combined, washed with water, dried over anhydrous sodium sulfate, and concentrated. The residue was purified by silica gel column (PE/EA=200:1 to 50:1) to give tert-butyl 4-(4-(pyrrolidin-1-yl)phenyl)-5,6-dihydropyridine-1(2H)-carboxylate (4.1 g, 12.5 mmol, 56.5% yield). LC-MS m/z [M+H]⁺ calc'd for: C₂₀H₂₉N₂O₂ 329, Found 329.

Step 2a: In a 100 mL glass vial, tert-butyl 4-(4-(pyrrolidin-1-yl)phenyl)-5,6-dihydropyridine-1(2H)-carboxylate (4.1 g, 12.5 mmol, 1 eq.) was hydrogenated in methanol (45 mL) with Pd/C (0.8 g) for 2 h. Pd/C was filtered off with celite and the filtrate was concentrated in vacuo to give intermediate (4.2 g, 12.5 mmol, quantitative yield) as a white solid.

Step 2b: The intermediate (4.2 g, 12.5 mmol, 1 eq.) was dissolved in DCM (10 mL) and 6N HCl(gas)/dioxane (12 mL) was added. The reaction was stirred for 2 h. The solvent was removed in vacuo and the residue was co-evaporated with DCM two times to give crude 4-(4-(pyrrolidin-1-yl)phenyl)piperidine hydrochloride (4.8 g, 12.5 mmol, quantitative yield), which was used for next reaction without further purification. LC-MS m/z [M+H]⁺ calc'd for: C₁₅H₂₄ClN₂ 231, found 231.

Step 3: In a 100 mL glass vial, ClSO₃H (8.3 g, 71.3 mmol, 10 eq.) was added dropwise to a solution of 3-fluoro-2-hydroxybenzaldehyde (1 g, 7.13 mmol, 1 eq.) in DCM (30 mL) at 0° C. The reaction was stirred at 0-5° C. for 7 h. The reaction mixture was poured into ice-water (50 mL) and extracted with DCM three times. The organic extracts were combined, washed with 6N H₂SO₄, dried over anhydrous sodium sulfate, and concentrated to give crude 3-fluoro-5-formyl-4-hydroxybenzene-1-sulfonyl chloride (1 g, 58.8% yield), which was used for next reaction without further purification. Neg. LC-MS: 219.26 (M-Cl+OH—H)⁻, LC-MS m/z [M−H]− calc'd for C₇H₄ClFO₄S.

In a 100 mL glass vial, a solution of 3-fluoro-5-formyl-4-hydroxybenzene-1-sulfonyl chloride (1 g, 4.19 mmol, 1 eq.), 4-(4-(pyrrolidin-1-yl)phenyl)piperidine hydrochloride (1.2 g, 4.50 mmol, 1.1 eq.), and TEA (4.23 g, 41.9 mmol, 10 eq.) in DCM (20 mL) was stirred for 5 h. The reaction mixture was poured into water, acidified to pH 3-4, and extracted with ethyl acetate three times. The organic extracts were combined, washed with brine, dried over anhydrous sodium sulfate, and concentrated. The residue was purified by silica gel column chromatography (PE/DCM=2:1 to 1:2) to give 3-fluoro-2-hydroxy-5-(4-(4-(pyrrolidin-1-yl)phenyl)piperidin-1-ylsulfonyl)benzaldehyde (90 mg, 0.21 mmol, 49.7% yield) as a white solid. ¹H NMR (DMSO-d6, 400 MHz) δ: 12.13 (br, 1H), 10.34 (s, 1H), 7.87 (dd, J=10.4 Hz, 2.4 Hz, 1H), 7.80 (d, J=1.6 Hz, 1H), 6.97 (d, J=8.8 Hz, 2H), 6.45 (d, J=8.4 Hz, 2H), 3.72 (m, 2H), 3.16 (m, 4H), 2.33 (m, 3H), 1.91 (m, 4H), 1.75 (m, 2H), 1.60 (m, 2H). LC-MS m/z [M+H]⁺ calc'd for C₂₂H₂₆FN₂O₄S 433, found 433.

### Example 56: 2-fluoro-6-formyl-4-(3-(4-(pyrrolidin-1-yl)phenyl)-1,2,4-thiadiazol-5-yl)phenyl tetrahydrofuran-3-yl carbonate (Compound 162)

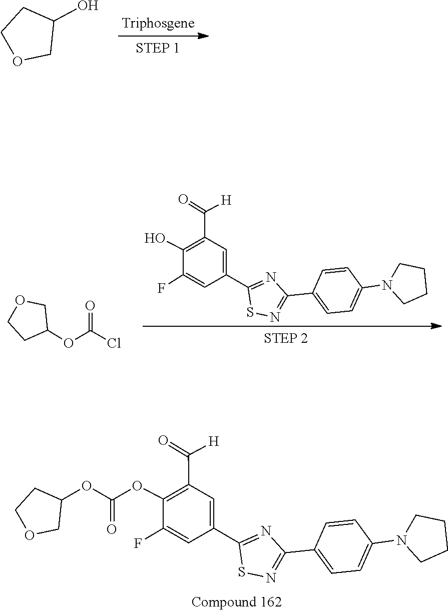 [SMILES: OC1CCOC1 ; O=C(Cl)OC1CCOC1 ; O=Cc1cc(-c2nc(-c3ccc(N4CCCC4)cc3)ns2)cc(F)c1O ; O=Cc1cc(-c2nc(-c3ccc(N4CCCC4)cc3)ns2)cc(F)c1OC(=O)OC1CCOC1]

Step 1: Na₂CO₃ (361 mg, 3.41 mmol, 1 eq.) and DMF (2.5 mg, 0.03 mmol, 0.01 eq.) were added to a solution of triphosgene (505 mg, 1.71 mmol, 0.5 eq.) in toluene (5 mL) at 0° C. The reaction was stirred for 30 min at 0° C. Then a solution of tetrahydrofuran-3-ol (300 mg, 3.41 mmol, 1 eq.) in toluene (2 mL) was added. The reaction was stirred overnight at rt. The solid was filtered off and the filtrate was concentrated in vacuo. The residue, tetrahydrofuran-3-yl carbonochloridate, (263 mg, 1.75 mmol, 51% yield) was used for next reaction without further purification.

Step 2: A solution of the residue in Step 1 (263 mg, 1.75 mmol, 4.0 eq.) in DCM (2 mL) was added to a solution of 3-fluoro-2-hydroxy-5-(3-(4-(pyrrolidin-1-yl)phenyl)-1,2,4-thiadiazol-5-yl)benzaldehyde (162 mg, 0.44 mmol, 1 eq.) in DCM (10 mL) and TEA (111 mg, 1.10 mmol, 2.5 eq.) at 0° C. The reaction was stirred heated at 40° C. for 1 h. The system was poured into cooled acidic (KHSO₄) water and extracted with DCM two times. The solution was diluted with PE and the resulting precipitate was filtered. The cake was slurried in PE/DCM (10:1) for 3 times to give 2-fluoro-6-formyl-4-(3-(4-(pyrrolidin-1-yl)phenyl)-1,2,4-thiadiazol-5-yl)phenyl tetrahydrofuran-3-yl carbonate (55 mg, 0.11 mmol, 26% yield) as a yellow solid. LC-MS m/z [M+H]⁺ calc'd for C₂₄H₂₃FN₃O₅S 484, found 484.

### Example 57: 3-fluoro-4-hydroxy-5-(((4-methylpiperazin-1-yl)imino)methyl)phenyl)(4-(4-(pyrrolidin-1-yl)phenyl)piperidin-1-yl)methanone (Compound 163)

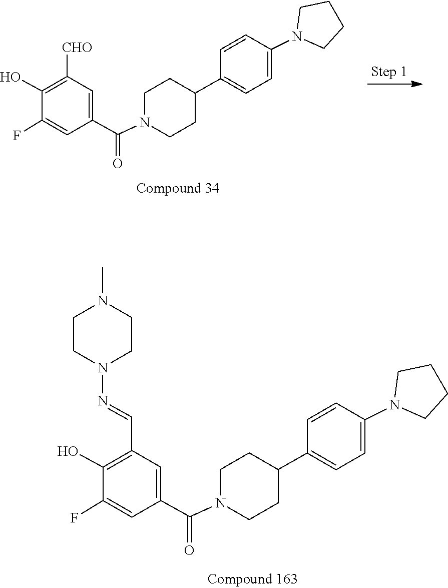 [SMILES: O=Cc1cc(C(=O)N2CCC(c3ccc(N4CCCC4)cc3)CC2)cc(F)c1O ; CN1CCN(/N=C/c2cc(C(=O)N3CCC(c4ccc(N5CCCC5)cc4)CC3)cc(F)c2O)CC1]

Step 1: (3-fluoro-2-hydroxy-5-(4-(4-(pyrrolidin-1-yl)phenyl)piperidine-1-carbonyl)benzaldehyde (Compound 34, 50 mg, 0.126 mmol) and 4-methylpiperazin-1-amine (45.5 □L, 0.38 mmol) were dissolved in ethanol (3.8 mL). The reaction was refluxed for 1 h. The resulting precipitate was filtered, washed with ethanol, and dried in vacuo to provide (3-fluoro-4-hydroxy-5-(((4-methylpiperazin-1-yl)imino)methyl)phenyl)(4-(4-(pyrrolidin-1-yl)phenyl)piperidin-1-yl)methanone (42.4 mg, 0.086 mmol) in 62% yield. ¹H NMR (DMSO-d6, 500 MHz 11.87 (s, 1H), 7.97 (s, 1H), 7.31 (d, J=1.9 Hz, 1H), 7.23 (dd, J=11.1, 2.0 Hz, 1H), 7.08-6.99 (m, 2H), 6.54-6.38 (m, 2H), 3.16 (dt, J=9.4, 5.6 Hz, 8H), 2.99 (m, 4H), 2.65 (m, 1H), 2.51 (d, J=5.5 Hz, 4H), 2.23 (s, 3H), 1.97-1.89 (m, 4H), 1.74 (m, 2H), 1.54 (m, 2H). LC-MS m/z [M+H]⁺ calc'd for C₂₈H₃₇FN₅O₂ 494; found, 494.

### Example 58: (3-fluoro-4-hydroxy-5-((morpholinoimino)methyl)phenyl)(4-(4-(pyrrolidin-1-yl)phenyl)piperidin-1-yl)methanone (Compound 164)

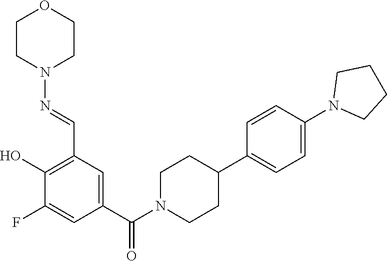 [SMILES: O=C(c1cc(F)c(O)c(/C=N/N2CCOCC2)c1)N1CCC(c2ccc(N3CCCC3)cc2)CC1]

The title compound was prepared from Compound 34 (33.5 mg, 0.084 mmol) and morpholin-4-amine (25 □L, 0.25 mmol) using a method similar to that as described in Example 57 to give (3-fluoro-4-hydroxy-5-((morpholinoimino)methyl)phenyl)(4-(4-(pyrrolidin-1-yl)phenyl)piperidin-1-yl)methanone (16.3 mg, 0.034 mmol, 40% yield). ¹H NMR (500 MHz, DMSO-d6) δ 11.76 (s, 1H), 8.03 (s, 1H), 7.33 (dd, J=1.9, 0.9 Hz, 1H), 7.25 (dd, J=11.1, 1.9 Hz, 1H), 7.10-7.00 (m, 2H), 6.52-6.42 (m, 2H), 3.85-3.73 (m, 4H), 3.23-3.11 (m, 8H), 3.00 (m, 4H), 2.72-2.56 (m, 1H), 1.95-1.89 (m, 4H), 1.74 (m, 2H), 1.54 (qd, J=12.6, 4.2 Hz, 2H). LC-MS m/z [M+H]⁺ calc'd for C₂₇H₃₄FN₄O₃ 481, found 481.

### Example 59: 2-fluoro-6-(((4-methylpiperazin-1-yl)imino)methyl)-4-(3-(6-(pyrrolidin-1-yl)pyridin-3-yl)-1,2,4-thiadiazol-5-yl)phenol (Compound 165)

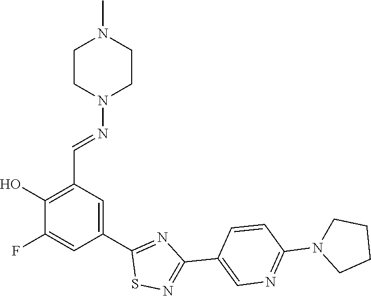 [SMILES: CN1CCN(/N=C/c2cc(-c3nc(-c4ccc(N5CCCC5)nc4)ns3)cc(F)c2O)CC1]

The title compound was prepared from Compound 130 (15.5 mg, 0.042 mmol) and 4-methylpiperazin-1-amine (15 □L, 0.125 mmol) using a method similar to that as described in Example 57 to give 2-fluoro-6-(((4-methylpiperazin-1-yl)imino)methyl)-4-(3-(6-(pyrrolidin-1-yl)pyridin-3-yl)-1,2,4-thiadiazol-5-yl)phenol (12.3 mg, 0.026 mmol, 63% yield). ¹H NMR (500 MHz, DMSO-d6) δ 12.25 (s, 1H), 8.99 (d, J=2.3 Hz, 1H), 8.27 (dd, J=8.9, 2.4 Hz, 1H), 8.07 (s, 1H), 8.01 (d, J=2.2 Hz, 1H), 7.86 (d, J=11.3 Hz, 1H), 6.59 (d, J=8.9 Hz, 1H), 3.48 (s, 4H), 3.19 (t, J=5.1 Hz, 4H), 2.53 (t, J=5.2 Hz, 4H), 2.25 (s, 3H), 2.00-1.93 (m, 4H). LC-MS m/z [M+H]⁺ calc'd for C₂₃H₂₇FN₇OS 468; found, 468.

### Example 60: 2-fluoro-6-((morpholinoimino)methyl)-4-(3-(4-(pyrrolidin-1-yl)phenyl)-1,2,4-thiadiazol-5-yl)phenol (Compound 166)

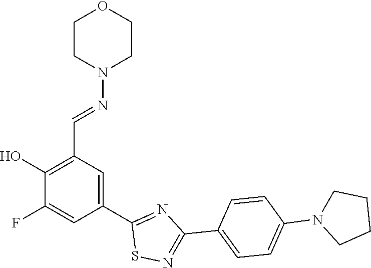 [SMILES: Oc1c(F)cc(-c2nc(-c3ccc(N4CCCC4)cc3)ns2)cc1/C=N/N1CCOCC1]

The title compound was prepared from 3-fluoro-2-hydroxy-5-(3-(4-(pyrrolidin-1-yl)phenyl)-1,2,4-thiadiazol-5-yl)benzaldehyde (28.9 mg, 0.078 mmol) and morpholin-4-amine (23 □L, 0.23 mmol) using a method similar to that as described in Example 57 to give 2-fluoro-6-((morpholinoimino)methyl)-4-(3-(4-(pyrrolidin-1-yl)phenyl)-1,2,4-thiadiazol-5-yl)phenol (30.3 mg, 0.067 mmol, 85% yield). ¹H NMR (500 MHz, DMSO-d6) δ 12.15 (s, 1H), 8.13 (s, 1H), 8.10 (d, J=8.5 Hz, 2H), 8.01 (d, J=2.1 Hz, 1H), 7.87 (dd, J=11.2, 2.2 Hz, 1H), 6.64 (d, J=8.5 Hz, 2H), 3.81 (t, J=4.8 Hz, 4H), 3.31 (s, 4H), 3.19 (t, J=4.9 Hz, 4H), 2.05-1.86 (m, 4H). LC-MS m/z [M+H]⁺ calc'd for C₂₃H₂₄FN₅O2_{S} 454, found 454.

### Example 61: 3-fluoro-5-formyl-4-hydroxy-N-(3-(4-(pyrrolidin-1-yl)phenyl)-1,2,4-thiadiazol-5-yl)benzamide (Compound 167)

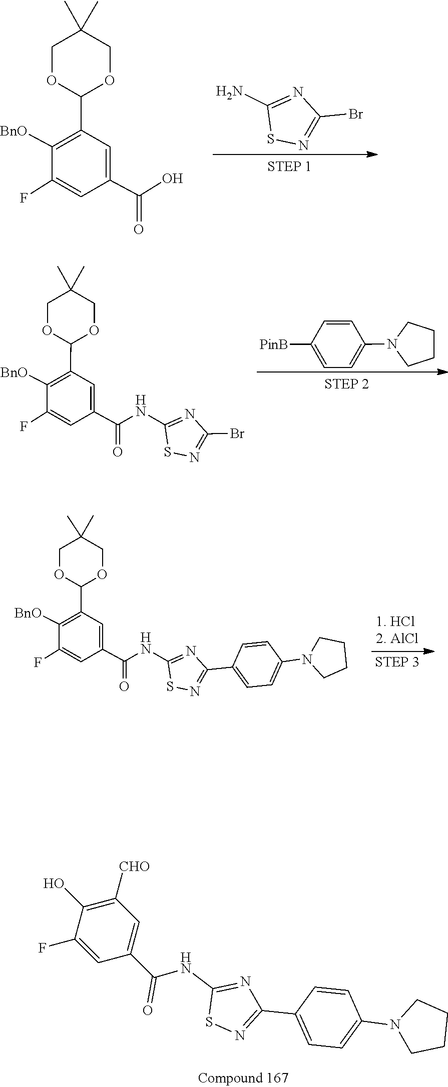

Step 1: DMF (1 drop) was added to a solution of 4-(benzyloxy)-3-(5,5-dimethyl-1,3-dioxan-2-yl)-5-fluorobenzoic acid (480 mg, 1.33 mmol, 1.2 eq.) and oxalyl chloride (500 mg, 3.94 mmol, 3 eq.) in DCM (5 mL) at 0° C. The reaction was stirred for 2 h at rt. The solvent was removed and the residue was co-evaporated with DCM two times. The residue was dissolved in DCM (2 mL) and added to a solution of 3-bromo-1,2,4-thiadiazol-5-amine (200 mg, 1.10 mmol, 1 eq.) and TEA (560 mg, 5.54 mmol, 5 eq.) in DCM (5 mL) at 0° C. The reaction was stirred for 1 h at rt. The solution was poured into sat. sodium bicarbonate solution and extracted with DCM three times. The organic extracts were combined, washed with brine, dried over anhydrous sodium sulfate, and concentrated. The resulting residue was purified by silica gel column chromatography (PE/EA=20:1 to 5:1) to give 4-(benzyloxy)-N-(3-bromo-1,2,4-thiadiazol-5-yl)-3-(5,5-dimethyl-1,3-dioxan-2-yl)-5-fluorobenzamide (302 mg, 0.58 mmol, 52% yield) as a white solid. LC-MS m/z [M+H]⁺ calc'd for C₂₂H₂₂BrFN₃O₄S 523, Found 523.

Step 2: In a 100 mL glass vial, Pd(dppf)₂Cl₂ (80 mg, 0.05 mmol, 0.1 eq.) was added to a mixture of 4-(benzyloxy)-N-(3-bromo-1,2,4-thiadiazol-5-yl)-3-(5,5-dimethyl-1,3-dioxan-2-yl)-5-fluorobenzamide (250 mg, 0.48 mmol, 1 eq.), 1-(4-(4,4,5,5-tetramethyl-1,3,2-dioxaborolan-2-yl)phenyl)pyrrolidine (261 mg, 0.96 mmol, 2 eq.), K2CO₃ (264 mg, 1.91 mmol, 4 eq.) in Dioxane/water (5 mL/15 mL). The reaction was heated overnight at 95° C. The reaction mixture was cooled to rt, poured into water, and extracted with ethyl acetate. The organic phase was dried over anhydrous sodium sulfate, and concentrated. The residue was purified by silica gel column chromatography (PE/EA=10:1 to 3:1) to give 4-(benzyloxy)-3-(5,5-dimethyl-1,3-dioxan-2-yl)-5-fluoro-N-(3-(4-(pyrrolidin-1-yl)phenyl)-1,2,4-thiadiazol-5-yl)benzamide (103 mg, 0.18 mmol, 36% yield). LC-MS m/z [M+H]⁺ calc'd for C₃₂H₃₄FN₄O₄S 589, Found 589.

Step 3: In a 50 mL galas vial, 4-(benzyloxy)-3-(5,5-dimethyl-1,3-dioxan-2-yl)-5-fluoro-N-(3-(4-(pyrrolidin-1-yl)phenyl)-1,2,4-thiadiazol-5-yl)benzamide (103 mg, 0.18 mmol, 1 eq.) was treated with 6N HCl/THF (3 mL/3 mL) for 2 h. The solution was extracted with ethyl acetate three times and DCM three times. The organic extracts were combined, washed with brine, dried over anhydrous sodium sulfate, and concentrated. The residue was dissolved in DCM (5 mL) and cooled to 0° C. AlCl3 (120 mg, 0.90 mmol, 5 eq.) was added and the reaction was stirred overnight at rt. The mixture was poured into water and pH was adjusted to 3-4. Then the mixture was extracted with DCM three times and ethyl acetate two times. The organic extracts were combined, dried over anhydrous sodium sulfate, and concentrated. The resulting residue was purified by silica gel column chromatography (PE/EA=10:1 to 2:1) and slurried in PE/DCM (10:1, 5 mL) to give 3-fluoro-5-formyl-4-hydroxy-N-(3-(4-(pyrrolidin-1-yl)phenyl)-1,2,4-thiadiazol-5-yl)benzamide (15 mg, 0.04 mmol, 20% yield) as a yellow solid. 1H NMR (CDCl₃, 400 MHz) δ: 9.79 (s, 1H), 8.02 (s, 1H), 7.95 (d, J=11.6 Hz, 1H), 7.88 (d, J=8.8 Hz, 2H), 6.55 (d, J=7.6 Hz, 2H), 3.34 (m, 4H), 2.08 (m, 4H). LC-MS m/z [M+H]⁺ calc'd for C₂₀H₁₈FN₄O₃S 413, found 413.

### Example 62: 3-fluoro-5-formyl-4-hydroxy-N-(2-(2-(pyrrolidin-1-yl)pyridin-4-yl)thiazol-5-yl)benzamide (Compound 168)

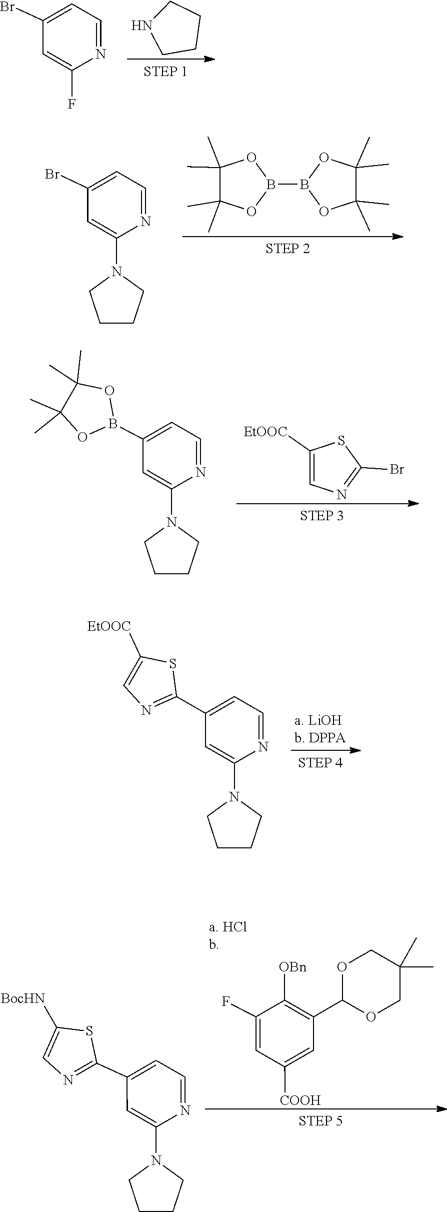

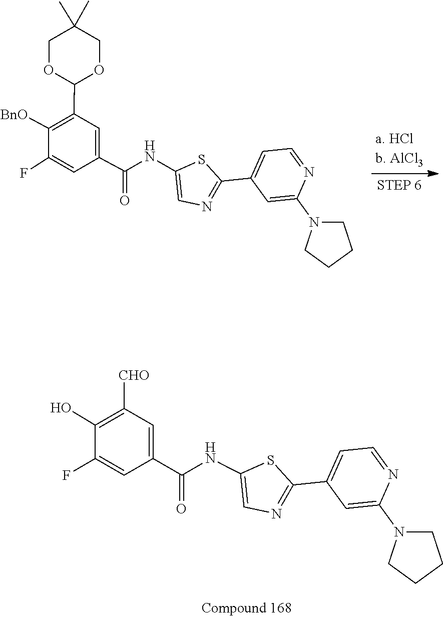 [SMILES: CC1(C)COC(c2cc(C(=O)Nc3cnc(-c4ccnc(N5CCCC5)c4)s3)cc(F)c2OCc2ccccc2)OC1 ; O=Cc1cc(C(=O)Nc2cnc(-c3ccnc(N4CCCC4)c3)s2)cc(F)c1O]

Step 1: A mixture of 4-bromo-2-fluoropyridine (5 g, 28.57 mmol, 1 eq.), pyrrolidine (2.2 g, 30.98 mmol, 1.1 eq.), and K₂CO₃ (11.8 g, 85.51 mmol, 3 eq.) in DMF (20 mL) was heated at 80° C. for 3 h. The mixture was poured into water and extracted with ethyl acetate three times. The organic extracts were combined, washed with brine, dried over anhydrous sodium sulfate, and concentrated. The residue was purified by silica gel column chromatography (PE/EA=200:1 to 100:1) to give 4-bromo-2-(pyrrolidin-1-yl)pyridine (5.03 g, 22.16 mmol, 78% yield). LC-MS m/z [M+H]⁺ calc'd for C₉H₁₁BrN₂ 227. Found 227.

Step 2: A solution of 4-bromo-2-(pyrrolidin-1-yl)pyridine (5 g, 22.12 mmol, 1 eq.), 4,4,4′,4′,5,5,5′,5′-octamethyl-2,2′-bi(1,3,2-dioxaborolane) (8.4 g, 33.19 mmol, 1.1 eq.), potassium acetate (6.5 g, 66.33 mmol, 3 eq.), and Pd(dppf)Cl₂ (1.62 g, 2.22 mmol, 0.1 eq.) in dioxane (30 mL) was heated at 110° C. for 2 h. The solution was cooled to rt and used for next reaction without work-up and purification.

Step 3: To the reaction solution of step-2, was added ethyl 2-bromothiazole-5-carboxylate (7.8 g, 33.18 mmol, 1.5 eq.), K₂CO₃ (9.2 g, 66.38 mmol, 3 eq.), and Pd(dppf)Cl₂ (1.62 g, 2.22 mmol, 0.1 eq.). The reaction was heated at 95° C. for 3 h. The mixture was poured into water and extracted with ethyl acetate three times. The organic extracts were combined, washed with brine, dried over anhydrous sodium sulfate, and concentrated. The residue was purified by silica gel column chromatography (PE/EA=100:1 to 10:1) to give ethyl 2-(2-(pyrrolidin-1-yl)pyridin-4-yl)thiazole-5-carboxylate (1.36 g, 4.49 mmol, 20% yield). LC-MS m/z [M+H]⁺ calc'd for C₁₅H₁₇N₃O₂S 304, found 304.

Step 4a: A solution of ethyl 2-(2-(pyrrolidin-1-yl)pyridin-4-yl)thiazole-5-carboxylate (1.36 g, 4.49 mmol, 1 eq.) and LiOH·H₂O (566 mg, 13.48 mmol, 3 eq.) in THF/water (5 mL/5 mL) was stirred for 2 h at rt. The reaction mixture was washed with PE. The water phase was acidified to pH=3 and the resulting precipitate was collected and co-evaporated with methanol to give the acid (1.2 g, 4.36 mmol, 97% yield) as a yellow solid.

Step 4b: The yellow solid was dissolved in t-BuOH (20 mL). DPPA (1.56 g, 5.67 mmol, 1.3 eq.) and TEA (705 mg, 6.98 mmol, 1.6 eq.) were added. The reaction was heated overnight at 75° C. The solution was cooled to rt, poured into water, acidified to pH=3, and extracted with ethyl acetate three times. The organic extracts were combined, washed with brine, dried over anhydrous sodium sulfate, and concentrated. The residue was purified by silica gel column chromatography (DCM/MeOH=50:1 to 30:1) to give tert-butyl 2-(2-(pyrrolidin-1-yl)pyridin-4-yl)thiazol-5-ylcarbamate (509 mg, 1.47 mmol, 34% yield). LC-MS m/z [M+H]⁺ calc'd for C₁₇H₂₂N₄O₂S 347, found 347.

Step 5a: In a 50 mL glass vial, tert-butyl 2-(2-(pyrrolidin-1-yl)pyridin-4-yl)thiazol-5-ylcarbamate (509 mg, 1.47 mmol, 1 eq.) was treated with 8 N HCl(gas)/dioxane (10 mL) for 2 h at rt. The reaction mixture was concentrated in vacuo and co-evaporated with DCM two times to give crude amine, which was used for next reaction without further purification.

Step 5b: In another vial, 4-(benzyloxy)-3-(5,5-dimethyl-1,3-dioxan-2-yl)-5-fluorobenzoic acid (189 mg, 0.53 mmol, 1.1 eq.) was mixed with TEA (53 mg, 0.53 mmol, 1.1 eq.) in DCM (10 mL). The solution was cooled to 0° C. and POCl₃ (81 mg, 0.53 mmol, 1.1 eq.) was added. The reaction was stirred for 30 min and the amine (135 mg, 0.48 mmol, 1 eq.) was added. The reaction was stirred for another 2 h at rt. The mixture was poured into water and extracted with ethyl acetate three times. The organic extracts were combined, dried over anhydrous sodium sulfate, and concentrated. The residue was purified by silica gel column chromatography (DCM/MeOH=50:1 to 20:1) to give 4-(benzyloxy)-3-(5,5-dimethyl-1,3-dioxan-2-yl)-5-fluoro-N-(2-(2-(pyrrolidin-1-yl)pyridin-4-yl)thiazol-5-yl)benzamide (163 mg, 0.28 mmol, 58% yield). LC-MS m/z [M+H]⁺ calc'd for C₃₂H₃₄FN₄O₄S 589, found, 589.

Step 6a: 4-(benzyloxy)-3-(5,5-dimethyl-1,3-dioxan-2-yl)-5-fluoro-N-(2-(2-(pyrrolidin-1-yl)pyridin-4-yl)thiazol-5-yl)benzamide (163 mg, 0.28 mmol, 1 eq.) was treated with THF/4N HCl (5 mL/5 mL) for 1 h at 50° C. The system was cooled to rt, neutralized with sodium bicarbonate, and extracted with ethyl acetate three times. The organic extracts were combined, washed with brine, dried over anhydrous sodium sulfate, and concentrated.

Step 6b: The residue was dissolved in DCM (10 mL) and anhydrous AlCl₃ (112 mg, 0.84 mmol, 3 eq.) was added. The reaction was stirred for 2 h at rt. The mixture was poured into water and acidified to pH=3. The mixture was then extracted with ethyl acetate three times. The organic extracts were combined, washed with brine, dried over anhydrous sodium sulfate, and concentrated. The residue was slurried in PE/DCM (10:1, 5 mL) three times to give 3-fluoro-5-formyl-4-hydroxy-N-(2-(2-(pyrrolidin-1-yl)pyridin-4-yl)thiazol-5-yl)benzamide (14 mg, 0.03 mmol, 12% yield) as a light yellow solid. ¹H NMR (CDCl₃, 400 MHz) δ: 11.42 (br, 1H), 10.14 (s, 1H), 8.11 (d, J=4.8 Hz, 1H), 8.01 (s, 1H), 7.78 (s, 1H), 7.49 (d, J=12.8 Hz, 1H), 6.97 (d, J=4.8 Hz, 1H), 6.87 (s, 1H), 6.80 (s, 1H), 3.44 (m, 4H), 1.97 (m, 4H). LC-MS m/z [M+H]⁺ calc'd for C₂₀H₁₈FN₄O₃S 413, found 413.

### Example 63: 5-(3-(4-(3,3-difluoroazetidin-1-yl)phenyl)-1,2,4-thiadiazol-5-yl)-3-fluoro-2-hydroxybenzaldehyde (Compound 169)

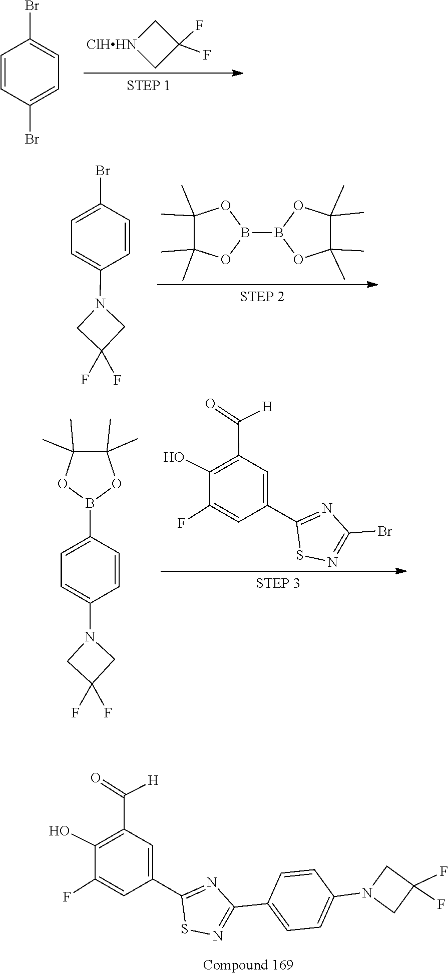

Step 1: A mixture of 1,4-dibromobenzene (1 g, 4.24 mmol, 1 eq.), 3,3-difluoroazetidine hydrochloride (604 mg, 4.66 mmol, 1.1 eq.), tBuONa (895 mg, 9.32 mmol, 2.2 eq.), Pd₂(dba)₃ (194 mg, 0.21 mmol, 0.05 eq.), and BINAP (263 mg, 0.42 mmol, 0.1 eq.) in toluene (20 mL) was heated at 85° C. for 3 h under N2 protection. Dioxane was removed in vacuo. The residue was dissolved in ethyl acetate/water (20 mL/20 mL). The organic extracts were combined, dried over anhydrous sodium sulfate, and concentrated. The residue was purified by silica gel column chromatography (PE) to give 1-(4-bromophenyl)-3,3-difluoroazetidine (344 mg, 1.39 mmol, 33% yield).

Step 2: A mixture of 1-(4-bromophenyl)-3,3-difluoroazetidine (319 mg, 1.29 mmol, 1 eq.), 4,4,4′,4′,5,5,5′,5′-octamethyl-2,2′-bi(1,3,2-dioxaborolane) (361 mg, 1.42 mmol, 1.1 eq.), potassium acetate (380 mg, 3.87 mmol, 3 eq.), PdCl₂(dppf) (94 mg, 0.13 mmol, 0.1 eq.) in dioxane (20 mL) was heated at 110° C. for 3 h under N2 protection. The solution was used for next reaction without work-up and purification.

Step 3: 5-(3-Bromo-1,2,4-thiadiazol-5-yl)-3-fluoro-2-hydroxybenzaldehyde (302 mg, 0.99 mmol, 1 eq.), potassium carbonate (411 mg, 2.98 mmol, 3 eq.), PdCl₂(dppf) (73 mg, 0.10 mmol, 0.1 eq.), and water (6 mL) were added to the reaction solution of Step 2 (1.38 mmol, 1.3 eq.). The system was degassed and heated at 95° C. for 2 h under N2 protection. Dioxane was removed in vacuo. pH of the water phase was adjusted to 4-5 and extracted with ethyl acetate three times. The organic extracts were combined, washed with brine, dried over anhydrous sodium sulfate, and concentrated. The residue was slurried in PE/DCM (10:1, 10 mL) three times to give 5-(3-(4-(3,3-difluoroazetidin-1-yl)phenyl)-1,2,4-thiadiazol-5-yl)-3-fluoro-2-hydroxybenzaldehyde (80 mg, 0.20 mmol, 21% yield) as a yellow solid ¹H NMR (DMSO-d6, 400 MHz) δ: 10.33 (s, 1H), 8.17 (d, J=8.4 Hz, 2H), 8.11 (s, 1H), 6.69 (d, J=8.8 Hz, 2H), 4.39 (t, J=12.4 Hz, 4H). LC-MS m/z [M−H]− calc'd for C₁₈H₁₁F₃N₃O₂S 390, found 390.0 (M−H)⁻).

### Example 64: 4-(5-(3-fluoro-5-formyl-4-hydroxyphenyl)-1,2,4-thiadiazol-3-yl)benzenesulfonamide (Compound 170)

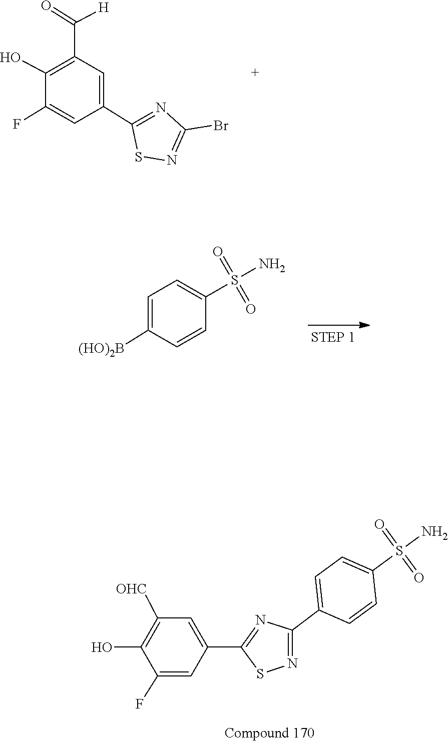 [SMILES: O=Cc1cc(-c2nc(Br)ns2)cc(F)c1O ; NS(=O)(=O)c1ccc(B(O)O)cc1 ; NS(=O)(=O)c1ccc(-c2nsc(-c3cc(F)c(O)c(C=O)c3)n2)cc1]

Step 1: A mixture of 5-(3-bromo-1,2,4-thiadiazol-5-yl)-3-fluoro-2-hydroxybenzaldehyde (56 mg, 0.18 mmol, 1 eq.), potassium carbonate (76 mg, 0.55 mmol, 3 eq.), PdCl₂(dppf) (14 mg, 0.02 mmol, 0.1 eq.), and 4-sulfamoylphenylboronic acid (40 mg, 0.20 mmol, 1.1 eq.) in dioxane/water (10 mL/3 mL) was heated at 95° C. for 2 h under N2 protection. The system was cooled to rt and the resulting precipitate was filtered. The cake was slurried in acetonitrile to give 4-(5-(3-fluoro-5-formyl-4-hydroxyphenyl)-1,2,4-thiadiazol-3-yl)benzenesulfonamide (23 mg, 0.06 mmol, 31% yield) as a yellow solid. ¹H NMR (DMSO-d6, 400 MHz) δ: 10.18 (s, 1H), 8.41 (d, J=8.4 Hz, 2H), 7.96 (d, J=8.4 Hz, 2H), 7.84 (d, J=2.4 Hz, 1H), 7.52 (dd, J=12.8 Hz, 2.0 Hz, 1H), 7.34 (br, 2H). LC-MS m/z [M+H]⁺ calc'd for C₁₅H₁₁FN₃O₄S₂ 380, found 380.

### Example 65: 3-fluoro-2-hydroxy-5-(3-(4-(pyrrolidin-1-yl)phenyl)-1,2,4-thiadiazol-5-yl)benzaldehyde oxime (Compound 171)

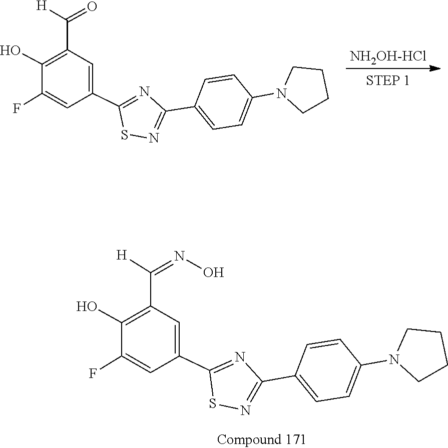

Step 1:Heated 3-fluoro-2-hydroxy-5-(3-(4-(pyrrolidin-1-yl)phenyl)-1,2,4-thiadiazol-5-yl)benzaldehyde (200 mg, 0.54 mmol) with hydroxylamine hydrochloride (53 mg) and potassium acetate (53 mg) in 70% EtOH/water to 80° C. for 2-3 h. The reaction was cooled and stirred overnight. The solids were filtered off and washed successively with hexanes, 70% EtOH in water, and hexanes. The solid was frozen and placed under high vacuum to provide the title compound, 3-fluoro-2-hydroxy-5-(3-(4-(pyrrolidin-1-yl)phenyl)-1,2,4-thiadiazol-5-yl)benzaldehyde oxime as a yellow solid (194.4 mg, 0.5 mmol, 94% yield). LC-MS m/z [M+H]⁺ calc'd for C₁₉H₁₈FN₄O₂S 385, found 385.

### Example 66: 2-fluoro-6-formyl-4-(3-(4-(pyrrolidin-1-yl)phenyl)-1,2,4-thiadiazol-5-yl)phenyl pyrrolidine-1-carboxylate (Compound 172)

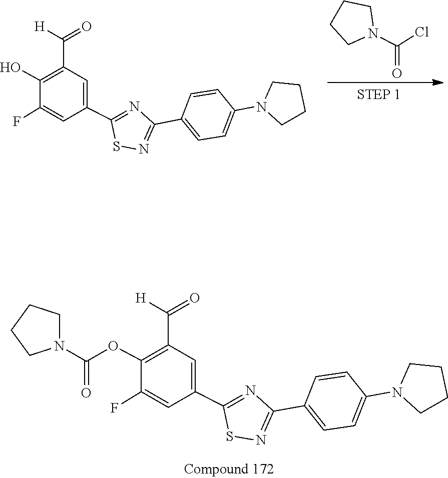 [SMILES: O=Cc1cc(-c2nc(-c3ccc(N4CCCC4)cc3)ns2)cc(F)c1O ; O=C(Cl)N1CCCC1 ; O=Cc1cc(-c2nc(-c3ccc(N4CCCC4)cc3)ns2)cc(F)c1OC(=O)N1CCCC1]

Step 1: 3-fluoro-2-hydroxy-5-(3-(4-(pyrrolidin-1-yl)phenyl)-1,2,4-thiadiazol-5-yl)benzaldehyde (200 mg, 0.54 mmol, 1.0 equiv) was dissolved in DCM. Triethylamine (0.754 mL) and 4-dimethylaminopyridine (83 mg) were added and the mixture was stirred at room temperature for a few minutes, after which pyrrolidine-1-carbonyl chloride (0.3 mL, 2.7 mmol, 5 eq.) was added dropwise. The mixture was stirred for 16 h at room temperature. The reaction mixture was quenched by H2O (10 mL) and diluted in 100 mL of DCM. The mixture was successively washed with saturated sodium bicarbonate, 0.5N HCl and brine, then dried over anhydrous Na₂SO₄. After filtration, the solution was concentrated under reduced pressure. The residue was purified by column chromatography on silica gel (0-5% MeOH/DCM) to give the title compound, 2-fluoro-6-formyl-4-(3-(4-(pyrrolidin-1-yl)phenyl)-1,2,4-thiadiazol-5-yl)phenyl pyrrolidine-1-carboxylate (156.4 mg, 0.33 mmol, 62% yield). ¹H NMR (500 MHz, CDCl₃) δ 10.25 (s, 1H), 8.28-8.19 (m, 3H), 8.15 (dd, J=9.9, 2.1 Hz, 1H), 6.68-6.53 (m, 2H), 3.69 (t, J=6.7 Hz, 2H), 3.55 (t, J=6.7 Hz, 2H), 3.44-3.32 (m, 4H), 2.08-1.97 (m, 8H). LC-MS m/z [M+H]⁺ calc'd for C₂₄H₂₄FN₄O₃S 467, found 467.

### Example 67: 2-fluoro-6-formyl-4-(3-(4-(pyrrolidin-1-yl)phenyl)-1,2,4-thiadiazol-5-yl)phenyl dimethylcarbamate (Compound 173)

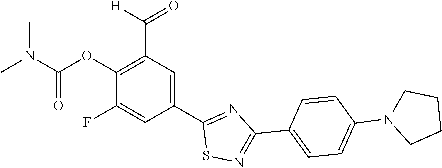 [SMILES: CN(C)C(=O)Oc1c(F)cc(-c2nc(-c3ccc(N4CCCC4)cc3)ns2)cc1C=O]

The title compound was synthesized in the same manner as Example 66 using dimethylcarbamic chloride to give 2-fluoro-6-formyl-4-(3-(4-(pyrrolidin-1-yl)phenyl)-1,2,4-thiadiazol-5-yl)phenyl dimethylcarbamate (200.3 mg, 84% yield). ¹H NMR (500 MHz, CDCl₃) δ 10.23 (s, 1H), 8.27-8.19 (m, 3H), 8.15 (dd, J=9.9, 2.1 Hz, 1H), 6.66-6.59 (m, 2H), 3.42-3.33 (m, 4H), 3.22 (s, 3H), 3.09 (s, 3H), 2.09-2.00 (m, 4H). LC-MS m/z [M+H]⁺ calc'd for C₂₂H₂₂FN₄O₃S 441, found 441.

### Example 68: 2-fluoro-6-formyl-4-(3-(4-(pyrrolidin-1-yl)phenyl)-1,2,4-thiadiazol-5-yl)phenyl morpholine-4-carboxylate (Compound 174)

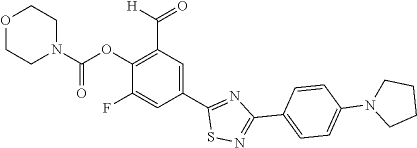 [SMILES: O=Cc1cc(-c2nc(-c3ccc(N4CCCC4)cc3)ns2)cc(F)c1OC(=O)N1CCOCC1]

The title compound was prepared using a method similar to that as described in Example 67 using morpholine-4-carbonyl chloride to give 2-fluoro-6-formyl-4-(3-(4-(pyrrolidin-1-yl)phenyl)-1,2,4-thiadiazol-5-yl)phenyl morpholine-4-carboxylate (242 mg, 90% yield). ¹H NMR (500 MHz, CDCl₃) δ 10.20 (s, 1H), 8.28-8.18 (m, 3H), 8.15 (dd, J=9.8, 2.1 Hz, 1H), 6.66-6.59 (m, 2H), 3.84-3.78 (m, 6H), 3.63 (t, J=4.6 Hz, 2H), 3.43-3.35 (m, 4H), 2.10-2.00 (m, 4H). LC-MS m/z [M+H]⁺ calc'd for C₂₄H₂₄FN₄O₄S 483, found 483.

### Example 69: 2-fluoro-6-formyl-4-(3-(4-(pyrrolidin-1-yl)phenyl)-1,2,4-thiadiazol-5-yl)phenyl 4-methylpiperazine-1-carboxylate (Compound 175)

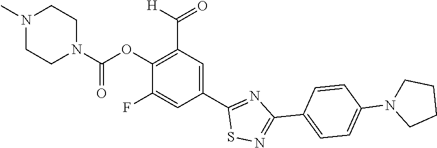 [SMILES: CN1CCN(C(=O)Oc2c(F)cc(-c3nc(-c4ccc(N5CCCC5)cc4)ns3)cc2C=O)CC1]

The title compound was prepared using a method similar to that as described in Example 67 using 4-methylpiperazine-1-carbonyl chloride to give 2-fluoro-6-formyl-4-(3-(4-(pyrrolidin-1-yl)phenyl)-1,2,4-thiadiazol-5-yl)phenyl 4-methylpiperazine-1-carboxylate (220 mg, 86% yield). 1H NMR (500 MHz, CDCl₃) δ 10.22 (s, 1H), 8.29-8.18 (m, 3H), 8.15 (dd, J=9.8, 2.1 Hz, 1H), 6.73-6.50 (m, 2H), 3.81 (d, J=5.4 Hz, 2H), 3.64 (s, 2H), 3.49-3.28 (m, 4H), 2.64-2.45 (m, 4H), 2.38 (s, 3H), 2.15-1.94 (m, 4H). LC-MS m/z [M+H]⁺ calc'd for C₂₅H₂₇FN₅O₃S 496, found 496.

### Example 70: 2-fluoro-6-formyl-4-(3-(4-(pyrrolidin-1-yl)phenyl)-1,2,4-thiadiazol-5-yl)phenyl 2-(2-methoxyethoxy)acetate (Compound 176)

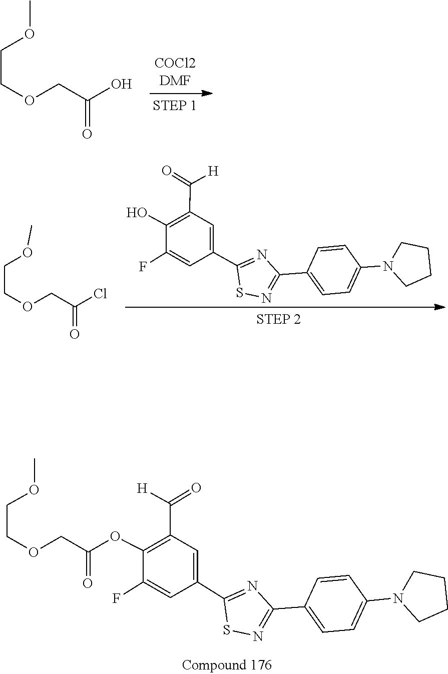

Step 1: DMF (1 drop) was added to a solution of 2-(2-methoxyethoxy)acetic acid (182 mg, 1.36 mmol, 1 eq.) and oxalyl chloride (344 mg, 2.71 mmol, 2 eq.) in DCM (5 mL) at 0° C. The reaction was stirred for 2 h at rt. The solvent was removed and the residue was co-evaporated with DCM two times. The residue, 2-(2-methoxyethoxy)acetyl chloride, (1.36 mmol, quantitative yield) was used for next reaction without further purification.

Step 2: The residue in Step 1 (1.36 mmol, 5.0 eq.) was dissolved in DCM (10 mL). The solution was cooled to 0° C. A solution of 3-fluoro-2-hydroxy-5-(3-(4-(pyrrolidin-1-yl)phenyl)-1,2,4-thiadiazol-5-yl)benzaldehyde (100 mg, 0.27 mmol, 1 eq.) in DCM (2 mL) and TEA (219 mg, 2.17 mmol, 8 eq.) were added successively. The reaction was stirred overnight at rt. The system was poured into cooled acidic (KHSO₄) water and extracted with DCM two times. The organic extracts were combined, dried over anhydrous sodium sulfate, and concentrated. The residue was slurried in PE/DCM (10:1, 5 mL) three times to give 2-fluoro-6-formyl-4-(3-(4-(pyrrolidin-1-yl)phenyl)-1,2,4-thiadiazol-5-yl)phenyl 2-(2-methoxyethoxy)acetate (20 mg, 0.04 mmol, 15% yield) as a yellow solid. ¹H NMR (CDCl₃, 400 MHz) δ: 10.16 (s, 1H), 8.24 (m, 3H), 8.17 (d, J=9.6 Hz, 1H), 6.71 (d, J=8.0 Hz, 2H), 4.62 (s, 2H), 3.88 (m, 2H), 3.65 (m, 2H), 3.45 (m, 4H), 2.08 (m, 4H). LC-MS m/z [M+H]⁺ calc'd for C₂₄H₂₄FN₃O₅S 486, found: 486.

### Example 71: 2-(2-fluoro-6-formyl-4-(3-(4-(pyrrolidin-1-yl)phenyl)-1,2,4-thiadiazol-5-yl)phenoxy)-1-methylpyridinium iodide (Compound 177)

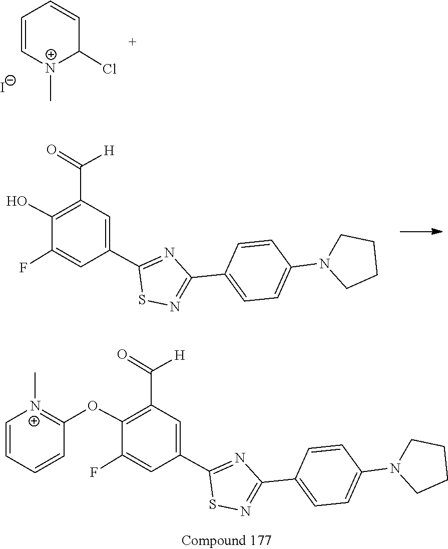

A solution of 2-chloro-1-methylpyridinium iodide (138 mg, 0.54 mmol, 2 eq.), TEA (82 mg, 0.81 mmol, 3 eq.), and 3-fluoro-2-hydroxy-5-(3-(4-(pyrrolidin-1-yl)phenyl)-1,2,4-thiadiazol-5-yl)benzaldehyde (100 mg, 0.27 mmol, 1 eq.) in ACN/DMSO (10 mL/1 mL) was heated at 40° C. for 30 min. The resulting precipitate was filtered off and the filtrate was concentrated in vacuo. The residue was slurried in ACN two times to give 2-(2-fluoro-6-formyl-4-(3-(4-(pyrrolidin-1-yl)phenyl)-1,2,4-thiadiazol-5-yl)phenoxy)-1-methylpyridinium iodide (50 mg, 0.09 mmol, 32% yield) as a yellow solid ¹H NMR (DMSO-d6, 400 MHz) δ: 10.18 (s, 1H), 8.99 (d, J=6.0 Hz, 1H), 8.71 (dd, J=10.4 Hz, 1.6 Hz, 1H), 8.65 (s, 1H), 8.52 (s, 1H), 8.15 (d, J=8.8 Hz, 1H), 7.86 (m, 1H), 7.66 (d, J=8.8 Hz, 1H), 6.68 (d, J=8.8 Hz, 2H), 4.36 (s, 3H), 3.34 (m, 4H), 1.99 (m, 4H). LC-MS m/z [M+H]⁺ calc'd for C₂₅H₂₃FN₄O₂S⁺I⁻ 462, found 462.

### Example 72: 8-fluoro-4-hydroxy-3-methyl-6-(3-(4-(pyrrolidin-1-yl)phenyl)-1,2,4-thiadiazol-5-yl)-3,4-dihydro-2H-benzo[e][1,3]oxazin-2-one (Compound 178)

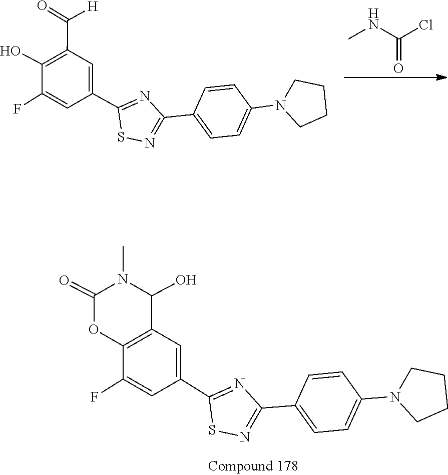

Methylcarbamic chloride (1.01 g, 10.80 mmol, 10.0 eq.) was added to a solution of 3-fluoro-2-hydroxy-5-(3-(4-(pyrrolidin-1-yl)phenyl)-1,2,4-thiadiazol-5-yl)benzaldehyde (400 mg, 1.08 mmol, 1 eq.) and DIEA (1.68 g, 13.02 mmol, 12 eq.) in DCM (20 mL). The reaction was stirred at rt for 1 h. The solution was concentrated in vacuo. The residue was slurried in THF (−20 mL) and the resulting precipitate was filtered off. The filtrate was concentrated in vacuo and the residue was stirred in PE/DCM (2:1, ˜15 mL). Two layers were formed. The lower layer was collected and stirred in DCM (−5 mL). The resulting precipitate was collected by filtration and slurried in DCM (−5 mL) again to give 8-fluoro-4-hydroxy-3-methyl-6-(3-(4-(pyrrolidin-1-yl)phenyl)-1,2,4-thiadiazol-5-yl)-3,4-dihydro-2H-benzo[e][1,3]oxazin-2-one (75 mg, 0.18 mmol, 16% yield) as a yellow solid. ¹H NMR (DMSO-d6, 400 MHz) δ: 8.11 (m, 3H), 7.96 (s, 1H), 7.12 (d, J=8.8 Hz, 1H), 6.65 (d, J=8.8 Hz, 2H), 5.93 (d, J=8.8 Hz, 1H), 3.32 (m, 4H), 3.09 (s, 3H), 1.99 (m, 4H). LC-MS m/z [M+H]⁺ calc'd for C₂₁H₂₀FN₄O₃S 427, found 427.

### Example 73: 8-fluoro-4-hydroxy-3-(2-methoxyethyl)-6-(3-(4-(pyrrolidin-1-yl)phenyl)-1,2,4-thiadiazol-5-yl)-3,4-dihydro-2H-benzo[e][1,3]oxazin-2-one (Compound 179)

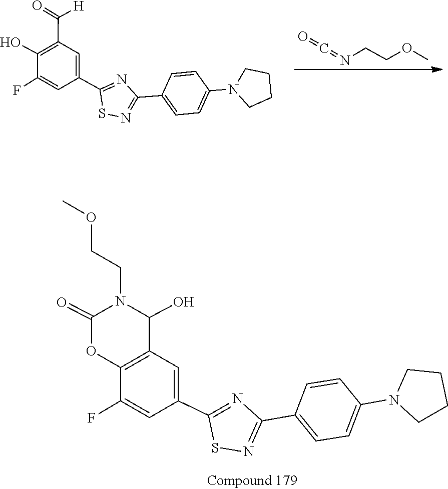

Step 1: 3-fluoro-2-hydroxy-5-(3-(4-(pyrrolidin-1-yl)phenyl)-1,2,4-thiadiazol-5-yl)benzaldehyde (200 mg, 0.54 mmol, 1.0 equiv) was dissolved in DCM. Triethylamine (0.226 mL) was added and the mixture was stirred at room temperature for a few minutes, after which 1-isocyanato-2-methoxyethane (0.3 mL, 2.7 mmol, 5 eq.) was added dropwise. The mixture was stirred for 16 h at room temperature. The reaction mixture was quenched with water (10 mL) and diluted in 100 mL of DCM. The mixture was successively washed with saturated sodium bicarbonate, 0.5N HCl and brine, then dried over anhydrous Na₂SO₄. After filtration, the solution was concentrated under reduced pressure. The residue was precipitated from DCM/Hexanes 1:1 to give 19.6 mg of 8-fluoro-4-hydroxy-3-(2-methoxyethyl)-6-(3-(4-(pyrrolidin-1-yl)phenyl)-1,2,4-thiadiazol-5-yl)-3,4-dihydro-2H-benzo[e][1,3]oxazin-2-one in 8% yield. ¹H NMR (500 MHz, CDCl3) δ 8.24-8.17 (m, 2H), 7.89 (dd, J=10.2, 1.9 Hz, 1H), 7.79 (s, 1H), 6.65-6.58 (m, 2H), 5.80 (d, J=2.2 Hz, 1H), 5.70 (d, J=2.3 Hz, 1H), 4.28 (ddd, J=15.2, 3.1, 1.7 Hz, 1H), 3.93 (td, J=10.4, 1.7 Hz, 1H), 3.63 (dt, J=10.4, 2.7 Hz, 1H), 3.52 (s, 3H), 3.41-3.34 (m, 4H), 3.31 (ddd, J=15.2, 10.4, 2.4 Hz, 1H), 2.09-2.00 (m, 4H). LC-MS m/z [M+H]⁺ calc'd for C₂₃H₂₄FN₄O₄S 471, found 471.

### Example 74: 3-fluoro-2-hydroxy-5-(3-(4-(pyrrolidin-1-yl)phenyl)morpholine-4-carbonyl)benzaldehyde (Compound 180)

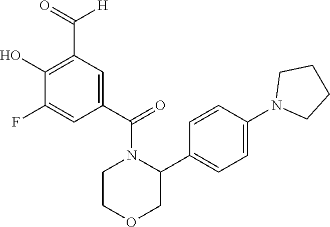 [SMILES: O=Cc1cc(C(=O)N2CCOCC2c2ccc(N3CCCC3)cc2)cc(F)c1O]

The title compound was prepared using a method similar to that as described in Example 41 starting from 3-(4-bromophenyl)morpholine hydrochloride and 3-fluoro-5-formyl-4-hydroxybenzoic acid to provide 3-fluoro-2-hydroxy-5-(3-(4-(pyrrolidin-1-yl)phenyl)morpholine-4-carbonyl)benzaldehyde. LCMS m/z [M+H]⁺ calc'd for C₂₂H₂₄FN₂O₄ 399, found 399.

### Example 75: 3-fluoro-2-hydroxy-5-(3-(3-(pyrrolidin-1-yl)phenyl)morpholine-4-carbonyl)benzaldehyde (Compound 181)

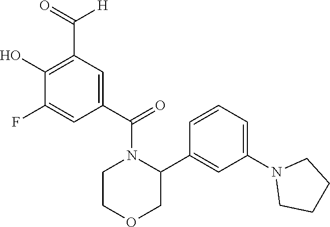 [SMILES: O=Cc1cc(C(=O)N2CCOCC2c2cccc(N3CCCC3)c2)cc(F)c1O]

The title compound was prepared using a method similar to that as described in Example 41 starting from 3-(3-bromophenyl)morpholine hydrochloride and 3-fluoro-5-formyl-4-hydroxybenzoic acid to provide 3-fluoro-2-hydroxy-5-(3-(3-(pyrrolidin-1-yl)phenyl)morpholine-4-carbonyl)benzaldehyde. LCMS m/z [M+H]⁺ calc'd for C₂₂H₂₄FN₂O₄ 399, found 399.

### Example 76: 3-fluoro-2-hydroxy-5-(3-(3-(pyrrolidin-1-yl)phenyl)thiomorpholine-4-carbonyl)benzaldehyde (Compound 182)

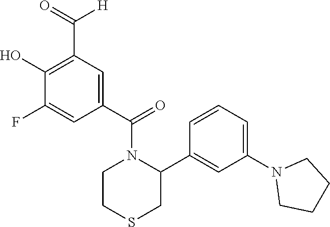 [SMILES: O=Cc1cc(C(=O)N2CCSCC2c2cccc(N3CCCC3)c2)cc(F)c1O]

The title compound was prepared using a method similar to that as described in Example 41 starting from 3-(3-chlorophenyl)thiomorpholine and 3-fluoro-5-formyl-4-hydroxybenzoic acid to provide 3-fluoro-2-hydroxy-5-(3-(3-(pyrrolidin-1-yl)phenyl)thiomorpholine-4-carbonyl)benzaldehyde. LCMS m/z [M+H]⁺ calc'd for C₂₂H₂₄FN₂O₃S 415, found 415.

### Example 77: 3-fluoro-5-formyl-4-hydroxy-N′-(4-(pyrrolidin-1-yl)benzoyl)benzohydrazide (Compound 183)

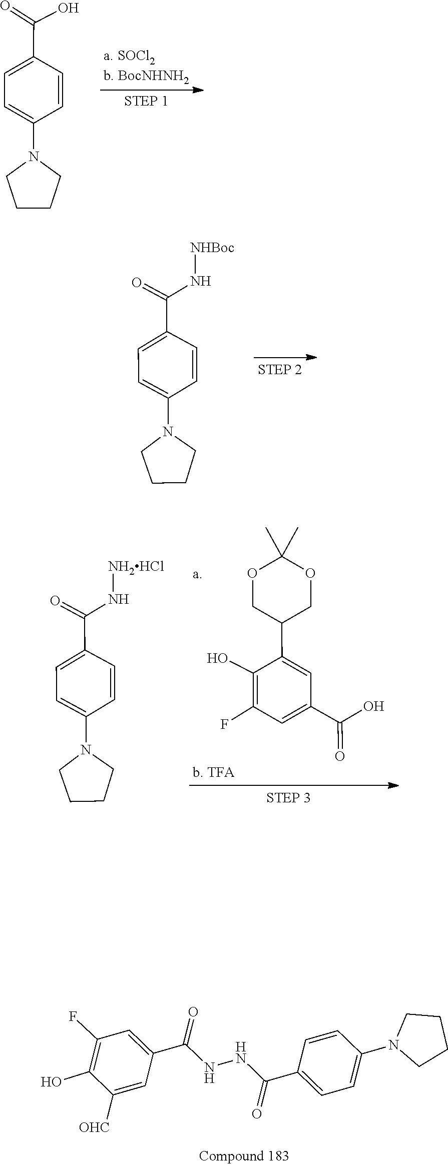

Step 1a: A solution of 4-(pyrrolidin-1-yl)benzoic acid (1 g, 5.24 mmol, 1 eq.) and thionyl chloride (2 mL) in DCM (10 mL) was heated at 40° C. for 2 h under N2 protection. The solvent was removed in vacuo and the residue was co-evaporated with DCM two times.

Step 1b: The residue was dissolved in THF (10 mL) and cooled to 0° C. Boc-hydrazine (690 mg, 5.23 mmol) and TEA (1.06 g, 10.46 mmol) were added. The reaction was stirred overnight at rt. The solution was poured into sat. sodium bicarbonate (30 mL) and extracted with ethyl acetate three times. The organic extracts were combined, washed with brine, dried over anhydrous sodium sulfate, and concentrated. The residue was purified by silica gel column (PE/EA=200:1 to 4:1) to give tert-butyl 2-(4-(pyrrolidin-1-yl)benzoyl)hydrazinecarboxylate (710 mg, 2.33 mmol, 44.5% yield). LC-MS m/z [M+H]⁺ calc'd for C₁₆H₂₄N₃O₃. 306, found 306.

Step 2: In a 100 mL glass vial, tert-butyl 2-(4-(pyrrolidin-1-yl)benzoyl)hydrazinecarboxylate (710 g, 2.33 mmol, 1 eq.) was dissolved in DCM (5 mL) and 6N HCl(gas)/dioxane (1.5 mL) was added. The reaction was stirred for 2 h. The solvent was removed in vacuo and the residue was co-evaporated with DCM two times to give crude 4-(pyrrolidin-1-yl)benzohydrazide hydrochloride (680 mg, 2.33 mmol, quantitative yield), which was used for next reaction without further purification.

Step 3a: In a 100 mL glass vial, HATU (1.06 g, 2.78 mmol, 1.2 eq.) was added to a solution of 4-(pyrrolidin-1-yl)benzohydrazide hydrochloride (680 mg, 2.33 mmol, 1 eq.), 3-(2,2-dimethyl-1,3-dioxan-5-yl)-5-fluoro-4-hydroxybenzoic acid (661 g, 2.45 mmol, 1.05 eq.), and DIEA (897 mg, 6.96 mmol, 3 eq.) in DMF (10 mL). The reaction was stirred overnight at rt. The reaction solution was poured into sat. sodium bicarbonate and extracted with ethyl acetate three times. The organic extracts were combined, washed with brine, dried over anhydrous sodium sulfate, and concentrated. The residue was purified by silica gel column chromatography (DCM/THF=100:1 to 80:1) to give the intermediate (crude, 330 mg, 0.72 mmol, 31.0% yield).

Step 3b: The intermediate (160 mg, 0.35 mmol, 1 eq.) was mixed with TFA (6 mL) in DCM/THF/water (8 mL/1 mL/4 mL). The reaction was stirred for 1.5 h. The system was poured slowly into ice-cold saturated sodium bicarbonate solution and the mixture was stirred for 30 min. The resulting precipitate was filtered, slurried in DCM two times. The solid was collected by filtration to give 3-fluoro-5-formyl-4-hydroxy-N′-(4-(pyrrolidin-1-yl)benzoyl)benzohydrazide (80.7 mg, 0.19 mmol, 54.8% yield) as a yellow-green solid. ¹H NMR (DMSO-d6, 400 MHz) δ: 11.17 (br, 1H), 10.43 (s, 1H), 10.34 (s, 1H), 10.11 (s, 1H), 8.14 (s, 1H), 8.00 (d, J=11.2 Hz, 1H), 7.79 (d, J=8.4 Hz, 2H), 6.57 (d, J=8.4 Hz, 2H), 3.30 (m, 4H), 1.97 (m, 4H). LC-MS m/z [M+H]⁺ calc'd for C₁₉H₁₉FN₃O₄ 372, found 372.

### Example 78: 3-fluoro-2-hydroxy-5-(5-(pyrrolidin-1-yl)-2,3-dihydrospiro[indene-1,4′-piperidine]-1′-carbonyl)benzaldehyde (Compound 192)

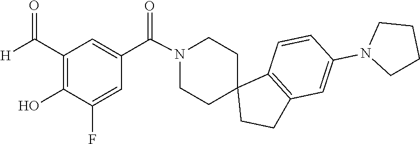 [SMILES: O=Cc1cc(C(=O)N2CCC3(CCc4cc(N5CCCC5)ccc43)CC2)cc(F)c1O]

The title compound was prepared using a method similar to that as described in Example 49 starting from tert-butyl 5-bromo-2,3-dihydrospiro[indene-1,4′-piperidine]-1′-carboxylate and 3-fluoro-5-formyl-4-hydroxybenzoic acid to provide 3-fluoro-2-hydroxy-5-(5-(pyrrolidin-1-yl)-2,3-dihydrospiro[indene-1,4′-piperidine]-1′-carbonyl)benzaldehyde. LCMS m/z [M+H]⁺ calc'd for C₂₅H₂₈FN₂O₃ 423, found 423.

### Biological Example 1: In Vitro Assay

i. TLR2 Assays

Synthetic diacylated lipoprotein (Pam2CSK4, TLR2/6 agonist) and synthetic triacylated lipoprotein (Pam3CSK4, TLR1/2 agonist) were obtained from InvivoGen and were dissolved in endotoxin-free water to a concentration 1 mg/mL, vortexed until complete solubilization, and stored in aliquots at −20° C. Prior to addition to cells, an aliquot of the dissolved ligand was vortexed shortly and then was diluted in medium to 25 ng/mL Pam2CSK4 or 1000 ng/mL Pam3CSK4. The EC₅₀ of the agonists for each assay run was determined by using 3-fold dilutions of each agonist from the following starting concentrations: 5 ng/mL for Pam2CSK4, and 200 ng/mL for Pam3CSK4.

Test compounds were solubilized fresh to 10-20 mM stocks in DMSO and sonicated for 5-10 minutes in a water bath sonicator. Serial dilutions were prepared in DMSO, and then diluted in medium. The final concentration of DMSO used in the assay was 1%.

HEK-Blue hTLR2 reporter cells (InvivoGen) are HEK-293 cells stably expressing both the human TLR2 gene and a secreted embryonic alkaline phosphatase (SEAP) reporter construct downstream of NFκB promotor sites. HEK-Blue hTLR2 reporters were cultured according to manufacturer's protocol using Dulbecco's Modified Eagle Medium (DMEM; Gibco) containing 1× GlutaMax (Gibco), 10% heat-inactivated Fetal Bovine Serum (Gibco), Pen-Strep (50 U/mL penicillin, 50 μg/mL streptomycin, Gibco), 100 μg/mL Normocin (InvivoGen), and the selective antibiotic, lx HEK-Blue Selection (InvivoGen). Quanti-Blue reagent (InvivoGen) for detection and quantification of secreted alkaline phosphatase was dissolved in 100 mL of endotoxin-free water, warmed to 37° C. for 30 minutes and then filtered using a 0.2 μm membrane.

ii. TLR9 Assay

Synthetic ODNs (ODN 2006 (ODN 7909), class B CpG oligonucleotide, TLR9 agonist) was obtained from InvivoGen and was dissolved in endotoxin-free water to a concentration 500 μM, vortexed until complete solubilization, and stored in aliquots at −20° C. Prior to addition to cells, an aliquot of the dissolved ligand was vortexed shortly and then was diluted in medium to 50 μM. The EC₅₀ of the agonist for each assay run was determined by using 3-fold dilutions from the starting concentration 10 PM.

Test compounds were solubilized fresh to 10-20 mM stocks in DMSO and sonicated for 5-10 minutes in a water bath sonicator. Serial dilutions were prepared in DMSO, and then diluted in medium. The final concentration of DMSO used in the assay was 1%.

HEK-Blue hTLR9 reporter cells (InvivoGen) are HEK-293 cells stably expressing both the human TLR9 gene and a secreted embryonic alkaline phosphatase (SEAP) reporter construct downstream of NFκB promotor sites. HEK-Blue hTLR9 cells were cultured according to manufacturer's protocol using Dulbecco's Modified Eagle Medium (DMEM; Gibco) containing 1× GlutaMax (Gibco), 10% heat-inactivated Fetal Bovine Serum (Gibco), Pen-Strep (50 U/mL penicillin, 50 μg/mL streptomycin, Gibco), 100 μg/mL Normocin (InvivoGen), and the selective antibiotics, 10 μg/mL Blasticidin (InvivoGen), and 100 μg/mL Zeocin (InvivoGen). Quanti-Blue reagent (InvivoGen) for detection and quantification of secreted alkaline phosphatase was dissolved in 100 mL of endotoxin-free water, warmed to 37° C. for 30 minutes and then filtered using a 0.2 μm membrane.

### Biological Example 2: HEK-Blue hTLR2 Antagonism Assay

i. TLR2 Assays

On day 1, 50 μL of each test compound dilution in duplicates or a vehicle control was added to each well of a 96-well plate followed by addition of 150 μL of HEK-Blue hTLR2 cell suspension (1×10⁵ cells/well) and incubated at 37° C./5% CO₂ for 2 h. Next, 50 μL of an approximate 3×EC₅₀ concentration of each agonist (Pam2CSK4 or Pam3CSK4) was added to the wells containing test compounds or the vehicle control. The plates were then incubated at 37° C./5% CO₂ for 18 h. For each assay run, non-treated HEK-Blue hTLR2 cells were treated with serial dilutions of agonists to determine EC₅₀ values for the respective run.

On day 2, secreted alkaline phosphatase (SEAP) activity was detected in cell culture supernatants. In brief, 20 μL was collected from each well and transferred to a 96-well plate. Next, 200 μL of Quanti-Blue detection reagent was added to each well. Plates were incubated at room temperature for 15 min and SEAP activity was assessed by spectrophotometer OD reading at 655 nm. Table A shows the activities of the compounds tested in HEK cells using Pam2CSK4 and Pam3CSK4 as agonists. The activities of the compounds against Pam2CSK4 and Pam3CSK4 are presented as IC₅₀ values which were defined as concentrations of the compounds where percent inhibition of the signal induced by agonist is equal to 50%. IC₅₀ values were calculated based on 8-point dilutions for each compound.

ii. TLR9 Assay

On day 1, 50 μL of each test compound dilution in duplicates or a vehicle control was added to each well of a 96-well plate followed by addition of 150 μL of HEK-Blue hTLR9 cell suspension (1×10⁵ cells/well) and incubated at 37° C./5% CO₂ for 2 h. Next, 50 μL of an approximate 3×EC₅₀ concentration of TLR9 agonist, ODN 2006, was added to the wells containing test compounds or the vehicle control. The plates were then incubated at 37° C./5% CO₂ for 18 h. For each assay run, vehicle-treated HEK-Blue hTLR9 cells were treated with serial dilutions of agonist to determine EC₅₀ values for the respective run.

On day 2, secreted alkaline phosphatase (SEAP) activity was detected in cell culture supernatants. In brief, 30 μL was collected from each well and transferred to a 96-well plate. Next, 200 μL of Quanti-Blue detection reagent was added to each well. Plates were incubated at 37° C. for 60 min. and SEAP activity was assessed by spectrophotometer OD reading at 655 nm. Table A shows the activities of the compounds tested in HEK-Blue hTLR9 cells against ODN 2006. The activities of the compounds against ODN 2006 are presented as IC₅₀ values which were defined as concentrations of the compounds where percent inhibition of the signal induced by agonist is equal to 50%. Exact IC₅₀ values were calculated based on 8-point dilutions for each compound. Approximate IC₅₀ values (˜ or <) were calculated based on 4-point dilutions for each compound.

TABLE A

| Example | Compound | IC₅₀ (μM) with | IC₅₀ (μM) with | IC₅₀ (μM) with |
|---|---|---|---|---|
| No. | No. | Pam2CSK4 | Pam3CSK4 | ODN2006 |
| 1 | 1 | 45.8 | 21.8 | ND |
| 2 | 2 | 6.7 | 2.2 | ~0.5 |
| 3 | 3 | 2.3 | 1.7 | ND |
| 4 | 4 | 0.9 | 0.5 | 0.2 |
| 5 | 5 | 5.3 | 2.7 | ND |
| 6 | 6 | 5.1 | 3.3 | ND |
| 7 | 7 | >100 | >100 | >33 |
| 8 | 8 | 17.7 | 12.9 | ND |
| 9 | 9 | 5.2 | 5.2 | ND |
| 10 | 10 | 1.6 | 0.9 | ~1 |
| 11 | 11 | 1.0 | 0.7 | 0.4 |
| 12 | 12 | 0.6 | 0.6 | 0.2 |
| 13 | 13 | 1.5 | 1.0 | ND |
| 14 | 14 | 18.2 | 18.7 | ND |
| 15 | 15 | 4.1 | 0.9 | ND |
| 16 | 18 | 22.2 | 20.3 | ND |
| 17 | 19 | 0.4 | 0.3 | 0.6 |
| 18 | 34 | 0.9 | 0.4 | ND |
| 19 | 35 | 13.8 | 8 | ND |
| 38 | 36 | 17.40 | 15.10 | ND |
| 37 | 37 | 6.20 | 4.80 | ND |
| 20 | 47 | ND | ND | ND |
| 21 | 82 | 8.4 | 3.5 | ND |
| 22 | 84 | 6.2 | 3.8 | ND |
| 23 | 92 | 59 | 39 | ND |
| 32 | 98 | 15.10 | 18.10 | ND |
| 24 | 115 | 1 | 0.6 | ND |
| 30 | 121 | 15.9 | 7.9 | ND |
| 25 | 127 | 0.5 | 0.5 | 0.1 |
| 26 | 128 | 0.7 | 0.8 | ND |
| 27 | 129 | 0.7 | 0.4 | ND |
| 28 | 130 | 1.3 | 0.8 | ND |
| 31 | 140 | 14.00 | 7.20 | ND |
| 33 | 141 | 50.40 | 40.10 | ND |
| 34 | 142 | 34.20 | 34.80 | ND |
| 35 | 143 | 17.00 | 12.30 | ND |
| 36 | 144 | 9.30 | 5.30 | ND |
| 39 | 145 | 6.00 | 4.60 | ND |
| 40 | 146 | 14.10 | 10.10 | ND |
| 41 | 147 | 4.70 | 1.60 | ND |
| 42 | 148 | 4.99 | 1.88 | ND |
| 43 | 149 | 4.34 | 1.74 | ND |
| 44 | 150 | 7.50 | 3.10 | ND |
| 45 | 151 | 16.90 | 7.00 | ND |
| 46 | 152 | 14.20 | 5.70 | ND |
| 47 | 153 | 4.80 | 1.30 | ND |
| 48 | 154 | 4.30 | 1.30 | ND |
| 49 | 155 | 15.69 | 9.62 | ND |
| 50 | 156 | 3.70 | 3.20 | ND |
| 51 | 157 | 3.00 | 2.20 | ND |
| 52 | 158 | 5.90 | 2.25 | ND |
| 53 | 159 | 9.29 | 4.39 | ND |
| 54 | 160 | 38.39 | 26.49 | ND |
| 56 | 162 | 11.50 | 4.30 | ND |
| 57 | 163 | 0.60 | 0.40 | 0.26 |
| 58 | 164 | 1.10 | 0.50 | 0.26 |
| 59 | 165 | 1.10 | 0.90 | 0.41 |
| 60 | 166 | 2.80 | 1.00 | 0.14, 0.18 |
| 61 | 167 | 1.50 | 0.60 | 0.50 |
| 62 | 168 | 15.80 | 9.60 | ND |
| 63 | 169 | 2.50 | 1.30 | ND |
| 64 | 170 | 17.71 | 5.36 | ND |
| 65 | 171 | 2.52 | 1.03 | ND |
| 66 | 172 | 1.00 | 0.50 | ND |
| 67 | 173 | 0.90 | 0.50 | ND |
| 68 | 174 | 1.50 | 0.60 | ND |
| 69 | 175 | 1.20 | 0.30 | ND |
| 70 | 176 | 2.00 | 0.60 | ND |
| 71 | 177 | 1.60 | 0.60 | ND |
| 72 | 178 | 1.20 | 0.43 | ND |
| 73 | 179 | 0.80 | 0.20 | ND |

ND = Not Determined

1. A compound of Formula (I): 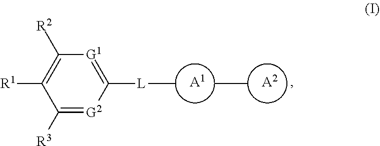
or a tautomer thereof, or a pharmaceutically acceptable salt of any of the foregoing,
wherein
R¹ is R^{1A} and R² is R^{2A}, or R¹ is R^{2A} and R² is R^{1A},
wherein R^{1A} is —OH, —OPO₃H₂, —OCH₂OPO₃H₂, —OC(O)R^{1A1}, —OC(O)OR^{1A1}, —OC(O)NHR^{1A1}, —OC(O)NR^{1A1}R^{1A2}, or —OR^{1A3},
wherein R^{1A1} and R^{1A2} are each independently hydrogen, optionally substituted alkyl, optionally substituted alkenyl, optionally substituted alkynyl, optionally substituted cycloalkyl, optionally substituted cycloalkenyl, optionally substituted aryl, optionally substituted heterocyclyl, optionally substituted heteroaryl, or —O₀₋₁(CH₂)ₘO(CH₂)ₙOH, wherein m and n are each independently 1 or 2, and
R^{1A3} is optionally substituted heteroaryl;
R^{2A} is —CHO or —CH═NR^{2A1},
wherein R^{2A1} is optionally substituted heterocyclyl, optionally substituted alkyl, optionally substituted alkenyl, optionally substituted alkynyl, —NR^{2A1A}C(O)R^{2A1B}, —NR^{2A1A}S(O)₂R^{2A1B}, —NR^{2A1A}R^{2A1B}, —OR^{2A1A} or —NR^{2A1A}C(NR^{2A1B})NR^{2A1}CR^{2A1D}, and
wherein R^{2A1A}, R^{2A1B}, R^{2A1C}, and R^{2A1D} are each independently hydrogen, optionally substituted alkyl, optionally substituted cycloalkyl, optionally substituted heteroaryl, optionally substituted aryl, or optionally substituted amino; or
R^{1A} and R^{2A} taken together with 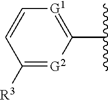
to which they are attached form optionally substituted 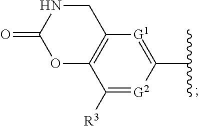
R³ is halo, hydrogen, optionally substituted alkyl, or optionally substituted alkoxy;
G¹ and G² are each independently CH or N;
L is a bond, —C(O)NH—*, —NHC(O)—*, —C(R^{4A})(R^{4B})NHC(O)—*, —C(O)—, —S(O)₂—, —S(O)₂NH—*, 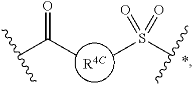
—C(O)N(R^{4D})(CH₂)₂₋₃—*, —C(O)N(CH₃)—*, —(CH₂)OC(O)NH—*, —C(O)NHNH—*, —C(O)NHNHC(O)—*, —CH(R^{4E})NHC(O)O—*, or —C(O)NHO—*,
wherein R^{4A}, R^{4B}, R^{4D}, and R^{4E} are each independently hydrogen or optionally substituted alkyl,
R^{4C} is optionally substituted aryl, optionally substituted cycloalkyl, optionally substituted cycloalkenyl, optionally substituted heteroaryl, or optionally substituted heterocyclyl, and
represents the point of attachment to A¹; and
A¹ and A² are each independently optionally substituted aryl, optionally substituted cycloalkyl, optionally substituted cycloalkenyl, optionally substituted heteroaryl, or optionally substituted heterocyclyl;
provided that
when R¹ is —OH, R³ is fluoro, L is a bond, and A¹ is optionally substituted 5-membered heteroaryl, then A² is not optionally substituted phenyl, optionally substituted pyridinyl, optionally substituted pyrazinyl, or 2,3-dihydrobenzo[b][1,4]dioxin-6-yl;
when R¹ is —CHO, R² is —OH, R³ is hydrogen, and L is —C(O), then A¹ is not optionally substituted indolinyl;
when L is —C(O)NH—*, then A¹ is not optionally substituted phenyl, optionally substituted pyridinyl, or pyrimidinyl;
when R³ is hydrogen, C₁₋₄ alkyl, —CHO, or methoxy, then L is not a bond; and
the compound of Formula (I) is not 3-fluoro-5-formyl-4-hydroxy-N-(4-(pyrrolidin-1-yl)phenyl)benzenesulfonamide, 5-(4-(5-fluoropyridin-2-yl)piperazine-1-carbonyl)-2-hydroxy-3-methylbenzaldehyde, 5-(3-(1λ⁴,2λ²,4-triazol-1-yl)azetidine-1-carbonyl)-2-hydroxy-3-methylbenzaldehyde, tert-butyl (3-(1-(3-formyl-4-hydroxybenzoyl)piperidin-4-yl)benzyl)carbamate, 5-(4-cyclopropyl-3-oxopiperazine-1-carbonyl)-2-hydroxy-3-methylbenzaldehyde, 2-(5-((((4-formyl-3-hydroxybenzyl)oxy)carbonyl)amino)benzo[d]thiazol-2-yl)-4,5-dihydrothiazole-4-carboxylic acid, 4-formyl-3-hydroxybenzyl (6-(benzo[d]oxazol-2-yl)naphthalen-2-yl)carbamate, 5-[2-(3,4-diethoxyphenyl)-4-thiazolyl]-3-formyl-2-hydroxy-benzoic acid methyl ester, or a salt of any of the foregoing.

2. The compound of claim 1, or a tautomer thereof, or a pharmaceutically acceptable salt of any of the foregoing, wherein R¹ is —OH.

3. The compound of claim 1, or a tautomer thereof, or a pharmaceutically acceptable salt of any of the foregoing, wherein R² is —CHO.

4. (canceled)

5. The compound of claim 1, or a tautomer thereof, or a pharmaceutically acceptable salt of any of the foregoing, wherein R³ is fluoro.

6. The compound of claim 1, or a tautomer thereof, or a pharmaceutically acceptable salt of any of the foregoing, wherein R³ is hydrogen, methyl, or methoxy.

7-8. (canceled)

9. The compound of claim 1, or a tautomer thereof, or a pharmaceutically acceptable salt of any of the foregoing, wherein G¹ and G² are both CH.

10. The compound of claim 1, or a tautomer thereof, or a pharmaceutically acceptable salt of any of the foregoing, wherein G¹ is CH and G² is N.

11. The compound of claim 1, or a tautomer thereof, or a pharmaceutically acceptable salt of any of the foregoing, wherein L is a bond, —C(O)NH—*, or —NHC(O)—*.

12-13. (canceled)

14. The compound of claim 1, or a tautomer thereof, or a pharmaceutically acceptable salt of any of the foregoing, wherein L is —C(O)—.

15. The compound of claim 1, or a tautomer thereof, or a pharmaceutically acceptable salt of any of the foregoing, wherein A¹ is optionally substituted aryl, optionally substituted cycloalkyl, or optionally substituted heteroaryl.

16-17. (canceled)

18. The compound of claim 1, or a tautomer thereof, or a pharmaceutically acceptable salt of any of the foregoing, wherein A¹ is optionally substituted heterocyclyl.

19. The compound of claim 1, or a tautomer thereof, or a pharmaceutically acceptable salt of any of the foregoing, wherein A¹ is 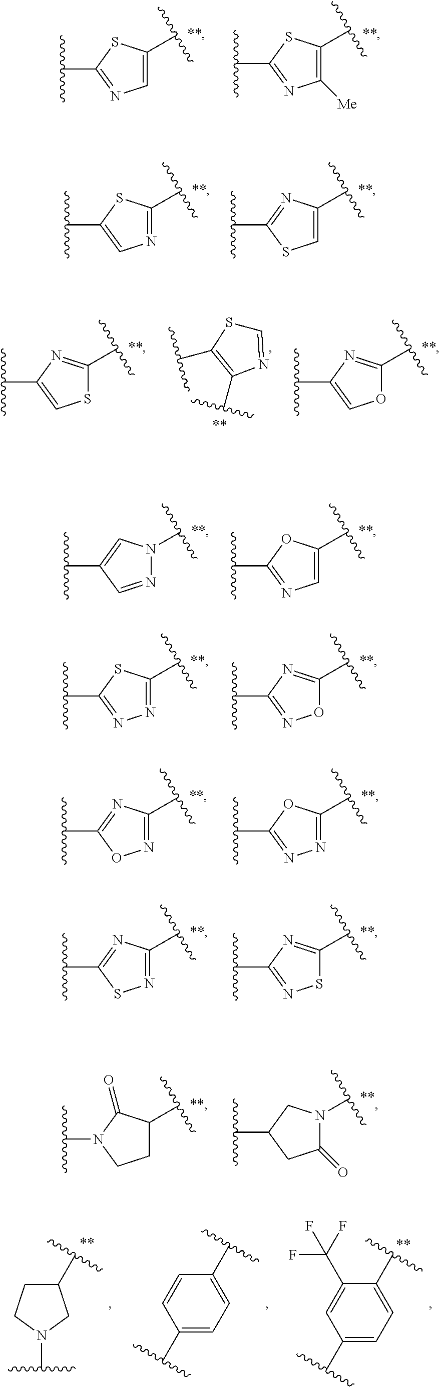 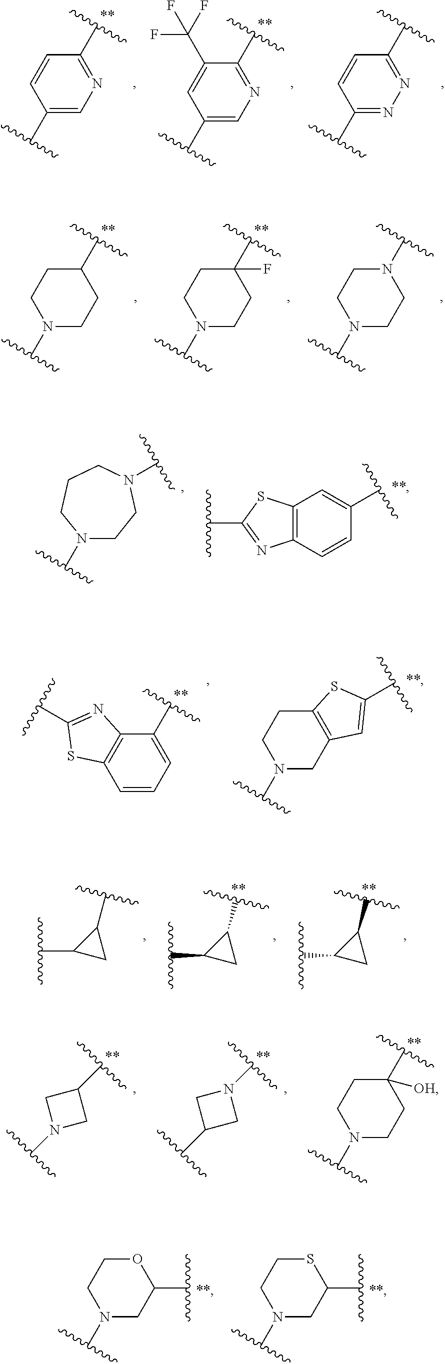 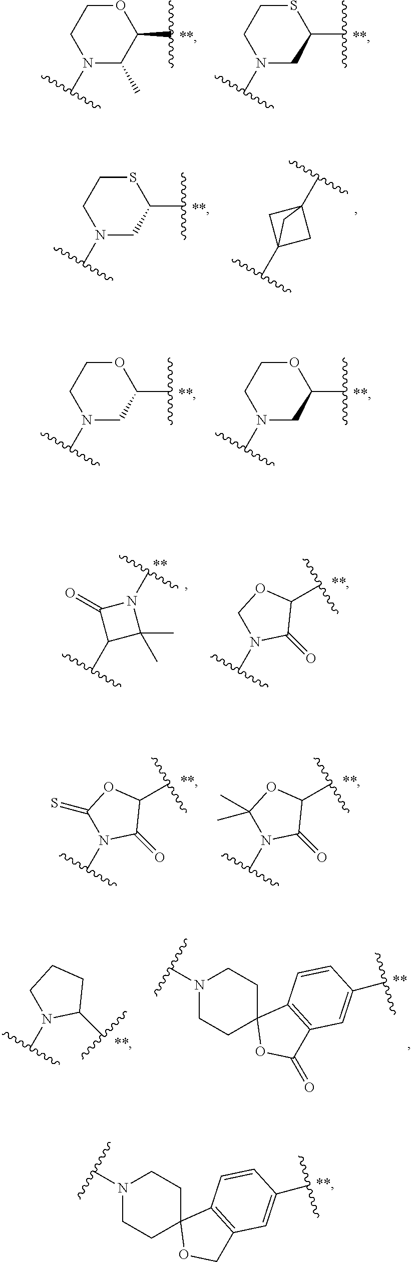 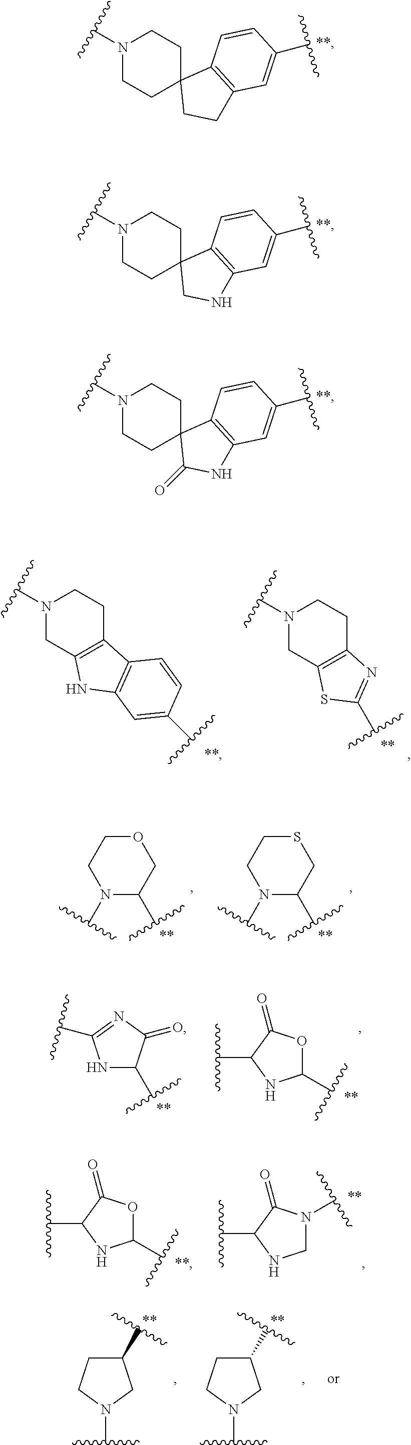 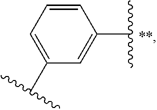 [SMILES: *c1cccc(*)c1]
wherein ** represents the point of attachment to A².

20. The compound of claim 1, or a tautomer thereof, or a pharmaceutically acceptable salt of any of the foregoing, wherein A¹ is thiadiazolyl, thiazolyl, oxazolyl, pyrazolyl, phenyl, pyridinyl, pyridazinyl, azetidinyl, pyrrolidinyl, piperidinyl, or piperazinyl, each of which is optionally substituted.

21. (canceled)

22. The compound of claim 1, or a tautomer thereof, or a pharmaceutically acceptable salt of any of the foregoing, wherein A¹ is optionally substituted 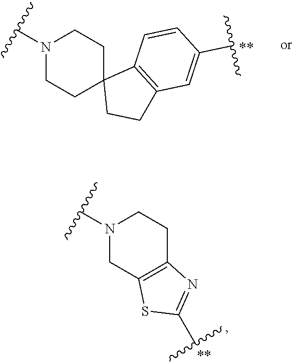 [SMILES: *c1ccc2c(c1)CCC21CCN(*)CC1 ; *c1nc2c(s1)CN(*)CC2]
wherein ** represents the point of attachment to A².

23. (canceled)

24. The compound of claim 1, or a tautomer thereof, or a pharmaceutically acceptable salt of any of the foregoing, wherein A¹ is optionally substituted piperidinyl.

25. The compound of claim 1, or a tautomer thereof, or a pharmaceutically acceptable salt of any of the foregoing, wherein A² is optionally substituted aryl.

26-27. (canceled)

28. The compound of claim 1, or a tautomer thereof, or a pharmaceutically acceptable salt of any of the foregoing, wherein A² is optionally substituted heterocyclyl.

29. The compound of claim 1, or a tautomer thereof, or a pharmaceutically acceptable salt of any of the foregoing, wherein A² is

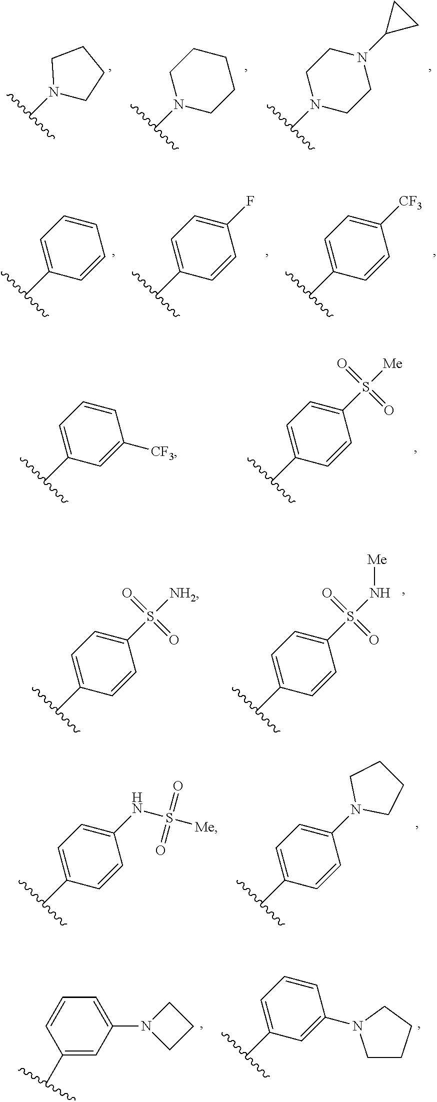

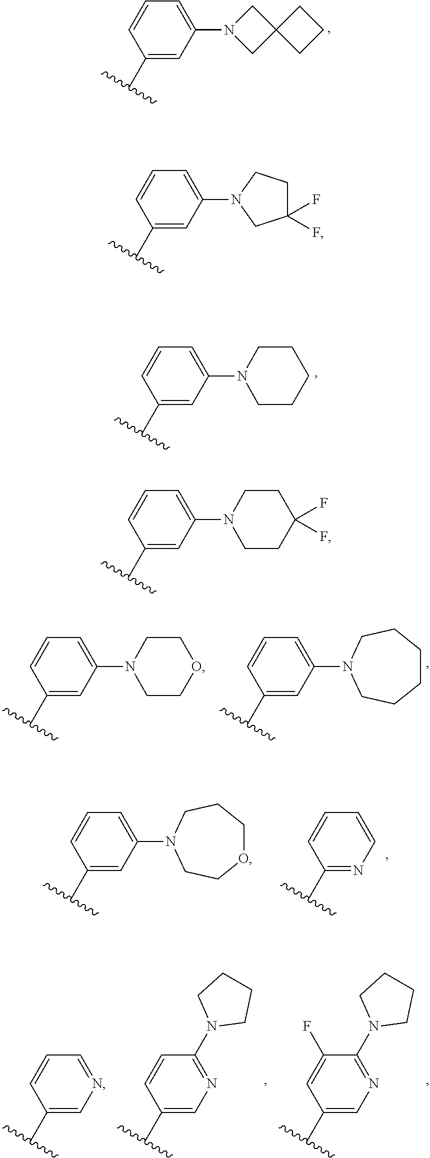

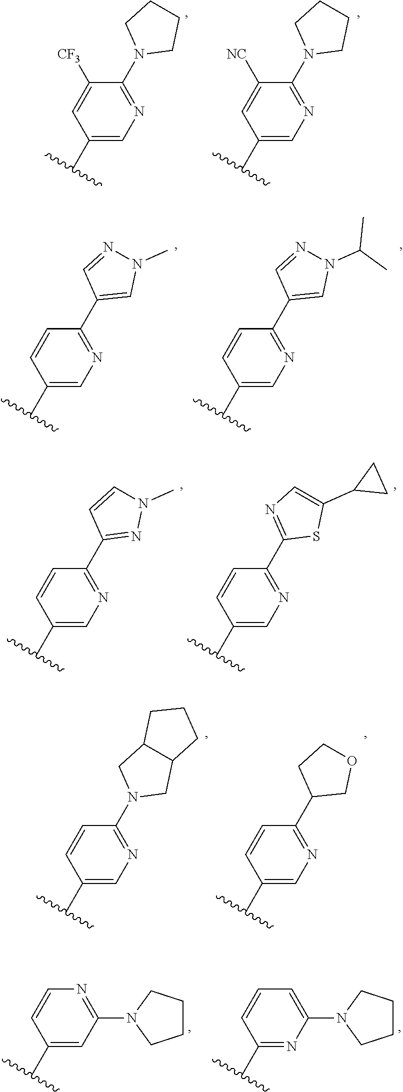

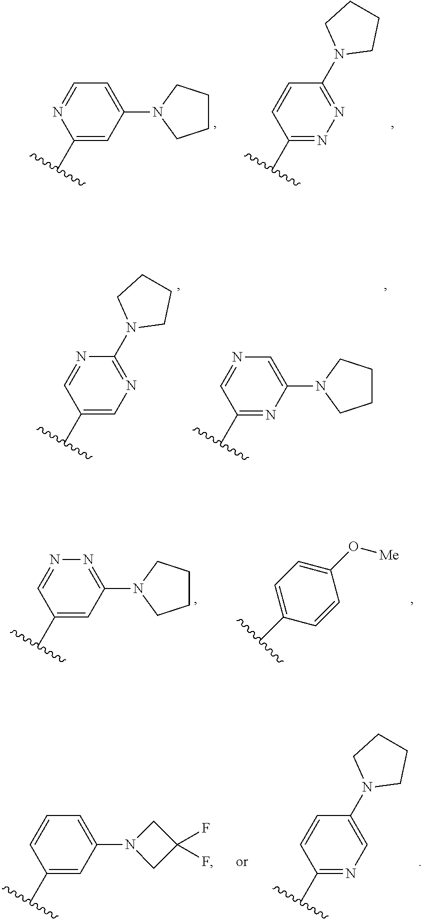

30. The compound of claim 1, or a tautomer thereof, or a pharmaceutically acceptable salt of any of the foregoing, wherein A² is pyrrolidinyl, piperidinyl, piperazinyl, phenyl, pyridinyl, pyridazinyl, pyrimidinyl, or pyrazinyl, each of which is optionally substituted.

31. The compound of claim 1, or a tautomer thereof, or a pharmaceutically acceptable salt of any of the foregoing, wherein A² is optionally substituted phenyl.

32-33. (canceled)

34. The compound of claim 1, or a tautomer thereof, or a pharmaceutically acceptable salt of any of the foregoing, wherein

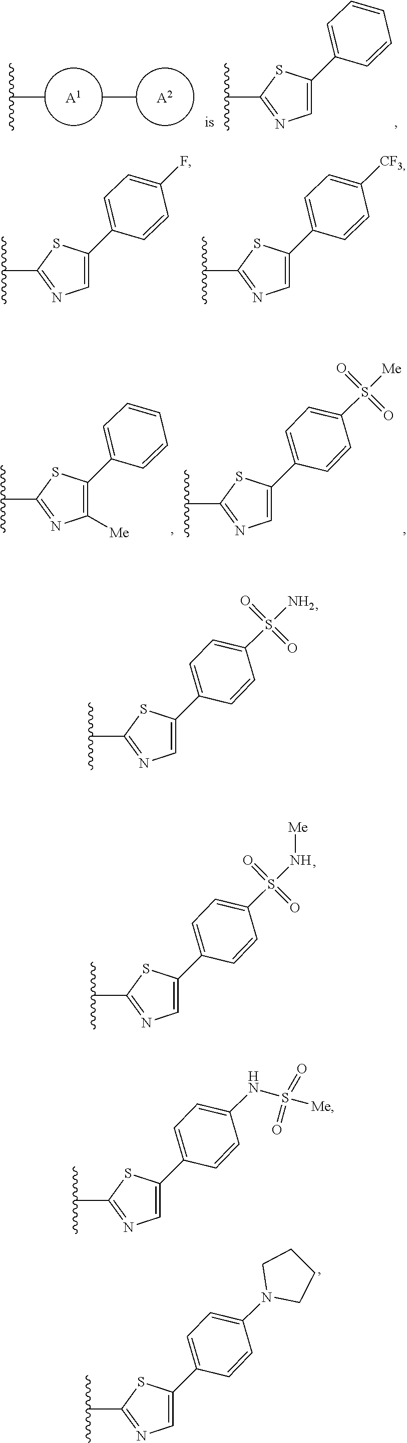

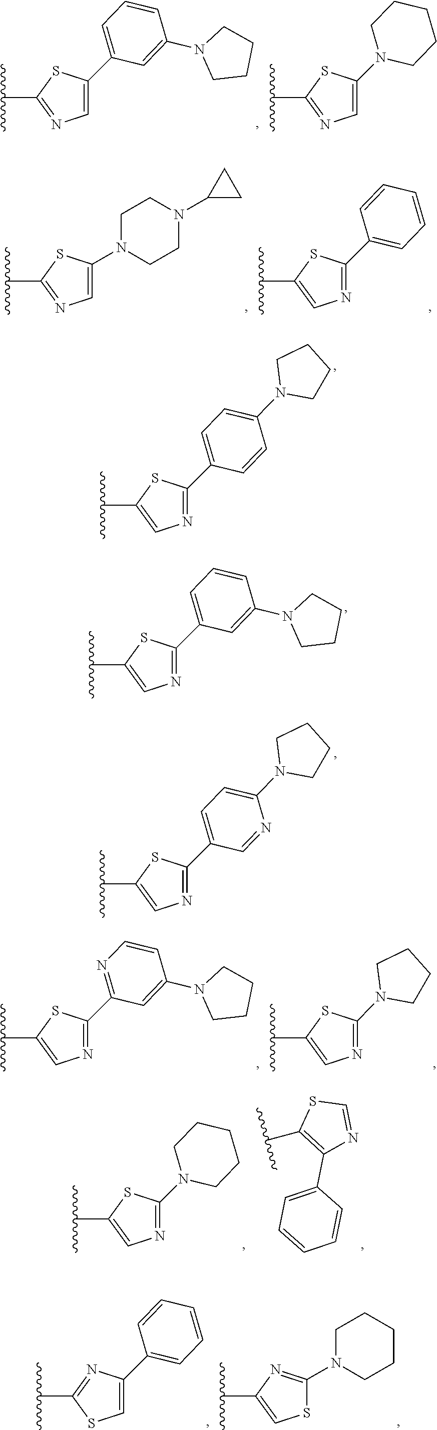

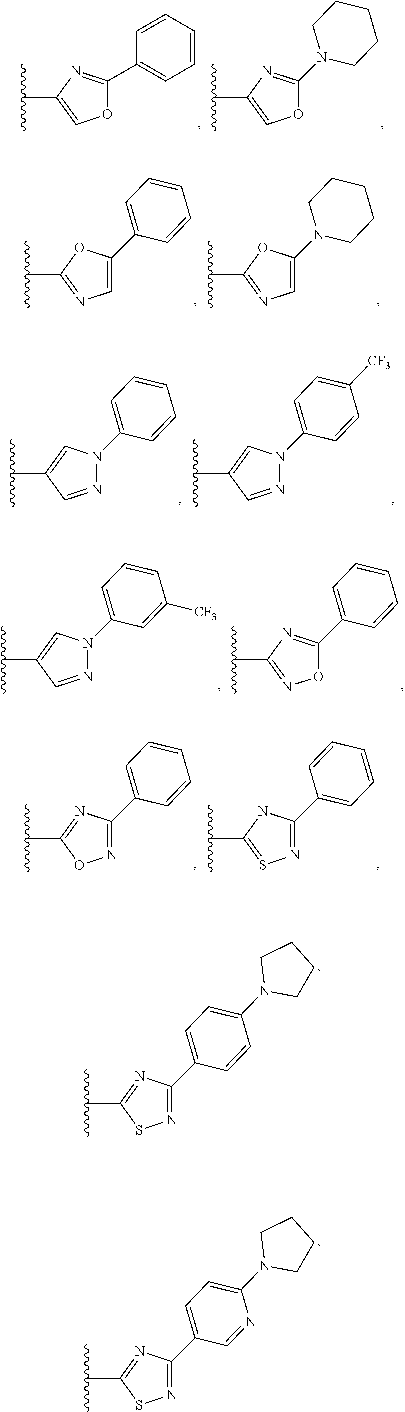

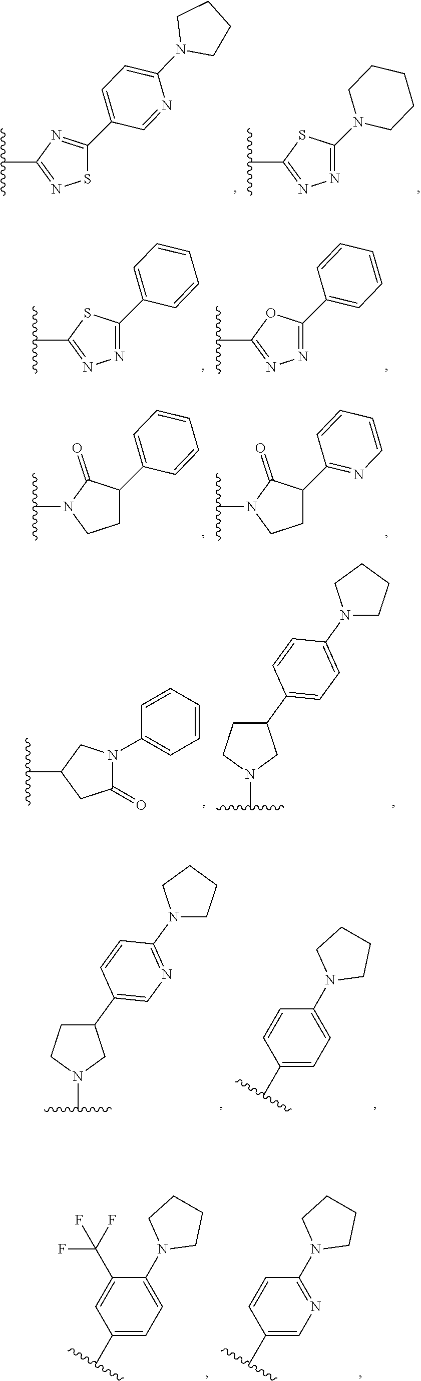

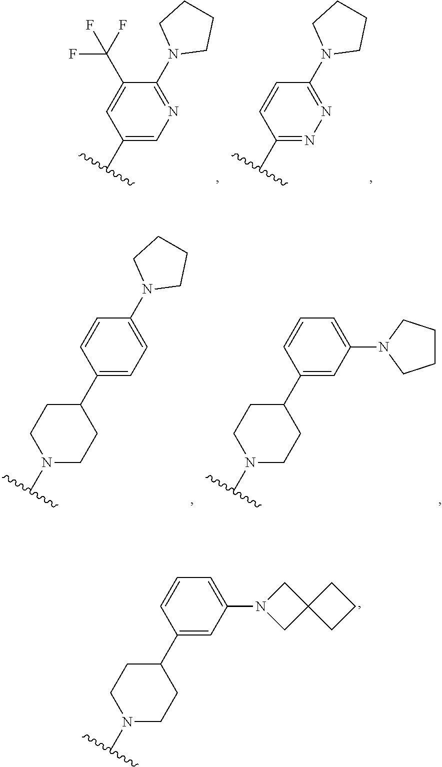 [SMILES: *c1cnc(N2CCCC2)c(C(F)(F)F)c1 ; *c1ccc(N2CCCC2)nn1 ; *N1CCC(c2ccc(N3CCCC3)cc2)CC1 ; *N1CCC(c2cccc(N3CCCC3)c2)CC1 ; *N1CCC(c2cccc(N3CC4(CCC4)C3)c2)CC1]

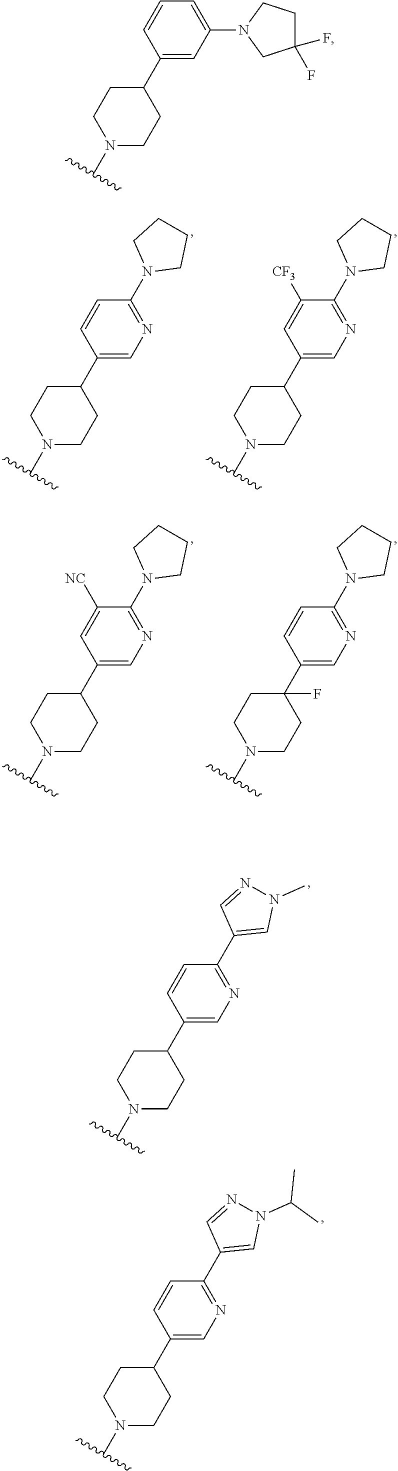

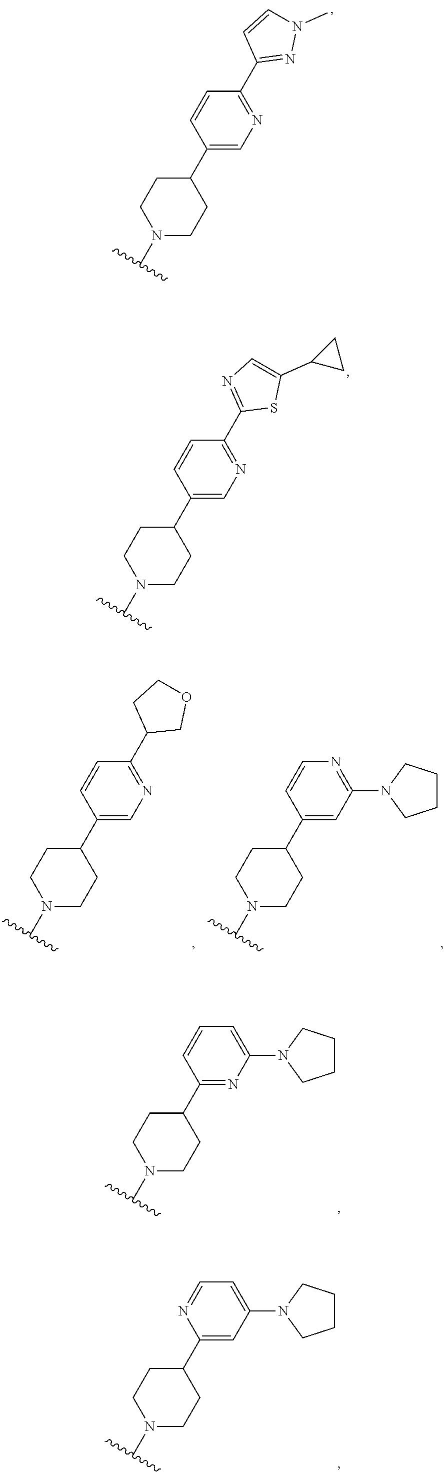 [SMILES: *N1CCC(c2ccc(-c3ccn(C)n3)nc2)CC1 ; *N1CCC(c2ccc(-c3ncc(C4CC4)s3)nc2)CC1 ; *N1CCC(c2ccc(C3CCOC3)nc2)CC1 ; *N1CCC(c2ccnc(N3CCCC3)c2)CC1 ; *N1CCC(c2cccc(N3CCCC3)n2)CC1 ; *N1CCC(c2cc(N3CCCC3)ccn2)CC1]

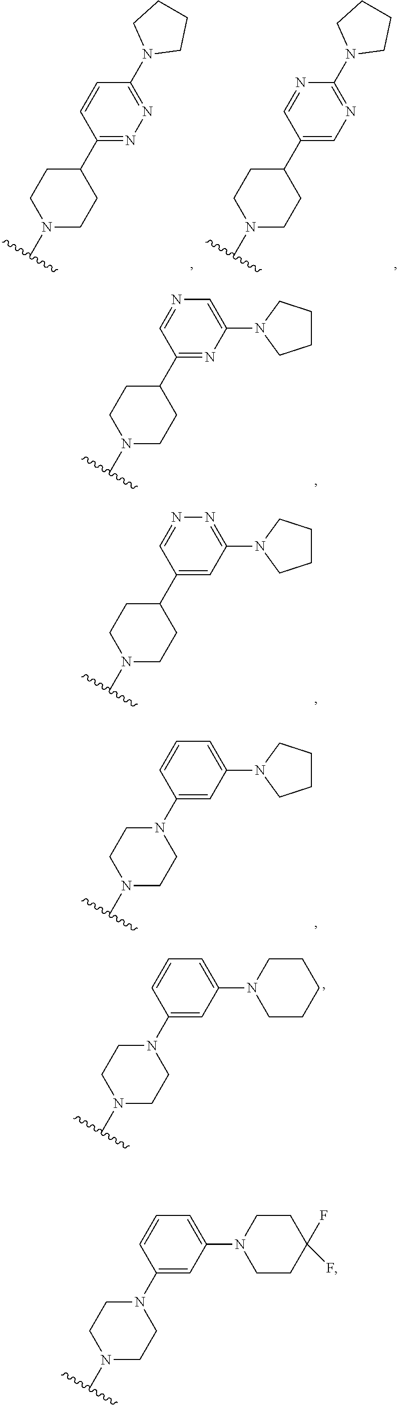

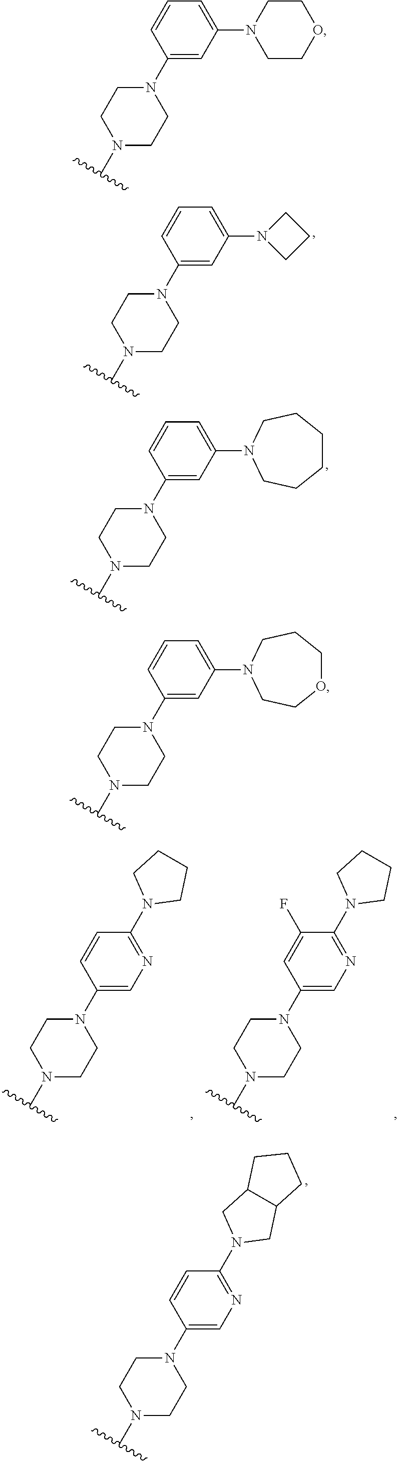

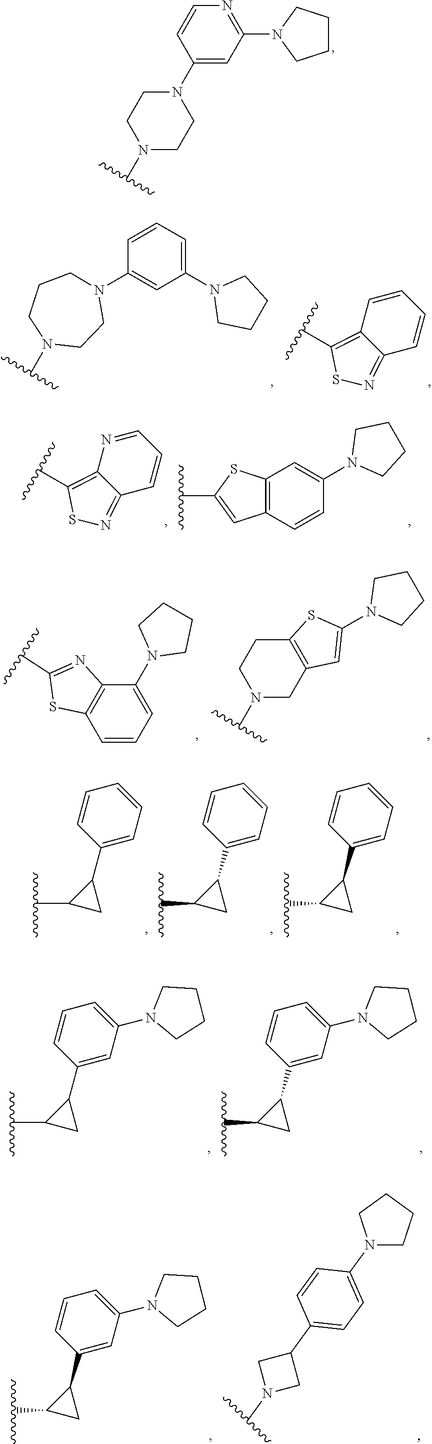

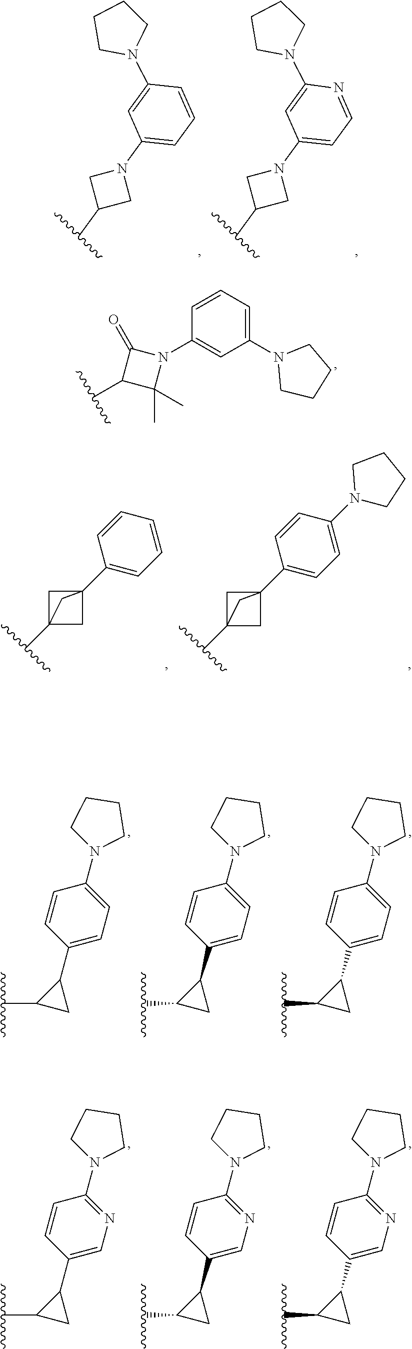

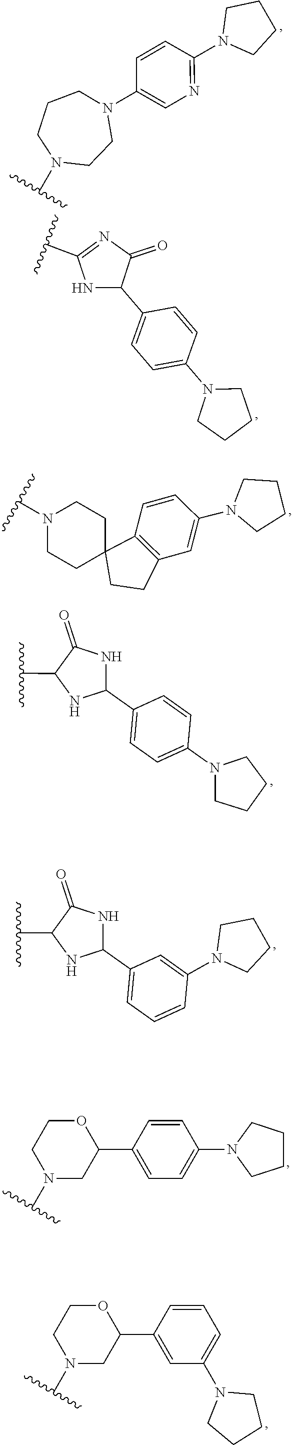

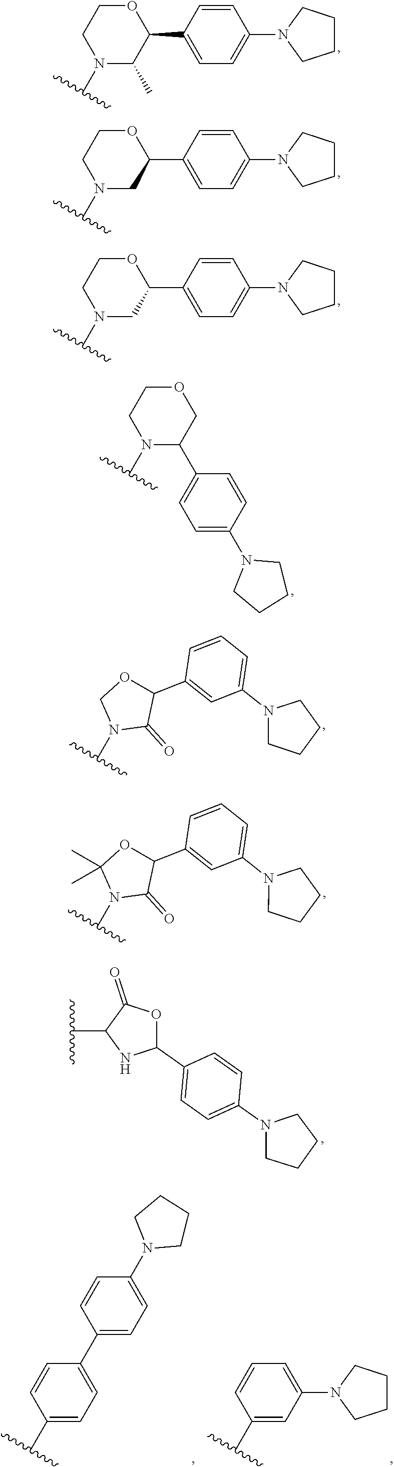

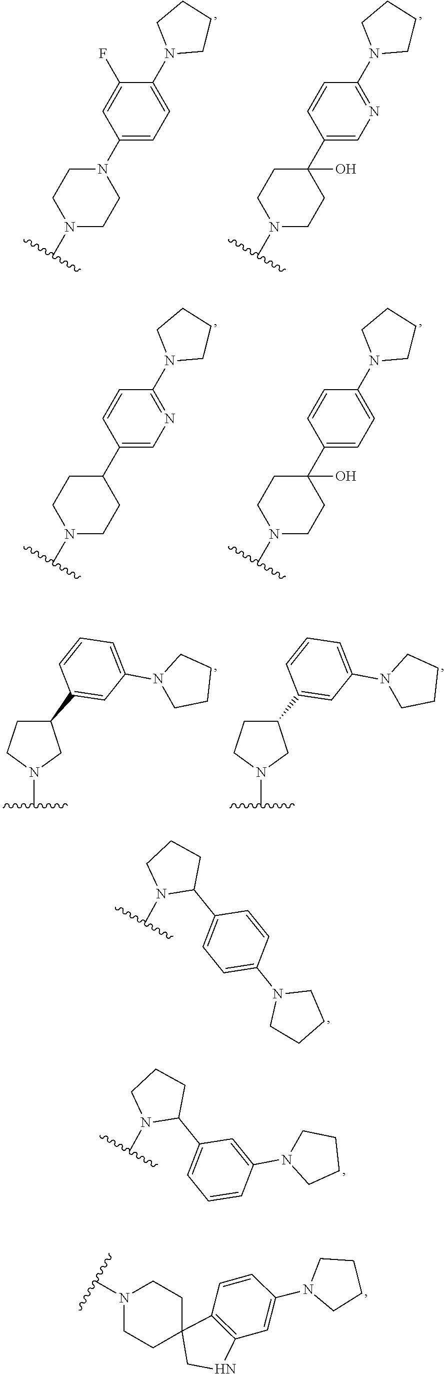

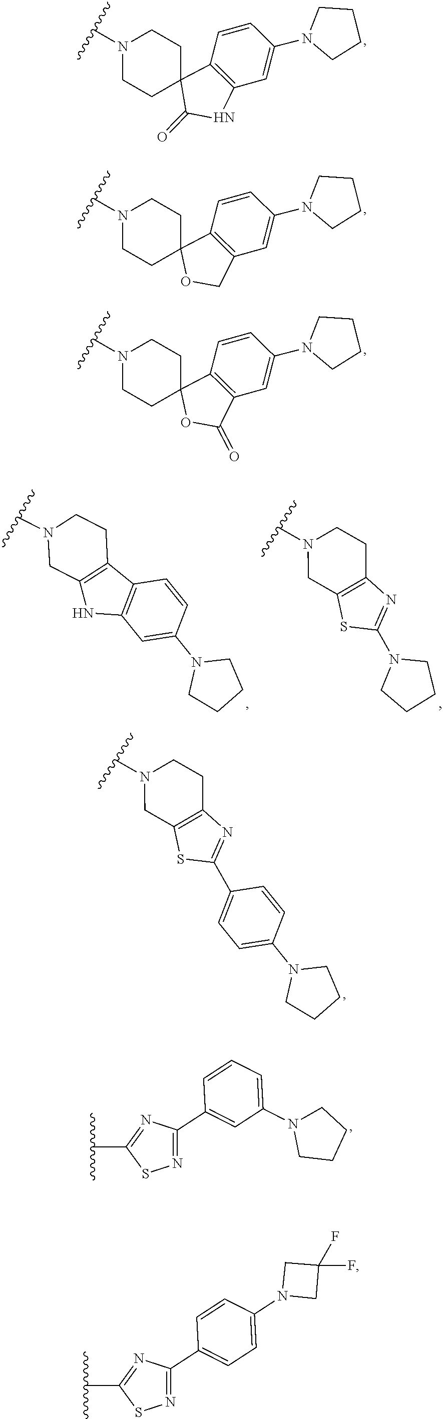

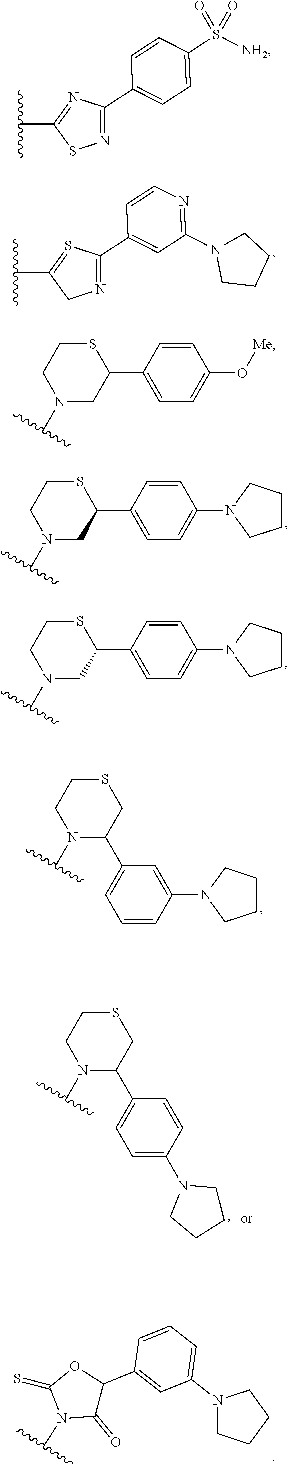

35. (canceled)

36. The compound of claim 1, or a tautomer thereof, or a pharmaceutically acceptable salt of any of the foregoing, wherein

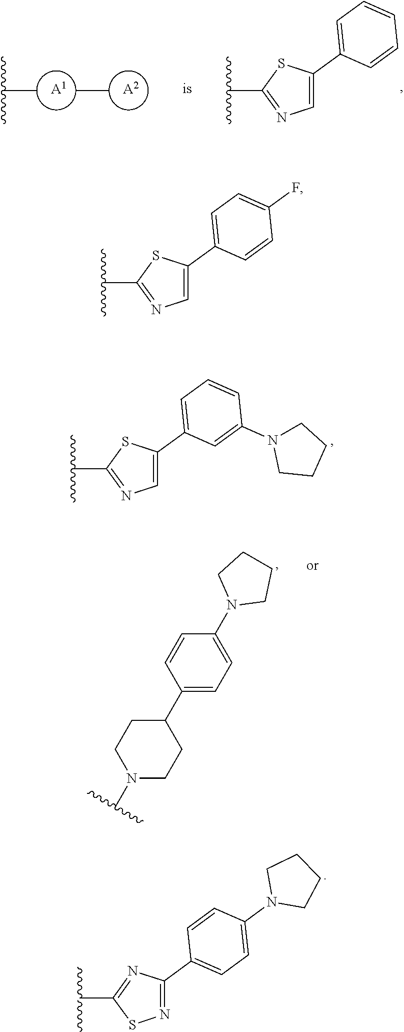

37. The compound of claim 1, or a tautomer thereof, or a pharmaceutically acceptable salt of any of the foregoing, wherein the 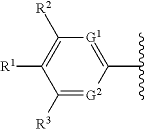
portion of the compound is

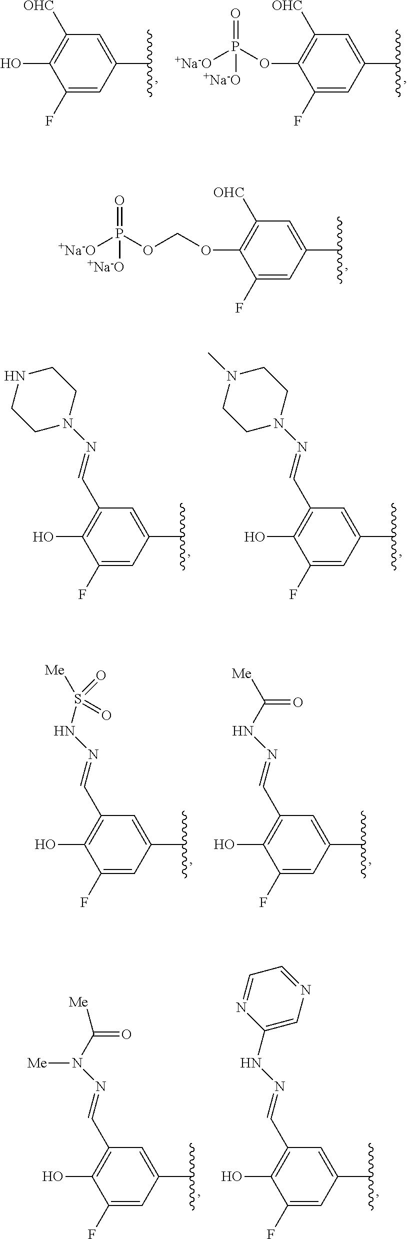

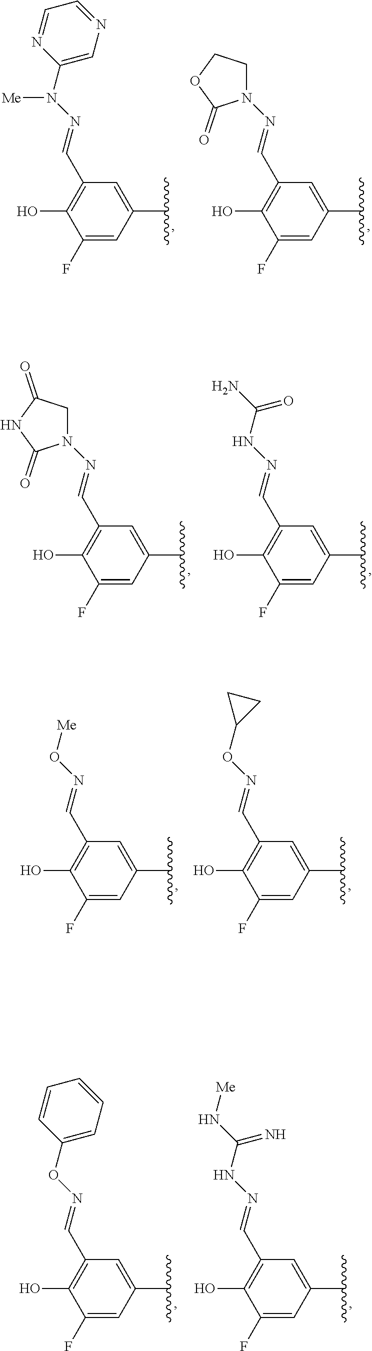

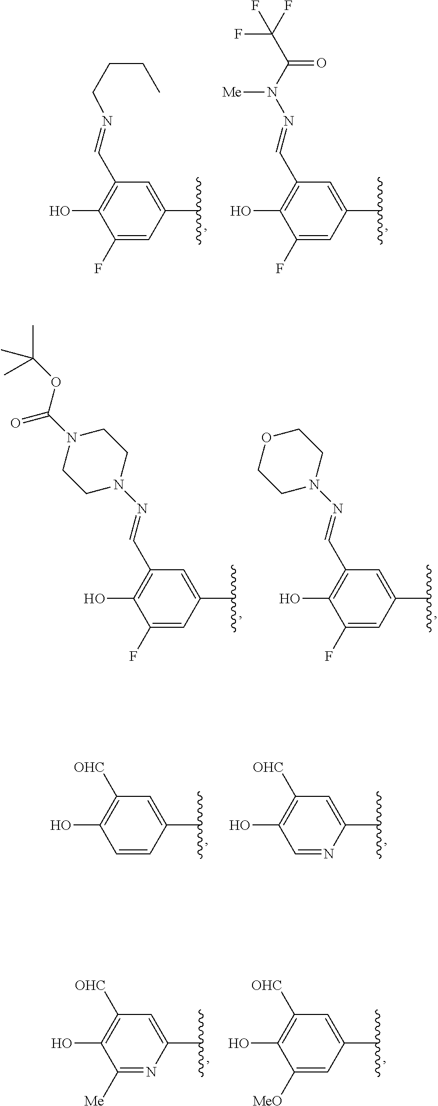

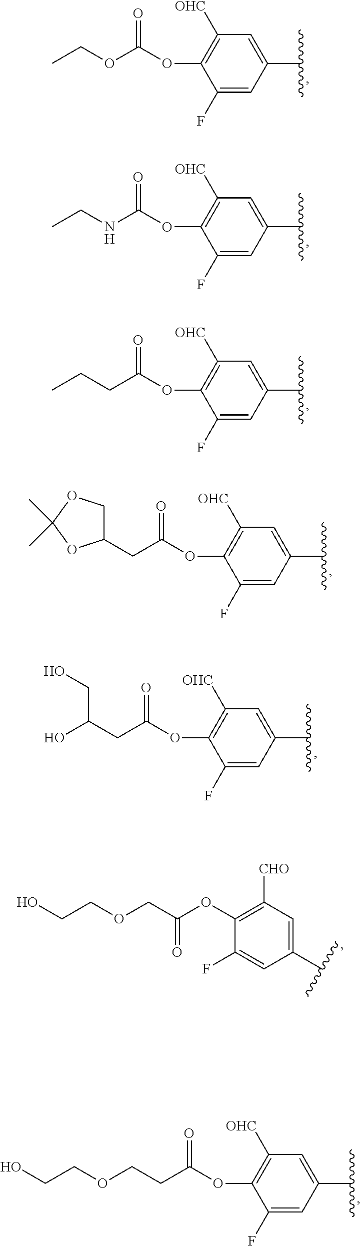

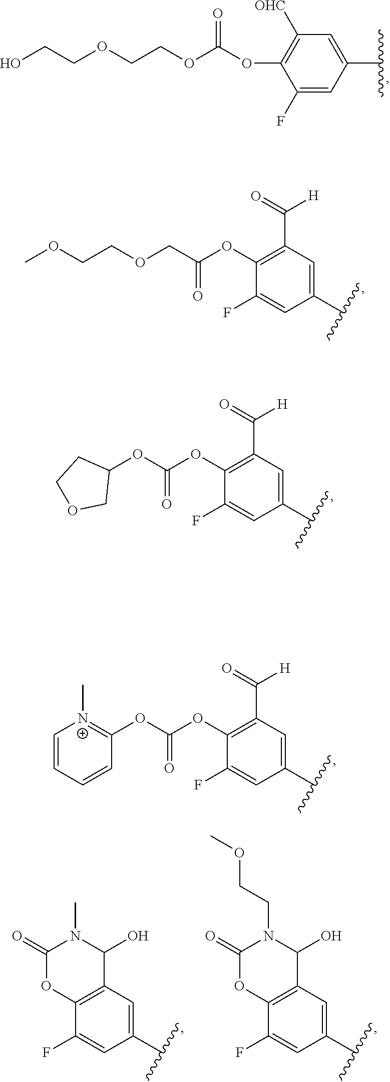 [SMILES: *c1cc(F)c(OC(=O)OCCOCCO)c(C=O)c1 ; *c1cc(F)c(OC(=O)COCCOC)c(C=O)c1 ; *c1cc(F)c(OC(=O)OC2CCOC2)c(C=O)c1 ; *c1cc(F)c(OC(=O)Oc2cccc[n+]2C)c(C=O)c1 ; *c1cc(F)c2c(c1)C(O)N(C)C(=O)O2 ; *c1cc(F)c2c(c1)C(O)N(CCOC)C(=O)O2]

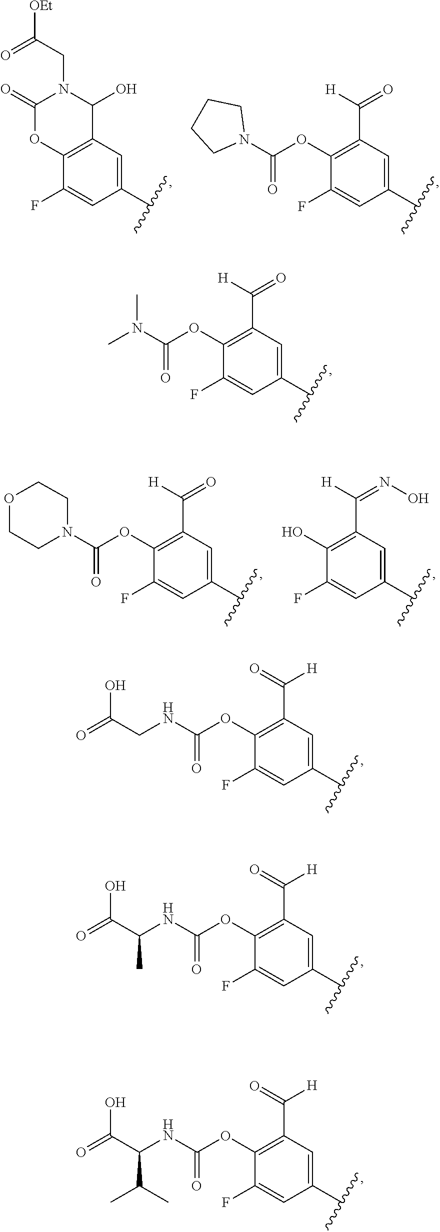

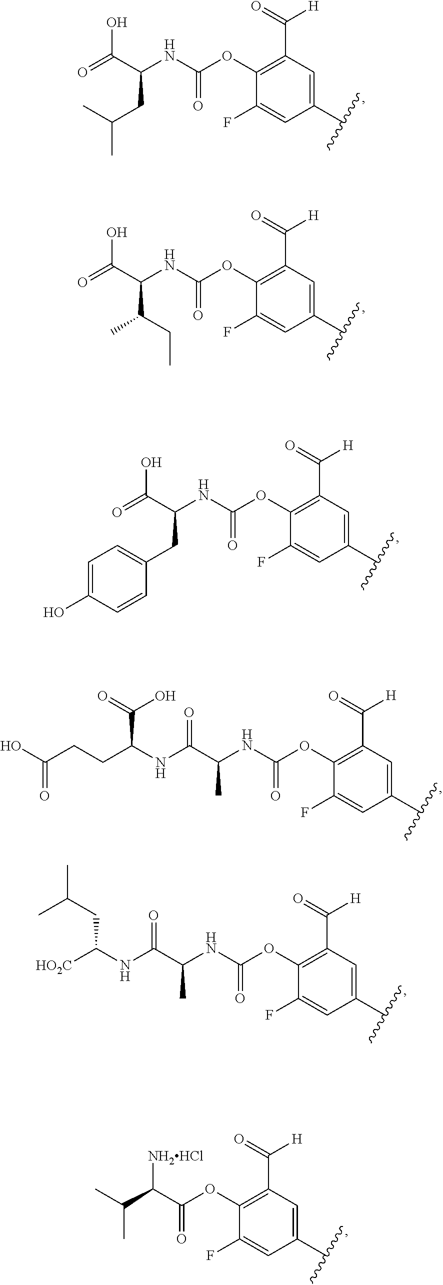

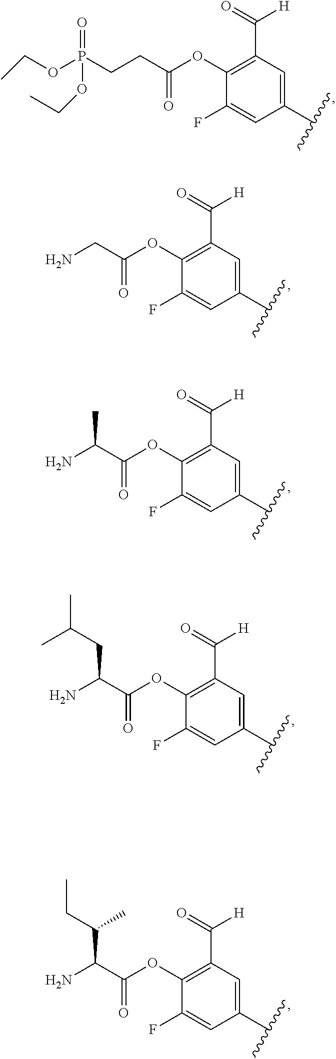 [SMILES: *c1cc(F)c(OC(=O)CCP(=O)(OCC)OCC)c(C=O)c1 ; *c1cc(F)c(OC(=O)CN)c(C=O)c1 ; *c1cc(F)c(OC(=O)[C@H](C)N)c(C=O)c1 ; *c1cc(F)c(OC(=O)[C@@H](N)CC(C)C)c(C=O)c1 ; *c1cc(F)c(OC(=O)[C@@H](N)[C@@H](C)CC)c(C=O)c1]

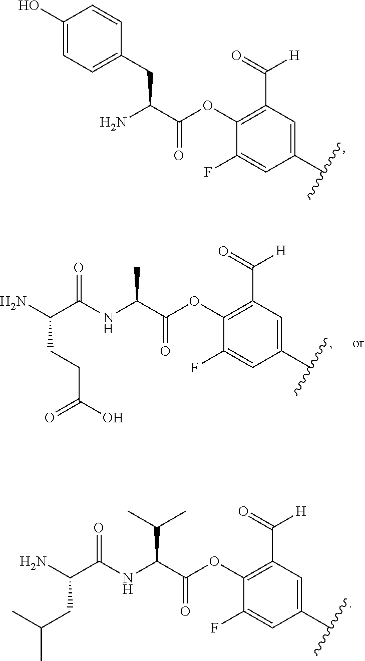 [SMILES: *c1cc(F)c(OC(=O)[C@@H](N)Cc2ccc(O)cc2)c(C=O)c1 ; *c1cc(F)c(OC(=O)[C@H](C)NC(=O)[C@@H](N)CCC(=O)O)c(C=O)c1 ; *c1cc(F)c(OC(=O)[C@@H](NC(=O)[C@@H](N)CC(C)C)C(C)C)c(C=O)c1]

38. The compound of claim 1, or a tautomer thereof, or a pharmaceutically acceptable salt of any of the foregoing, wherein the 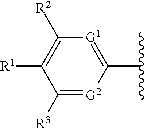
portion of the compound is

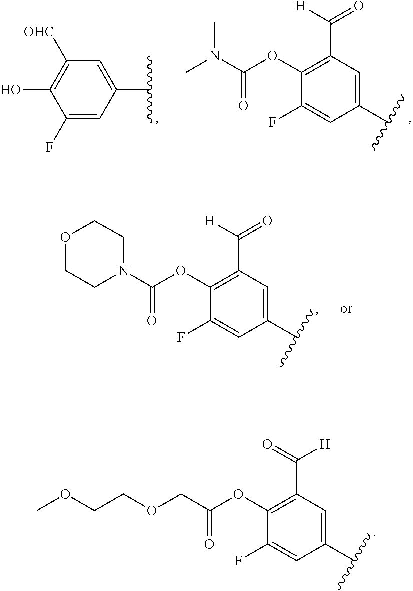

39. A compound, or a tautomer thereof, or a pharmaceutically acceptable salt of any of the foregoing, wherein the compound is selected from the group consisting of

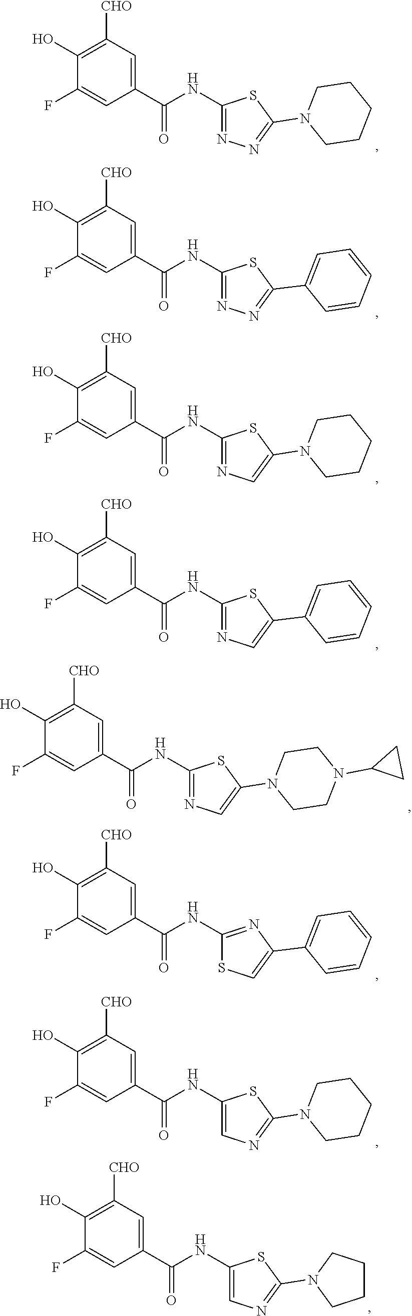

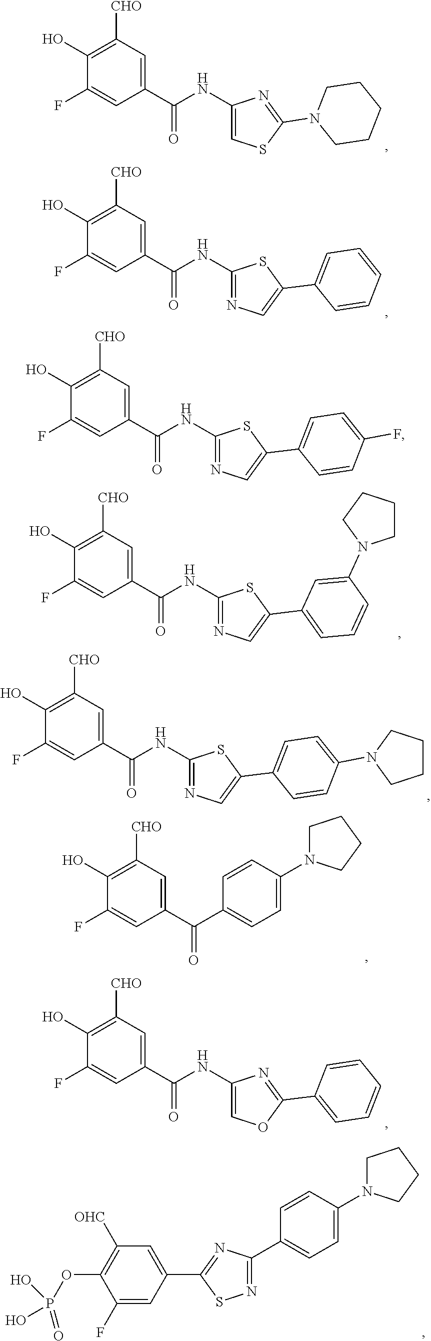

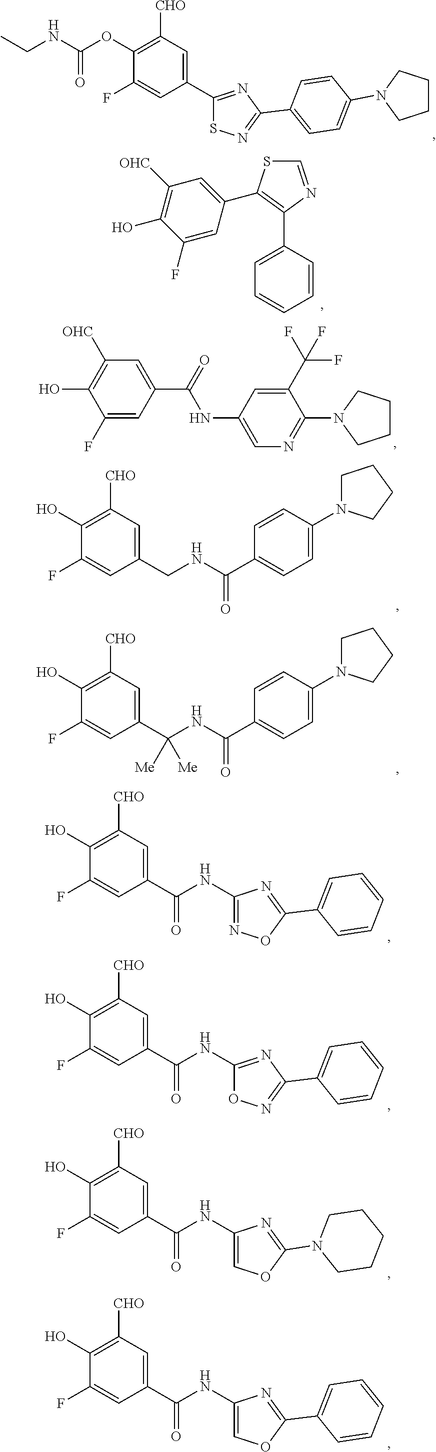

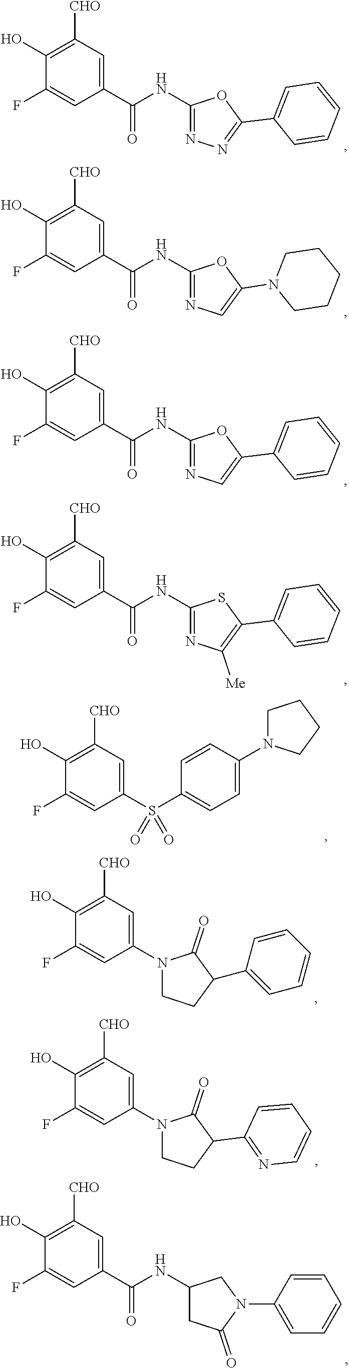

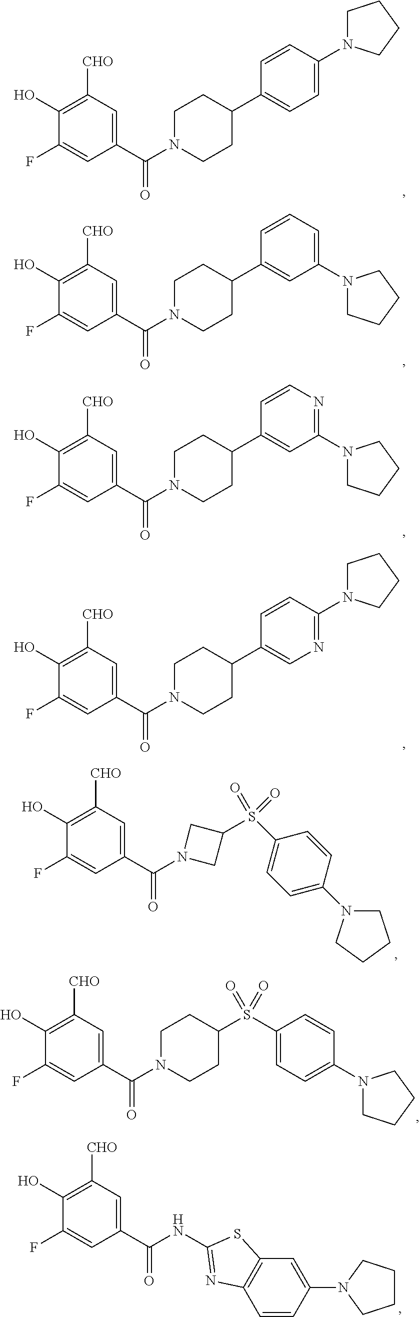

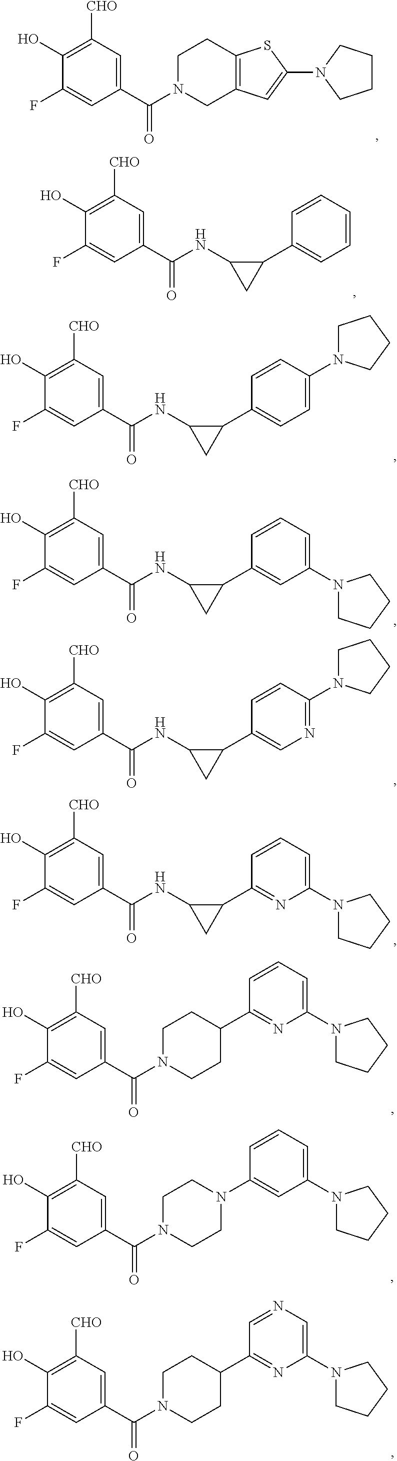

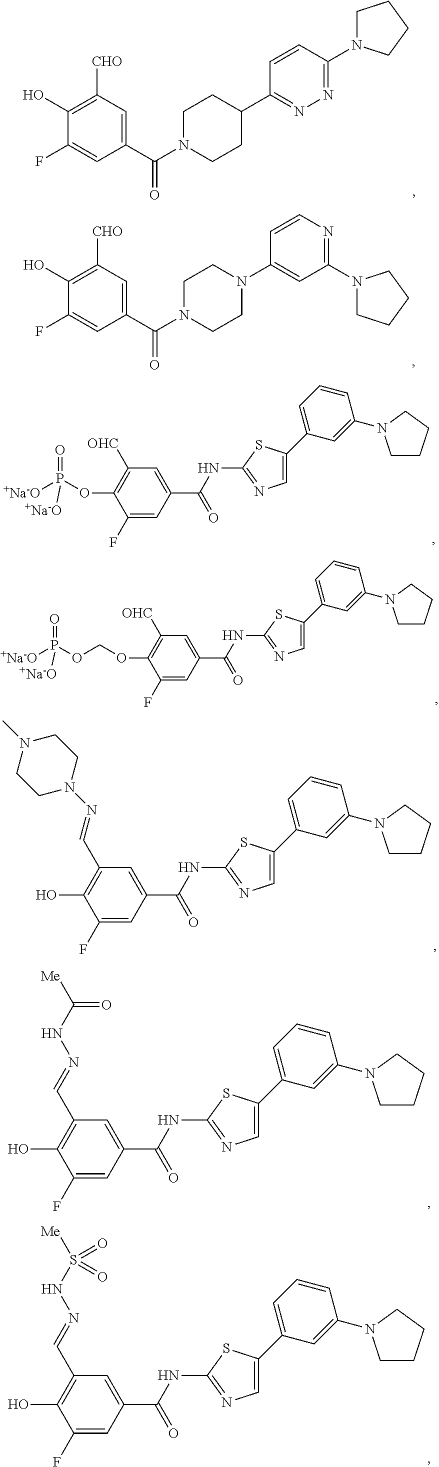

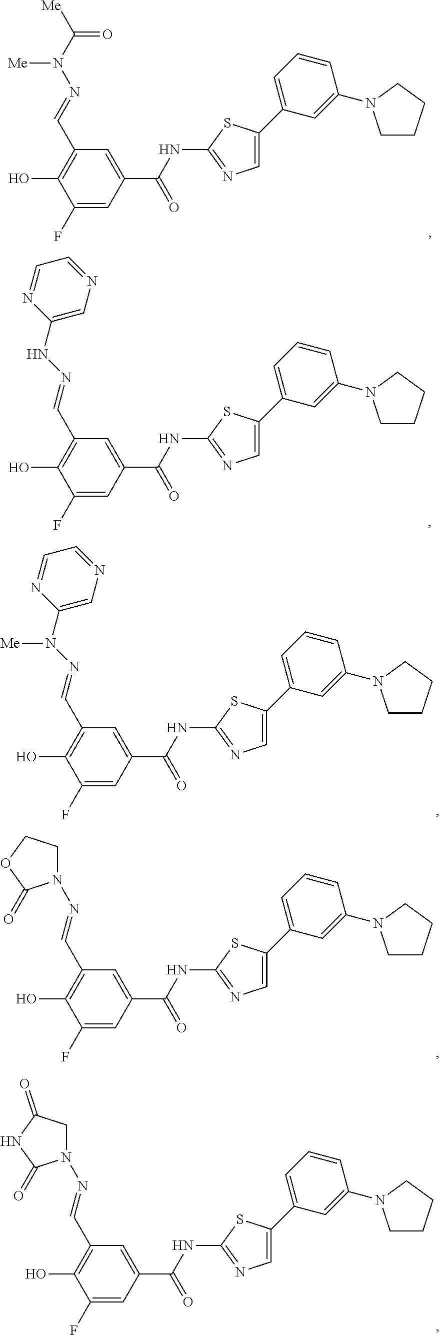

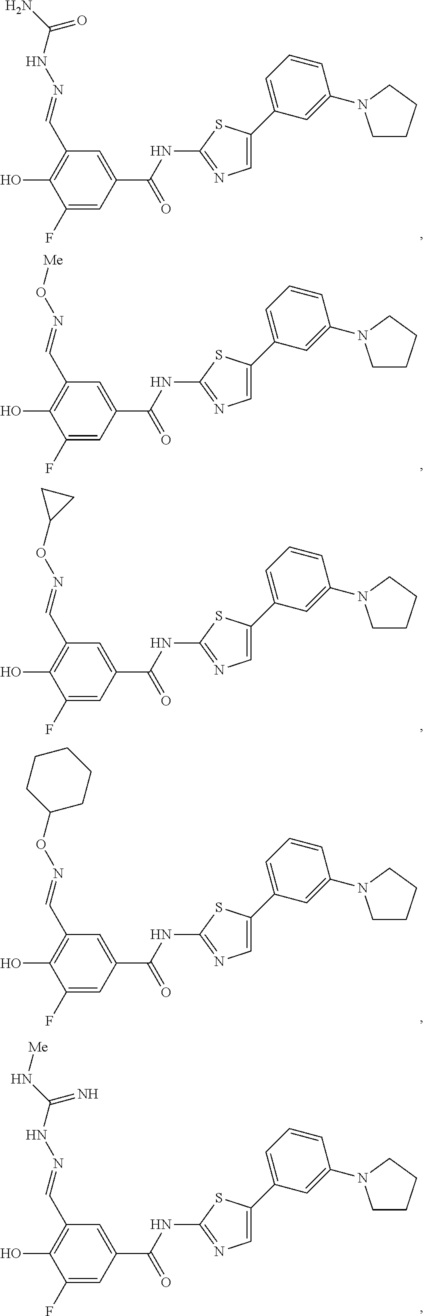

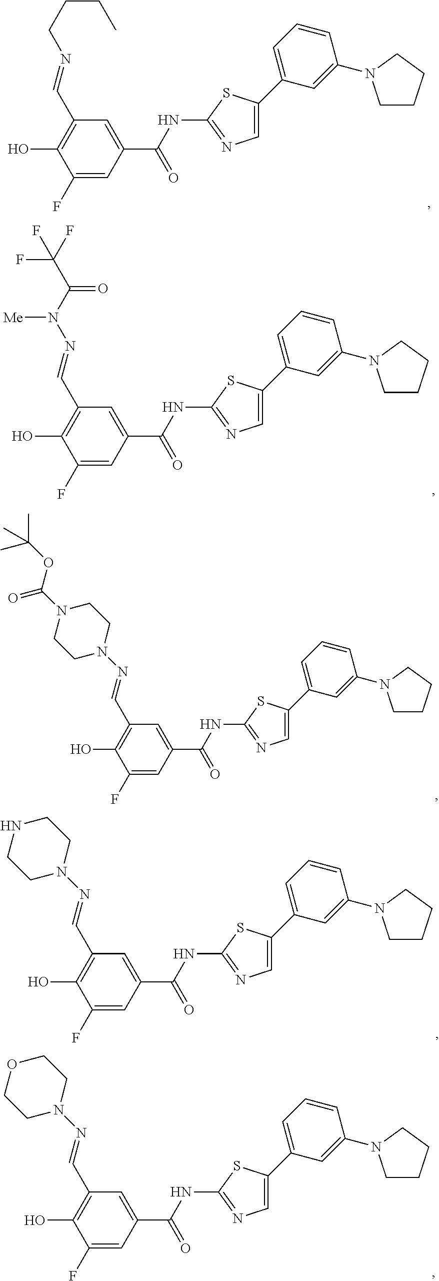

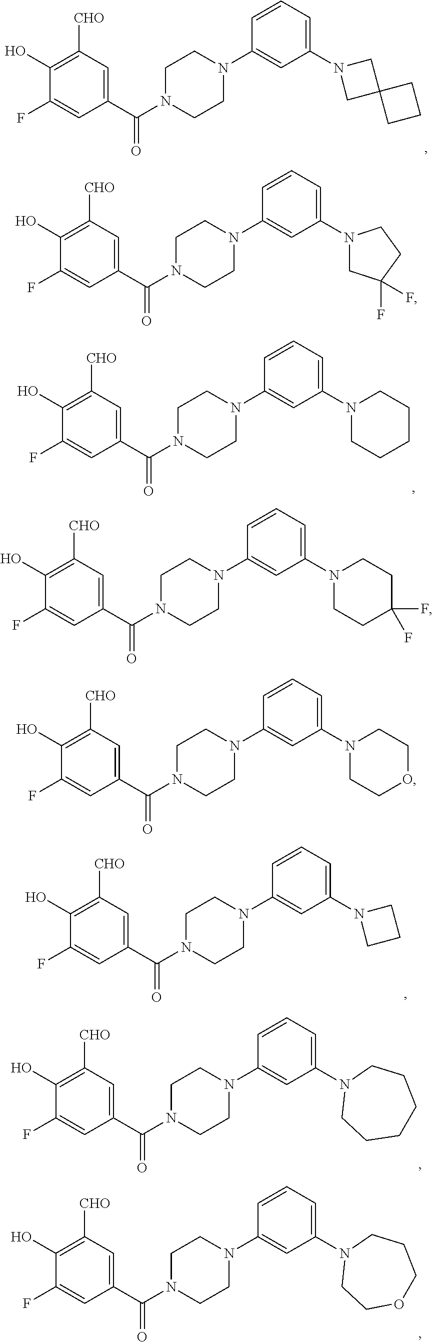

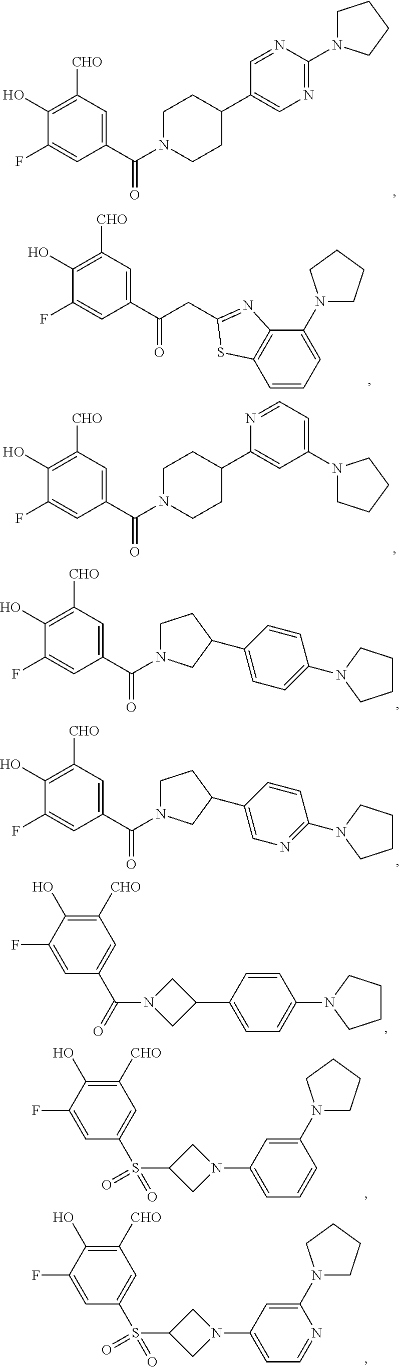

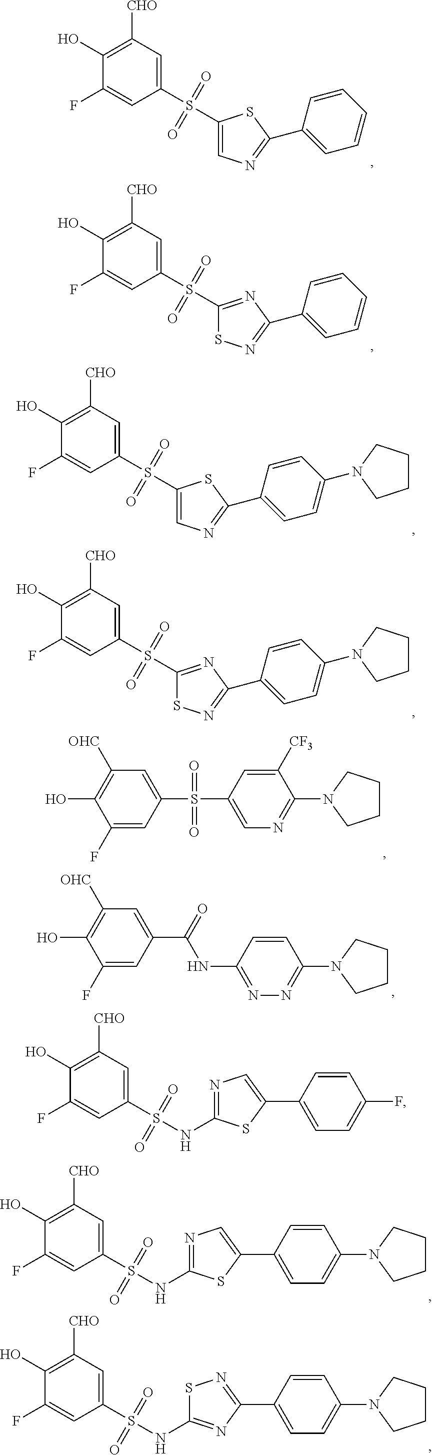

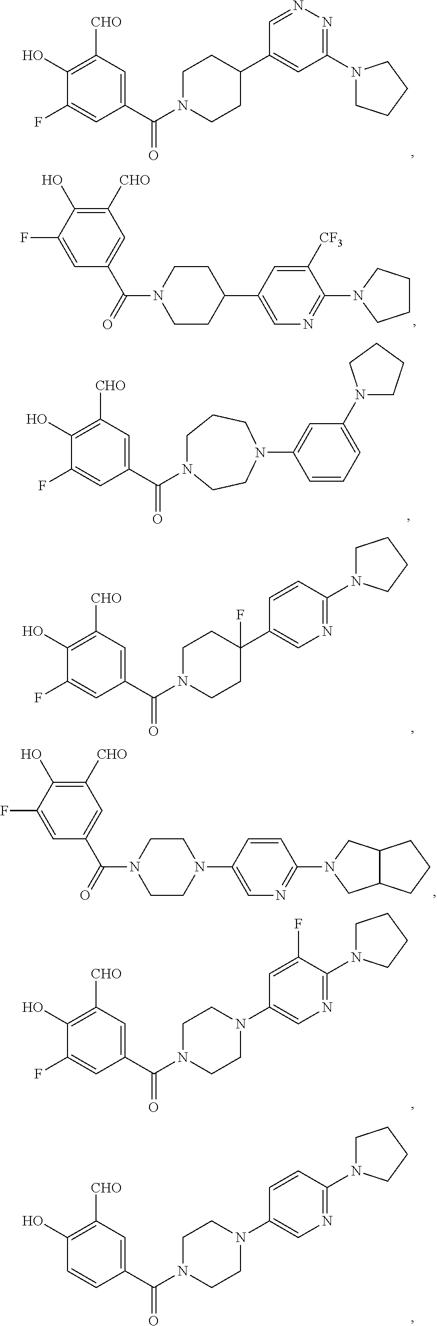

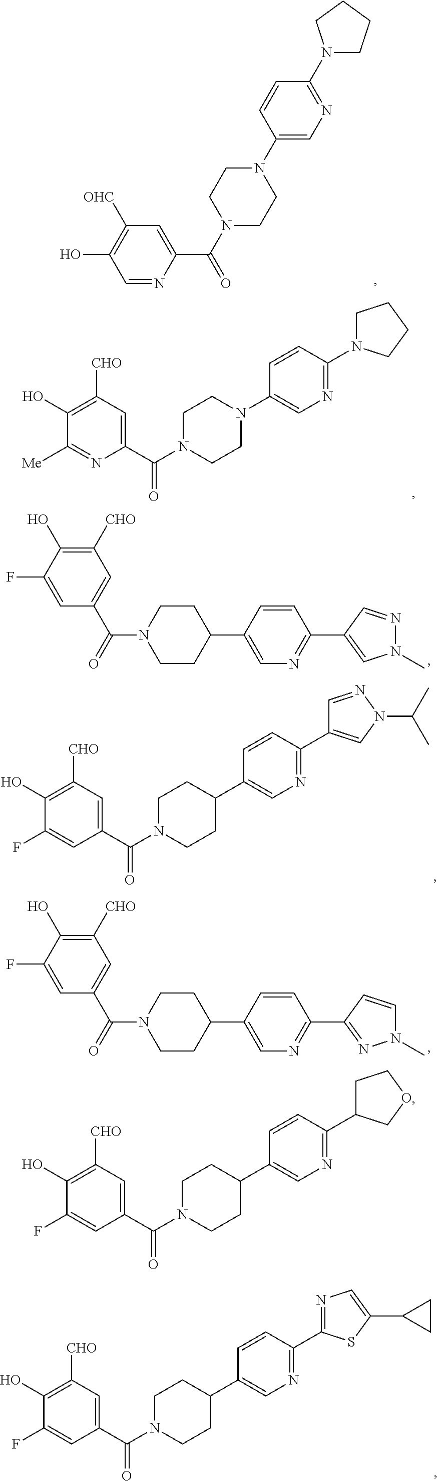

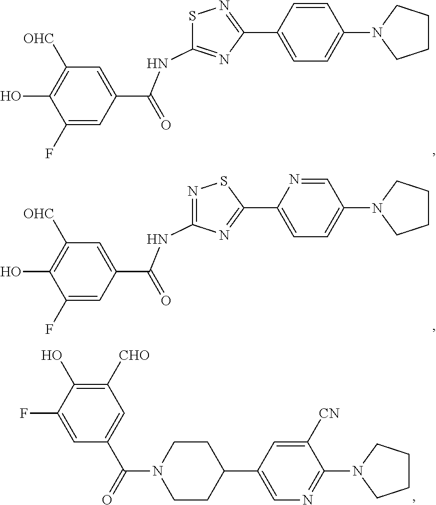

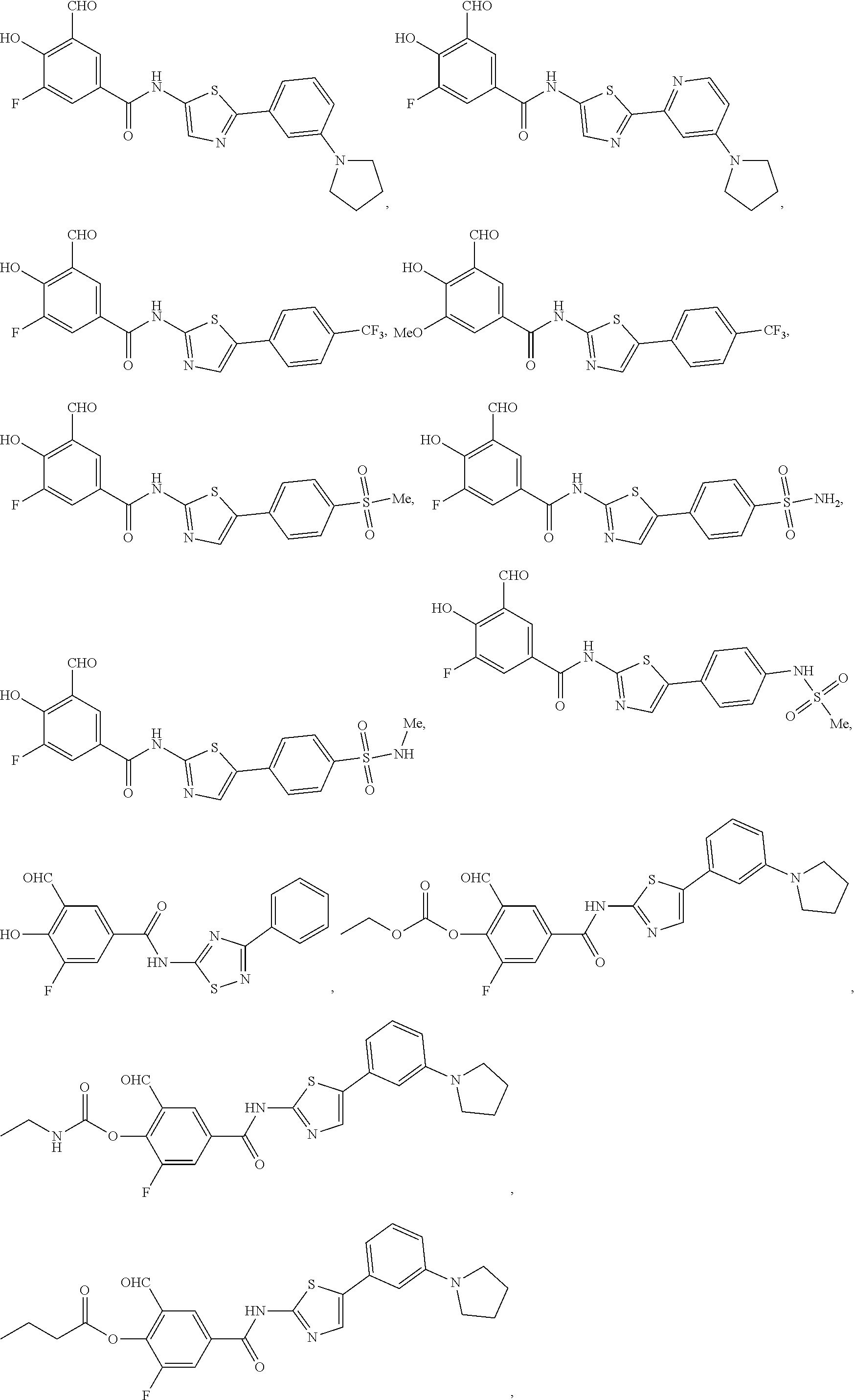

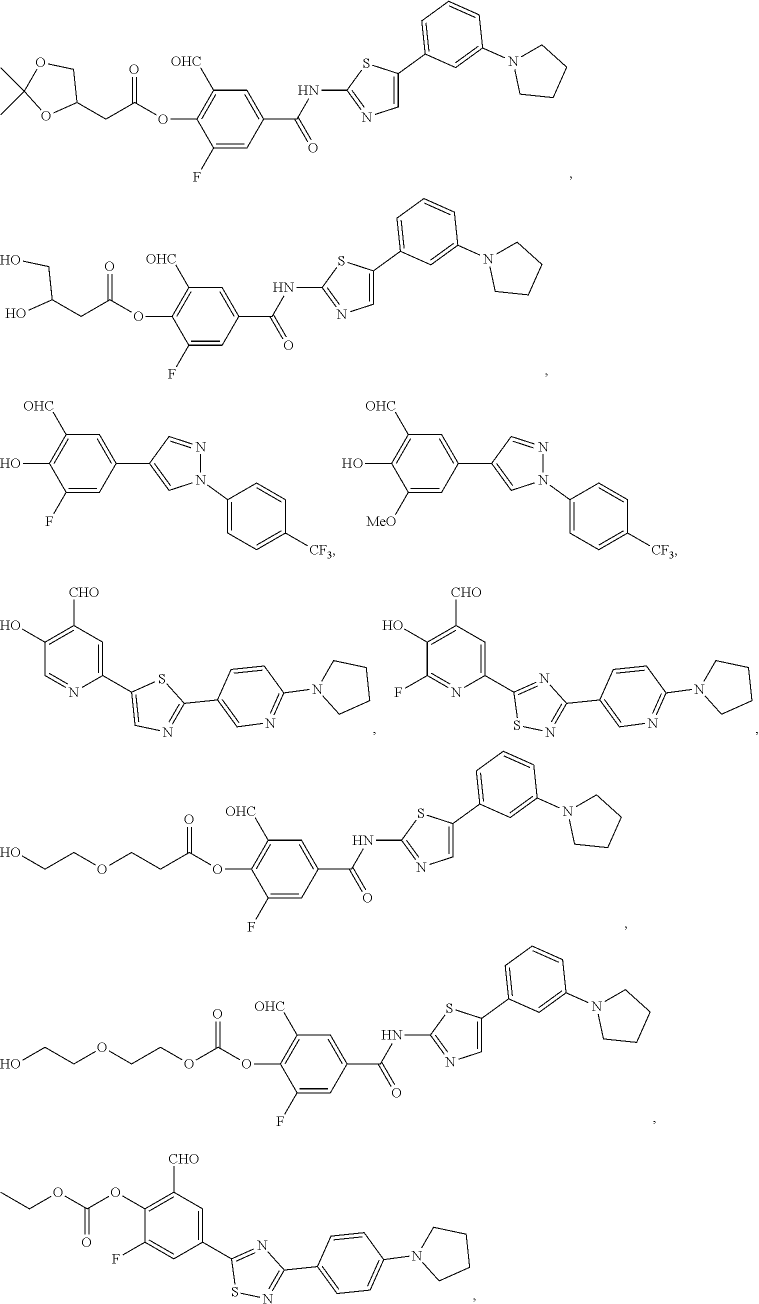

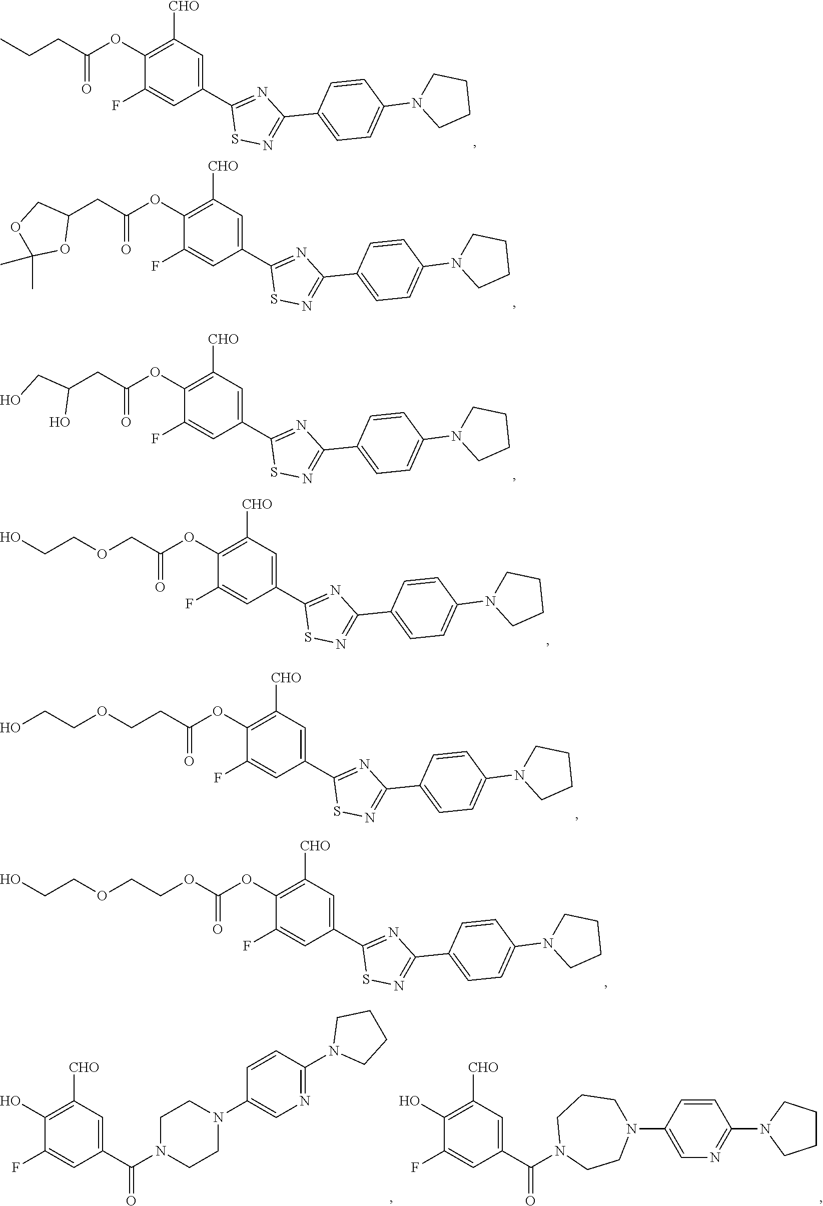

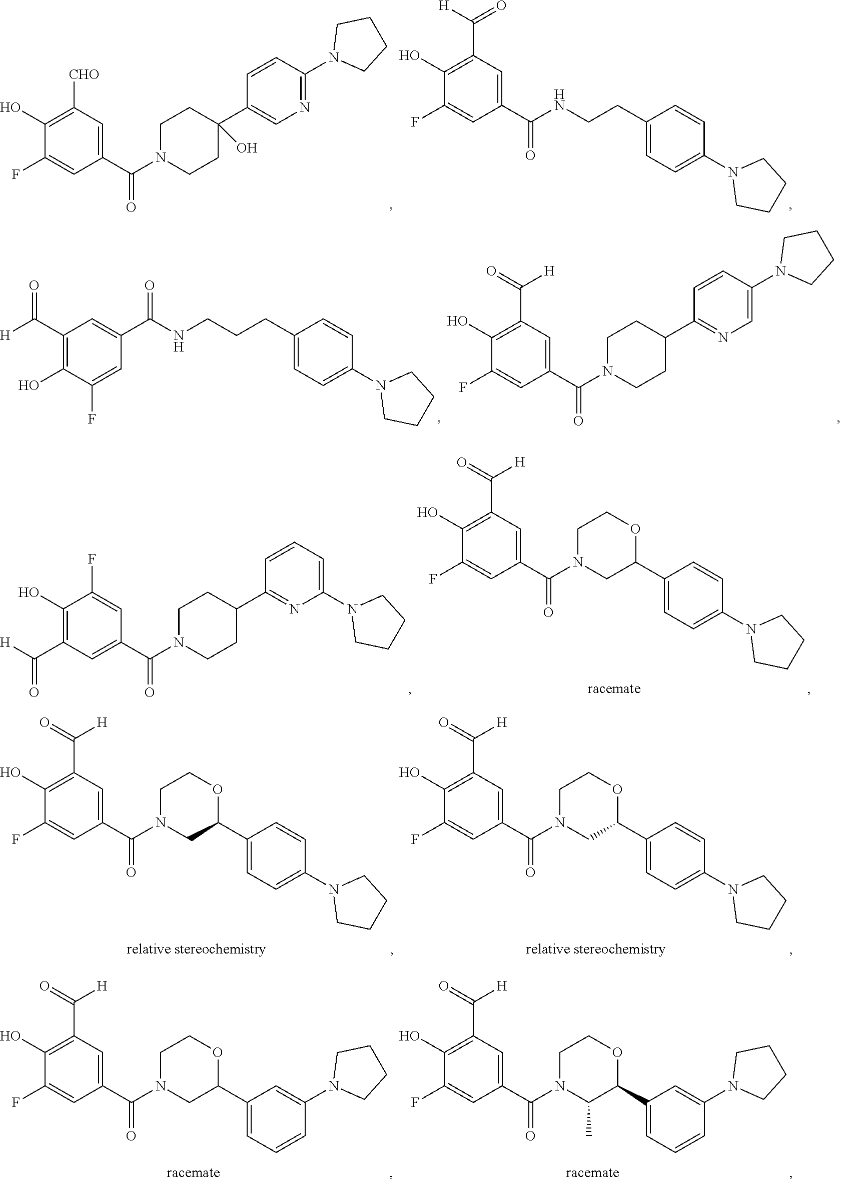

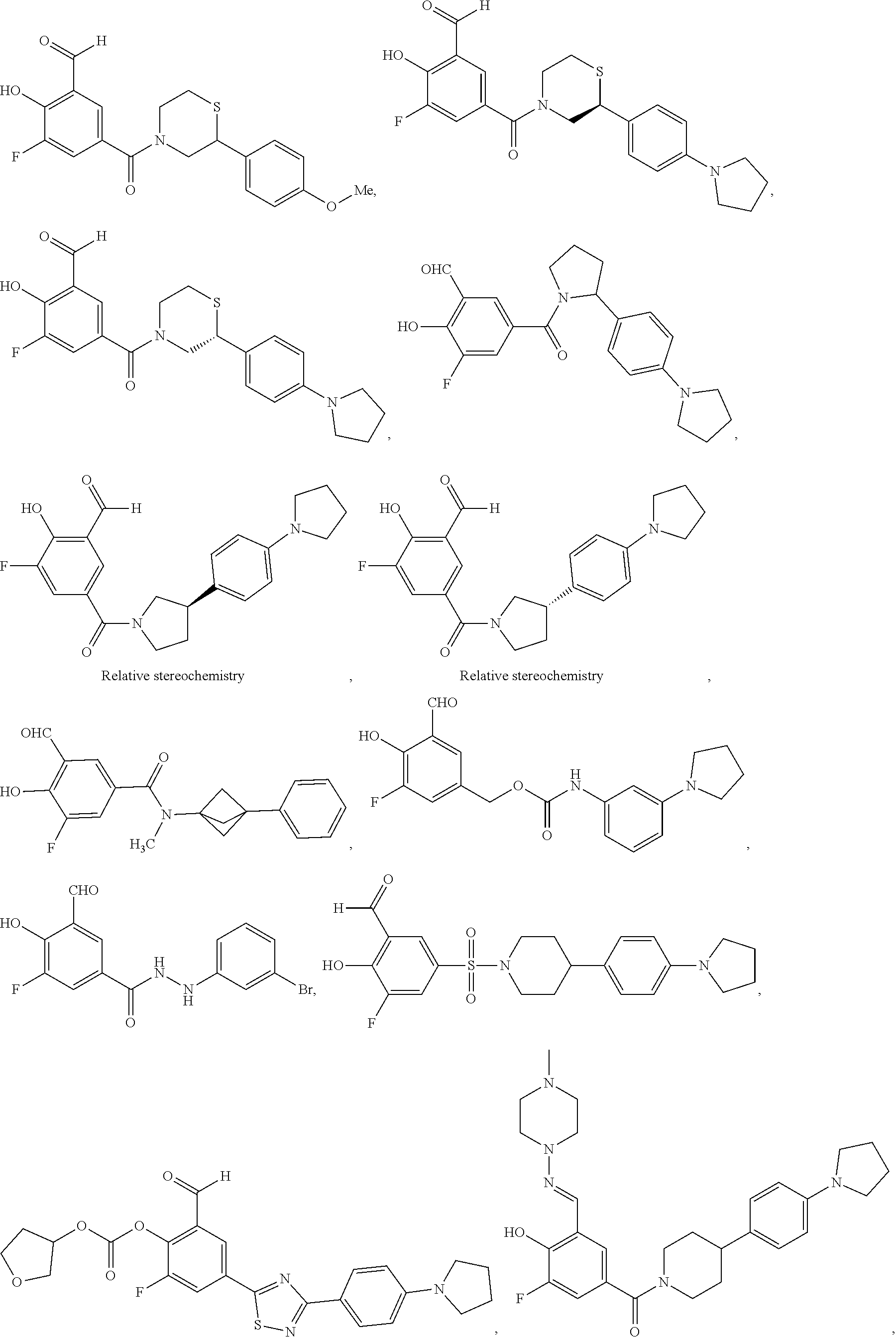

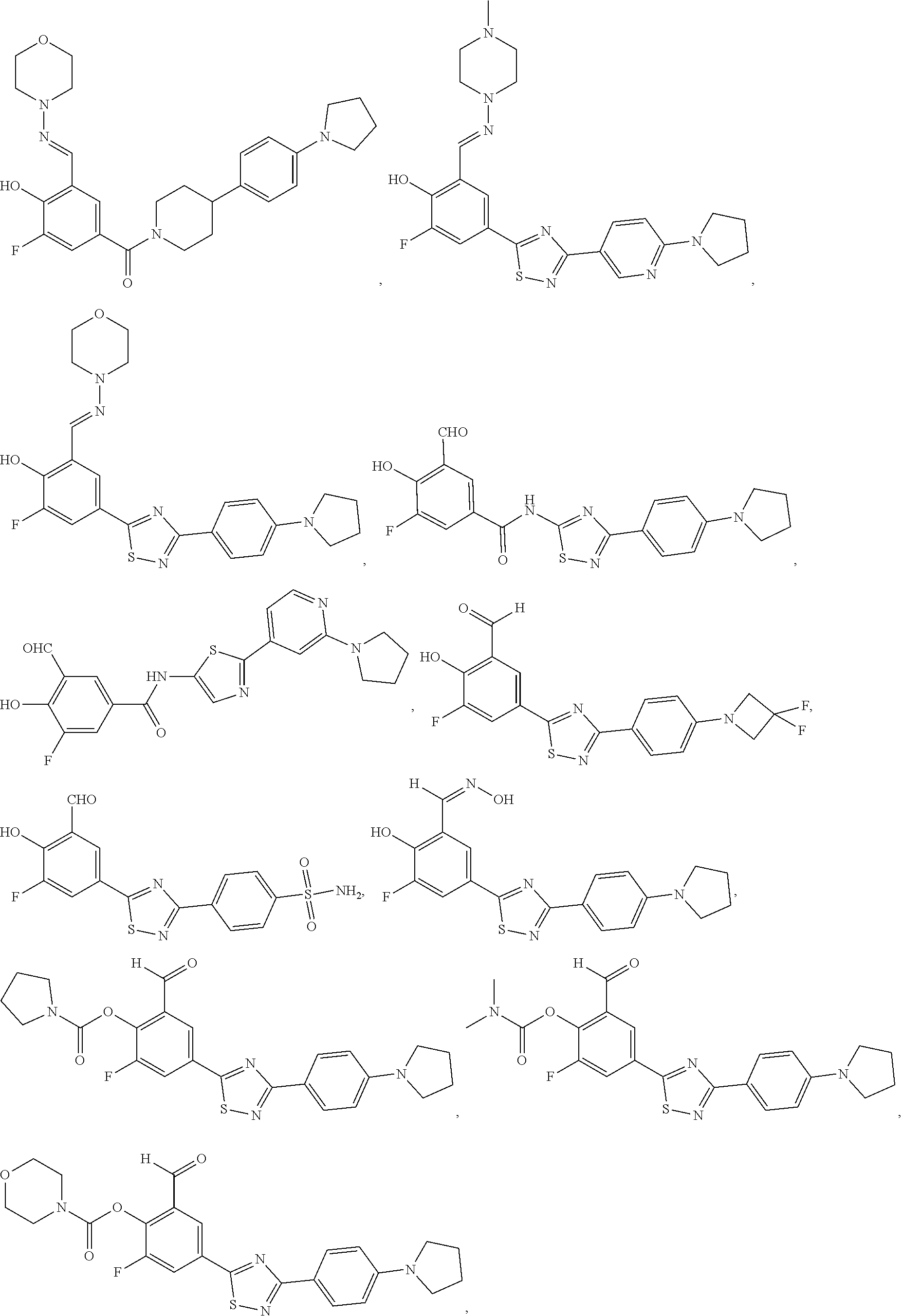

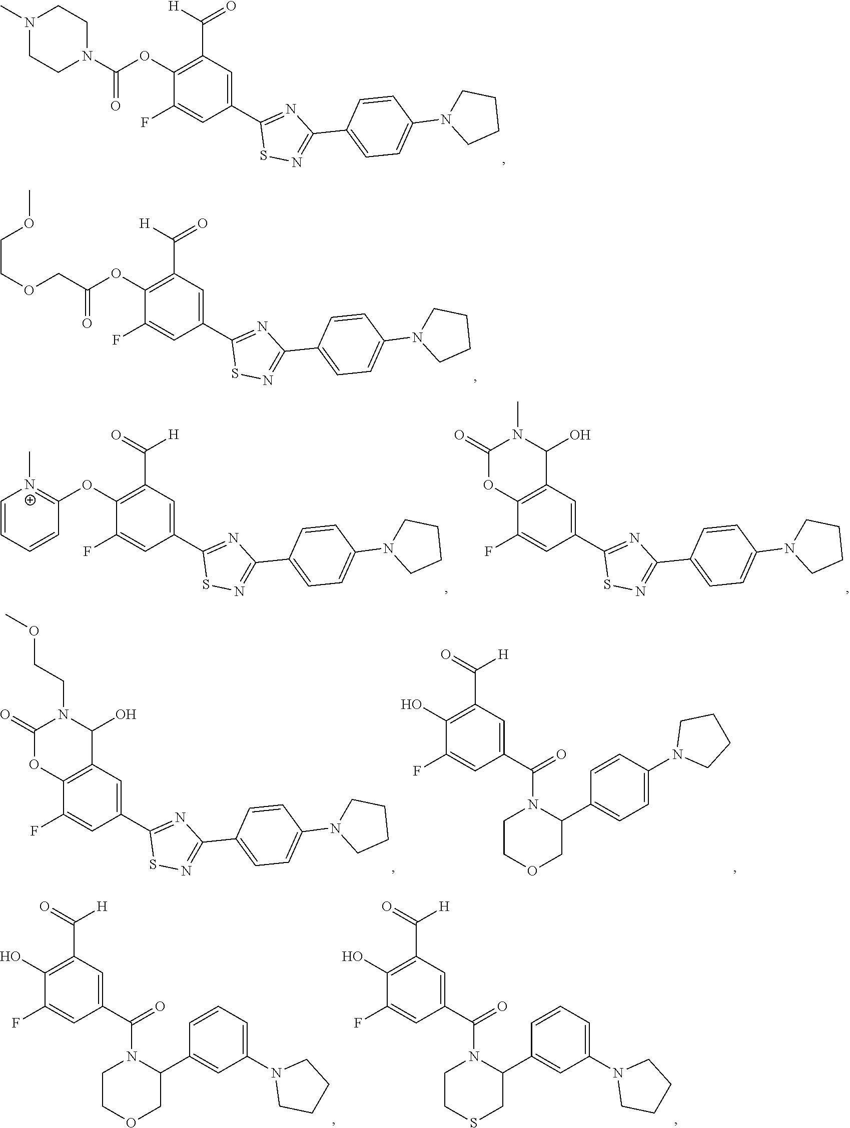

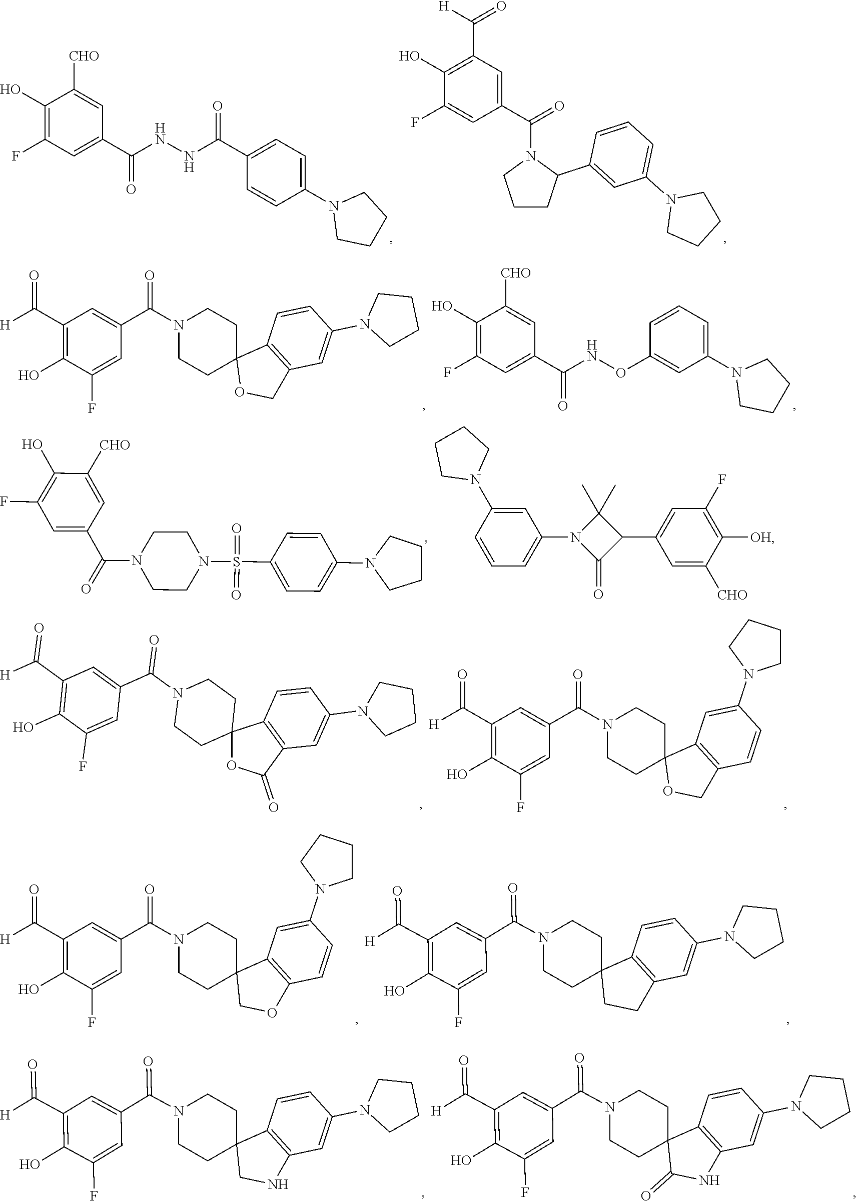

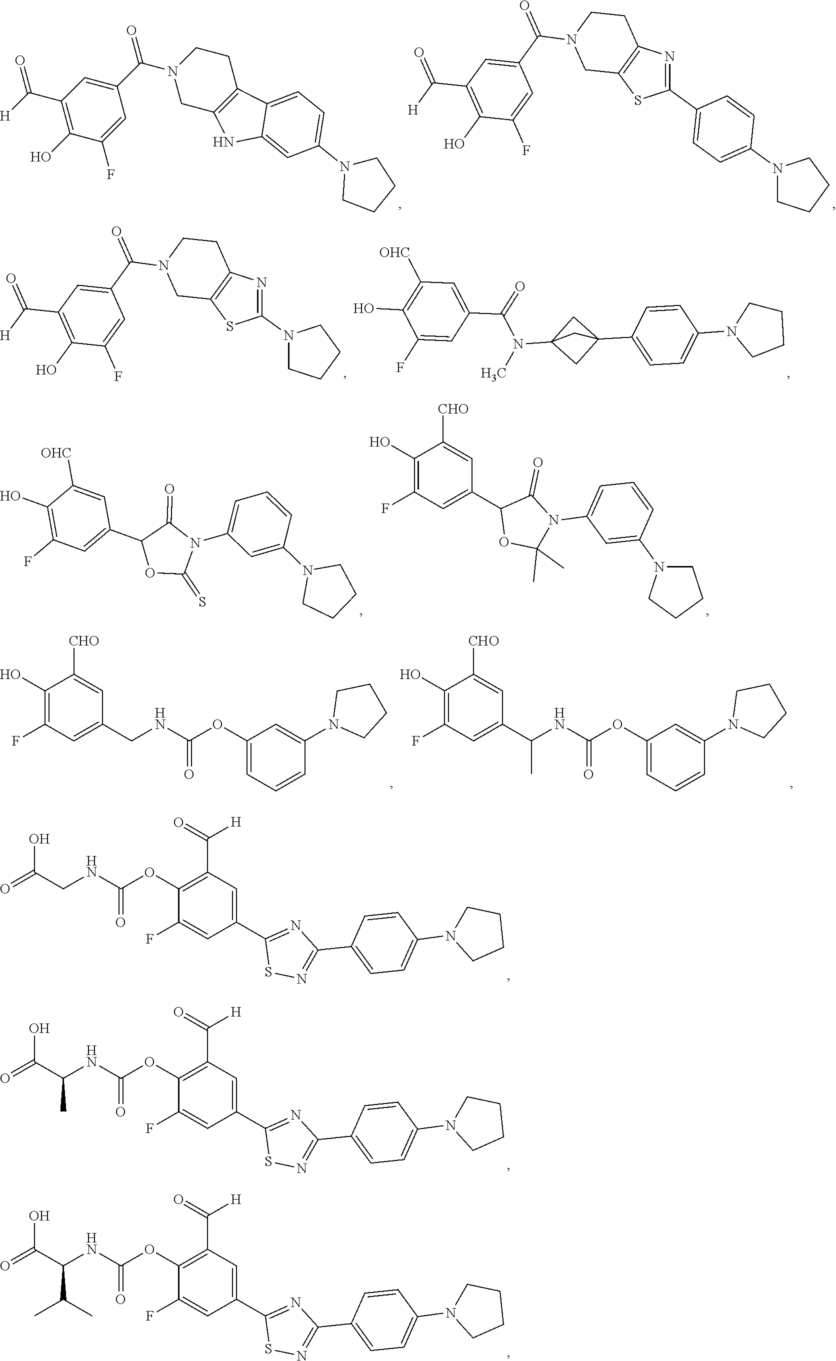

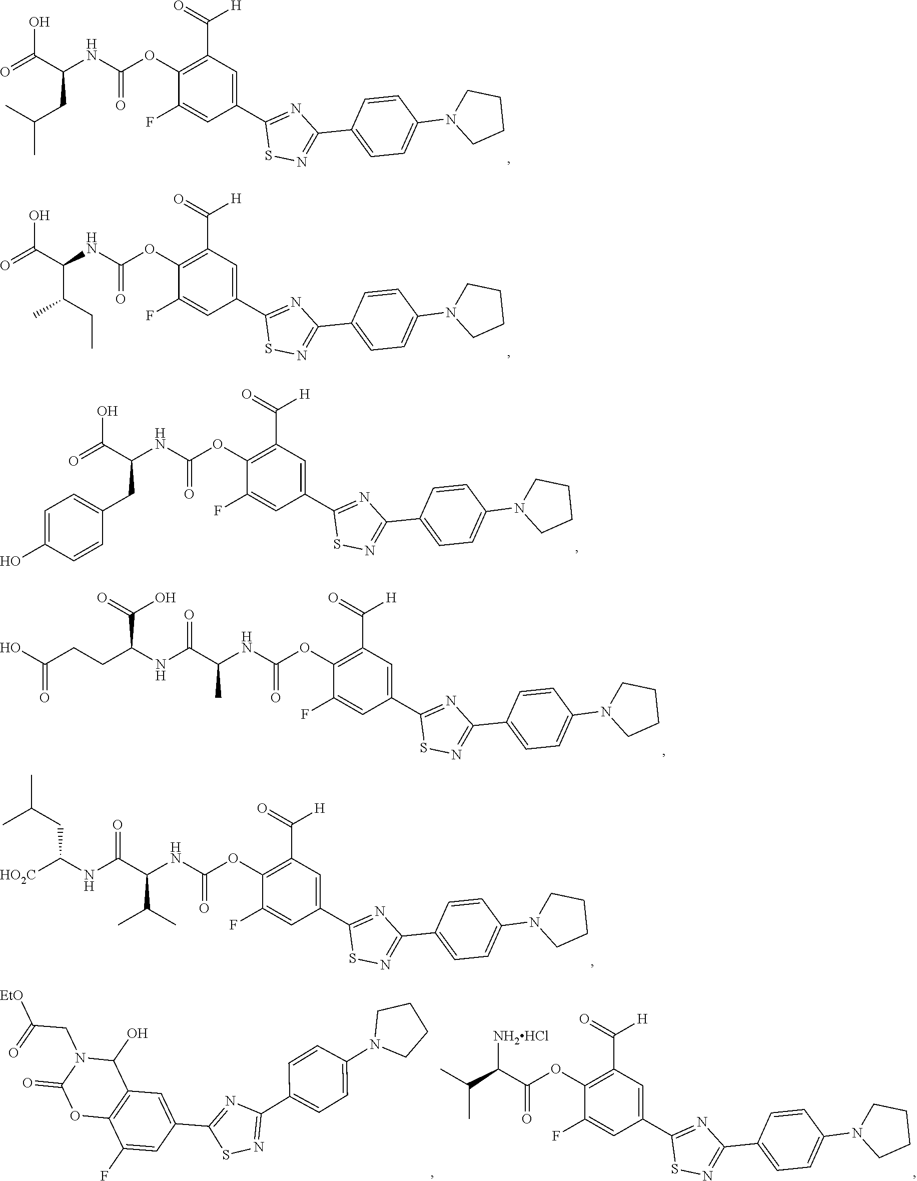

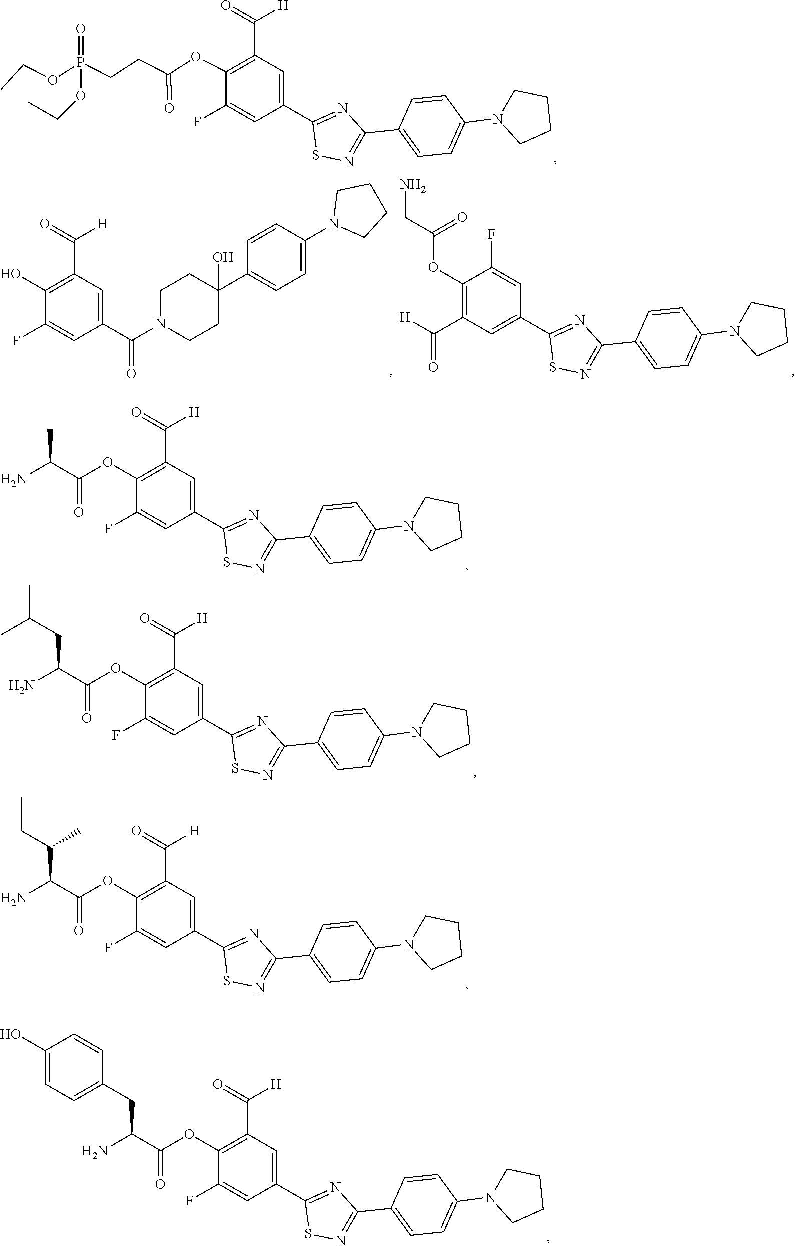

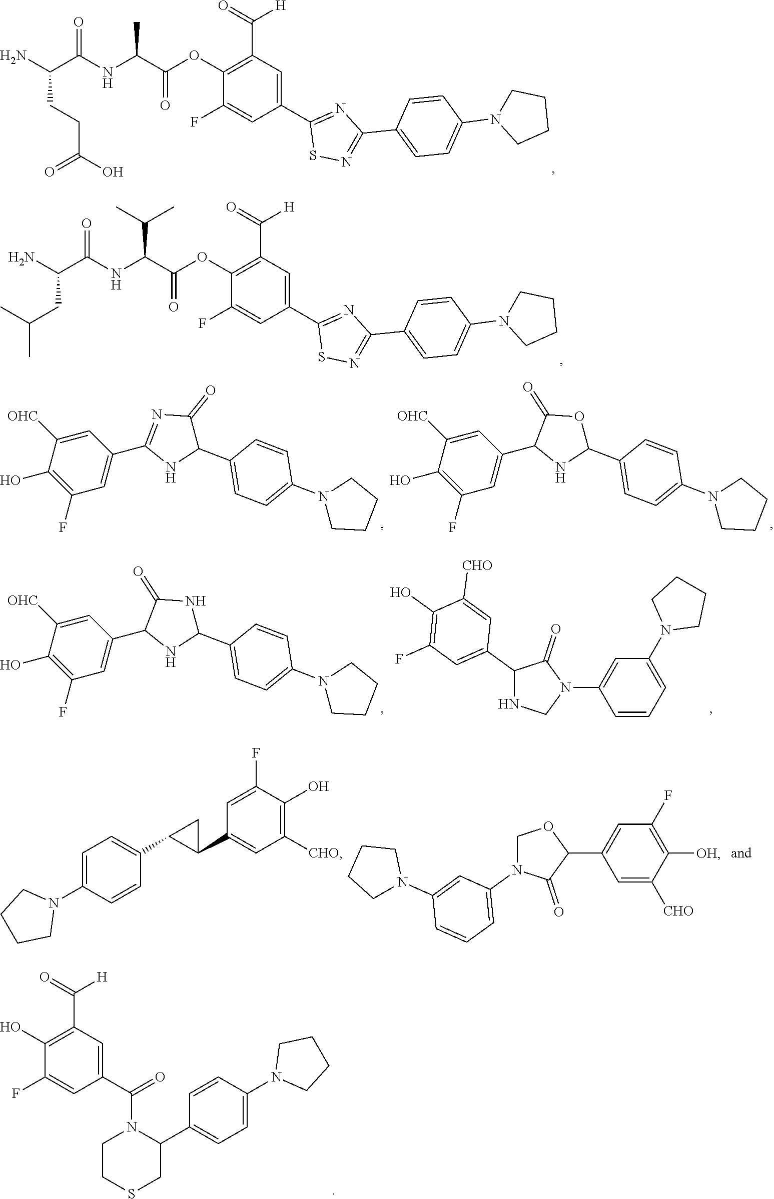

40. A compound of Formula (I-1): 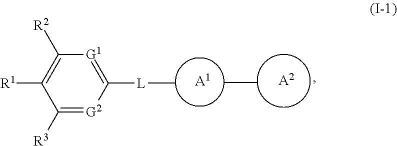
or a tautomer thereof, or a pharmaceutically acceptable salt of any of the foregoing,
wherein
R¹ is R^{1A} and R² is R^{2A}, or R¹ is R^{2A} and R² is R^{1A},
wherein R^{1A} is —OH, —OPO₃H₂, —OCH₂OPO₃H₂, —OC(O)R^{1A1}, —OC(O)OR^{1A1}, —OC(O)NHR^{1A1}, or —OC(O)NR^{1A1}R^{1A2},
wherein R^{1A1} and R^{1A2} are each independently hydrogen, optionally substituted alkyl, optionally substituted alkenyl, optionally substituted alkynyl, optionally substituted cycloalkyl, optionally substituted cycloalkenyl, optionally substituted aryl, optionally substituted heterocyclyl, optionally substituted heteroaryl, or —O₀₋₁(CH₂)ₘO(CH₂)ₙOH,
wherein m and n are each independently 1 or 2, and
R^{2A} is —CHO or —CH═NR^{2A1},
wherein R^{2A1} is optionally substituted heterocyclyl, optionally substituted alkyl, optionally substituted alkenyl, optionally substituted alkynyl, —NR^{2A1A}C(O)R^{2A1B}, —NR^{2A1A}S(O)₂R^{2A1B}, —NR^{2A1A}R^{2A1B}, —OR^{2A1A}, or —NR^{2A1A}C(NR^{2A1B})NR^{2A1C}R^{2A1D}, and
wherein R^{2A1A}, R^{2A1B}, R^{2A1C}, and R^{2A1D} are each independently hydrogen, optionally substituted alkyl, optionally substituted cycloalkyl, or optionally substituted heteroaryl;
R³ is halo, hydrogen, optionally substituted alkyl, or optionally substituted alkoxy;
G¹ and G² are each independently CH or N;
L is a bond, —C(O)NH—*, —NHC(O)—*, —C(R^{4A})(R^{4B})NHC(O)—*, —C(O)—, —S(O)₂—, —S(O)₂NH—*, or 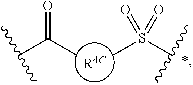
wherein R^{4A} and R^{4B} are each independently hydrogen or optionally substituted alkyl,
R^{4C} is optionally substituted aryl, optionally substituted cycloalkyl, optionally substituted cycloalkenyl, optionally substituted heteroaryl, or optionally substituted heterocyclyl, and
represents the point of attachment to A¹; and
A¹ and A² are each independently optionally substituted aryl, optionally substituted cycloalkyl, optionally substituted cycloalkenyl, optionally substituted heteroaryl, or optionally substituted heterocyclyl;
provided that
when R¹ is —OH, R³ is fluoro, L is a bond, and A¹ is optionally substituted 5-membered heteroaryl, then A² is not optionally substituted phenyl, optionally substituted pyridinyl, optionally substituted pyrazinyl, or 2,3-dihydrobenzo[b][1,4]dioxin-6-yl;
when L is —C(O)NH—*, then A¹ is not optionally substituted phenyl, optionally substituted pyridinyl, or pyrimidinyl;
when R³ is hydrogen, methyl, isobutyl, or methoxy, then L is not a bond; and
the compound of Formula (I) is not 3-fluoro-5-formyl-4-hydroxy-N-(4-(pyrrolidin-1-yl)phenyl)benzenesulfonamide, 5-(4-(5-fluoropyridin-2-yl)piperazine-1-carbonyl)-2-hydroxy-3-methylbenzaldehyde, 5-(3-(1λ⁴,2λ²,4-triazol-1-yl)azetidine-1-carbonyl)-2-hydroxy-3-methylbenzaldehyde, tert-butyl (3-(1-(3-formyl-4-hydroxybenzoyl)piperidin-4-yl)benzyl)carbamate, or 5-(4-cyclopropyl-3-oxopiperazine-1-carbonyl)-2-hydroxy-3-methylbenzaldehyde, or a salt of any of the foregoing.

41. The compound of claim 40, or a tautomer thereof, or a pharmaceutically acceptable salt of any of the foregoing, wherein the compound is selected from the group consisting of

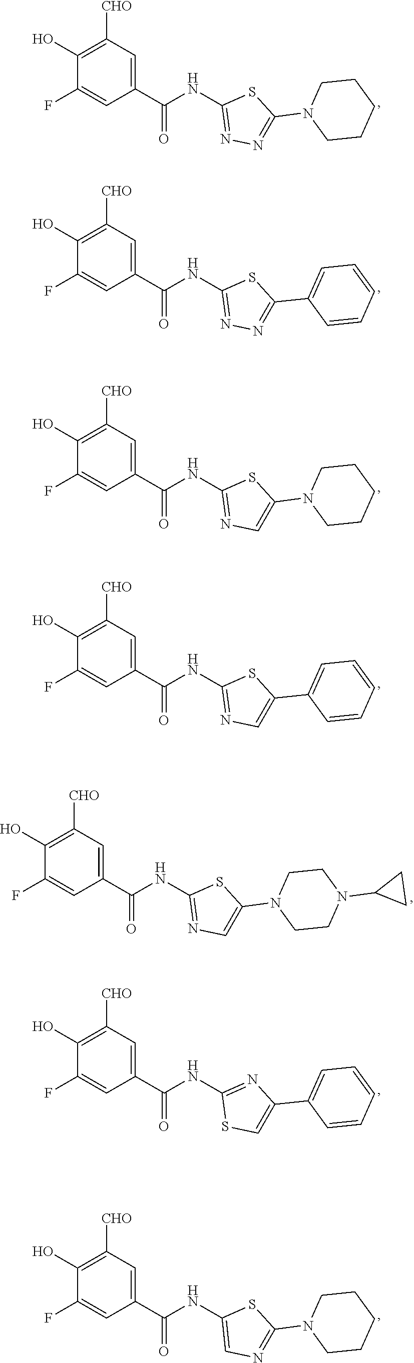

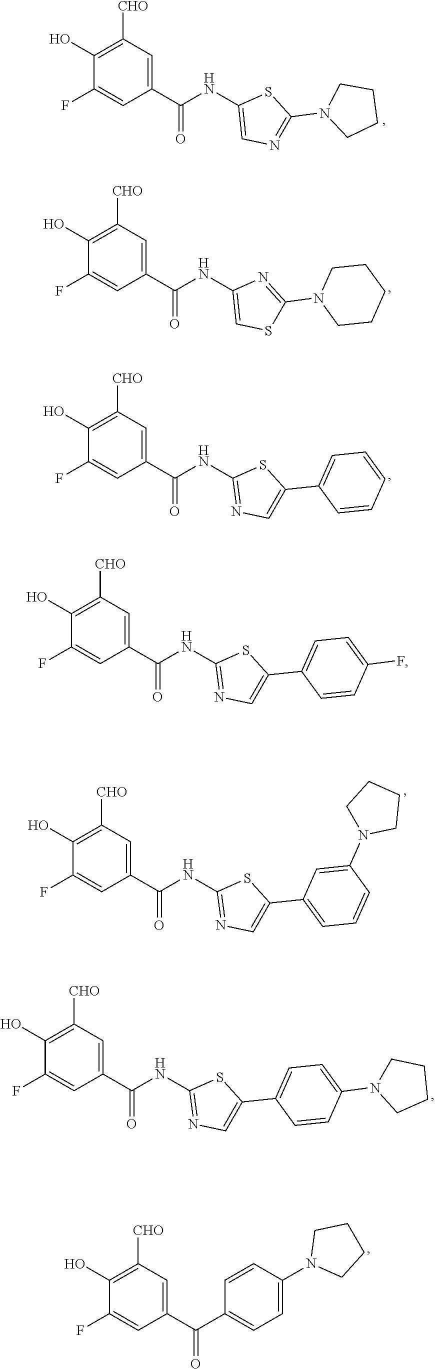

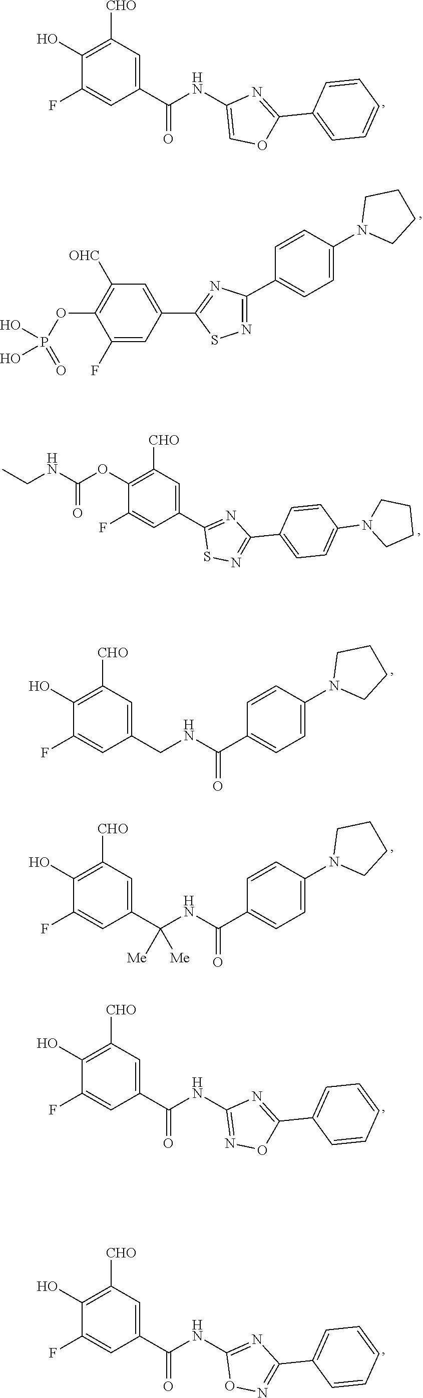

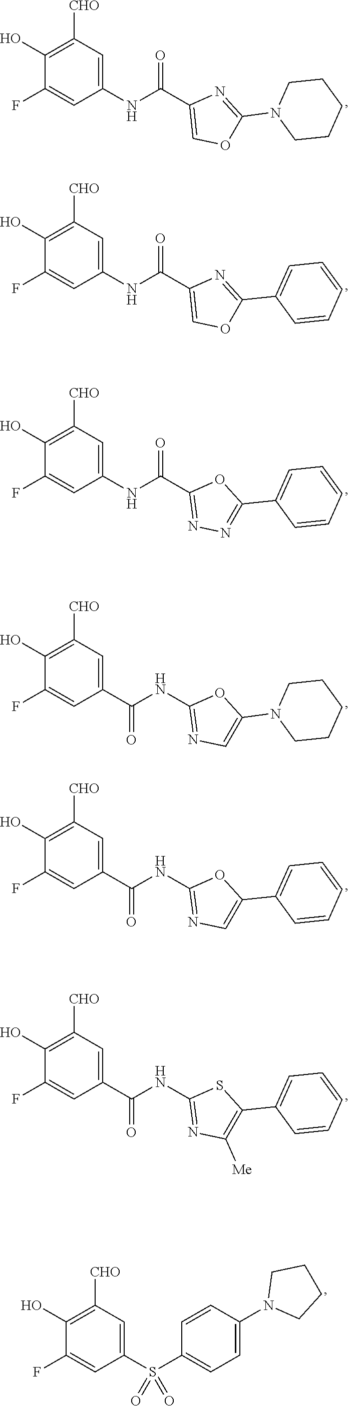

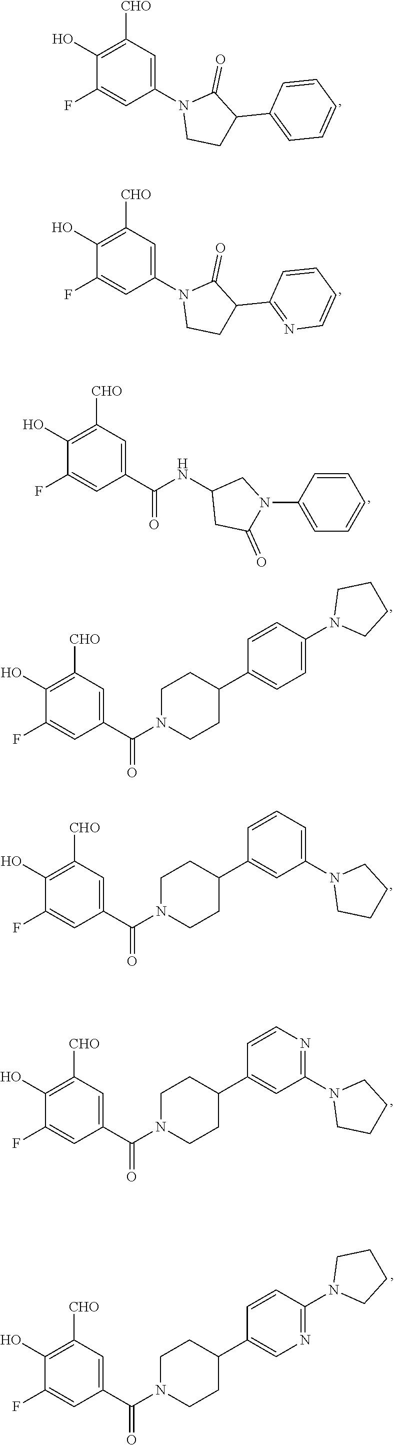

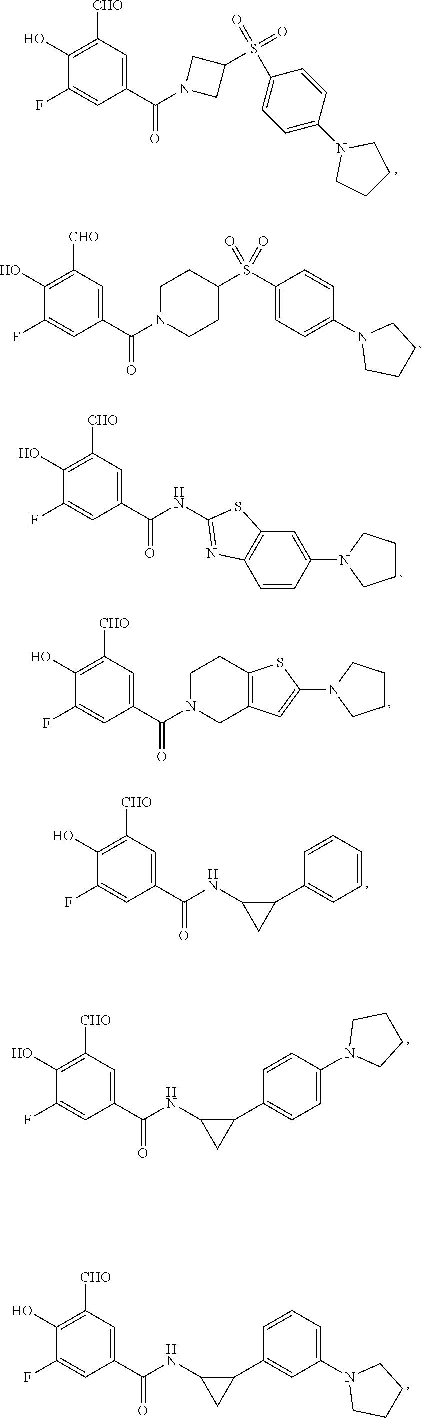

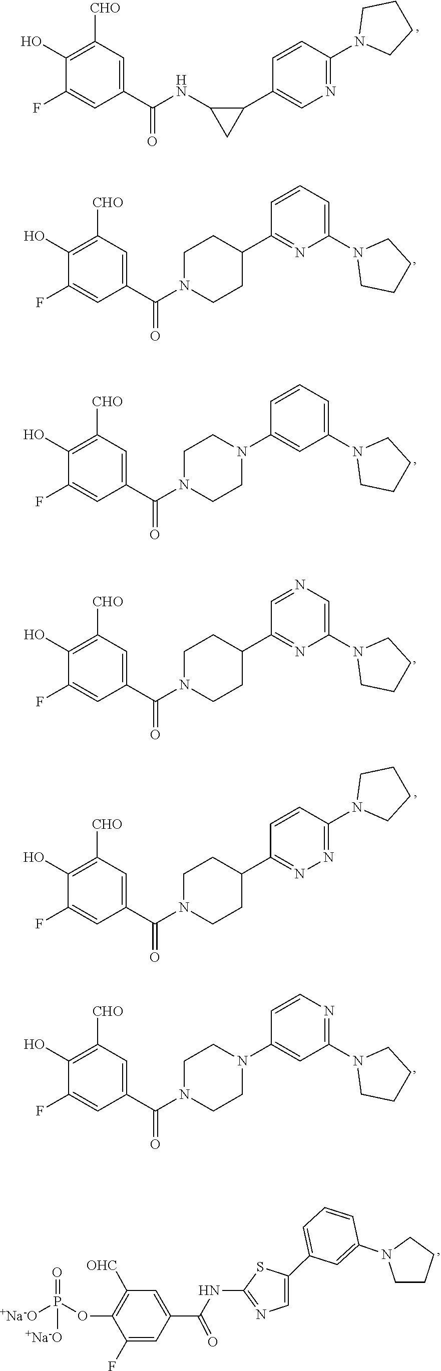

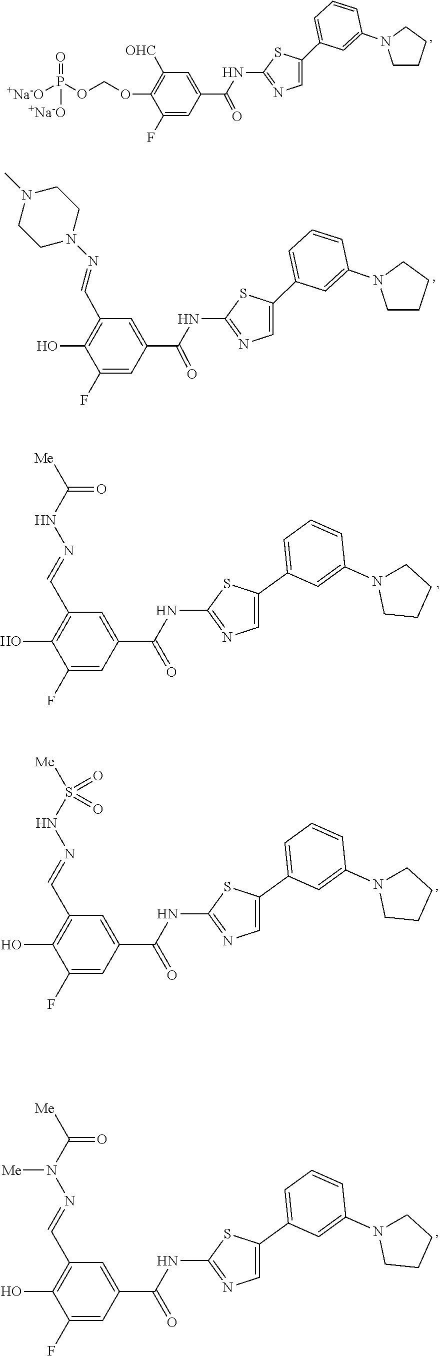

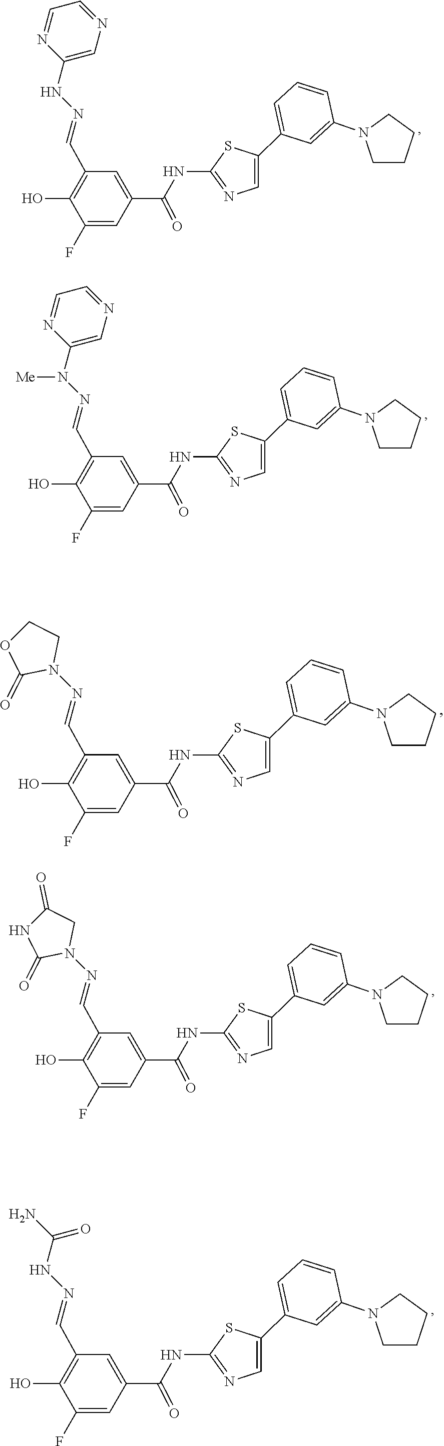

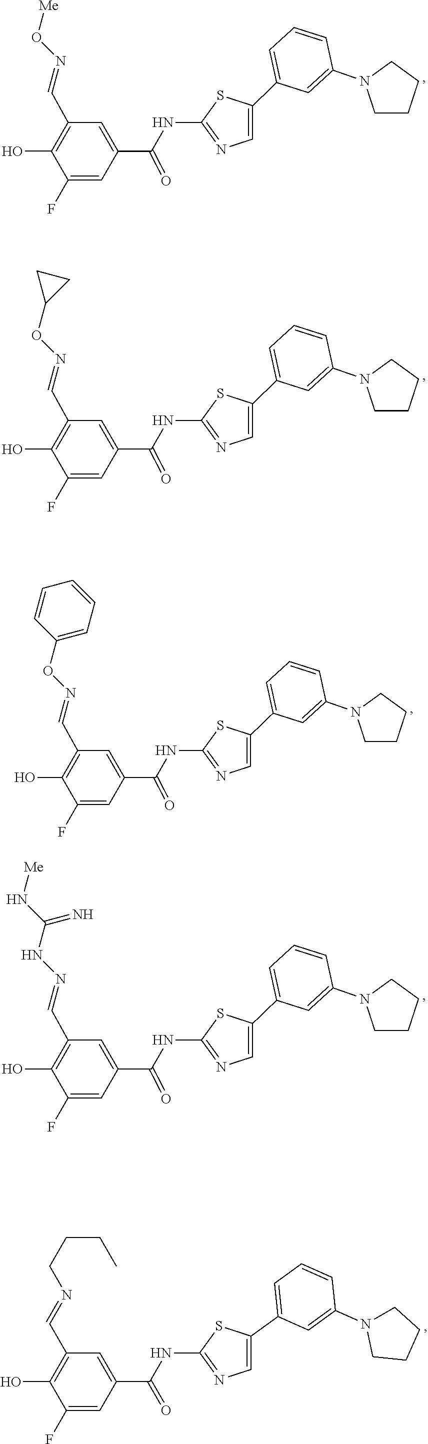 [SMILES: CO/N=C/c1cc(C(=O)Nc2ncc(-c3cccc(N4CCCC4)c3)s2)cc(F)c1O ; O=C(Nc1ncc(-c2cccc(N3CCCC3)c2)s1)c1cc(F)c(O)c(/C=N/OC2CC2)c1 ; O=C(Nc1ncc(-c2cccc(N3CCCC3)c2)s1)c1cc(F)c(O)c(/C=N/Oc2ccccc2)c1 ; CNC(=N)N/N=C/c1cc(C(=O)Nc2ncc(-c3cccc(N4CCCC4)c3)s2)cc(F)c1O ; CCCC/N=C/c1cc(C(=O)Nc2ncc(-c3cccc(N4CCCC4)c3)s2)cc(F)c1O]

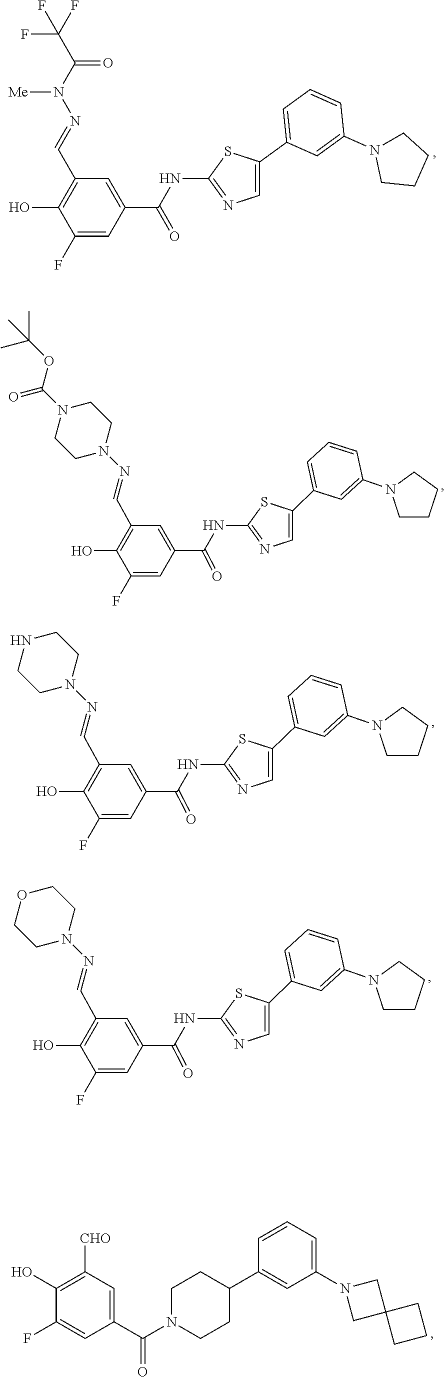 [SMILES: CN(/N=C/c1cc(C(=O)Nc2ncc(-c3cccc(N4CCCC4)c3)s2)cc(F)c1O)C(=O)C(F)(F)F ; CC(C)(C)OC(=O)N1CCN(/N=C/c2cc(C(=O)Nc3ncc(-c4cccc(N5CCCC5)c4)s3)cc(F)c2O)CC1 ; O=C(Nc1ncc(-c2cccc(N3CCCC3)c2)s1)c1cc(F)c(O)c(/C=N/N2CCNCC2)c1 ; O=C(Nc1ncc(-c2cccc(N3CCCC3)c2)s1)c1cc(F)c(O)c(/C=N/N2CCOCC2)c1 ; O=Cc1cc(C(=O)N2CCC(c3cccc(N4CC5(CCC5)C4)c3)CC2)cc(F)c1O]

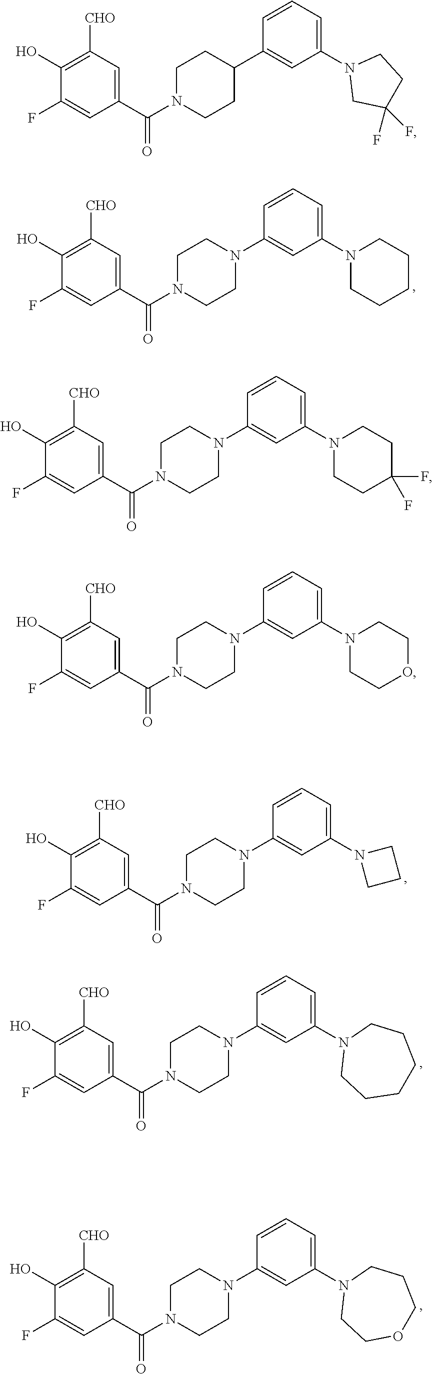

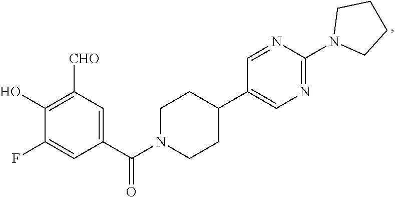 [SMILES: O=Cc1cc(C(=O)N2CCC(c3cnc(N4CCCC4)nc3)CC2)cc(F)c1O]

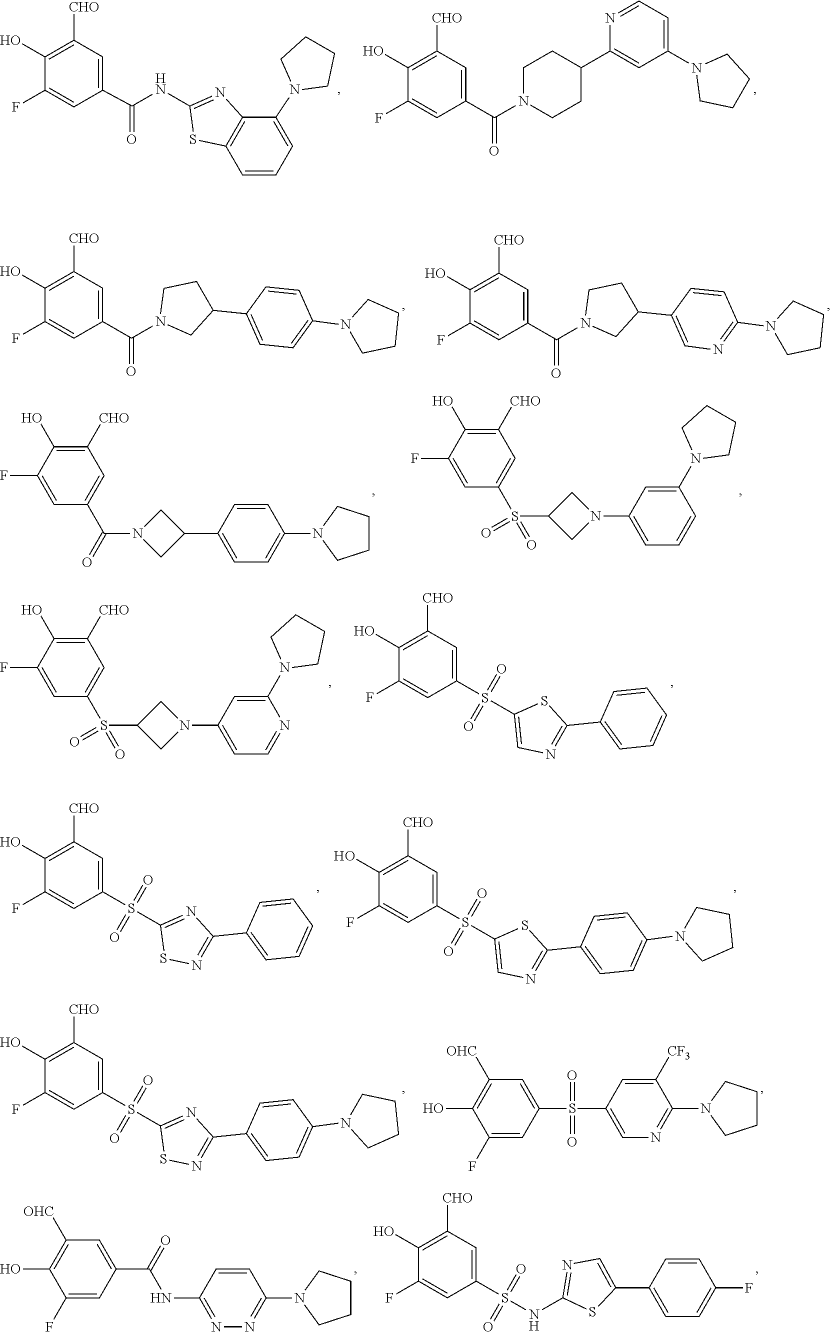

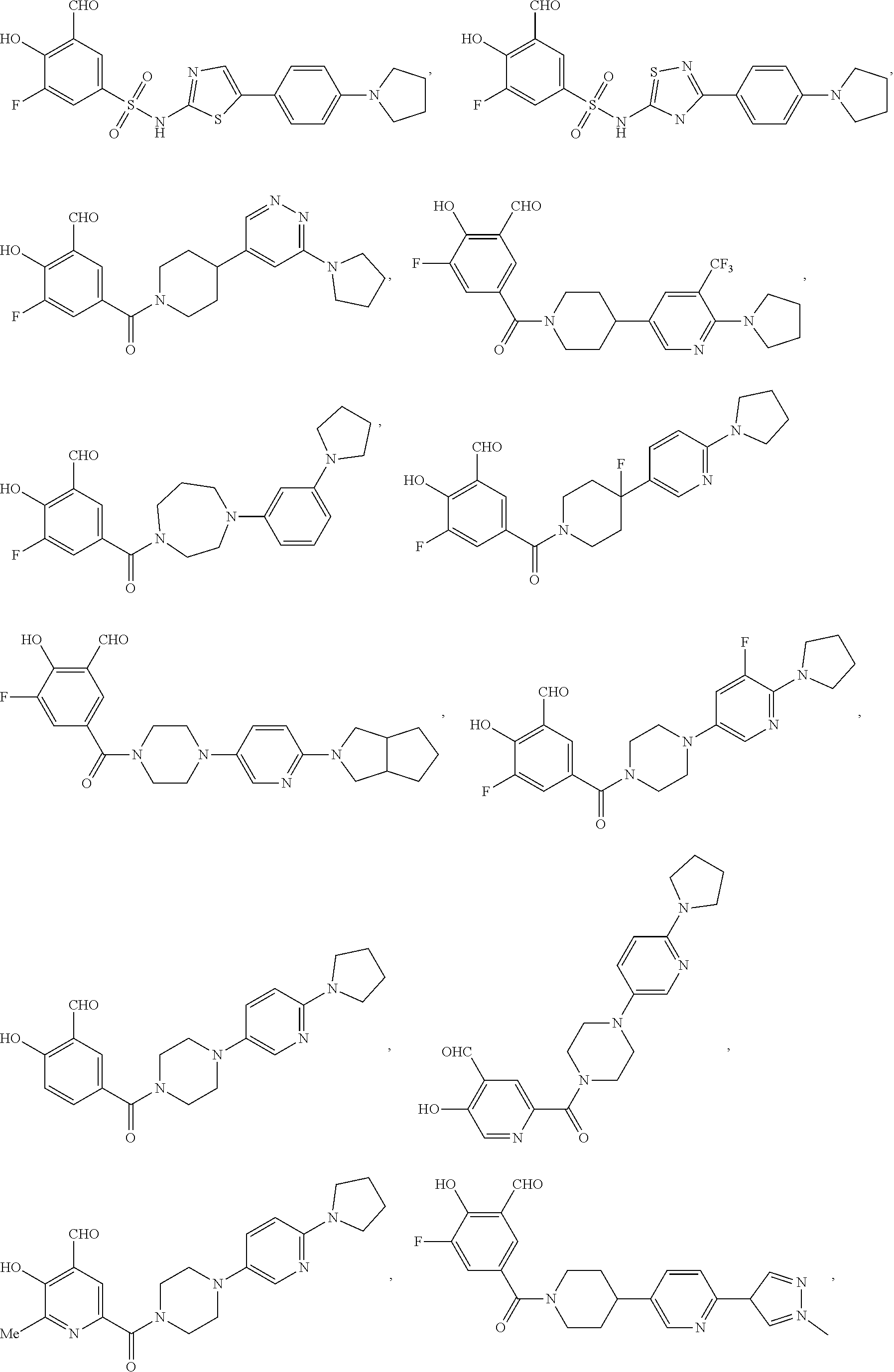

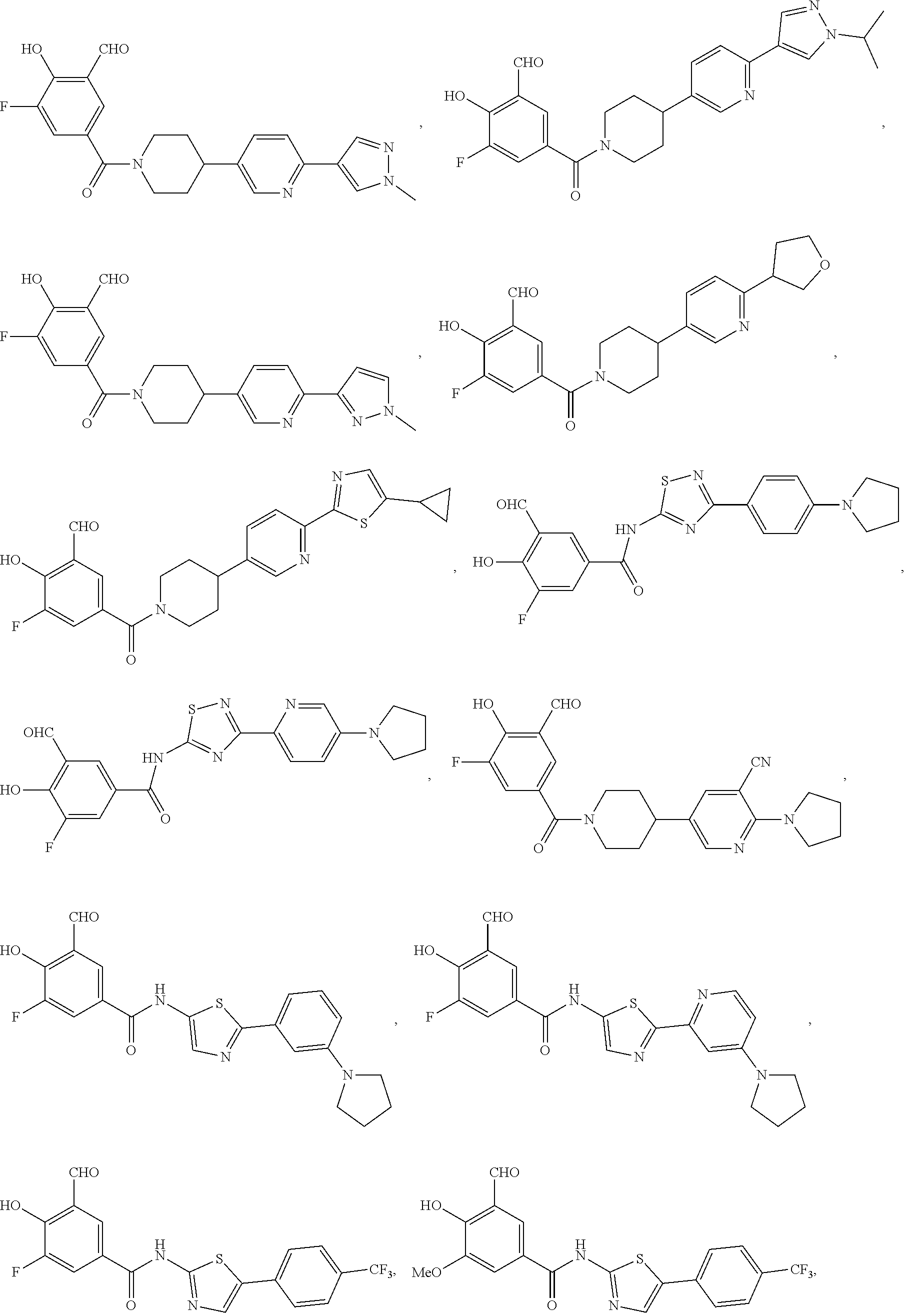

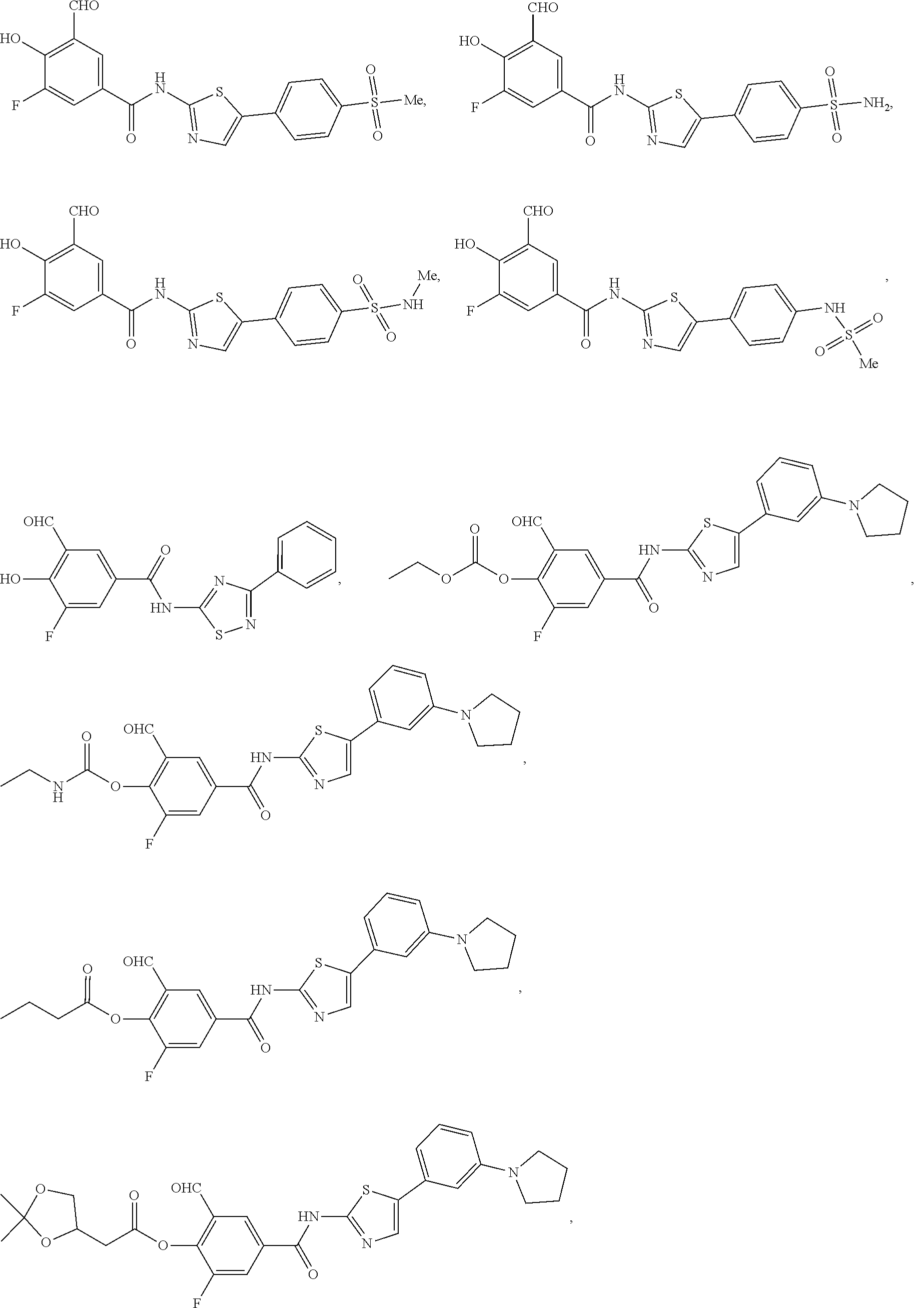

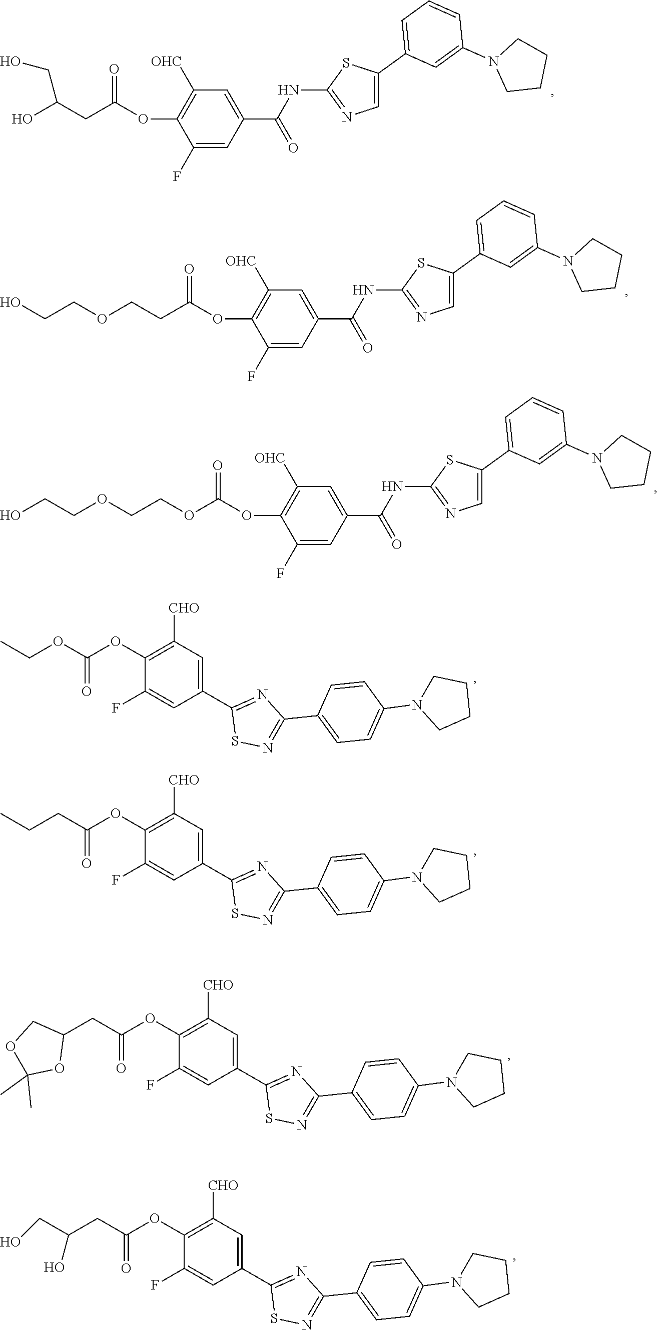

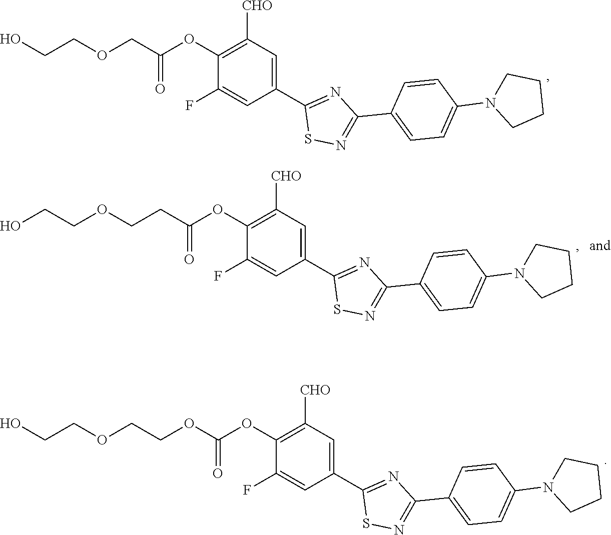 [SMILES: O=Cc1cc(-c2nc(-c3ccc(N4CCCC4)cc3)ns2)cc(F)c1OC(=O)COCCO ; O=Cc1cc(-c2nc(-c3ccc(N4CCCC4)cc3)ns2)cc(F)c1OC(=O)CCOCCO ; O=Cc1cc(-c2nc(-c3ccc(N4CCCC4)cc3)ns2)cc(F)c1OC(=O)OCCOCCO]

42. A pharmaceutical composition comprising at least one compound according to claim 1, or a tautomer thereof, or a pharmaceutically acceptable salt of any of the foregoing, optionally further comprising a pharmaceutically acceptable excipient.

43. A method of treating a disease or condition associated with TLR2 or TLR2 heterodimerization, comprising administering to a subject in need of such treatment an effective amount of at least one compound according to claim 1, or a tautomer thereof, or a pharmaceutically acceptable salt of any of the foregoing, or a pharmaceutical composition of claim 42.

44. The method of claim 43, wherein the disease or condition is selected from the group consisting of: Alzheimer's disease, Parkinson's disease, fronto-temporal dementia, dementia with Lewy bodies (Lewy body disease), Parkinson's disease with dementia, multiple system atrophy, amyotrophic lateral sclerosis, Huntington's disease, Progressive Supranuclear Palsy (PSP), Niemann-Pick disease type C, Guillain-Barré syndrome (GBS), Barrett's esophagus, inflammatory diseases, asthma, chronic obstructive pulmonary disease (COPD), chronic peptic ulcers, irritable bowel disease, tuberculosis, rheumatoid arthritis, osteoarthritis, chronic sinusitis, hepatitis, hepatitis B, hepatitis C, gout, lupus, pleurisy, eczema, gastritis, psoriasis, psoriatic arthritis, vasculitis, laryngitis, allergic reactions, multiple sclerosis, Crohn's disease, traumatic brain injury, CIDP (chronic inflammatory demyelinating polyneuropathy), stroke, ischemic heart disease, atopic dermatitis, acne vulgaris, rosacea, non-alcoholic fatty liver disease, non-alcoholic steatohepatisis, corneal wounds, corneal disorders, corneal HSV, Stargardt disease (Juvenile macular degeneration), age-related macular degeneration, sepsis, diabetic wounds, herpes simplex virus, and anti-fungal, anti-bacterial, anitviral and antitumor diseases or conditions.

45-48. (canceled)

49. A method of interfering with the heterodimerization of TLR2 in a cell, or modulating, preventing, slowing, reversing, or inhibiting TLR2 heterodimerization in a cell, comprising contacting the cell with an effective amount of at least one compound according to claim 1, or a tautomer thereof, or a pharmaceutically acceptable salt of any of the foregoing, and/or with at least one pharmaceutical composition according to claim 42, wherein the contacting is in vitro, ex vivo, or in vivo.

50. A method of inhibiting TLR2 activation in a cell, comprising contacting the cell with an effective amount of at least one compound according to claim 1, or a tautomer thereof, or a pharmaceutically acceptable salt of any of the foregoing, and/or with at least one pharmaceutical composition according to claim 42, wherein the contacting is in vitro, ex vivo, or in vivo.

51. A method of treating a disease or condition associated with inhibition of TLR9, comprising administering to a subject in need of such treatment an effective amount of at least one compound according to claim 1, or a tautomer thereof, or a pharmaceutically acceptable salt of any of the foregoing, or a pharmaceutical composition of claim 42.

52. The method of claim 43, wherein the disease or condition is central nervous system (CNS) or peripheral disorder.

53. The method of claim 52, wherein the disease or condition is Parkinson's disease, Amyotrophic lateral sclerosis, Guillain-Barre syndrome, spinal cord injury, multiple sclerosis, multiple forms of tissue injury, chronic pain, or psoriasis.

54-59. (canceled)

60. A method of inhibiting TLR9 in a cell, comprising contacting the cell with an effective amount of at least one compound according to claim 1, or a tautomer thereof, or a pharmaceutically acceptable salt of any of the foregoing, and/or with at least one pharmaceutical composition according to claim 42, wherein the contacting is in vitro, ex vivo, or in vivo.
